(19) United States
(12) Patent Application Publication
(10) Pub. No.: US 2026/0182242 A1
(43) Pub. Date: Jun. 25, 2026

(54) PLURALITY OF HOST MATERIALS, AN ORGANIC ELECTROLUMINESCENT COMPOUND, AND ORGANIC ELECTROLUMINESCENT DEVICE COMPRISING THE SAME

(71) Applicant: DuPont Specialty Materials Korea Ltd., Chungcheongnam-do (KR)

(72) Inventors: HaeYeon KIM, Gyeonggi-do (KR); Yoo-Jin DOH, Gyeonggi-do (KR); Chi-Sik KIM, Gyeonggi-do (KR); Mi-Ja LEE, Gyeonggi-do (KR); Young-Kwang KIM, Gyeonggi-do (KR); YoSeop KIM, Gyeonggi-do (KR)

(21) Appl. No.: 19/392,241

(22) Filed: Nov. 18, 2025

(30) Foreign Application Priority Data
Dec. 20, 2024 (KR) 10-2024-0192151
Jun. 10, 2025 (KR) 10-2025-0075344
Oct. 20, 2025 (KR) 10-2025-0151410

(51) Int. Cl. H10K 85/60; C07C 15/30; C07C 15/38; C09K 11/02; H10K 50/11; H10K 101/00

(52) U.S. Cl. CPC H10K 85/626; C07C 15/30; C07C 15/38; C09K 11/02; H10K 85/622; H10K 85/6574; C07B 2200/05; C07C 2602/10; C07C 2603/42; H10K 50/11; H10K 2101/90

(57) ABSTRACT

The present disclosure relates to a plurality of host materials, an organic electroluminescent compound, and an organic electroluminescent device comprising the same. It is possible to provide an organic electroluminescent device having improved lifespan properties compared to a conventional organic electroluminescent device by comprising the specific combination of compounds according to the present disclosure as the plurality of host materials, or by comprising the compound according to the present disclosure.

### TECHNICAL FIELD

The present disclosure relates to an organic electroluminescent compound, a plurality of host materials, and an organic electroluminescent device comprising the same.

### BACKGROUND ART

The TPD/Alq3 bilayer small-molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. At present, OLEDs primarily use phosphorescent materials having excellent luminous efficiency in panel implementation. In many applications such as TVs and lightings, OLEDs face the problem of insufficient lifespan, and high OLED efficiency is still required. Generally, the higher the luminance of an OLED, the shorter its lifespan. Therefore, OLEDs with high luminous efficiency and/or long lifespan are required for long-term display use and high resolution.

In order to improve luminous efficiency, driving voltage, and/or lifespan, various materials or concepts for an organic layer of an organic electroluminescent device have been proposed. However, these were not satisfactory in practical use. Accordingly, there has been a continuous need to develop organic electroluminescent devices having more improved performance, for example, improved driving voltage, luminous efficiency, power efficiency, and/or lifespan properties, compared to previously disclosed organic electroluminescent devices.

Korean Patent Application Laid-Open Nos. 2008-0015953, 2006-0108642, and 2010-0121489 disclose an organic electroluminescent compound having an anthracene structure and an organic electroluminescent device using the same, or an organic light-emitting medium comprising a specific diaminopyrene derivative and a specific anthracene derivative, but do not disclose an organic electroluminescent device having improved performance by comprising a specific combination of a plurality of host materials and organic electroluminescent compounds claimed in the present disclosure.

### DISCLOSURE OF INVENTION

### Technical Problem

The object of the present disclosure is, firstly, to provide a plurality of host materials capable of producing an organic electroluminescent device having low driving voltage and/or high luminous efficiency and/or long lifespan properties, and, secondly, to provide an organic electroluminescent device comprising the plurality of host materials. Also, an object of the present disclosure is to provide an organic electroluminescent compound having a novel structure suitable for application in an organic electroluminescent device.

### Solution to Problem

As a result of intensive studies to solve the technical problems, the present inventors found that the aforementioned objects can be achieved by a plurality of host materials comprising at least one first host compound and at least one second host compound, wherein the first host compound is represented by the following Formula 1, the second host compound is represented by the following Formula 2, and the first host compound and the second host compound are different from each other, thereby completing the present invention.

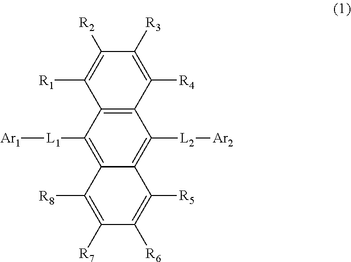

In Formula 1,
Ar₁ represents a substituted or unsubstituted (C₆-C₃₀)aryl or a substituted or unsubstituted (3- to 30-membered)heteroaryl;
L₁ represents a single bond or a substituted or unsubstituted (C₆-C₃₀)arylene;
R₁ to R₈ each independently represent hydrogen or deuterium;
L₂ represents a substituted or unsubstituted (C₆-C₃₀)arylene; and
Ar₂ is represented by the following Formula 1-1:

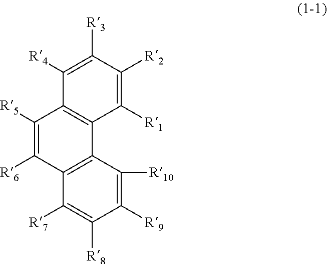

in Formula 1-1,
R′₁ to R′₁₀ each independently represent hydrogen, deuterium, or a (C₆-C₃₀)aryl unsubstituted or substituted with deuterium, and one of R′₁ to R′₁₀ is linked to L₂;

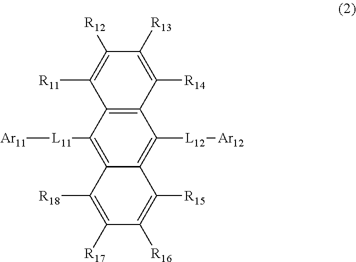

In Formula 2,
Ar₁₁ represents a substituted or unsubstituted (C₆-C₃₀)aryl or a substituted or unsubstituted (3- to 30-membered)heteroaryl;
L₁₁ represents a single bond, a substituted or unsubstituted (C₆-C₃₀)arylene, or a substituted or unsubstituted (3- to 30-membered)heteroarylene;
R₁₁ to R₁₈ each independently represent hydrogen or deuterium;
L₁₂ represents a single bond or a substituted or unsubstituted (C₆-C₃₀)arylene; and
Ar₁₂ is represented by the following Formula 2-1:

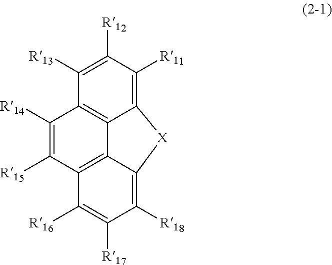

in Formula 2-1,
X is O or S;
R′₁₁ to R′₁₈ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a combination thereof; or may be linked to the adjacent substituents to form a ring(s); and
one of R′₁₁ to R′₁₈ is linked to L₁₂.

In addition, as a result of intensive studies to solve the technical problems above, the present inventors found that the aforementioned objects can be achieved by an organic electroluminescent compound represented by the following Formula 1-a, and an organic electroluminescent device comprising the same, thereby completing the present invention.

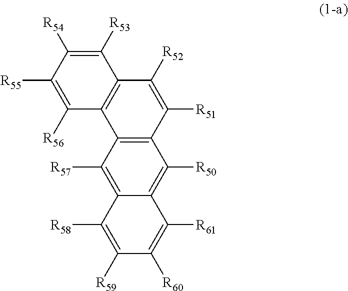

In Formula 1-a,
R₅₀ to R₆₁ each independently represent hydrogen, deuterium, the following Formula 1-b, or the following Formula 1-c, provided that at least one of R₅₀ to R₆₁ represents the following Formula 1-b or Formula 1-c:

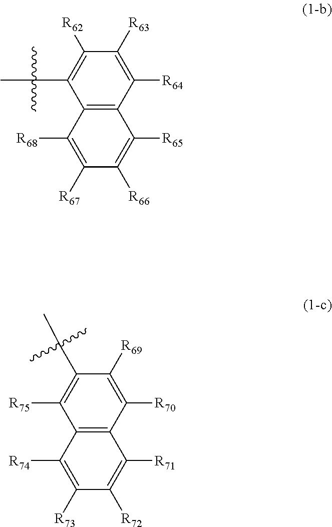

in Formulas 1-b and 1-c,
R₆₂ to R₆₅ and R₆₈ each independently represent hydrogen, deuterium, a phenyl unsubstituted or substituted with deuterium, a biphenyl unsubstituted or substituted with deuterium, or a naphthyl unsubstituted or substituted with deuterium, and at least one of R₆₂ to R₆₅ and R₆₈ represents a phenyl unsubstituted or substituted with deuterium, a biphenyl unsubstituted or substituted with deuterium, or a naphthyl unsubstituted or substituted with deuterium;
R₆₆ and R₆₇ each independently represent hydrogen or deuterium; and
R₆₉ to R₇₅ each independently represent hydrogen, deuterium, a phenyl unsubstituted or substituted with deuterium, a biphenyl unsubstituted or substituted with deuterium, or a naphthyl unsubstituted or substituted with deuterium, and at least one of R₆₉ to R₇₅ represents a phenyl unsubstituted or substituted with deuterium, a biphenyl unsubstituted or substituted with deuterium, or a naphthyl unsubstituted or substituted with deuterium.

Furthermore, as a result of intensive studies to solve the technical problems above, the present inventors found that the aforementioned objects can be achieved by an organic electroluminescent compound represented by the following Formula 2-a, and an organic electroluminescent device comprising the same, thereby completing the present invention.

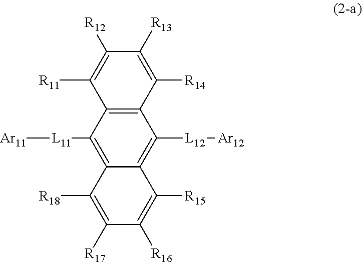

In Formula 2-a,
Ar₁₁ represents a substituted or unsubstituted (C₆-C₃₀)aryl or a substituted or unsubstituted (3- to 30-membered)heteroaryl;
L₁₁ represents a single bond, a substituted or unsubstituted (C₆-C₃₀)arylene, or a substituted or unsubstituted (3- to 30-membered)heteroarylene;
R₁₁ to R₁₈ each independently represent hydrogen or deuterium;
L₁₂ represents a single bond or a substituted or unsubstituted (C₆-C₃₀)arylene; and
Ar₁₂ is represented by the following Formula 2-b:

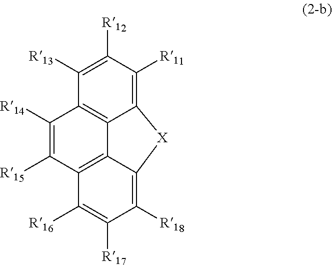

in Formula 2-b,
X is O or S;
R′₁₁ to R′₁₈ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a combination thereof; or may be linked to the adjacent substituents to form a ring(s);
one of R′₁₁ to R′₁₃ is linked to L₁₂; and
at least one of the remainder of R′₁₁ to R′₁₃ not linked to L₁₂ and R′₁₄ to R′₁₈ is a substituted or unsubstituted (C₁-C₃₀)alkyl, a substituted or unsubstituted (C₃-C₃₀)cycloalkyl, a substituted or unsubstituted (C₆-C₃₀)aryl, a substituted or unsubstituted (C₆-C₃₀)heteroaryl, or a combination thereof.

### Advantageous Effects of Invention

By using a plurality of host materials or an organic electroluminescent compound according to the present disclosure, an organic electroluminescent device with low driving voltage and/or high luminous efficiency and/or long lifespan properties can be produced.

### MODE FOR THE INVENTION

Hereinafter, the present disclosure will be described in detail. However, the following description is intended to explain the present disclosure, and is not meant in any way to restrict the scope of the present disclosure.

The “organic electroluminescent compound” in the present disclosure refers to a compound that may be used in an organic electroluminescent device, and may be included in any layer constituting an organic electroluminescent device, as necessary.

The “organic electroluminescent material” in the present disclosure refers to a material that may be used in an organic electroluminescent device, and may comprise at least one compound. The organic electroluminescent material may be included in any layer constituting an organic electroluminescent device, as necessary. For example, the organic electroluminescent material may be a hole injection material, a hole transport material, a hole auxiliary material, a light-emitting auxiliary material, an electron-blocking material, a light-emitting material (comprising a host material and a dopant material), an electron buffer material, a hole-blocking material, an electron transport material, an electron injection material, etc.

The “plurality of host materials” in the present disclosure refers to a host material comprising a combination of at least two compounds that may be included in any light-emitting layer constituting an organic electroluminescent device. It may mean both a material before being included in an organic electroluminescent device (e.g., before vapor deposition) and a material after being included in an organic electroluminescent device (e.g., after vapor deposition). For example, the plurality of host materials of the present disclosure may be a combination of at least two host materials, and may optionally further comprise conventional materials comprised in an organic electroluminescent material. At least two compounds comprised in the plurality of host materials of the present disclosure may be included together in one light-emitting layer, or may respectively be included in different light-emitting layers. For example, the at least two host materials may be mixture-evaporated or co-evaporated, or may be individually evaporated.

Herein, the “(C₁-C₃₀)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The “(C₃-C₃₀)cycloalkyl” is meant to be a mono- or polycyclic hydrocarbon having 3 to 30 ring skeleton carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The “(3- to 7-membered)heterocycloalkyl” is meant to be a cycloalkyl having 3 to 7 ring skeleton atoms, comprising at least one heteroatom selected from the group consisting of B, N, O, S, Si, and P, preferably the group consisting of O, S, and N. The above heterocycloalkyl may include tetrahydrofuran, pyrrolidine, thiolane, tetrahydropyran, etc. The “(C₆-C₃₀)aryl” and “(C₆-C₃₀)arylene” are meant to be a monocyclic or fused ring-type radical derived from an aromatic hydrocarbon having 6 to 30 ring skeleton carbon atoms, and may be partially saturated. The above aryl, arylene, and arenetriyl may comprise those having a spiro structure. The above aryl may include phenyl, biphenyl, terphenyl, quinquephenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopentene-fluoren]yl, spiro[dihydroindene-fluoren]yl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the “(3- to 30-membered)heteroaryl” and “(3- to 30-membered)heteroarylene” are meant to be an aryl or arylene group having 3 to 30 ring skeleton atoms, comprising at least one heteroatom(s) selected from the group consisting of B, N, O, S, Si, P, Se, Te, and Ge. The number of heteroatoms is preferably 1 to 4. The above heteroaryl or heteroarylene may be a monocyclic ring or a fused ring condensed with at least one benzene ring, and may be partially saturated. In addition, the above heteroaryl or heteroarylene may be one formed by linking at least one heteroaryl or aryl group to a heteroaryl group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, naphthooxazolyl, benzofuroquinolinyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, phenanthrooxazolyl, phenanthrothiazolyl, phenanthrobenzofuranyl, benzophenanthrothiophenyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl,3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The “(3- to 30-membered)heteroaryl(ene)” can be classified into a heteroaryl(ene) with electronic properties and a heteroaryl(ene) with hole properties. The heteroaryl(ene) with electronic properties is a substituent that is relatively rich in electrons in the parent nucleus, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, or a substituted or unsubstituted quinolyl. The heteroaryl(ene) with hole properties is a substituent that is relatively electron-deficient in the parent nucleus, for example, a substituted or unsubstituted carbazolyl, a substituted or unsubstituted dibenzofuranyl, or a substituted or unsubstituted dibenzothiophenyl. In the present disclosure, the “halogen” includes F, Cl, Br, and I.

In addition, “ortho-” (“o-”), “meta-” (“m-”), and “para-” (“p-”) are prefixes which each represent the relative positions of substituents. The prefix “ortho-” indicates that two substituents are adjacent to each other, and for example, when two substituents in a benzene derivative occupy positions 1 and 2 or positions 2 and 3, this is called an “ortho-” configuration. The prefix “meta-” indicates that two substituents are at positions 1 and 3, and for example, when two substituents in a benzene derivative occupy positions 1 and 3, this is called a “meta-” configuration. The prefix “para-” indicates that two substituents are at positions 1 and 4, and for example, when two substituents in a benzene derivative occupy positions 1 and 4, this is called a “para-” configuration.

Herein, “substituted” in the expression “substituted or unsubstituted” means that a hydrogen atom in a certain functional group is replaced with another atom or another functional group, i.e., a substituent. Unless otherwise specified, the substituent may replace hydrogen at a position where the substituent can be substituted without limitation, and when two or more hydrogen atoms in a certain functional group are each replaced with a substituent, each substituent may be the same as or different from one another. The maximum number of substituents that can be substituted for a certain functional group may be the total number of valences that can be substituted for each atom forming the functional group. Herein, the substituted alkyl, the substituted aryl, the substituted arylene, the substituted heteroaryl, the substituted heteroarylene, the substituted cycloalkyl, the substituted fused ring group of an aliphatic ring(s) and an aromatic ring(s), the substituted arene, and the substituted heteroarene may each independently be substituted with at least one selected from the group consisting of deuterium, a halogen, a cyano, carboxyl, nitro, hydroxyl, phosphine oxide group, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (3- to 30-membered)heteroaryl, (C₆-C₃₀)aryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, amino, mono- or di(C₁-C₃₀)alkylamino, mono- or di(C₂-C₃₀)alkenylamino, mono- or di(C₆-C₃₀)arylamino, mono- or di(3- to 30-membered)heteroarylamino, (C₁-C₃₀)alkyl(C₂-C₃₀)alkenylamino, (C₁-C₃₀)alkyl(C₆-C₃₀)arylamino, (C₁-C₃₀)alkyl(3- to 30-membered)heteroarylamino, (C₂-C₃₀)alkenyl(C₆-C₃₀)arylamino, (C₂-C₃₀)alkenyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, di(C₆-C₃₀)arylboronyl, (C₆-C₃₀)arylphosphinyl, di(C₁-C₃₀)alkylboronyl, (C₁-C₃₀)alkyl(C₆-C₃₀)arylboronyl, (C₆-C₃₀)ar(C₁-C₃₀)alkyl, (C₁-C₃₀)alkyl(C₆-C₃₀)aryl, and a combination thereof, each of which may be further substituted with deuterium. According to one embodiment of the present disclosure, the substituted alkyl, etc. may each independently be substituted with at least one selected from the group consisting of deuterium, (C₁-C₃₀)alkyl, a substituted or unsubstituted (3- to 30-membered)heteroaryl, and a substituted or unsubstituted (C₆-C₃₀)aryl. According to another embodiment of the present disclosure, the substituted alkyl, etc. may each independentlybe substituted with at least one selected from the group consisting of deuterium, a substituted or unsubstituted (3- to 20-membered)heteroaryl, and a substituted or unsubstituted (C₆-C₂₀)aryl. For example, the substituted alkyl, etc. may each independently be substituted with at least one selected from the group consisting of deuterium, phenyl, naphthyl, phenanthrenyl, biphenyl, dimethylfluorenyl, dibenzofuranyl, dibenzothiophenyl, carbazolyl, and benzocarbazolyl, each of which may be further substituted with deuterium.

Herein, if a substituent is not indicated in the formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In cases where a substituent is not indicated in the formula or compound structure in the present disclosure, if the substituent is not explicitly excluded, such as 0% deuterium, 100% hydrogen, and all substituents being hydrogen, hydrogen and deuterium may be used together in a compound. Deuterium is one of the isotopes of hydrogen and an element with a deuteron, consisting of one proton and one neutron, as its nucleus. It can be represented as hydrogen-2, whose element symbol can also be written as D or ²H. Isotopes are atoms with the same atomic number (Z) but different mass numbers (A), and can also be interpreted as elements with the same number of protons but different a number of neutrons.

Herein, “a combination thereof” refers to a combination of one or more elements from the corresponding list to form a known or chemically stable arrangement that can be envisioned by one skilled in the art from the corresponding list. For example, alkyl and deuterium can be combined to form a partially or fully deuterated alkyl group; halogen and alkyl can be combined to form a halogenated alkyl substituent; and halogen, alkyl, and aryl can be combined to form a halogenated arylalkyl. For example, a preferred combination of substituents comprises up to 50 atoms excluding hydrogen or deuterium, up to 40 atoms excluding hydrogen or deuterium, up to 30 atoms excluding hydrogen or deuterium, or in many cases, a preferred combination of substituents may comprise up to 20 atoms excluding hydrogen or deuterium.

In the formula of the present disclosure, when forming a ring by linked to adjacent substituents, the ring may be linked to adjacent two or more substituents to form a substituted or unsubstituted, mono- or polycyclic, (3- to 30-membered) alicyclic or aromatic ring, or a combination thereof. In addition, the formed ring may comprise at least one heteroatom selected from B, N, O, S, Si, and P, preferably at least one heteroatom selected from N, O, and S. According to one embodiment of the present disclosure, the number of ring skeleton atoms is 5 to 20, and according to another embodiment of the present disclosure, the number of ring skeleton atoms is 5 to 15.

The present disclosure provides a plurality of host materials comprising at least one first host compound and at least one second host compound, wherein the first host compound is represented by the following Formula 1, the second host compound is represented by the following Formula 2, and the first host compound and the second host compound are different from each other.

The compound represented by Formula 1 is described in more detail as follows.

In Formula 1, Ar₁ represents a substituted or unsubstituted (C₆-C₃₀)aryl, or a substituted or unsubstituted (3- to 30-membered)heteroaryl. According to one embodiment of the present disclosure, Ar₁ may be a substituted or unsubstituted (C₆-C₂₀)aryl, or a substituted or unsubstituted (3- to 15-membered)heteroaryl. According to another embodiment of the present disclosure, Ar₁ may be a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof. According to yet another embodiment of the present disclosure, Ar₁ may be a substituted or unsubstituted (C₆-C₁₄)aryl. For example, Ar₁ may be a phenyl, a naphthyl, a phenanthrenyl, or a biphenyl, etc., which may be further substituted with deuterium.

L₁ represents a single bond or a substituted or unsubstituted (C₆-C₃₀)arylene. According to one embodiment of the present disclosure, L₁ may be a single bond or a substituted or unsubstituted (C₆-C₂₀)arylene. According to another embodiment of the present disclosure, L₁ may be a single bond, a phenylene unsubstituted or substituted with deuterium, a biphenylene unsubstituted or substituted with deuterium, a terphenylene unsubstituted or substituted with deuterium, or a naphthylene unsubstituted or substituted with deuterium. According to yet another embodiment of the present disclosure, L₁ may be a (C₆-C₁₂)arylene. For example, L₁ may be a single bond or a phenylene, etc., which may be further substituted with deuterium.

R₁ to R₈ each independently represent hydrogen or deuterium.

L₂ represents a substituted or unsubstituted (C₆-C₃₀)arylene. According to one embodiment of the present disclosure, L₂ may be a substituted or unsubstituted (C₆-C₂₀)arylene. According to another embodiment of the present disclosure, L₂ may be a single bond, a phenylene unsubstituted or substituted with deuterium, a biphenylene unsubstituted or substituted with deuterium, a terphenylene unsubstituted or substituted with deuterium, or a naphthylene unsubstituted or substituted with deuterium. According to yet another embodiment of the present disclosure, L₂ may be a (C₆-C₁₂)arylene. For example, L₂ may be a phenylene, a naphthylene, or a biphenylene, etc., which may be further substituted with deuterium.

Ar₂ is represented by the following Formula 1-1.

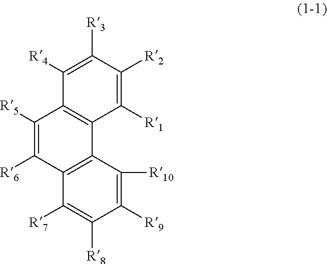

In Formula 1-1, R′₁ to R′₁₀ each independently represent hydrogen, deuterium, or a (C₆-C₃₀)aryl unsubstituted or substituted with deuterium, and one of R′₁ to R′₁₀ is linked to L₂. According to one embodiment of the present disclosure, R′₁ to R′₁₀ may each independently be hydrogen, deuterium, or a (C₆-C₂₀)aryl unsubstituted or substituted with deuterium, and one of R′₁ to R′₁₀ may be linked to L₂. According to another embodiment of the present disclosure, R′₁ to R′₁₀ may each independently be hydrogen, deuterium, a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof. According to yet another embodiment of the present disclosure, R′₁ to R′₁₀ may each independently be a (C₆-C₁₂)aryl. For example, R′₁ to R′₁₀ may each independently be hydrogen, deuterium, or a phenyl, etc., which may be further substituted with deuterium.

According to one embodiment of the present disclosure, the compound represented by Formula 1 may be at least one selected from the following compounds, but is not limited thereto.

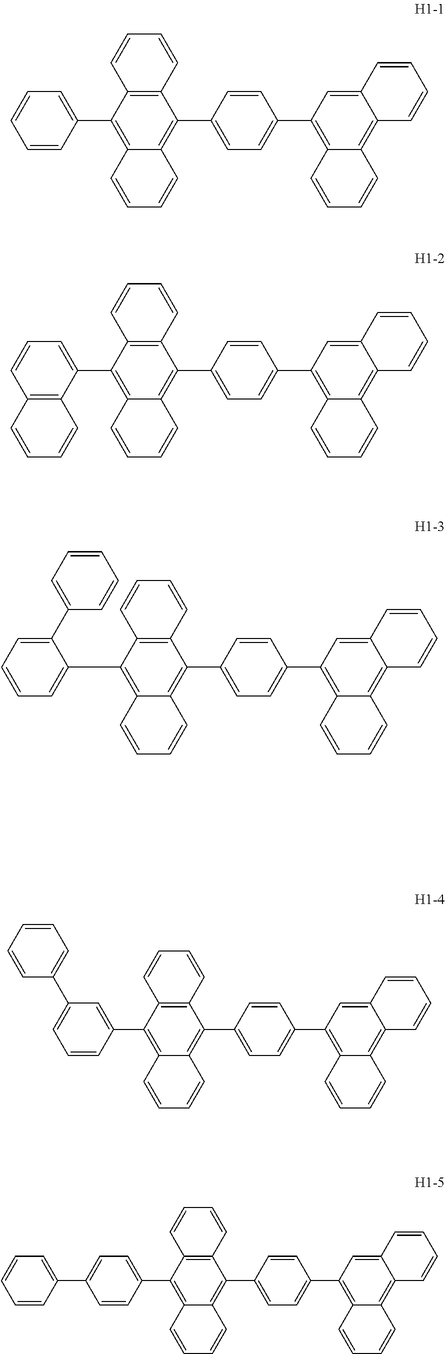

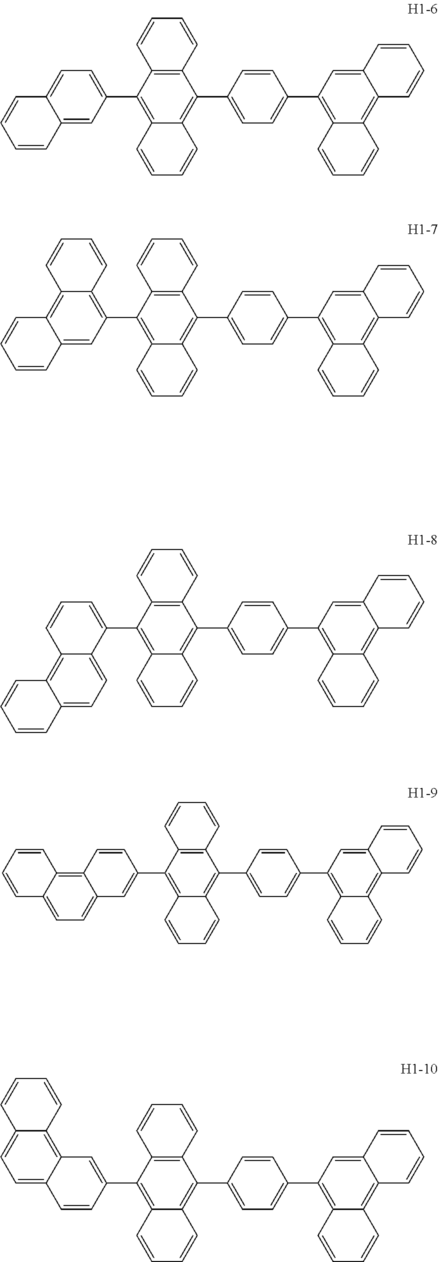

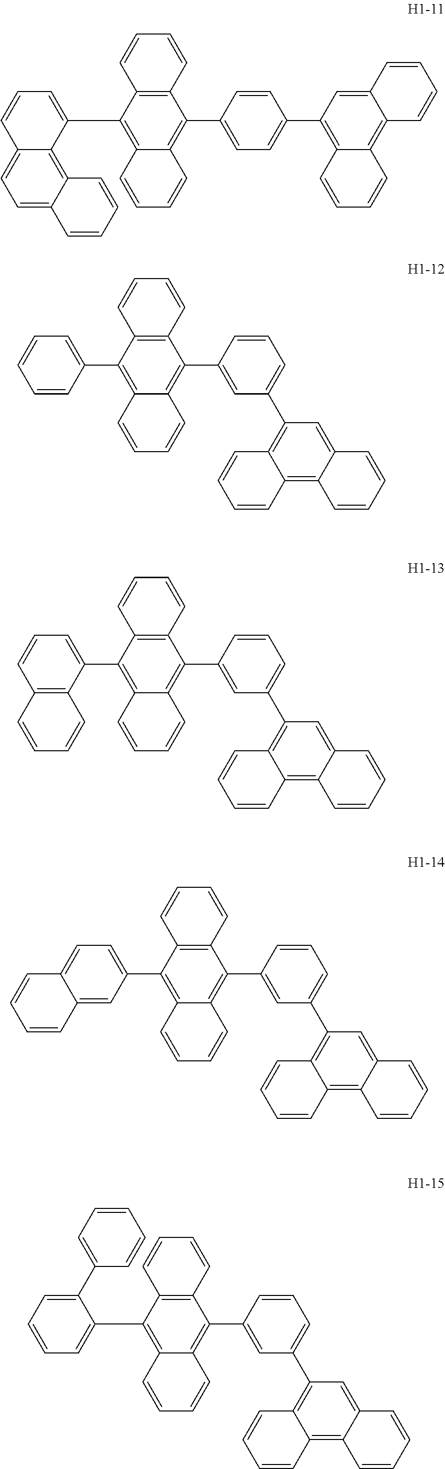

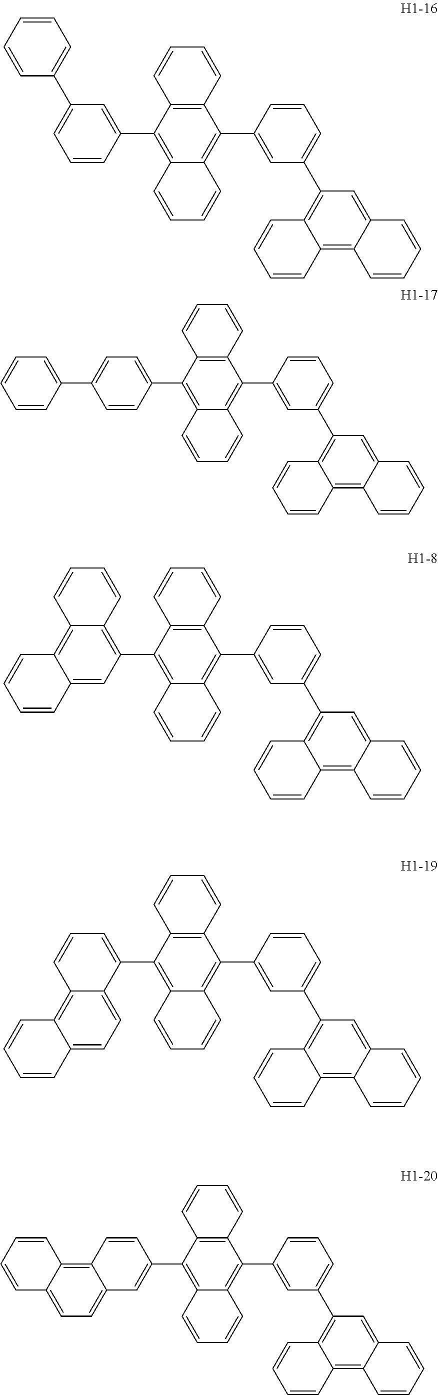

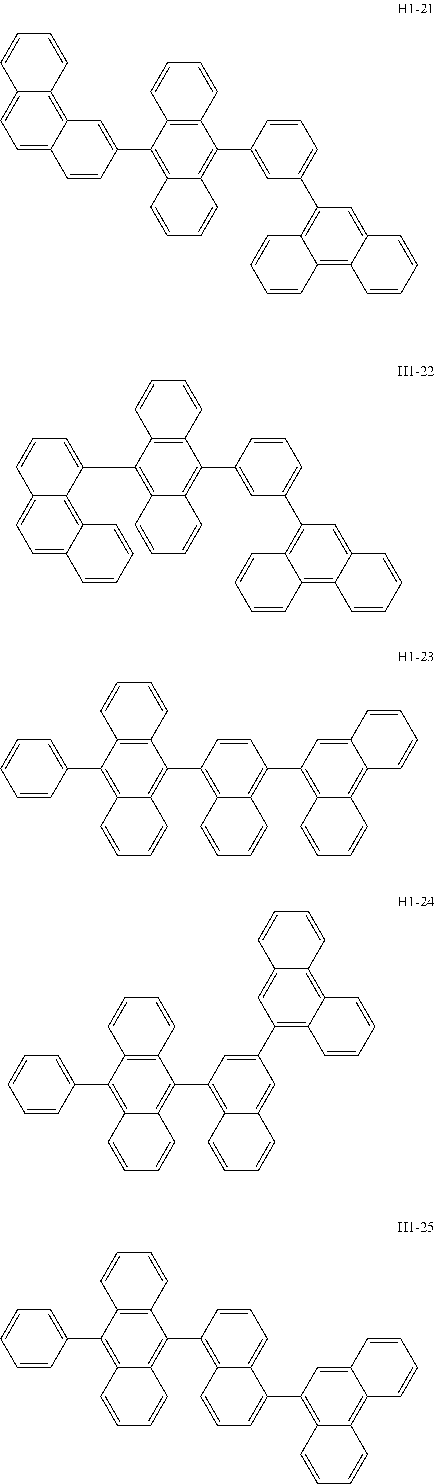

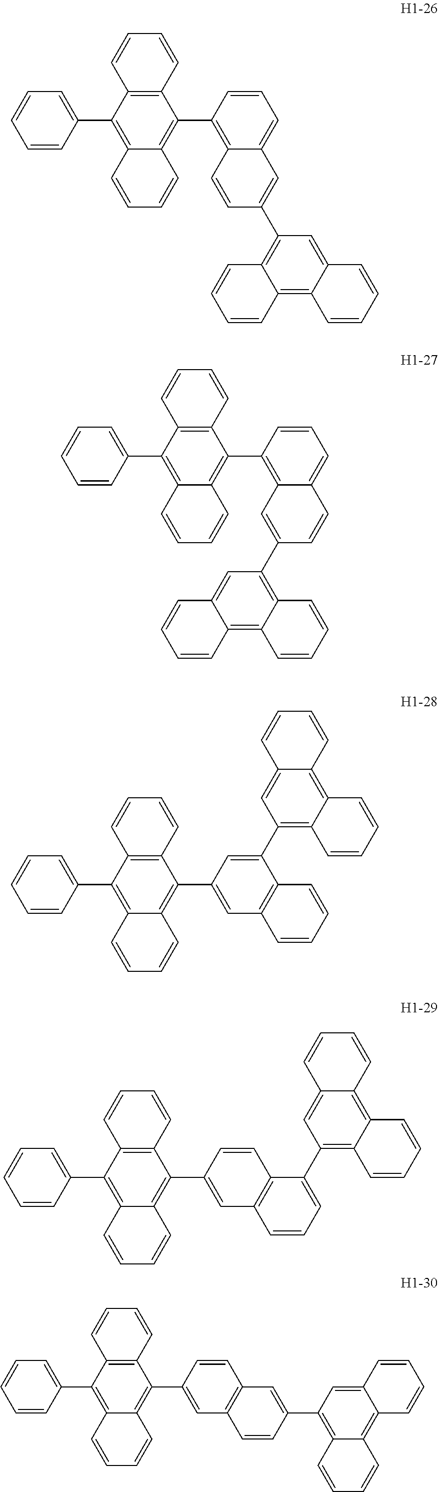

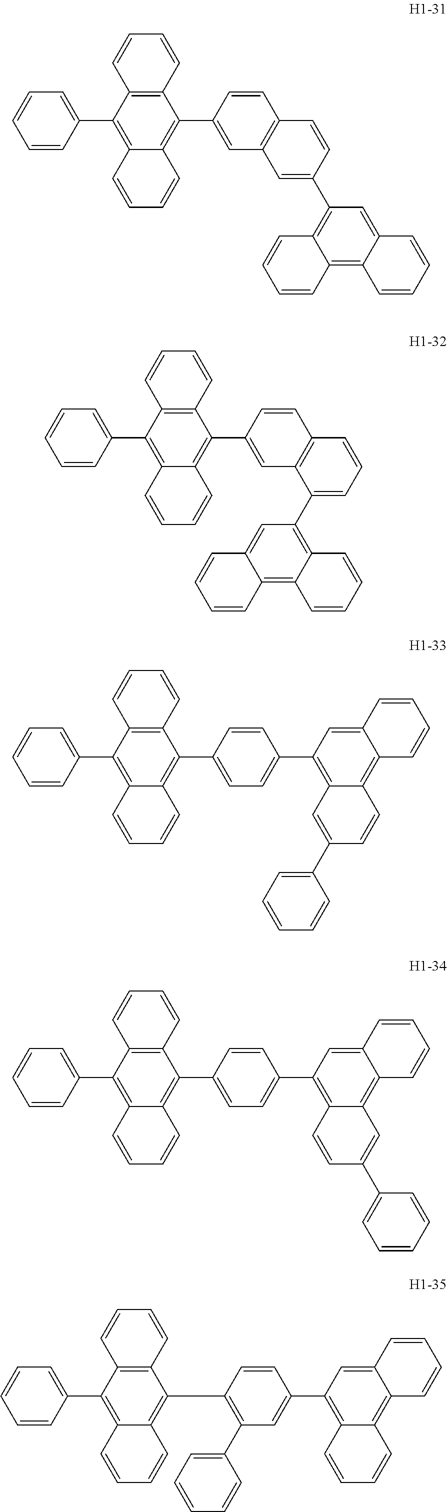

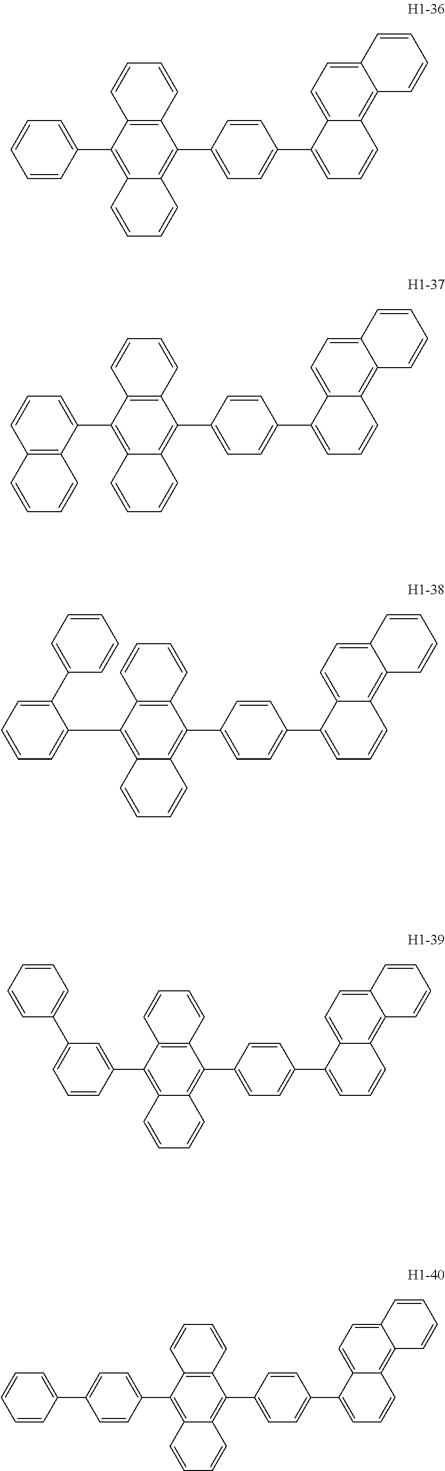

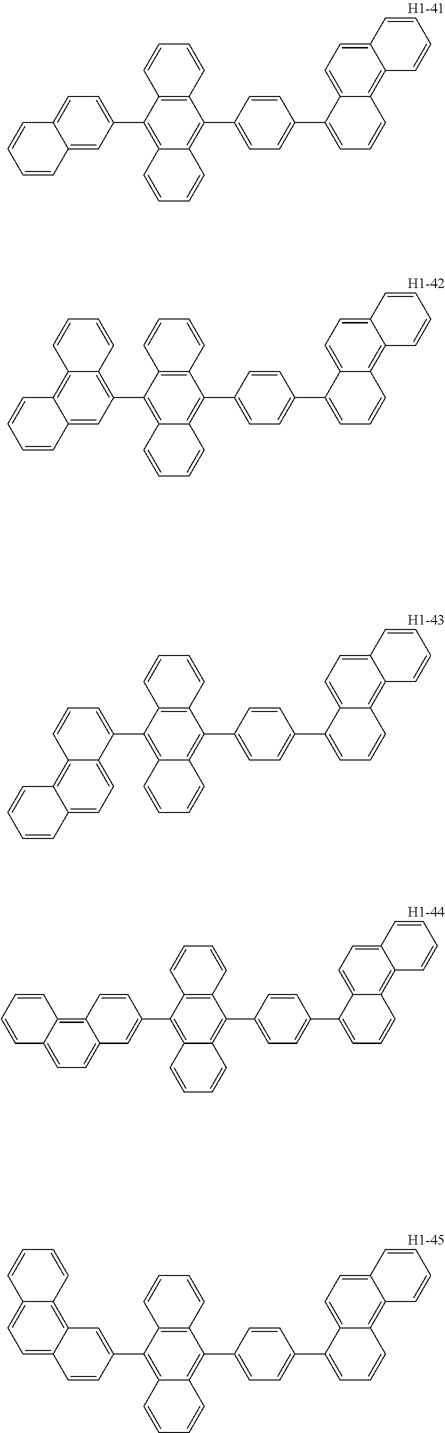

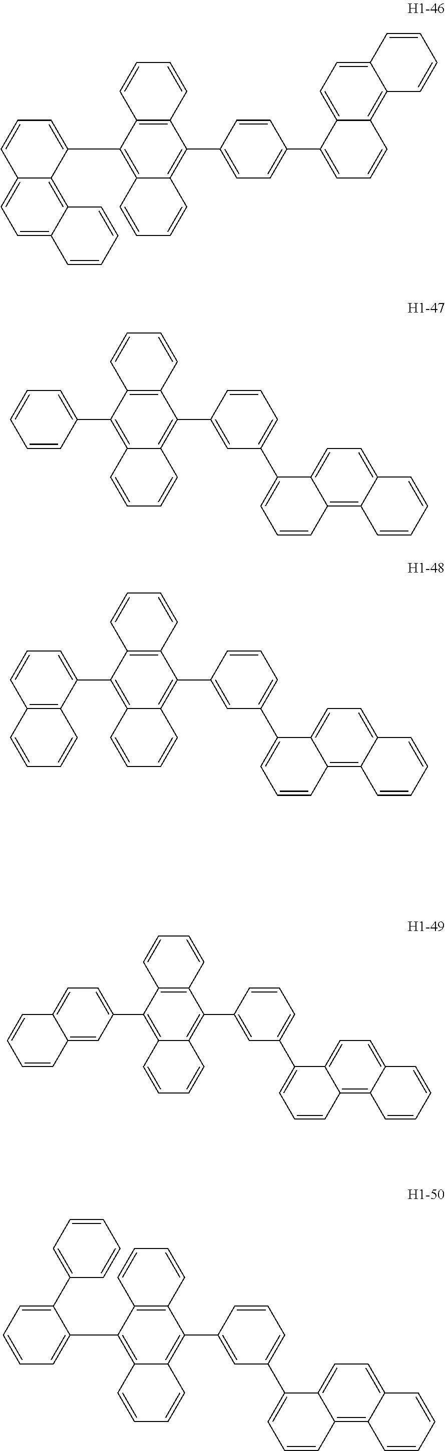

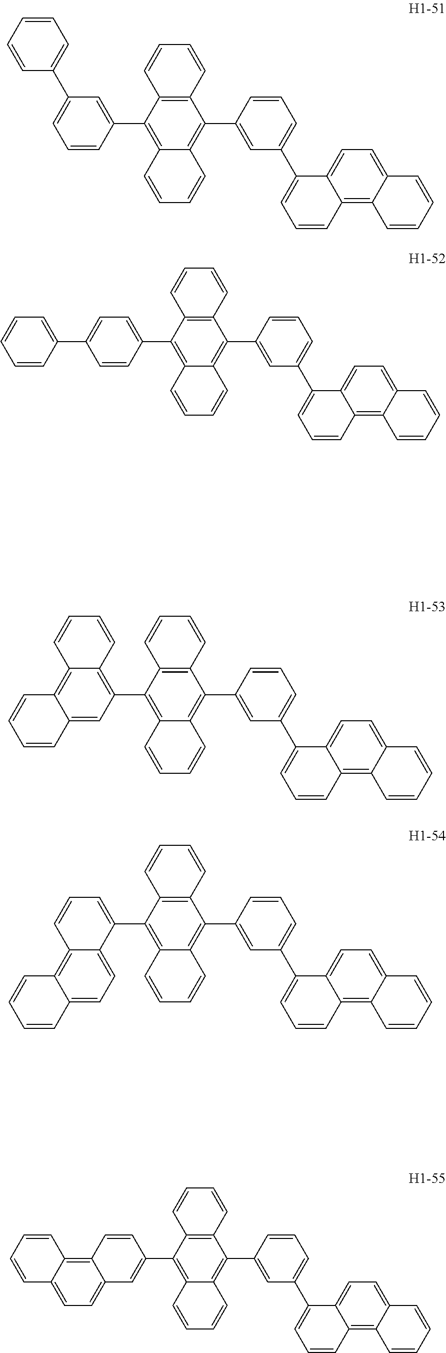

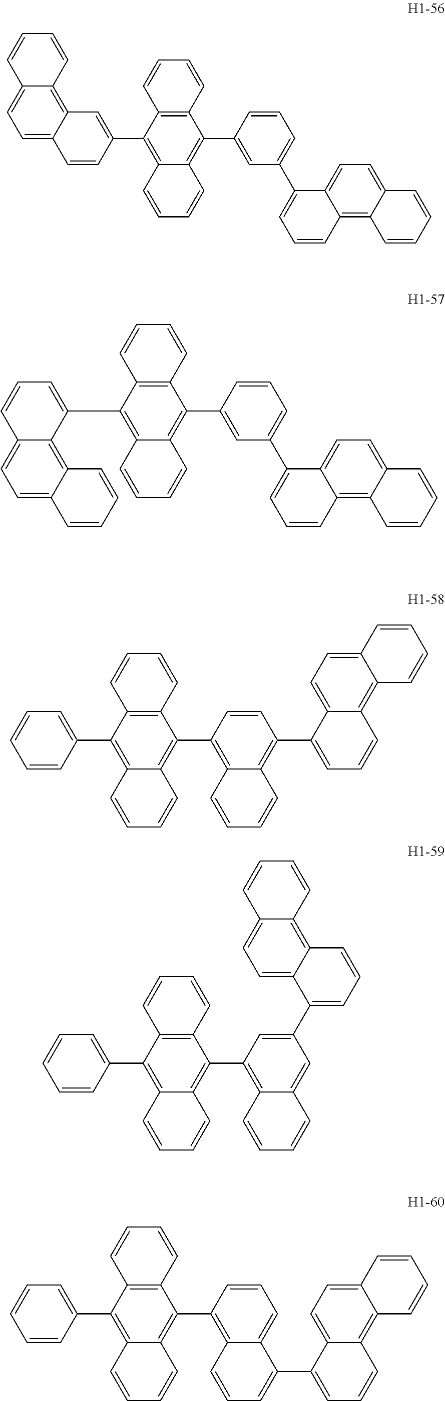

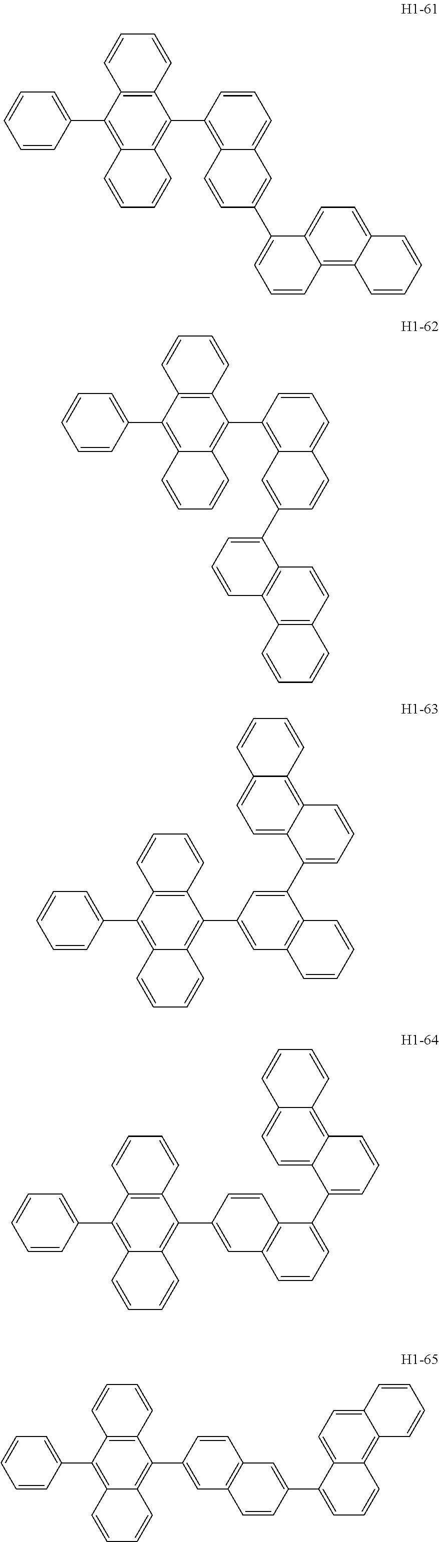

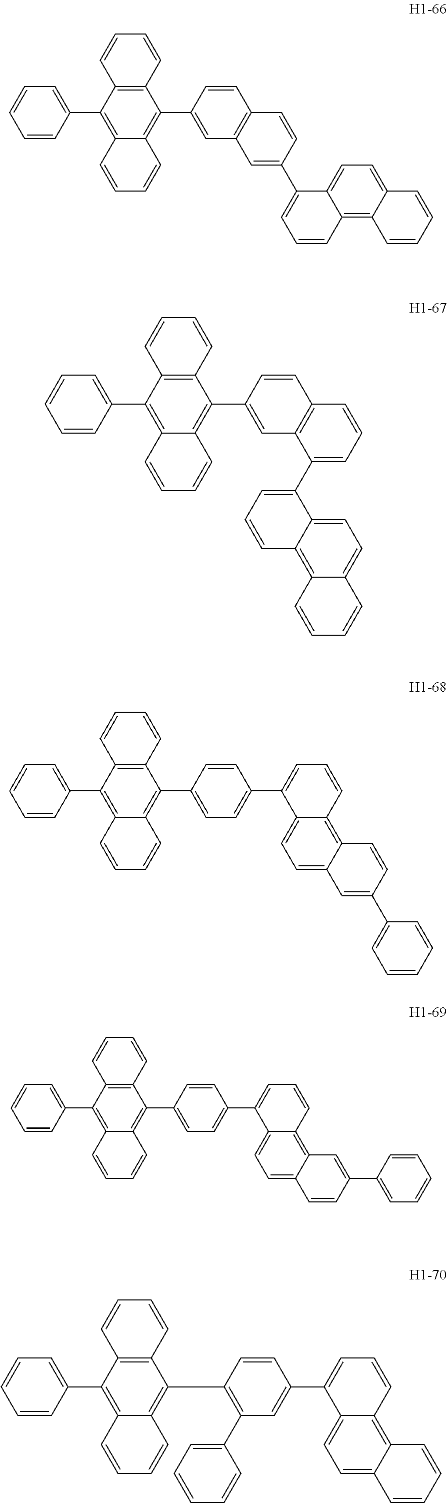

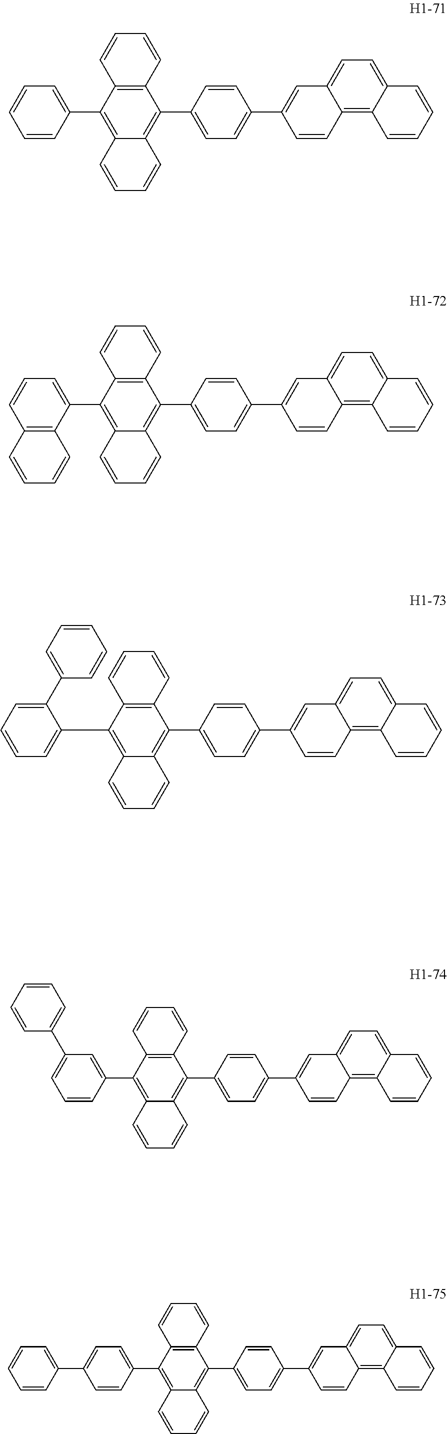

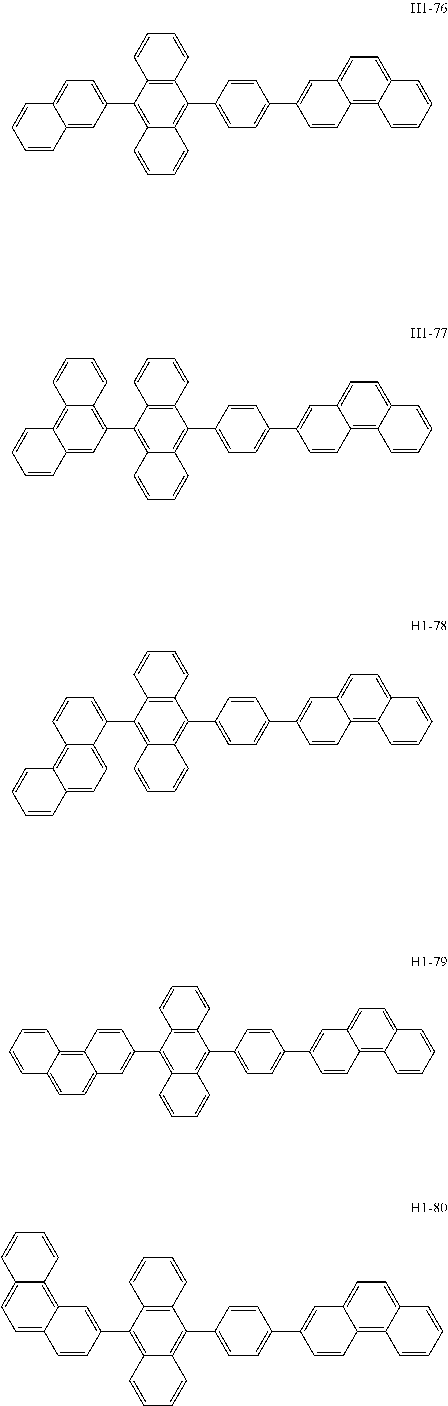

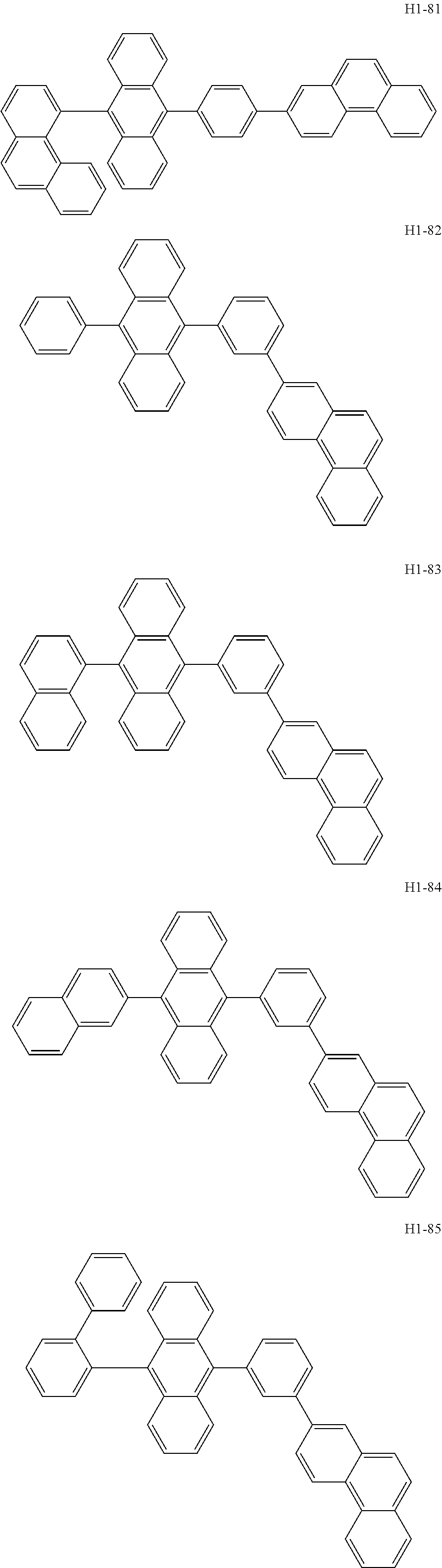

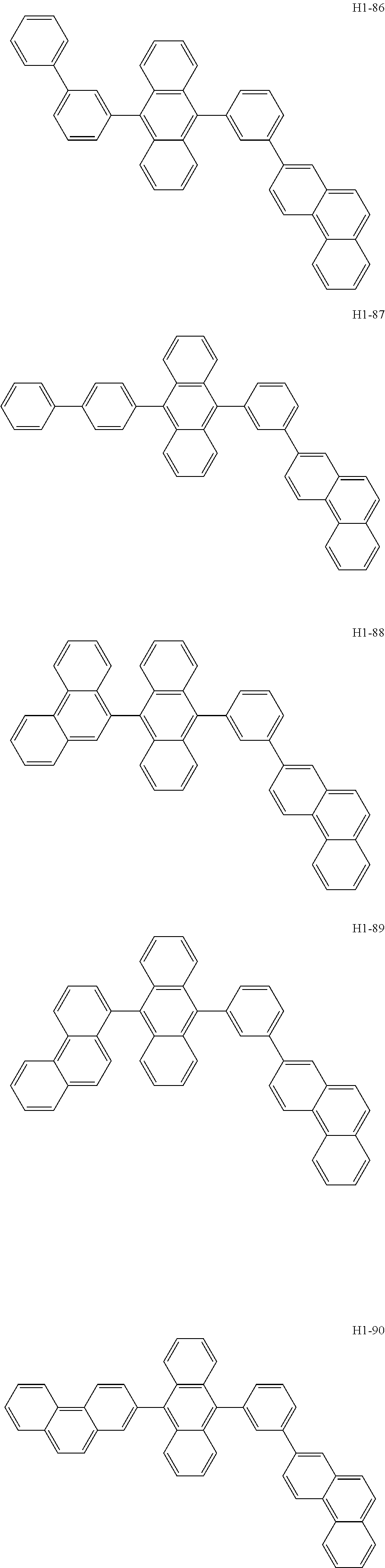

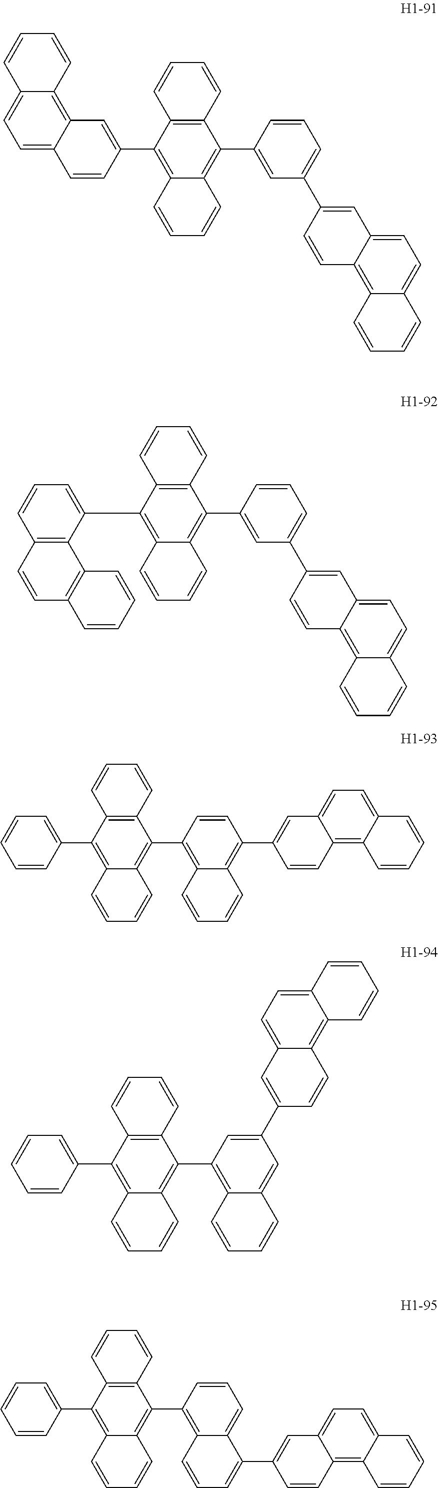

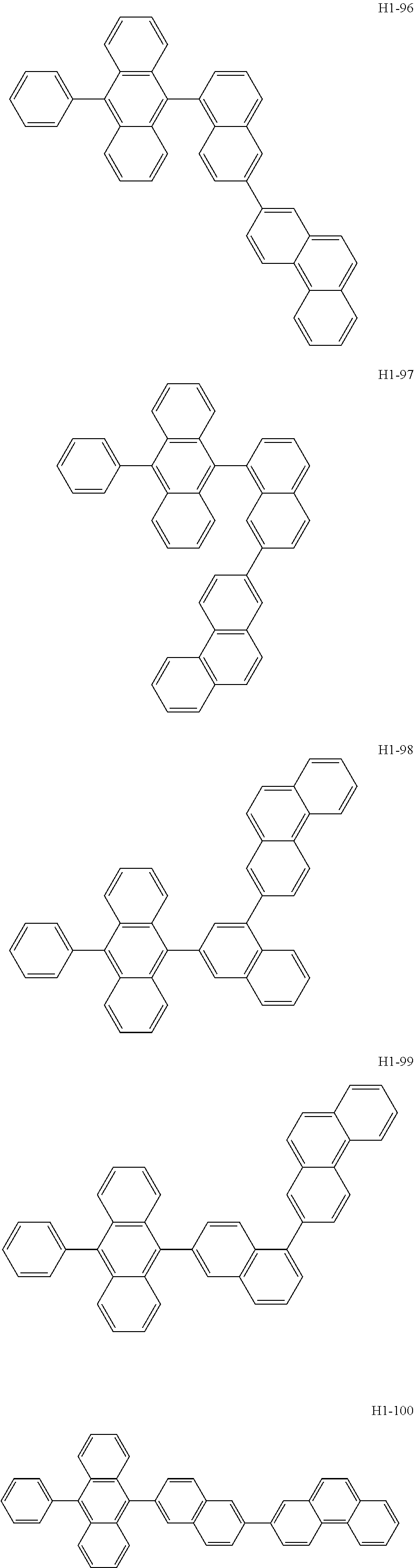

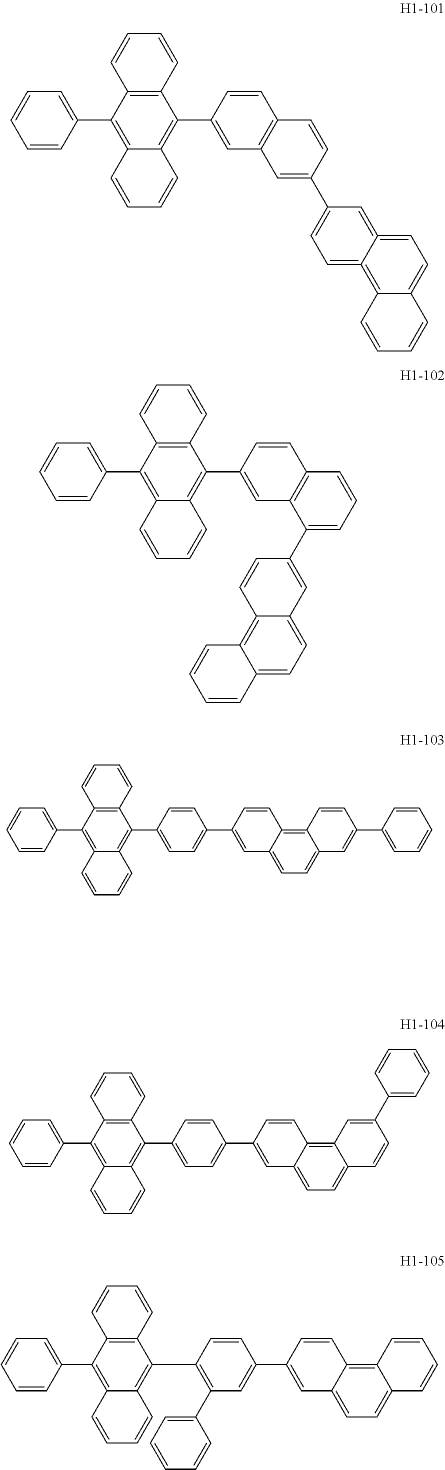

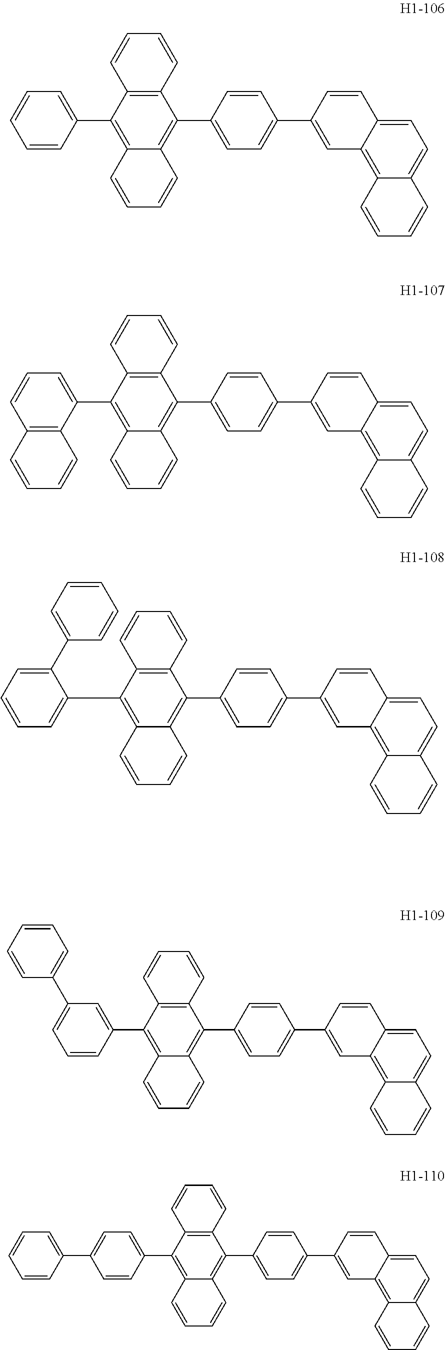

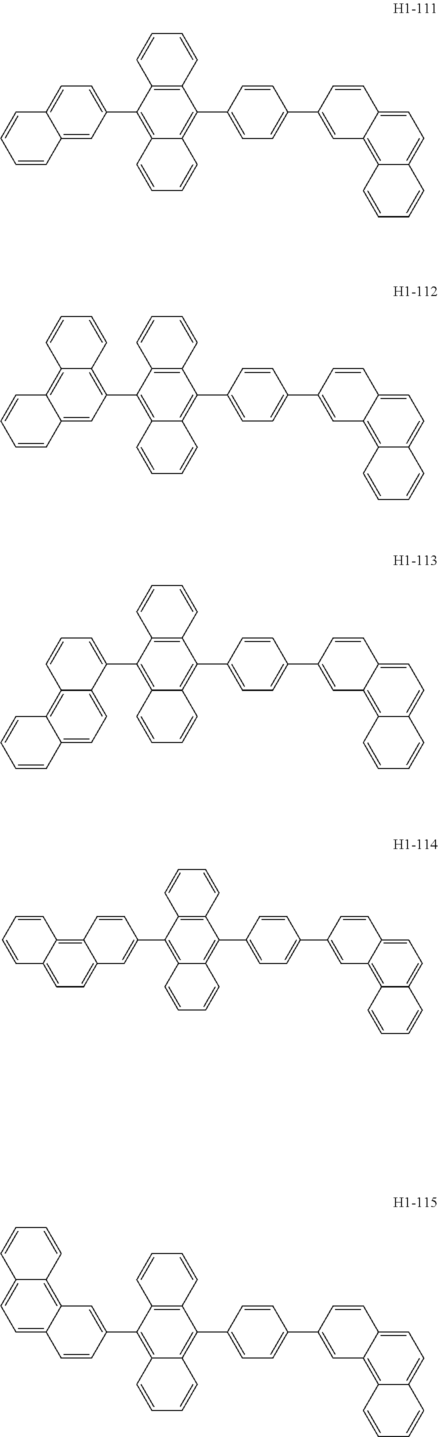

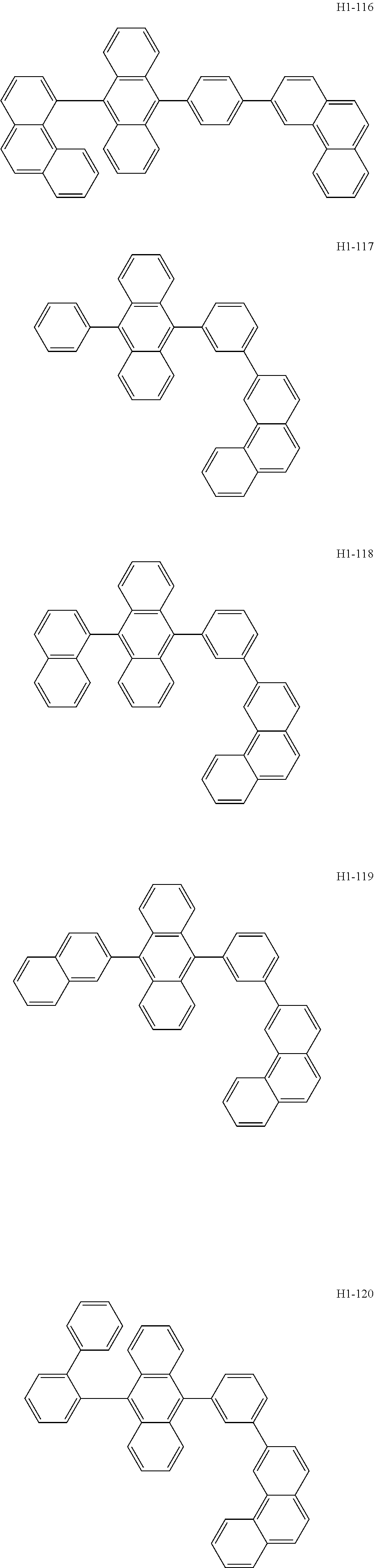

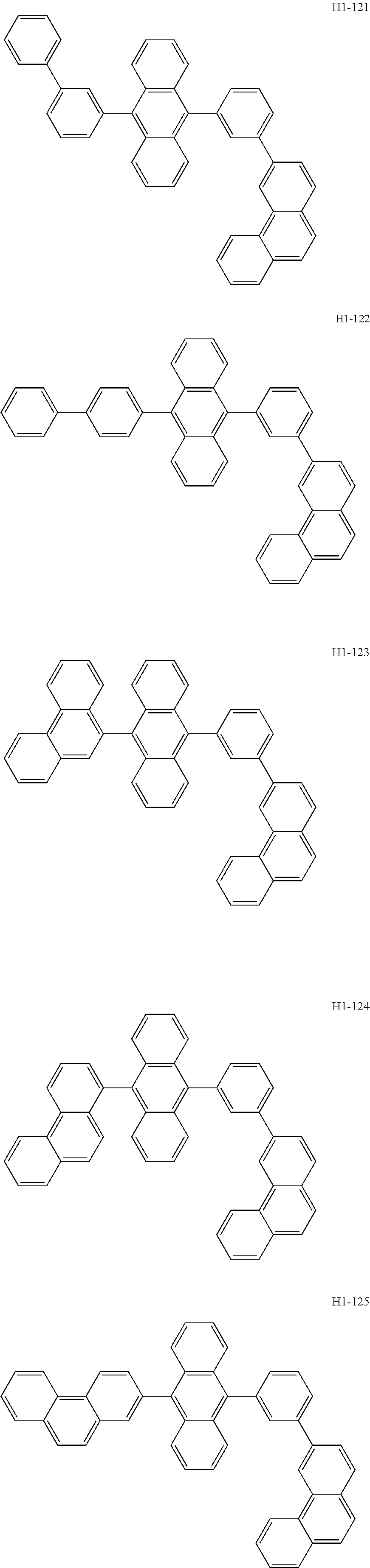

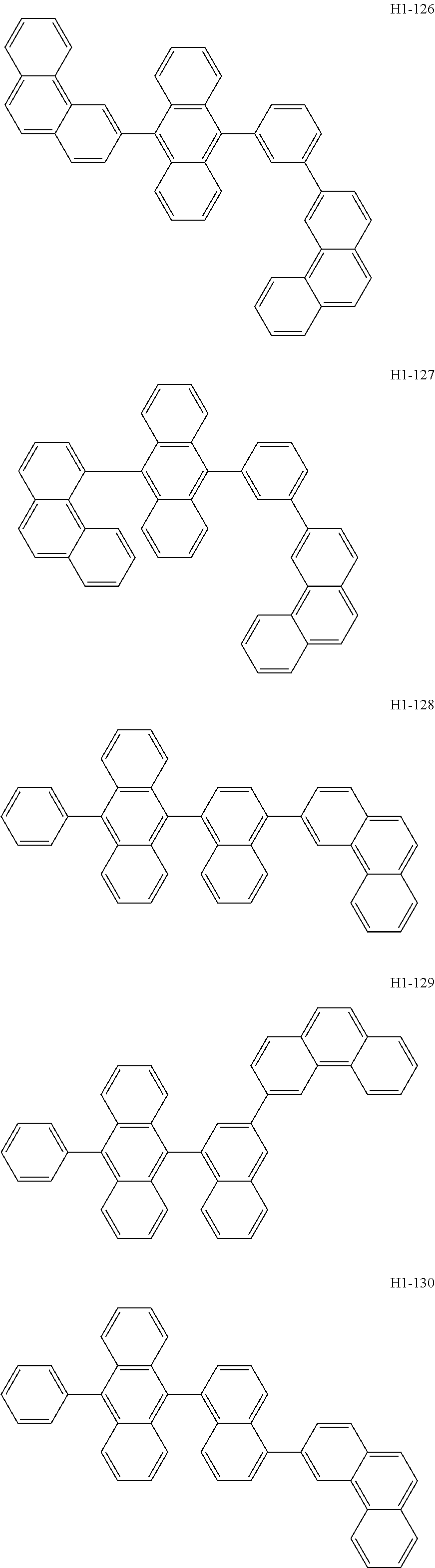

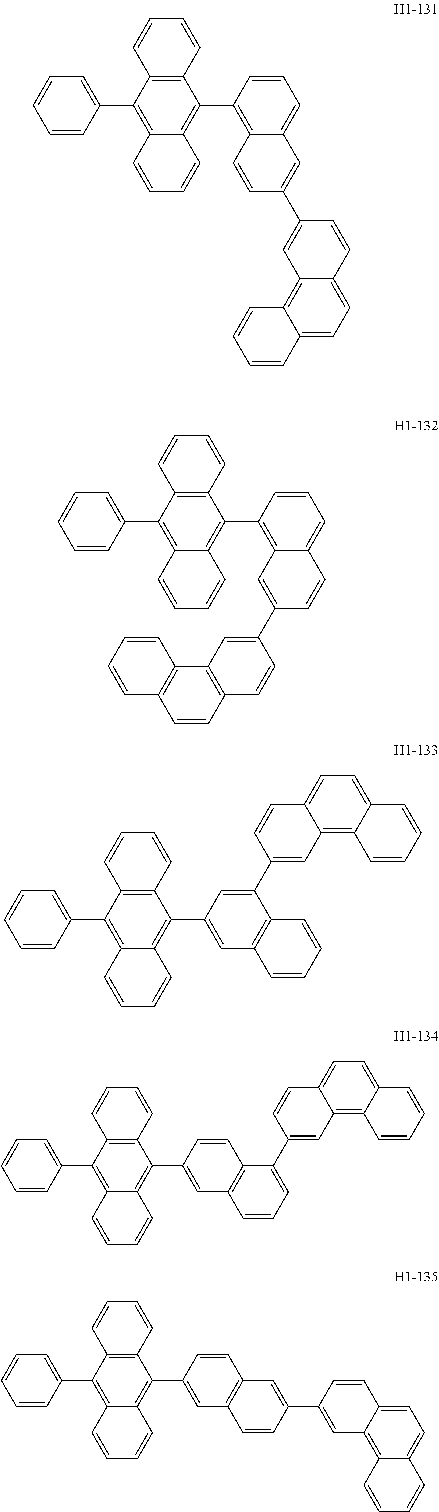

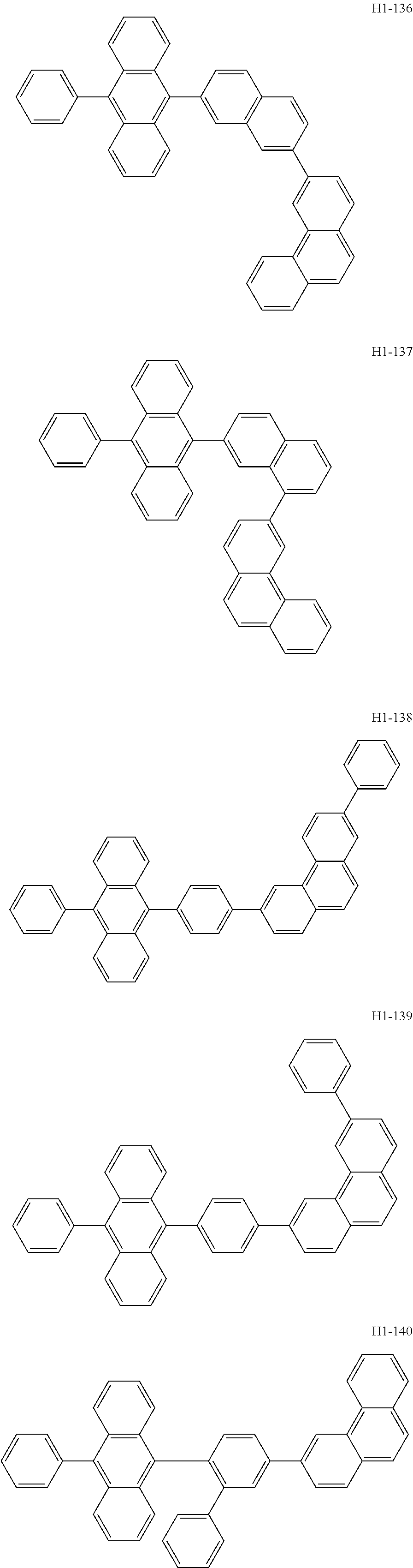

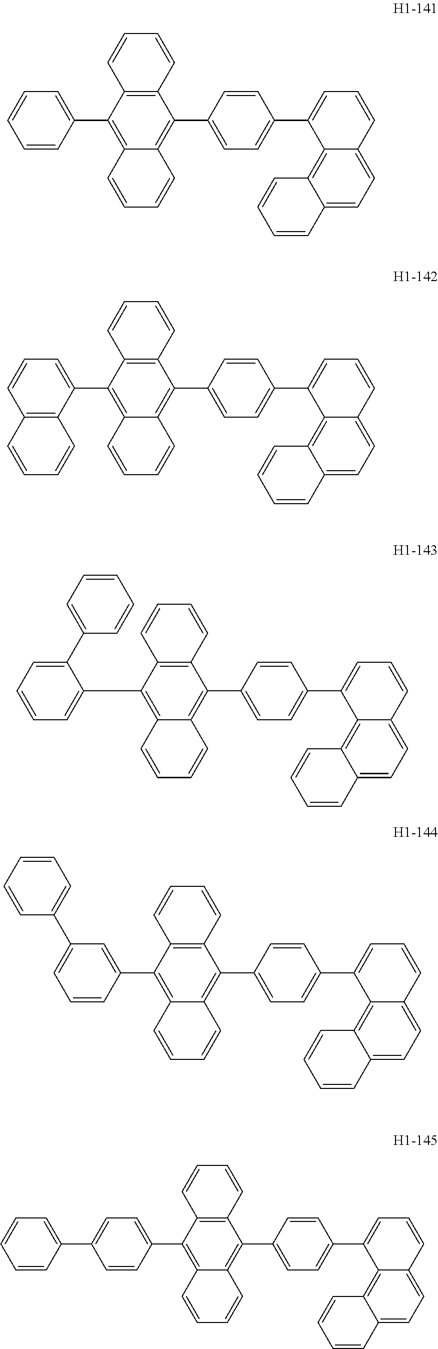

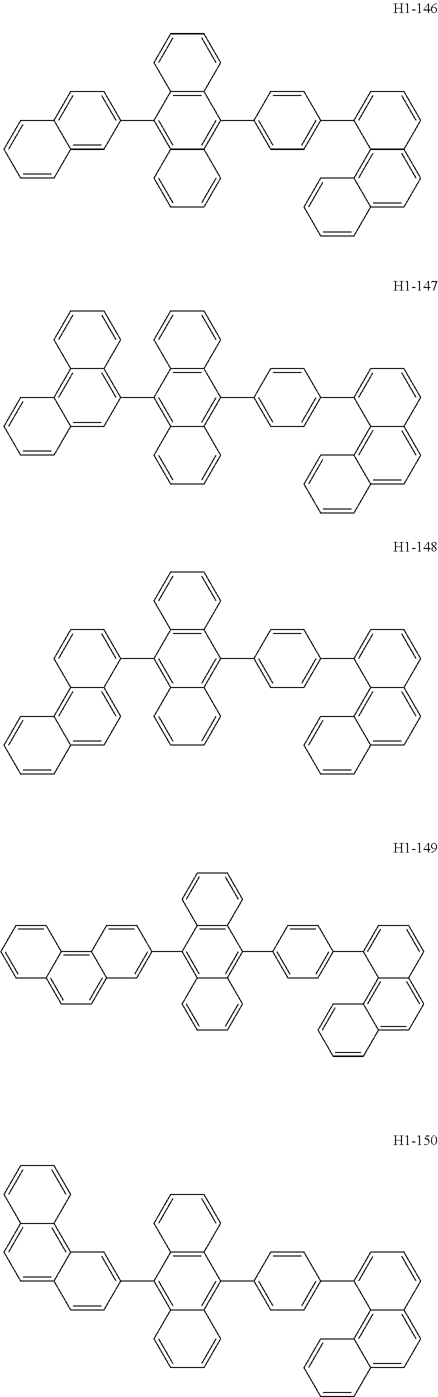

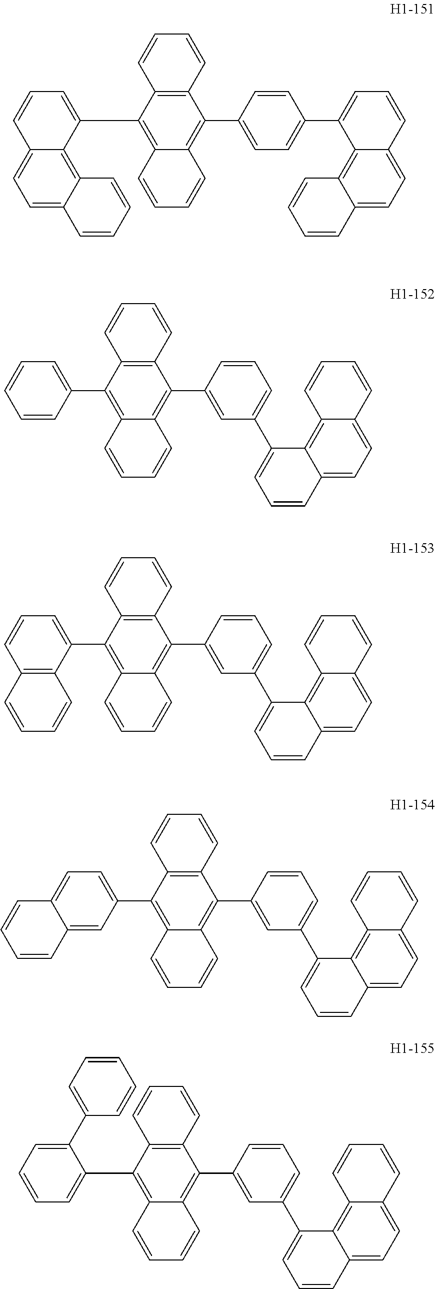

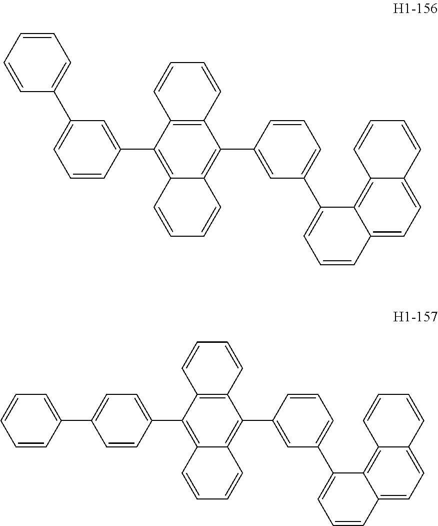

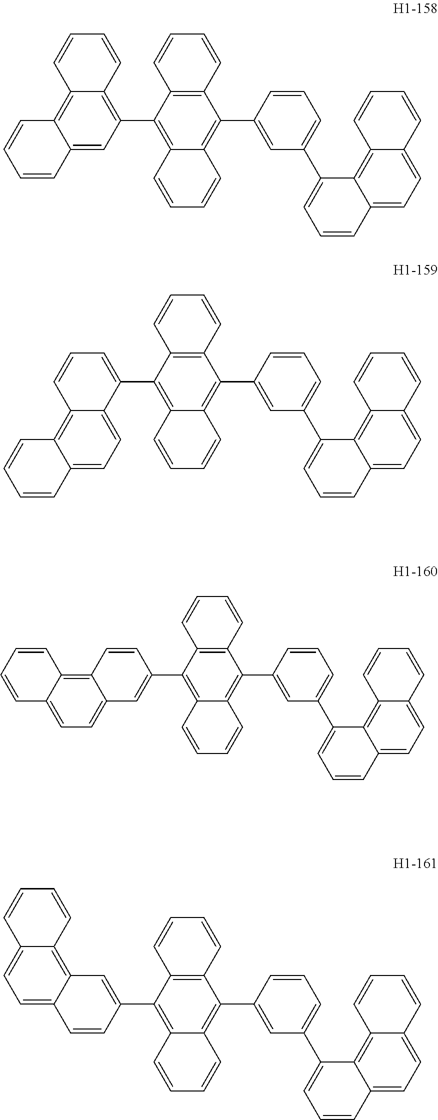

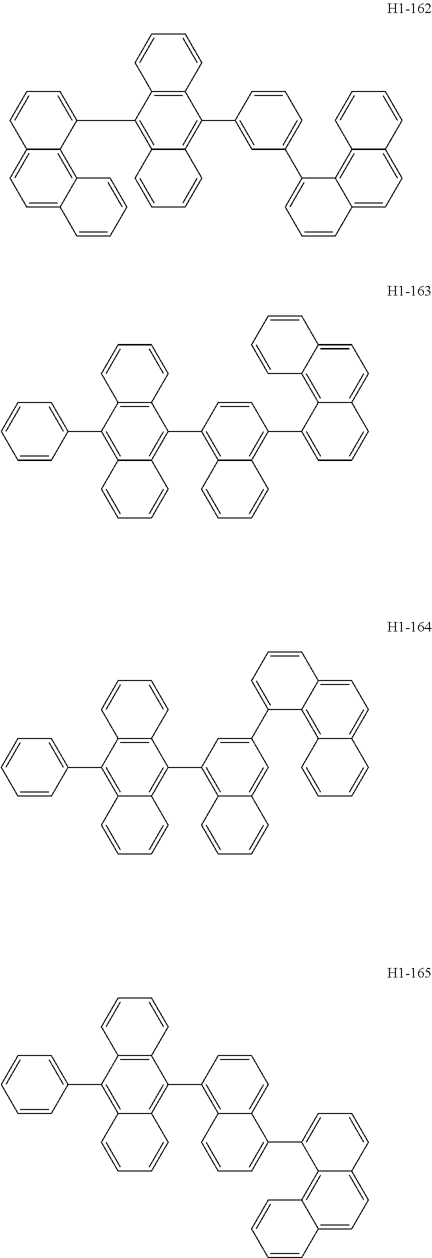

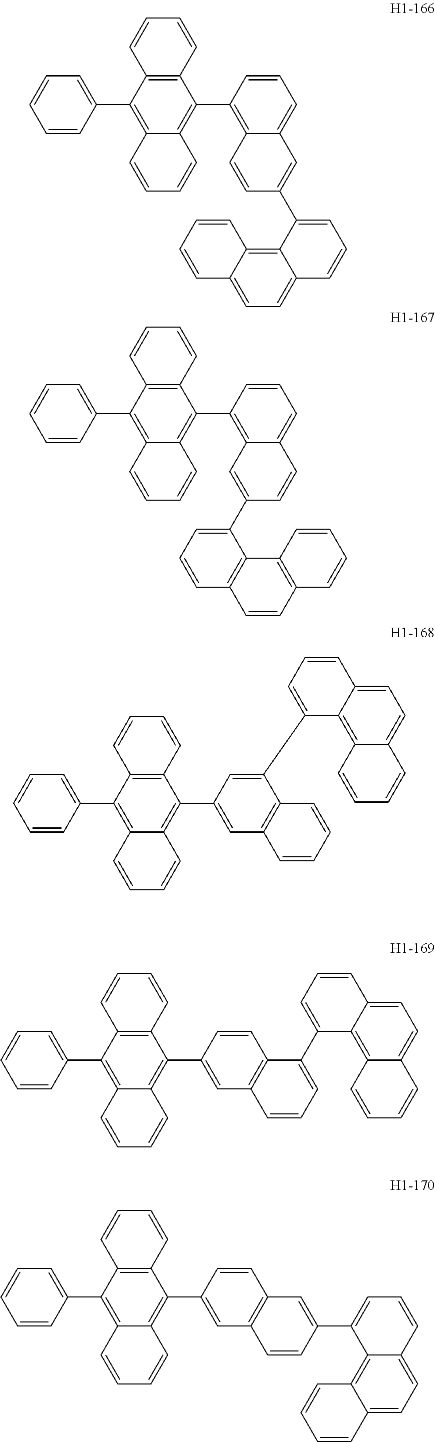

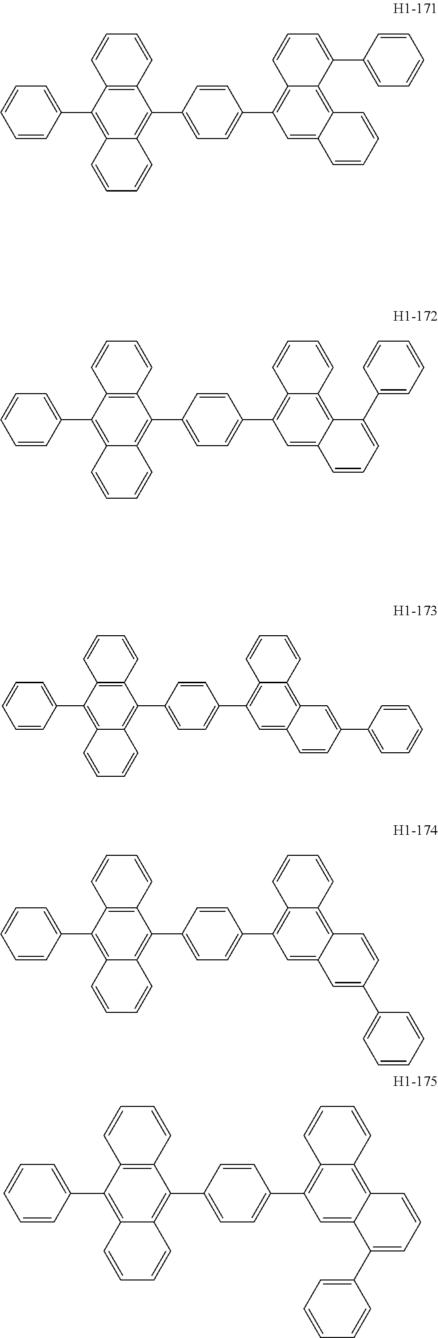

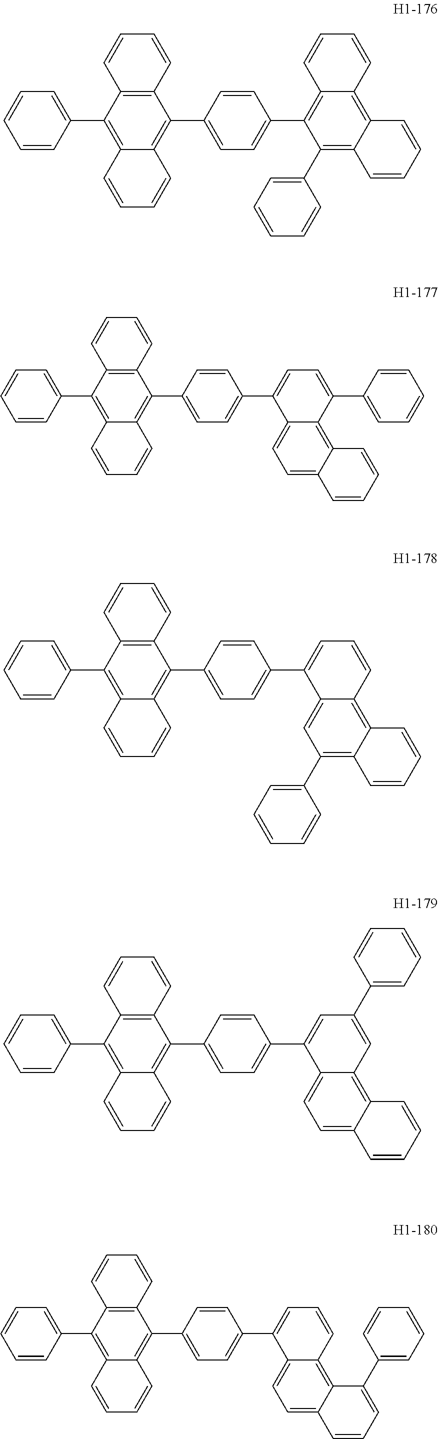

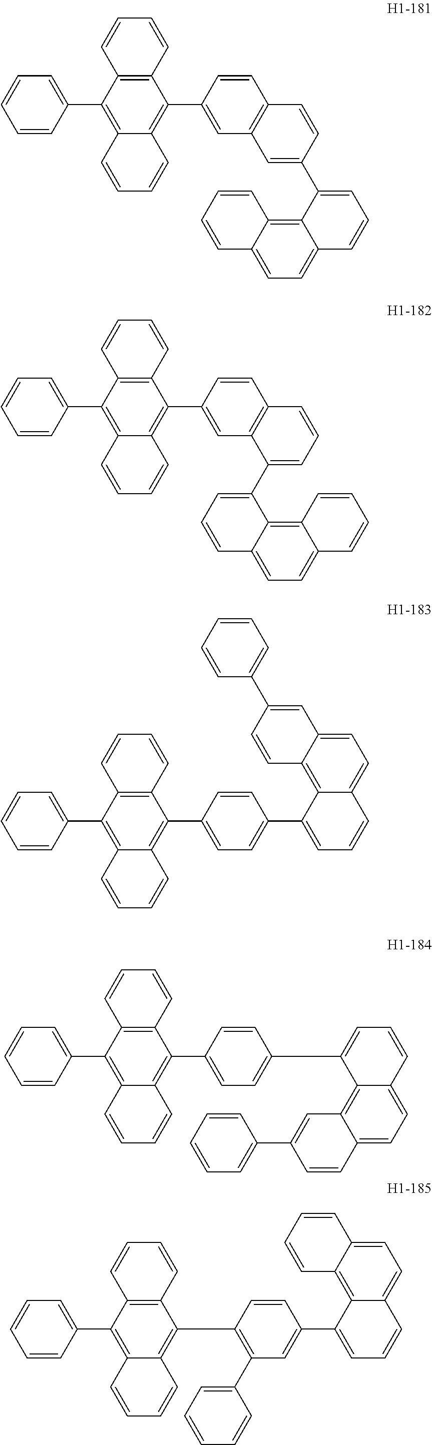

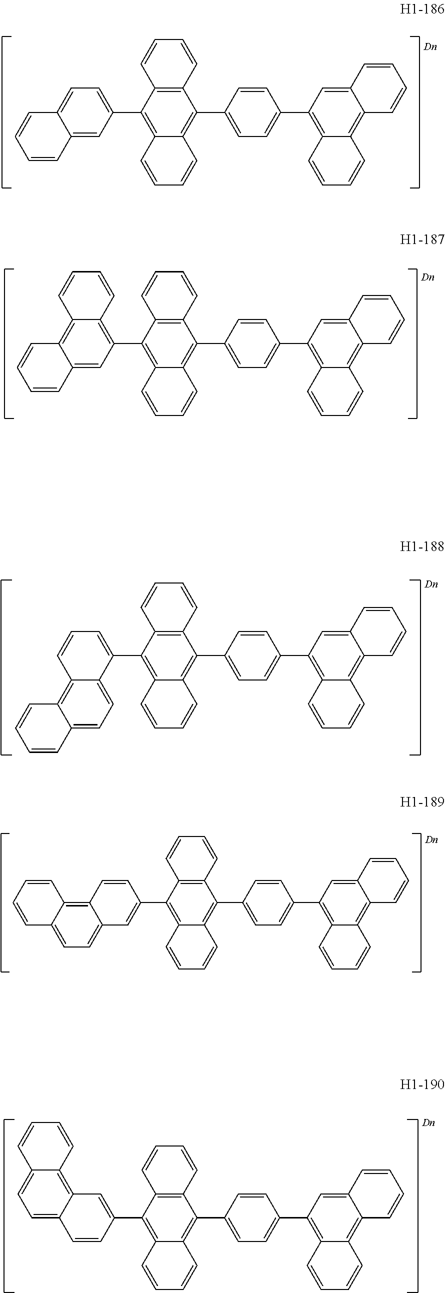

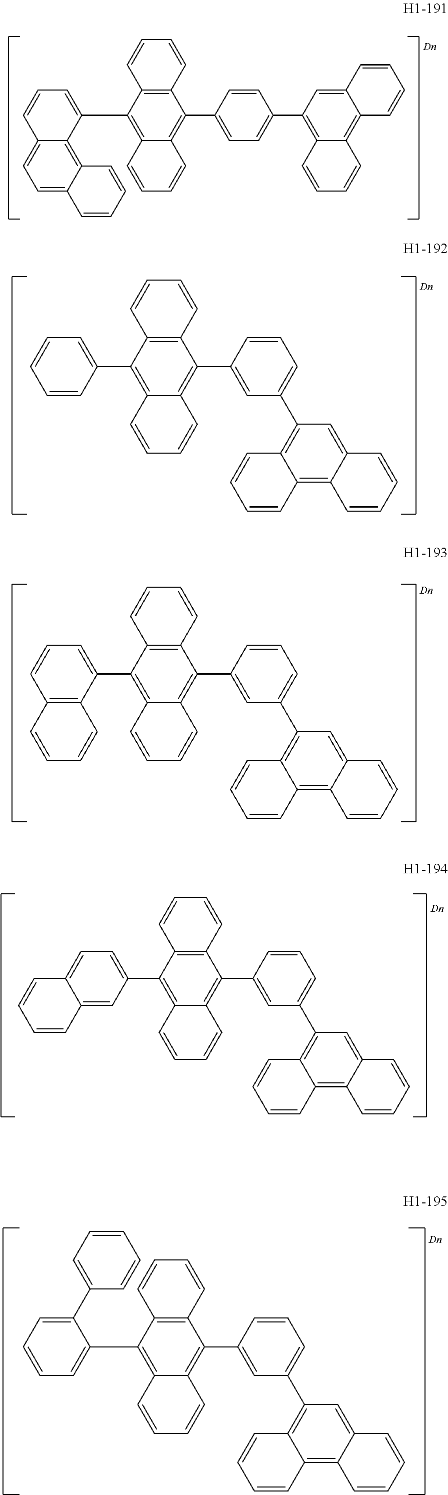

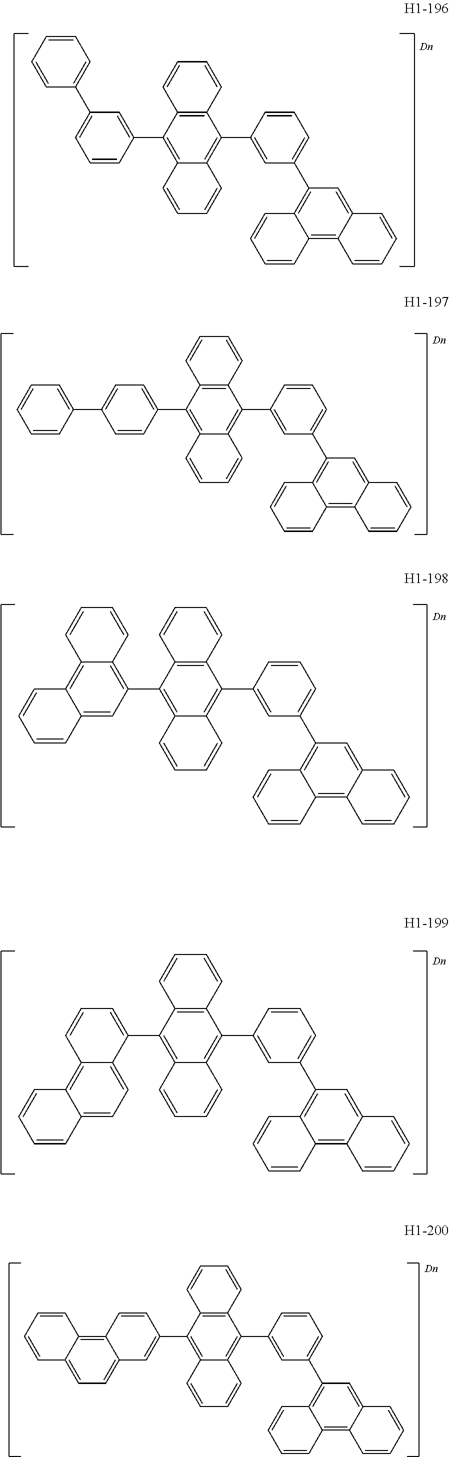

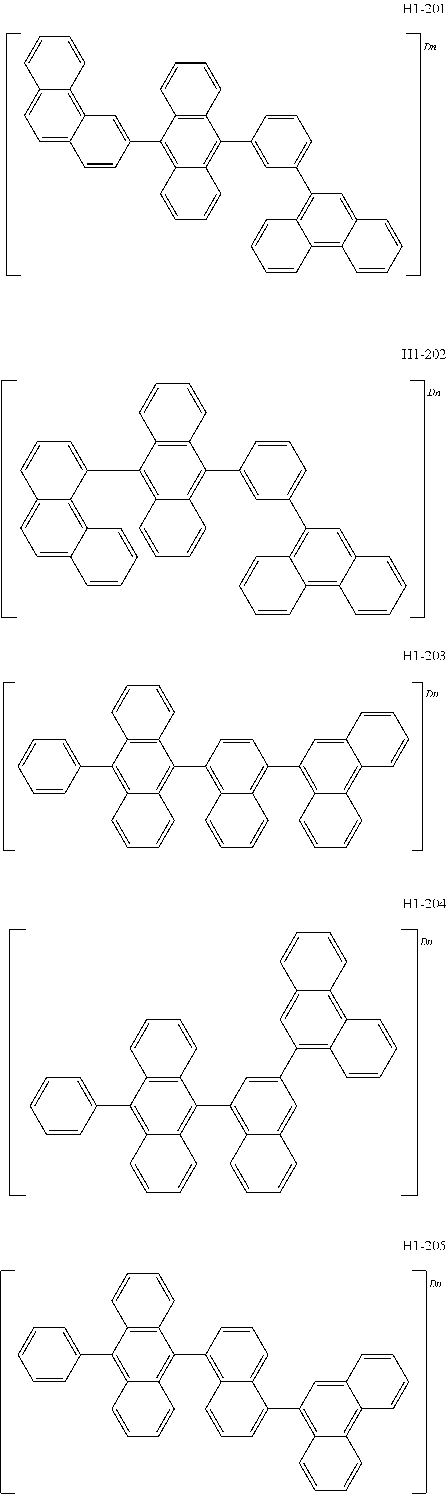

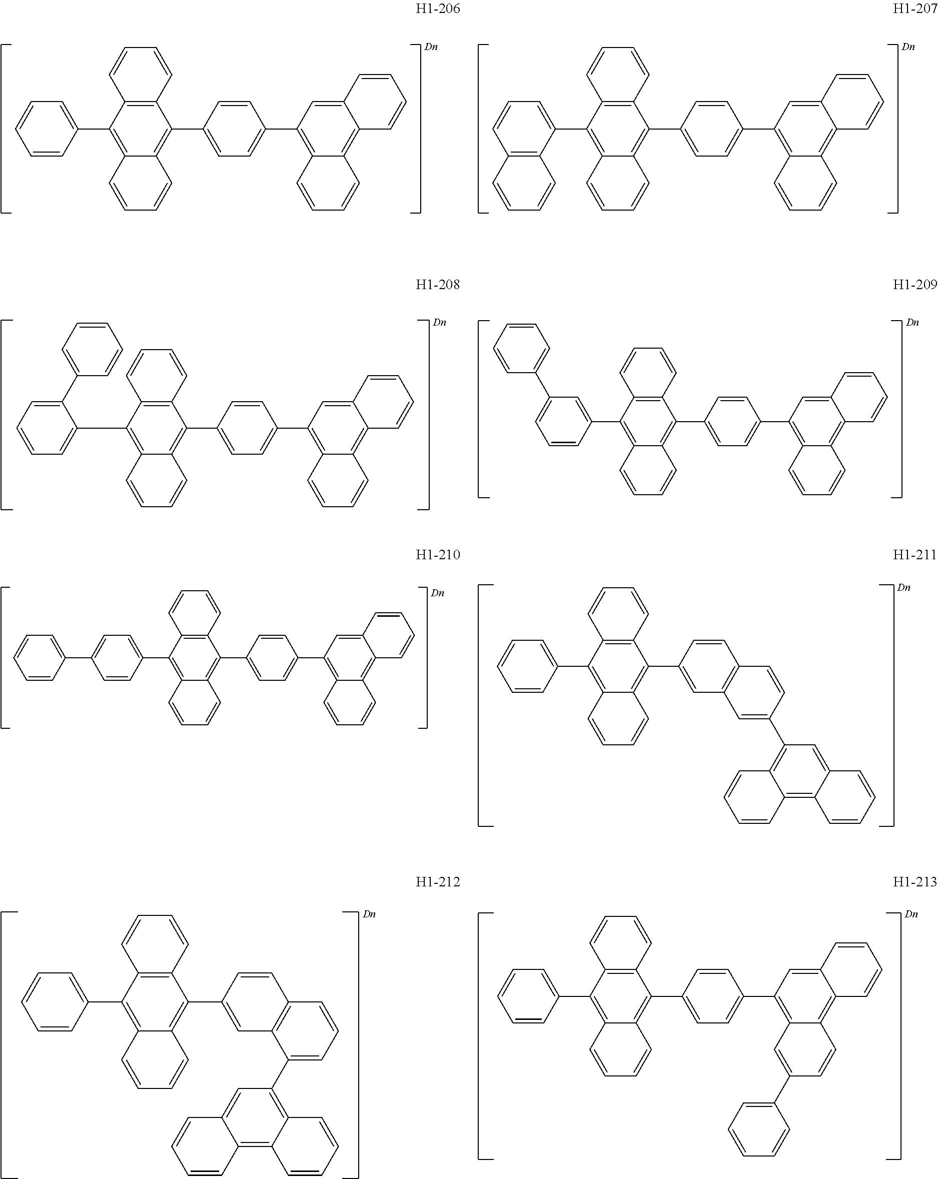

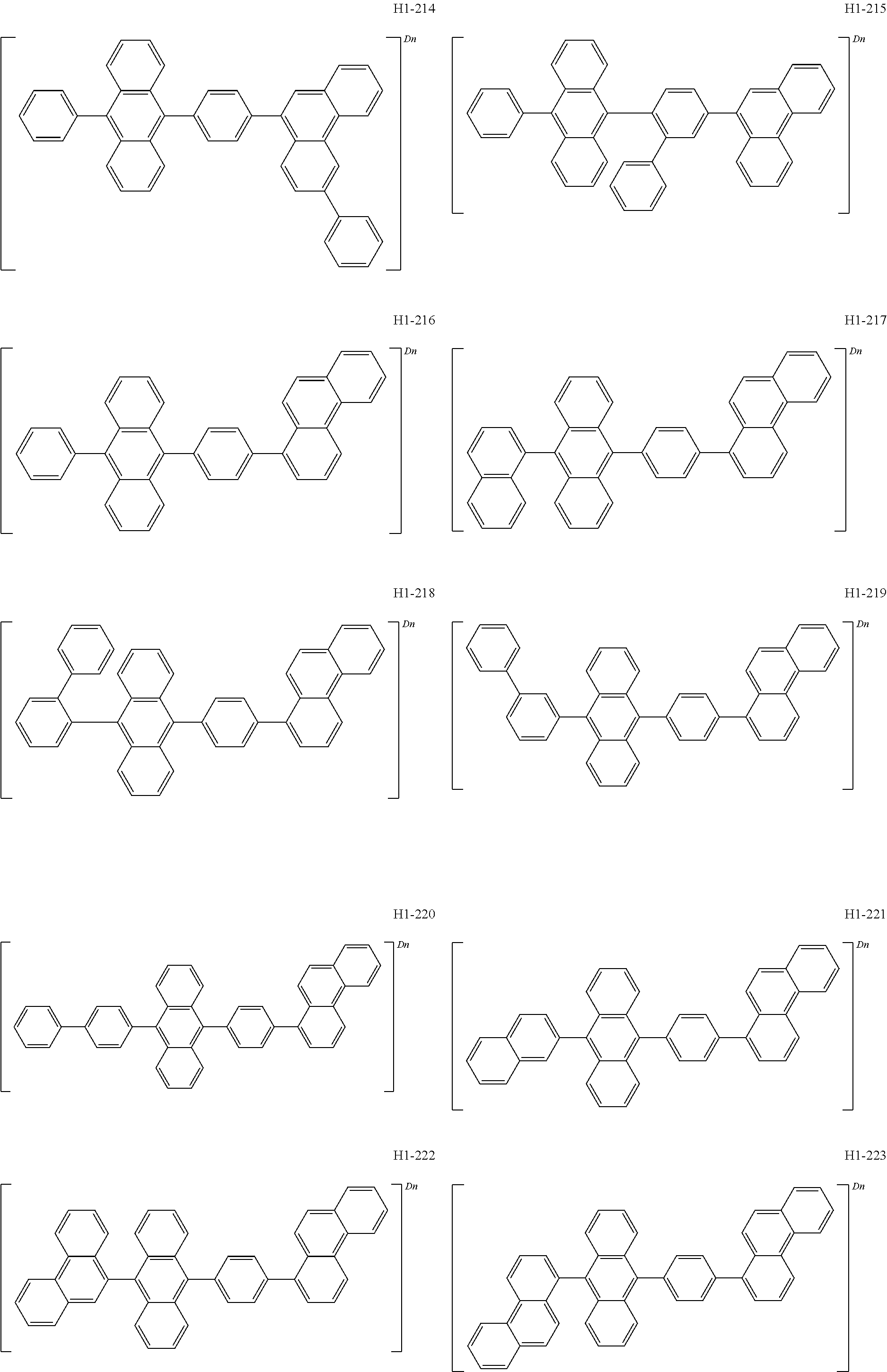

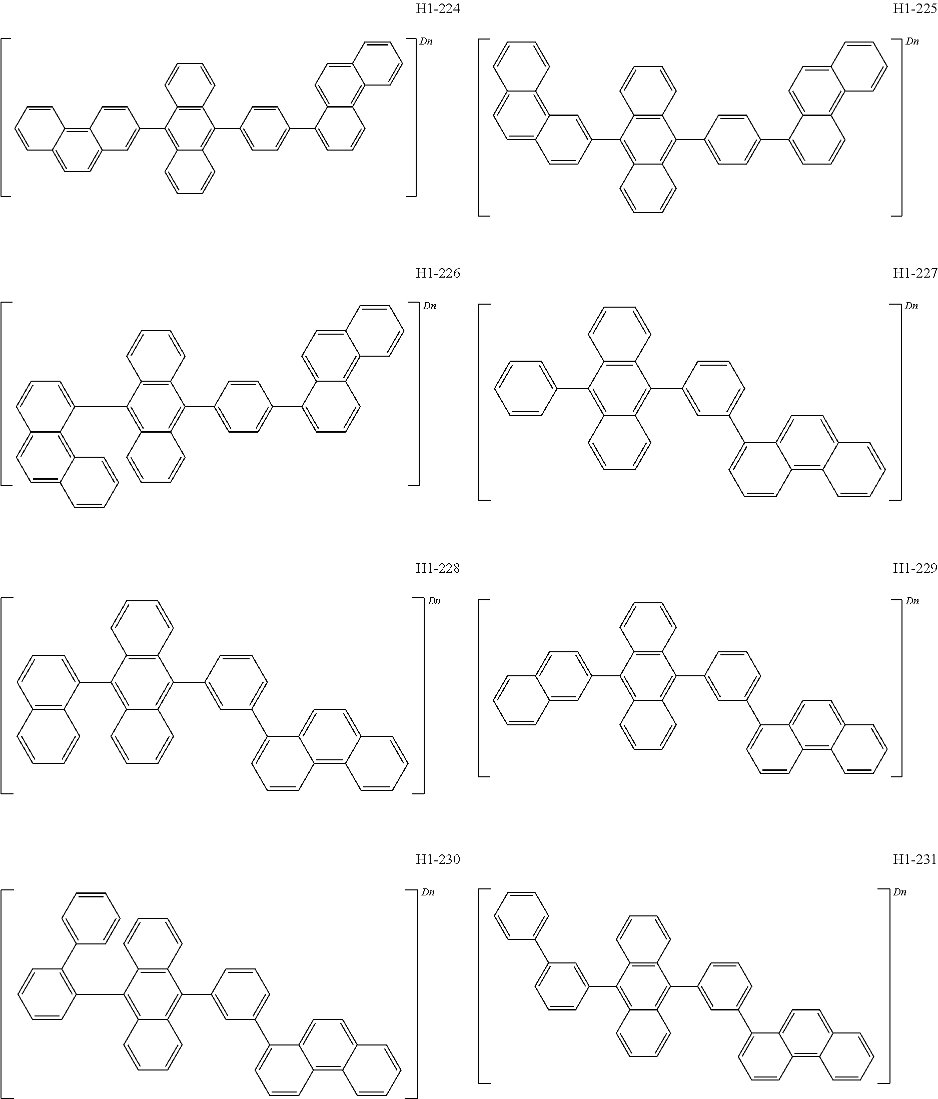

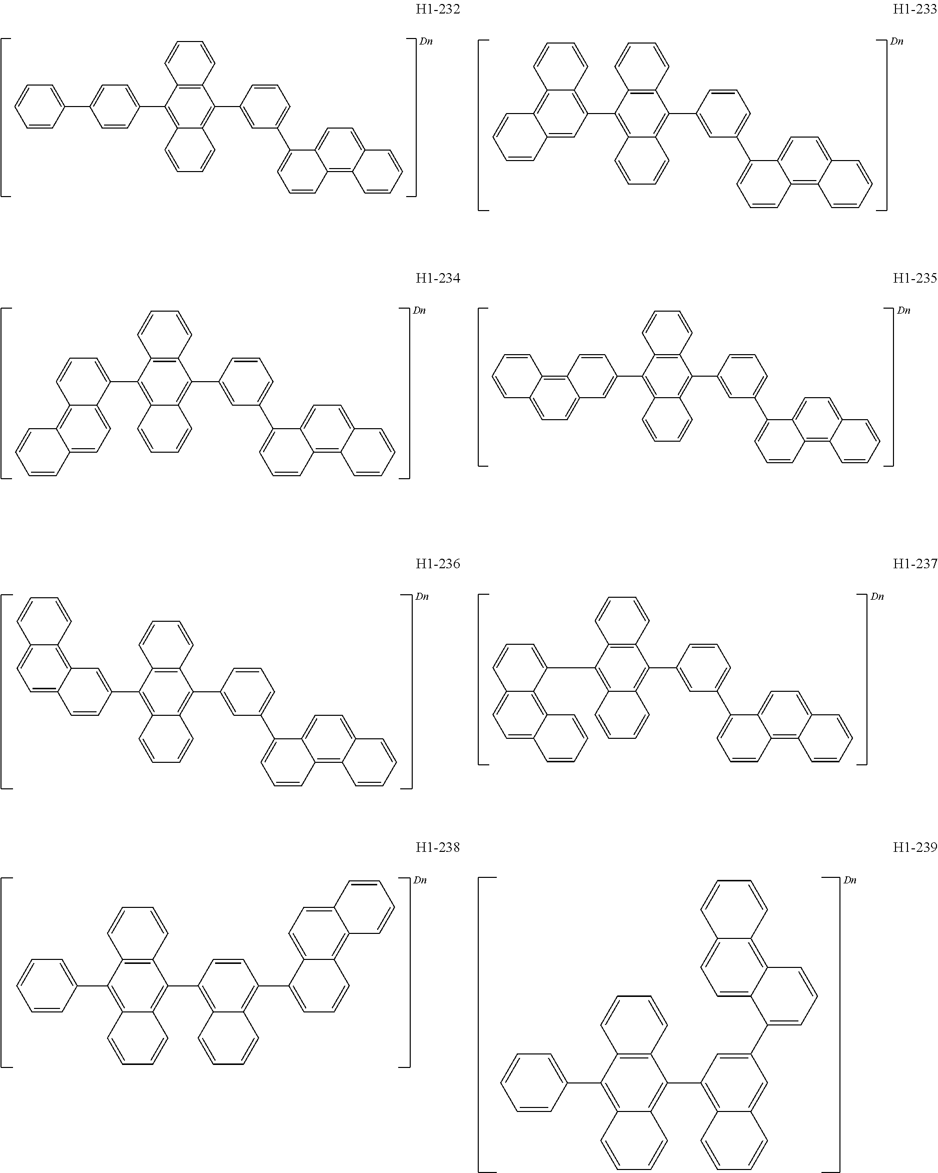

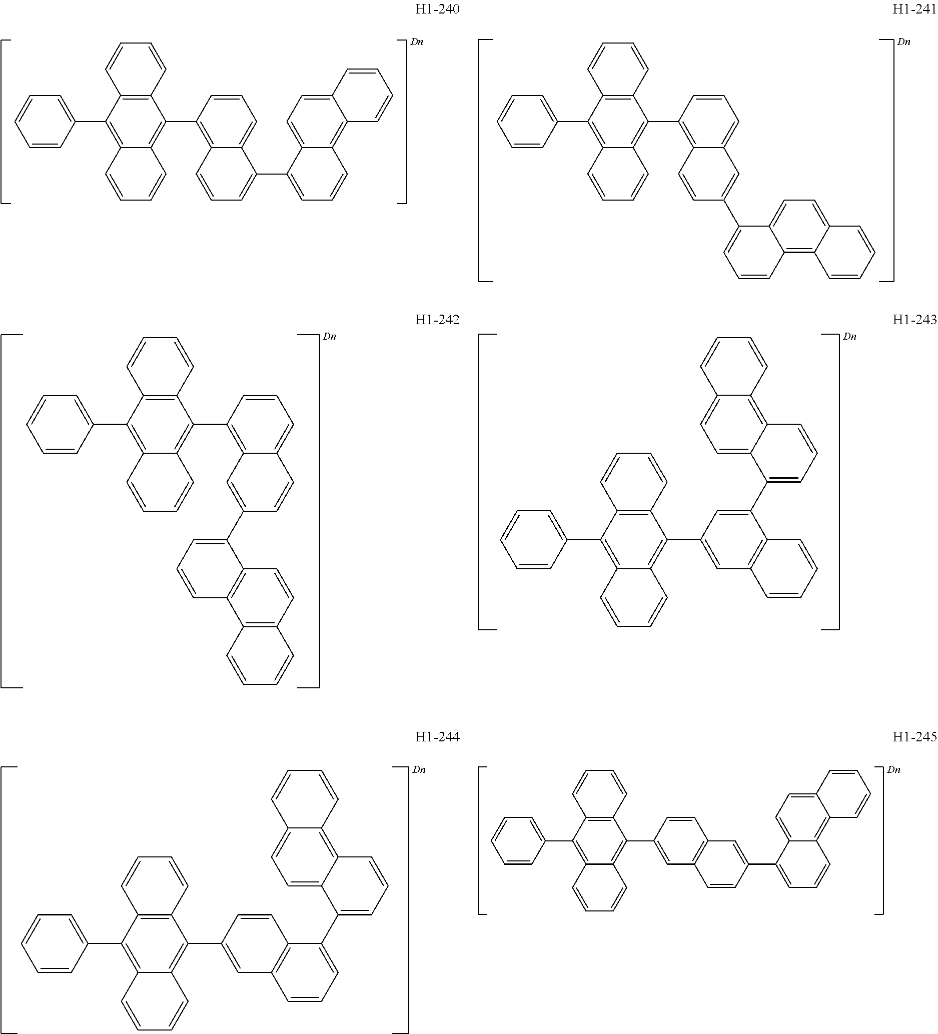

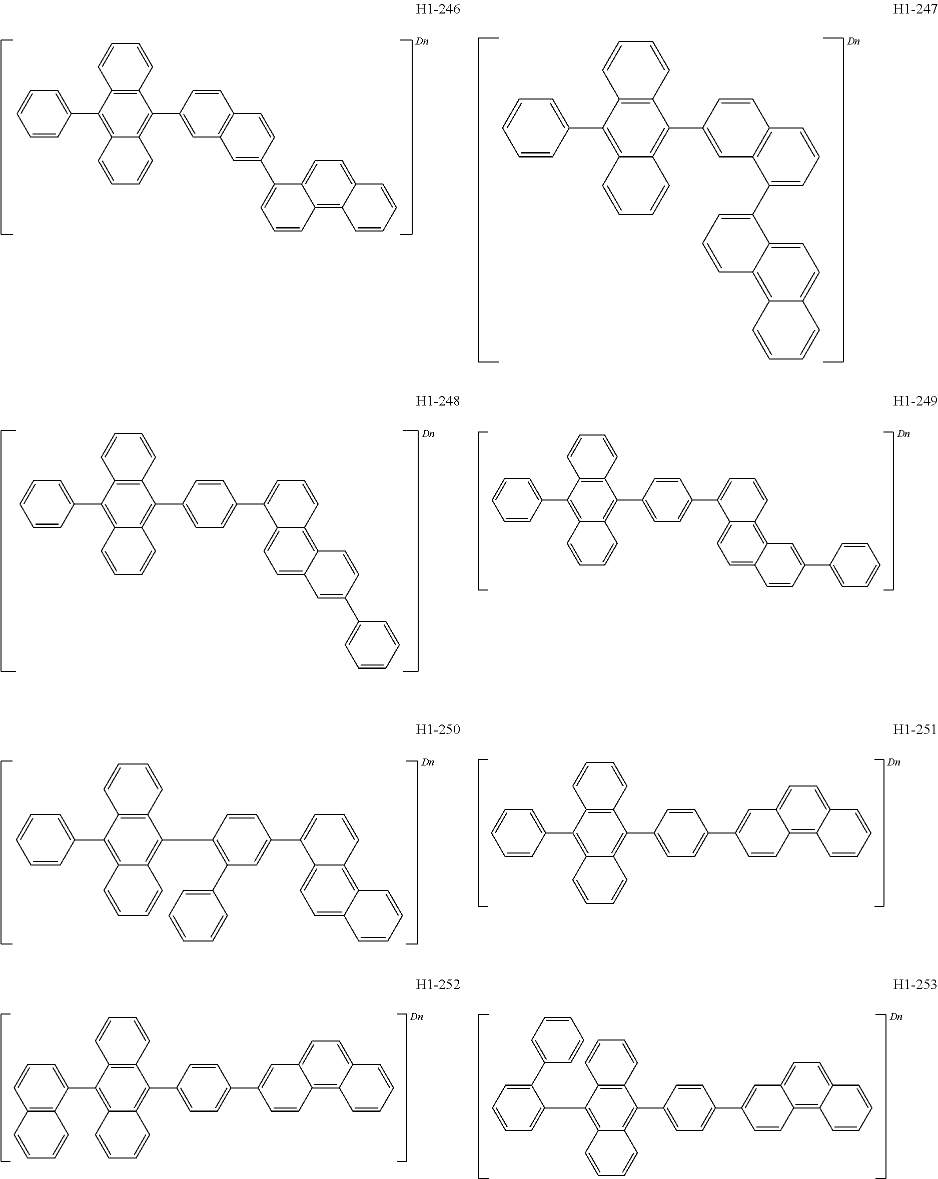

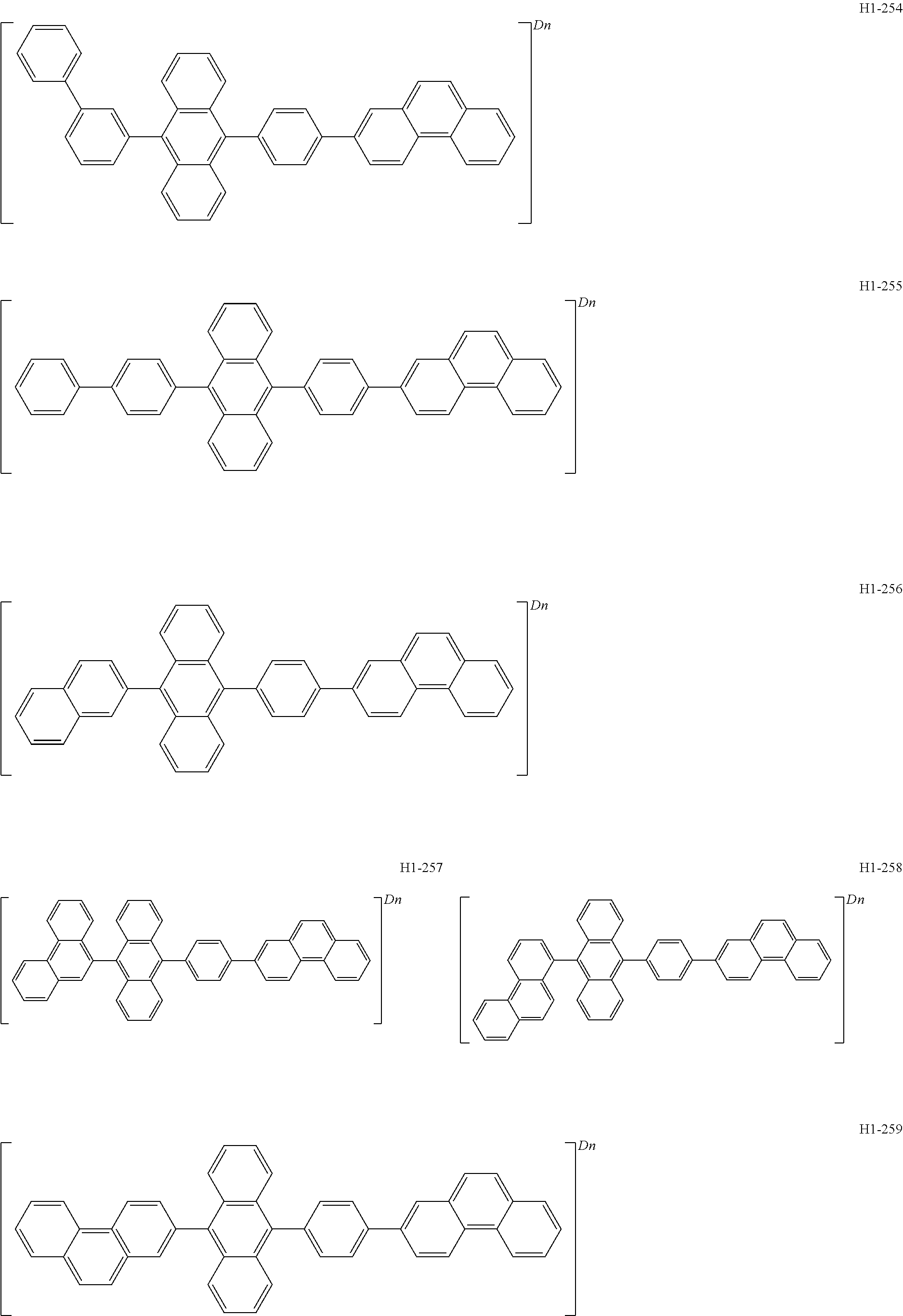

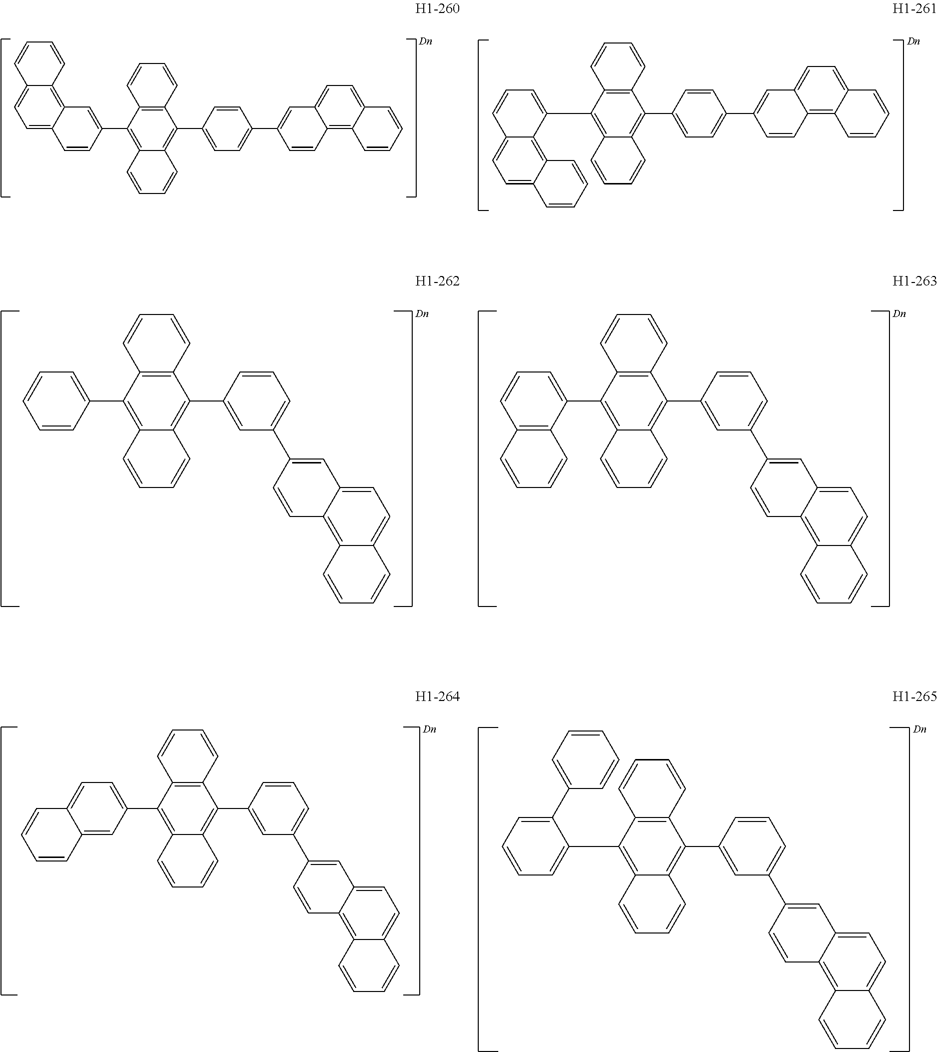

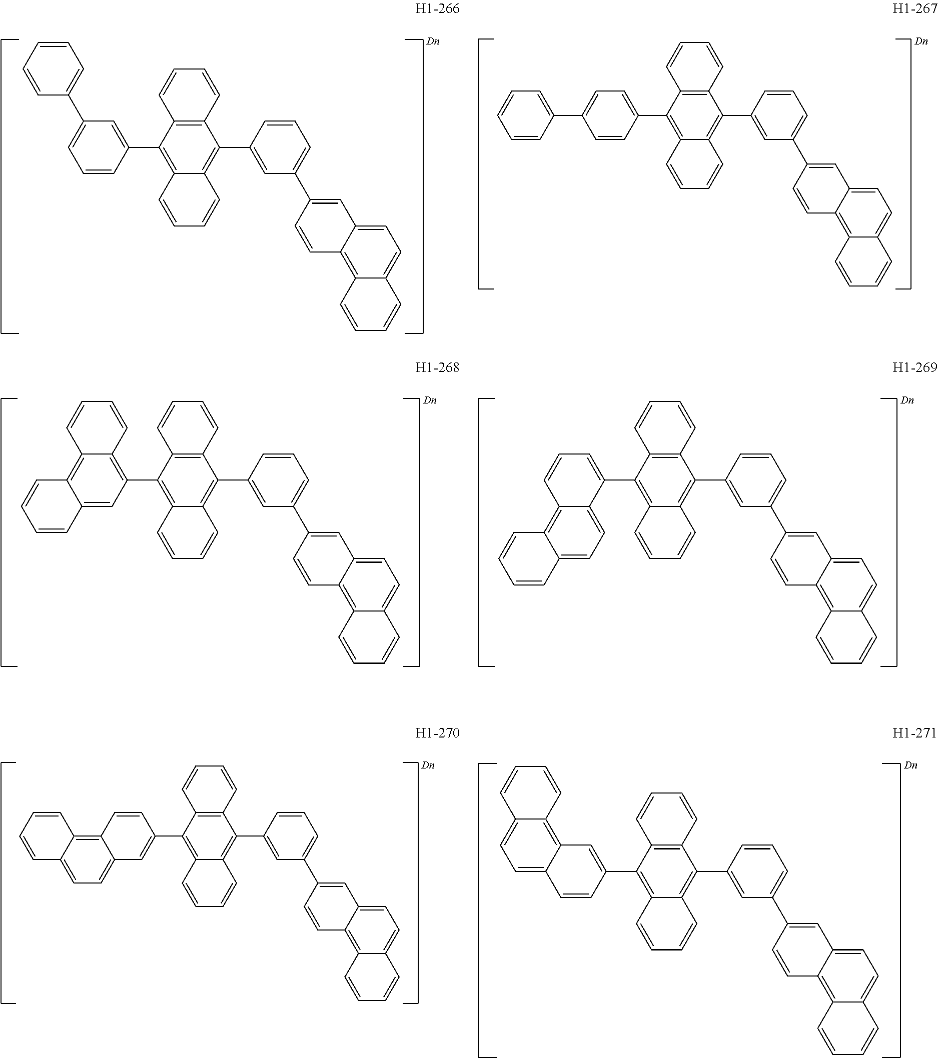

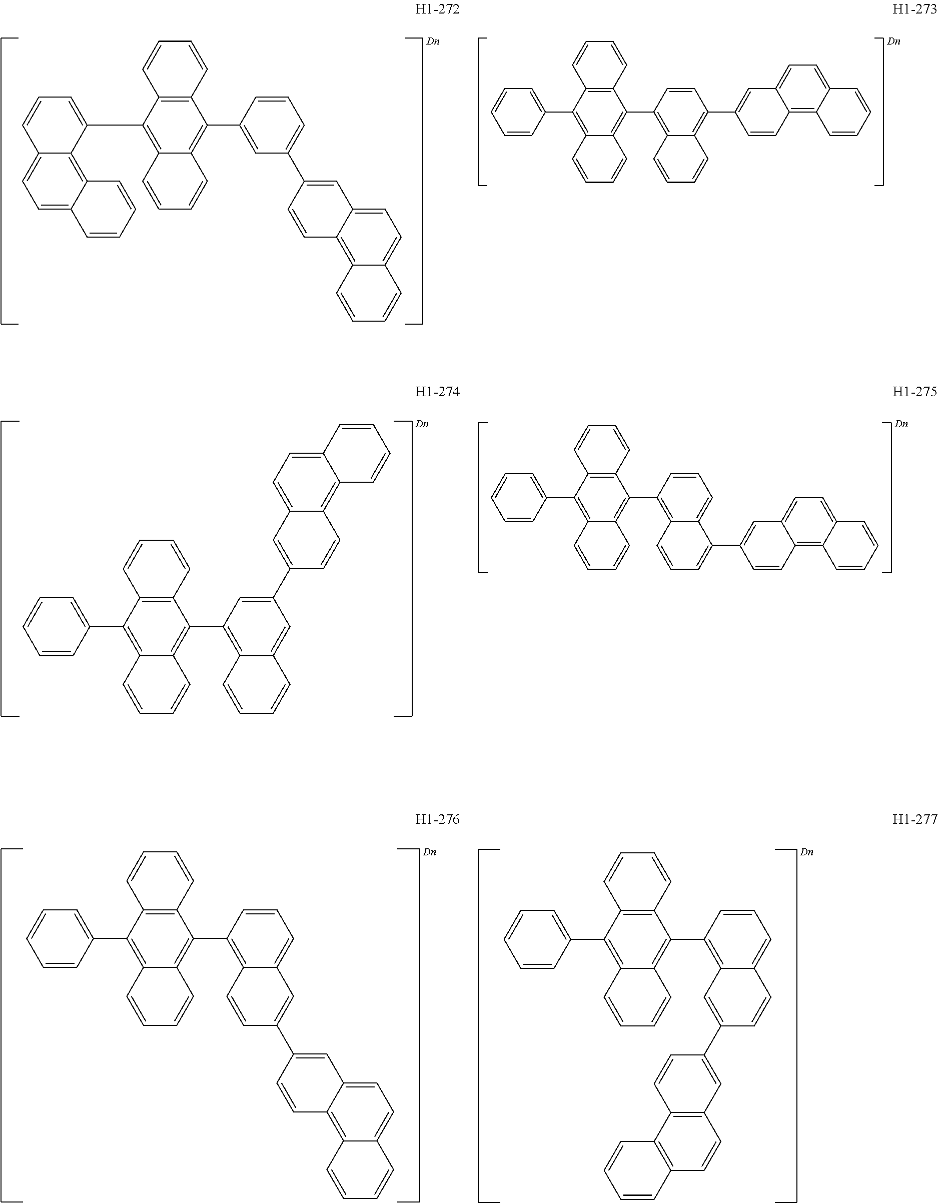

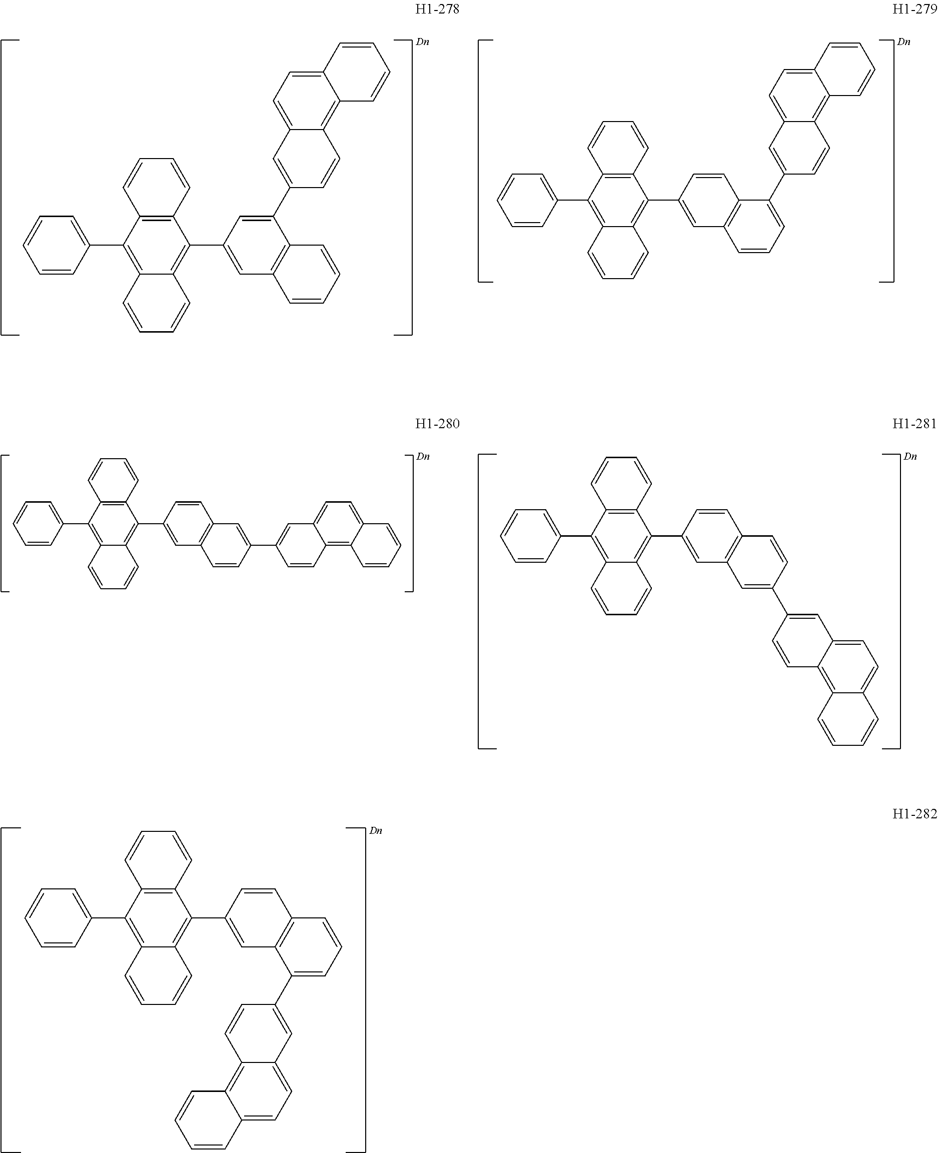

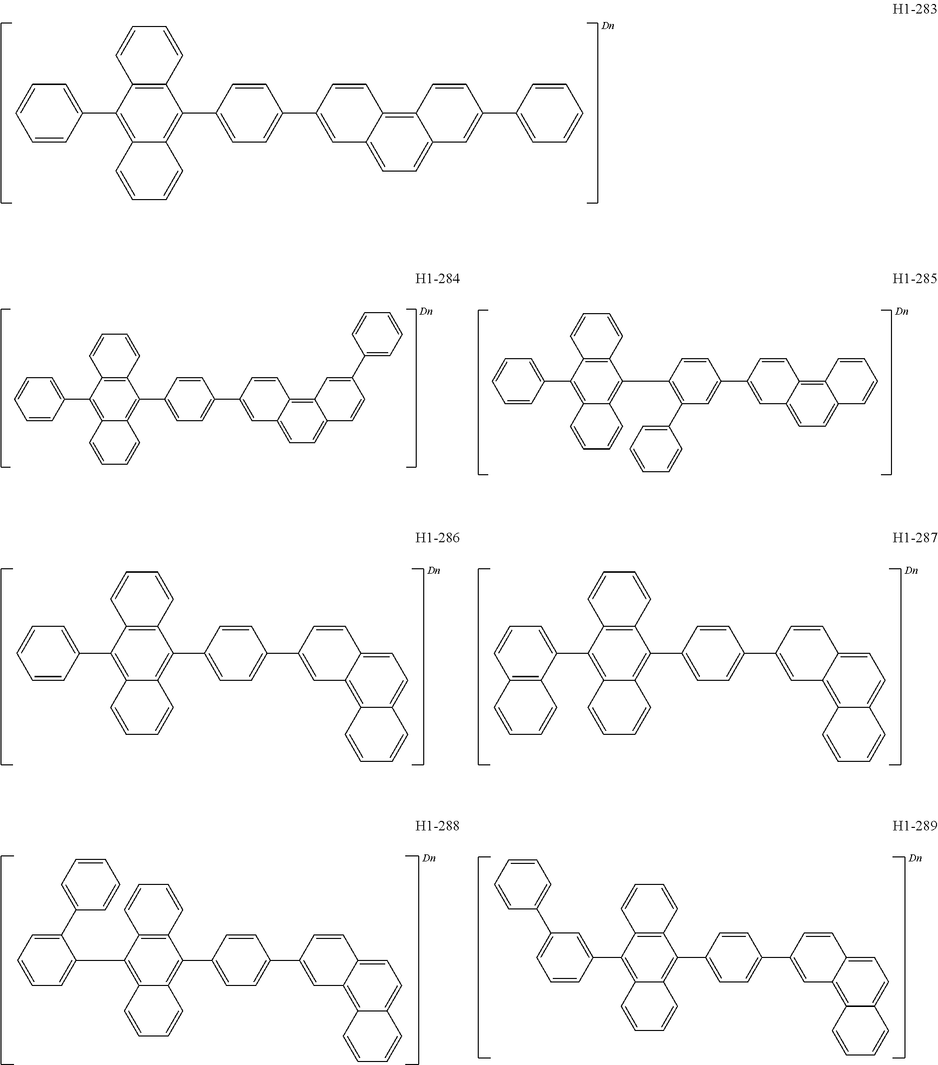

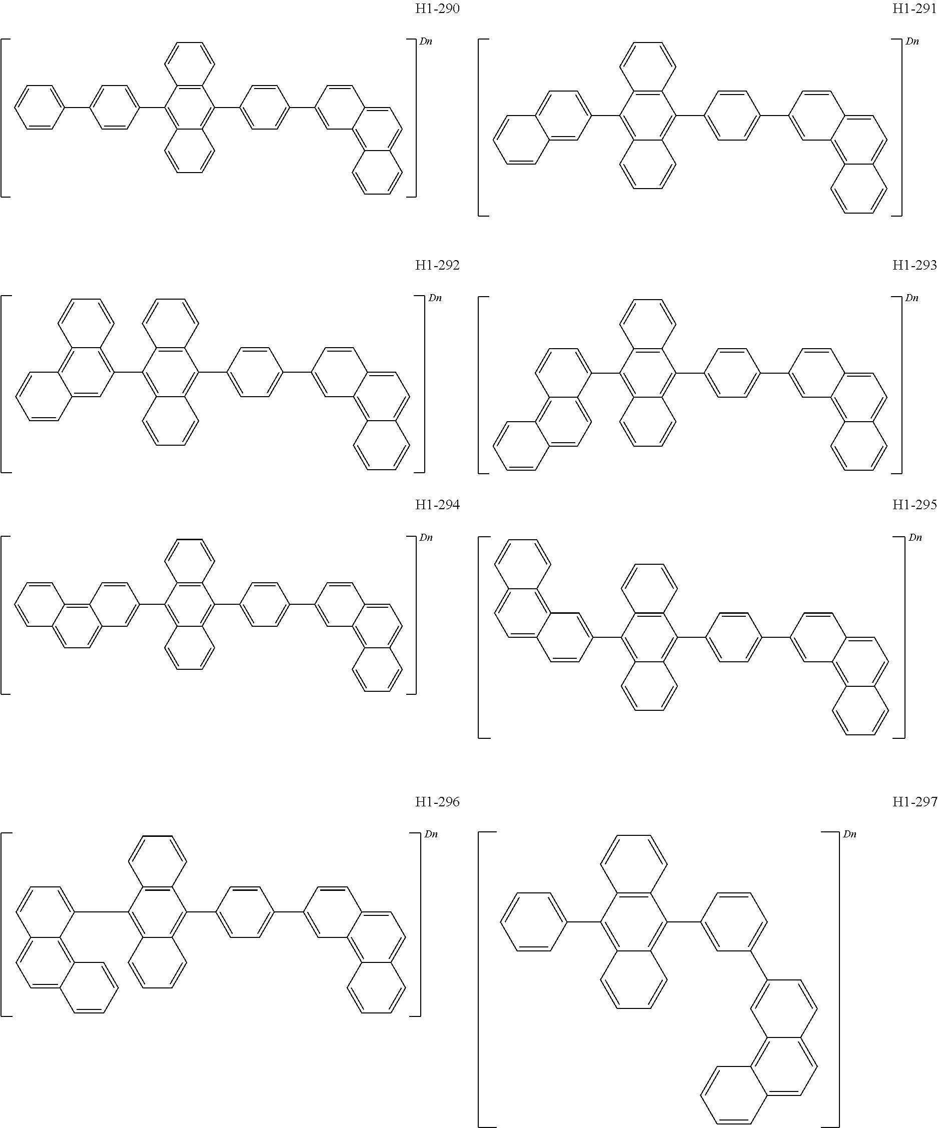

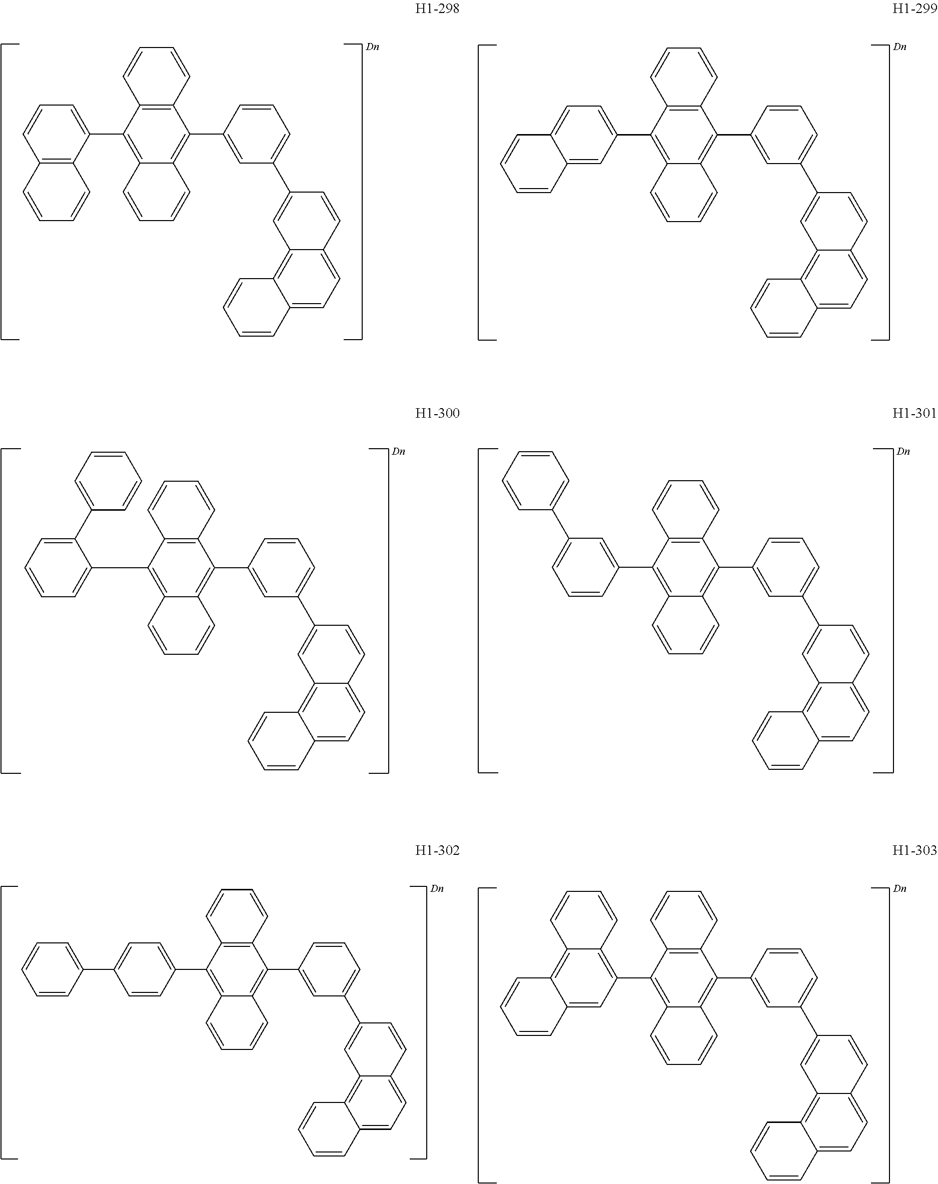

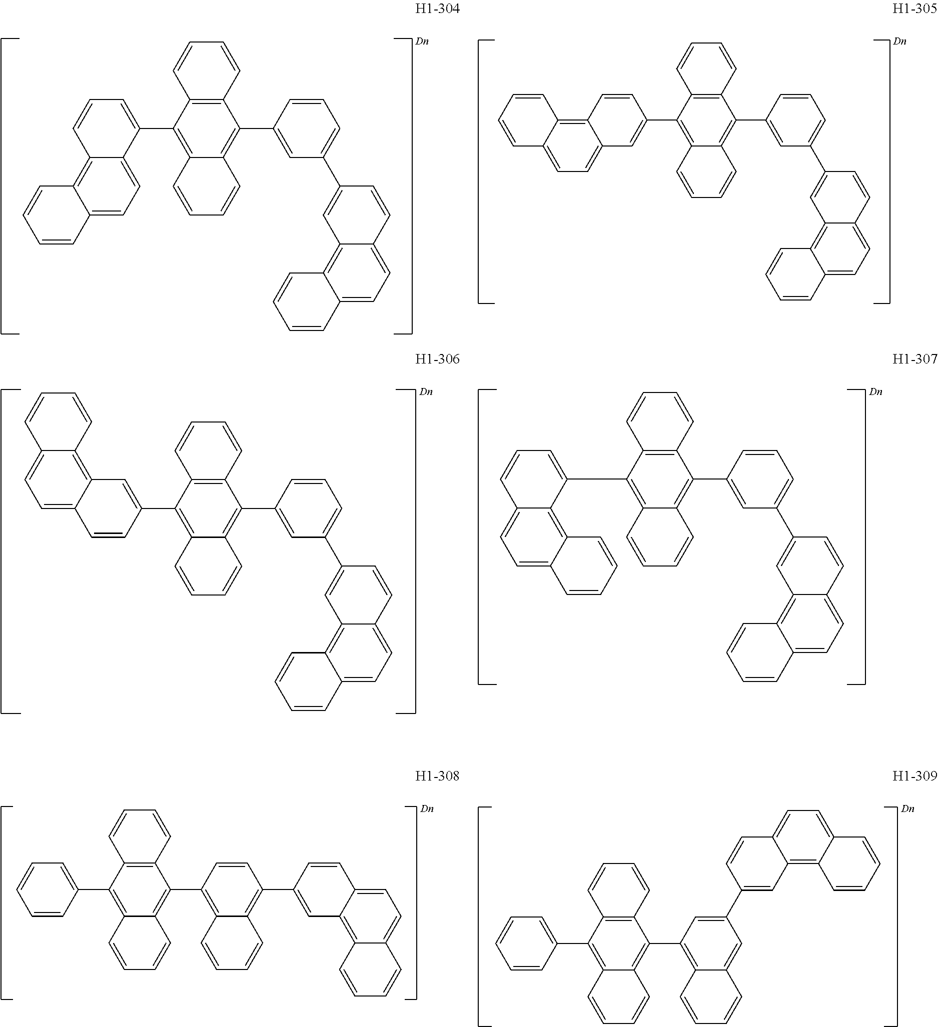

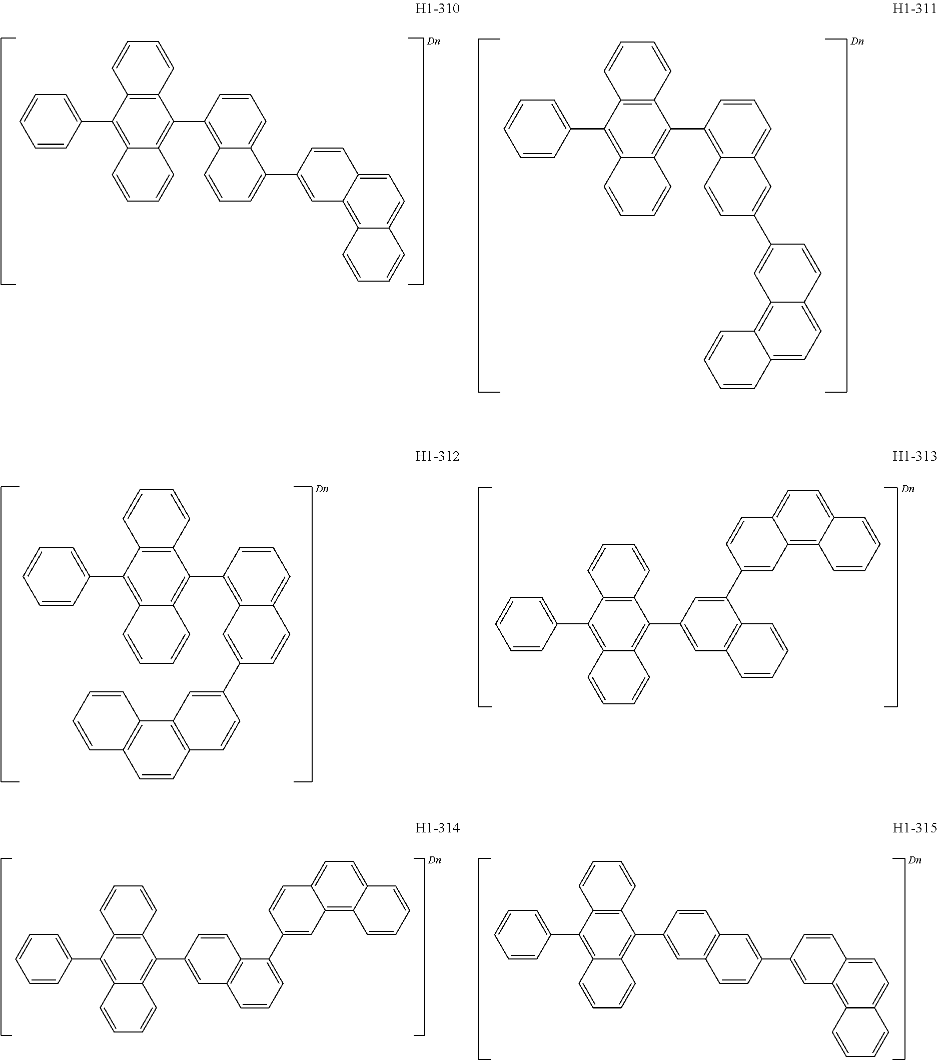

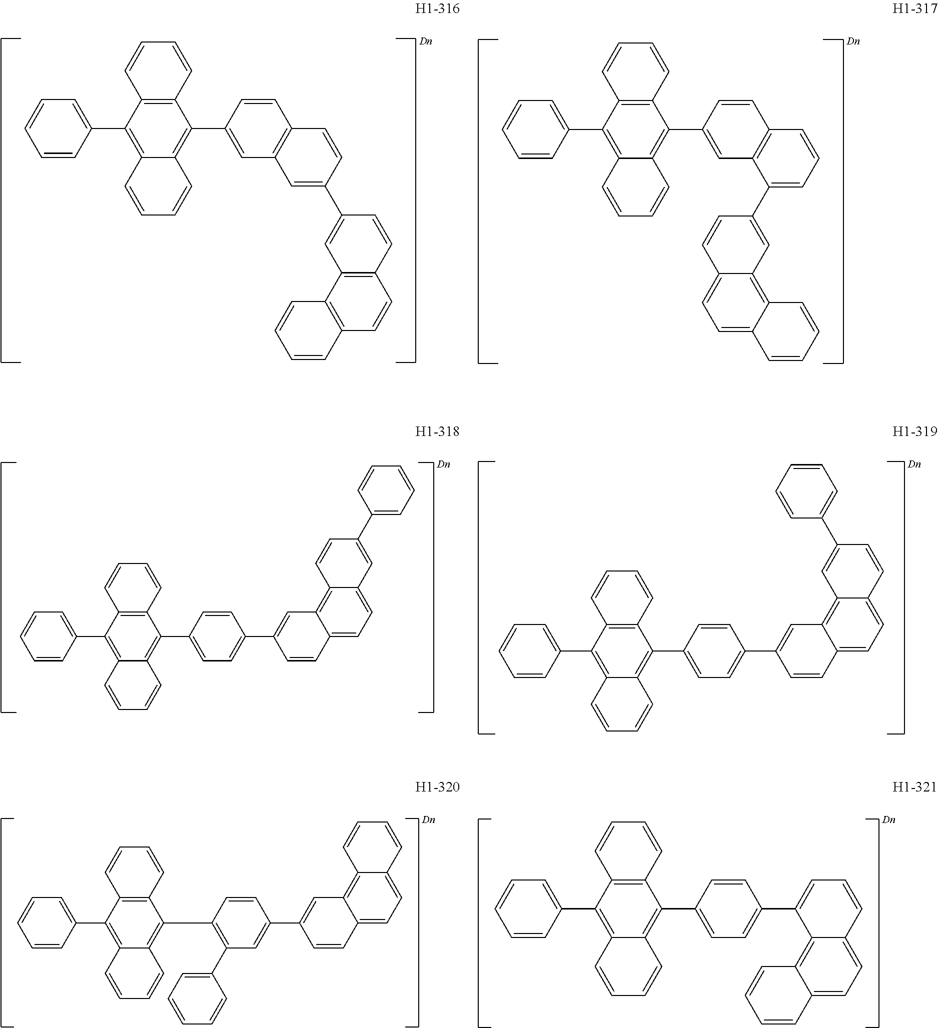

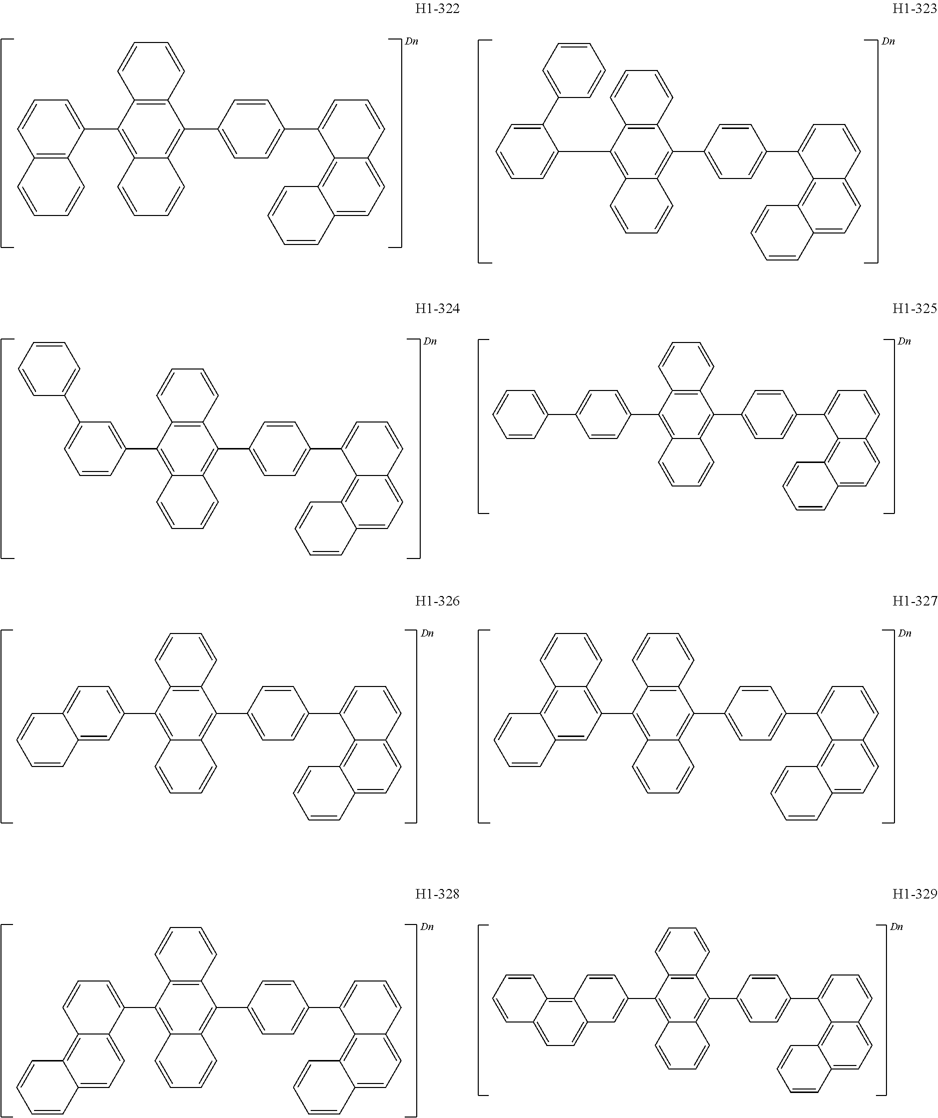

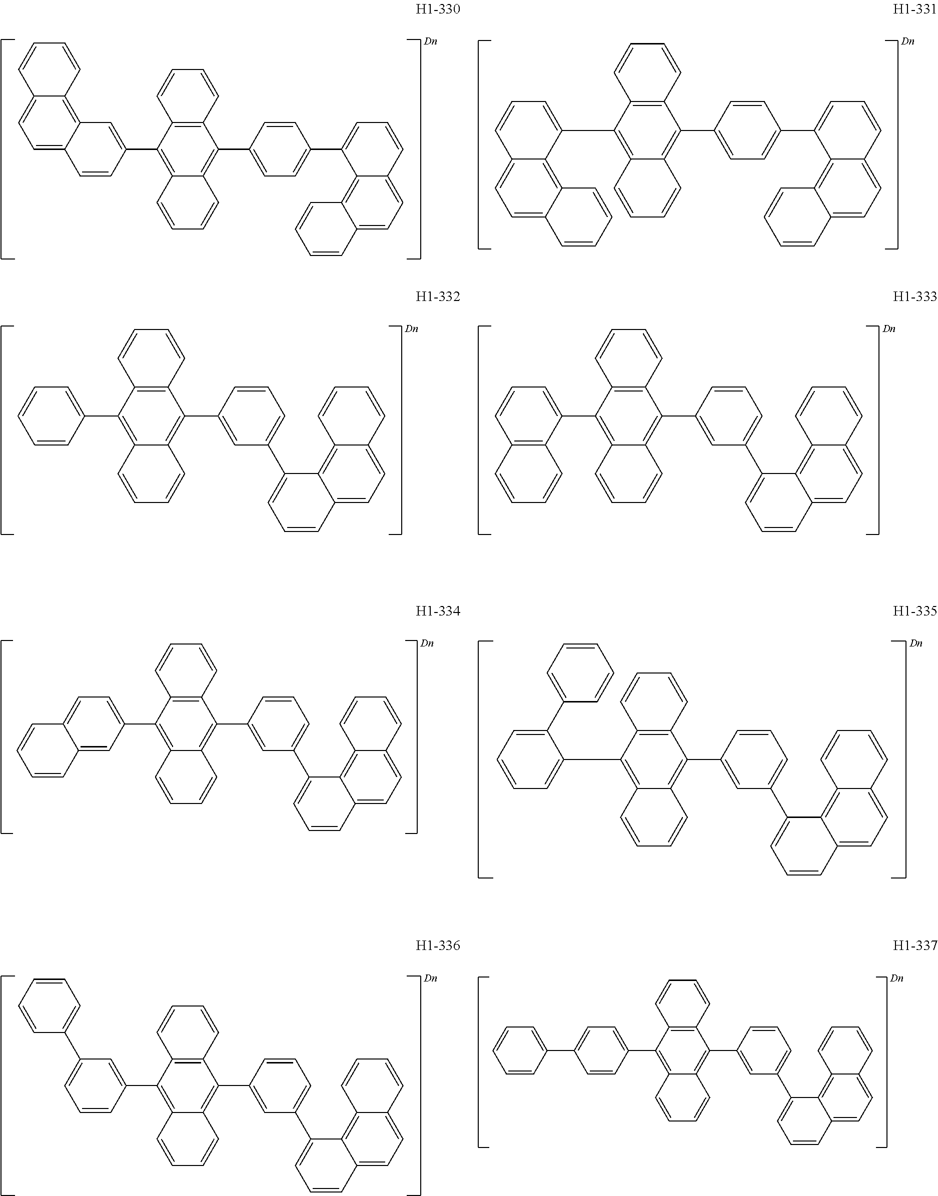

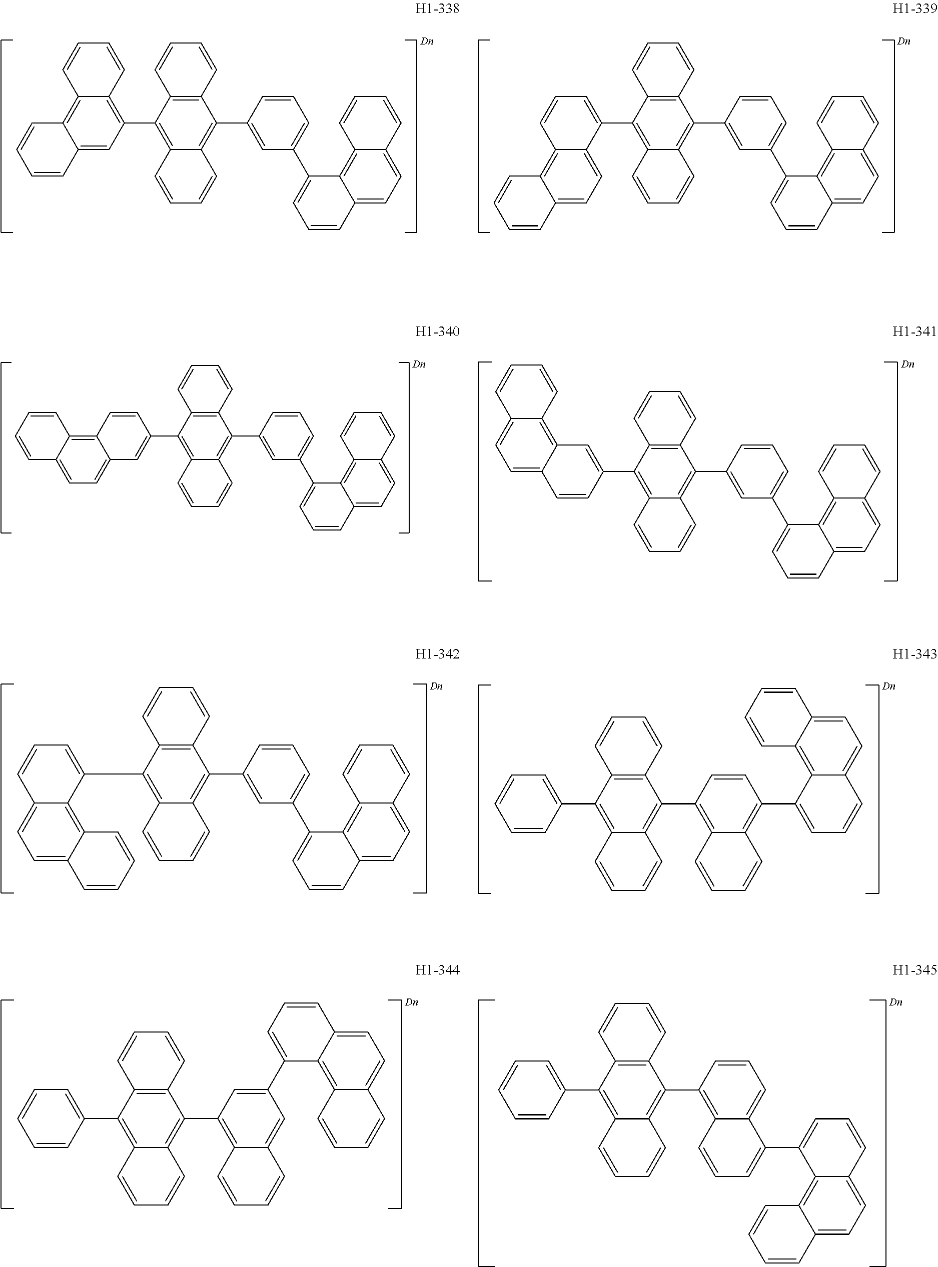

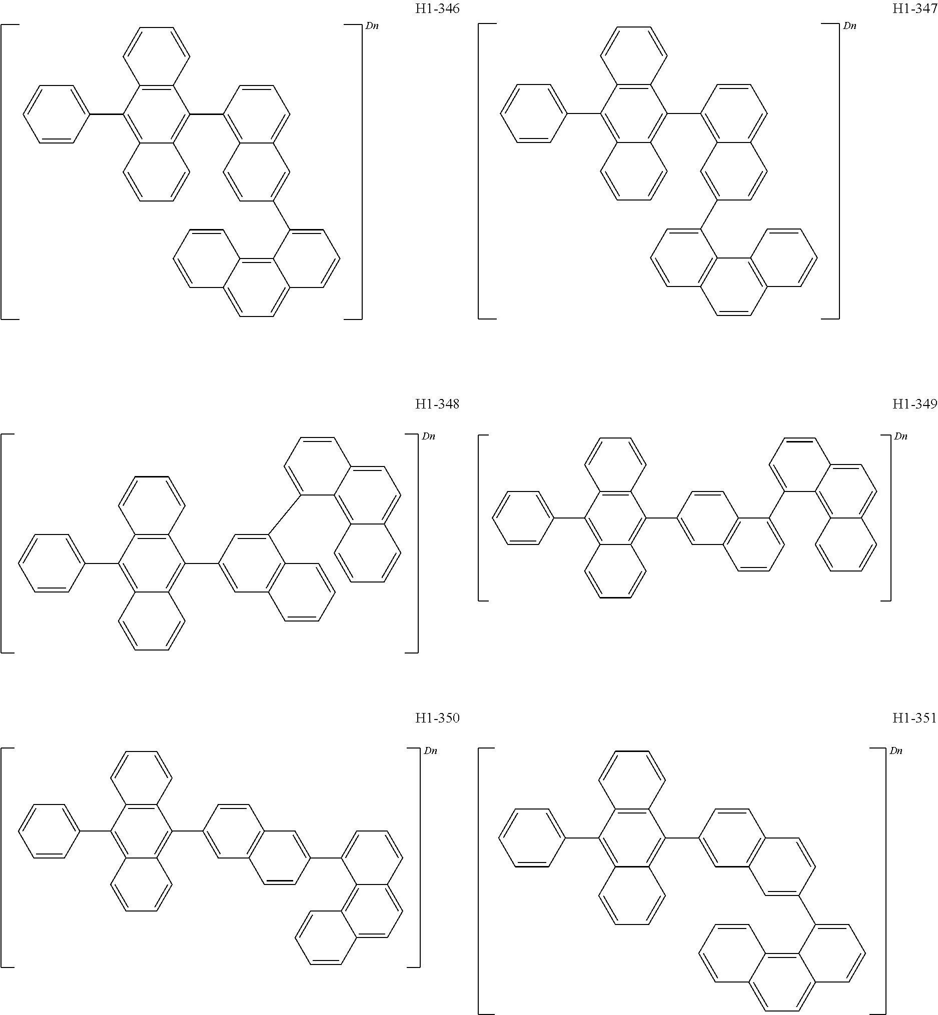

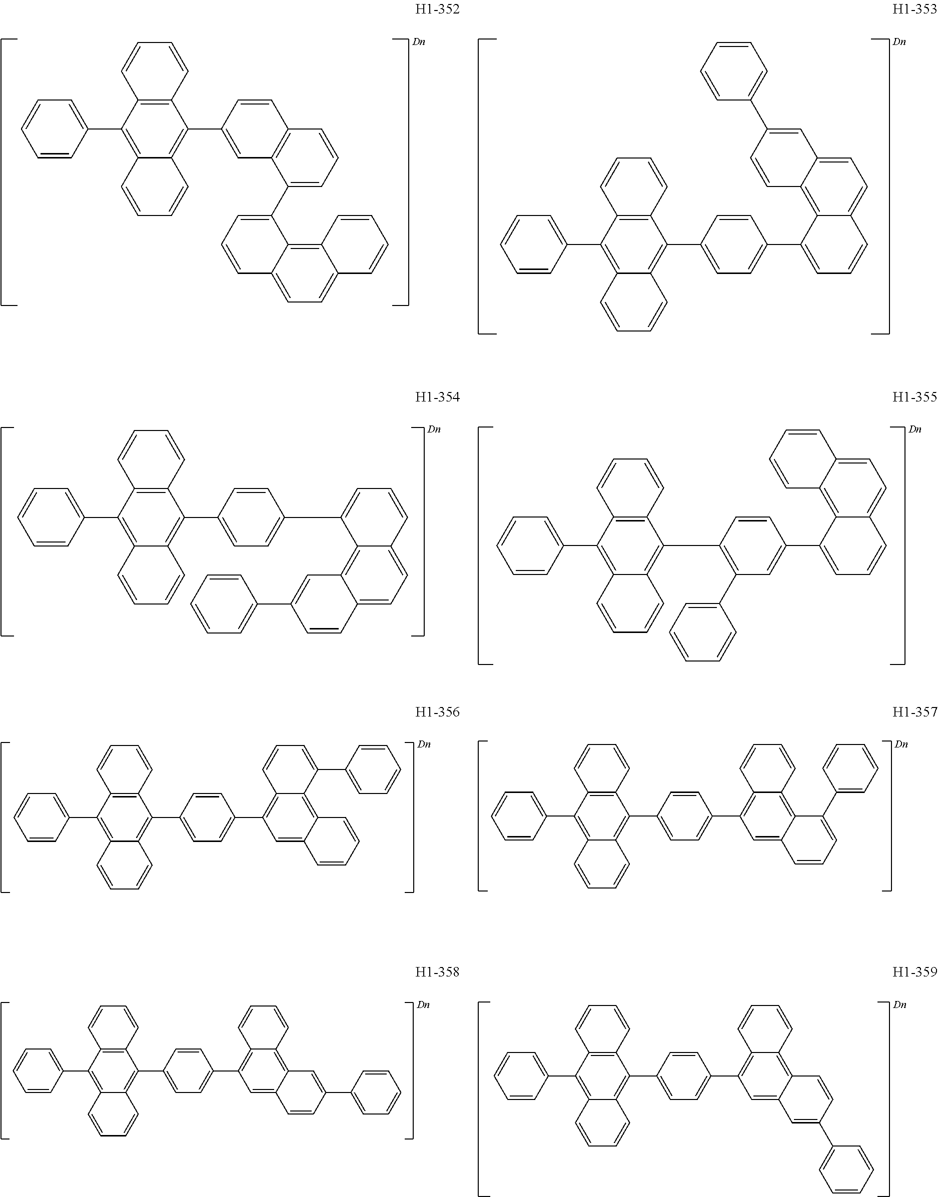

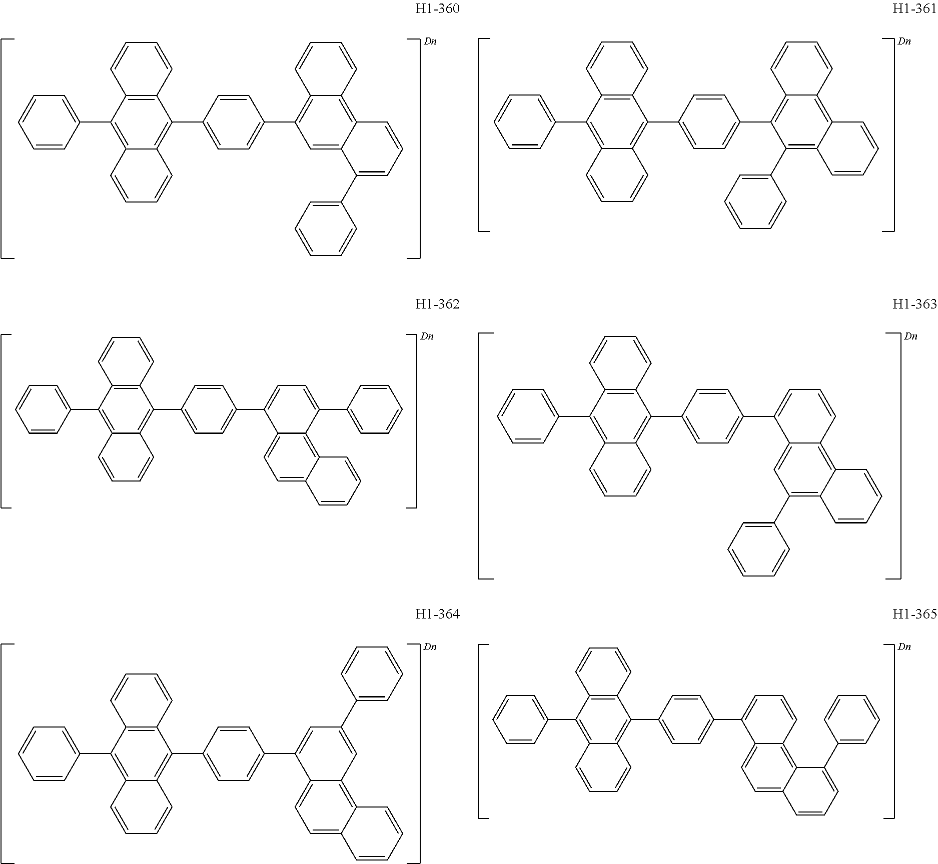

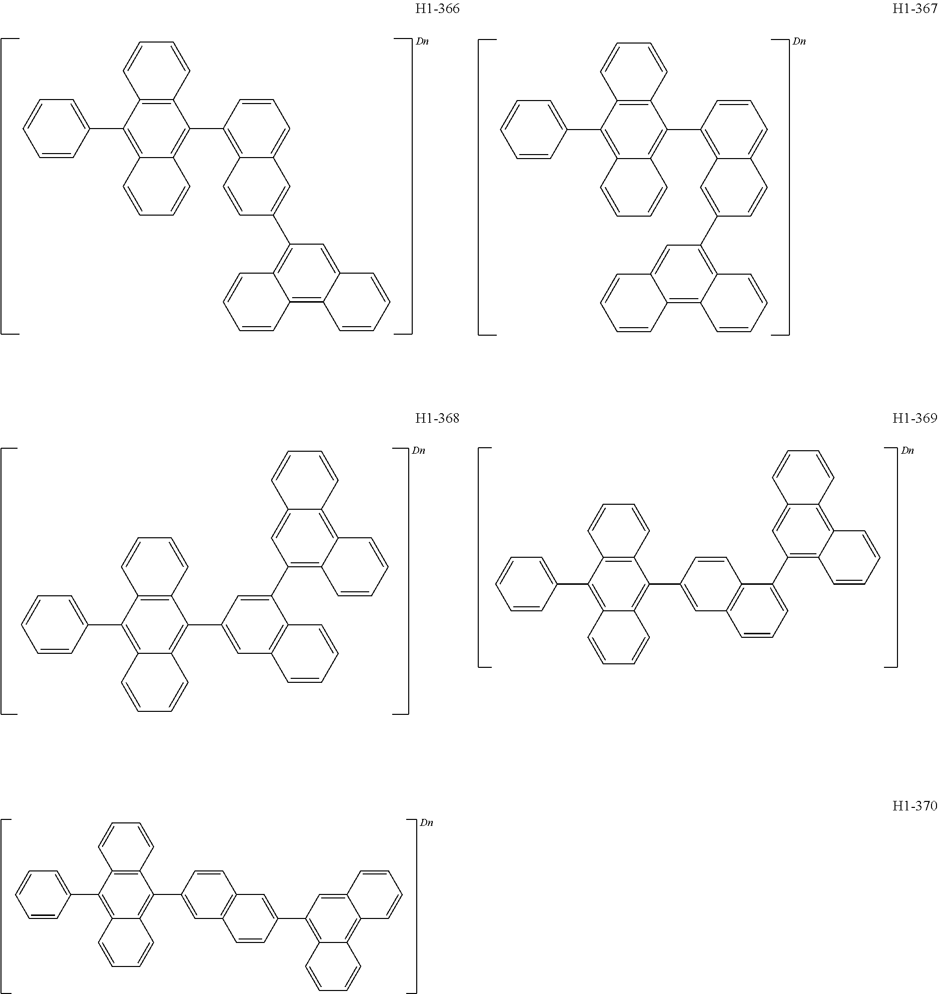

In the above compounds, Dₙ means that n hydrogens are substituted with deuterium, and n is an integer from 1 to the maximum number of hydrogens in the compound.

The compound represented by Formula 2 is described in more detail as follows.

In Formula 2, Ar₁₁ represents a substituted or unsubstituted (C₆-C₃₀)aryl or a substituted or unsubstituted (3- to 30-membered)heteroaryl. According to one embodiment of the present disclosure, Ar₁₁ may be a substituted or unsubstituted (C₆-C₂₉)aryl or a substituted or unsubstituted (3- to 25-membered)heteroaryl. According to another embodiment of the present disclosure, Ar₁₁ may be a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, a dimethylfluorenyl unsubstituted or substituted with deuterium, a diphenylfluorenyl unsubstituted or substituted with deuterium, a dimethylbenzofluorenyl unsubstituted or substituted with deuterium, a diphenylbenzofluorenyl unsubstituted or substituted with deuterium, a spirobifluorenyl unsubstituted or substituted with deuterium, a triphenylenyl unsubstituted or substituted with deuterium, a carbazolyl unsubstituted or substituted with deuterium, a benzocarbazolyl unsubstituted or substituted with deuterium, a dibenzofuranyl unsubstituted or substituted with deuterium, a benzonaphthofuranyl unsubstituted or substituted with deuterium, a dibenzothiophenyl unsubstituted or substituted with deuterium, a benzonaphthothiophenyl unsubstituted or substituted with deuterium, or a combination thereof. According to yet another embodiment of the present disclosure, Ar₁₁ may be a substituted or unsubstituted (C₆-C₂₉)aryl, or a substituted or unsubstituted (13- to 17-membered)heteroaryl. For example, Ar₁₁ may be a phenyl unsubstituted or substituted with a naphthyl(s), a dimethylfluorenyl(s), a phenanthrenyl(s), a carbazolyl(s), a benzocarbazolyl(s), a dibenzofuranyl(s), or a dibenzothiophenyl(s); a biphenyl unsubstituted or substituted with a naphthyl(s); a naphthyl unsubstituted or substituted with a phenyl(s), a biphenyl(s), or a naphthyl(s); a terphenyl; a phenanthrenyl; a dimethylfluorenyl; a diphenylfluorenyl; a dimethylbenzofluorenyl; a diphenylbenzofluorenyl; a spirobifluorenyl; a triphenylenyl; a carbazolyl unsubstituted or substituted with a phenyl(s); a benzocarbazolyl; a dibenzofuranyl; a benzonaphthofuranyl; a dibenzothiophenyl; or a benzonaphthothiophenyl, etc., which may be further substituted with deuterium.

L₁₁ represents a single bond, a substituted or unsubstituted (C₆-C₃₀)arylene, or a substituted or unsubstituted (3- to 30-membered)heteroarylene. According to one embodiment of the present disclosure, L₁₁ may be a single bond, a substituted or unsubstituted (C₆-C₂₀)arylene, or a substituted or unsubstituted (3- to 20-membered)heteroarylene. According to another embodiment of the present disclosure, L₁₁ may be a single bond, a phenylene unsubstituted or substituted with deuterium, a biphenylene unsubstituted or substituted with deuterium, a terphenylene unsubstituted or substituted with deuterium, a naphthylene unsubstituted or substituted with deuterium, or a carbazolylene unsubstituted or substituted with deuterium. According to yet another embodiment of the present disclosure, L₁₁ may be a single bond, a substituted or unsubstituted (C₆-C₁₂)arylene, or a substituted or unsubstituted (6- to 20-membered)heteroarylene. For example, L₁₁ may be a single bond, a phenylene, a biphenylene, a naphthylene, or a carbazolylene, etc., which may be further substituted with deuterium.

R₁₁ to R₁₈ each independently represent hydrogen or deuterium.

L₁₂ represents a single bond or a substituted or unsubstituted (C₆-C₃₀)arylene. According to one embodiment of the present disclosure, L₁₂ may be a (C₆-C₁₂)arylene unsubstituted or substituted with deuterium. According to another embodiment of the present disclosure, L₁₂ may be a single bond, a phenylene unsubstituted or substituted with deuterium, a biphenylene unsubstituted or substituted with deuterium, a terphenylene unsubstituted or substituted with deuterium, a naphthylene unsubstituted or substituted with deuterium, or a carbazolylene unsubstituted or substituted with deuterium. According to yet another embodiment of the present disclosure, L₁₂ may be a phenylene unsubstituted or substituted with deuterium. For example, L₁₂ may be a single bond, a phenylene, a biphenylene, or a naphthylene, etc., which may be further substituted with deuterium.

Ar₁₂ is represented by the following Formula 2-1.

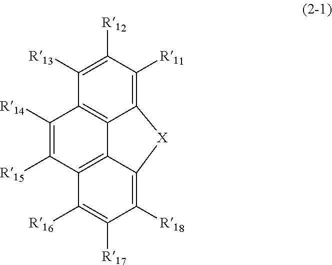

In Formula 2-1, X is O or S.

In Formula 2-1, R′₁₁ to R′₁₈ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a combination thereof; or may be linked to the adjacent substituents to form a ring(s). According to one embodiment of the present disclosure, R′₁₁ to R′₁₈ may each independently be hydrogen, deuterium, a substituted or unsubstituted (C₆-C₃₀)aryl, or a combination thereof; or may be linked to the adjacent substituents to form a ring(s). According to another embodiment of the present disclosure, R′₁₁ to R′₁₈ may each independently be hydrogen, deuterium, a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof; or may be linked to the adjacent substituents to form a benzene ring(s) unsubstituted or substituted with deuterium. For example, R′₁₁ to R′₁₈ may each independently be hydrogen, deuterium, a phenyl, a biphenyl, or a naphthyl, etc.; or may be linked to the adjacent substituents to form a benzene ring(s), which may be further substituted with deuterium.

One of R′₁₁ to R′₁₈ is linked to L₁₂.

According to one embodiment of the present disclosure, the compound represented by Formula 2 may be at least one selected from the following compounds, but is not limited thereto.

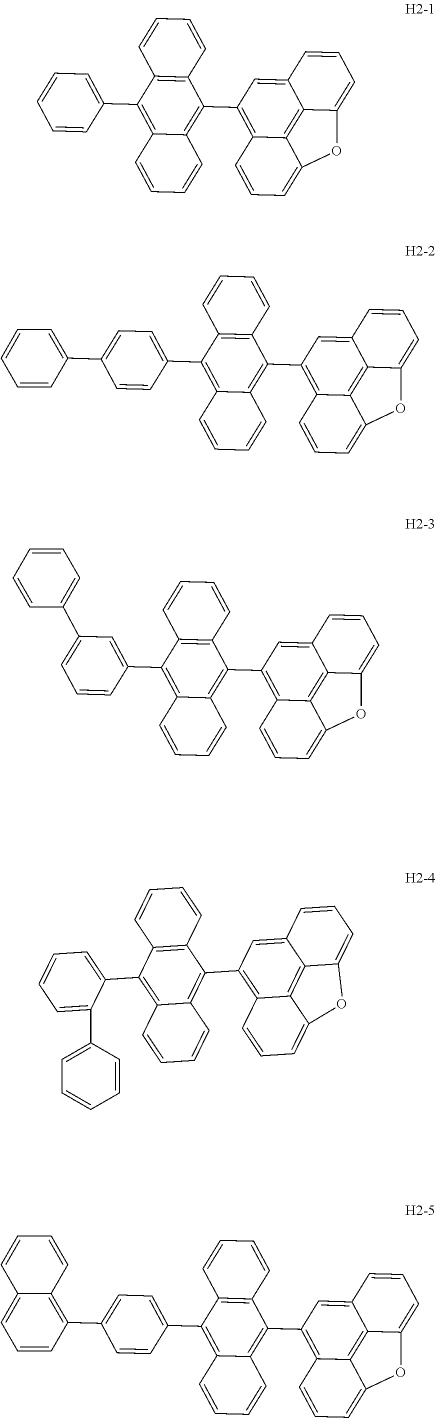 [SMILES: c1ccc(-c2c3ccccc3c(-c3cc4cccc5oc6cccc3c6c45)c3ccccc23)cc1 ; c1ccc(-c2ccc(-c3c4ccccc4c(-c4cc5cccc6oc7cccc4c7c56)c4ccccc34)cc2)cc1 ; c1ccc(-c2cccc(-c3c4ccccc4c(-c4cc5cccc6oc7cccc4c7c56)c4ccccc34)c2)cc1 ; c1ccc(-c2ccccc2-c2c3ccccc3c(-c3cc4cccc5oc6cccc3c6c45)c3ccccc23)cc1 ; c1ccc2c(-c3ccc(-c4c5ccccc5c(-c5cc6cccc7oc8cccc5c8c67)c5ccccc45)cc3)cccc2c1]

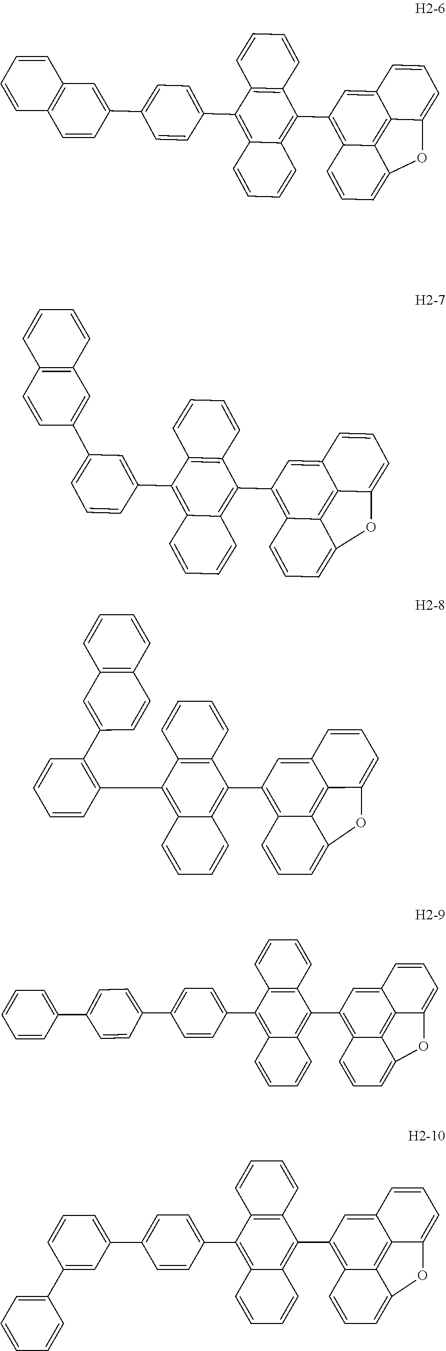 [SMILES: c1ccc2cc(-c3ccc(-c4c5ccccc5c(-c5cc6cccc7oc8cccc5c8c67)c5ccccc45)cc3)ccc2c1 ; c1cc(-c2ccc3ccccc3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3ccccc3c2)c1 ; c1ccc(-c2ccc(-c3ccc(-c4c5ccccc5c(-c5cc6cccc7oc8cccc5c8c67)c5ccccc45)cc3)cc2)cc1 ; c1ccc(-c2cccc(-c3ccc(-c4c5ccccc5c(-c5cc6cccc7oc8cccc5c8c67)c5ccccc45)cc3)c2)cc1]

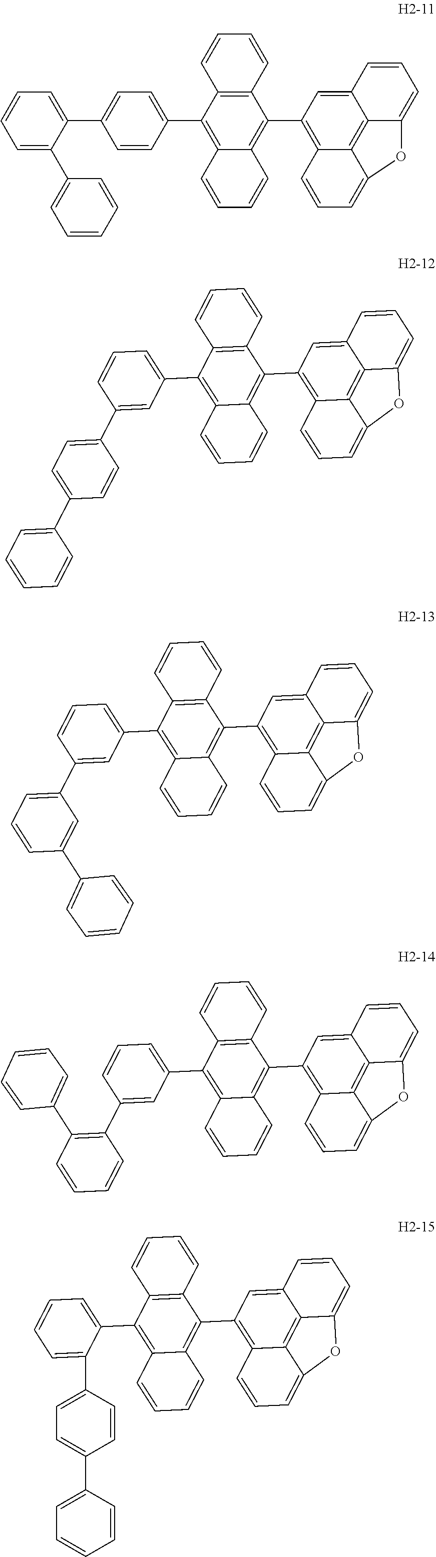 [SMILES: c1ccc(-c2ccccc2-c2ccc(-c3c4ccccc4c(-c4cc5cccc6oc7cccc4c7c56)c4ccccc34)cc2)cc1 ; c1ccc(-c2ccc(-c3cccc(-c4c5ccccc5c(-c5cc6cccc7oc8cccc5c8c67)c5ccccc45)c3)cc2)cc1 ; c1ccc(-c2cccc(-c3cccc(-c4c5ccccc5c(-c5cc6cccc7oc8cccc5c8c67)c5ccccc45)c3)c2)cc1 ; c1ccc(-c2ccccc2-c2cccc(-c3c4ccccc4c(-c4cc5cccc6oc7cccc4c7c56)c4ccccc34)c2)cc1 ; c1ccc(-c2ccc(-c3ccccc3-c3c4ccccc4c(-c4cc5cccc6oc7cccc4c7c56)c4ccccc34)cc2)cc1]

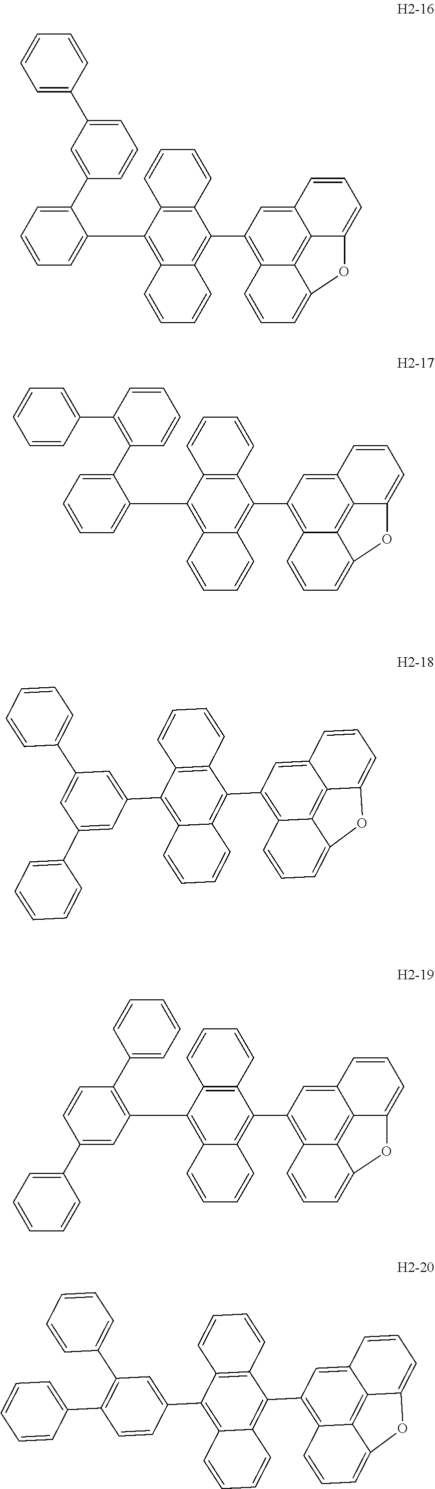 [SMILES: c1ccc(-c2cccc(-c3ccccc3-c3c4ccccc4c(-c4cc5cccc6oc7cccc4c7c56)c4ccccc34)c2)cc1 ; c1ccc(-c2ccccc2-c2ccccc2-c2c3ccccc3c(-c3cc4cccc5oc6cccc3c6c45)c3ccccc23)cc1 ; c1ccc(-c2cc(-c3ccccc3)cc(-c3c4ccccc4c(-c4cc5cccc6oc7cccc4c7c56)c4ccccc34)c2)cc1 ; c1ccc(-c2ccc(-c3ccccc3)c(-c3c4ccccc4c(-c4cc5cccc6oc7cccc4c7c56)c4ccccc34)c2)cc1 ; c1ccc(-c2ccc(-c3c4ccccc4c(-c4cc5cccc6oc7cccc4c7c56)c4ccccc34)cc2-c2ccccc2)cc1]

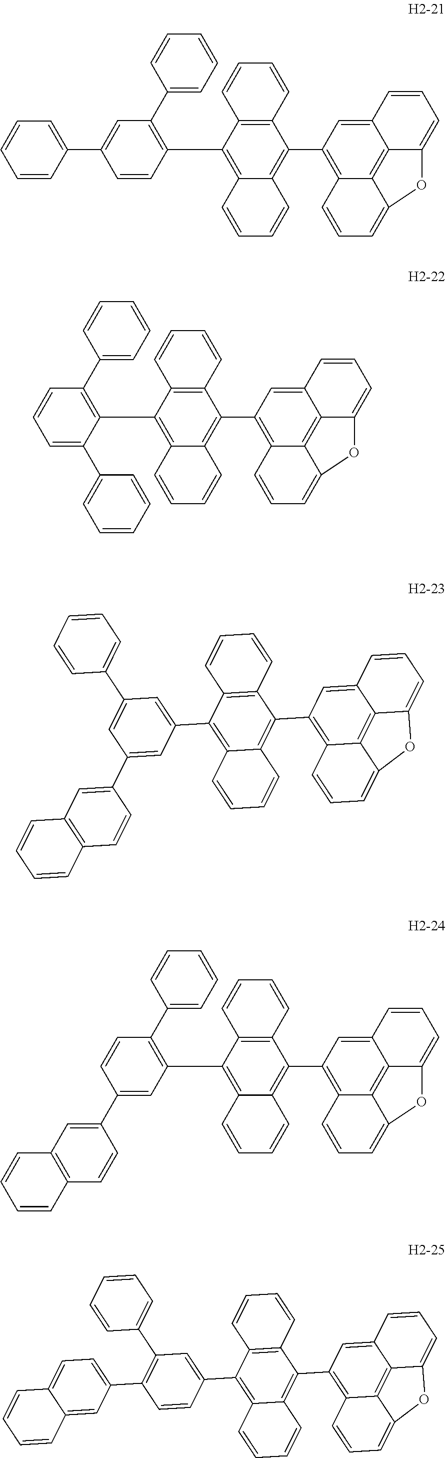 [SMILES: c1ccc(-c2ccc(-c3c4ccccc4c(-c4cc5cccc6oc7cccc4c7c56)c4ccccc34)c(-c3ccccc3)c2)cc1 ; c1ccc(-c2cccc(-c3ccccc3)c2-c2c3ccccc3c(-c3cc4cccc5oc6cccc3c6c45)c3ccccc23)cc1 ; c1ccc(-c2cc(-c3ccc4ccccc4c3)cc(-c3c4ccccc4c(-c4cc5cccc6oc7cccc4c7c56)c4ccccc34)c2)cc1 ; c1ccc(-c2ccc(-c3ccc4ccccc4c3)cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)ccc2-c2ccc3ccccc3c2)cc1]

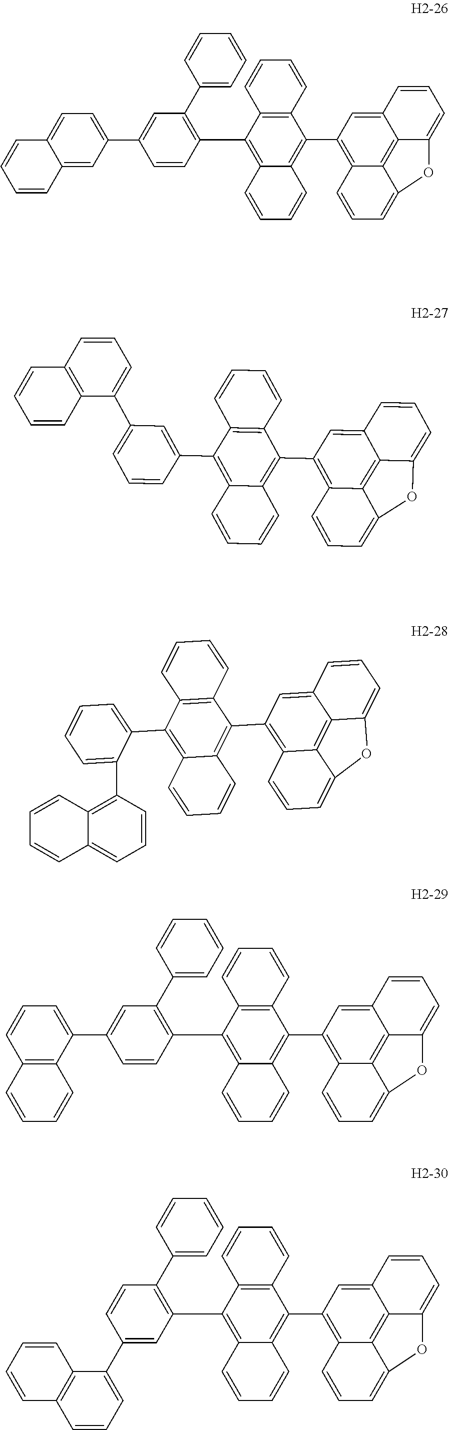 [SMILES: c1ccc(-c2cc(-c3ccc4ccccc4c3)ccc2-c2c3ccccc3c(-c3cc4cccc5oc6cccc3c6c45)c3ccccc23)cc1 ; c1cc(-c2cccc3ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2cccc3ccccc23)c1 ; c1ccc(-c2cc(-c3cccc4ccccc34)ccc2-c2c3ccccc3c(-c3cc4cccc5oc6cccc3c6c45)c3ccccc23)cc1 ; c1ccc(-c2ccc(-c3cccc4ccccc34)cc2-c2c3ccccc3c(-c3cc4cccc5oc6cccc3c6c45)c3ccccc23)cc1]

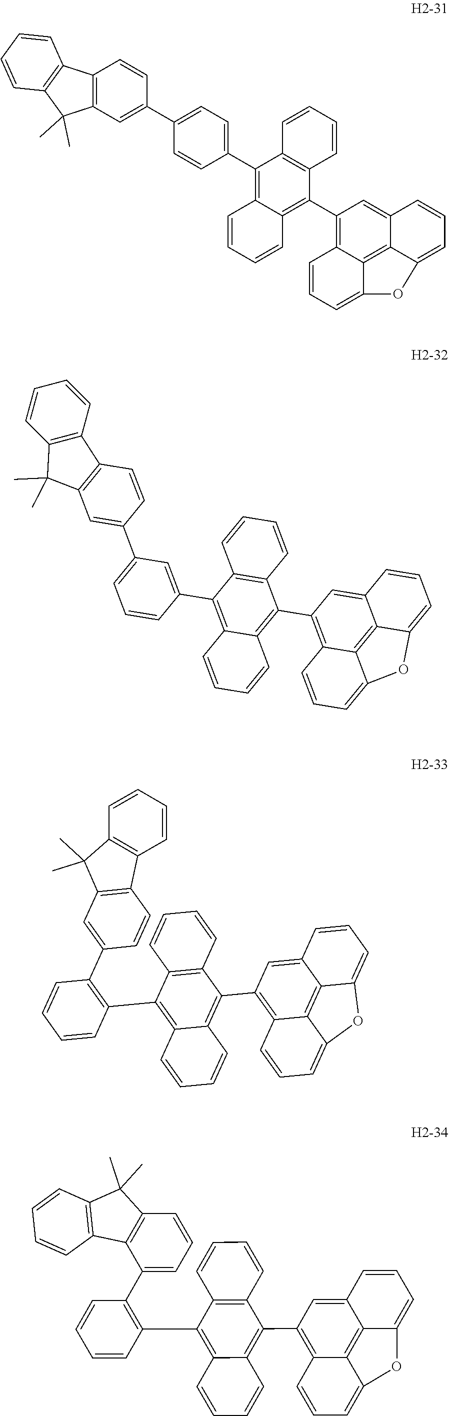 [SMILES: CC1(C)c2ccccc2-c2ccc(-c3ccc(-c4c5ccccc5c(-c5cc6cccc7oc8cccc5c8c67)c5ccccc45)cc3)cc21 ; CC1(C)c2ccccc2-c2ccc(-c3cccc(-c4c5ccccc5c(-c5cc6cccc7oc8cccc5c8c67)c5ccccc45)c3)cc21 ; CC1(C)c2ccccc2-c2ccc(-c3ccccc3-c3c4ccccc4c(-c4cc5cccc6oc7cccc4c7c56)c4ccccc34)cc21 ; CC1(C)c2ccccc2-c2c(-c3ccccc3-c3c4ccccc4c(-c4cc5cccc6oc7cccc4c7c56)c4ccccc34)cccc21]

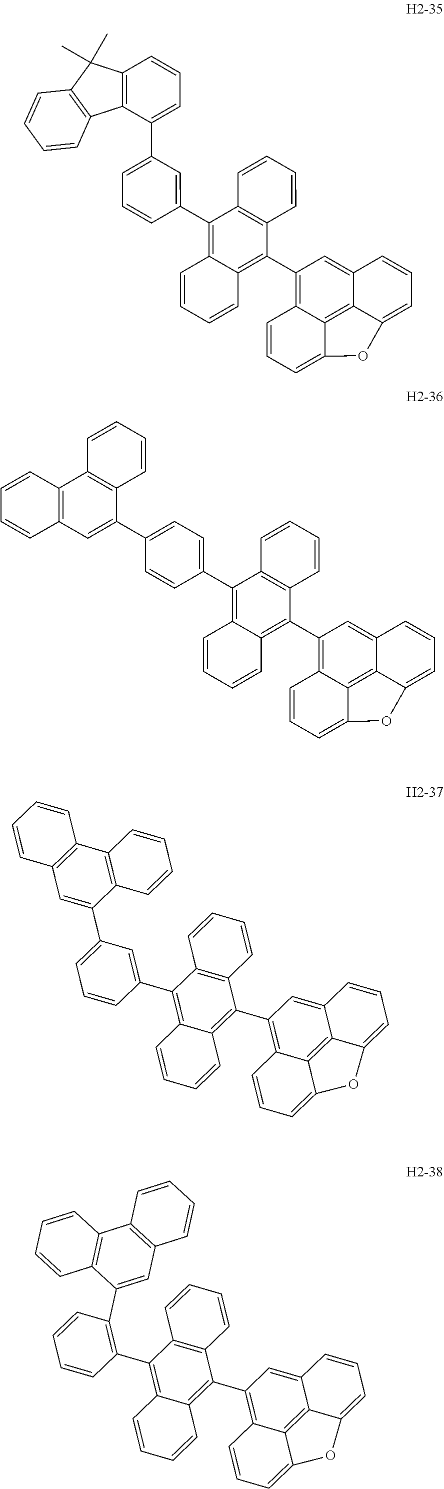 [SMILES: CC1(C)c2ccccc2-c2c(-c3cccc(-c4c5ccccc5c(-c5cc6cccc7oc8cccc5c8c67)c5ccccc45)c3)cccc21 ; c1ccc2c(c1)cc(-c1ccc(-c3c4ccccc4c(-c4cc5cccc6oc7cccc4c7c56)c4ccccc34)cc1)c1ccccc12 ; c1cc(-c2c3ccccc3c(-c3cc4cccc5oc6cccc3c6c45)c3ccccc23)cc(-c2cc3ccccc3c3ccccc23)c1 ; c1ccc(-c2cc3ccccc3c3ccccc23)c(-c2c3ccccc3c(-c3cc4cccc5oc6cccc3c6c45)c3ccccc23)c1]

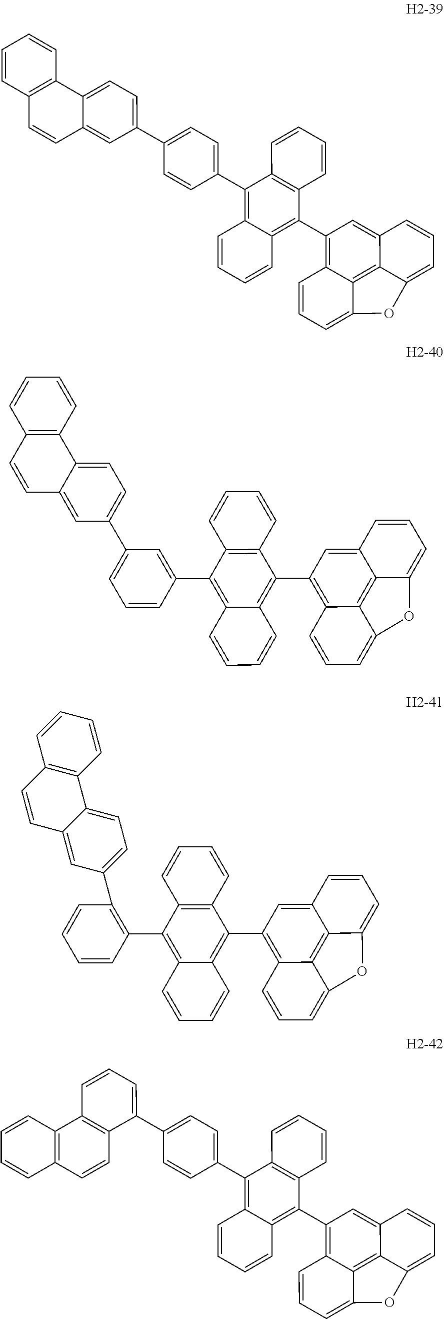 [SMILES: c1ccc2c(c1)ccc1cc(-c3ccc(-c4c5ccccc5c(-c5cc6cccc7oc8cccc5c8c67)c5ccccc45)cc3)ccc12 ; c1cc(-c2ccc3c(ccc4ccccc43)c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(ccc4ccccc43)c2)c1 ; c1ccc2c(c1)ccc1c(-c3ccc(-c4c5ccccc5c(-c5cc6cccc7oc8cccc5c8c67)c5ccccc45)cc3)cccc12]

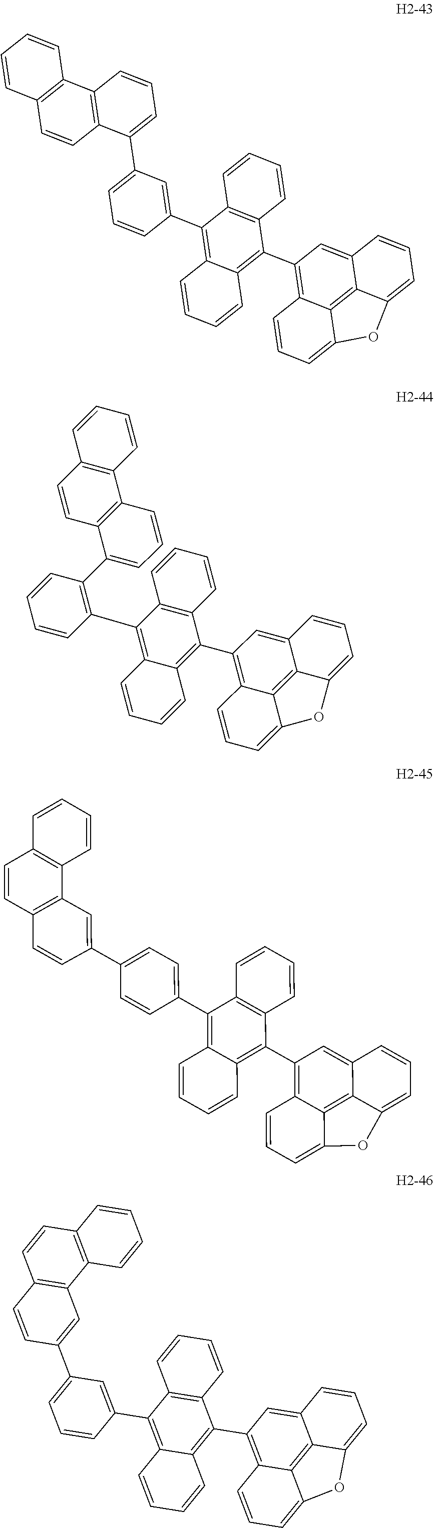 [SMILES: c1cc(-c2c3ccccc3c(-c3cc4cccc5oc6cccc3c6c45)c3ccccc23)cc(-c2cccc3c2ccc2ccccc23)c1 ; c1ccc(-c2cccc3c2ccc2ccccc23)c(-c2c3ccccc3c(-c3cc4cccc5oc6cccc3c6c45)c3ccccc23)c1 ; c1ccc2c(c1)ccc1ccc(-c3ccc(-c4c5ccccc5c(-c5cc6cccc7oc8cccc5c8c67)c5ccccc45)cc3)cc12 ; c1cc(-c2ccc3ccc4ccccc4c3c2)cc(-c2c3ccccc3c(-c3cc4cccc5oc6cccc3c6c45)c3ccccc23)c1]

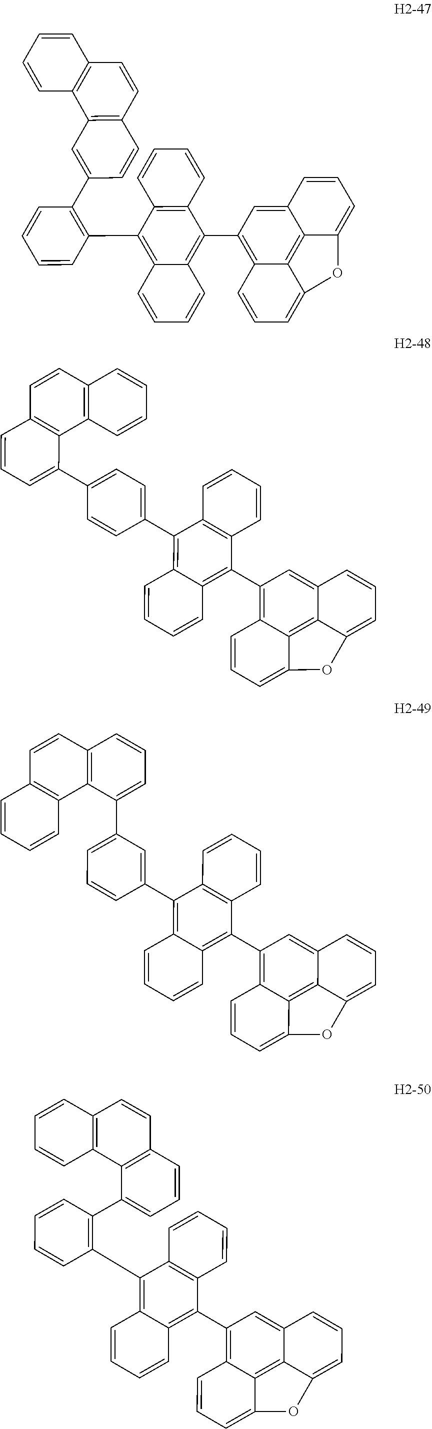 [SMILES: c1ccc(-c2c3ccccc3c(-c3cc4cccc5oc6cccc3c6c45)c3ccccc23)c(-c2ccc3ccc4ccccc4c3c2)c1 ; c1ccc2c(c1)ccc1cccc(-c3ccc(-c4c5ccccc5c(-c5cc6cccc7oc8cccc5c8c67)c5ccccc45)cc3)c12 ; c1cc(-c2c3ccccc3c(-c3cc4cccc5oc6cccc3c6c45)c3ccccc23)cc(-c2cccc3ccc4ccccc4c23)c1 ; c1ccc(-c2cccc3ccc4ccccc4c23)c(-c2c3ccccc3c(-c3cc4cccc5oc6cccc3c6c45)c3ccccc23)c1]

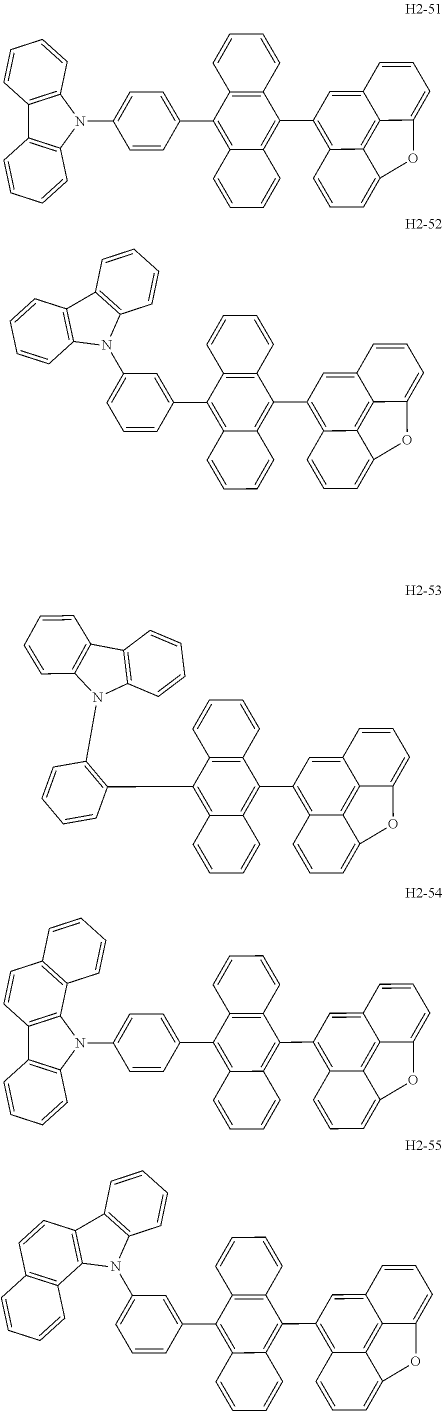 [SMILES: c1cc2cc(-c3c4ccccc4c(-c4ccc(-n5c6ccccc6c6ccccc65)cc4)c4ccccc34)c3cccc4oc(c1)c2c43 ; c1cc(-c2c3ccccc3c(-c3cc4cccc5oc6cccc3c6c45)c3ccccc23)cc(-n2c3ccccc3c3ccccc32)c1 ; c1ccc(-n2c3ccccc3c3ccccc32)c(-c2c3ccccc3c(-c3cc4cccc5oc6cccc3c6c45)c3ccccc23)c1 ; c1ccc2c(c1)ccc1c3ccccc3n(-c3ccc(-c4c5ccccc5c(-c5cc6cccc7oc8cccc5c8c67)c5ccccc45)cc3)c21 ; c1cc(-c2c3ccccc3c(-c3cc4cccc5oc6cccc3c6c45)c3ccccc23)cc(-n2c3ccccc3c3ccc4ccccc4c32)c1]

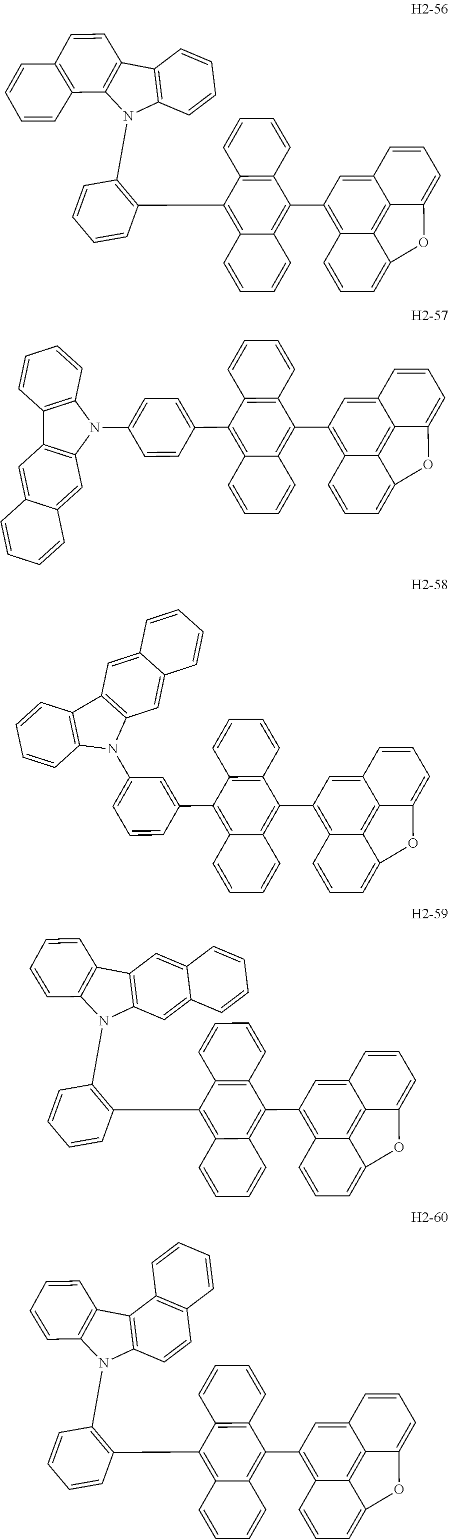 [SMILES: c1ccc(-n2c3ccccc3c3ccc4ccccc4c32)c(-c2c3ccccc3c(-c3cc4cccc5oc6cccc3c6c45)c3ccccc23)c1 ; c1ccc2cc3c(cc2c1)c1ccccc1n3-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3cc4ccccc4cc32)c1 ; c1ccc(-n2c3ccccc3c3cc4ccccc4cc32)c(-c2c3ccccc3c(-c3cc4cccc5oc6cccc3c6c45)c3ccccc23)c1 ; c1ccc(-n2c3ccccc3c3c4ccccc4ccc32)c(-c2c3ccccc3c(-c3cc4cccc5oc6cccc3c6c45)c3ccccc23)c1]

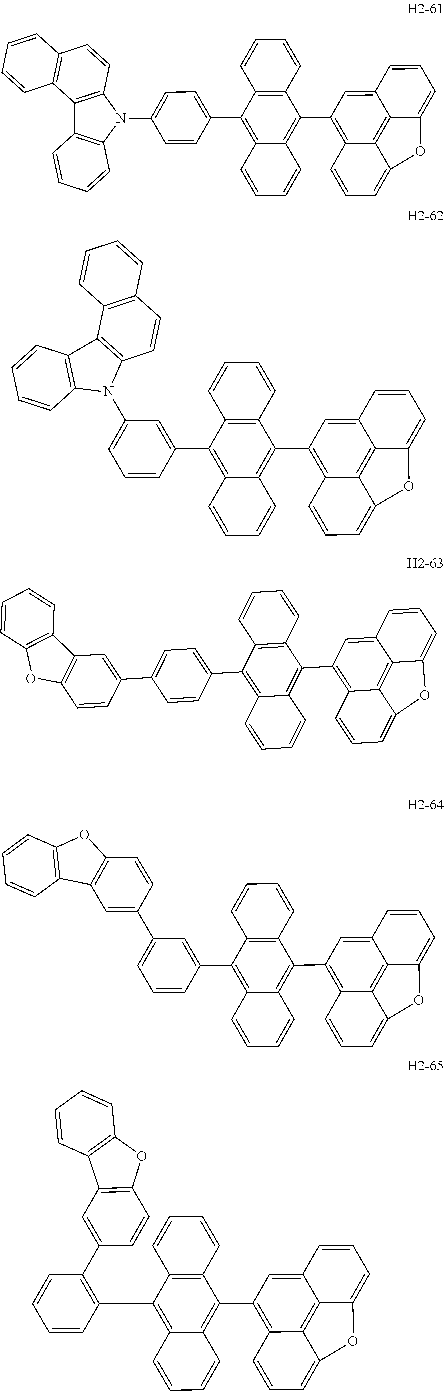 [SMILES: c1ccc2c(c1)ccc1c2c2ccccc2n1-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3c4ccccc4ccc32)c1 ; c1ccc2c(c1)oc1ccc(-c3ccc(-c4c5ccccc5c(-c5cc6cccc7oc8cccc5c8c67)c5ccccc45)cc3)cc12 ; c1cc(-c2ccc3oc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3oc4ccccc4c3c2)c1]

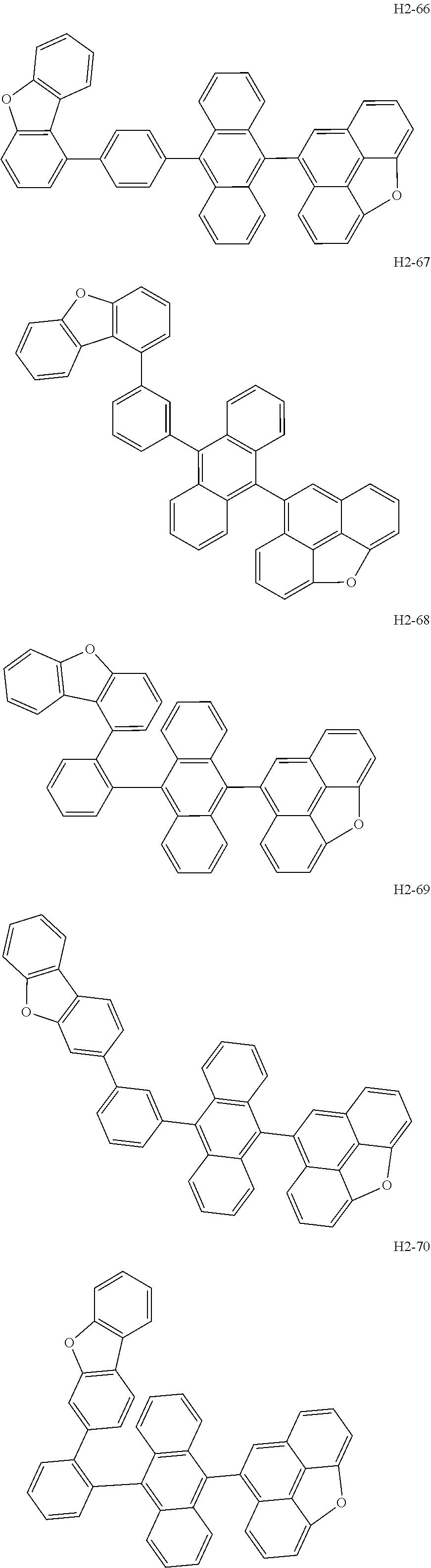 [SMILES: c1ccc2c(c1)oc1cccc(-c3ccc(-c4c5ccccc5c(-c5cc6cccc7oc8cccc5c8c67)c5ccccc45)cc3)c12 ; c1cc(-c2c3ccccc3c(-c3cc4cccc5oc6cccc3c6c45)c3ccccc23)cc(-c2cccc3oc4ccccc4c23)c1 ; c1ccc(-c2cccc3oc4ccccc4c23)c(-c2c3ccccc3c(-c3cc4cccc5oc6cccc3c6c45)c3ccccc23)c1 ; c1cc(-c2ccc3c(c2)oc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)oc2ccccc23)c1]

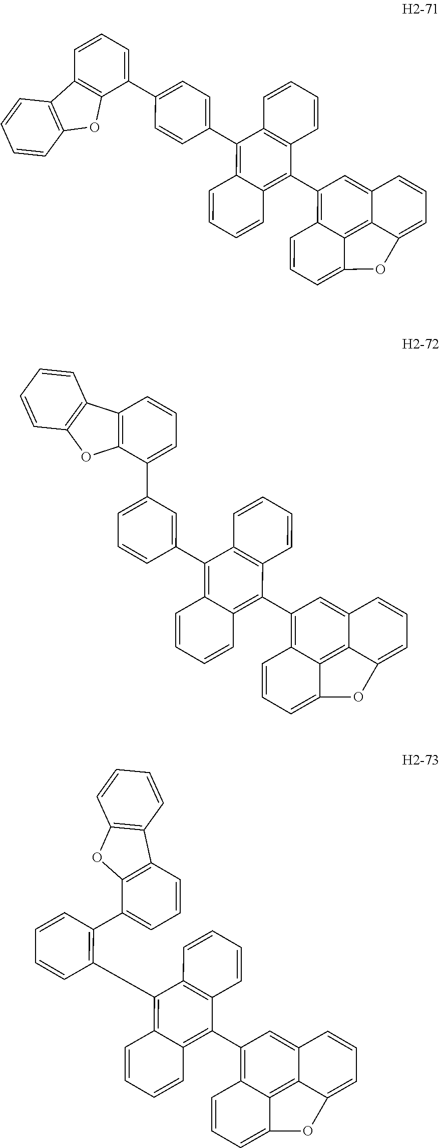 [SMILES: c1ccc2c(c1)oc1c(-c3ccc(-c4c5ccccc5c(-c5cc6cccc7oc8cccc5c8c67)c5ccccc45)cc3)cccc12 ; c1cc(-c2c3ccccc3c(-c3cc4cccc5oc6cccc3c6c45)c3ccccc23)cc(-c2cccc3c2oc2ccccc23)c1 ; c1ccc(-c2cccc3c2oc2ccccc23)c(-c2c3ccccc3c(-c3cc4cccc5oc6cccc3c6c45)c3ccccc23)c1]

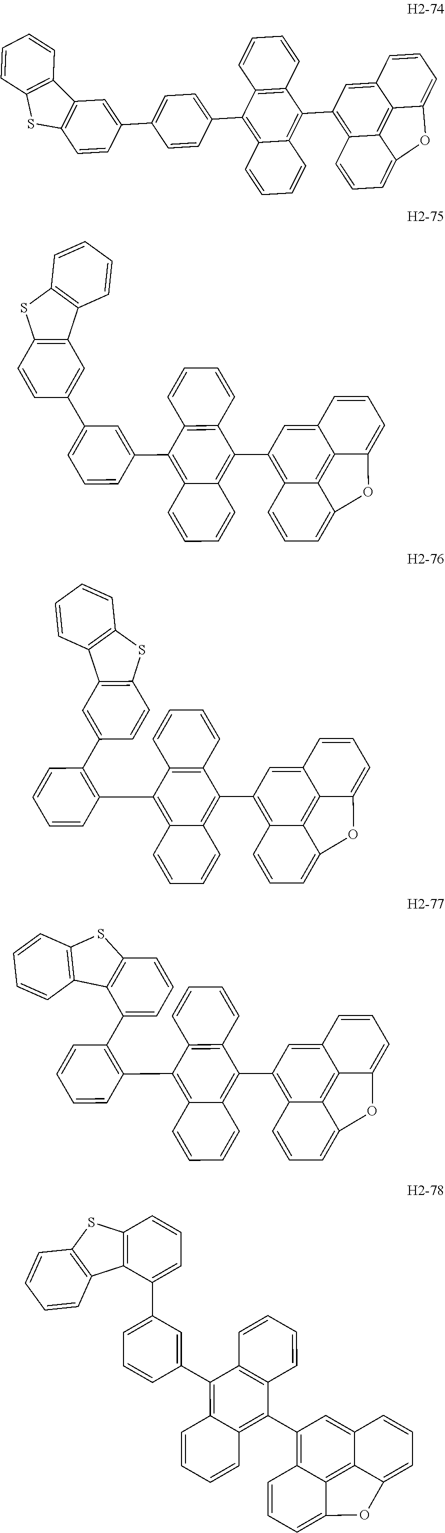 [SMILES: c1cc2cc(-c3c4ccccc4c(-c4ccc(-c5ccc6sc7ccccc7c6c5)cc4)c4ccccc34)c3cccc4oc(c1)c2c43 ; c1cc(-c2ccc3sc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3sc4ccccc4c3c2)c1 ; c1ccc(-c2cccc3sc4ccccc4c23)c(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-c2cccc3sc4ccccc4c23)c1]

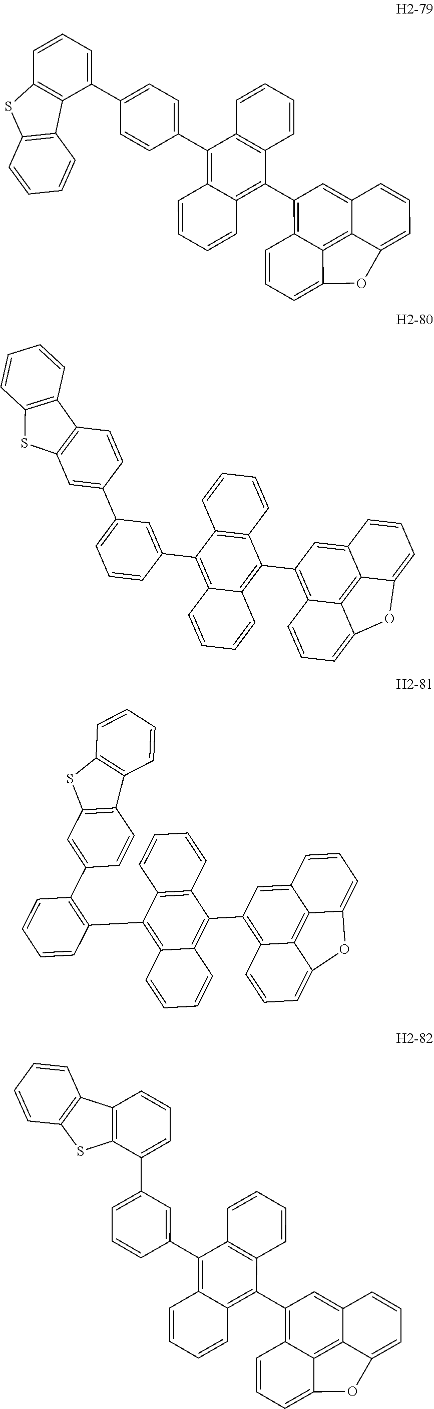 [SMILES: c1cc2cc(-c3c4ccccc4c(-c4ccc(-c5cccc6sc7ccccc7c56)cc4)c4ccccc34)c3cccc4oc(c1)c2c43 ; c1cc(-c2ccc3c(c2)sc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)sc2ccccc23)c1 ; c1cc(-c2c3ccccc3c(-c3cc4cccc5oc6cccc3c6c45)c3ccccc23)cc(-c2cccc3c2sc2ccccc23)c1]

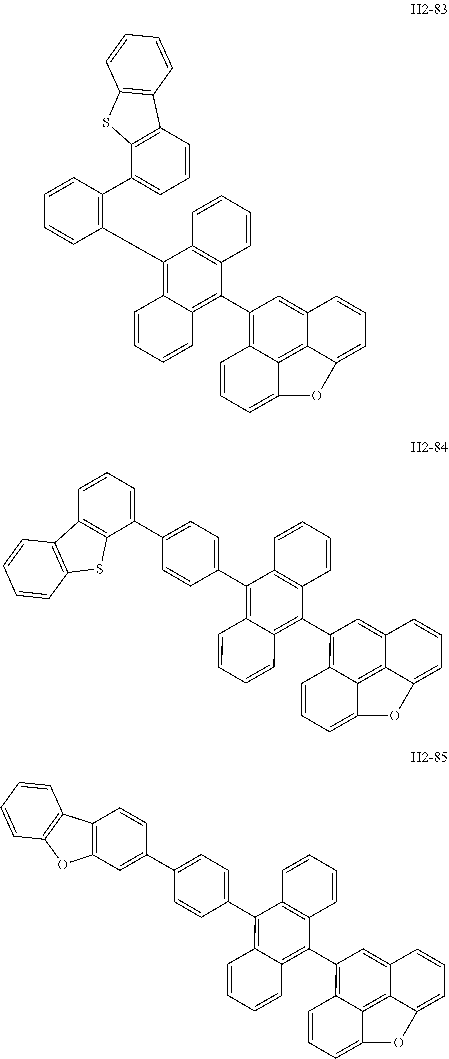 [SMILES: c1ccc(-c2cccc3c2sc2ccccc23)c(-c2c3ccccc3c(-c3cc4cccc5oc6cccc3c6c45)c3ccccc23)c1 ; c1cc2cc(-c3c4ccccc4c(-c4ccc(-c5cccc6c5sc5ccccc56)cc4)c4ccccc34)c3cccc4oc(c1)c2c43 ; c1ccc2c(c1)oc1cc(-c3ccc(-c4c5ccccc5c(-c5cc6cccc7oc8cccc5c8c67)c5ccccc45)cc3)ccc12]

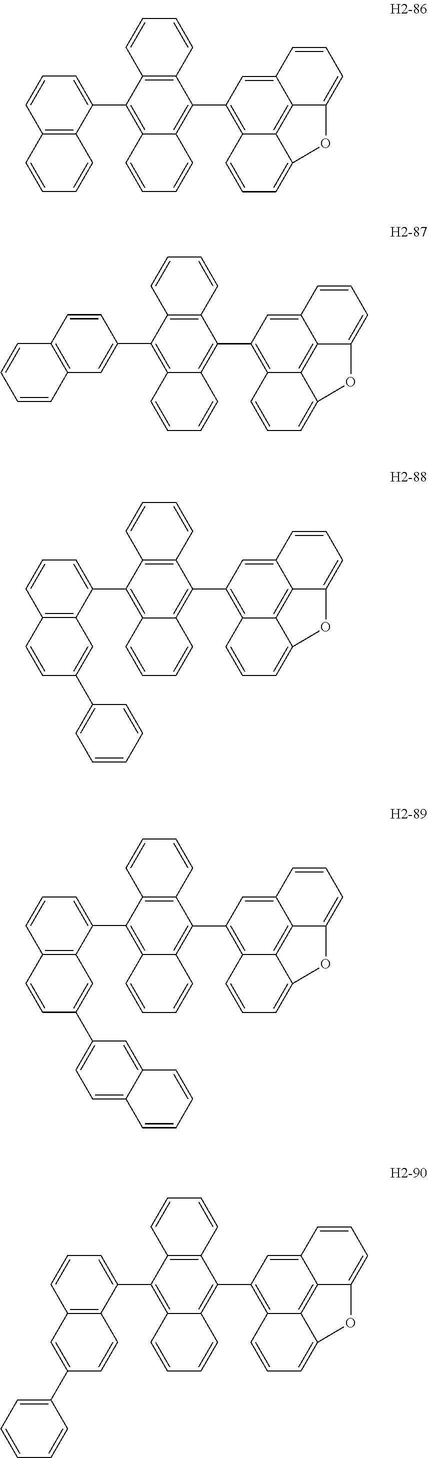 [SMILES: c1ccc2c(-c3c4ccccc4c(-c4cc5cccc6oc7cccc4c7c56)c4ccccc34)cccc2c1 ; c1ccc2cc(-c3c4ccccc4c(-c4cc5cccc6oc7cccc4c7c56)c4ccccc34)ccc2c1 ; c1ccc(-c2ccc3cccc(-c4c5ccccc5c(-c5cc6cccc7oc8cccc5c8c67)c5ccccc45)c3c2)cc1 ; c1ccc2cc(-c3ccc4cccc(-c5c6ccccc6c(-c6cc7cccc8oc9cccc6c9c78)c6ccccc56)c4c3)ccc2c1 ; c1ccc(-c2ccc3c(-c4c5ccccc5c(-c5cc6cccc7oc8cccc5c8c67)c5ccccc45)cccc3c2)cc1]

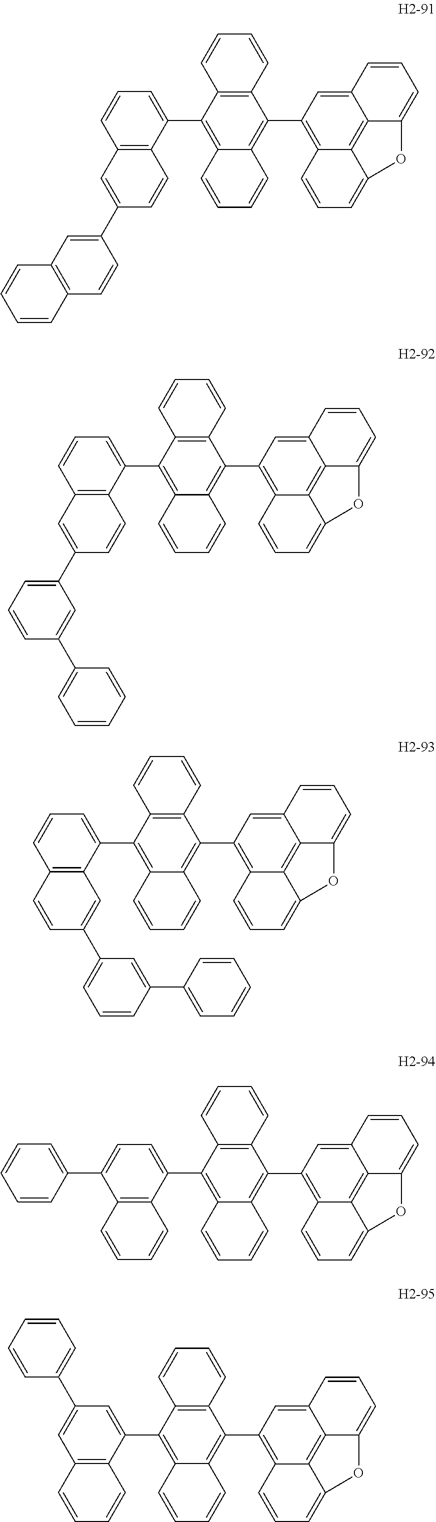 [SMILES: c1ccc2cc(-c3ccc4c(-c5c6ccccc6c(-c6cc7cccc8oc9cccc6c9c78)c6ccccc56)cccc4c3)ccc2c1 ; c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1 ; c1ccc(-c2ccc(-c3c4ccccc4c(-c4cc5cccc6oc7cccc4c7c56)c4ccccc34)c3ccccc23)cc1 ; c1ccc(-c2cc(-c3c4ccccc4c(-c4cc5cccc6oc7cccc4c7c56)c4ccccc34)c3ccccc3c2)cc1]

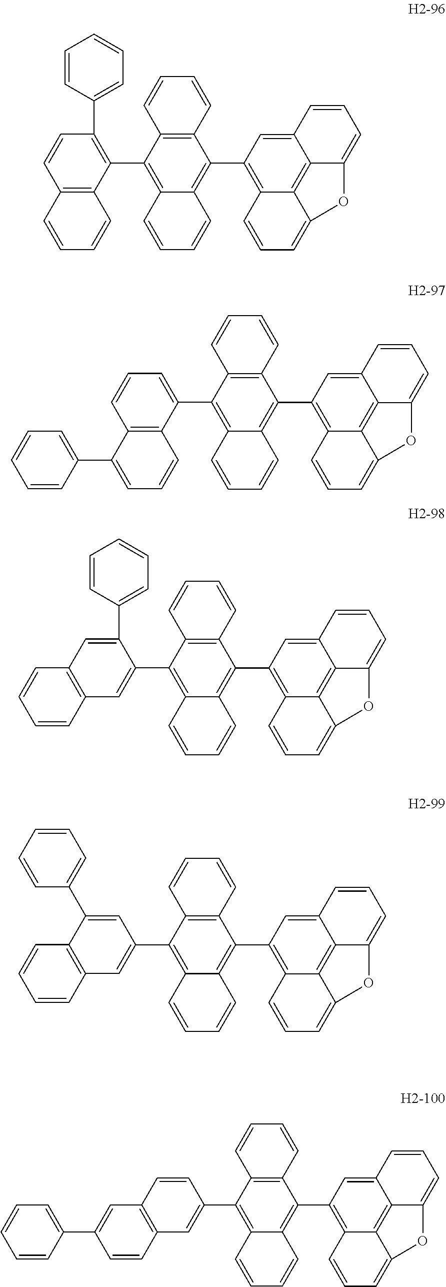 [SMILES: c1ccc(-c2ccc3ccccc3c2-c2c3ccccc3c(-c3cc4cccc5oc6cccc3c6c45)c3ccccc23)cc1 ; c1ccc(-c2cccc3c(-c4c5ccccc5c(-c5cc6cccc7oc8cccc5c8c67)c5ccccc45)cccc23)cc1 ; c1ccc(-c2cc3ccccc3cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)cc3ccccc23)cc1 ; c1ccc(-c2ccc3cc(-c4c5ccccc5c(-c5cc6cccc7oc8cccc5c8c67)c5ccccc45)ccc3c2)cc1]

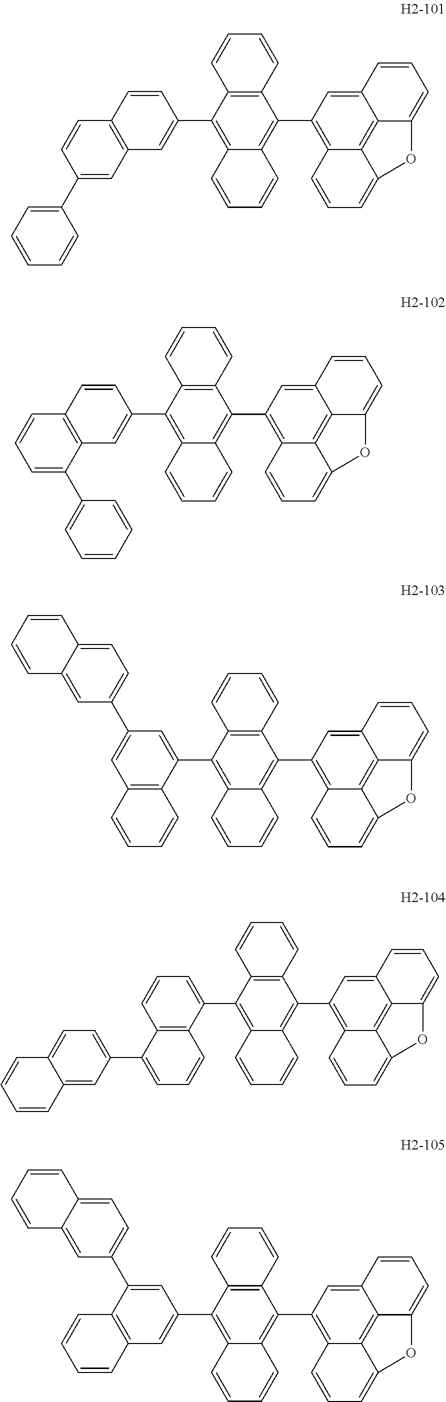 [SMILES: c1ccc(-c2ccc3ccc(-c4c5ccccc5c(-c5cc6cccc7oc8cccc5c8c67)c5ccccc45)cc3c2)cc1 ; c1ccc(-c2cccc3ccc(-c4c5ccccc5c(-c5cc6cccc7oc8cccc5c8c67)c5ccccc45)cc23)cc1 ; c1ccc2cc(-c3cc(-c4c5ccccc5c(-c5cc6cccc7oc8cccc5c8c67)c5ccccc45)c4ccccc4c3)ccc2c1 ; c1ccc2cc(-c3cccc4c(-c5c6ccccc6c(-c6cc7cccc8oc9cccc6c9c78)c6ccccc56)cccc34)ccc2c1 ; c1ccc2cc(-c3cc(-c4c5ccccc5c(-c5cc6cccc7oc8cccc5c8c67)c5ccccc45)cc4ccccc34)ccc2c1]

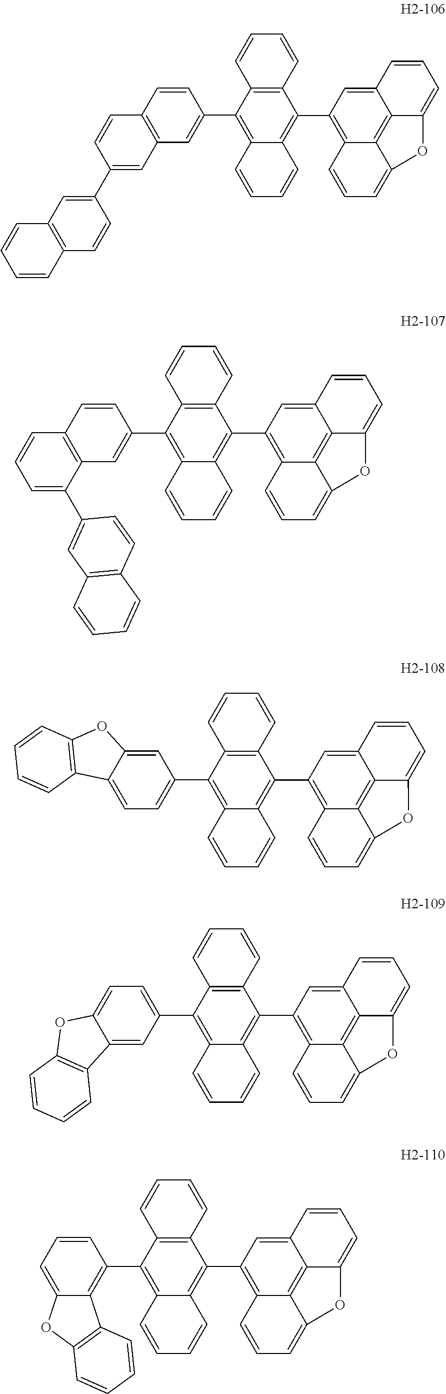 [SMILES: c1ccc2cc(-c3ccc4ccc(-c5c6ccccc6c(-c6cc7cccc8oc9cccc6c9c78)c6ccccc56)cc4c3)ccc2c1 ; c1ccc2cc(-c3cccc4ccc(-c5c6ccccc6c(-c6cc7cccc8oc9cccc6c9c78)c6ccccc56)cc34)ccc2c1 ; c1ccc2c(c1)oc1cc(-c3c4ccccc4c(-c4cc5cccc6oc7cccc4c7c56)c4ccccc34)ccc12 ; c1ccc2c(c1)oc1ccc(-c3c4ccccc4c(-c4cc5cccc6oc7cccc4c7c56)c4ccccc34)cc12 ; c1ccc2c(c1)oc1cccc(-c3c4ccccc4c(-c4cc5cccc6oc7cccc4c7c56)c4ccccc34)c12]

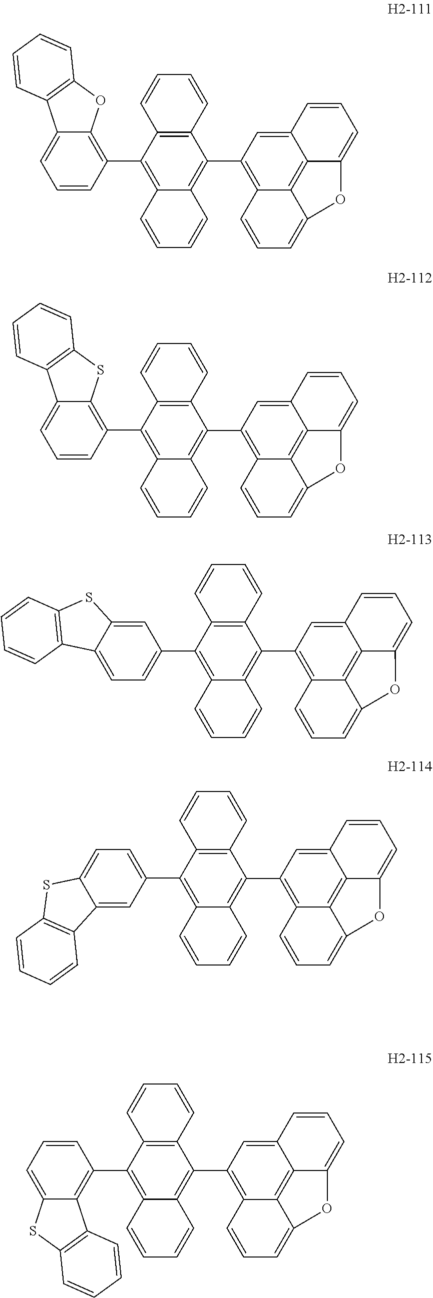 [SMILES: c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43 ; c1cc2cc(-c3c4ccccc4c(-c4ccc5sc6ccccc6c5c4)c4ccccc34)c3cccc4oc(c1)c2c43 ; c1cc2cc(-c3c4ccccc4c(-c4cccc5sc6ccccc6c45)c4ccccc34)c3cccc4oc(c1)c2c43]

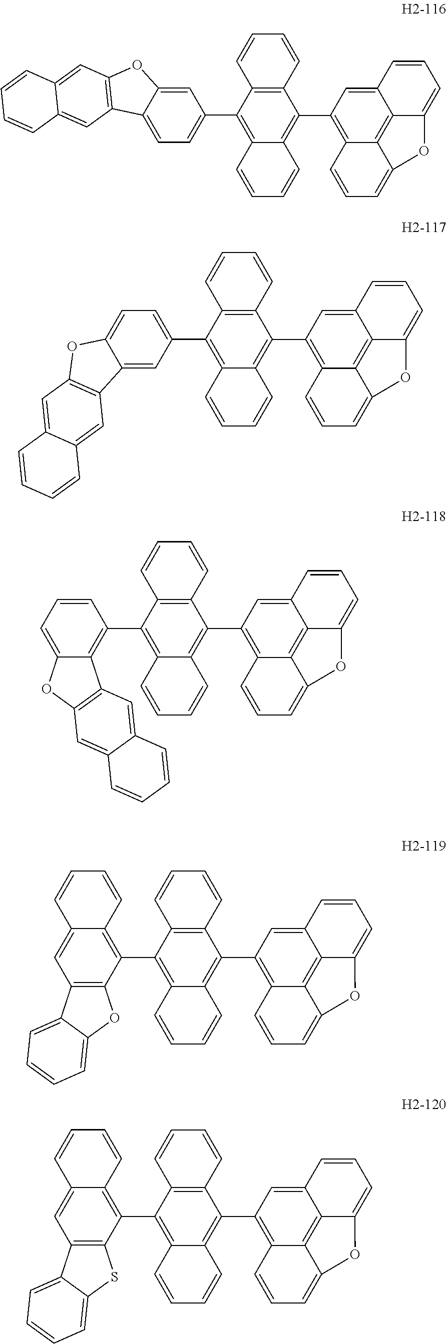 [SMILES: c1ccc2cc3c(cc2c1)oc1cc(-c2c4ccccc4c(-c4cc5cccc6oc7cccc4c7c56)c4ccccc24)ccc13 ; c1ccc2cc3c(cc2c1)oc1ccc(-c2c4ccccc4c(-c4cc5cccc6oc7cccc4c7c56)c4ccccc24)cc13 ; c1ccc2cc3c(cc2c1)oc1cccc(-c2c4ccccc4c(-c4cc5cccc6oc7cccc4c7c56)c4ccccc24)c13 ; c1ccc2c(-c3c4ccccc4c(-c4cc5cccc6oc7cccc4c7c56)c4ccccc34)c3oc4ccccc4c3cc2c1 ; c1ccc2c(-c3c4ccccc4c(-c4cc5cccc6oc7cccc4c7c56)c4ccccc34)c3sc4ccccc4c3cc2c1]

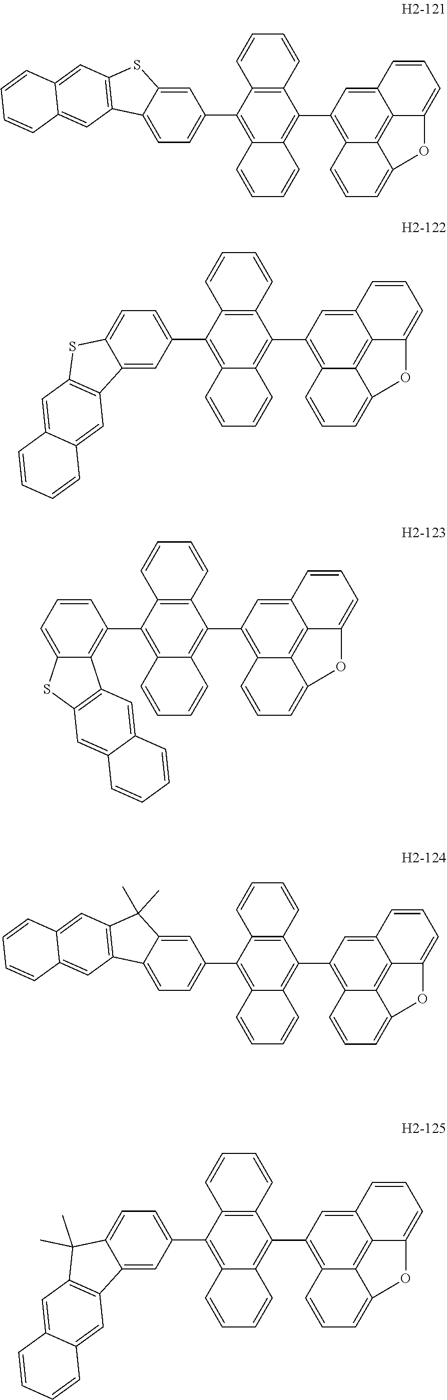 [SMILES: c1ccc2cc3c(cc2c1)sc1cc(-c2c4ccccc4c(-c4cc5cccc6oc7cccc4c7c56)c4ccccc24)ccc13 ; c1ccc2cc3c(cc2c1)sc1ccc(-c2c4ccccc4c(-c4cc5cccc6oc7cccc4c7c56)c4ccccc24)cc13 ; c1ccc2cc3c(cc2c1)sc1cccc(-c2c4ccccc4c(-c4cc5cccc6oc7cccc4c7c56)c4ccccc24)c13 ; CC1(C)c2cc(-c3c4ccccc4c(-c4cc5cccc6oc7cccc4c7c56)c4ccccc34)ccc2-c2cc3ccccc3cc21 ; CC1(C)c2ccc(-c3c4ccccc4c(-c4cc5cccc6oc7cccc4c7c56)c4ccccc34)cc2-c2cc3ccccc3cc21]

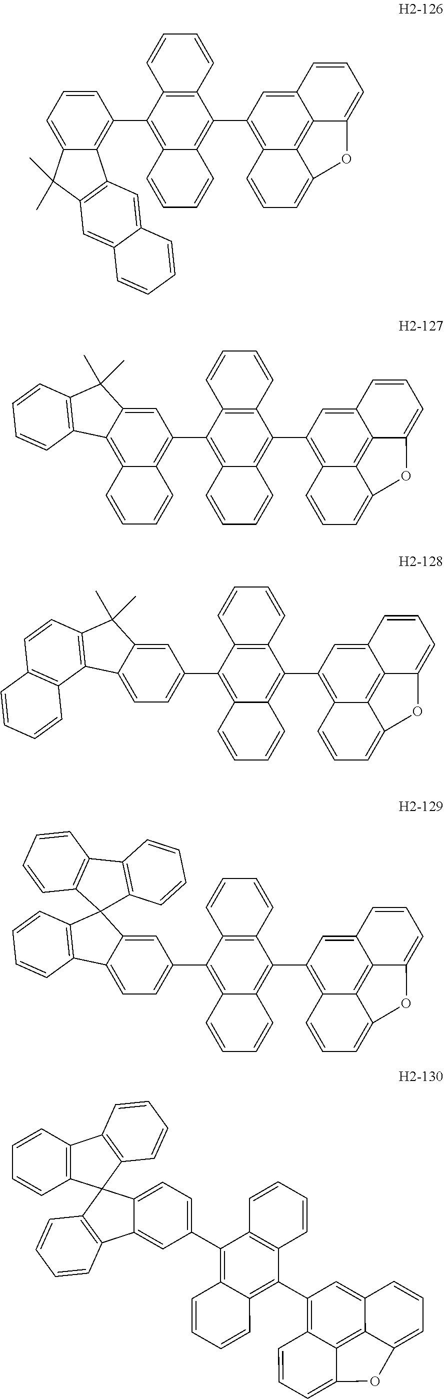 [SMILES: CC1(C)c2cc3ccccc3cc2-c2c(-c3c4ccccc4c(-c4cc5cccc6oc7cccc4c7c56)c4ccccc34)cccc21 ; CC1(C)c2ccccc2-c2c1cc(-c1c3ccccc3c(-c3cc4cccc5oc6cccc3c6c45)c3ccccc13)c1ccccc21 ; CC1(C)c2cc(-c3c4ccccc4c(-c4cc5cccc6oc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6oc7cccc4c7c56)c4ccccc34)cc21 ; c1ccc2c(c1)-c1ccccc1C21c2ccccc2-c2cc(-c3c4ccccc4c(-c4cc5cccc6oc7cccc4c7c56)c4ccccc34)ccc21]

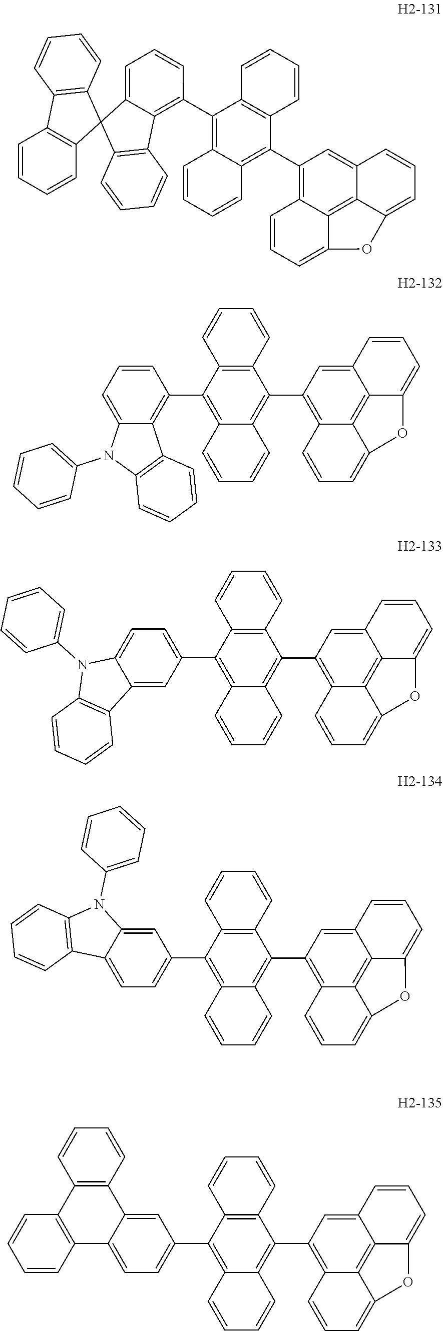 [SMILES: c1ccc2c(c1)-c1ccccc1C21c2ccccc2-c2c(-c3c4ccccc4c(-c4cc5cccc6oc7cccc4c7c56)c4ccccc34)cccc21 ; c1ccc(-n2c3ccccc3c3c(-c4c5ccccc5c(-c5cc6cccc7oc8cccc5c8c67)c5ccccc45)cccc32)cc1 ; c1ccc(-n2c3ccccc3c3cc(-c4c5ccccc5c(-c5cc6cccc7oc8cccc5c8c67)c5ccccc45)ccc32)cc1 ; c1ccc(-n2c3ccccc3c3ccc(-c4c5ccccc5c(-c5cc6cccc7oc8cccc5c8c67)c5ccccc45)cc32)cc1 ; c1cc2cc(-c3c4ccccc4c(-c4ccc5c6ccccc6c6ccccc6c5c4)c4ccccc34)c3cccc4oc(c1)c2c43]

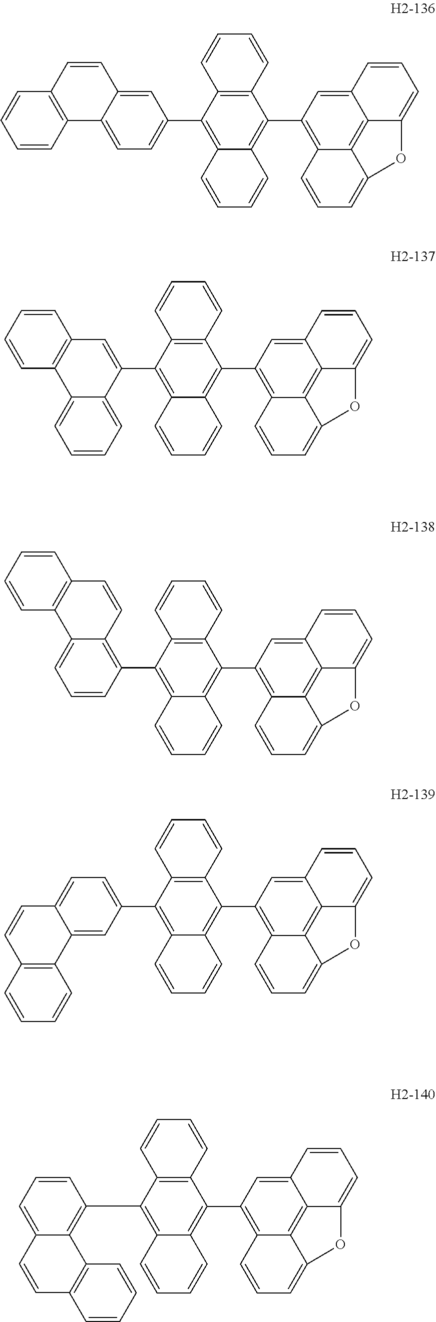 [SMILES: c1ccc2c(c1)ccc1cc(-c3c4ccccc4c(-c4cc5cccc6oc7cccc4c7c56)c4ccccc34)ccc12 ; c1ccc2c(c1)cc(-c1c3ccccc3c(-c3cc4cccc5oc6cccc3c6c45)c3ccccc13)c1ccccc12 ; c1ccc2c(c1)ccc1c(-c3c4ccccc4c(-c4cc5cccc6oc7cccc4c7c56)c4ccccc34)cccc12 ; c1ccc2c(c1)ccc1ccc(-c3c4ccccc4c(-c4cc5cccc6oc7cccc4c7c56)c4ccccc34)cc12 ; c1ccc2c(c1)ccc1cccc(-c3c4ccccc4c(-c4cc5cccc6oc7cccc4c7c56)c4ccccc34)c12]

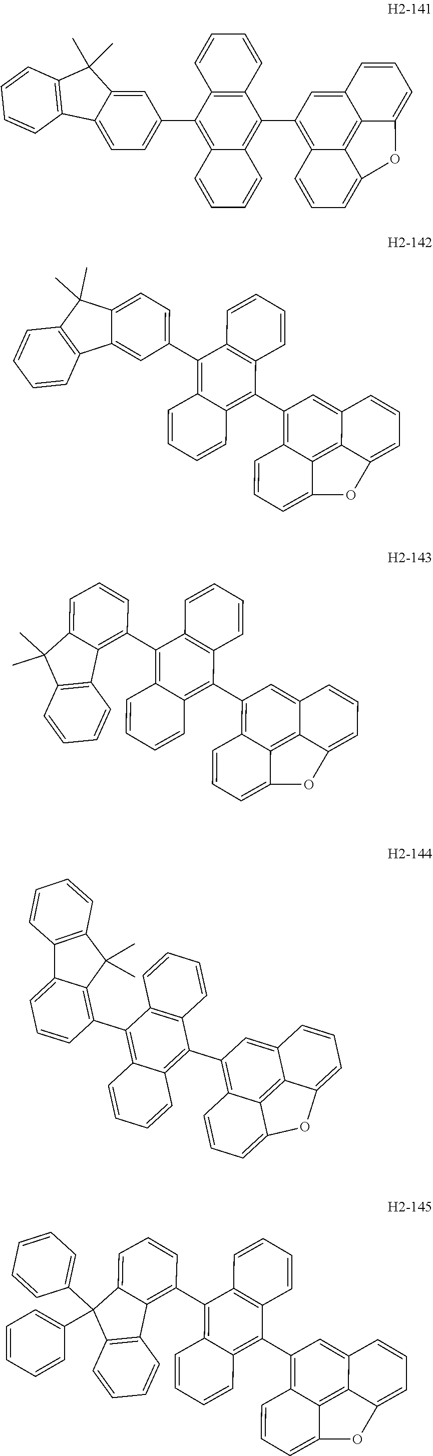 [SMILES: CC1(C)c2ccccc2-c2ccc(-c3c4ccccc4c(-c4cc5cccc6oc7cccc4c7c56)c4ccccc34)cc21 ; CC1(C)c2ccccc2-c2cc(-c3c4ccccc4c(-c4cc5cccc6oc7cccc4c7c56)c4ccccc34)ccc21 ; CC1(C)c2ccccc2-c2c(-c3c4ccccc4c(-c4cc5cccc6oc7cccc4c7c56)c4ccccc34)cccc21 ; CC1(C)c2ccccc2-c2cccc(-c3c4ccccc4c(-c4cc5cccc6oc7cccc4c7c56)c4ccccc34)c21 ; c1ccc(C2(c3ccccc3)c3ccccc3-c3c(-c4c5ccccc5c(-c5cc6cccc7oc8cccc5c8c67)c5ccccc45)cccc32)cc1]

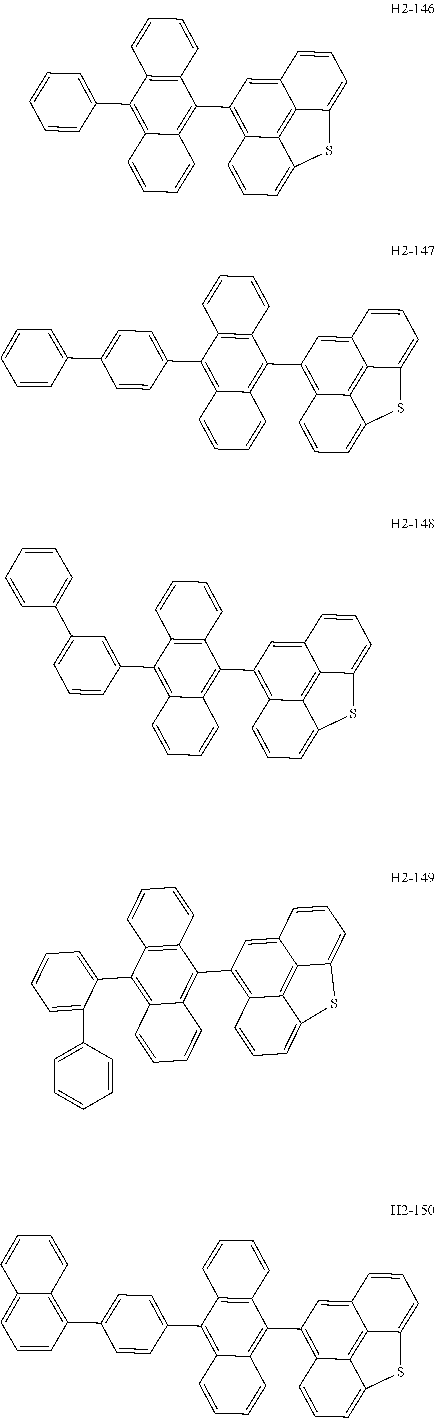 [SMILES: c1ccc(-c2c3ccccc3c(-c3cc4cccc5sc6cccc3c6c45)c3ccccc23)cc1 ; c1ccc(-c2ccc(-c3c4ccccc4c(-c4cc5cccc6sc7cccc4c7c56)c4ccccc34)cc2)cc1 ; c1ccc(-c2cccc(-c3c4ccccc4c(-c4cc5cccc6sc7cccc4c7c56)c4ccccc34)c2)cc1 ; c1ccc(-c2ccccc2-c2c3ccccc3c(-c3cc4cccc5sc6cccc3c6c45)c3ccccc23)cc1 ; c1ccc2c(-c3ccc(-c4c5ccccc5c(-c5cc6cccc7sc8cccc5c8c67)c5ccccc45)cc3)cccc2c1]

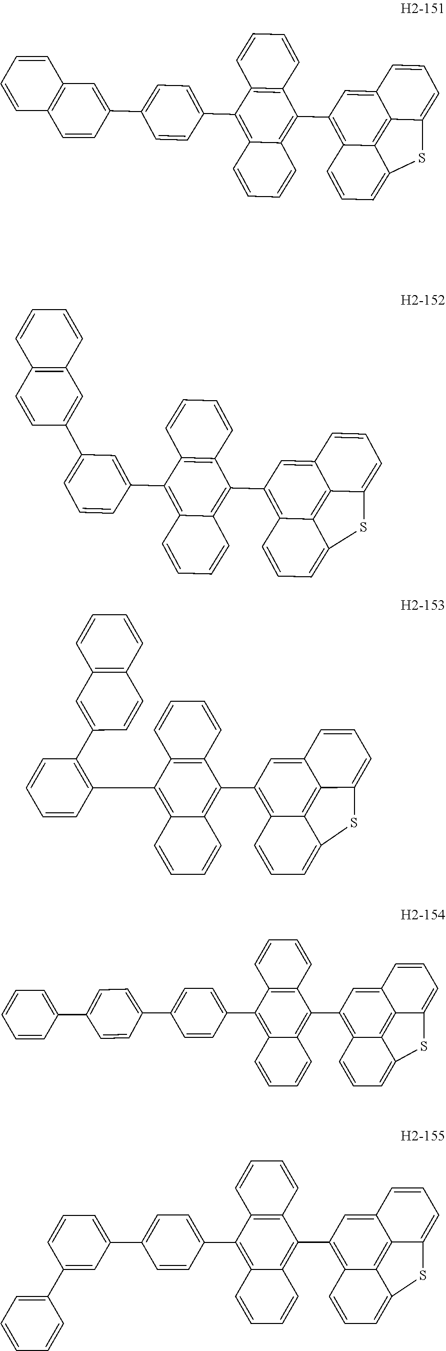 [SMILES: c1ccc2cc(-c3ccc(-c4c5ccccc5c(-c5cc6cccc7sc8cccc5c8c67)c5ccccc45)cc3)ccc2c1 ; c1cc(-c2ccc3ccccc3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3ccccc3c2)c1 ; c1ccc(-c2ccc(-c3ccc(-c4c5ccccc5c(-c5cc6cccc7sc8cccc5c8c67)c5ccccc45)cc3)cc2)cc1 ; c1ccc(-c2cccc(-c3ccc(-c4c5ccccc5c(-c5cc6cccc7sc8cccc5c8c67)c5ccccc45)cc3)c2)cc1]

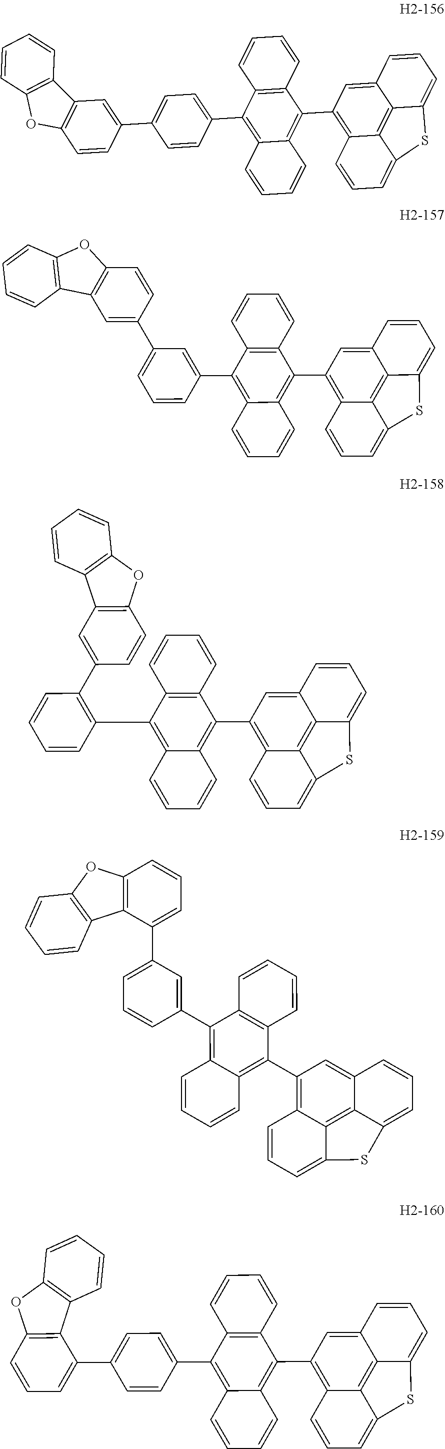 [SMILES: c1ccc2c(c1)oc1ccc(-c3ccc(-c4c5ccccc5c(-c5cc6cccc7sc8cccc5c8c67)c5ccccc45)cc3)cc12 ; c1cc(-c2ccc3oc4ccccc4c3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3oc4ccccc4c3c2)c1 ; c1cc(-c2c3ccccc3c(-c3cc4cccc5sc6cccc3c6c45)c3ccccc23)cc(-c2cccc3oc4ccccc4c23)c1 ; c1ccc2c(c1)oc1cccc(-c3ccc(-c4c5ccccc5c(-c5cc6cccc7sc8cccc5c8c67)c5ccccc45)cc3)c12]

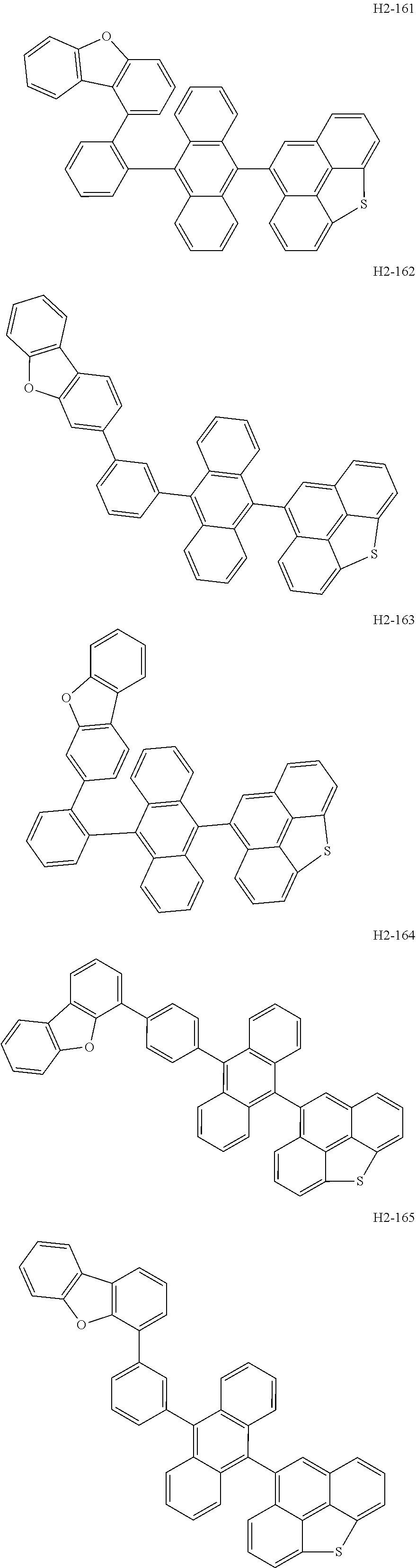 [SMILES: c1ccc(-c2cccc3oc4ccccc4c23)c(-c2c3ccccc3c(-c3cc4cccc5sc6cccc3c6c45)c3ccccc23)c1 ; c1cc(-c2ccc3c(c2)oc2ccccc23)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3c(c2)oc2ccccc23)c1 ; c1ccc2c(c1)oc1c(-c3ccc(-c4c5ccccc5c(-c5cc6cccc7sc8cccc5c8c67)c5ccccc45)cc3)cccc12 ; c1cc(-c2c3ccccc3c(-c3cc4cccc5sc6cccc3c6c45)c3ccccc23)cc(-c2cccc3c2oc2ccccc23)c1]

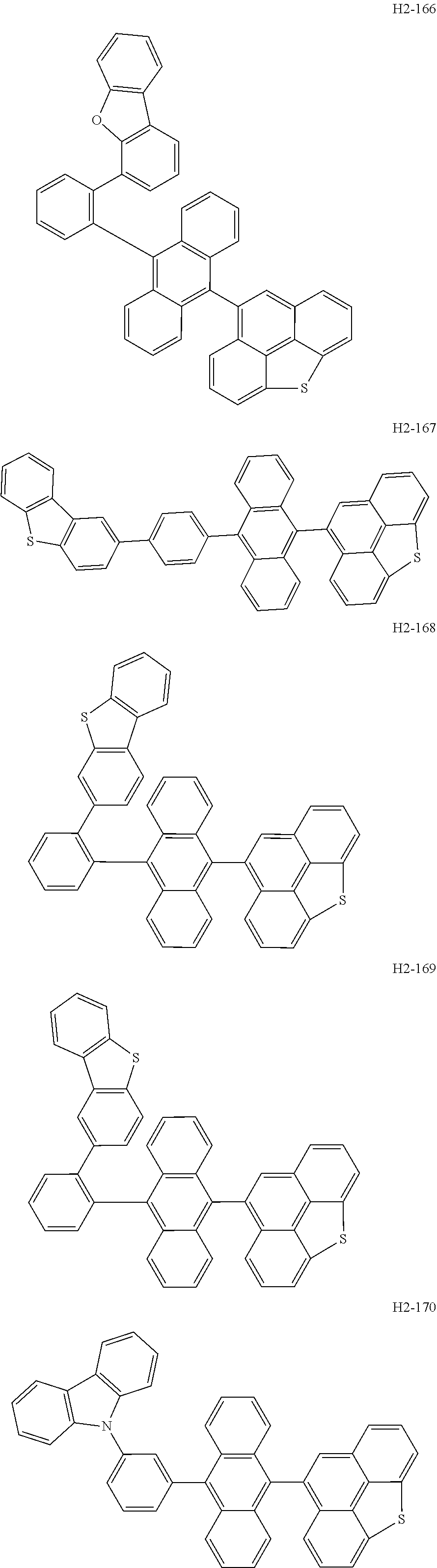 [SMILES: c1ccc(-c2cccc3c2oc2ccccc23)c(-c2c3ccccc3c(-c3cc4cccc5sc6cccc3c6c45)c3ccccc23)c1 ; c1ccc2c(c1)sc1ccc(-c3ccc(-c4c5ccccc5c(-c5cc6cccc7sc8cccc5c8c67)c5ccccc45)cc3)cc12 ; c1ccc(-c2c3ccccc3c(-c3cc4cccc5sc6cccc3c6c45)c3ccccc23)c(-c2ccc3c(c2)sc2ccccc23)c1 ; c1ccc(-c2c3ccccc3c(-c3cc4cccc5sc6cccc3c6c45)c3ccccc23)c(-c2ccc3sc4ccccc4c3c2)c1 ; c1cc(-c2c3ccccc3c(-c3cc4cccc5sc6cccc3c6c45)c3ccccc23)cc(-n2c3ccccc3c3ccccc32)c1]

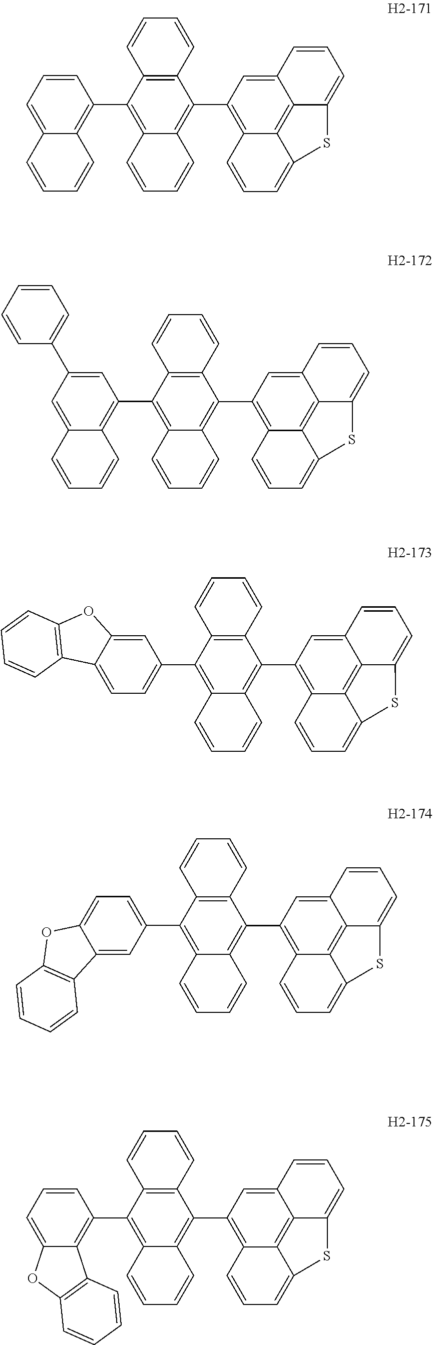 [SMILES: c1ccc2c(-c3c4ccccc4c(-c4cc5cccc6sc7cccc4c7c56)c4ccccc34)cccc2c1 ; c1ccc(-c2cc(-c3c4ccccc4c(-c4cc5cccc6sc7cccc4c7c56)c4ccccc34)c3ccccc3c2)cc1 ; c1ccc2c(c1)oc1cc(-c3c4ccccc4c(-c4cc5cccc6sc7cccc4c7c56)c4ccccc34)ccc12 ; c1ccc2c(c1)oc1ccc(-c3c4ccccc4c(-c4cc5cccc6sc7cccc4c7c56)c4ccccc34)cc12 ; c1ccc2c(c1)oc1cccc(-c3c4ccccc4c(-c4cc5cccc6sc7cccc4c7c56)c4ccccc34)c12]

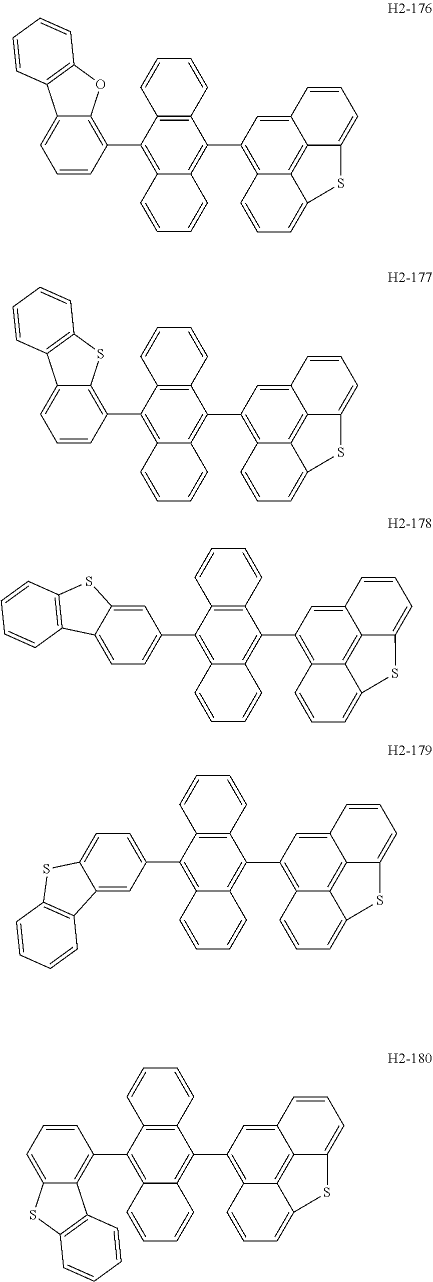 [SMILES: c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12]

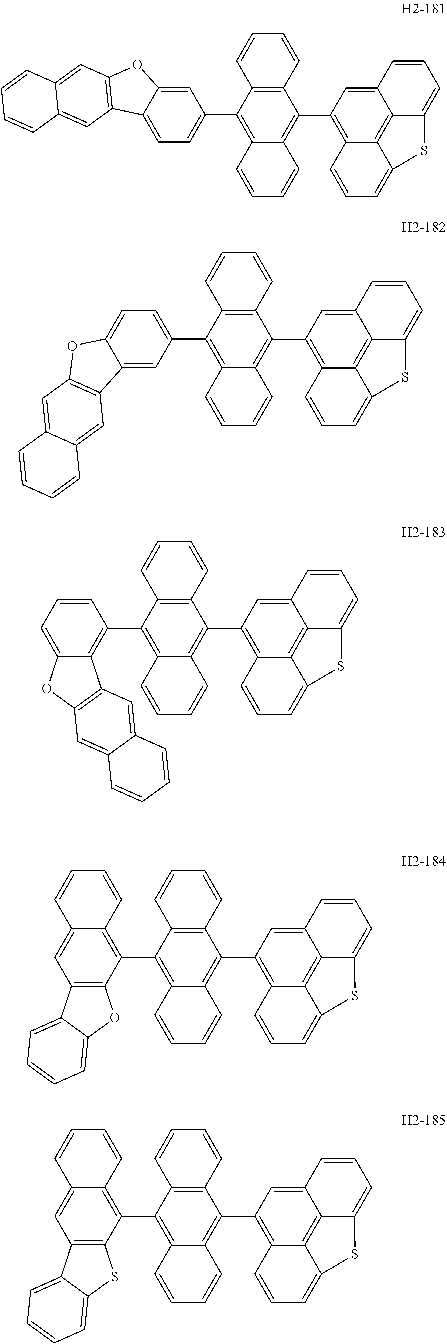 [SMILES: c1ccc2cc3c(cc2c1)oc1cc(-c2c4ccccc4c(-c4cc5cccc6sc7cccc4c7c56)c4ccccc24)ccc13 ; c1ccc2cc3c(cc2c1)oc1ccc(-c2c4ccccc4c(-c4cc5cccc6sc7cccc4c7c56)c4ccccc24)cc13 ; c1ccc2cc3c(cc2c1)oc1cccc(-c2c4ccccc4c(-c4cc5cccc6sc7cccc4c7c56)c4ccccc24)c13 ; c1ccc2c(-c3c4ccccc4c(-c4cc5cccc6sc7cccc4c7c56)c4ccccc34)c3oc4ccccc4c3cc2c1 ; c1ccc2c(-c3c4ccccc4c(-c4cc5cccc6sc7cccc4c7c56)c4ccccc34)c3sc4ccccc4c3cc2c1]

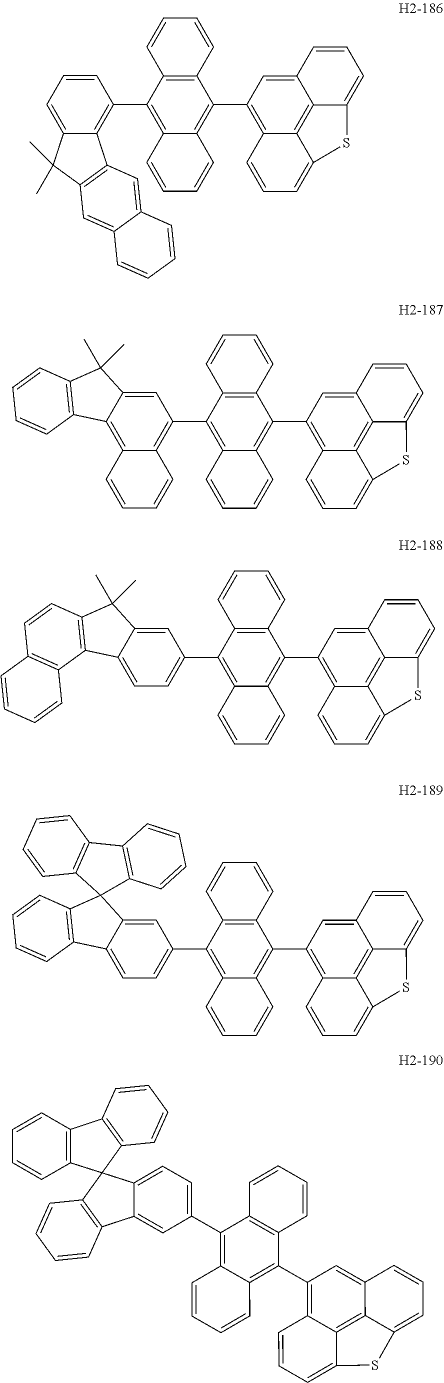 [SMILES: CC1(C)c2cc3ccccc3cc2-c2c(-c3c4ccccc4c(-c4cc5cccc6sc7cccc4c7c56)c4ccccc34)cccc21 ; CC1(C)c2ccccc2-c2c1cc(-c1c3ccccc3c(-c3cc4cccc5sc6cccc3c6c45)c3ccccc13)c1ccccc21 ; CC1(C)c2cc(-c3c4ccccc4c(-c4cc5cccc6sc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6sc7cccc4c7c56)c4ccccc34)cc21 ; c1ccc2c(c1)-c1ccccc1C21c2ccccc2-c2cc(-c3c4ccccc4c(-c4cc5cccc6sc7cccc4c7c56)c4ccccc34)ccc21]

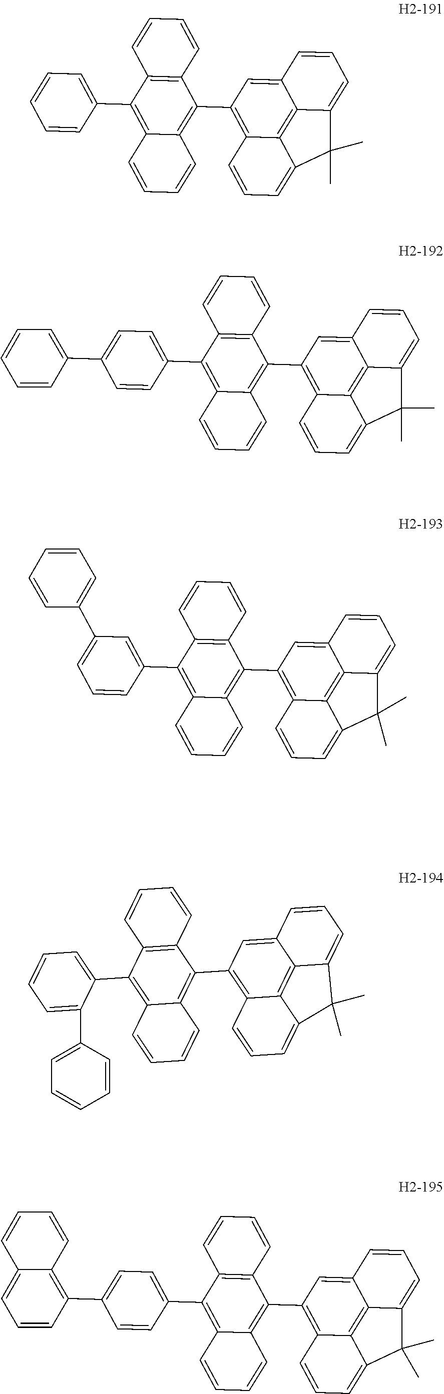

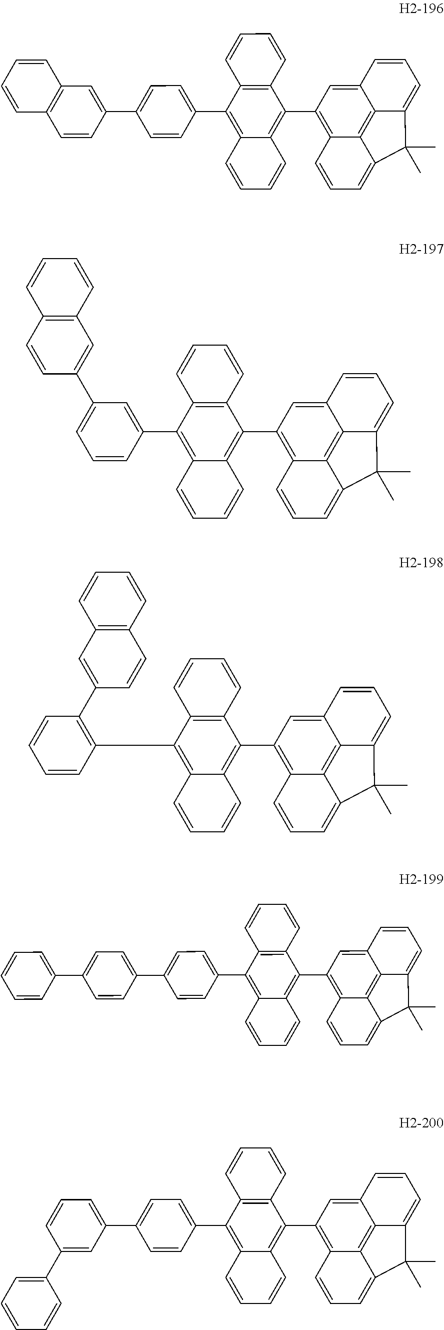

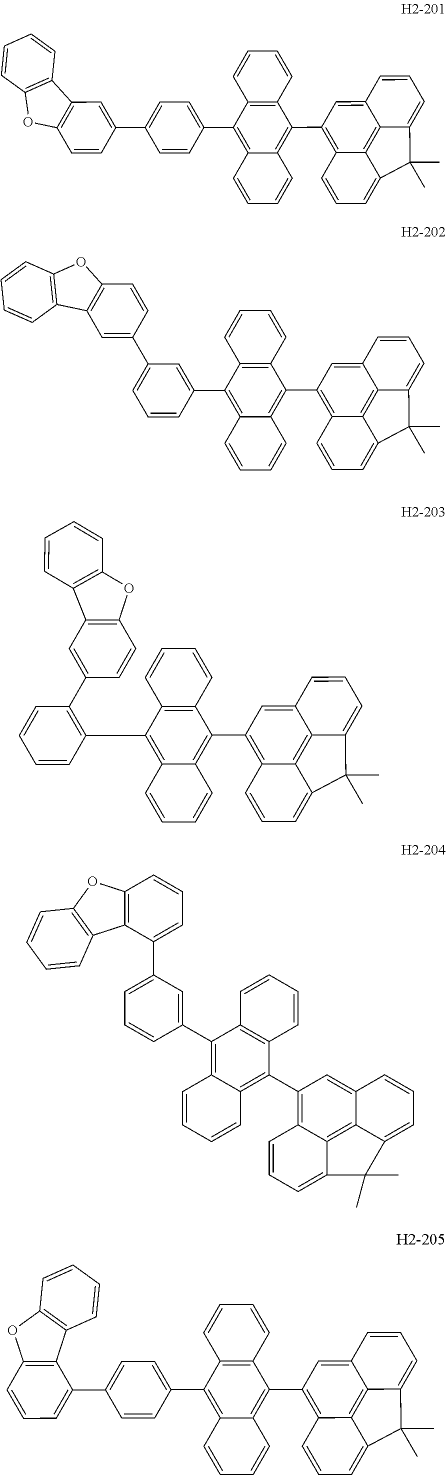 [SMILES: CC1(C)c2cccc3cc(-c4c5ccccc5c(-c5ccc(-c6ccc7oc8ccccc8c7c6)cc5)c5ccccc45)c4cccc1c4c23 ; CC1(C)c2cccc3cc(-c4c5ccccc5c(-c5cccc(-c6ccc7oc8ccccc8c7c6)c5)c5ccccc45)c4cccc1c4c23 ; CC1(C)c2cccc3cc(-c4c5ccccc5c(-c5ccccc5-c5ccc6oc7ccccc7c6c5)c5ccccc45)c4cccc1c4c23 ; CC1(C)c2cccc3cc(-c4c5ccccc5c(-c5cccc(-c6cccc7oc8ccccc8c67)c5)c5ccccc45)c4cccc1c4c23 ; CC1(C)c2cccc3cc(-c4c5ccccc5c(-c5ccc(-c6cccc7oc8ccccc8c67)cc5)c5ccccc45)c4cccc1c4c23]

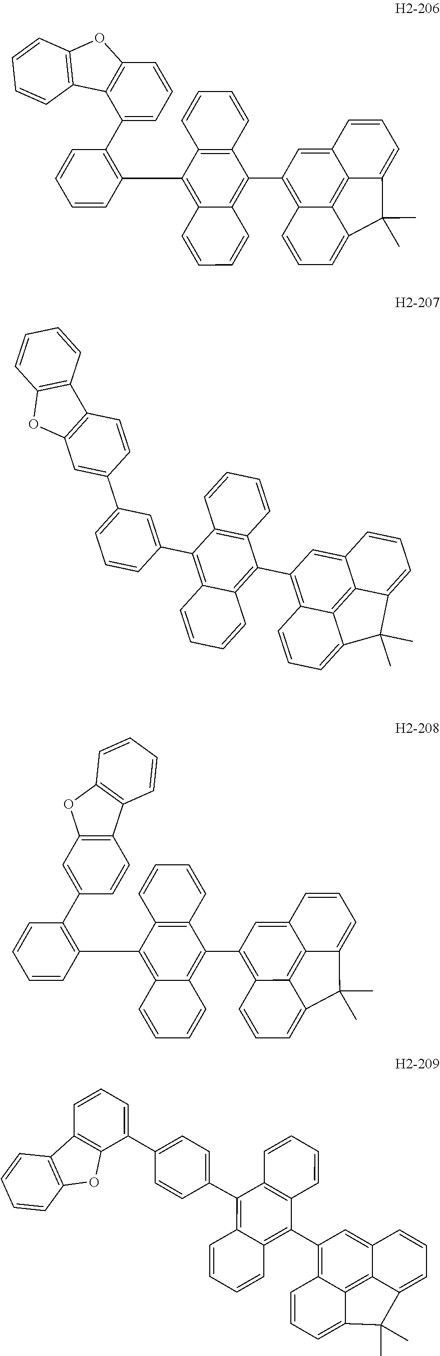 [SMILES: CC1(C)c2cccc3cc(-c4c5ccccc5c(-c5ccccc5-c5cccc6oc7ccccc7c56)c5ccccc45)c4cccc1c4c23 ; CC1(C)c2cccc3cc(-c4c5ccccc5c(-c5cccc(-c6ccc7c(c6)oc6ccccc67)c5)c5ccccc45)c4cccc1c4c23 ; CC1(C)c2cccc3cc(-c4c5ccccc5c(-c5ccccc5-c5ccc6c(c5)oc5ccccc56)c5ccccc45)c4cccc1c4c23 ; CC1(C)c2cccc3cc(-c4c5ccccc5c(-c5ccc(-c6cccc7c6oc6ccccc67)cc5)c5ccccc45)c4cccc1c4c23]

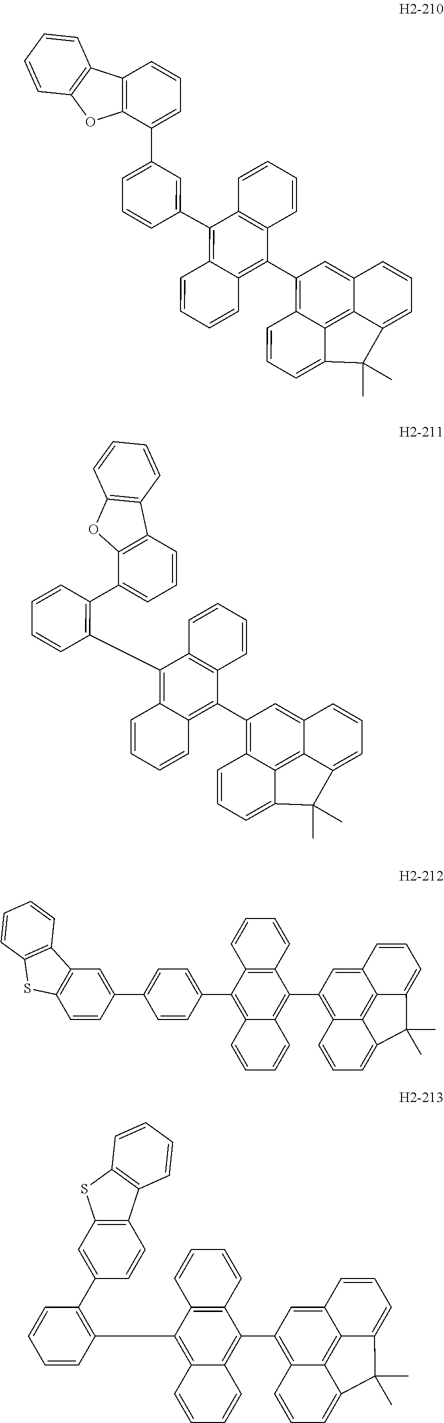 [SMILES: CC1(C)c2cccc3cc(-c4c5ccccc5c(-c5cccc(-c6cccc7c6oc6ccccc67)c5)c5ccccc45)c4cccc1c4c23 ; CC1(C)c2cccc3cc(-c4c5ccccc5c(-c5ccccc5-c5cccc6c5oc5ccccc56)c5ccccc45)c4cccc1c4c23 ; CC1(C)c2cccc3cc(-c4c5ccccc5c(-c5ccc(-c6ccc7sc8ccccc8c7c6)cc5)c5ccccc45)c4cccc1c4c23 ; CC1(C)c2cccc3cc(-c4c5ccccc5c(-c5ccccc5-c5ccc6c(c5)sc5ccccc56)c5ccccc45)c4cccc1c4c23]

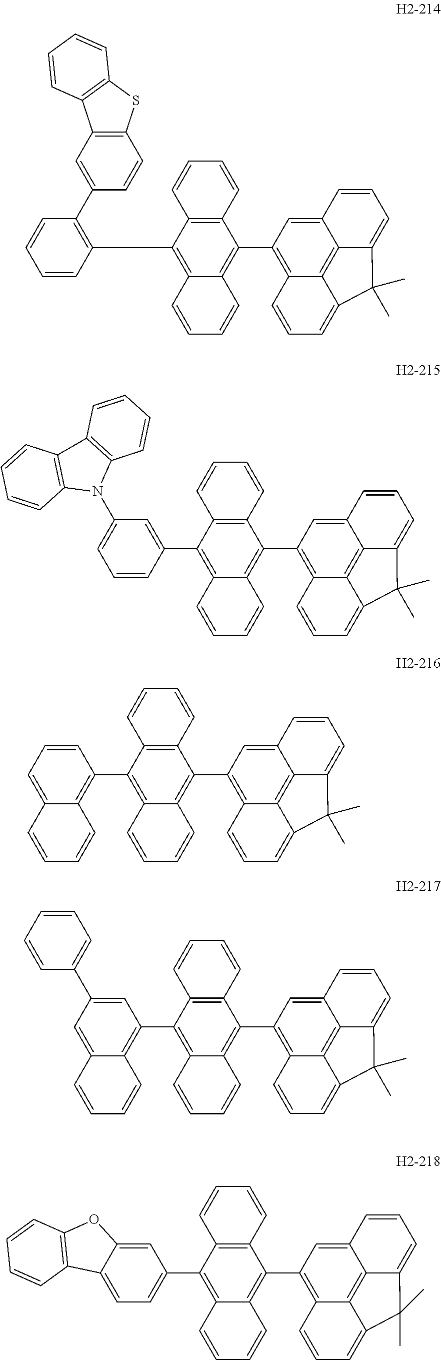

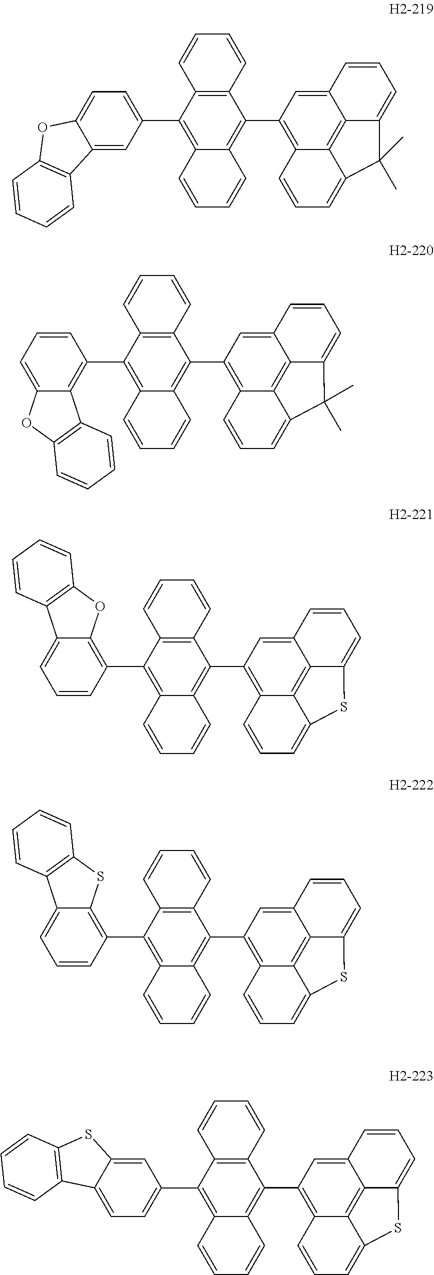 [SMILES: CC1(C)c2cccc3cc(-c4c5ccccc5c(-c5ccc6oc7ccccc7c6c5)c5ccccc45)c4cccc1c4c23 ; CC1(C)c2cccc3cc(-c4c5ccccc5c(-c5cccc6oc7ccccc7c56)c5ccccc45)c4cccc1c4c23 ; c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12]

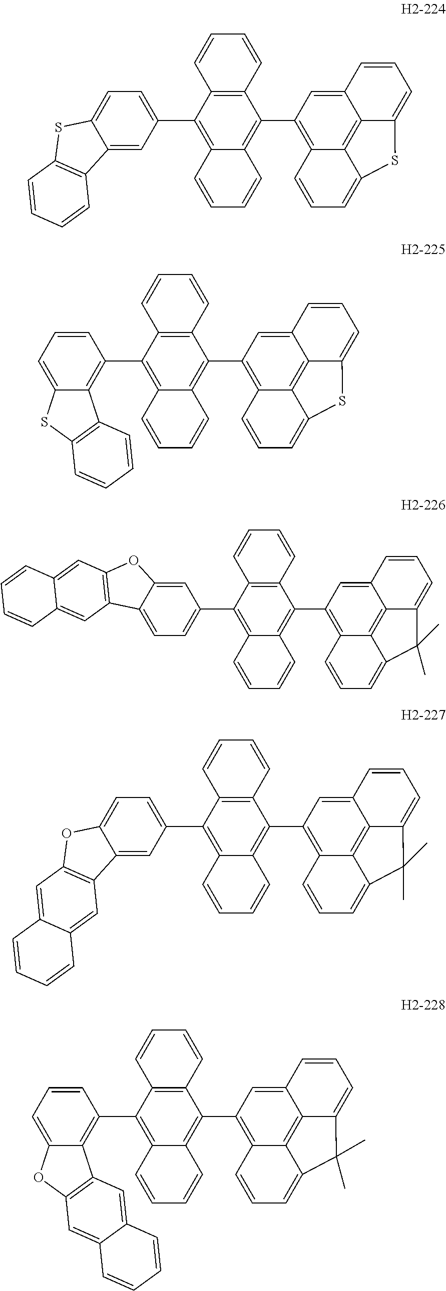 [SMILES: c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12 ; CC1(C)c2cccc3cc(-c4c5ccccc5c(-c5ccc6c(c5)oc5cc7ccccc7cc56)c5ccccc45)c4cccc1c4c23 ; CC1(C)c2cccc3cc(-c4c5ccccc5c(-c5ccc6oc7cc8ccccc8cc7c6c5)c5ccccc45)c4cccc1c4c23 ; CC1(C)c2cccc3cc(-c4c5ccccc5c(-c5cccc6oc7cc8ccccc8cc7c56)c5ccccc45)c4cccc1c4c23]

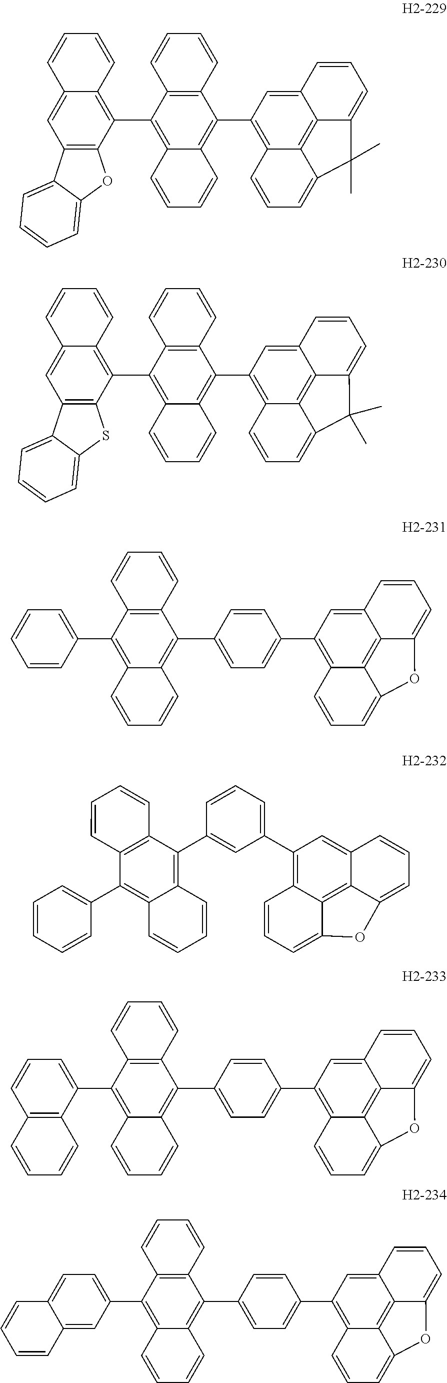

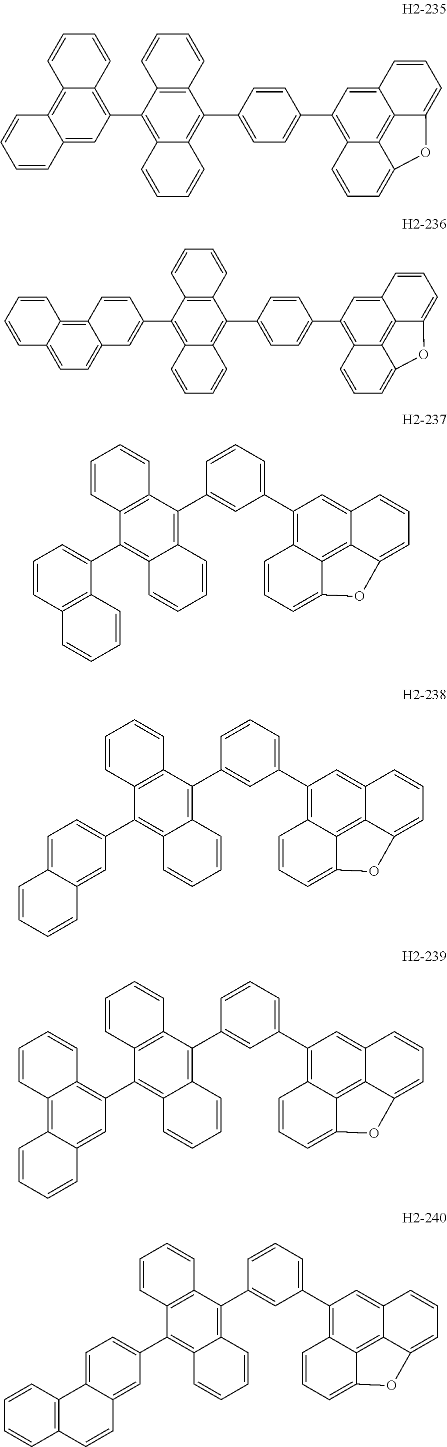 [SMILES: c1ccc2c(c1)cc(-c1c3ccccc3c(-c3ccc(-c4cc5cccc6oc7cccc4c7c56)cc3)c3ccccc13)c1ccccc12 ; c1ccc2c(c1)ccc1cc(-c3c4ccccc4c(-c4ccc(-c5cc6cccc7oc8cccc5c8c67)cc4)c4ccccc34)ccc12 ; c1cc(-c2c3ccccc3c(-c3cccc4ccccc34)c3ccccc23)cc(-c2cc3cccc4oc5cccc2c5c34)c1 ; c1cc(-c2c3ccccc3c(-c3ccc4ccccc4c3)c3ccccc23)cc(-c2cc3cccc4oc5cccc2c5c34)c1 ; c1cc(-c2c3ccccc3c(-c3cc4ccccc4c4ccccc34)c3ccccc23)cc(-c2cc3cccc4oc5cccc2c5c34)c1 ; c1cc(-c2c3ccccc3c(-c3ccc4c(ccc5ccccc54)c3)c3ccccc23)cc(-c2cc3cccc4oc5cccc2c5c34)c1]

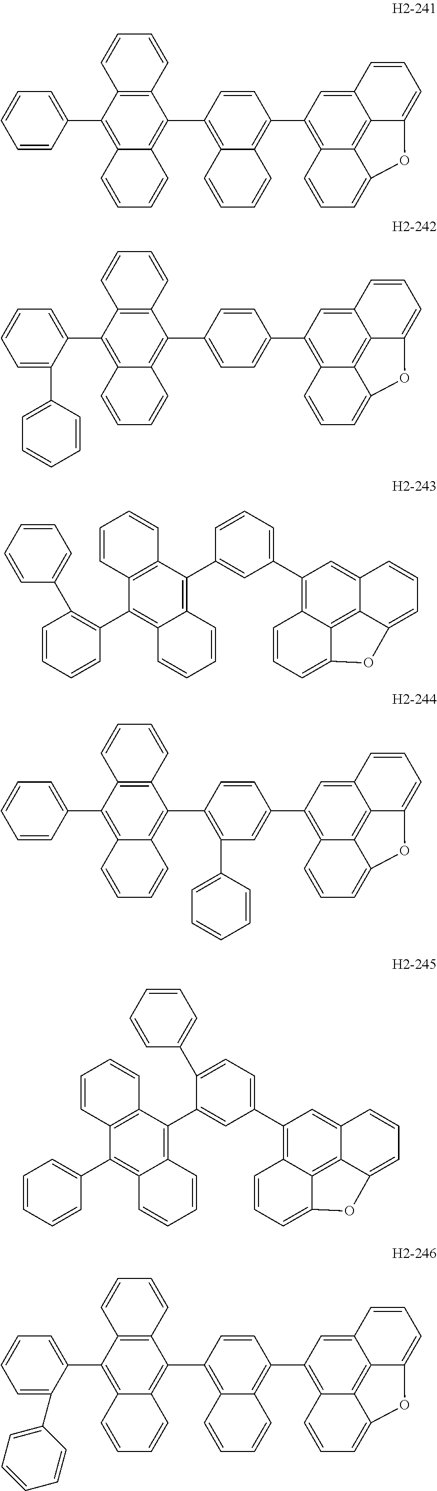

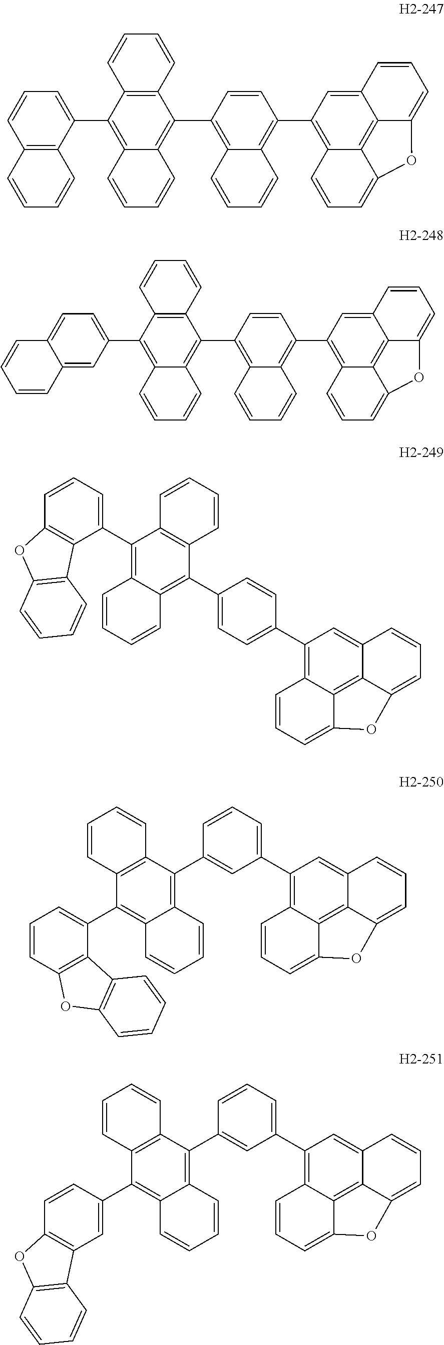 [SMILES: c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1 ; c1ccc2c(c1)oc1cccc(-c3c4ccccc4c(-c4ccc(-c5cc6cccc7oc8cccc5c8c67)cc4)c4ccccc34)c12 ; c1cc(-c2c3ccccc3c(-c3cccc4oc5ccccc5c34)c3ccccc23)cc(-c2cc3cccc4oc5cccc2c5c34)c1 ; c1cc(-c2c3ccccc3c(-c3ccc4oc5ccccc5c4c3)c3ccccc23)cc(-c2cc3cccc4oc5cccc2c5c34)c1]

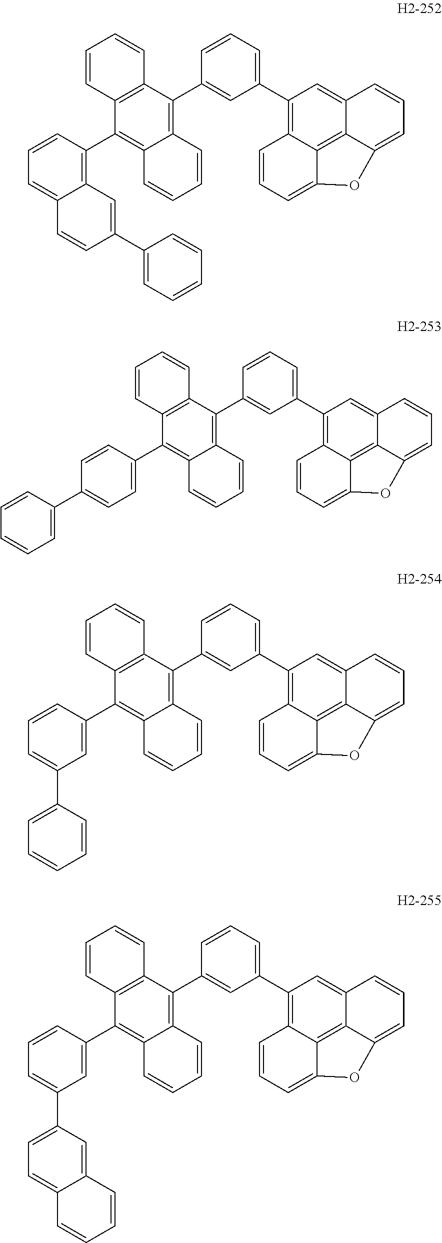 [SMILES: c1ccc(-c2ccc3cccc(-c4c5ccccc5c(-c5cccc(-c6cc7cccc8oc9cccc6c9c78)c5)c5ccccc45)c3c2)cc1 ; c1ccc(-c2ccc(-c3c4ccccc4c(-c4cccc(-c5cc6cccc7oc8cccc5c8c67)c4)c4ccccc34)cc2)cc1 ; c1ccc(-c2cccc(-c3c4ccccc4c(-c4cccc(-c5cc6cccc7oc8cccc5c8c67)c4)c4ccccc34)c2)cc1 ; c1cc(-c2ccc3ccccc3c2)cc(-c2c3ccccc3c(-c3cccc(-c4cc5cccc6oc7cccc4c7c56)c3)c3ccccc23)c1]

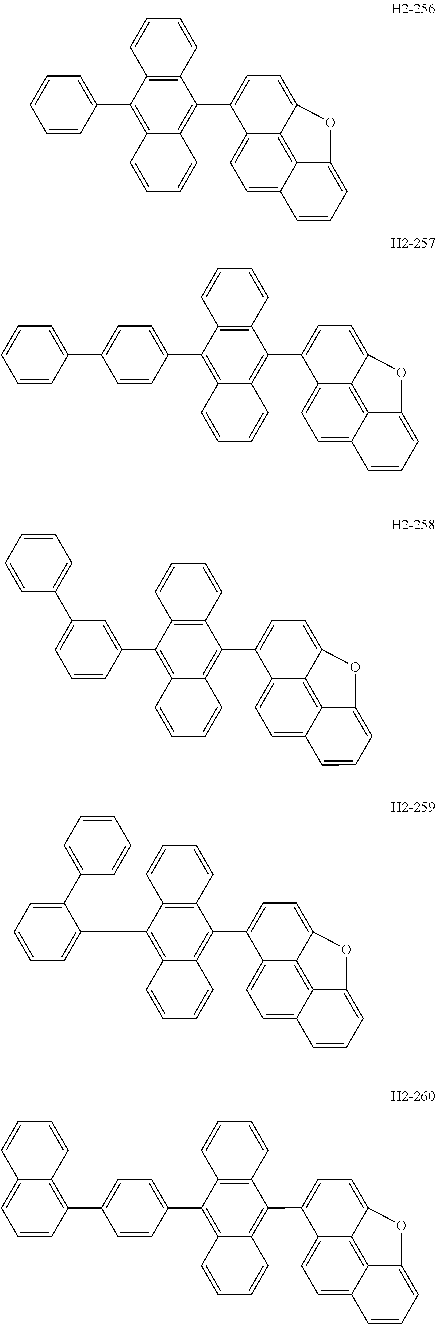 [SMILES: c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1 ; c1ccc(-c2ccccc2-c2c3ccccc3c(-c3ccc4oc5cccc6ccc3c4c65)c3ccccc23)cc1 ; c1ccc2c(-c3ccc(-c4c5ccccc5c(-c5ccc6oc7cccc8ccc5c6c87)c5ccccc45)cc3)cccc2c1]

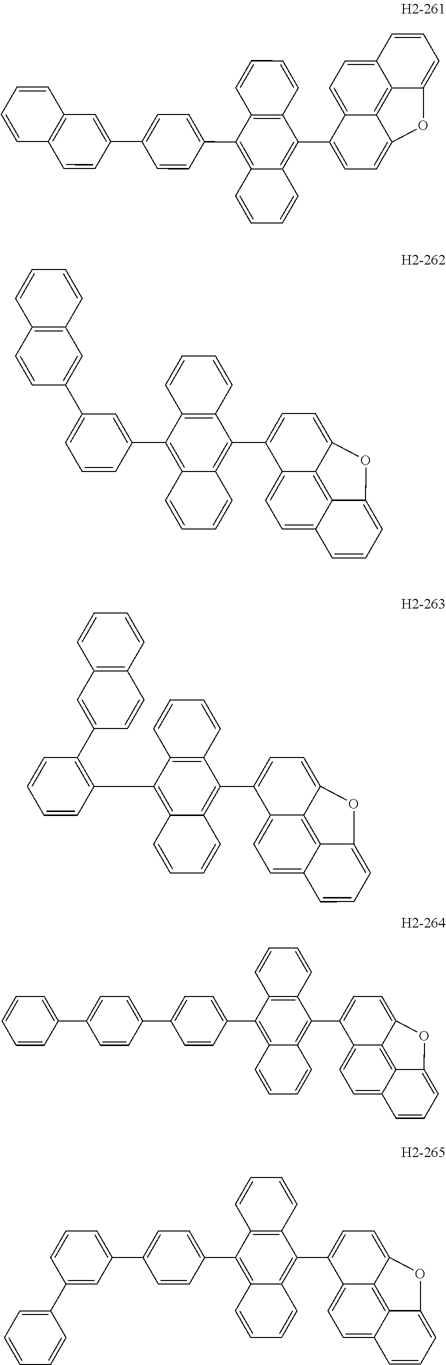 [SMILES: c1ccc2cc(-c3ccc(-c4c5ccccc5c(-c5ccc6oc7cccc8ccc5c6c87)c5ccccc45)cc3)ccc2c1 ; c1cc(-c2ccc3ccccc3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3ccccc3c2)c1 ; c1ccc(-c2ccc(-c3ccc(-c4c5ccccc5c(-c5ccc6oc7cccc8ccc5c6c87)c5ccccc45)cc3)cc2)cc1 ; c1ccc(-c2cccc(-c3ccc(-c4c5ccccc5c(-c5ccc6oc7cccc8ccc5c6c87)c5ccccc45)cc3)c2)cc1]

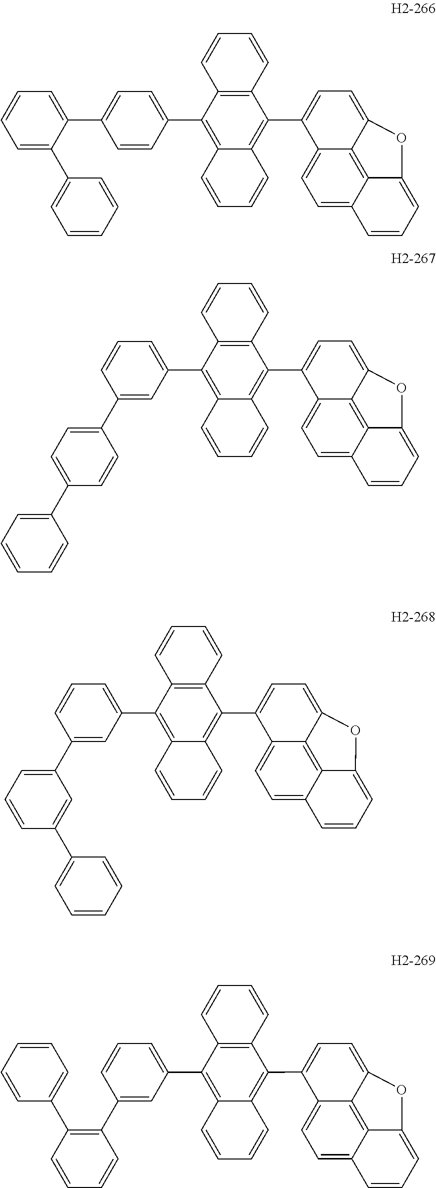 [SMILES: c1ccc(-c2ccccc2-c2ccc(-c3c4ccccc4c(-c4ccc5oc6cccc7ccc4c5c76)c4ccccc34)cc2)cc1 ; c1ccc(-c2ccc(-c3cccc(-c4c5ccccc5c(-c5ccc6oc7cccc8ccc5c6c87)c5ccccc45)c3)cc2)cc1 ; c1ccc(-c2cccc(-c3cccc(-c4c5ccccc5c(-c5ccc6oc7cccc8ccc5c6c87)c5ccccc45)c3)c2)cc1 ; c1ccc(-c2ccccc2-c2cccc(-c3c4ccccc4c(-c4ccc5oc6cccc7ccc4c5c76)c4ccccc34)c2)cc1]

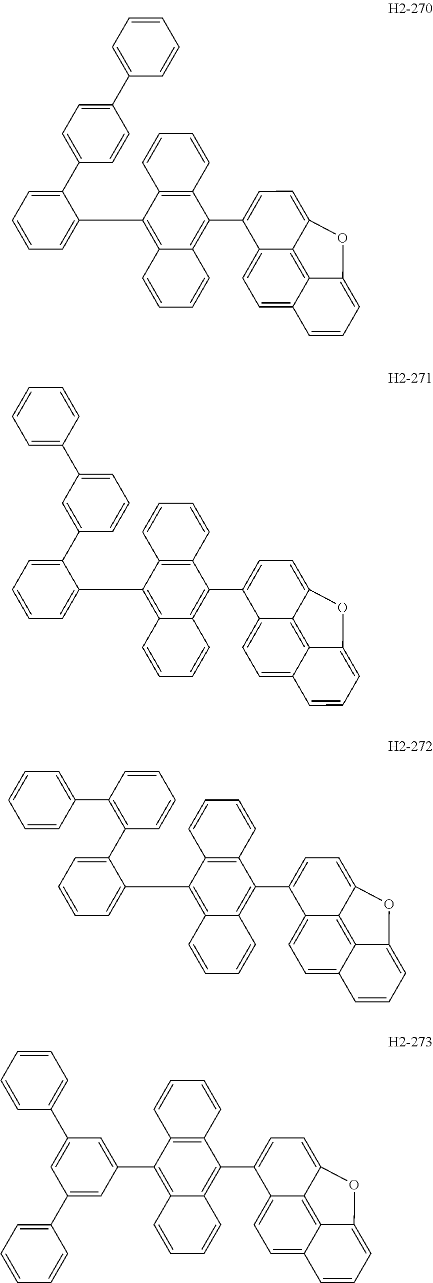 [SMILES: c1ccc(-c2ccc(-c3ccccc3-c3c4ccccc4c(-c4ccc5oc6cccc7ccc4c5c76)c4ccccc34)cc2)cc1 ; c1ccc(-c2cccc(-c3ccccc3-c3c4ccccc4c(-c4ccc5oc6cccc7ccc4c5c76)c4ccccc34)c2)cc1 ; c1ccc(-c2ccccc2-c2ccccc2-c2c3ccccc3c(-c3ccc4oc5cccc6ccc3c4c65)c3ccccc23)cc1 ; c1ccc(-c2cc(-c3ccccc3)cc(-c3c4ccccc4c(-c4ccc5oc6cccc7ccc4c5c76)c4ccccc34)c2)cc1]

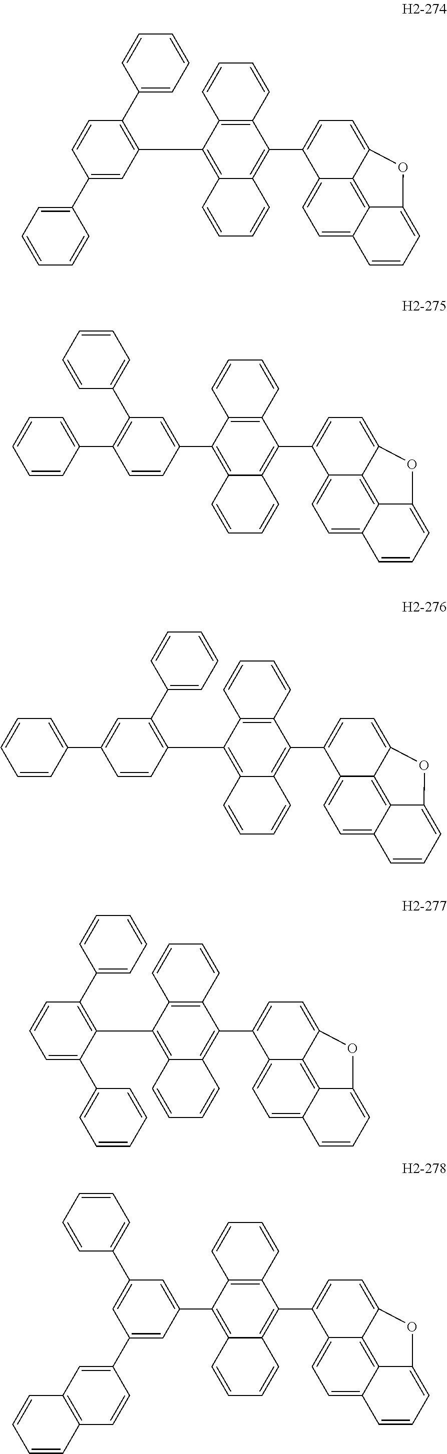 [SMILES: c1ccc(-c2ccc(-c3ccccc3)c(-c3c4ccccc4c(-c4ccc5oc6cccc7ccc4c5c76)c4ccccc34)c2)cc1 ; c1ccc(-c2ccc(-c3c4ccccc4c(-c4ccc5oc6cccc7ccc4c5c76)c4ccccc34)cc2-c2ccccc2)cc1 ; c1ccc(-c2ccc(-c3c4ccccc4c(-c4ccc5oc6cccc7ccc4c5c76)c4ccccc34)c(-c3ccccc3)c2)cc1 ; c1ccc(-c2cccc(-c3ccccc3)c2-c2c3ccccc3c(-c3ccc4oc5cccc6ccc3c4c65)c3ccccc23)cc1 ; c1ccc(-c2cc(-c3ccc4ccccc4c3)cc(-c3c4ccccc4c(-c4ccc5oc6cccc7ccc4c5c76)c4ccccc34)c2)cc1]

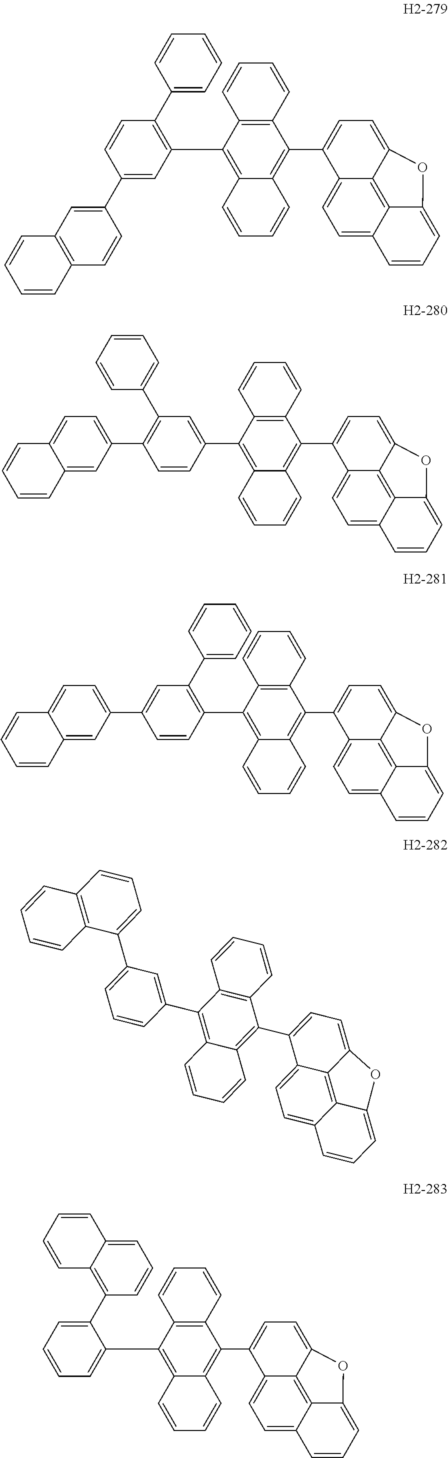 [SMILES: c1ccc(-c2ccc(-c3ccc4ccccc4c3)cc2-c2c3ccccc3c(-c3ccc4oc5cccc6ccc3c4c65)c3ccccc23)cc1 ; c1ccc(-c2cc(-c3c4ccccc4c(-c4ccc5oc6cccc7ccc4c5c76)c4ccccc34)ccc2-c2ccc3ccccc3c2)cc1 ; c1ccc(-c2cc(-c3ccc4ccccc4c3)ccc2-c2c3ccccc3c(-c3ccc4oc5cccc6ccc3c4c65)c3ccccc23)cc1 ; c1cc(-c2cccc3ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2cccc3ccccc23)c1]

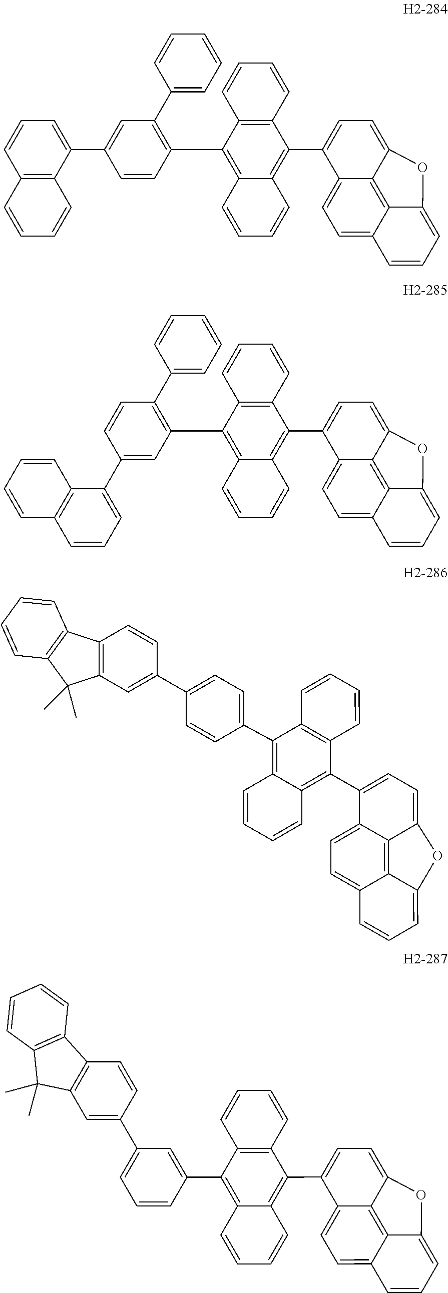 [SMILES: c1ccc(-c2cc(-c3cccc4ccccc34)ccc2-c2c3ccccc3c(-c3ccc4oc5cccc6ccc3c4c65)c3ccccc23)cc1 ; c1ccc(-c2ccc(-c3cccc4ccccc34)cc2-c2c3ccccc3c(-c3ccc4oc5cccc6ccc3c4c65)c3ccccc23)cc1 ; CC1(C)c2ccccc2-c2ccc(-c3ccc(-c4c5ccccc5c(-c5ccc6oc7cccc8ccc5c6c87)c5ccccc45)cc3)cc21 ; CC1(C)c2ccccc2-c2ccc(-c3cccc(-c4c5ccccc5c(-c5ccc6oc7cccc8ccc5c6c87)c5ccccc45)c3)cc21]

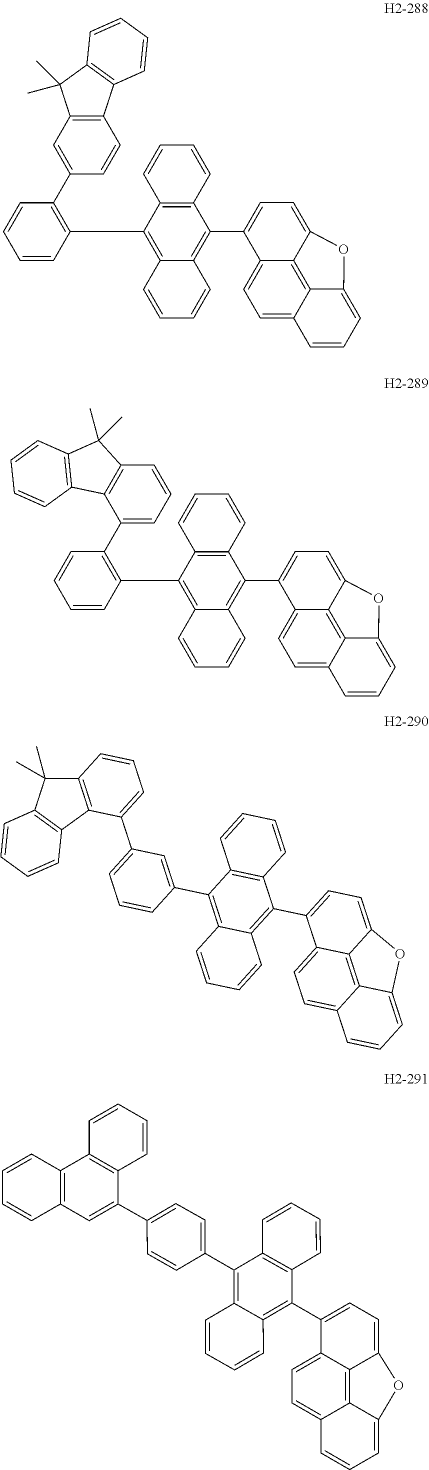 [SMILES: CC1(C)c2ccccc2-c2ccc(-c3ccccc3-c3c4ccccc4c(-c4ccc5oc6cccc7ccc4c5c76)c4ccccc34)cc21 ; CC1(C)c2ccccc2-c2c(-c3ccccc3-c3c4ccccc4c(-c4ccc5oc6cccc7ccc4c5c76)c4ccccc34)cccc21 ; CC1(C)c2ccccc2-c2c(-c3cccc(-c4c5ccccc5c(-c5ccc6oc7cccc8ccc5c6c87)c5ccccc45)c3)cccc21 ; c1ccc2c(c1)cc(-c1ccc(-c3c4ccccc4c(-c4ccc5oc6cccc7ccc4c5c76)c4ccccc34)cc1)c1ccccc12]

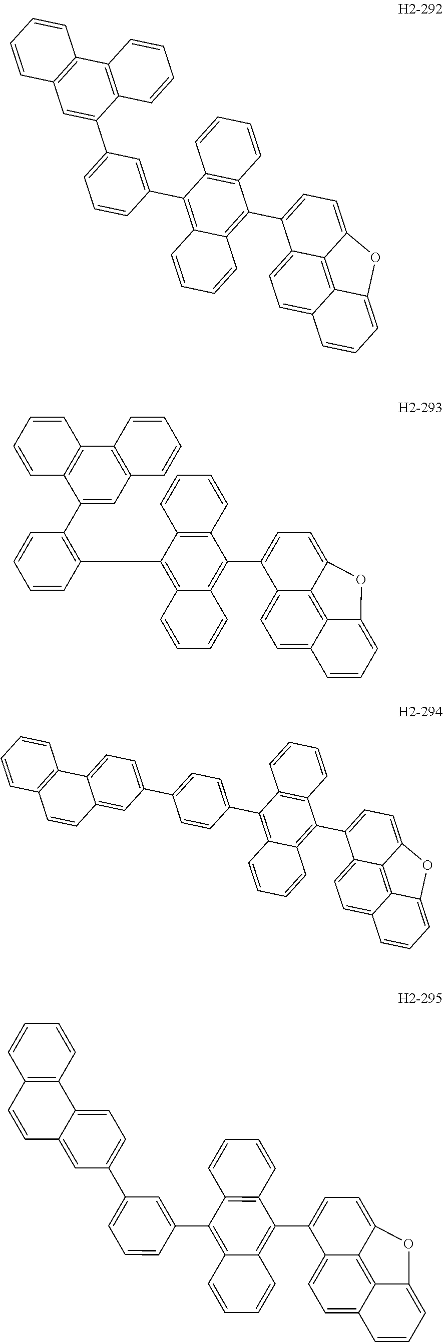 [SMILES: c1cc(-c2c3ccccc3c(-c3ccc4oc5cccc6ccc3c4c65)c3ccccc23)cc(-c2cc3ccccc3c3ccccc23)c1 ; c1ccc(-c2cc3ccccc3c3ccccc23)c(-c2c3ccccc3c(-c3ccc4oc5cccc6ccc3c4c65)c3ccccc23)c1 ; c1ccc2c(c1)ccc1cc(-c3ccc(-c4c5ccccc5c(-c5ccc6oc7cccc8ccc5c6c87)c5ccccc45)cc3)ccc12 ; c1cc(-c2ccc3c(ccc4ccccc43)c2)cc(-c2c3ccccc3c(-c3ccc4oc5cccc6ccc3c4c65)c3ccccc23)c1]

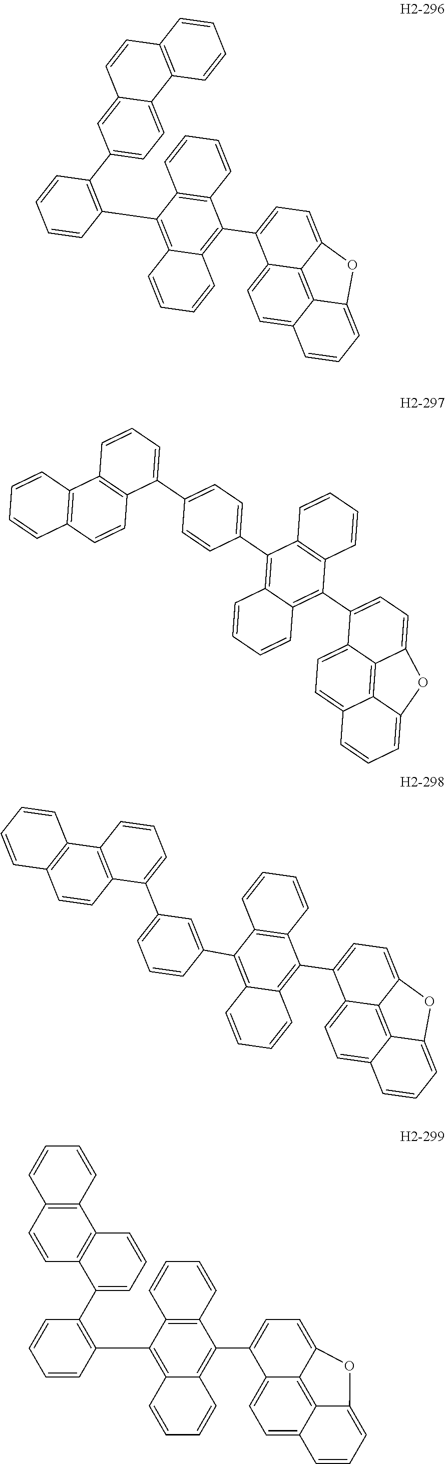 [SMILES: c1ccc(-c2c3ccccc3c(-c3ccc4oc5cccc6ccc3c4c65)c3ccccc23)c(-c2ccc3c(ccc4ccccc43)c2)c1 ; c1ccc2c(c1)ccc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2ccc2ccccc23)c1 ; c1ccc(-c2cccc3c2ccc2ccccc23)c(-c2c3ccccc3c(-c3ccc4oc5cccc6ccc3c4c65)c3ccccc23)c1]

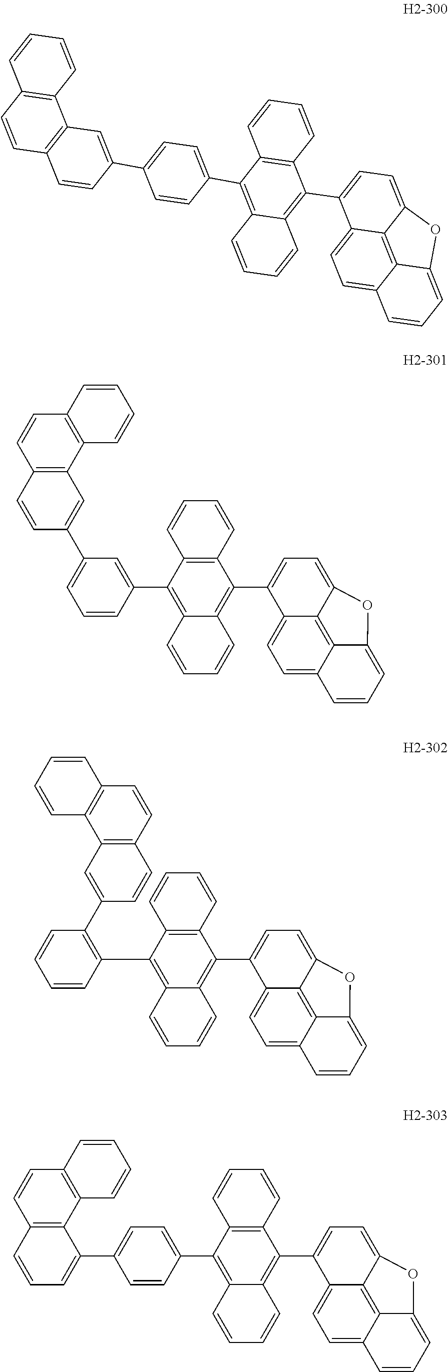 [SMILES: c1ccc2c(c1)ccc1ccc(-c3ccc(-c4c5ccccc5c(-c5ccc6oc7cccc8ccc5c6c87)c5ccccc45)cc3)cc12 ; c1cc(-c2ccc3ccc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3ccc4ccccc4c3c2)c1 ; c1ccc2c(c1)ccc1cccc(-c3ccc(-c4c5ccccc5c(-c5ccc6oc7cccc8ccc5c6c87)c5ccccc45)cc3)c12]

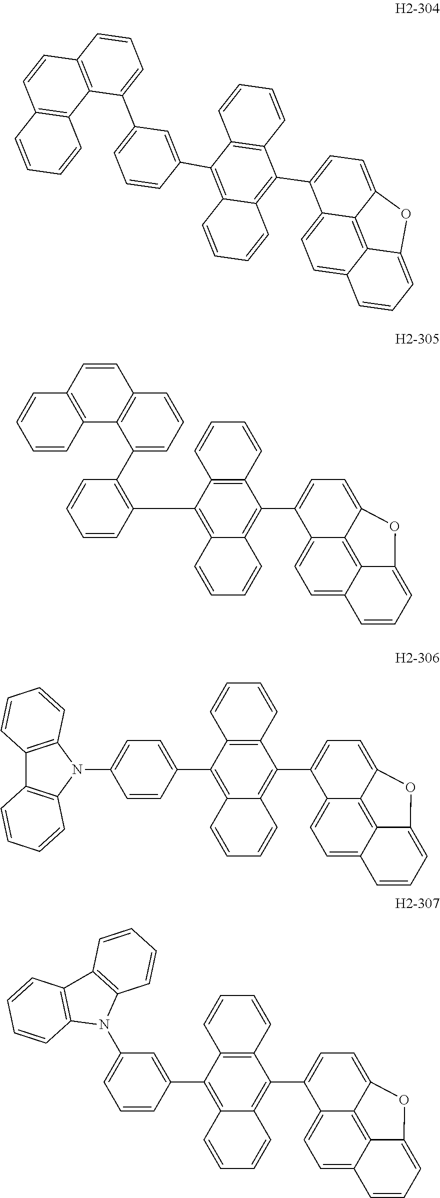 [SMILES: c1cc(-c2c3ccccc3c(-c3ccc4oc5cccc6ccc3c4c65)c3ccccc23)cc(-c2cccc3ccc4ccccc4c23)c1 ; c1ccc(-c2cccc3ccc4ccccc4c23)c(-c2c3ccccc3c(-c3ccc4oc5cccc6ccc3c4c65)c3ccccc23)c1 ; c1cc2ccc3c(-c4c5ccccc5c(-c5ccc(-n6c7ccccc7c7ccccc76)cc5)c5ccccc45)ccc4oc(c1)c2c43 ; c1cc(-c2c3ccccc3c(-c3ccc4oc5cccc6ccc3c4c65)c3ccccc23)cc(-n2c3ccccc3c3ccccc32)c1]

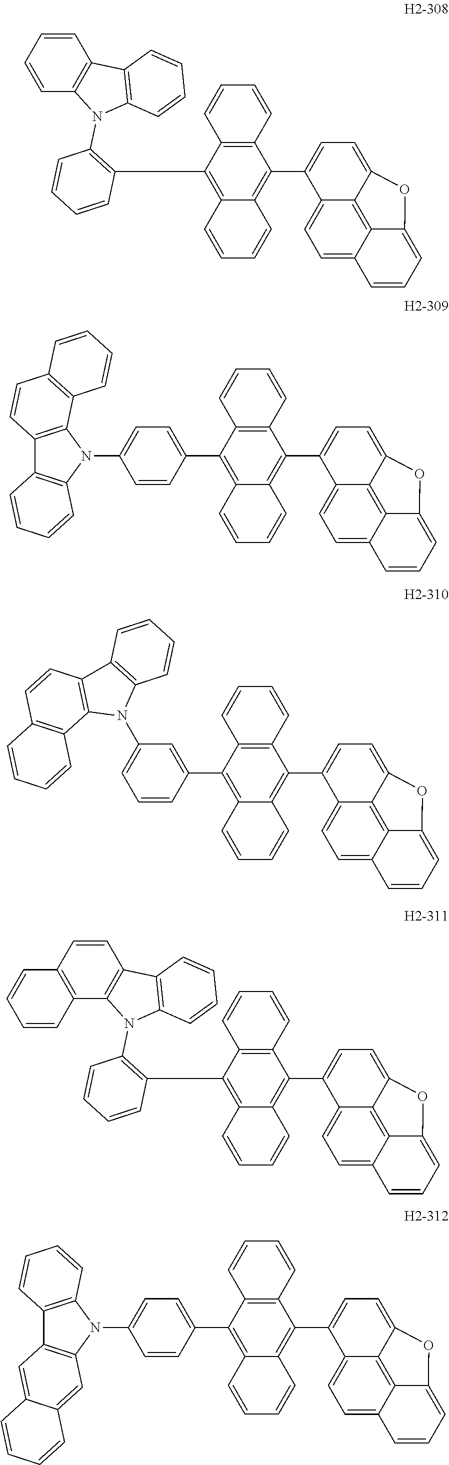 [SMILES: c1ccc(-n2c3ccccc3c3ccccc32)c(-c2c3ccccc3c(-c3ccc4oc5cccc6ccc3c4c65)c3ccccc23)c1 ; c1ccc2c(c1)ccc1c3ccccc3n(-c3ccc(-c4c5ccccc5c(-c5ccc6oc7cccc8ccc5c6c87)c5ccccc45)cc3)c21 ; c1cc(-c2c3ccccc3c(-c3ccc4oc5cccc6ccc3c4c65)c3ccccc23)cc(-n2c3ccccc3c3ccc4ccccc4c32)c1 ; c1ccc(-n2c3ccccc3c3ccc4ccccc4c32)c(-c2c3ccccc3c(-c3ccc4oc5cccc6ccc3c4c65)c3ccccc23)c1 ; c1ccc2cc3c(cc2c1)c1ccccc1n3-c1ccc(-c2c3ccccc3c(-c3ccc4oc5cccc6ccc3c4c65)c3ccccc23)cc1]

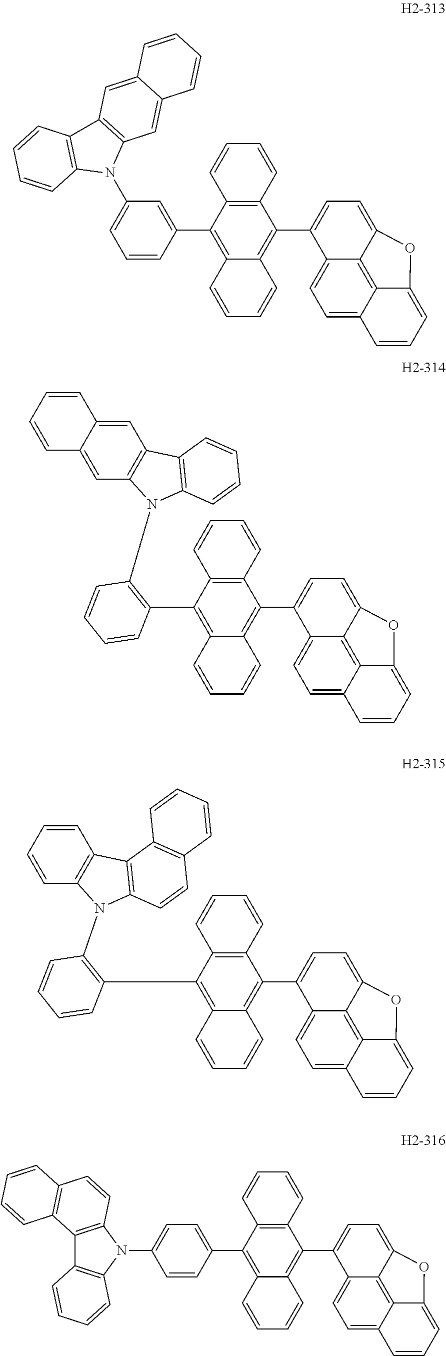 [SMILES: c1cc(-c2c3ccccc3c(-c3ccc4oc5cccc6ccc3c4c65)c3ccccc23)cc(-n2c3ccccc3c3cc4ccccc4cc32)c1 ; c1ccc(-n2c3ccccc3c3cc4ccccc4cc32)c(-c2c3ccccc3c(-c3ccc4oc5cccc6ccc3c4c65)c3ccccc23)c1 ; c1ccc(-n2c3ccccc3c3c4ccccc4ccc32)c(-c2c3ccccc3c(-c3ccc4oc5cccc6ccc3c4c65)c3ccccc23)c1 ; c1ccc2c(c1)ccc1c2c2ccccc2n1-c1ccc(-c2c3ccccc3c(-c3ccc4oc5cccc6ccc3c4c65)c3ccccc23)cc1]

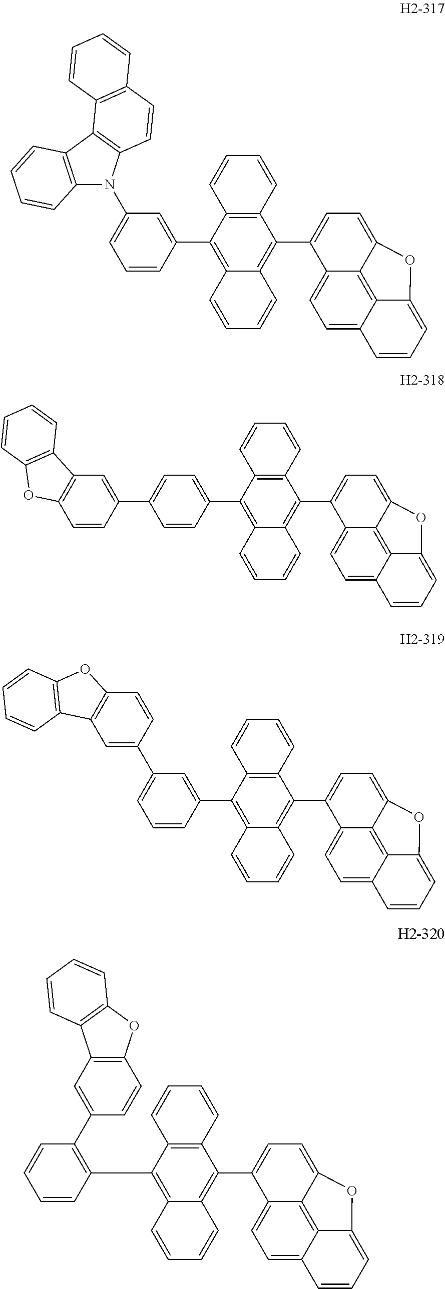 [SMILES: c1cc(-c2c3ccccc3c(-c3ccc4oc5cccc6ccc3c4c65)c3ccccc23)cc(-n2c3ccccc3c3c4ccccc4ccc32)c1 ; c1ccc2c(c1)oc1ccc(-c3ccc(-c4c5ccccc5c(-c5ccc6oc7cccc8ccc5c6c87)c5ccccc45)cc3)cc12 ; c1cc(-c2ccc3oc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3oc4ccccc4c3c2)c1]

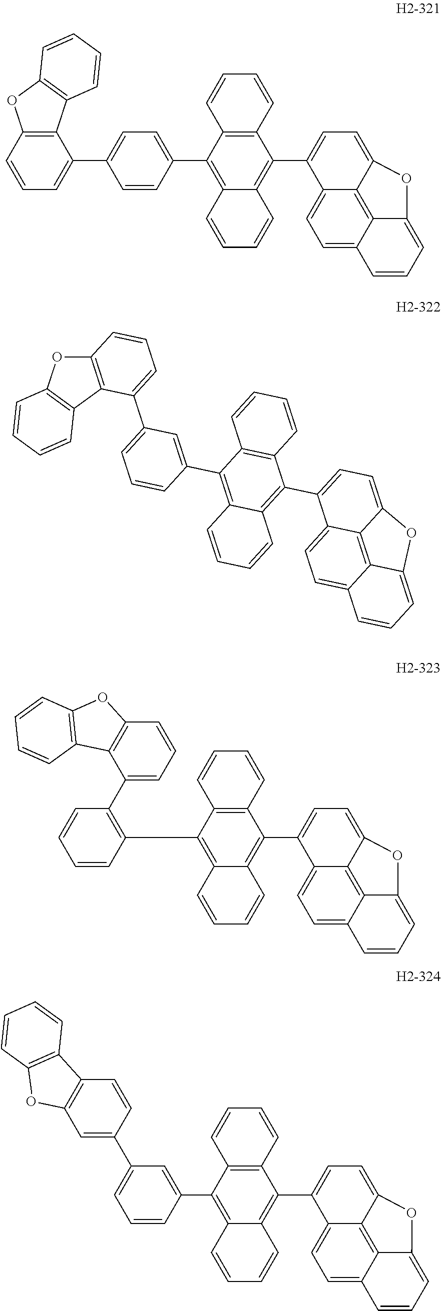 [SMILES: c1ccc2c(c1)oc1cccc(-c3ccc(-c4c5ccccc5c(-c5ccc6oc7cccc8ccc5c6c87)c5ccccc45)cc3)c12 ; c1cc(-c2c3ccccc3c(-c3ccc4oc5cccc6ccc3c4c65)c3ccccc23)cc(-c2cccc3oc4ccccc4c23)c1 ; c1ccc(-c2cccc3oc4ccccc4c23)c(-c2c3ccccc3c(-c3ccc4oc5cccc6ccc3c4c65)c3ccccc23)c1 ; c1cc(-c2ccc3c(c2)oc2ccccc23)cc(-c2c3ccccc3c(-c3ccc4oc5cccc6ccc3c4c65)c3ccccc23)c1]

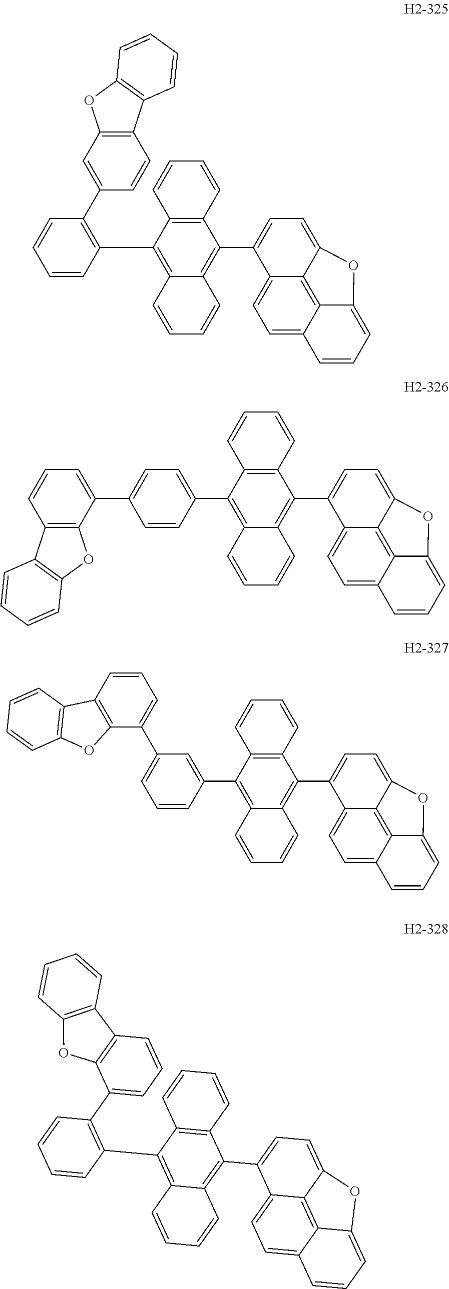 [SMILES: c1ccc(-c2c3ccccc3c(-c3ccc4oc5cccc6ccc3c4c65)c3ccccc23)c(-c2ccc3c(c2)oc2ccccc23)c1 ; c1ccc2c(c1)oc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2oc2ccccc23)c1 ; c1ccc(-c2cccc3c2oc2ccccc23)c(-c2c3ccccc3c(-c3ccc4oc5cccc6ccc3c4c65)c3ccccc23)c1]

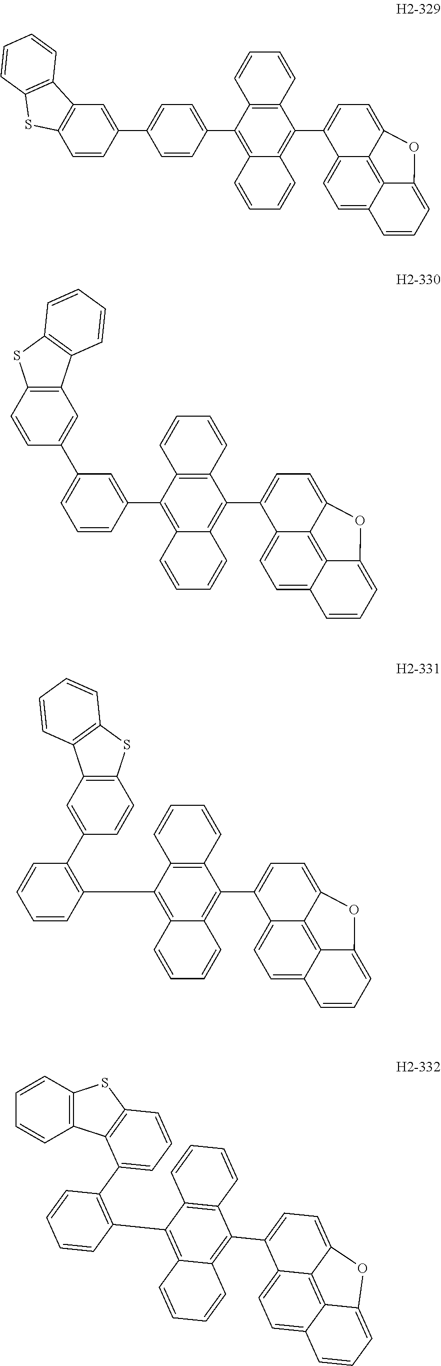 [SMILES: c1cc2ccc3c(-c4c5ccccc5c(-c5ccc(-c6ccc7sc8ccccc8c7c6)cc5)c5ccccc45)ccc4oc(c1)c2c43 ; c1cc(-c2ccc3sc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3sc4ccccc4c3c2)c1 ; c1ccc(-c2cccc3sc4ccccc4c23)c(-c2c3ccccc3c(-c3ccc4oc5cccc6ccc3c4c65)c3ccccc23)c1]

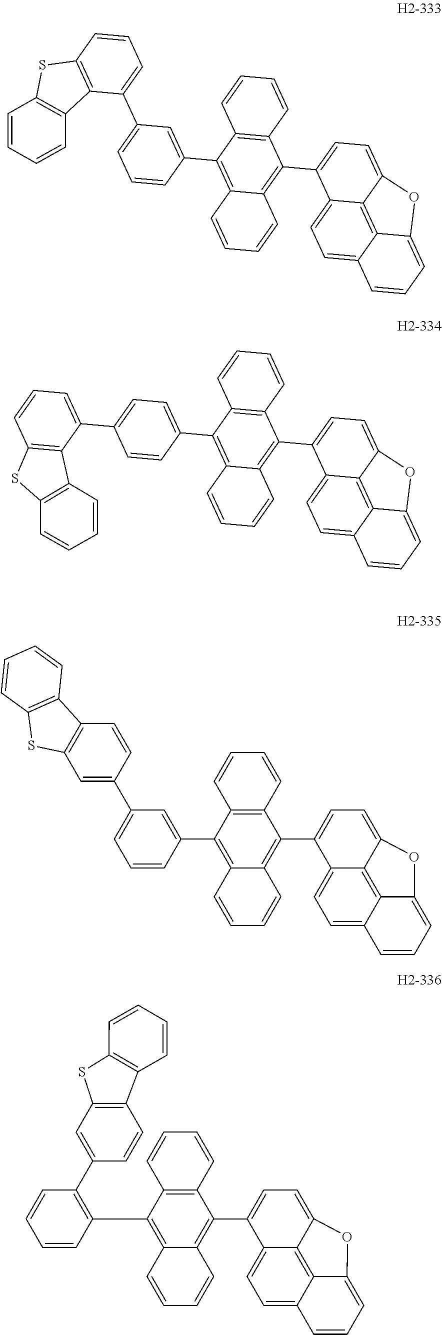 [SMILES: c1cc(-c2c3ccccc3c(-c3ccc4oc5cccc6ccc3c4c65)c3ccccc23)cc(-c2cccc3sc4ccccc4c23)c1 ; c1cc2ccc3c(-c4c5ccccc5c(-c5ccc(-c6cccc7sc8ccccc8c67)cc5)c5ccccc45)ccc4oc(c1)c2c43 ; c1cc(-c2ccc3c(c2)sc2ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(c2)sc2ccccc23)c1]

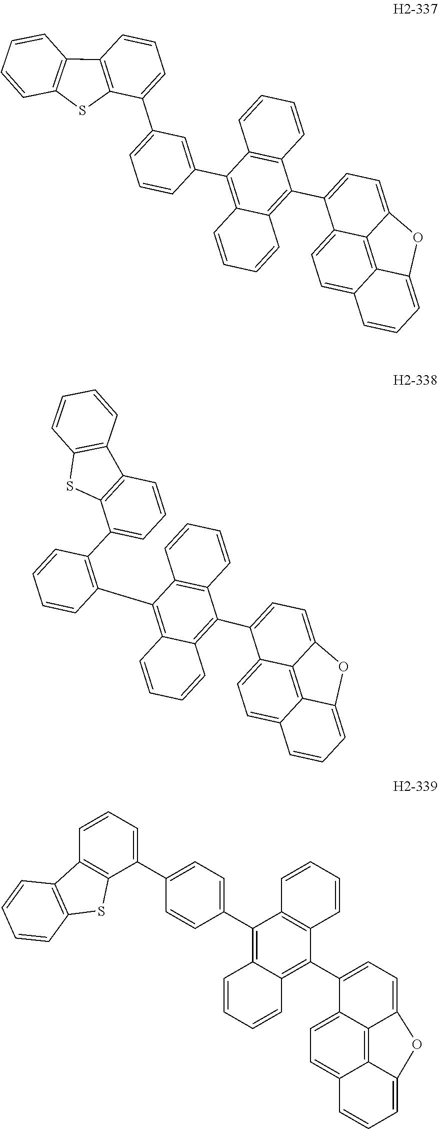 [SMILES: c1cc(-c2c3ccccc3c(-c3ccc4oc5cccc6ccc3c4c65)c3ccccc23)cc(-c2cccc3c2sc2ccccc23)c1 ; c1ccc(-c2cccc3c2sc2ccccc23)c(-c2c3ccccc3c(-c3ccc4oc5cccc6ccc3c4c65)c3ccccc23)c1 ; c1cc2ccc3c(-c4c5ccccc5c(-c5ccc(-c6cccc7c6sc6ccccc67)cc5)c5ccccc45)ccc4oc(c1)c2c43]

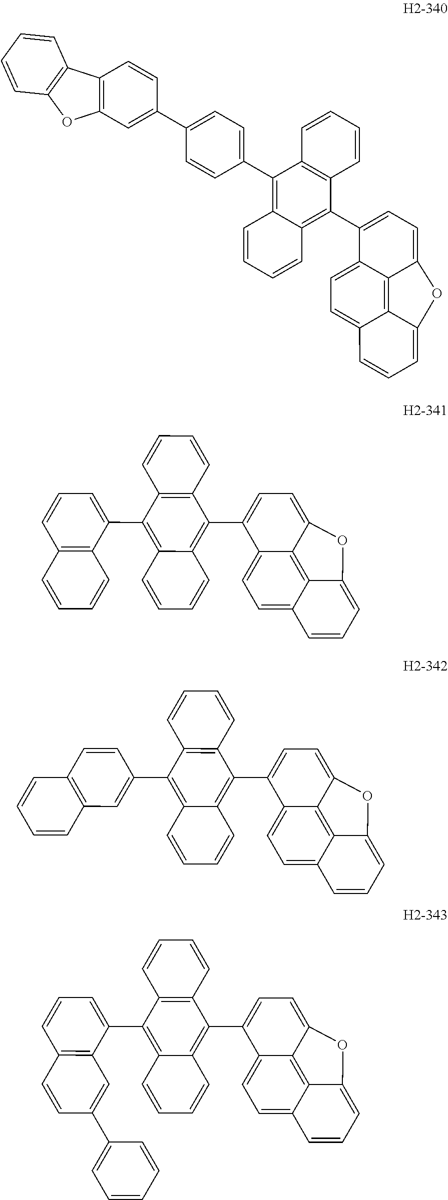 [SMILES: c1ccc2c(c1)oc1cc(-c3ccc(-c4c5ccccc5c(-c5ccc6oc7cccc8ccc5c6c87)c5ccccc45)cc3)ccc12 ; c1ccc2c(-c3c4ccccc4c(-c4ccc5oc6cccc7ccc4c5c76)c4ccccc34)cccc2c1 ; c1ccc2cc(-c3c4ccccc4c(-c4ccc5oc6cccc7ccc4c5c76)c4ccccc34)ccc2c1 ; c1ccc(-c2ccc3cccc(-c4c5ccccc5c(-c5ccc6oc7cccc8ccc5c6c87)c5ccccc45)c3c2)cc1]

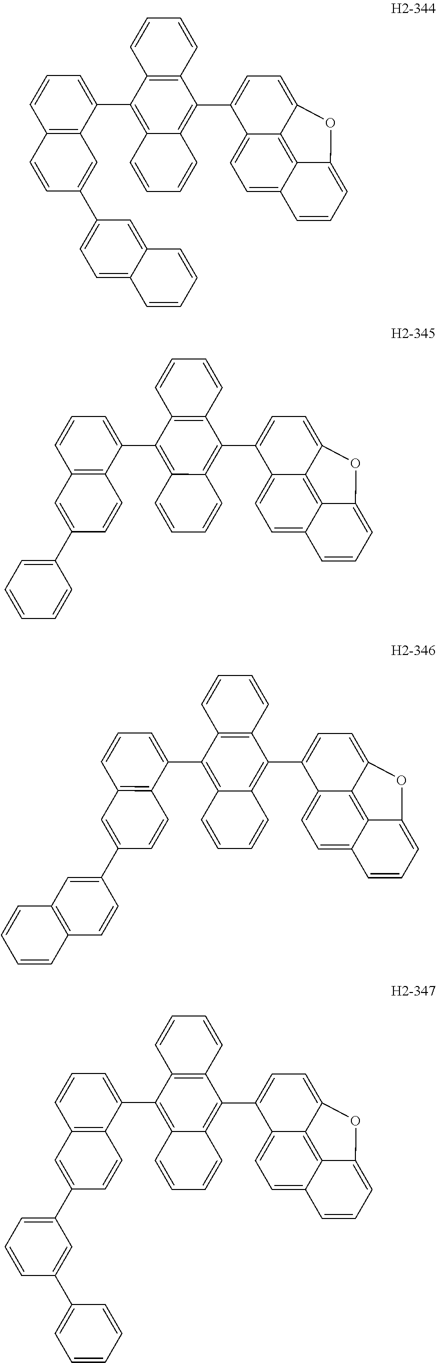 [SMILES: c1ccc2cc(-c3ccc4cccc(-c5c6ccccc6c(-c6ccc7oc8cccc9ccc6c7c98)c6ccccc56)c4c3)ccc2c1 ; c1ccc(-c2ccc3c(-c4c5ccccc5c(-c5ccc6oc7cccc8ccc5c6c87)c5ccccc45)cccc3c2)cc1 ; c1ccc2cc(-c3ccc4c(-c5c6ccccc6c(-c6ccc7oc8cccc9ccc6c7c98)c6ccccc56)cccc4c3)ccc2c1 ; c1ccc(-c2cccc(-c3ccc4c(-c5c6ccccc6c(-c6ccc7oc8cccc9ccc6c7c98)c6ccccc56)cccc4c3)c2)cc1]

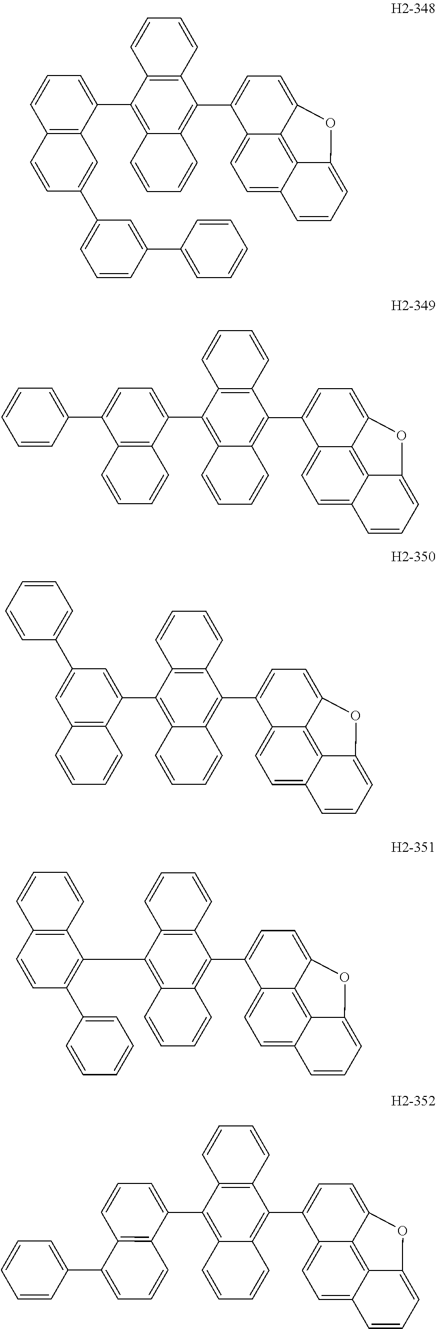 [SMILES: c1ccc(-c2cccc(-c3ccc4cccc(-c5c6ccccc6c(-c6ccc7oc8cccc9ccc6c7c98)c6ccccc56)c4c3)c2)cc1 ; c1ccc(-c2ccc(-c3c4ccccc4c(-c4ccc5oc6cccc7ccc4c5c76)c4ccccc34)c3ccccc23)cc1 ; c1ccc(-c2cc(-c3c4ccccc4c(-c4ccc5oc6cccc7ccc4c5c76)c4ccccc34)c3ccccc3c2)cc1 ; c1ccc(-c2ccc3ccccc3c2-c2c3ccccc3c(-c3ccc4oc5cccc6ccc3c4c65)c3ccccc23)cc1 ; c1ccc(-c2cccc3c(-c4c5ccccc5c(-c5ccc6oc7cccc8ccc5c6c87)c5ccccc45)cccc23)cc1]

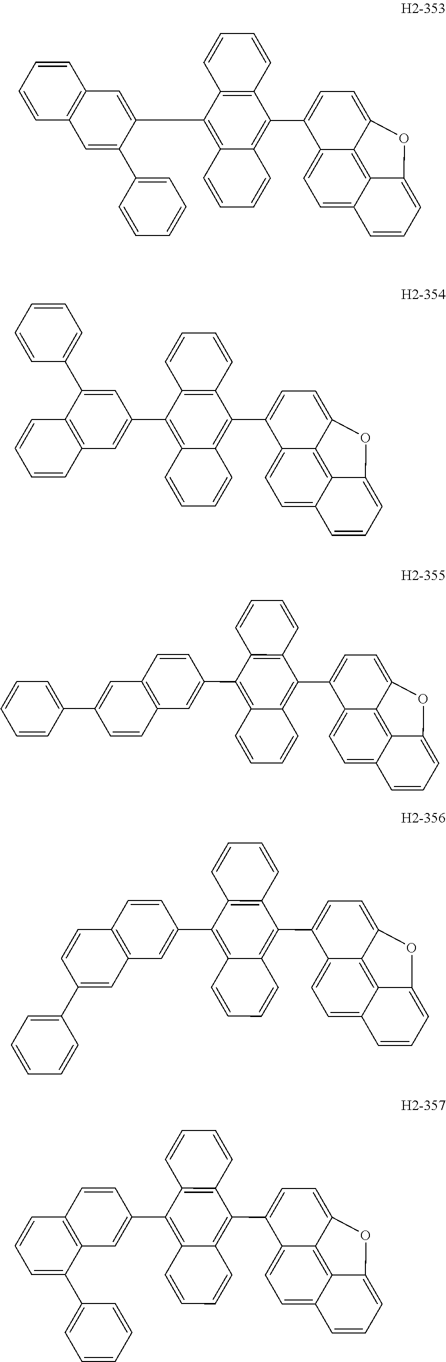 [SMILES: c1ccc(-c2cc3ccccc3cc2-c2c3ccccc3c(-c3ccc4oc5cccc6ccc3c4c65)c3ccccc23)cc1 ; c1ccc(-c2cc(-c3c4ccccc4c(-c4ccc5oc6cccc7ccc4c5c76)c4ccccc34)cc3ccccc23)cc1 ; c1ccc(-c2ccc3cc(-c4c5ccccc5c(-c5ccc6oc7cccc8ccc5c6c87)c5ccccc45)ccc3c2)cc1 ; c1ccc(-c2ccc3ccc(-c4c5ccccc5c(-c5ccc6oc7cccc8ccc5c6c87)c5ccccc45)cc3c2)cc1 ; c1ccc(-c2cccc3ccc(-c4c5ccccc5c(-c5ccc6oc7cccc8ccc5c6c87)c5ccccc45)cc23)cc1]

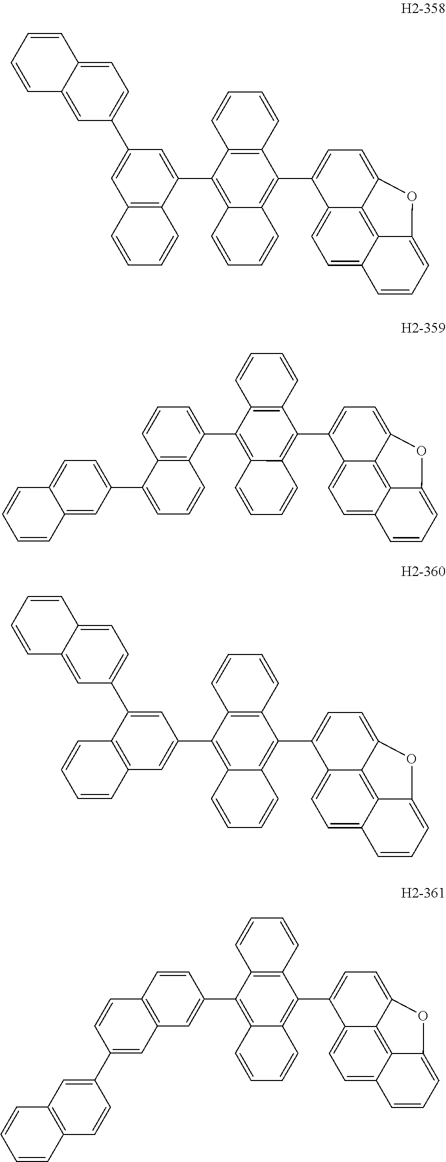 [SMILES: c1ccc2cc(-c3cc(-c4c5ccccc5c(-c5ccc6oc7cccc8ccc5c6c87)c5ccccc45)c4ccccc4c3)ccc2c1 ; c1ccc2cc(-c3cccc4c(-c5c6ccccc6c(-c6ccc7oc8cccc9ccc6c7c98)c6ccccc56)cccc34)ccc2c1 ; c1ccc2cc(-c3cc(-c4c5ccccc5c(-c5ccc6oc7cccc8ccc5c6c87)c5ccccc45)cc4ccccc34)ccc2c1 ; c1ccc2cc(-c3ccc4ccc(-c5c6ccccc6c(-c6ccc7oc8cccc9ccc6c7c98)c6ccccc56)cc4c3)ccc2c1]

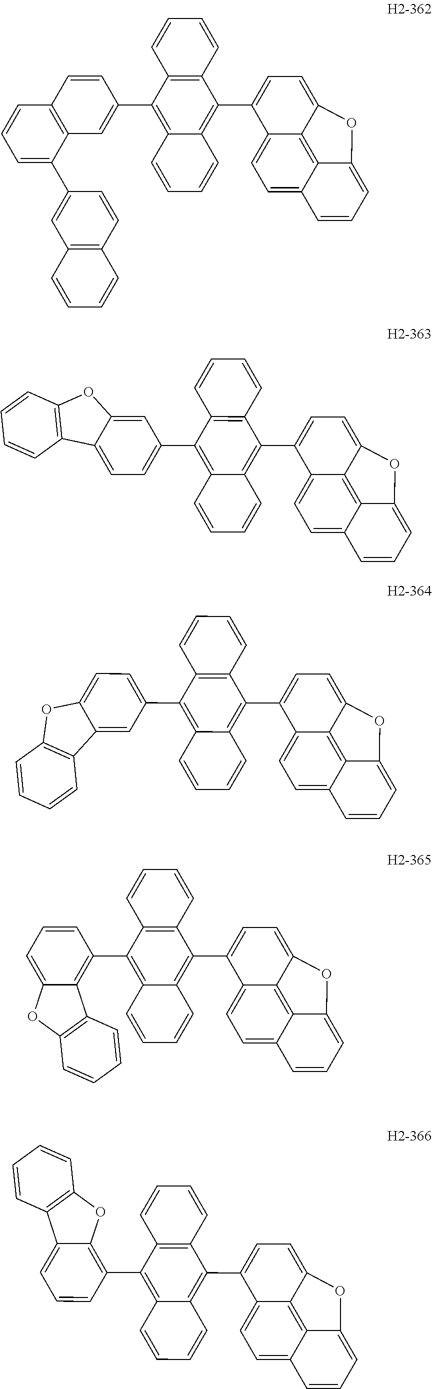 [SMILES: c1ccc2cc(-c3cccc4ccc(-c5c6ccccc6c(-c6ccc7oc8cccc9ccc6c7c98)c6ccccc56)cc34)ccc2c1 ; c1ccc2c(c1)oc1cc(-c3c4ccccc4c(-c4ccc5oc6cccc7ccc4c5c76)c4ccccc34)ccc12 ; c1ccc2c(c1)oc1ccc(-c3c4ccccc4c(-c4ccc5oc6cccc7ccc4c5c76)c4ccccc34)cc12 ; c1ccc2c(c1)oc1cccc(-c3c4ccccc4c(-c4ccc5oc6cccc7ccc4c5c76)c4ccccc34)c12 ; c1ccc2c(c1)oc1c(-c3c4ccccc4c(-c4ccc5oc6cccc7ccc4c5c76)c4ccccc34)cccc12]

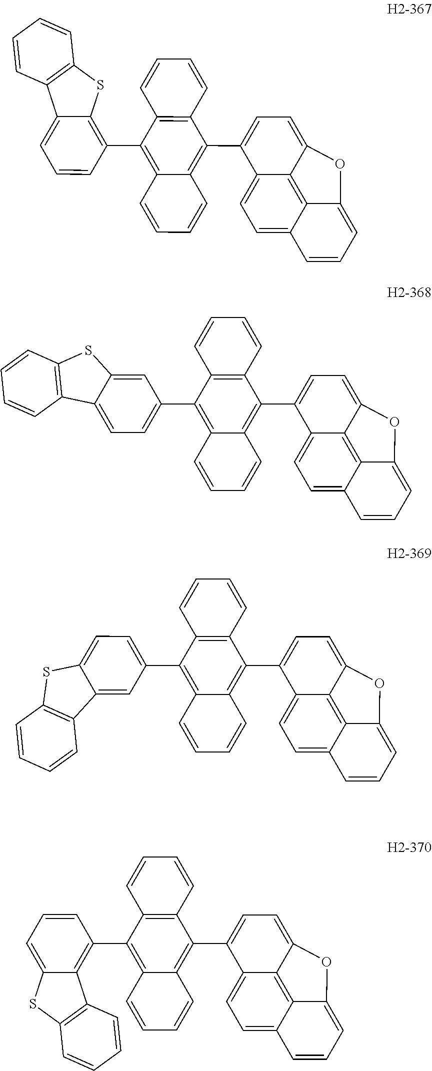 [SMILES: c1cc2ccc3c(-c4c5ccccc5c(-c5cccc6c5sc5ccccc56)c5ccccc45)ccc4oc(c1)c2c43 ; c1cc2ccc3c(-c4c5ccccc5c(-c5ccc6c(c5)sc5ccccc56)c5ccccc45)ccc4oc(c1)c2c43 ; c1cc2ccc3c(-c4c5ccccc5c(-c5ccc6sc7ccccc7c6c5)c5ccccc45)ccc4oc(c1)c2c43 ; c1cc2ccc3c(-c4c5ccccc5c(-c5cccc6sc7ccccc7c56)c5ccccc45)ccc4oc(c1)c2c43]

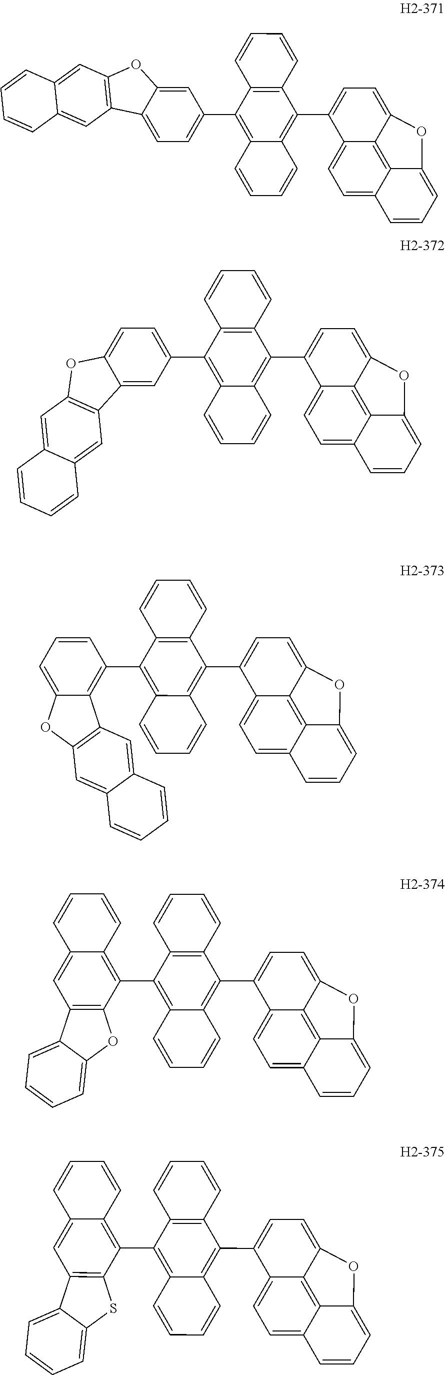 [SMILES: c1ccc2cc3c(cc2c1)oc1cc(-c2c4ccccc4c(-c4ccc5oc6cccc7ccc4c5c76)c4ccccc24)ccc13 ; c1ccc2cc3c(cc2c1)oc1ccc(-c2c4ccccc4c(-c4ccc5oc6cccc7ccc4c5c76)c4ccccc24)cc13 ; c1ccc2cc3c(cc2c1)oc1cccc(-c2c4ccccc4c(-c4ccc5oc6cccc7ccc4c5c76)c4ccccc24)c13 ; c1ccc2c(-c3c4ccccc4c(-c4ccc5oc6cccc7ccc4c5c76)c4ccccc34)c3oc4ccccc4c3cc2c1 ; c1ccc2c(-c3c4ccccc4c(-c4ccc5oc6cccc7ccc4c5c76)c4ccccc34)c3sc4ccccc4c3cc2c1]

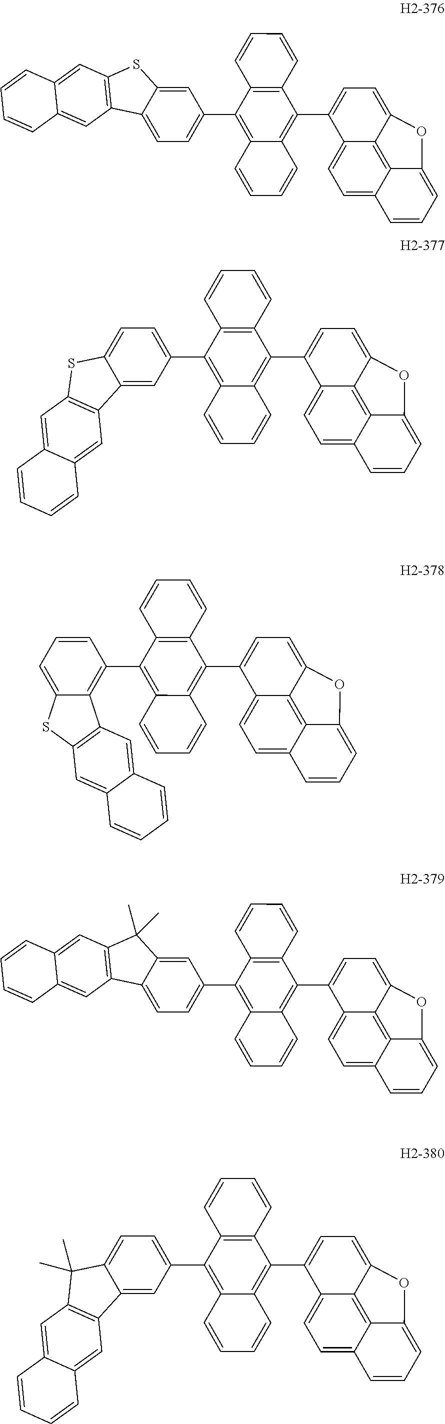 [SMILES: c1ccc2cc3c(cc2c1)sc1cc(-c2c4ccccc4c(-c4ccc5oc6cccc7ccc4c5c76)c4ccccc24)ccc13 ; c1ccc2cc3c(cc2c1)sc1ccc(-c2c4ccccc4c(-c4ccc5oc6cccc7ccc4c5c76)c4ccccc24)cc13 ; c1ccc2cc3c(cc2c1)sc1cccc(-c2c4ccccc4c(-c4ccc5oc6cccc7ccc4c5c76)c4ccccc24)c13 ; CC1(C)c2cc(-c3c4ccccc4c(-c4ccc5oc6cccc7ccc4c5c76)c4ccccc34)ccc2-c2cc3ccccc3cc21 ; CC1(C)c2ccc(-c3c4ccccc4c(-c4ccc5oc6cccc7ccc4c5c76)c4ccccc34)cc2-c2cc3ccccc3cc21]

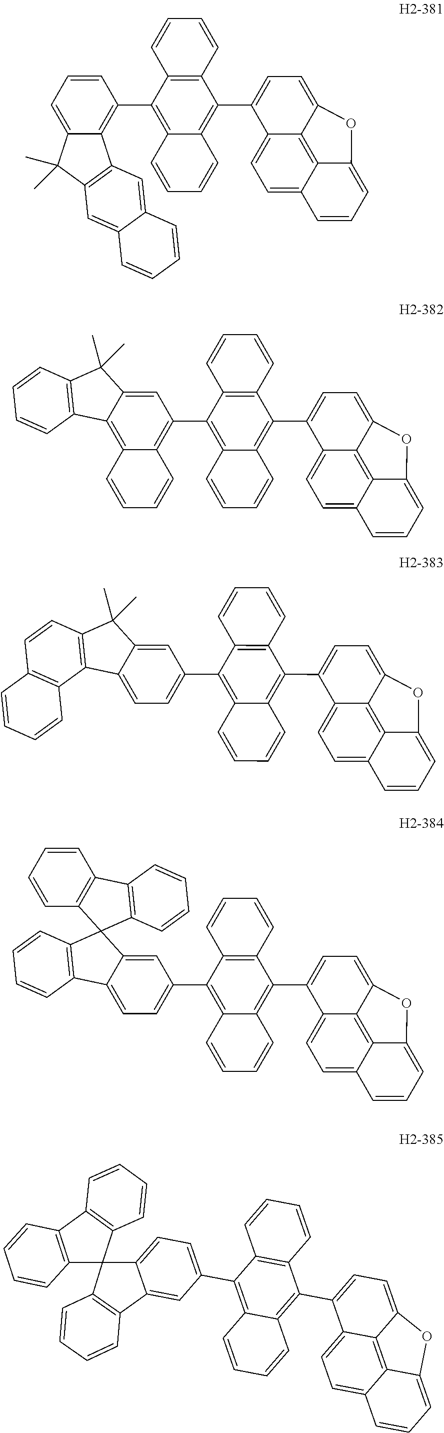 [SMILES: CC1(C)c2cc3ccccc3cc2-c2c(-c3c4ccccc4c(-c4ccc5oc6cccc7ccc4c5c76)c4ccccc34)cccc21 ; CC1(C)c2ccccc2-c2c1cc(-c1c3ccccc3c(-c3ccc4oc5cccc6ccc3c4c65)c3ccccc13)c1ccccc21 ; CC1(C)c2cc(-c3c4ccccc4c(-c4ccc5oc6cccc7ccc4c5c76)c4ccccc34)ccc2-c2c1ccc1ccccc21 ; c1ccc2c(c1)-c1ccccc1C21c2ccccc2-c2ccc(-c3c4ccccc4c(-c4ccc5oc6cccc7ccc4c5c76)c4ccccc34)cc21 ; c1ccc2c(c1)-c1ccccc1C21c2ccccc2-c2cc(-c3c4ccccc4c(-c4ccc5oc6cccc7ccc4c5c76)c4ccccc34)ccc21]

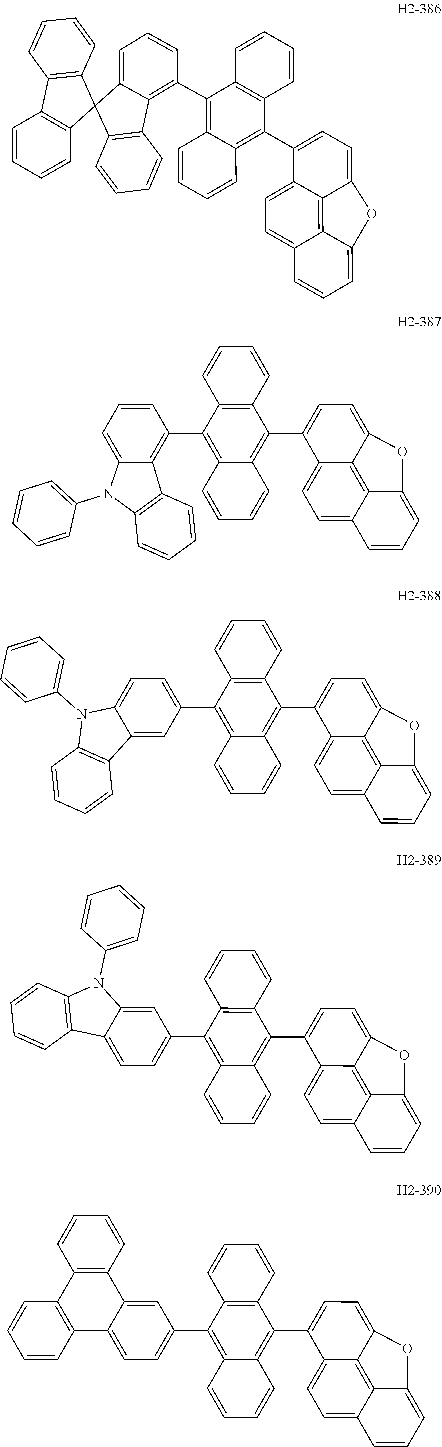 [SMILES: c1ccc2c(c1)-c1ccccc1C21c2ccccc2-c2c(-c3c4ccccc4c(-c4ccc5oc6cccc7ccc4c5c76)c4ccccc34)cccc21 ; c1ccc(-n2c3ccccc3c3c(-c4c5ccccc5c(-c5ccc6oc7cccc8ccc5c6c87)c5ccccc45)cccc32)cc1 ; c1ccc(-n2c3ccccc3c3cc(-c4c5ccccc5c(-c5ccc6oc7cccc8ccc5c6c87)c5ccccc45)ccc32)cc1 ; c1ccc(-n2c3ccccc3c3ccc(-c4c5ccccc5c(-c5ccc6oc7cccc8ccc5c6c87)c5ccccc45)cc32)cc1 ; c1cc2ccc3c(-c4c5ccccc5c(-c5ccc6c7ccccc7c7ccccc7c6c5)c5ccccc45)ccc4oc(c1)c2c43]

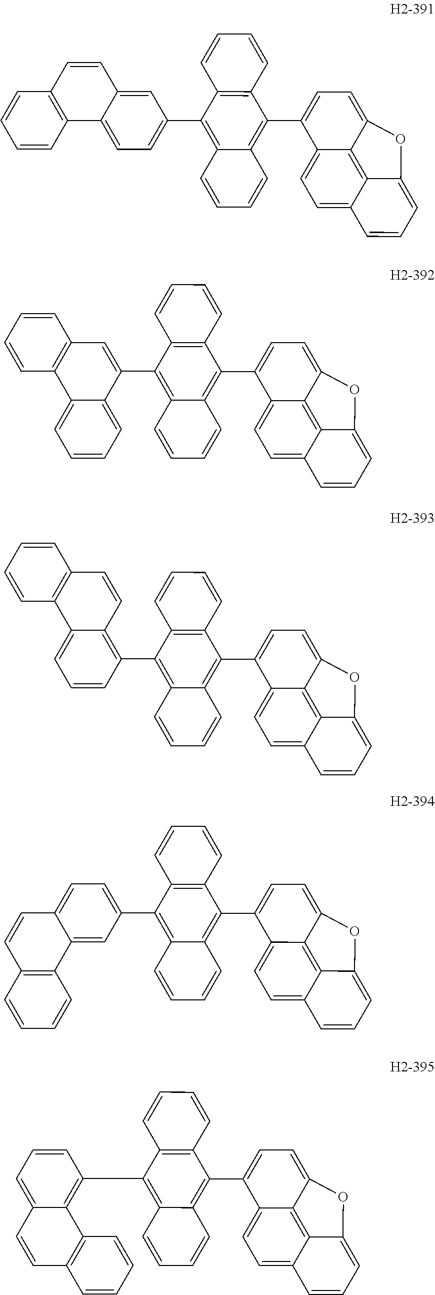 [SMILES: c1ccc2c(c1)ccc1cc(-c3c4ccccc4c(-c4ccc5oc6cccc7ccc4c5c76)c4ccccc34)ccc12 ; c1ccc2c(c1)cc(-c1c3ccccc3c(-c3ccc4oc5cccc6ccc3c4c65)c3ccccc13)c1ccccc12 ; c1ccc2c(c1)ccc1c(-c3c4ccccc4c(-c4ccc5oc6cccc7ccc4c5c76)c4ccccc34)cccc12 ; c1ccc2c(c1)ccc1ccc(-c3c4ccccc4c(-c4ccc5oc6cccc7ccc4c5c76)c4ccccc34)cc12 ; c1ccc2c(c1)ccc1cccc(-c3c4ccccc4c(-c4ccc5oc6cccc7ccc4c5c76)c4ccccc34)c12]

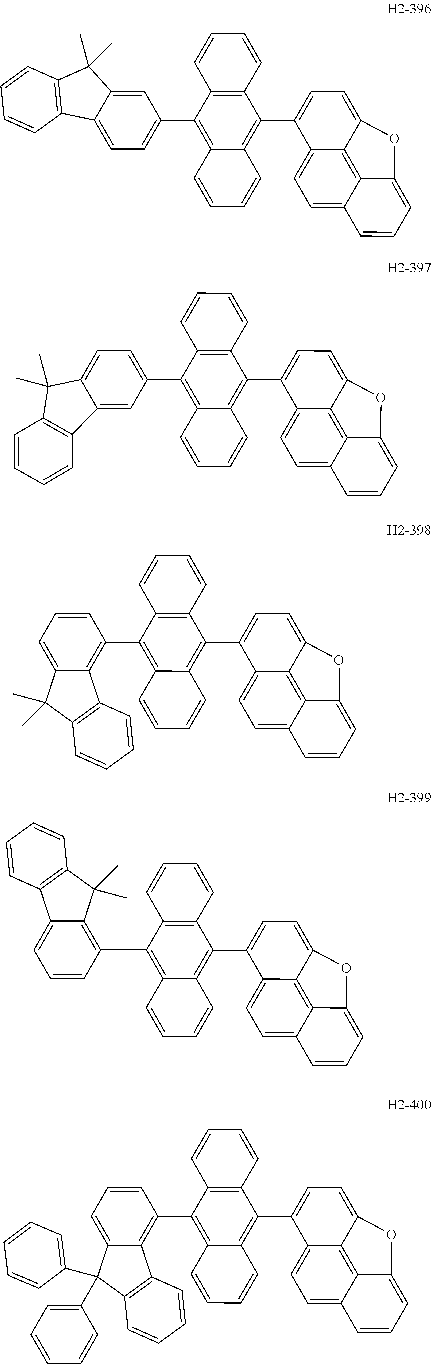 [SMILES: CC1(C)c2ccccc2-c2ccc(-c3c4ccccc4c(-c4ccc5oc6cccc7ccc4c5c76)c4ccccc34)cc21 ; CC1(C)c2ccccc2-c2cc(-c3c4ccccc4c(-c4ccc5oc6cccc7ccc4c5c76)c4ccccc34)ccc21 ; CC1(C)c2ccccc2-c2c(-c3c4ccccc4c(-c4ccc5oc6cccc7ccc4c5c76)c4ccccc34)cccc21 ; CC1(C)c2ccccc2-c2cccc(-c3c4ccccc4c(-c4ccc5oc6cccc7ccc4c5c76)c4ccccc34)c21 ; c1ccc(C2(c3ccccc3)c3ccccc3-c3c(-c4c5ccccc5c(-c5ccc6oc7cccc8ccc5c6c87)c5ccccc45)cccc32)cc1]

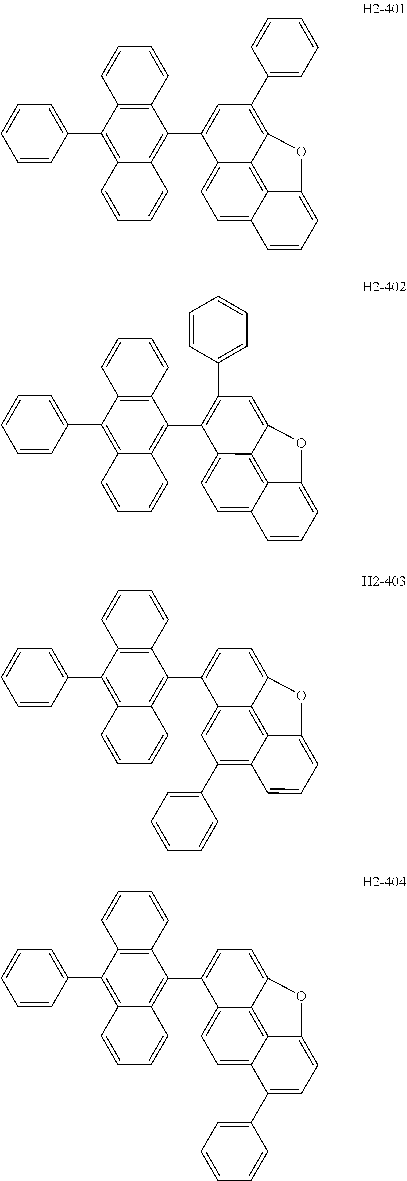

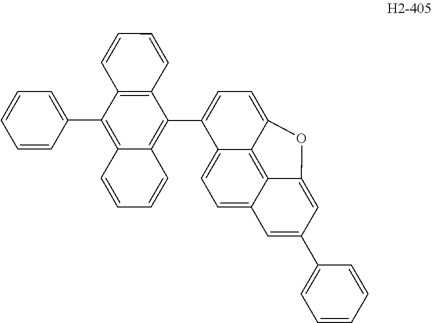 [SMILES: c1ccc(-c2cc3ccc4c(-c5c6ccccc6c(-c6ccccc6)c6ccccc56)ccc5oc(c2)c3c54)cc1]

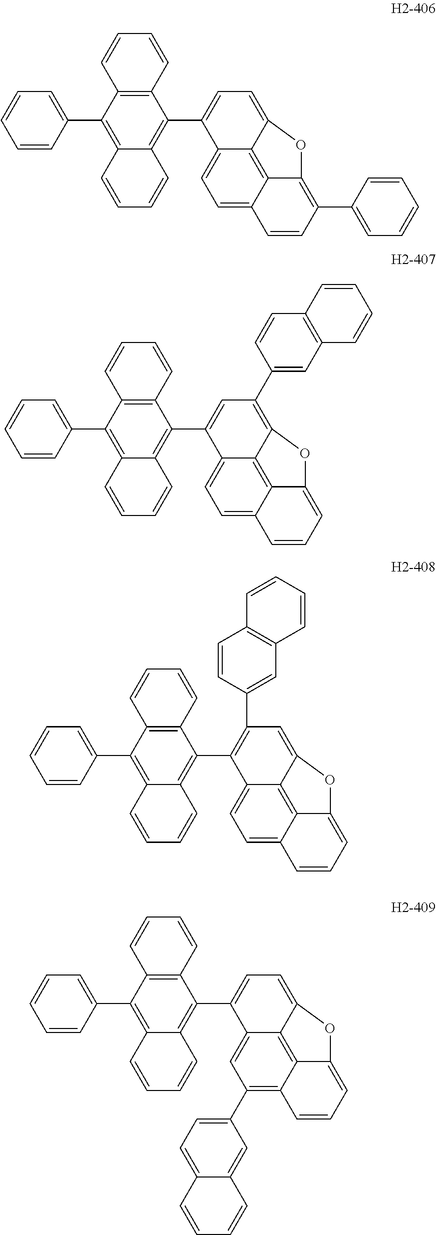

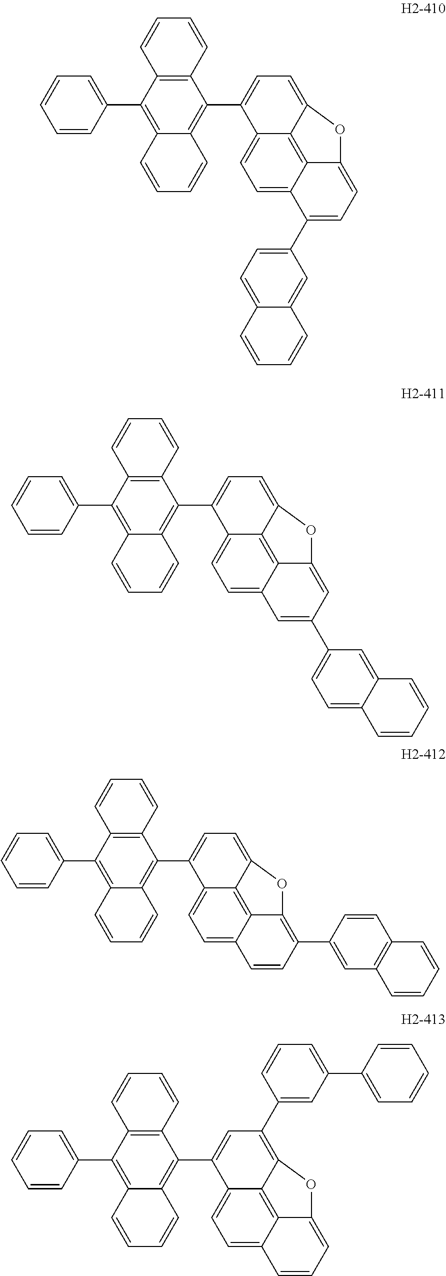 [SMILES: c1ccc(-c2c3ccccc3c(-c3ccc4oc5ccc(-c6ccc7ccccc7c6)c6ccc3c4c56)c3ccccc23)cc1 ; c1ccc(-c2c3ccccc3c(-c3ccc4oc5cc(-c6ccc7ccccc7c6)cc6ccc3c4c65)c3ccccc23)cc1 ; c1ccc(-c2c3ccccc3c(-c3ccc4oc5c(-c6ccc7ccccc7c6)ccc6ccc3c4c65)c3ccccc23)cc1 ; c1ccc(-c2cccc(-c3cc(-c4c5ccccc5c(-c5ccccc5)c5ccccc45)c4ccc5cccc6oc3c4c56)c2)cc1]

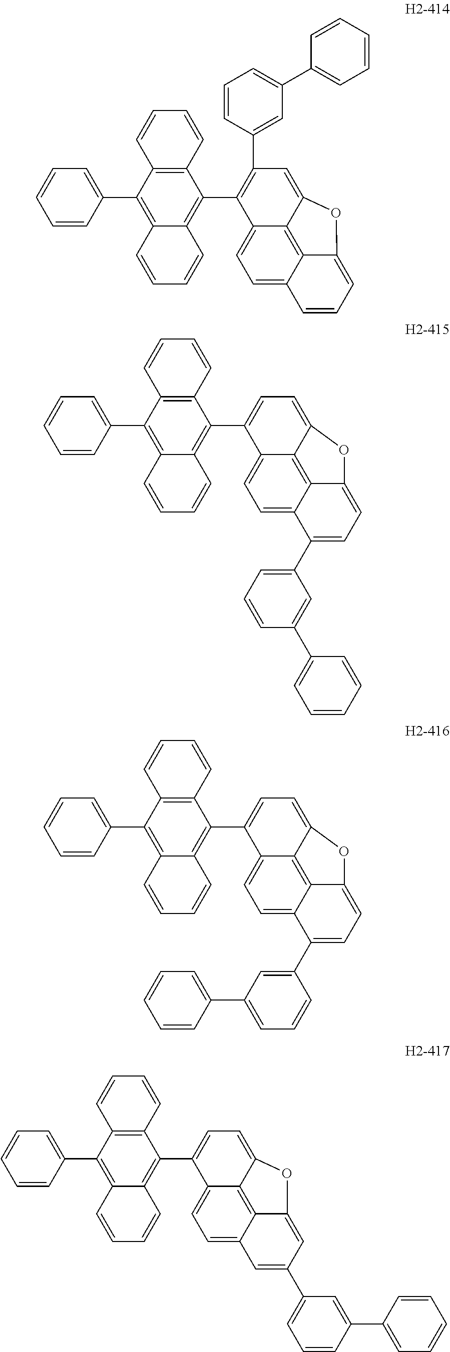 [SMILES: c1ccc(-c2cccc(-c3cc4oc5cccc6ccc(c3-c3c7ccccc7c(-c7ccccc7)c7ccccc37)c4c65)c2)cc1 ; c1ccc(-c2cccc(-c3ccc4oc5ccc(-c6c7ccccc7c(-c7ccccc7)c7ccccc67)c6ccc3c4c56)c2)cc1 ; c1ccc(-c2cccc(-c3ccc4oc5ccc(-c6c7ccccc7c(-c7ccccc7)c7ccccc67)c6ccc3c4c56)c2)cc1 ; c1ccc(-c2cccc(-c3cc4ccc5c(-c6c7ccccc7c(-c7ccccc7)c7ccccc67)ccc6oc(c3)c4c65)c2)cc1]

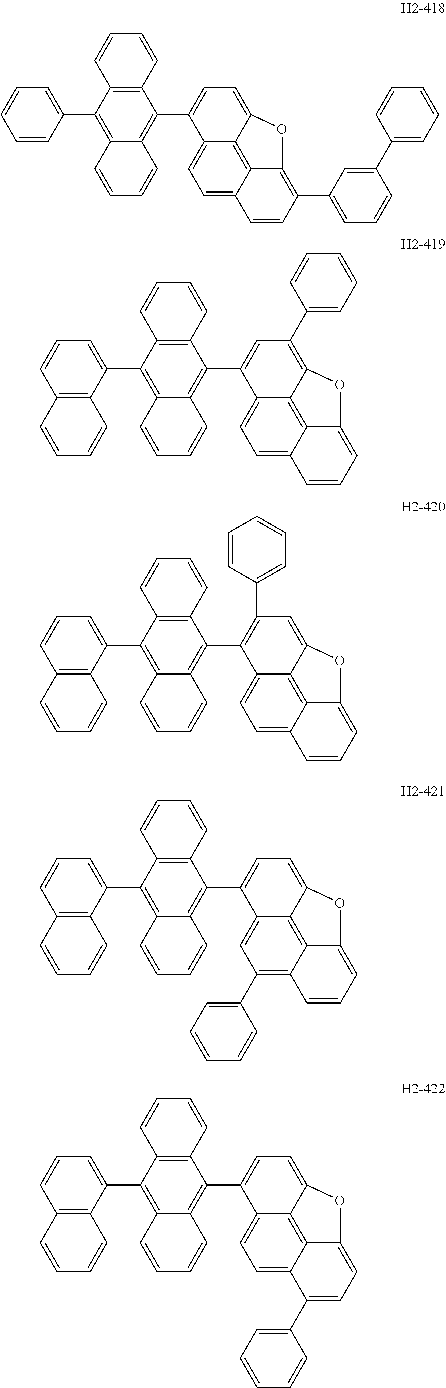

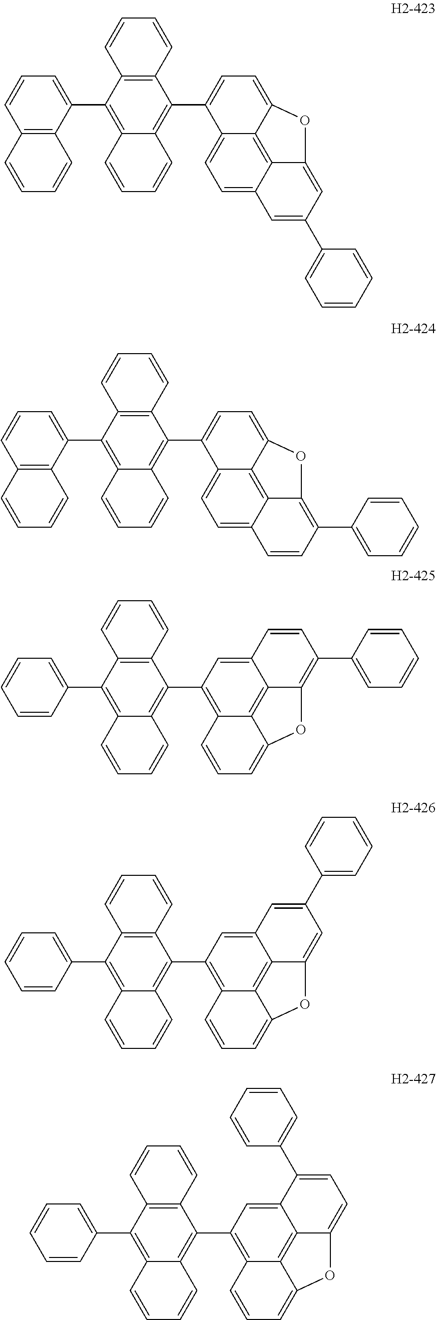

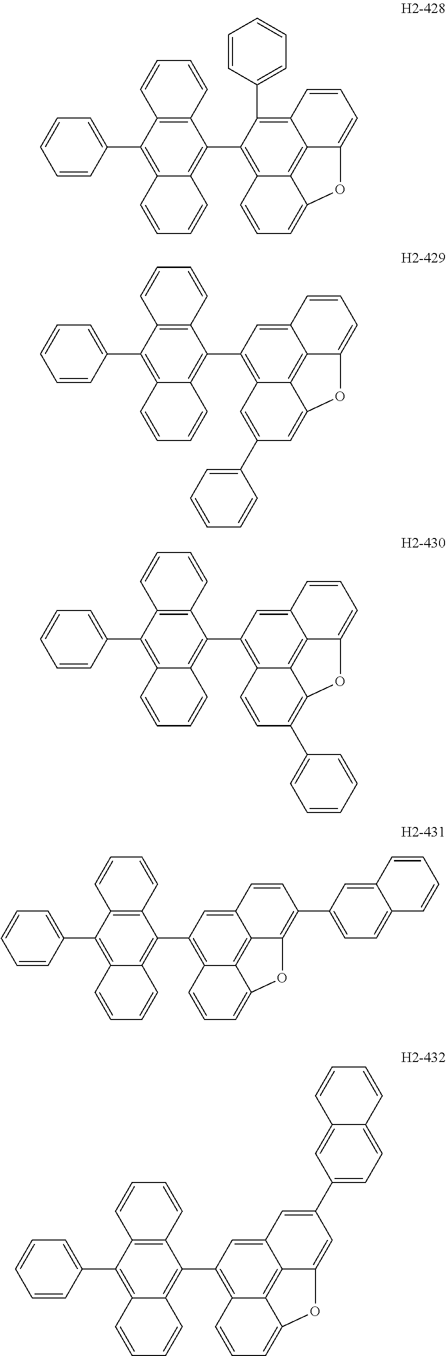

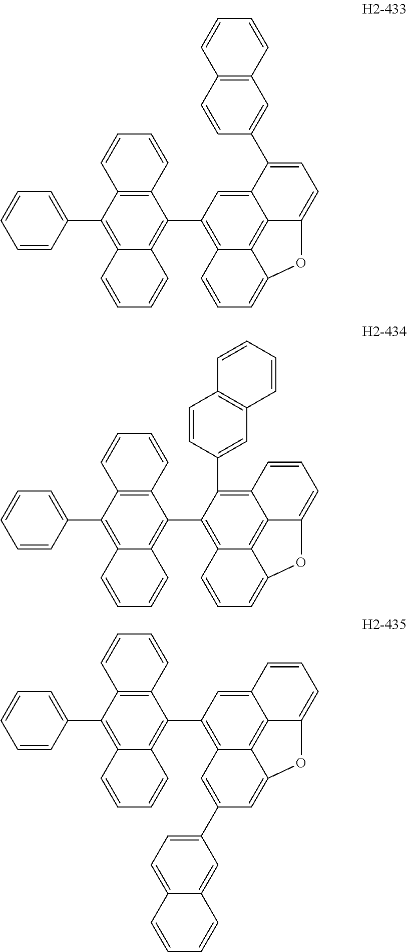

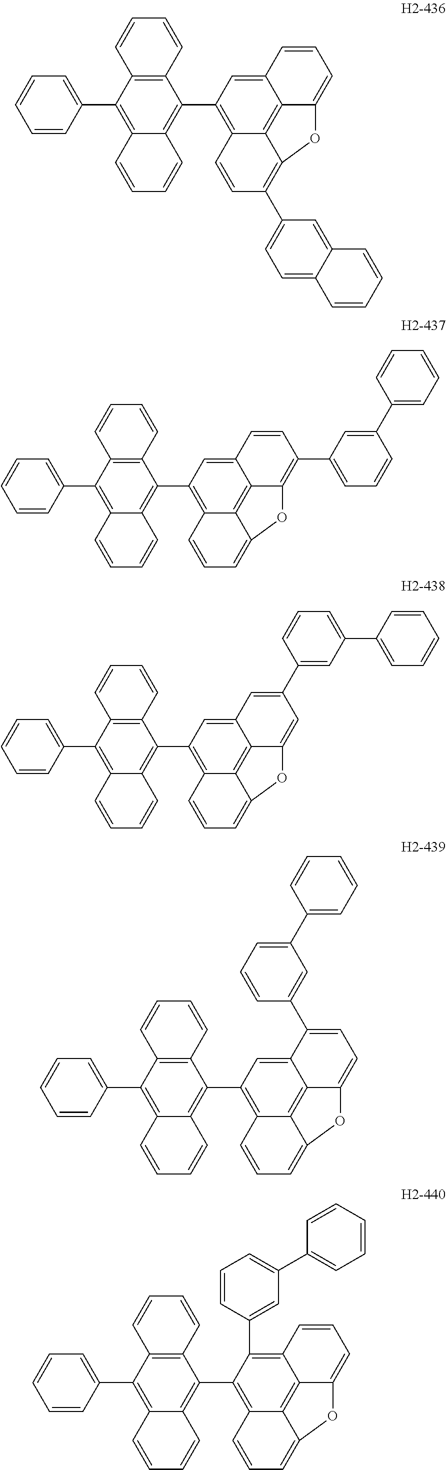 [SMILES: c1ccc(-c2c3ccccc3c(-c3cc4cccc5oc6c(-c7ccc8ccccc8c7)ccc3c6c45)c3ccccc23)cc1 ; c1ccc(-c2cccc(-c3ccc4cc(-c5c6ccccc6c(-c6ccccc6)c6ccccc56)c5cccc6oc3c4c65)c2)cc1 ; c1ccc(-c2cccc(-c3cc4cc(-c5c6ccccc6c(-c6ccccc6)c6ccccc56)c5cccc6oc(c3)c4c65)c2)cc1 ; c1ccc(-c2cccc(-c3ccc4oc5cccc6c(-c7c8ccccc8c(-c8ccccc8)c8ccccc78)cc3c4c56)c2)cc1 ; c1ccc(-c2cccc(-c3c(-c4c5ccccc5c(-c5ccccc5)c5ccccc45)c4cccc5oc6cccc3c6c54)c2)cc1]

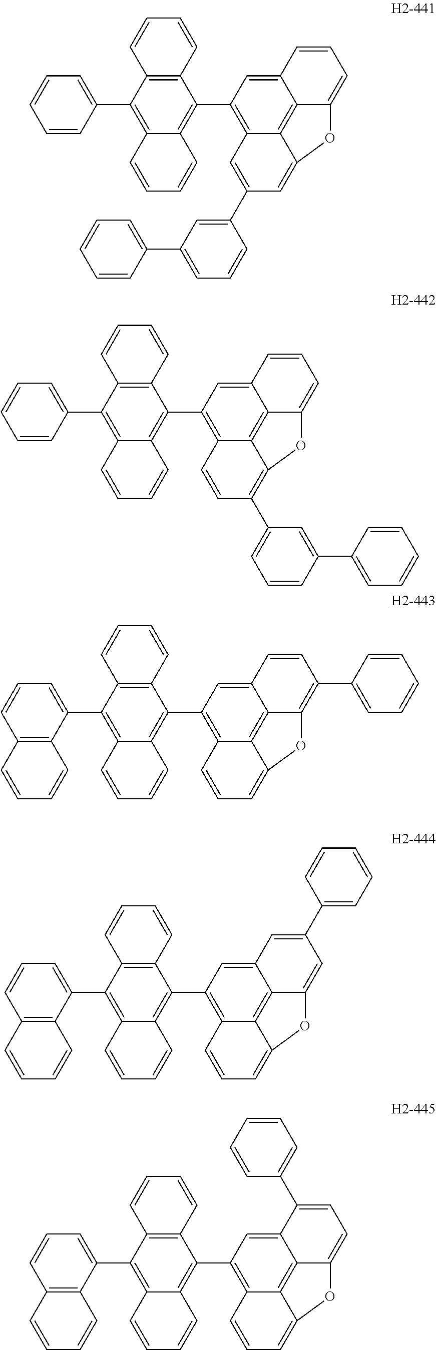

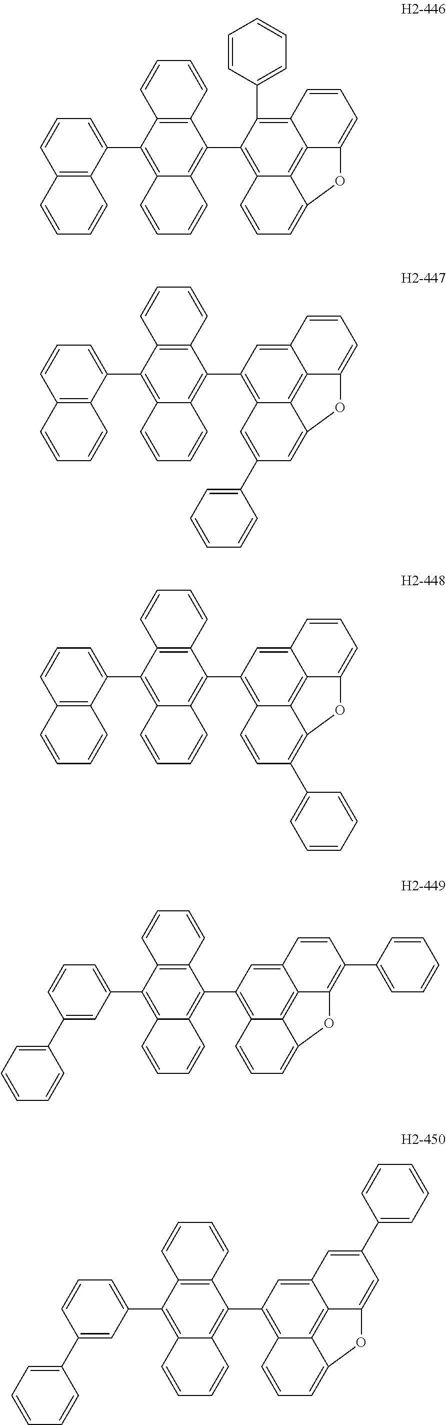

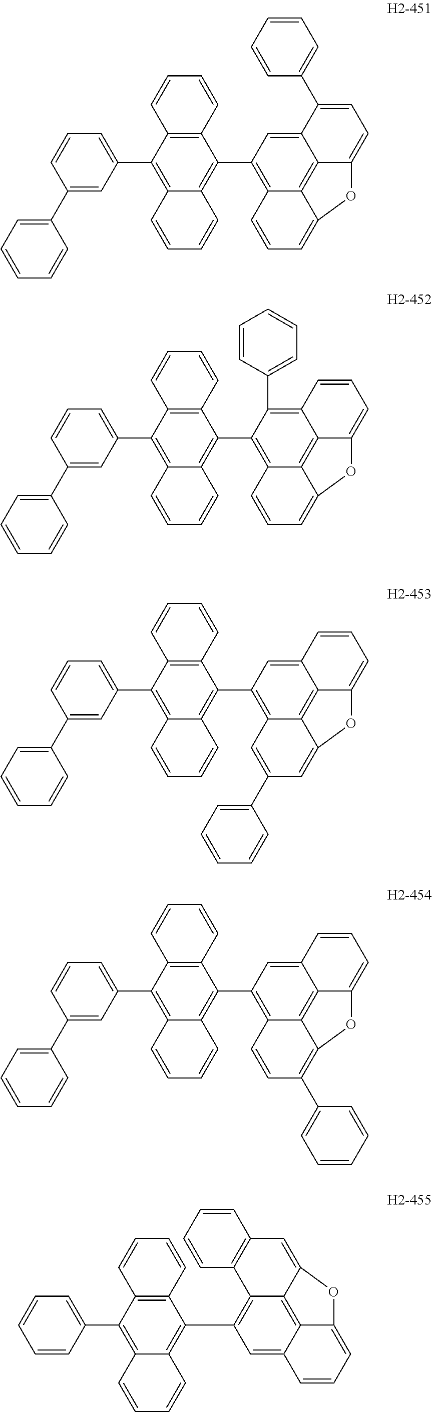

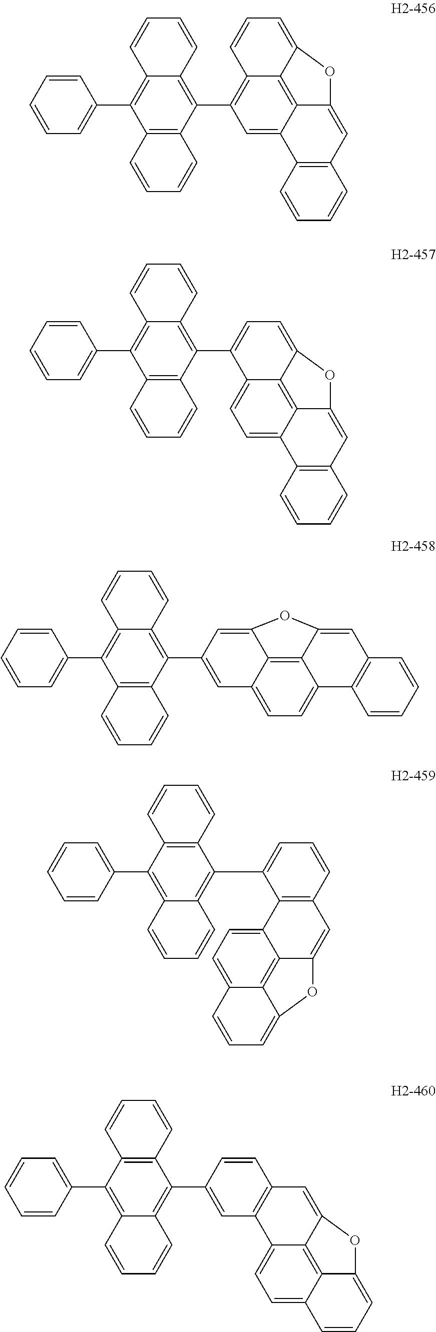

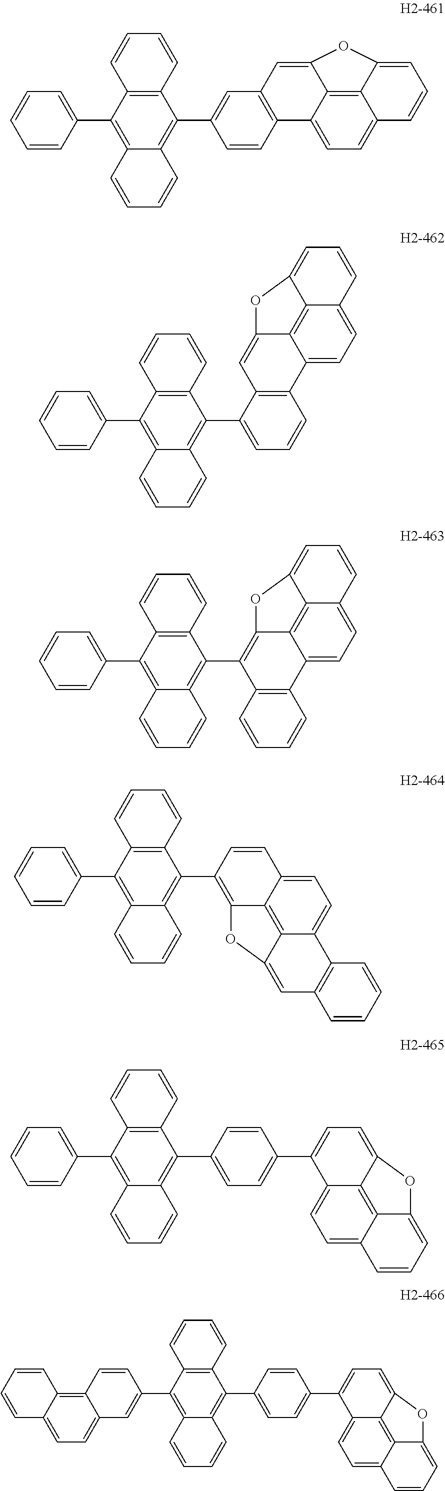

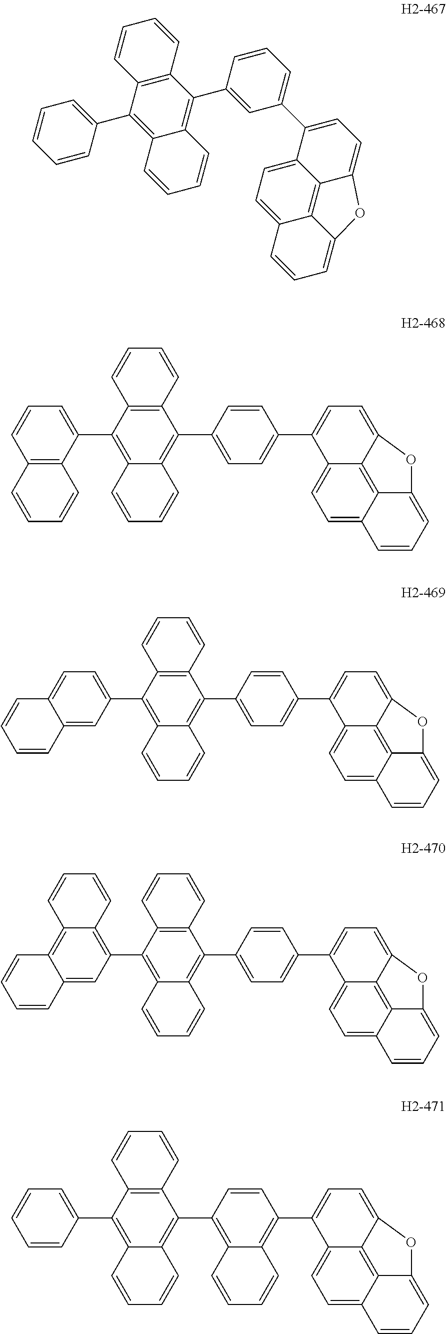 [SMILES: c1ccc(-c2c3ccccc3c(-c3cccc(-c4ccc5oc6cccc7ccc4c5c76)c3)c3ccccc23)cc1 ; c1ccc2c(-c3c4ccccc4c(-c4ccc(-c5ccc6oc7cccc8ccc5c6c87)cc4)c4ccccc34)cccc2c1 ; c1ccc2cc(-c3c4ccccc4c(-c4ccc(-c5ccc6oc7cccc8ccc5c6c87)cc4)c4ccccc34)ccc2c1 ; c1ccc2c(c1)cc(-c1c3ccccc3c(-c3ccc(-c4ccc5oc6cccc7ccc4c5c76)cc3)c3ccccc13)c1ccccc12 ; c1ccc(-c2c3ccccc3c(-c3ccc(-c4ccc5oc6cccc7ccc4c5c76)c4ccccc34)c3ccccc23)cc1]

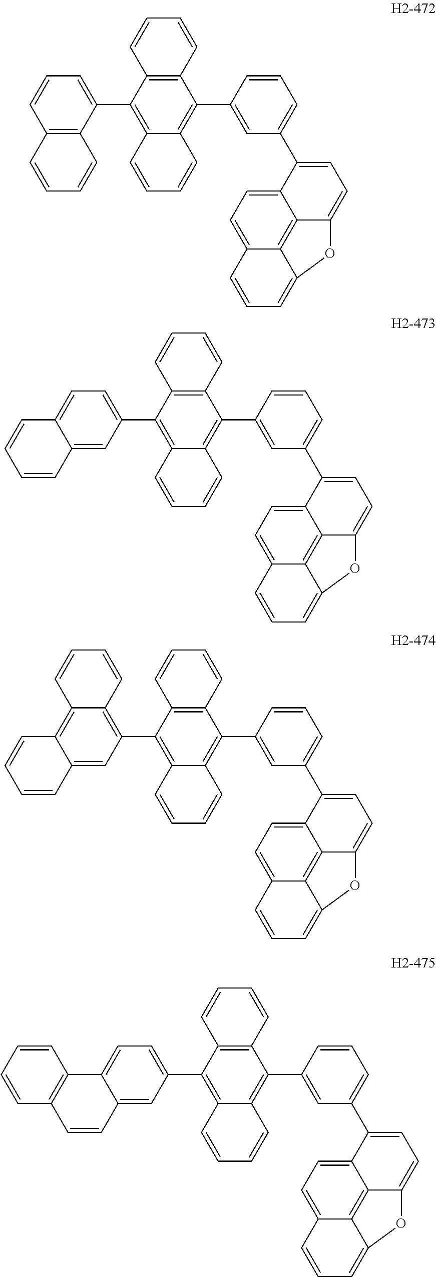 [SMILES: c1cc(-c2c3ccccc3c(-c3cccc4ccccc34)c3ccccc23)cc(-c2ccc3oc4cccc5ccc2c3c54)c1 ; c1cc(-c2c3ccccc3c(-c3ccc4ccccc4c3)c3ccccc23)cc(-c2ccc3oc4cccc5ccc2c3c54)c1 ; c1cc(-c2c3ccccc3c(-c3cc4ccccc4c4ccccc34)c3ccccc23)cc(-c2ccc3oc4cccc5ccc2c3c54)c1 ; c1cc(-c2c3ccccc3c(-c3ccc4c(ccc5ccccc54)c3)c3ccccc23)cc(-c2ccc3oc4cccc5ccc2c3c54)c1]

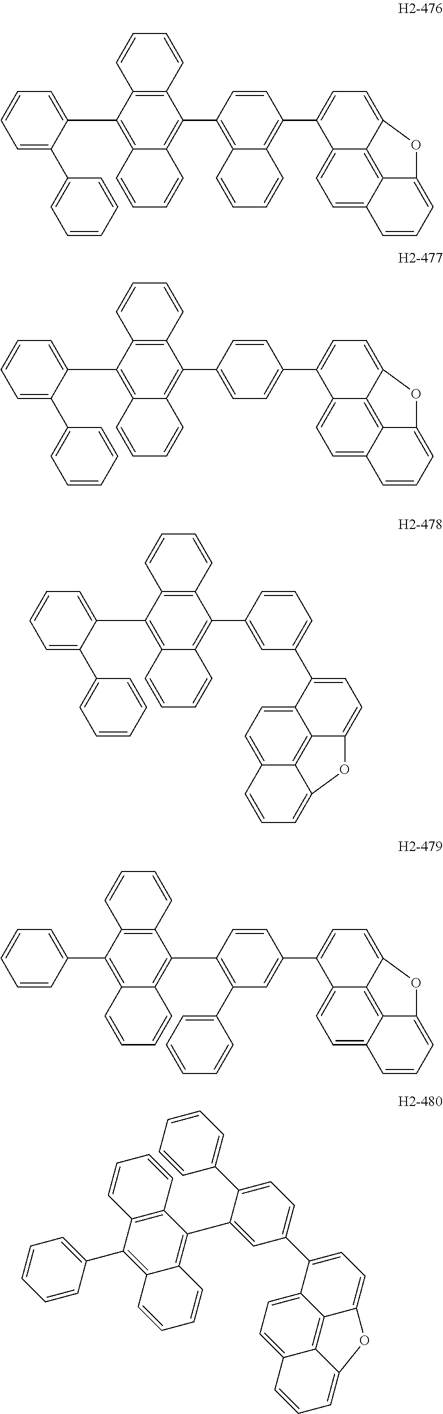

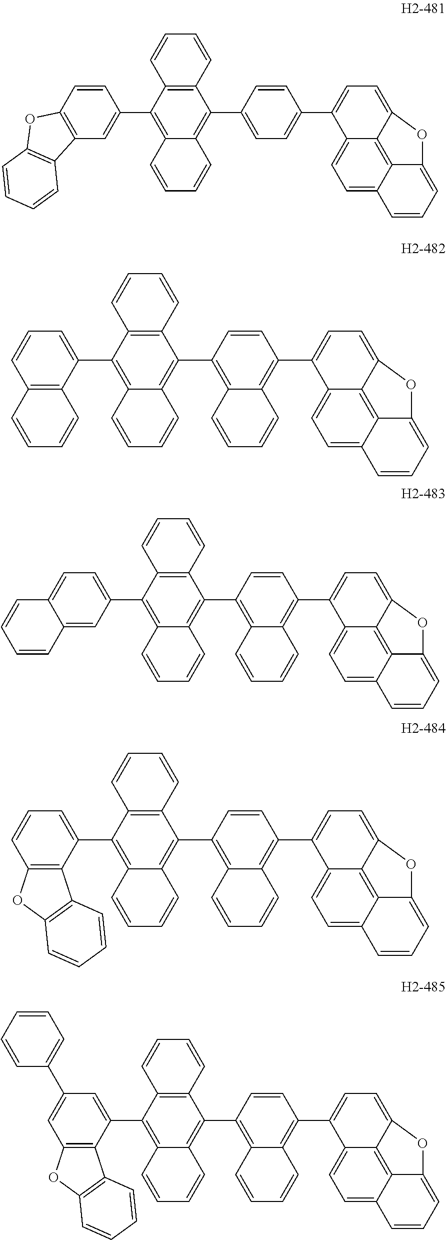 [SMILES: c1ccc2c(c1)oc1ccc(-c3c4ccccc4c(-c4ccc(-c5ccc6oc7cccc8ccc5c6c87)cc4)c4ccccc34)cc12 ; c1ccc2c(-c3c4ccccc4c(-c4ccc(-c5ccc6oc7cccc8ccc5c6c87)c5ccccc45)c4ccccc34)cccc2c1 ; c1ccc2cc(-c3c4ccccc4c(-c4ccc(-c5ccc6oc7cccc8ccc5c6c87)c5ccccc45)c4ccccc34)ccc2c1 ; c1ccc2c(c1)oc1cccc(-c3c4ccccc4c(-c4ccc(-c5ccc6oc7cccc8ccc5c6c87)c5ccccc45)c4ccccc34)c12 ; c1ccc(-c2cc(-c3c4ccccc4c(-c4ccc(-c5ccc6oc7cccc8ccc5c6c87)c5ccccc45)c4ccccc34)c3c(c2)oc2ccccc23)cc1]

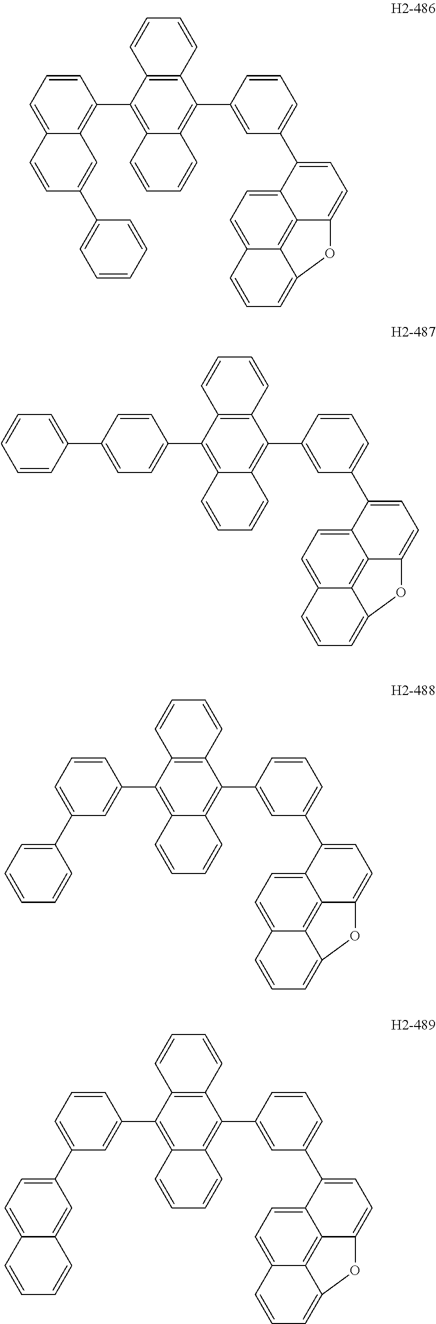 [SMILES: c1ccc(-c2ccc3cccc(-c4c5ccccc5c(-c5cccc(-c6ccc7oc8cccc9ccc6c7c98)c5)c5ccccc45)c3c2)cc1 ; c1ccc(-c2ccc(-c3c4ccccc4c(-c4cccc(-c5ccc6oc7cccc8ccc5c6c87)c4)c4ccccc34)cc2)cc1 ; c1ccc(-c2cccc(-c3c4ccccc4c(-c4cccc(-c5ccc6oc7cccc8ccc5c6c87)c4)c4ccccc34)c2)cc1 ; c1cc(-c2ccc3ccccc3c2)cc(-c2c3ccccc3c(-c3cccc(-c4ccc5oc6cccc7ccc4c5c76)c3)c3ccccc23)c1]

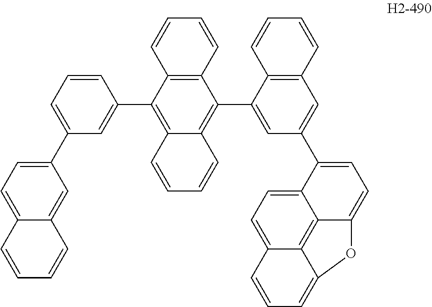 [SMILES: c1cc(-c2ccc3ccccc3c2)cc(-c2c3ccccc3c(-c3cc(-c4ccc5oc6cccc7ccc4c5c76)cc4ccccc34)c3ccccc23)c1]

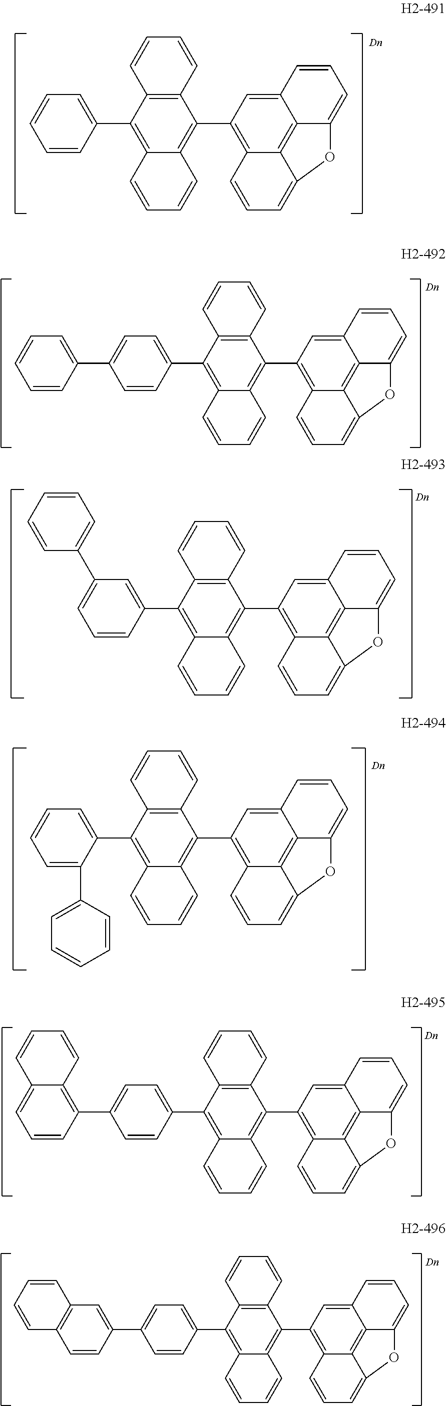

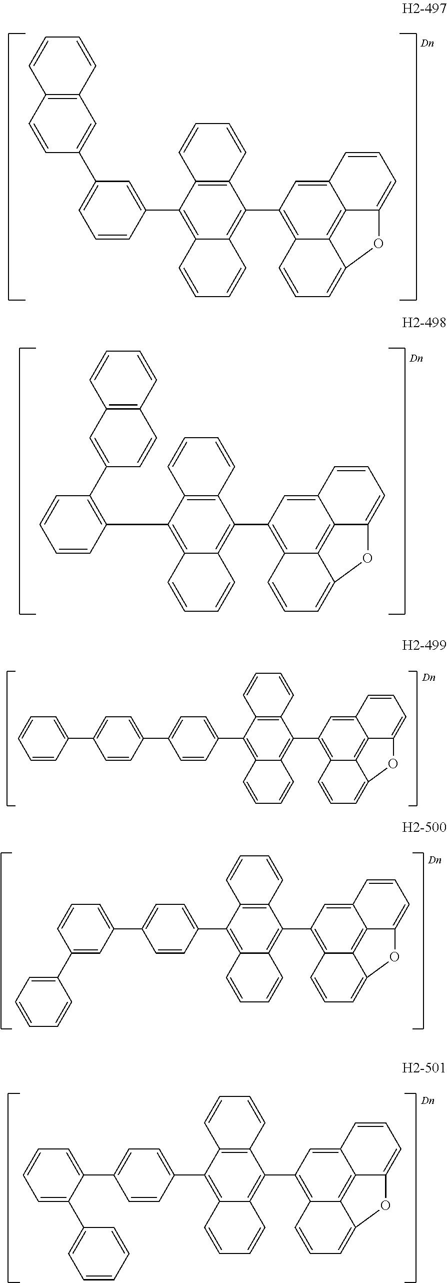

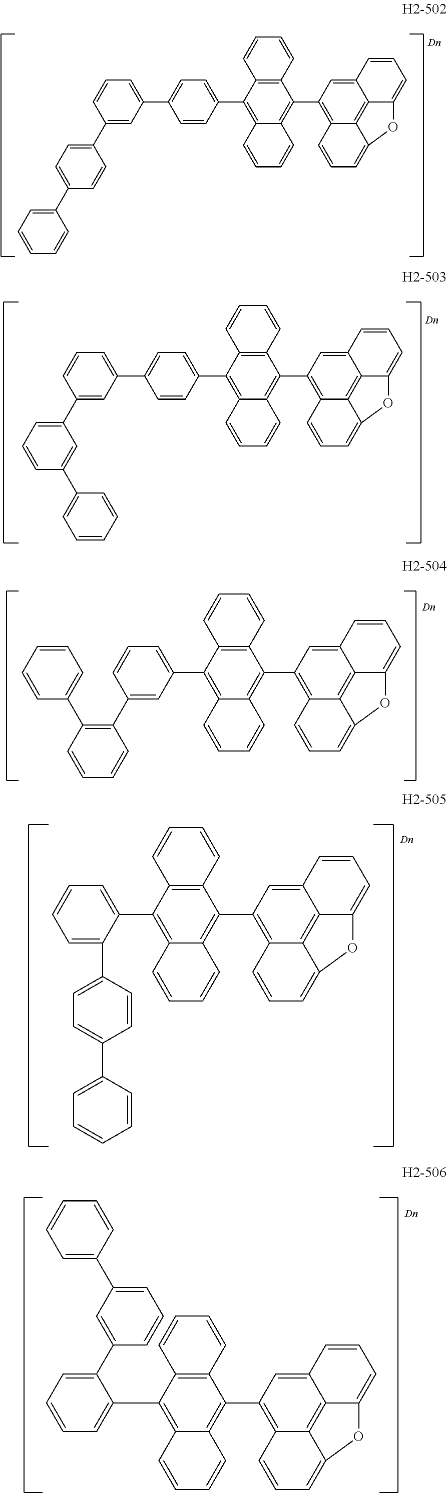

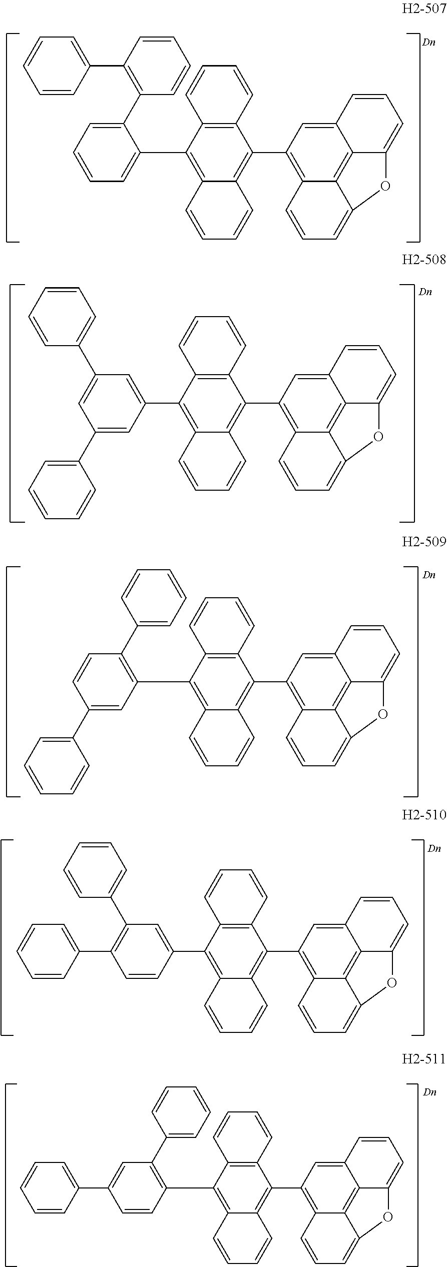

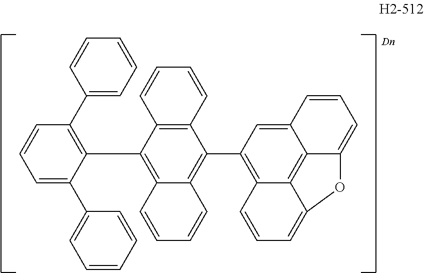

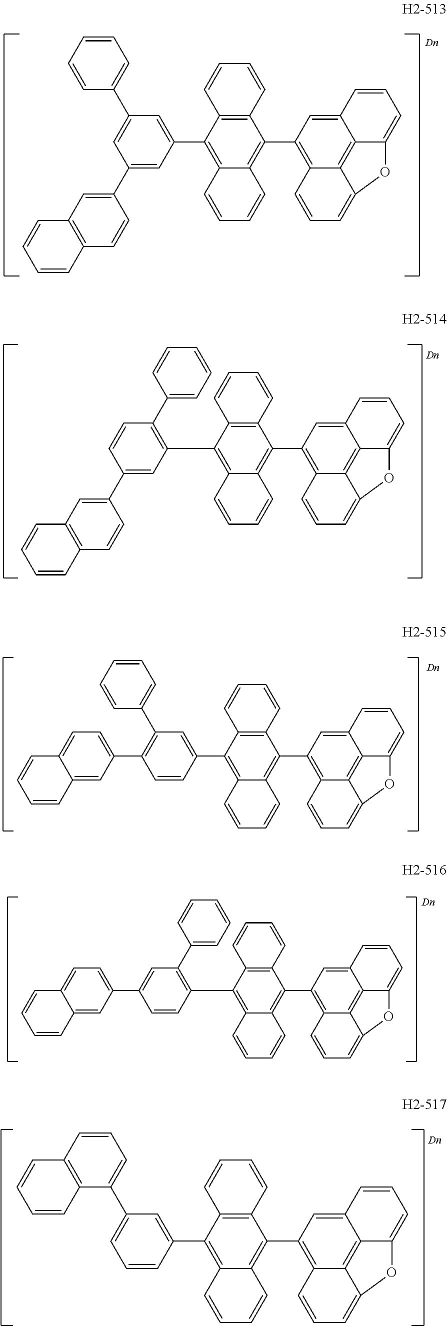

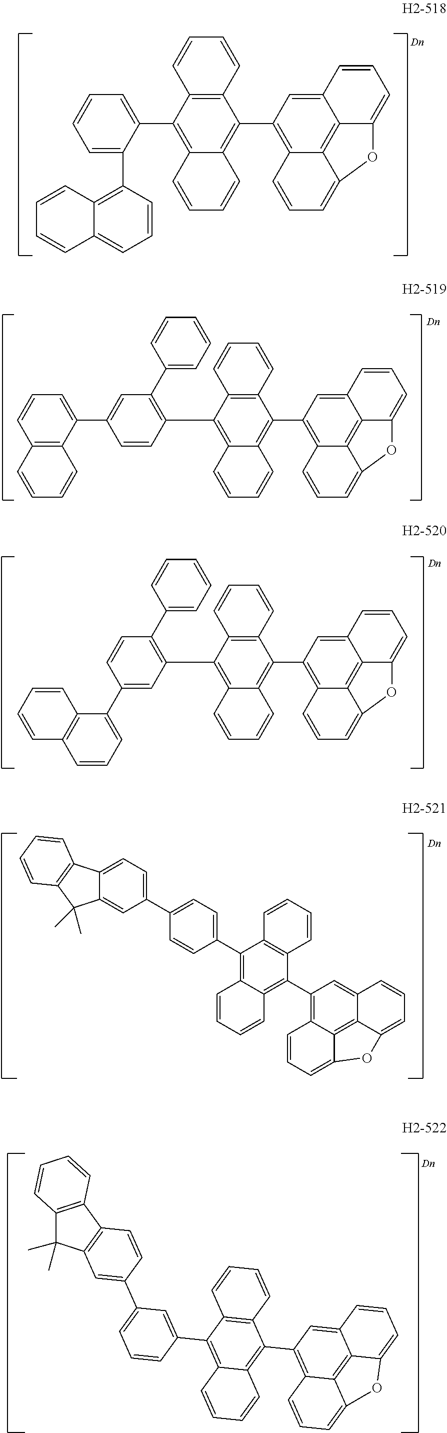

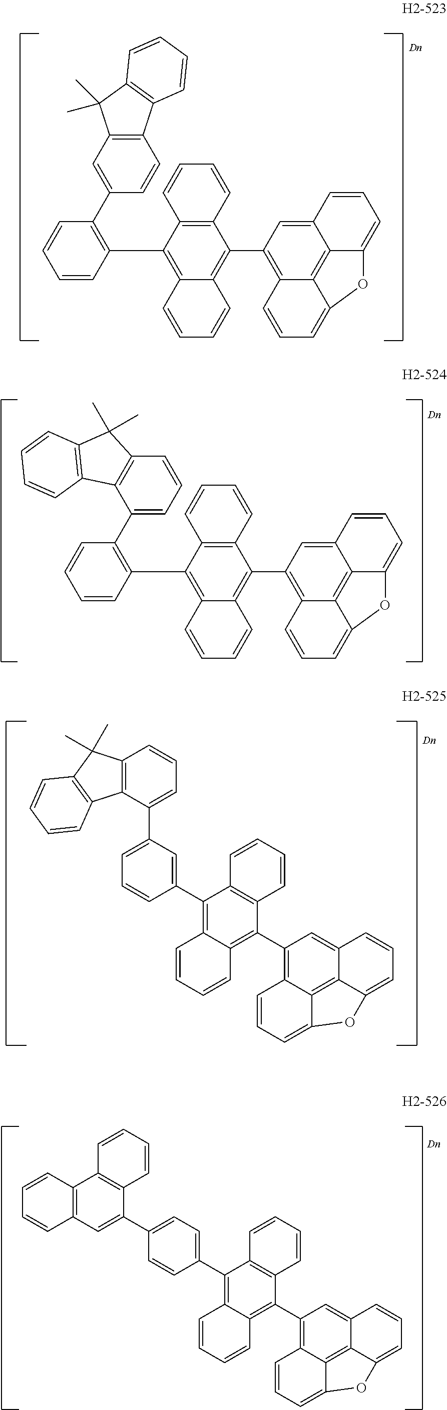

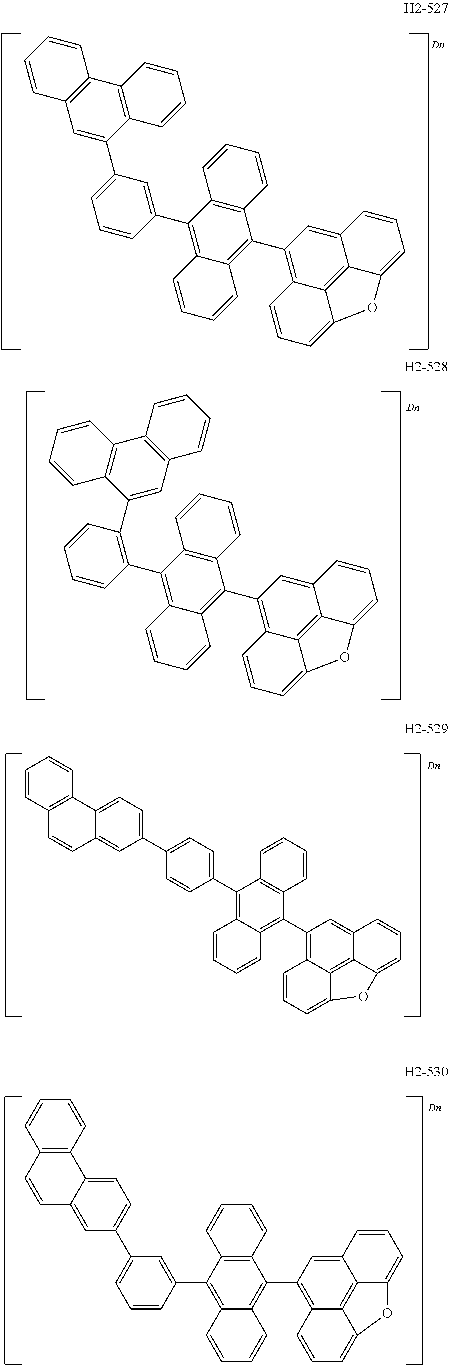

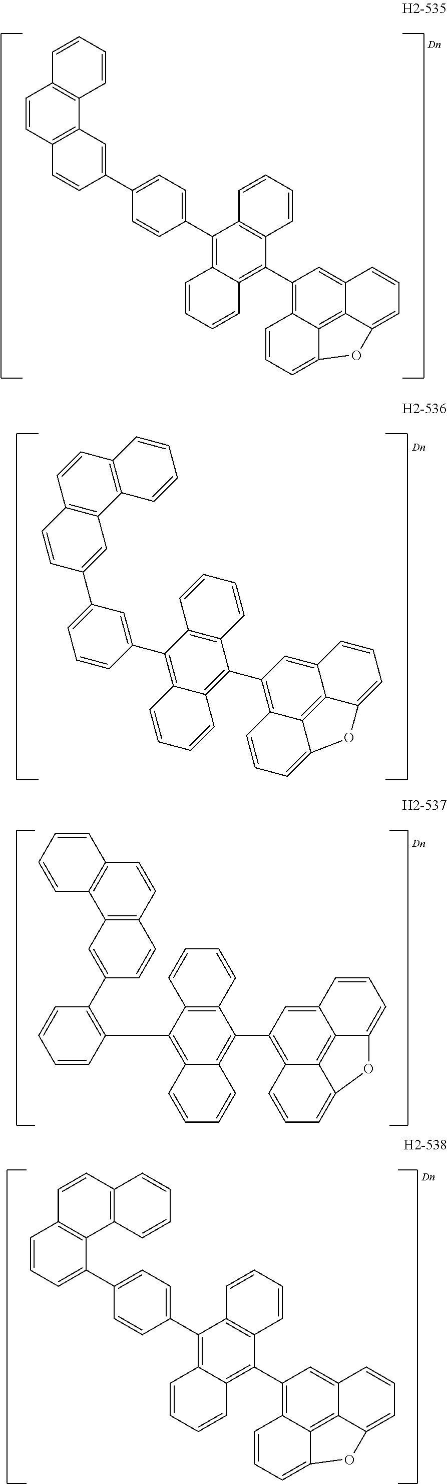

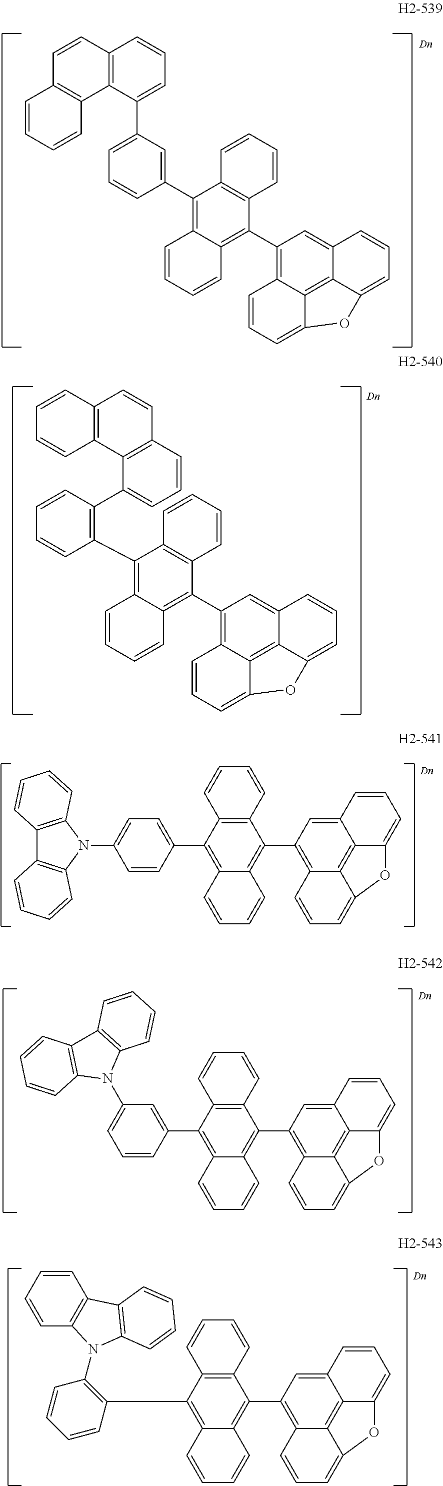

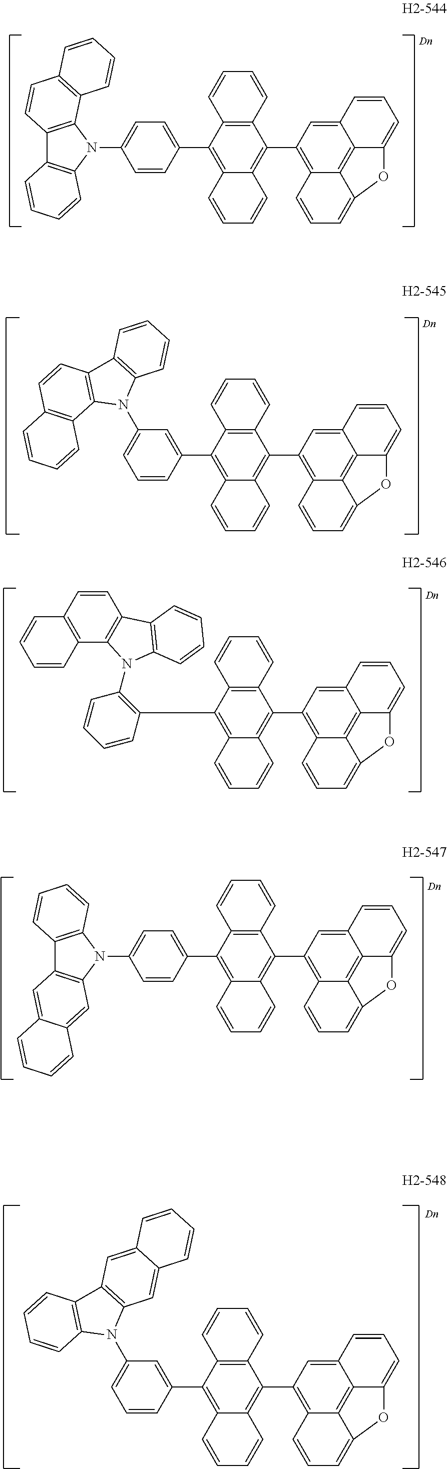

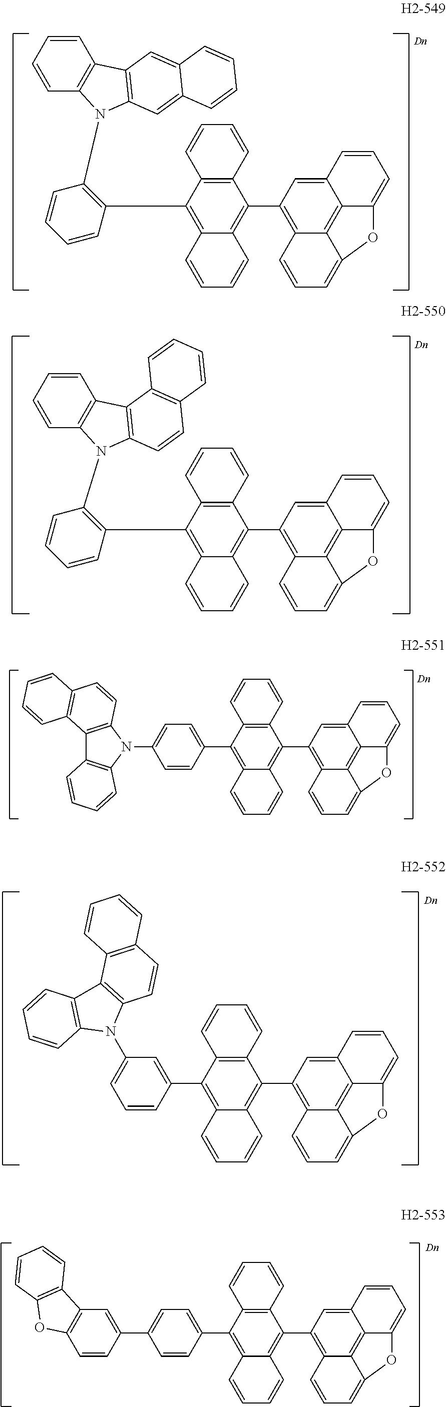

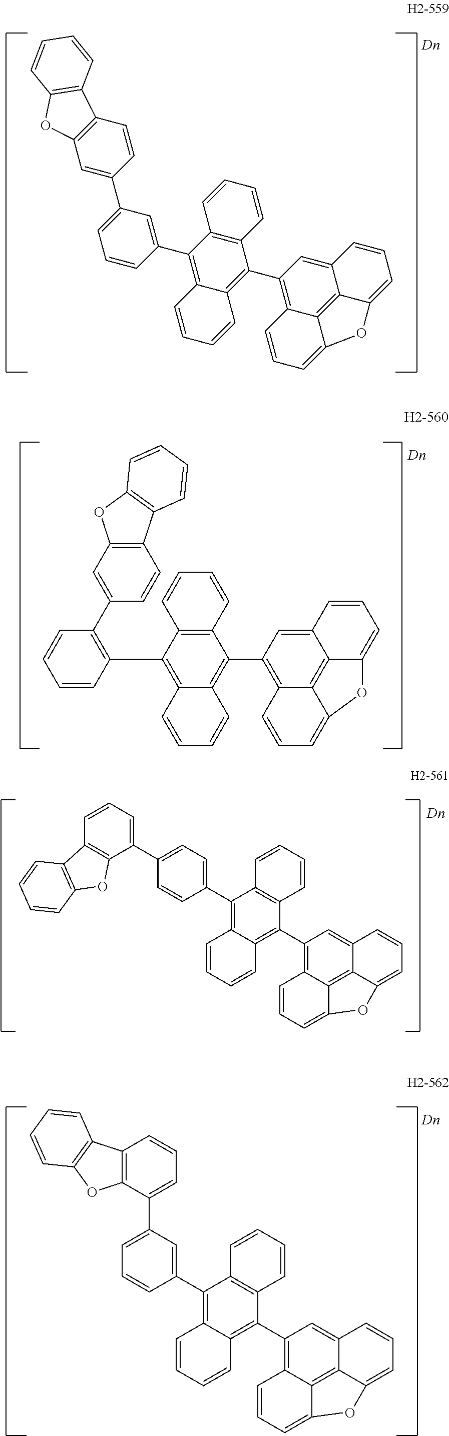

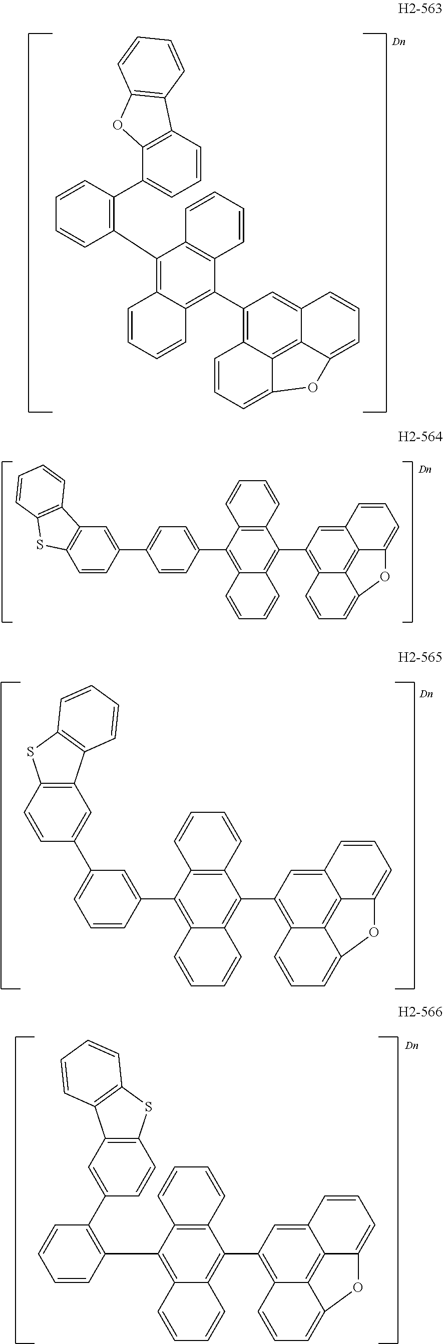

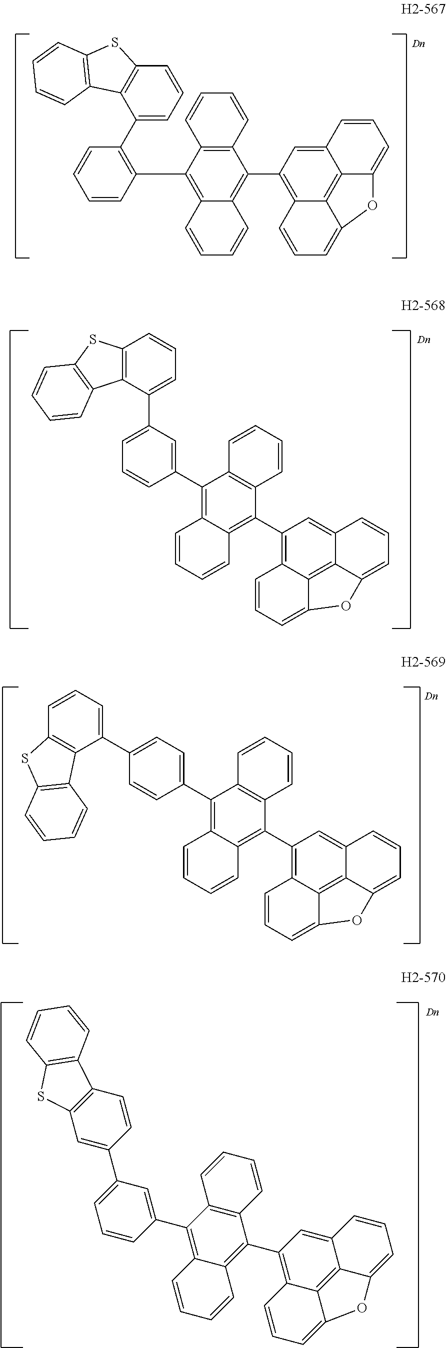

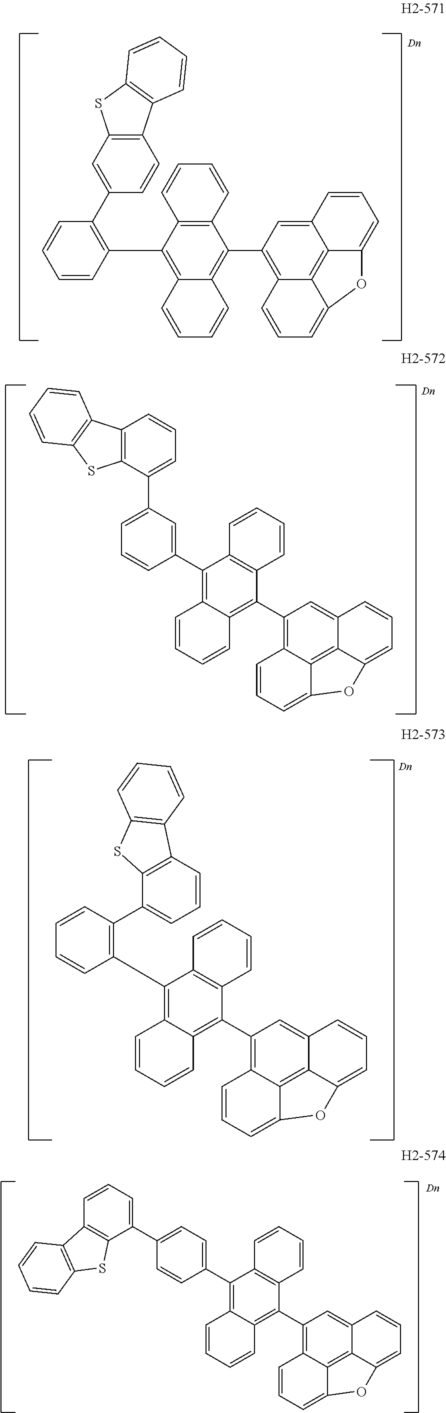

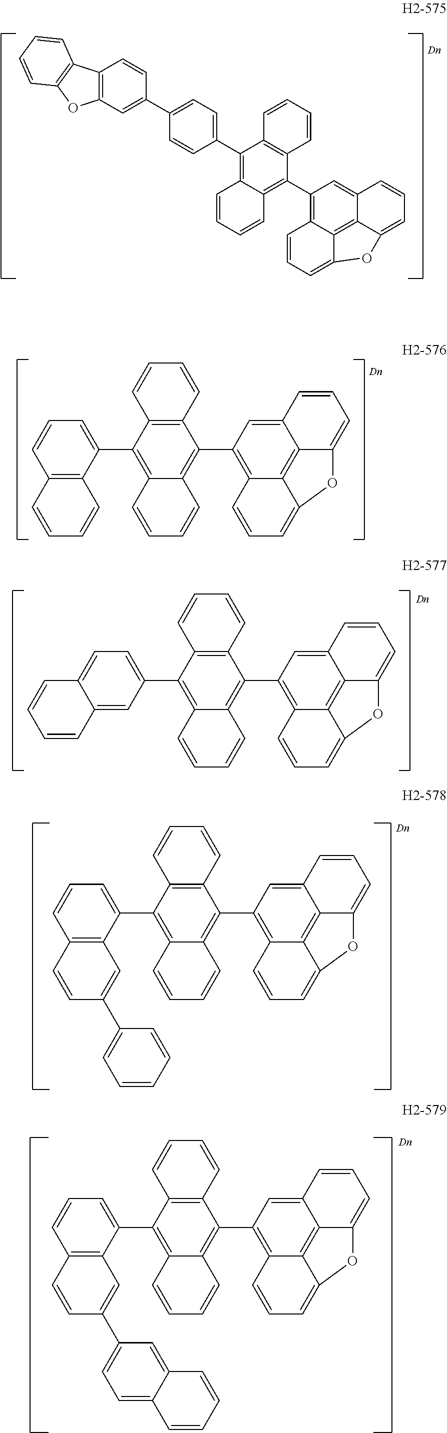

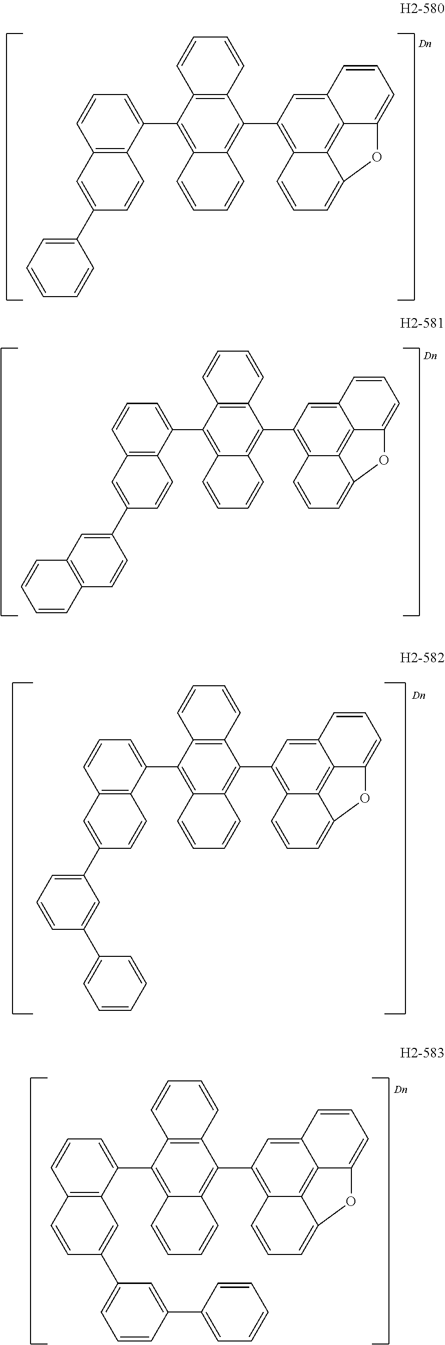

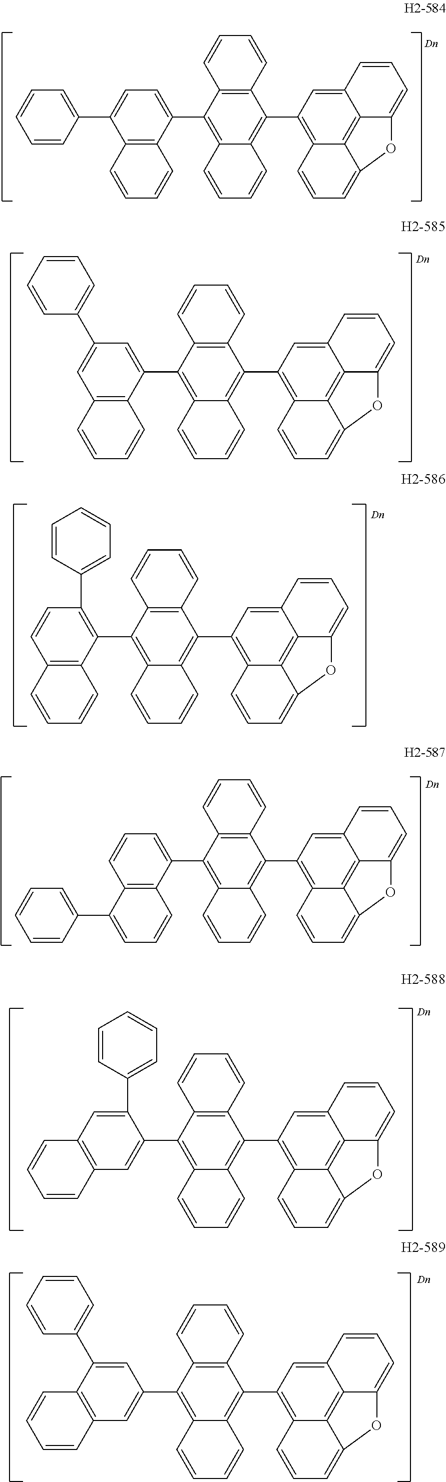

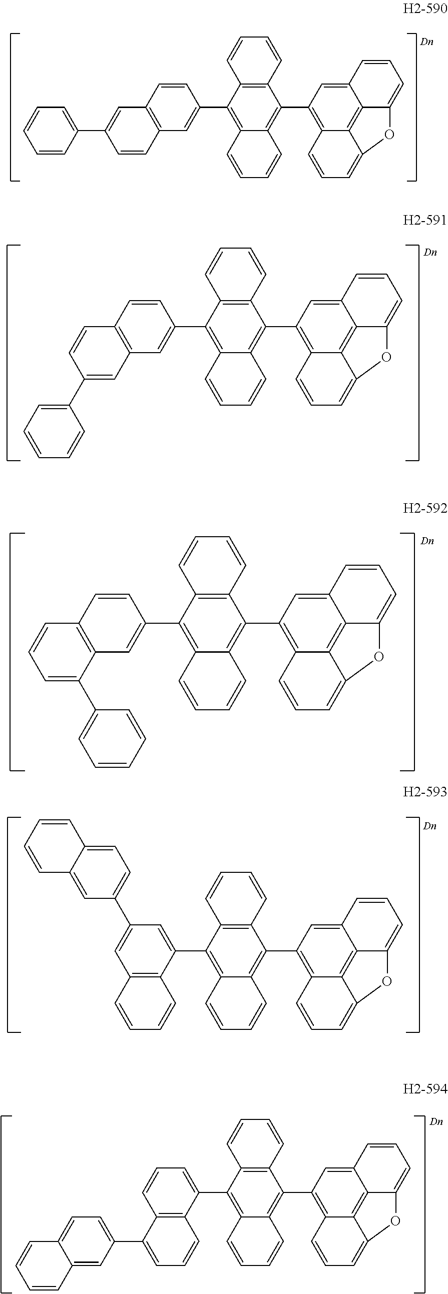

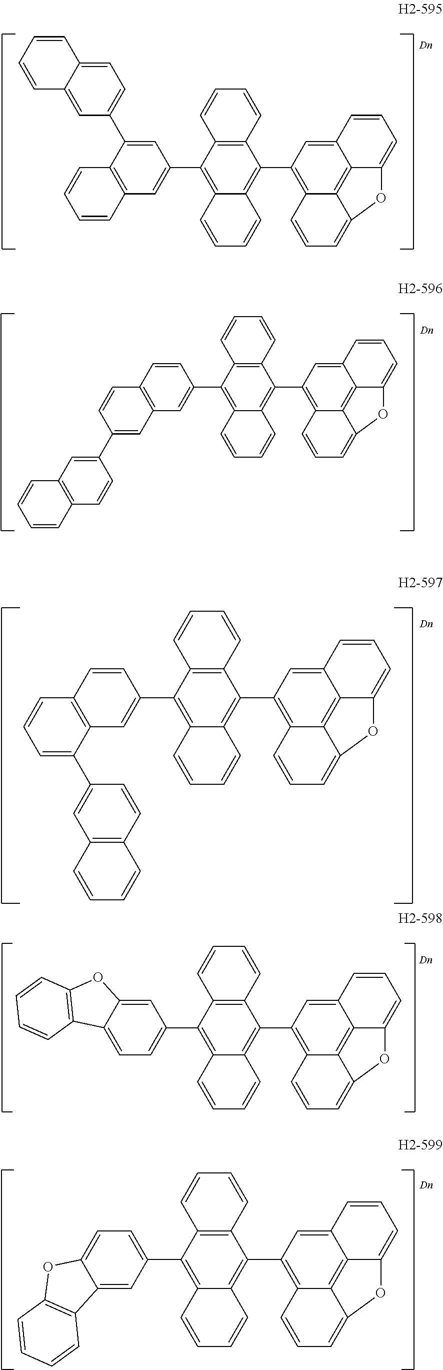

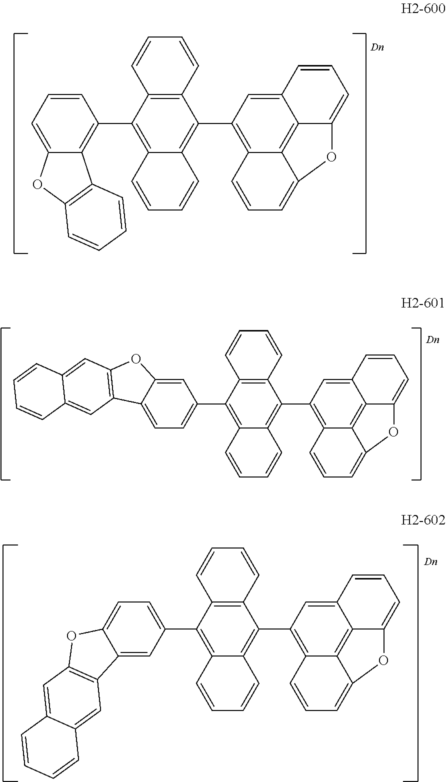

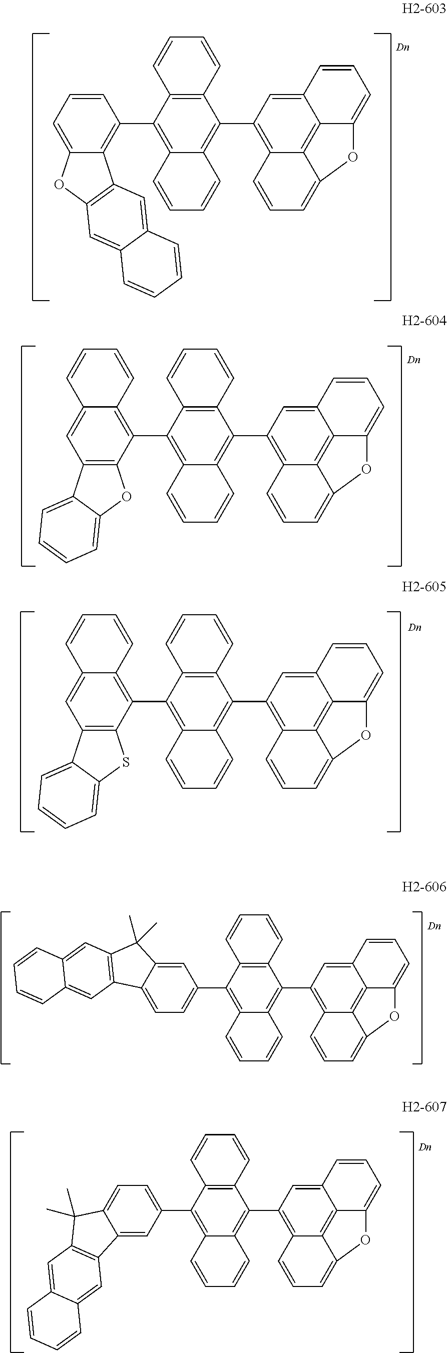

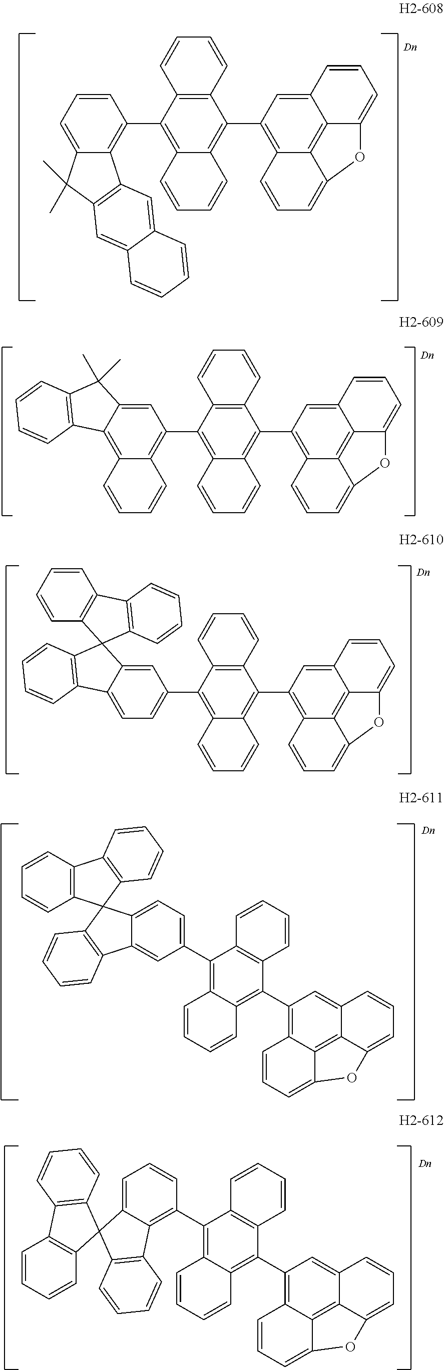

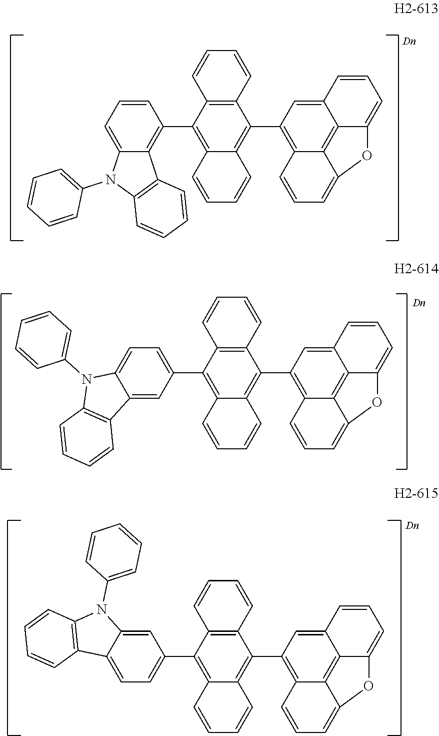

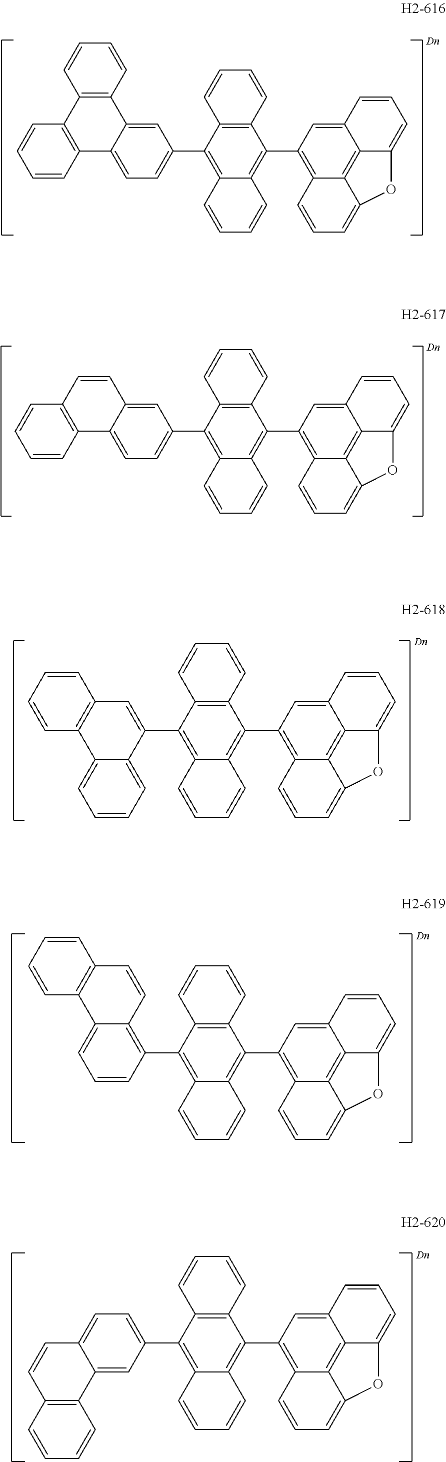

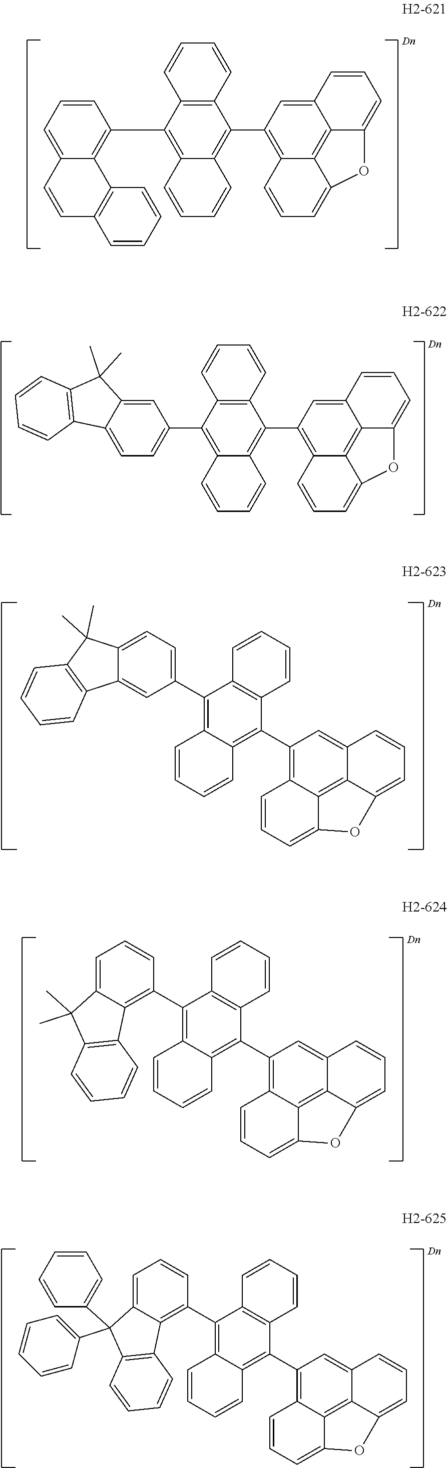

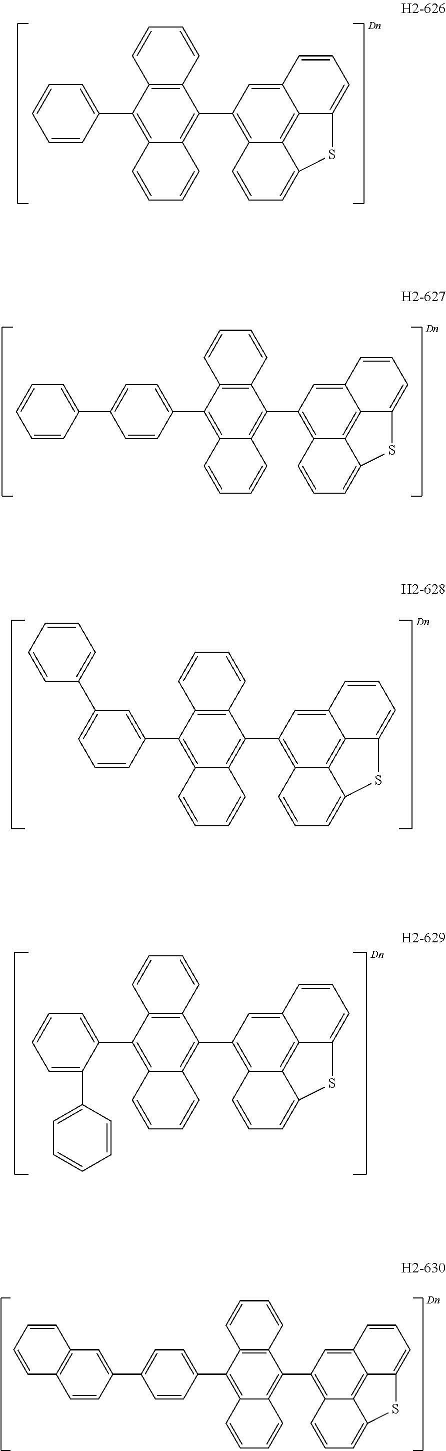

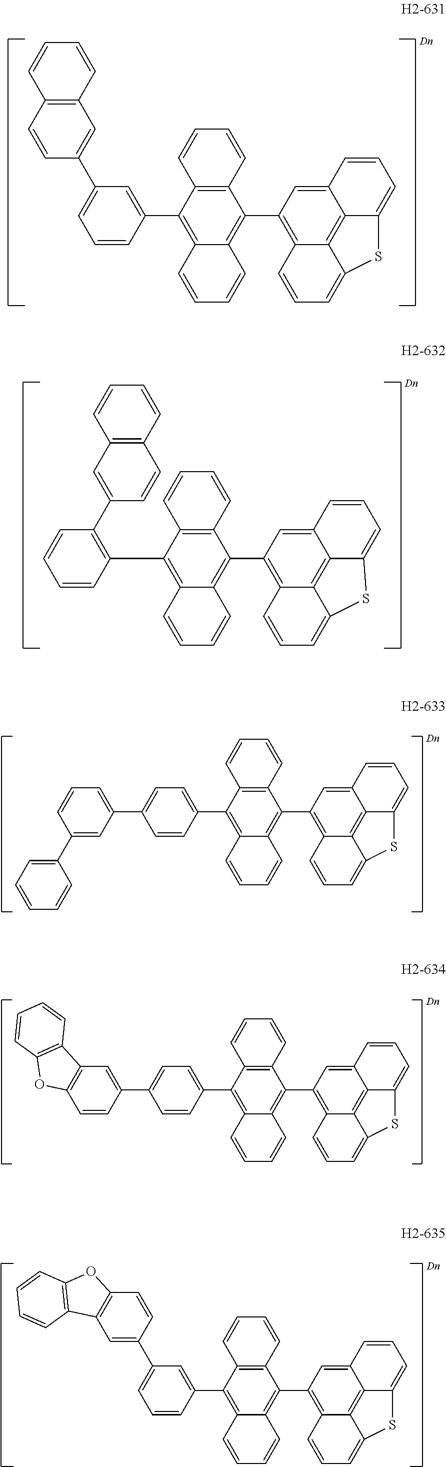

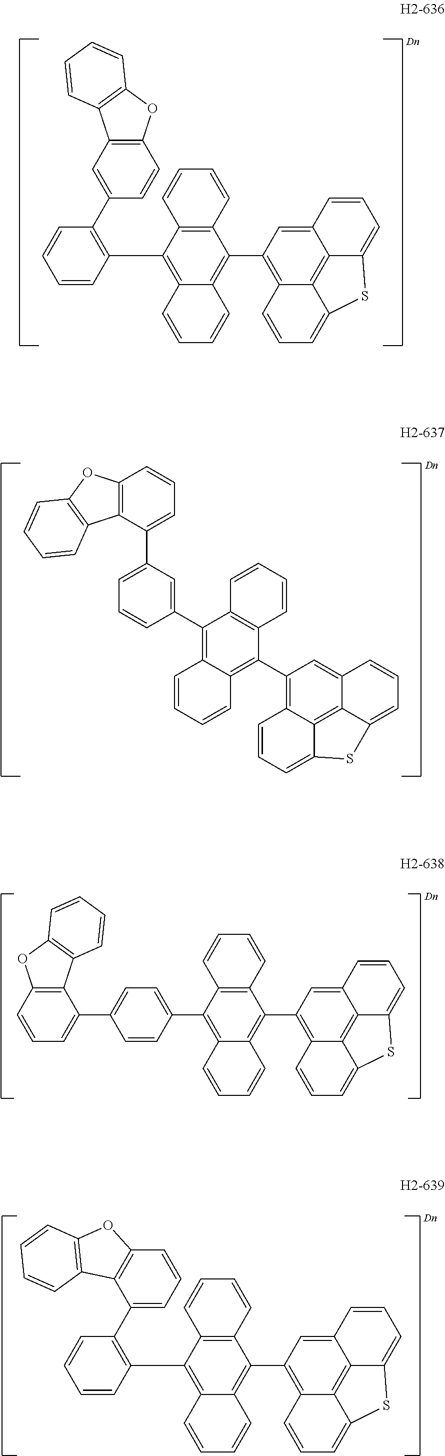

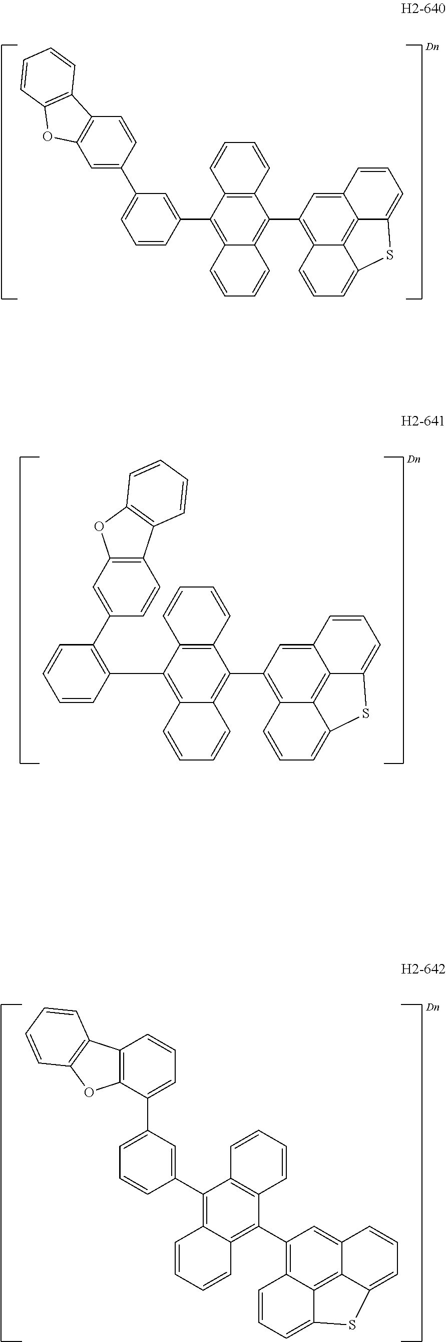

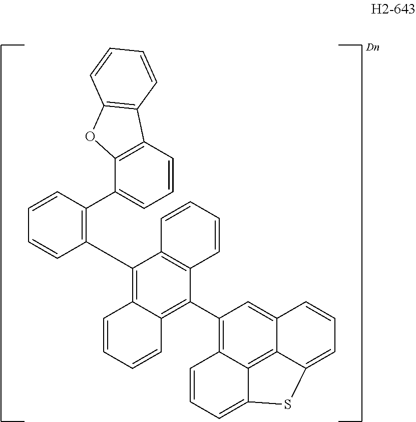

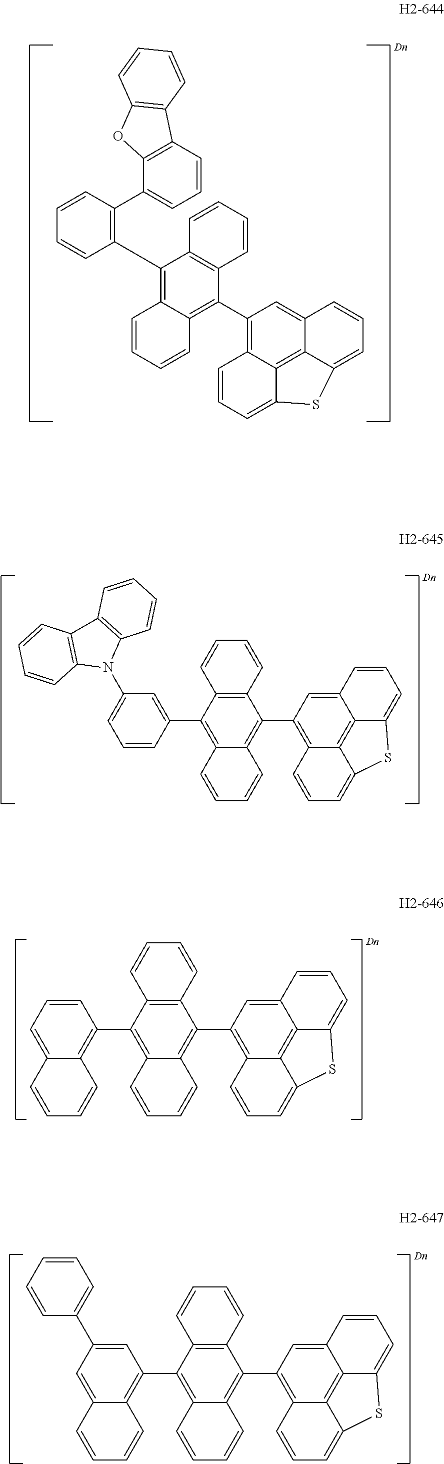

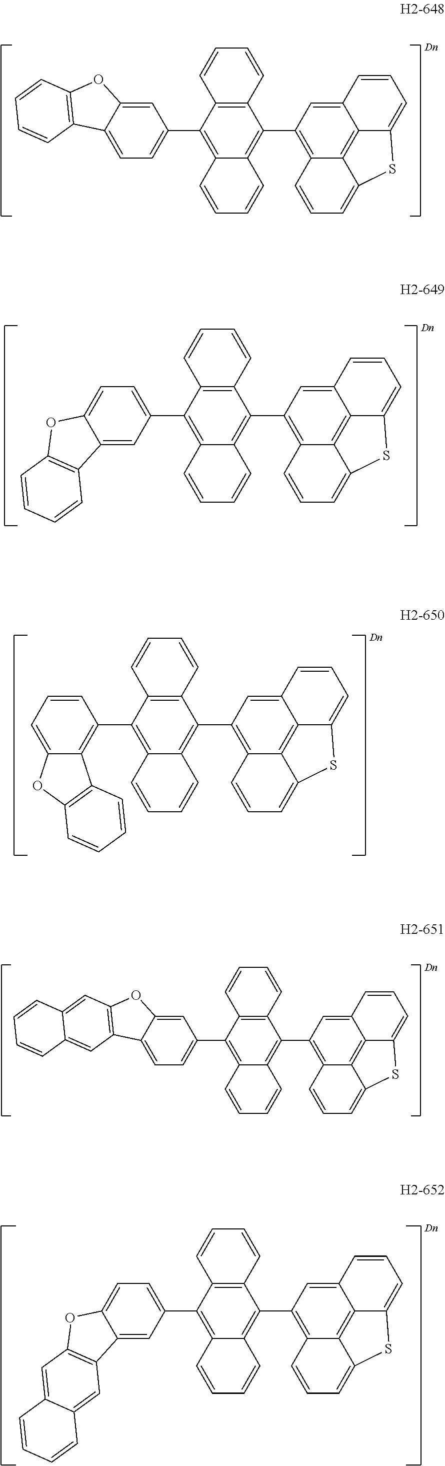

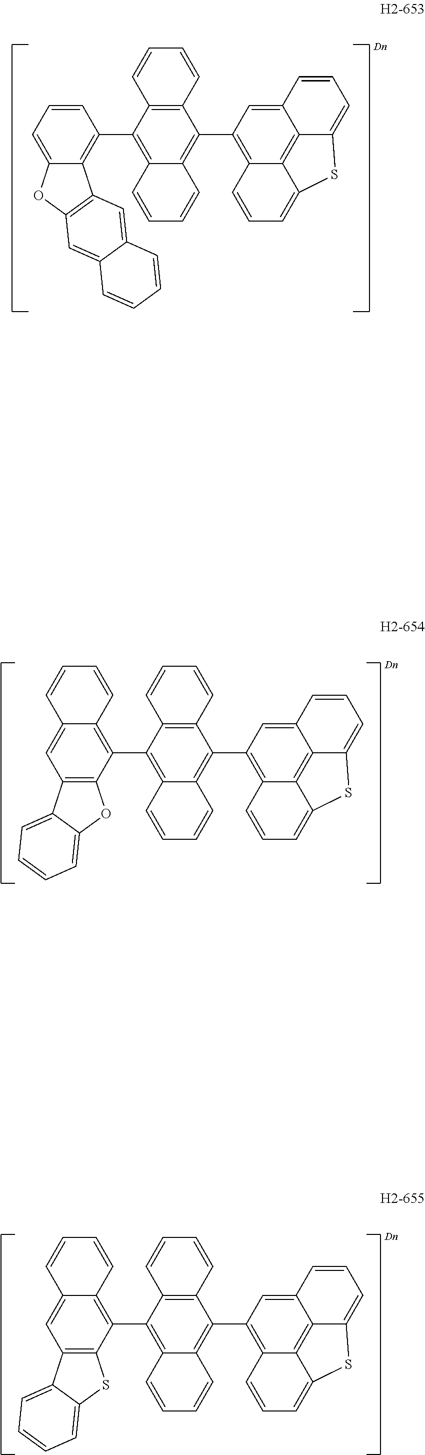

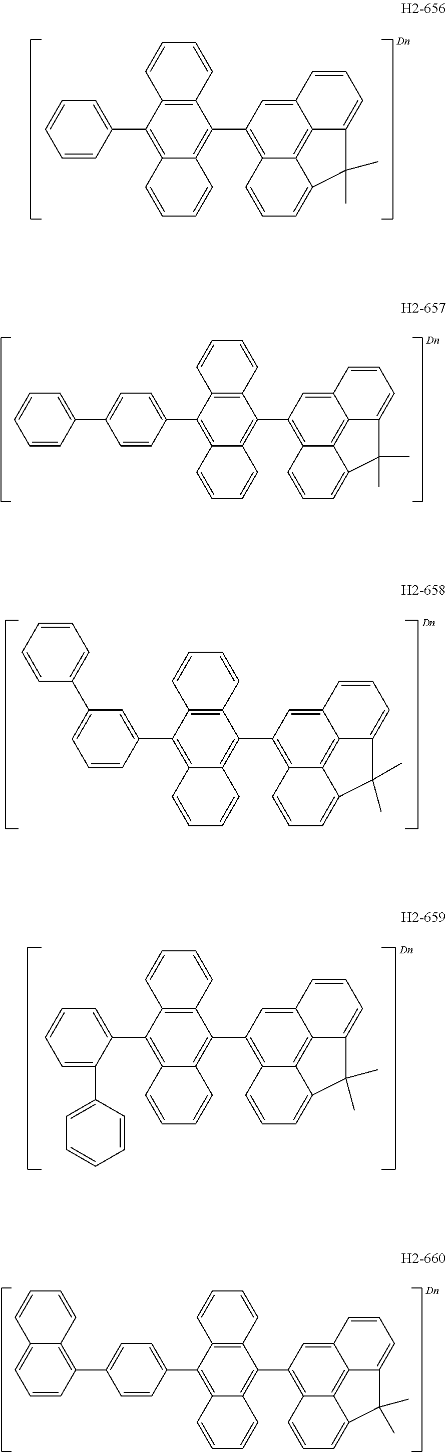

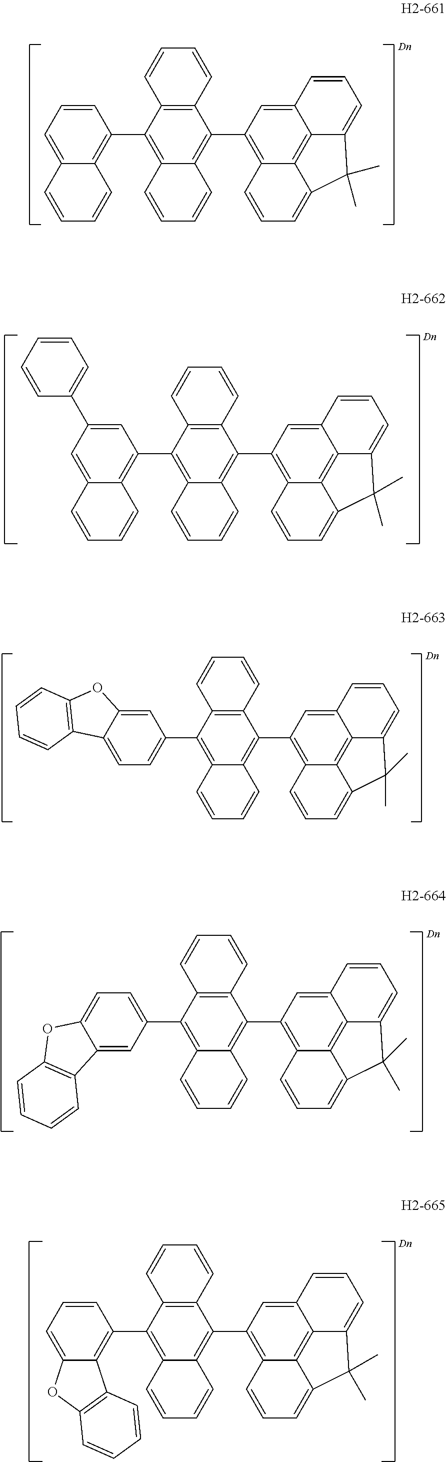

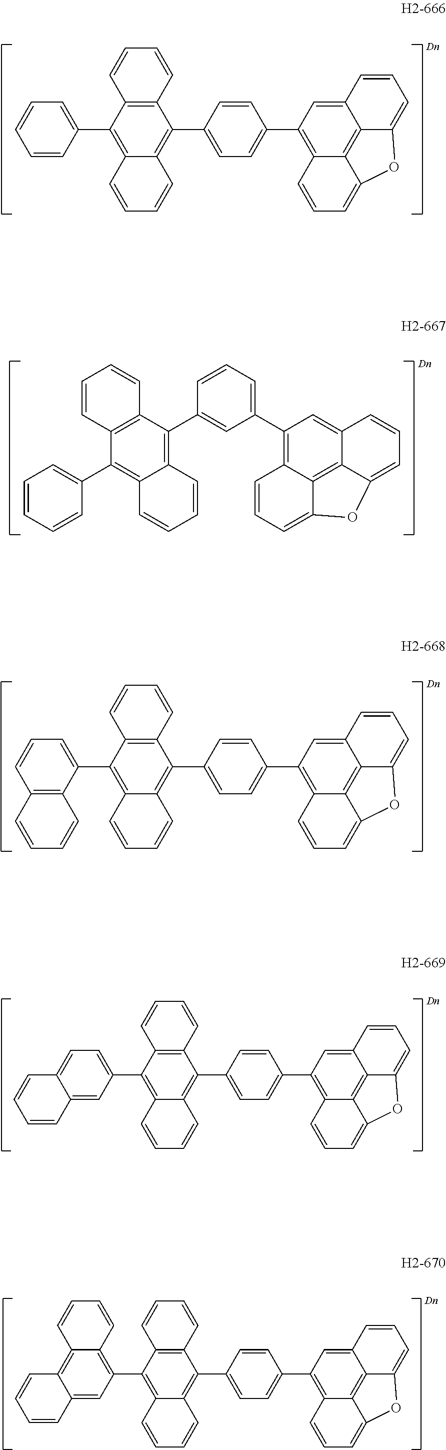

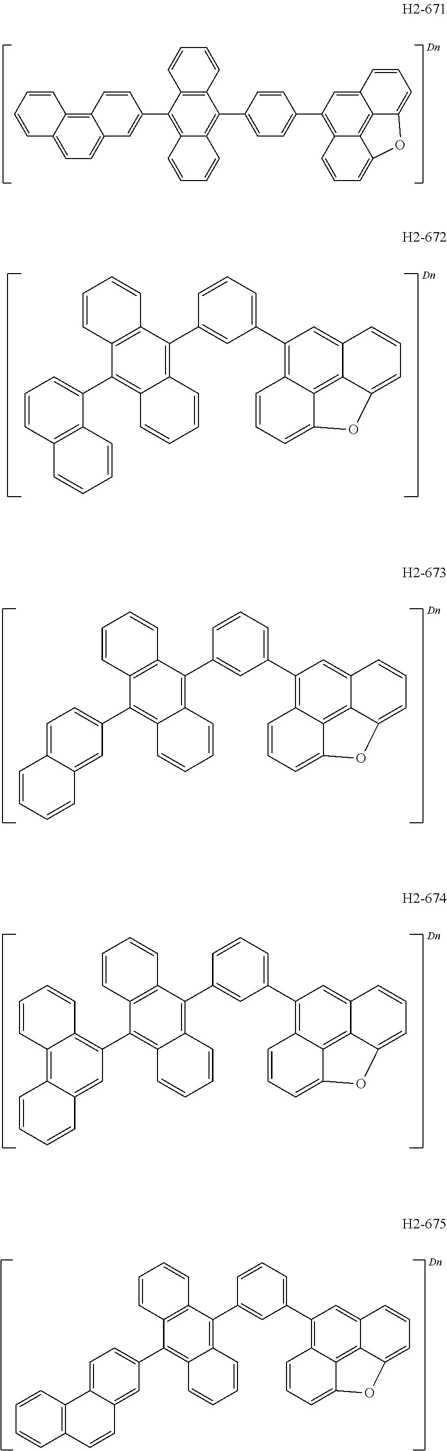

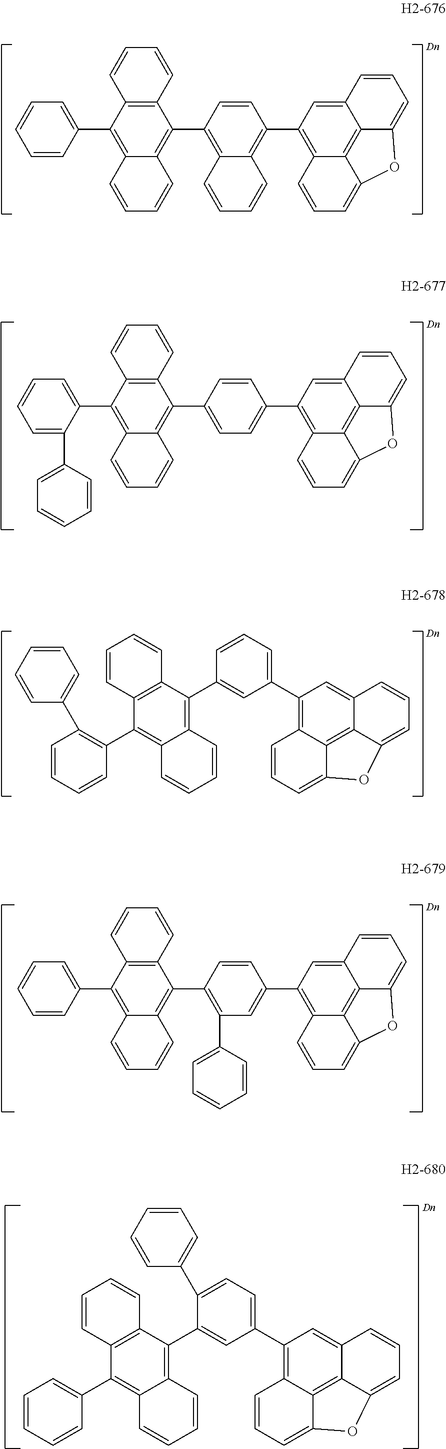

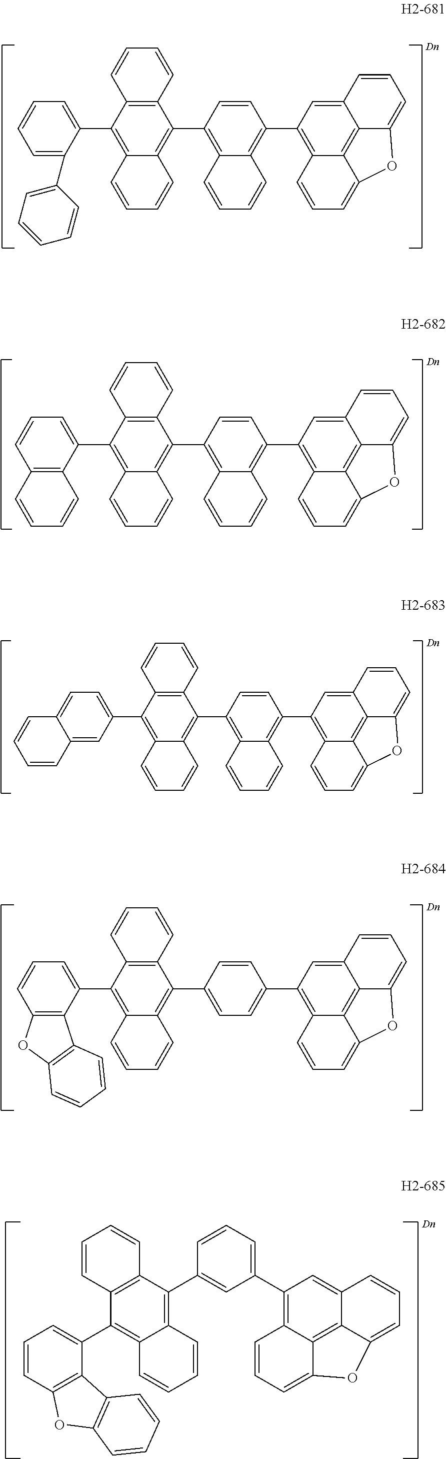

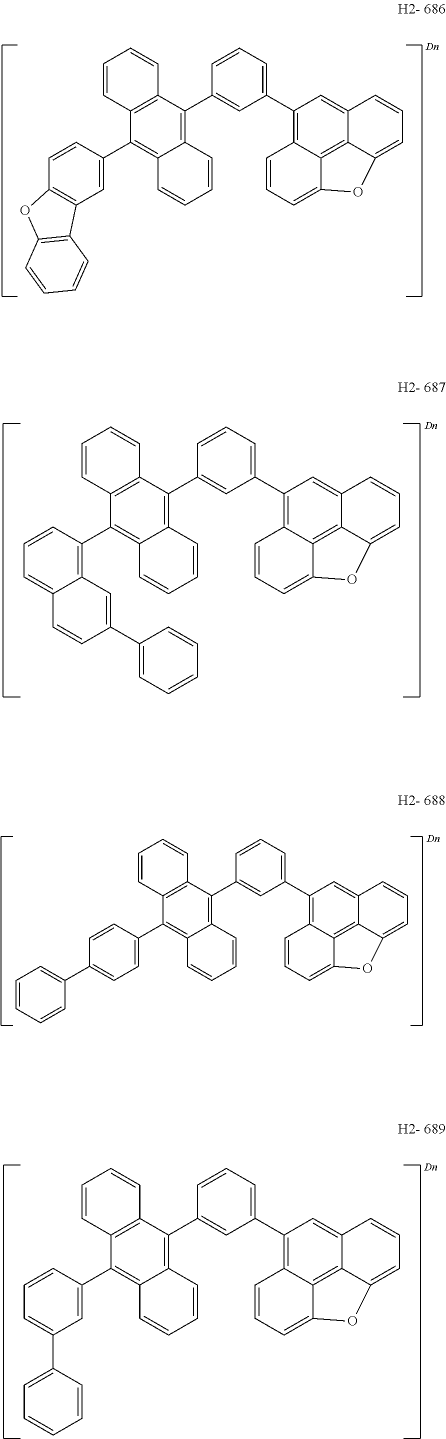

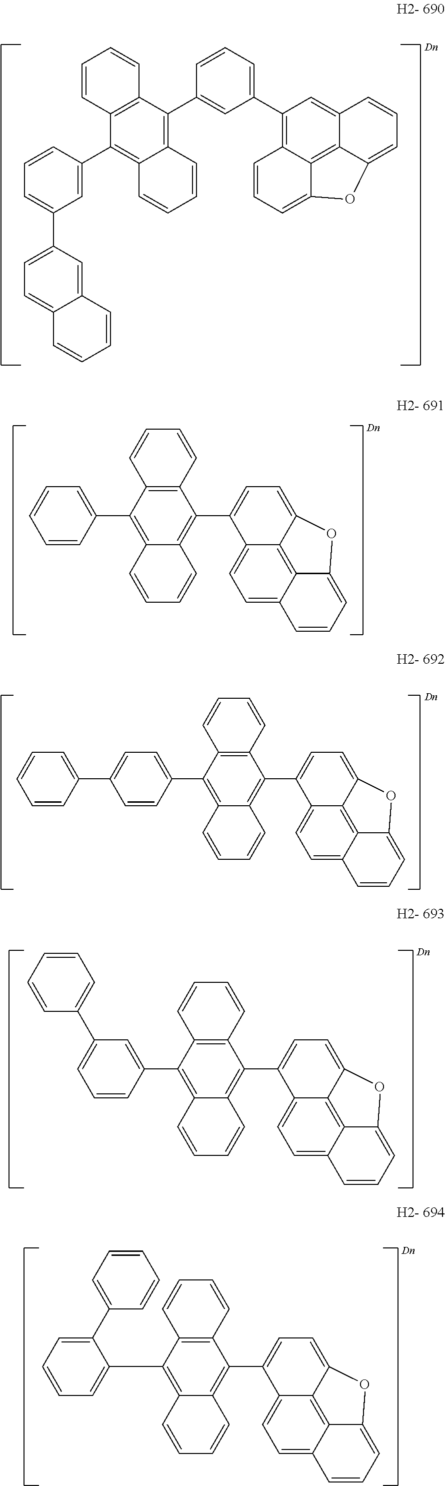

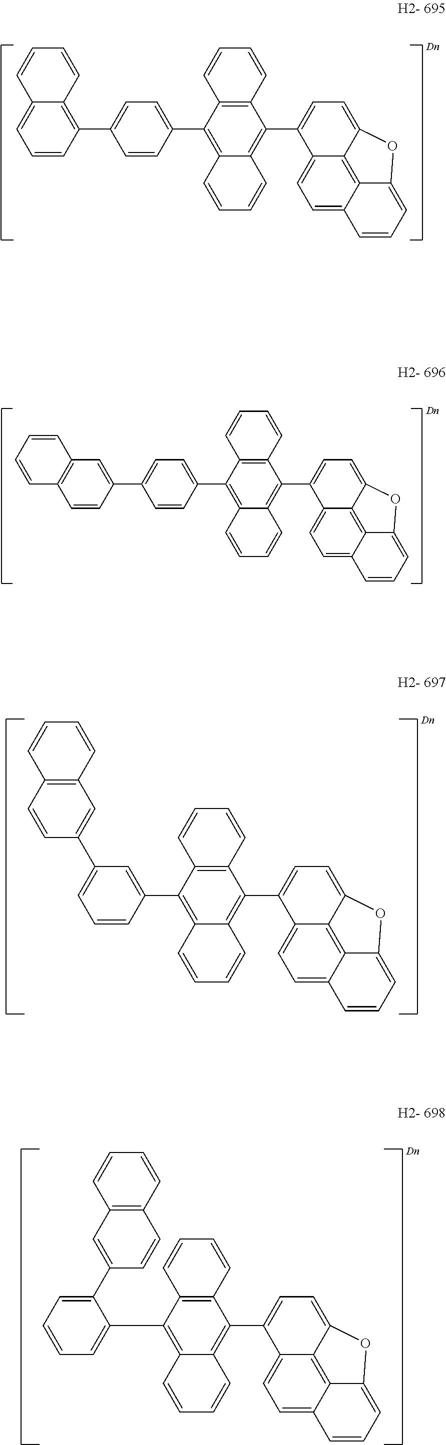

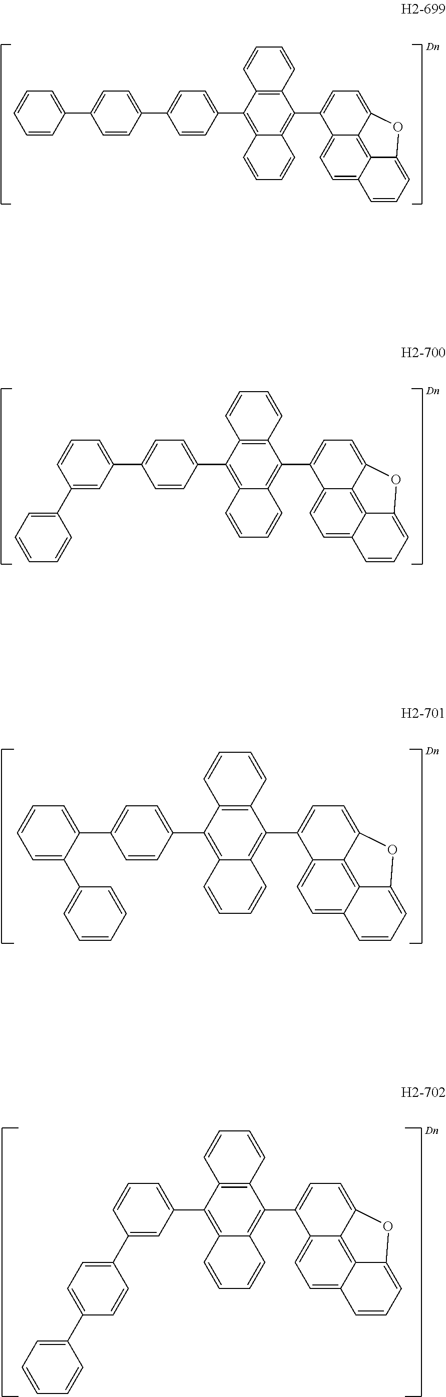

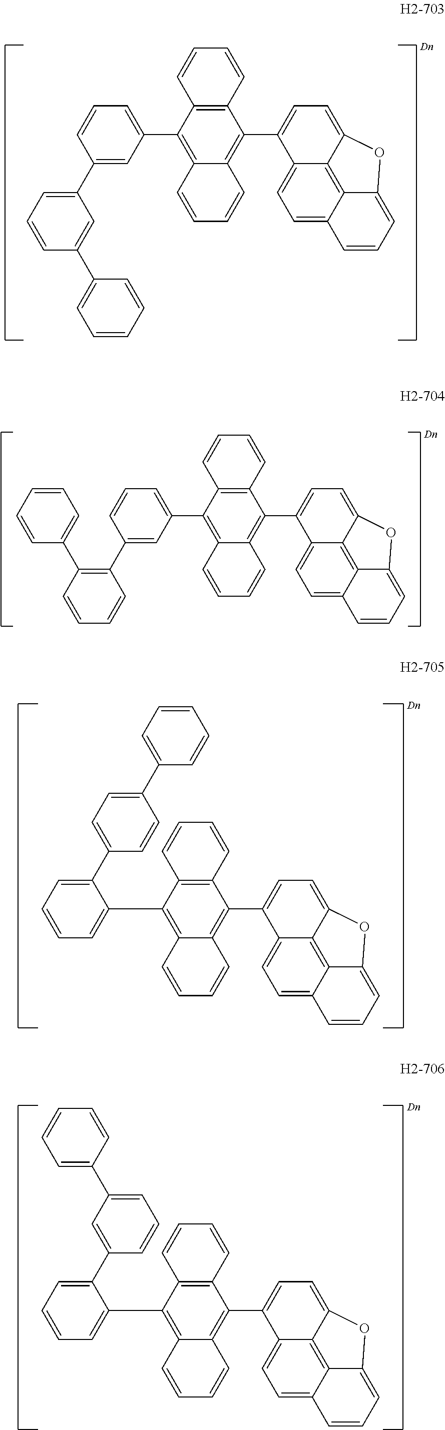

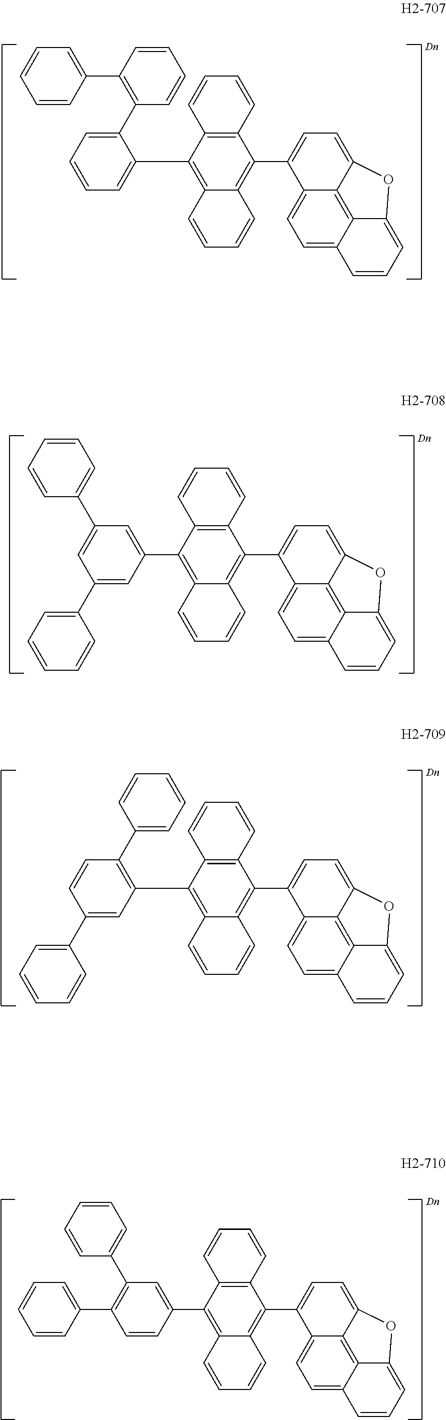

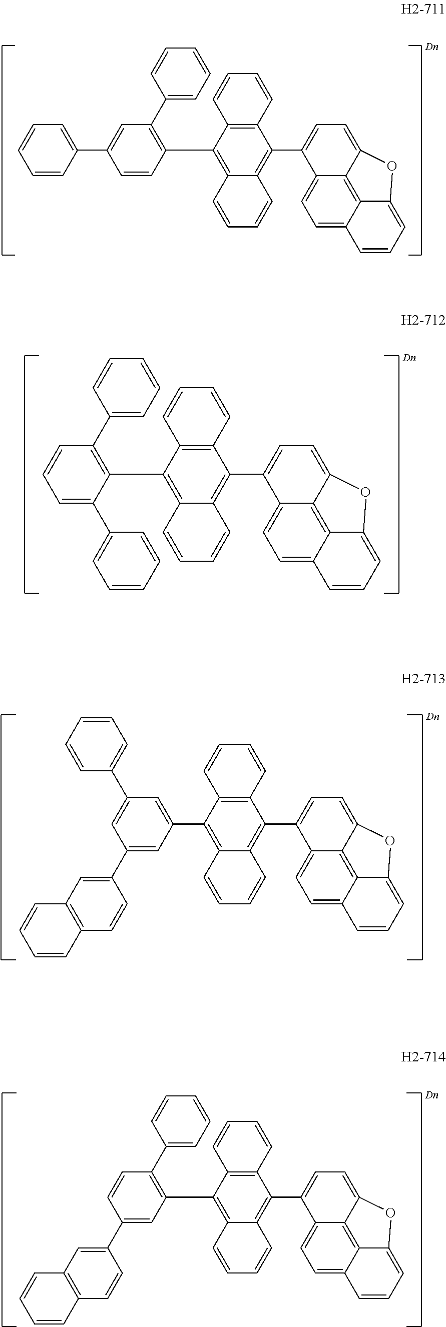

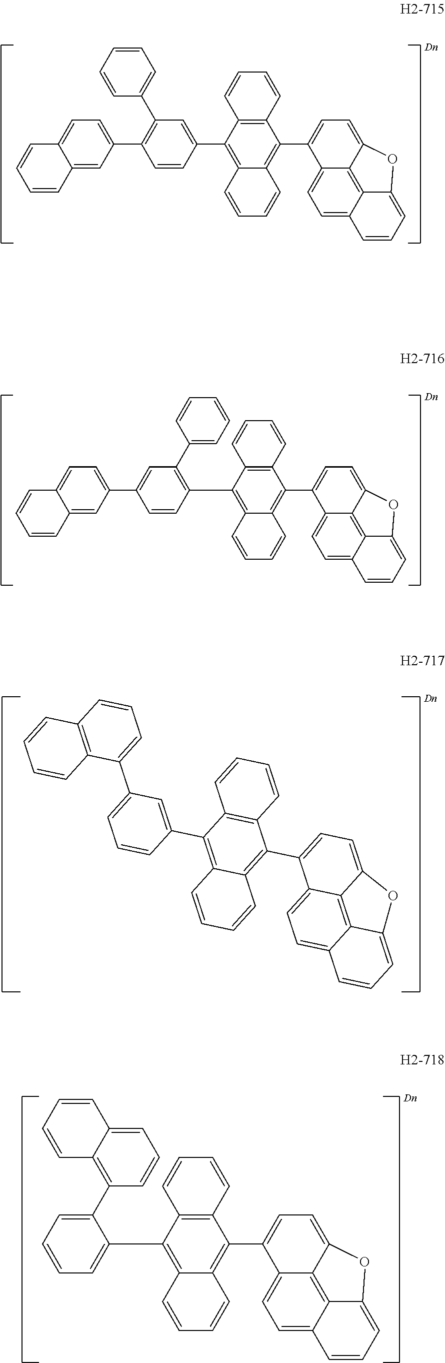

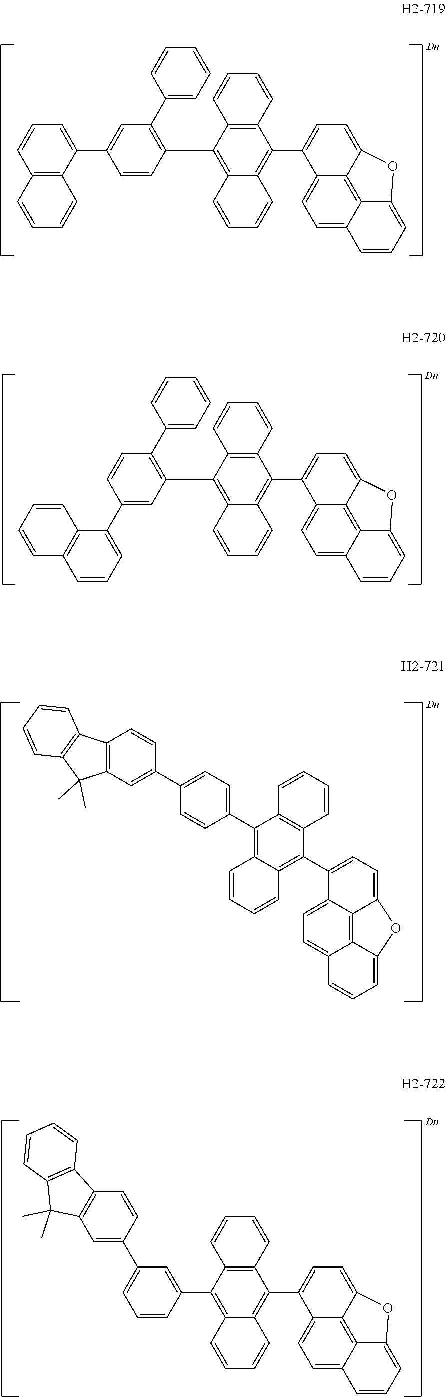

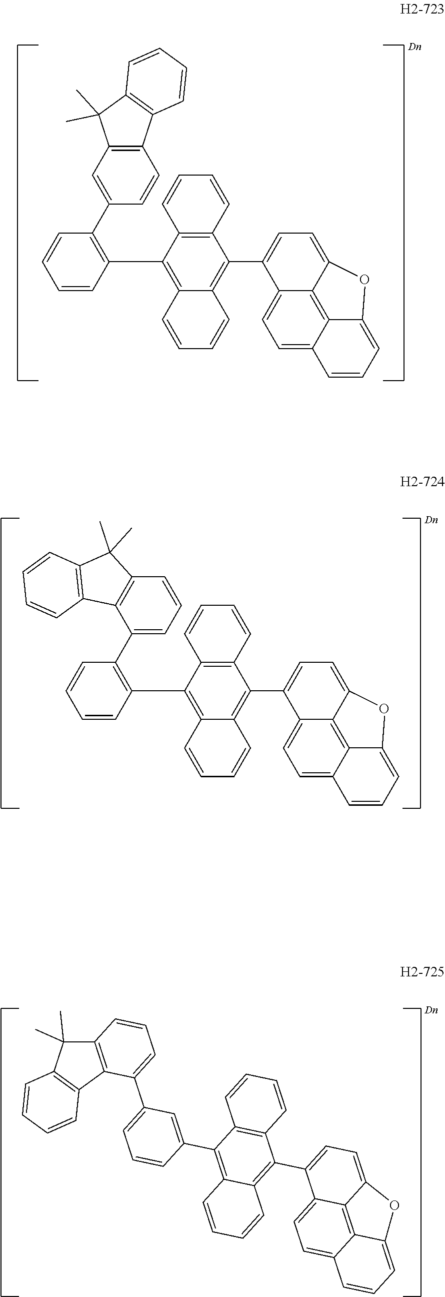

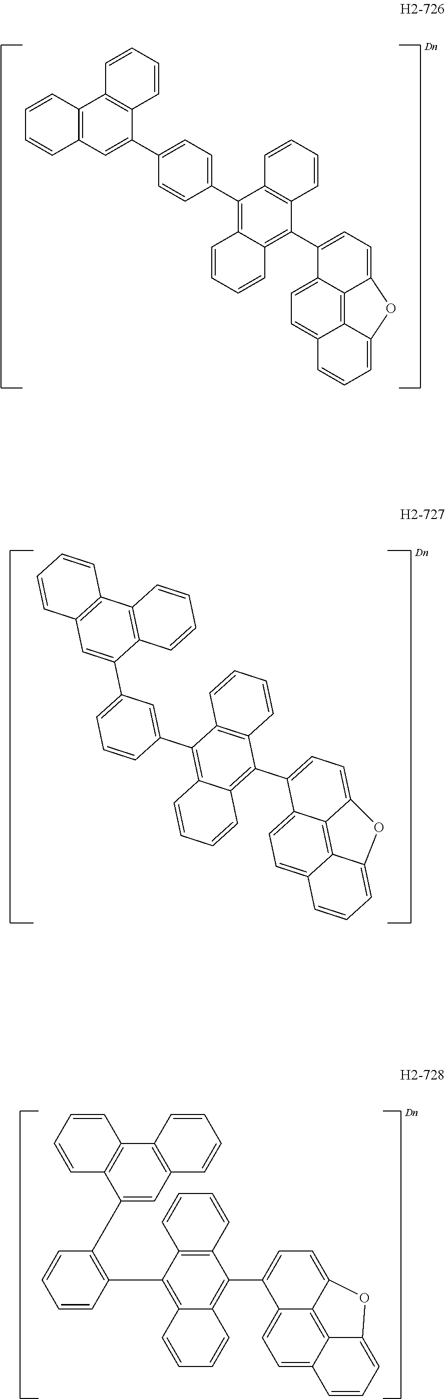

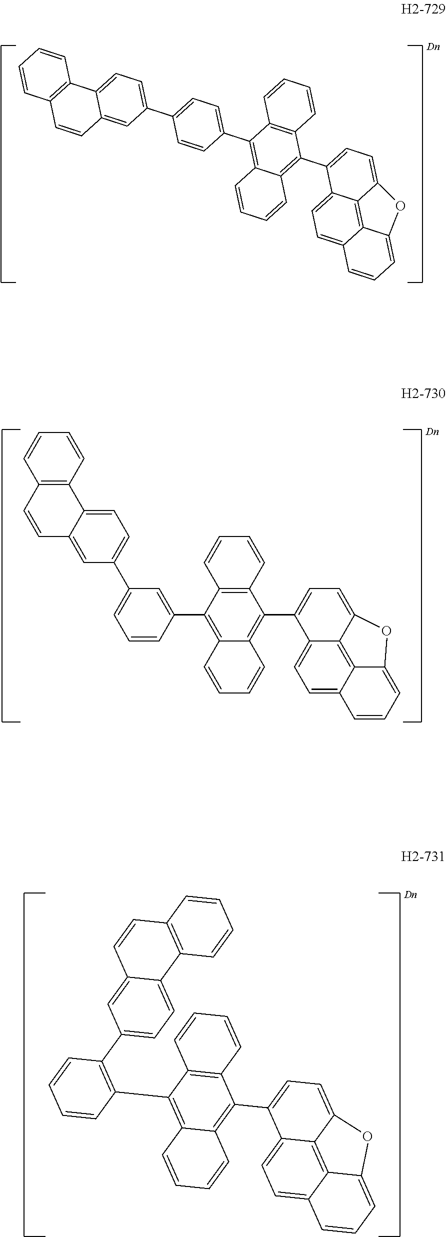

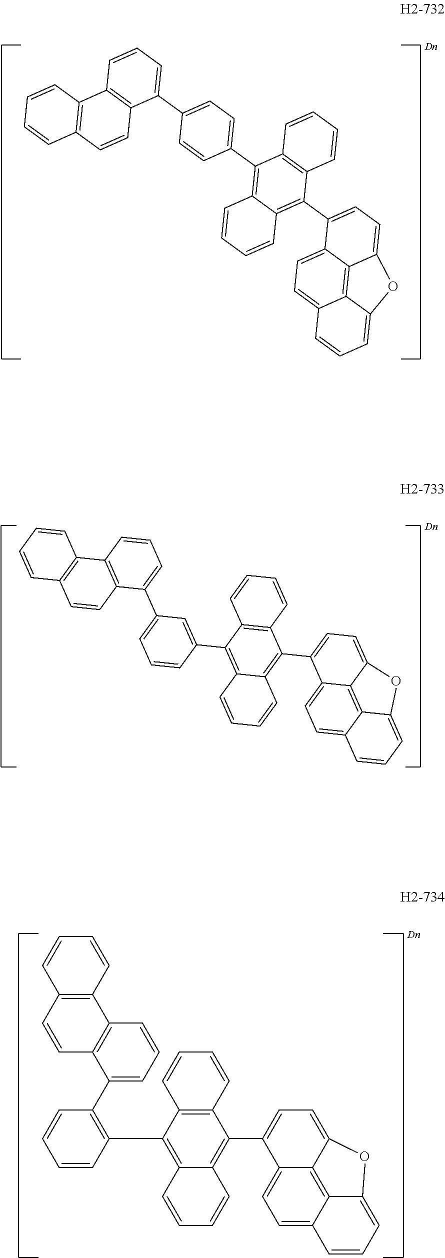

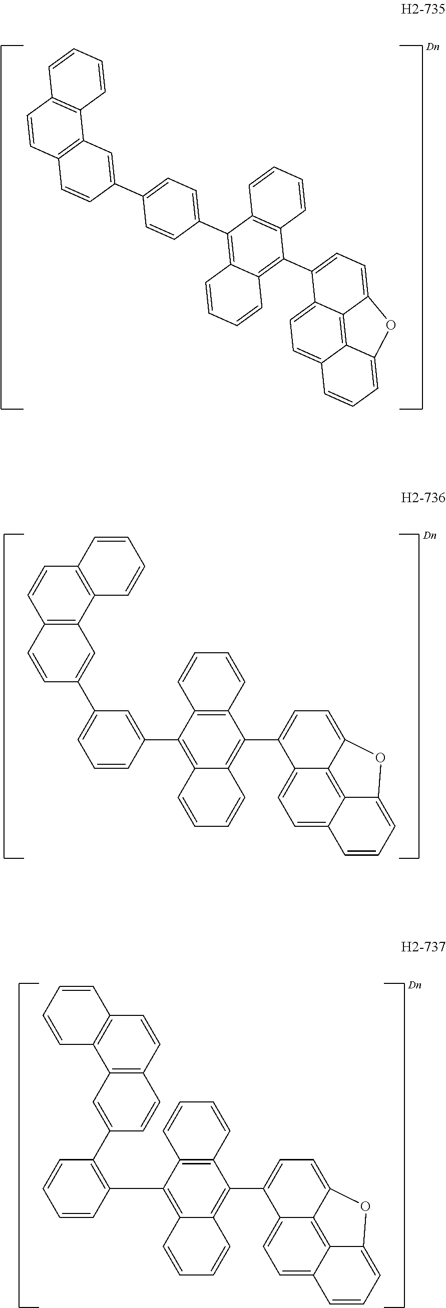

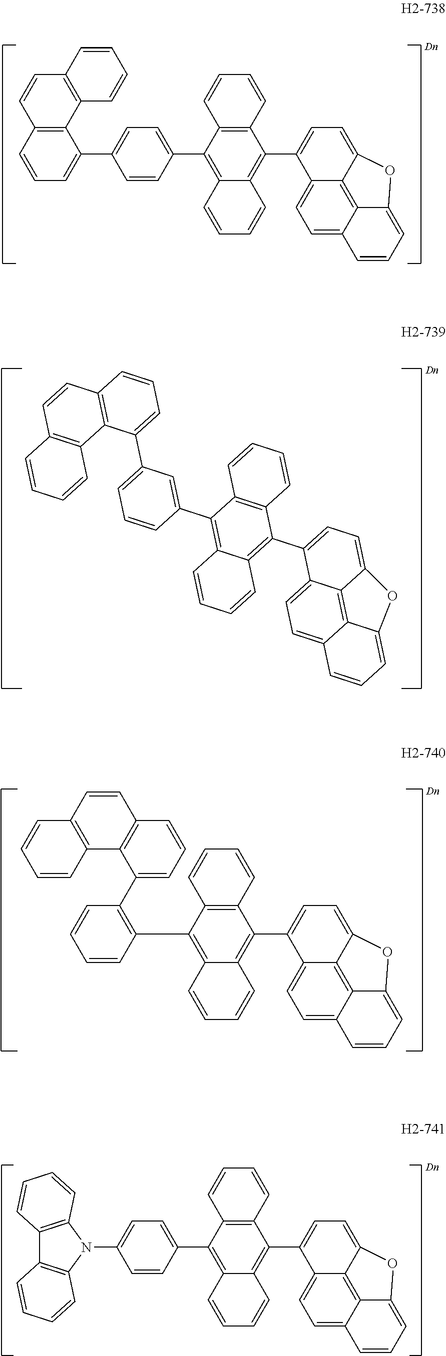

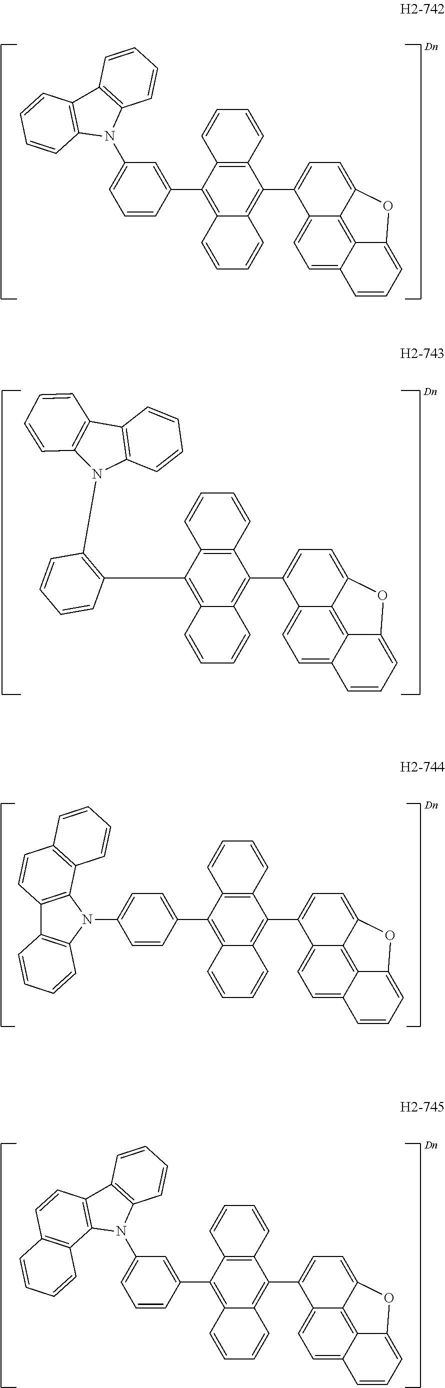

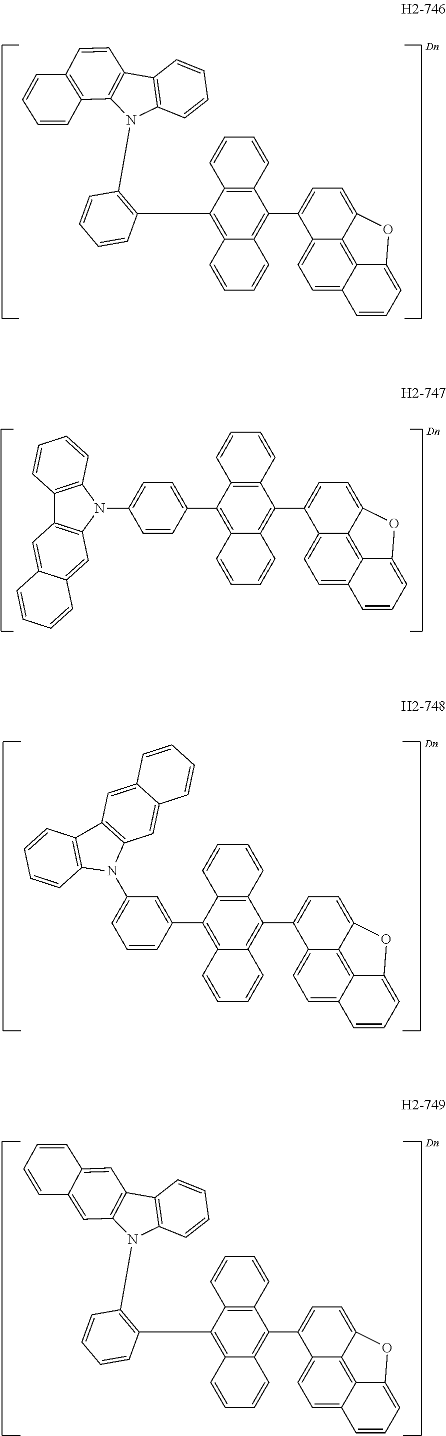

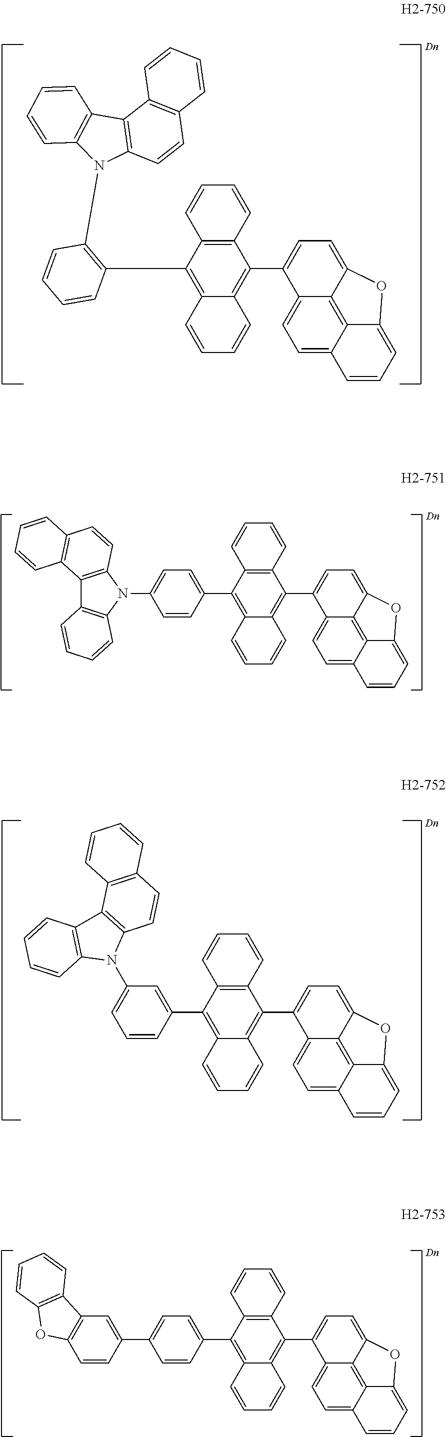

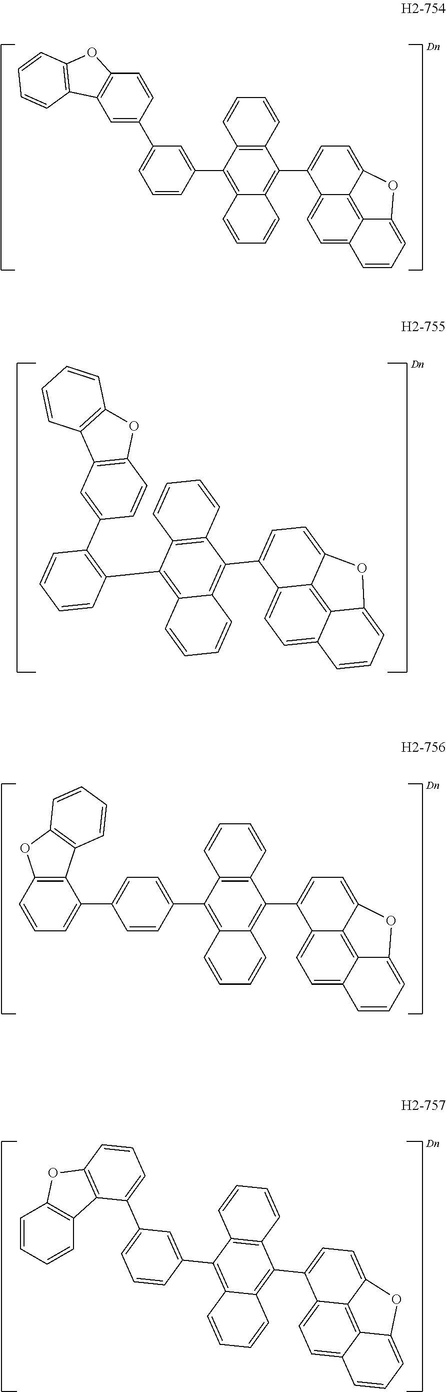

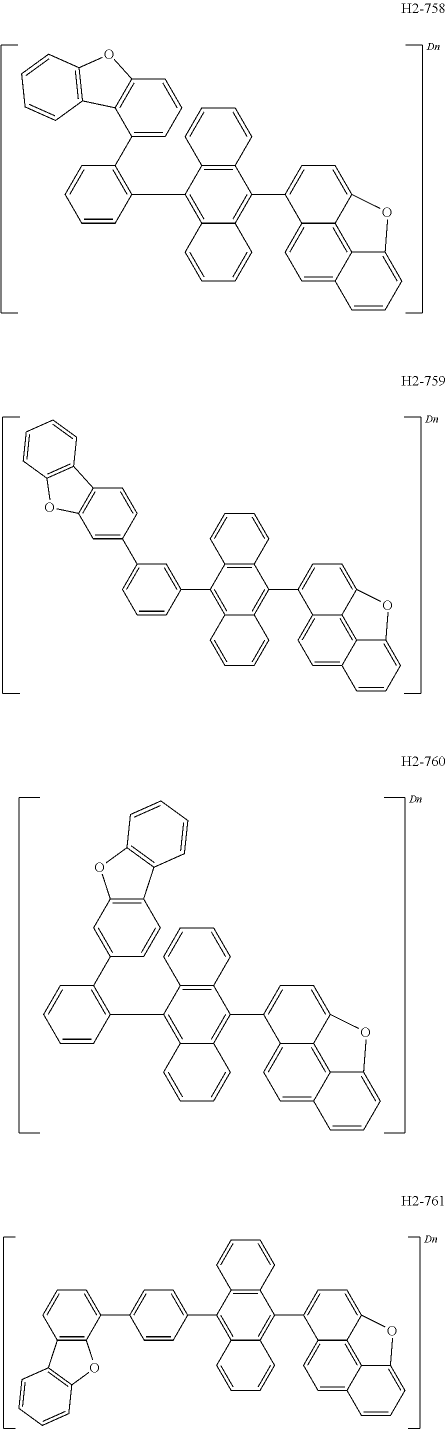

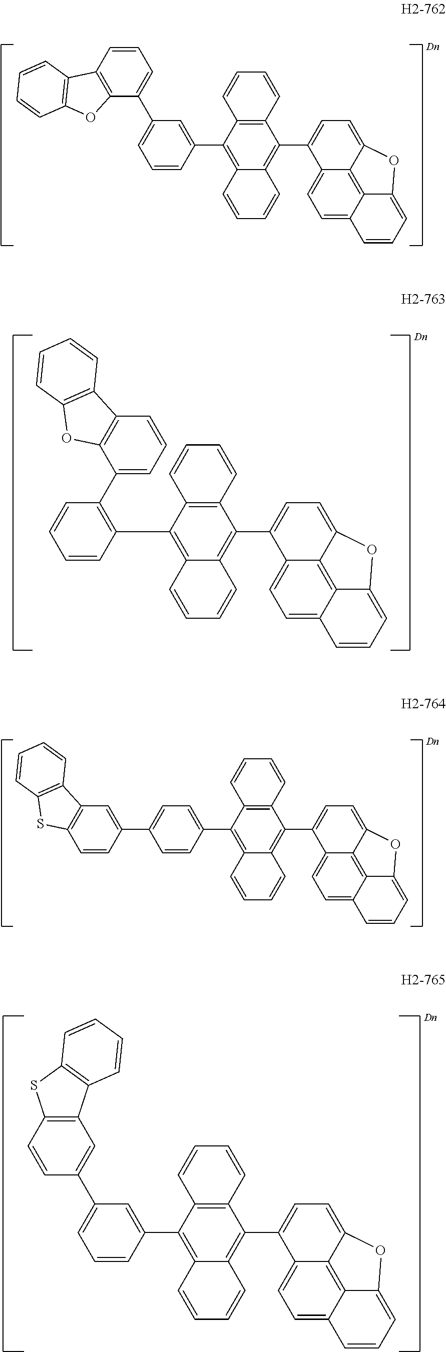

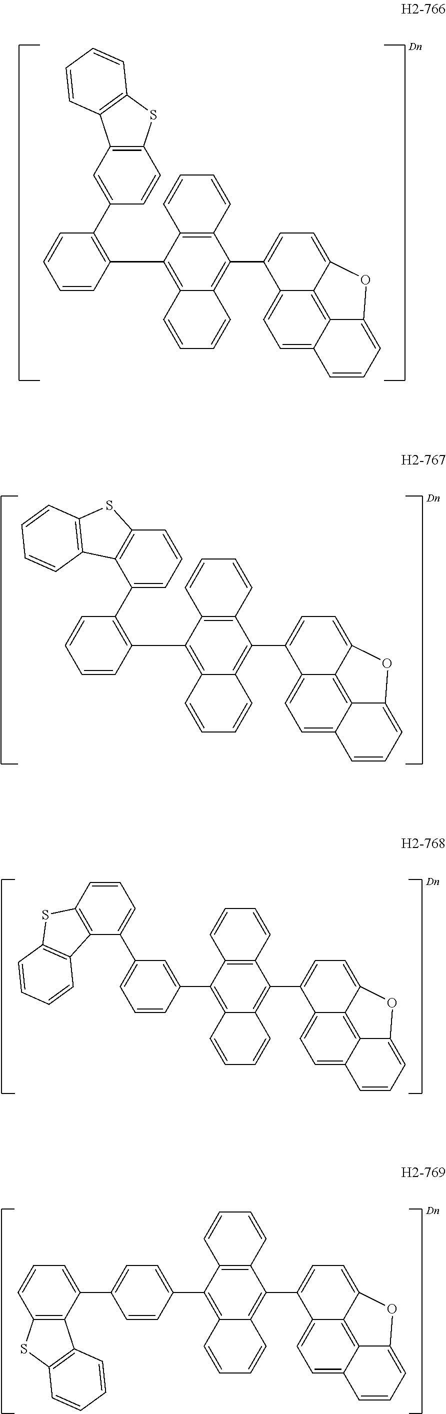

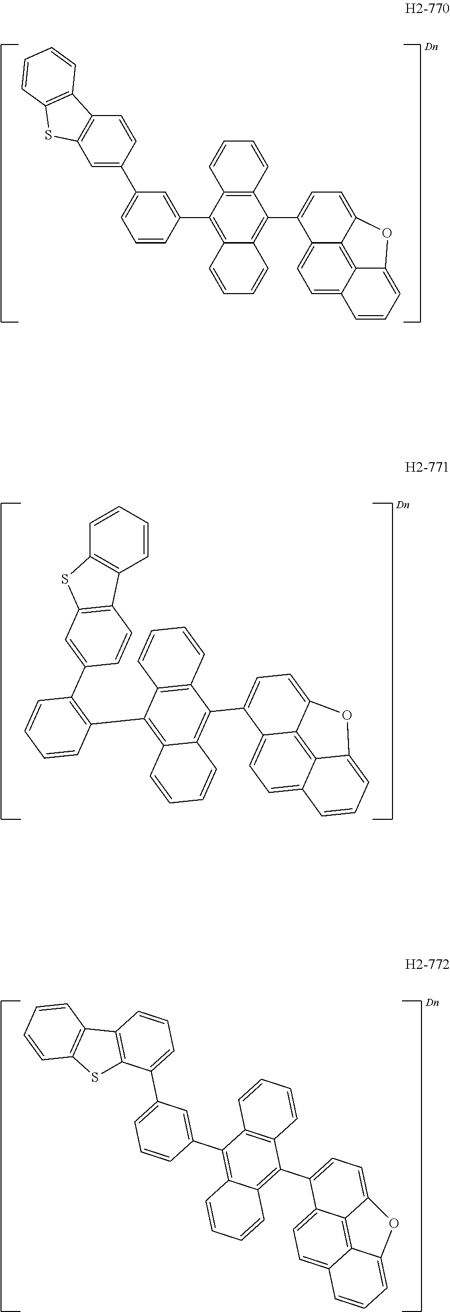

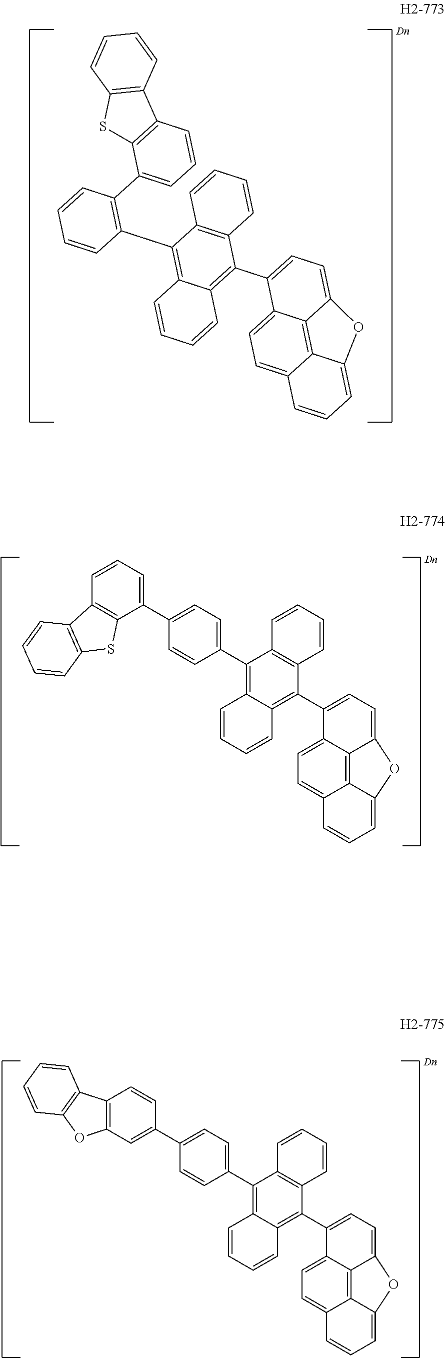

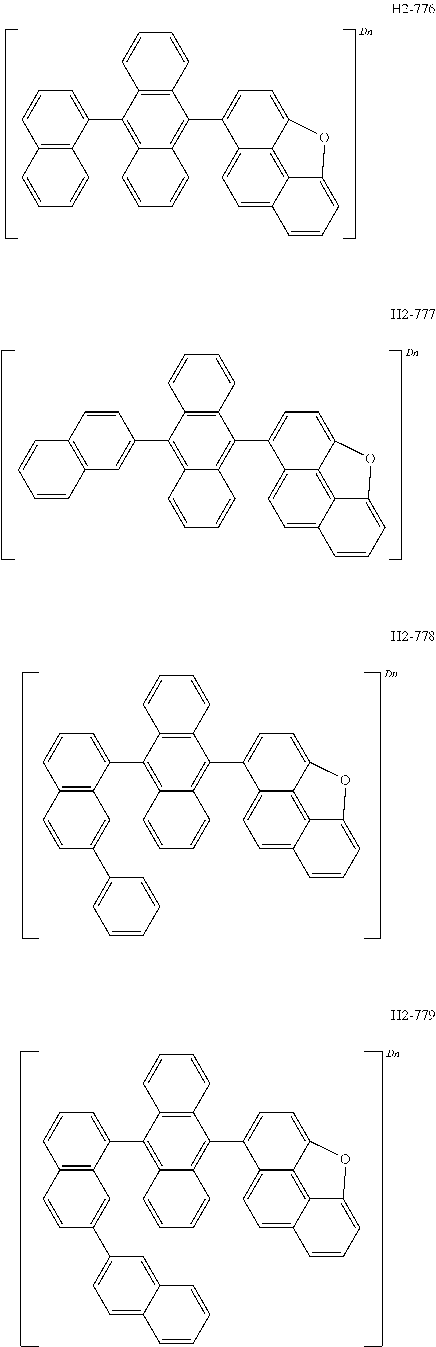

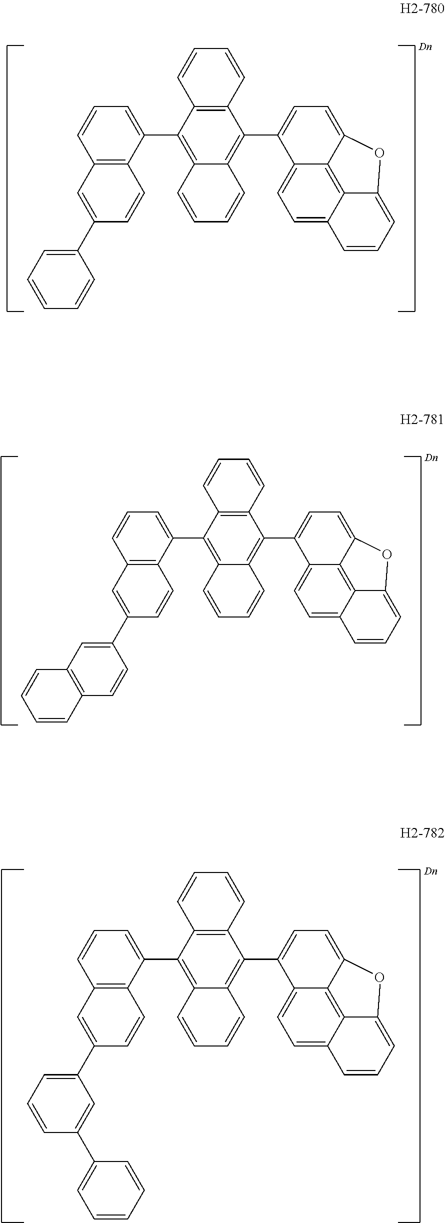

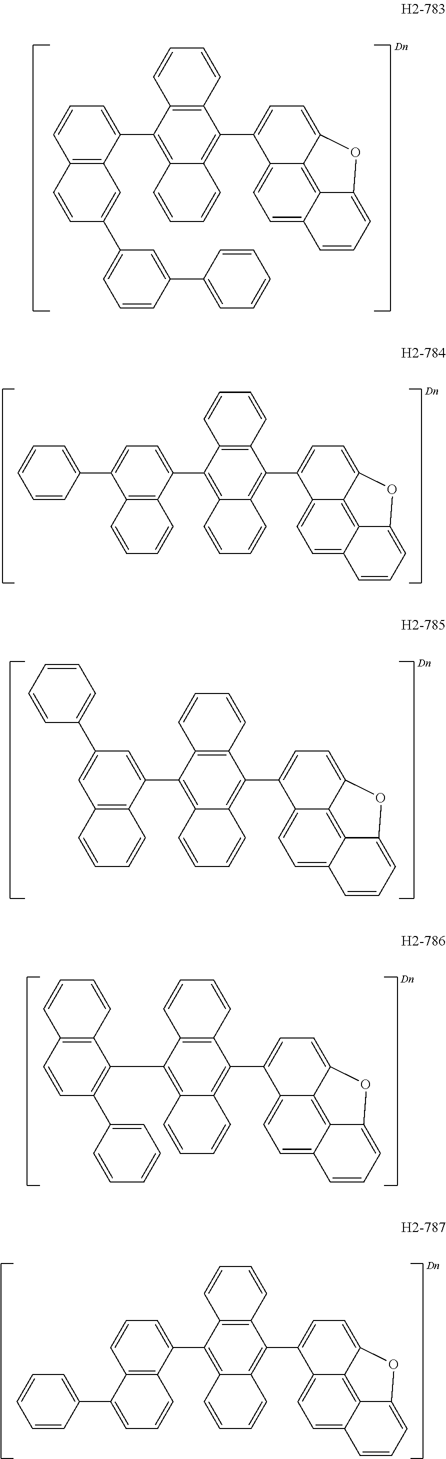

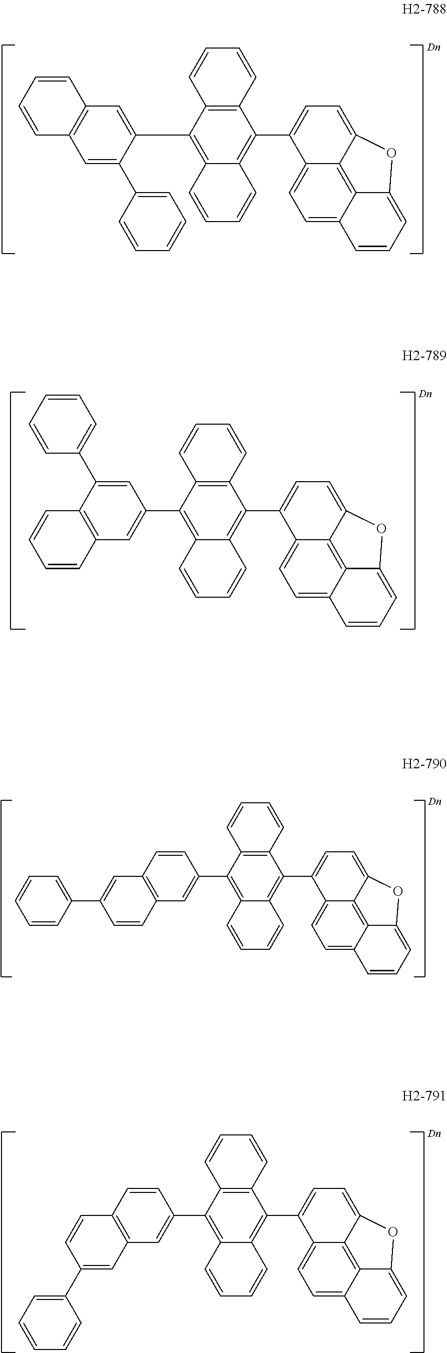

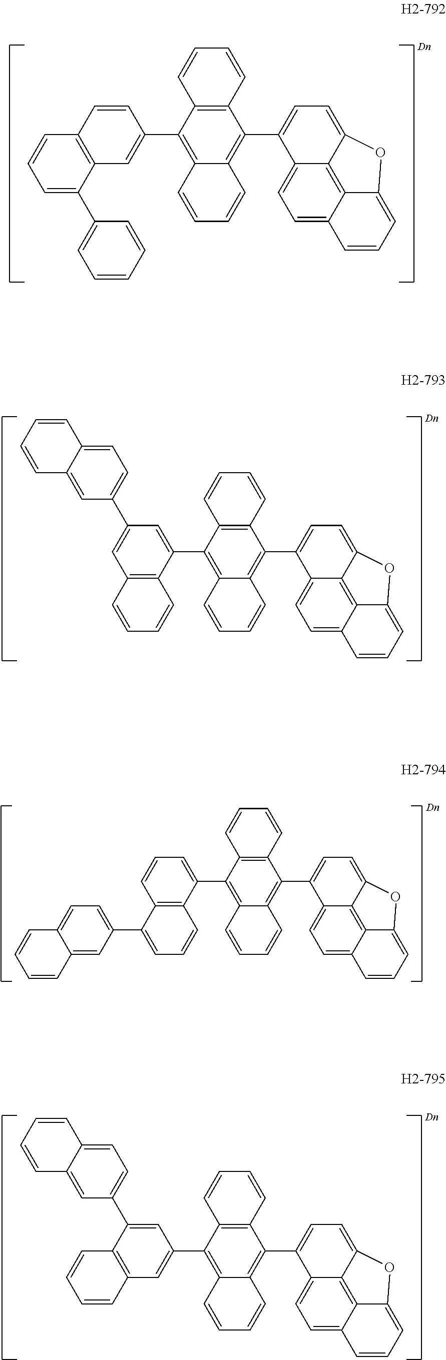

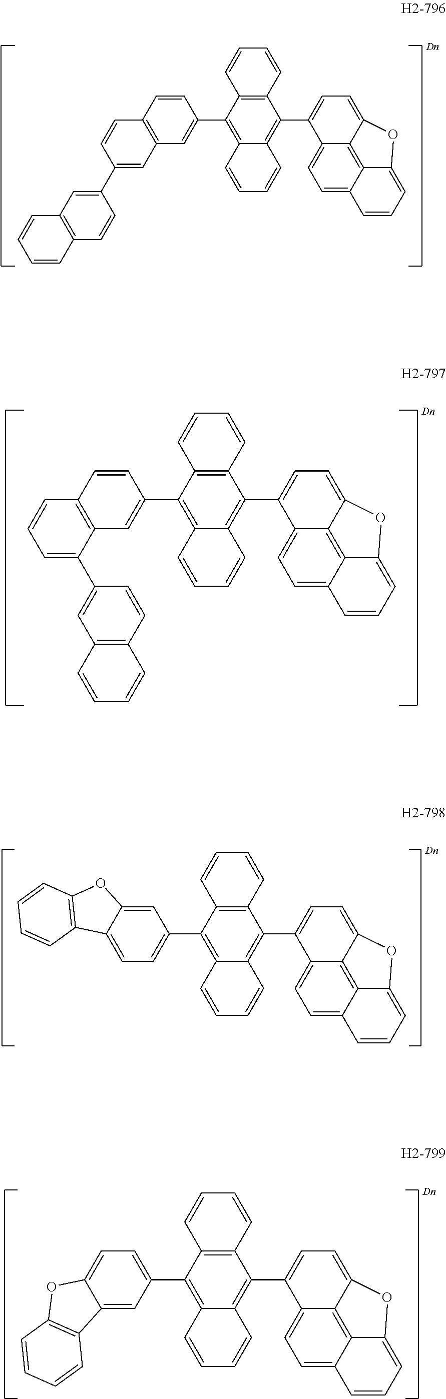

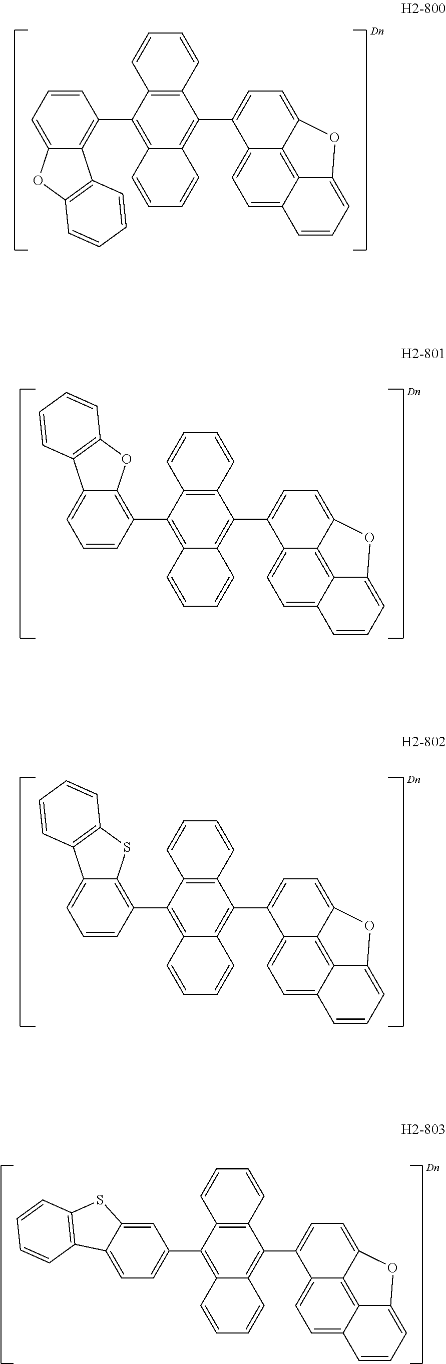

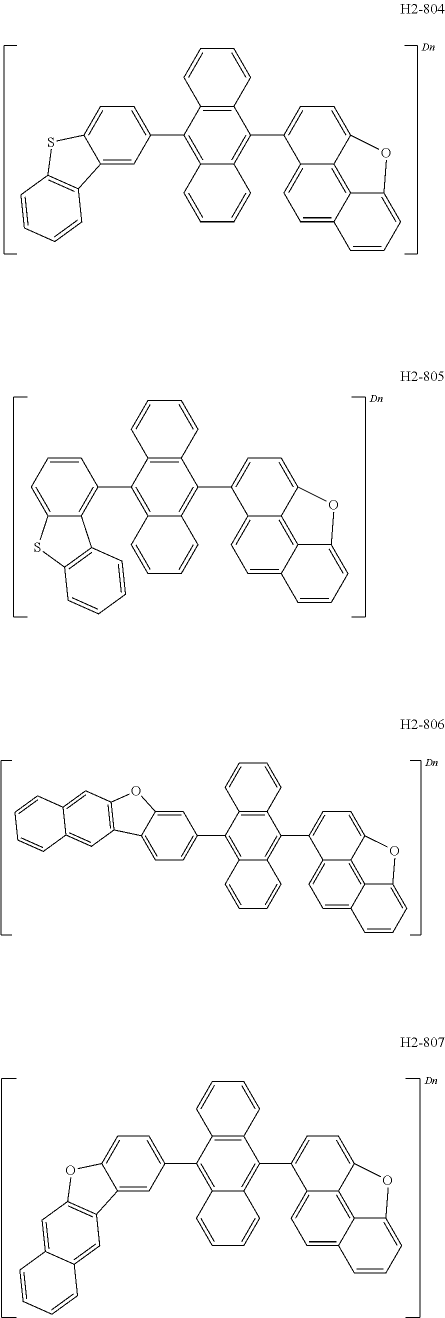

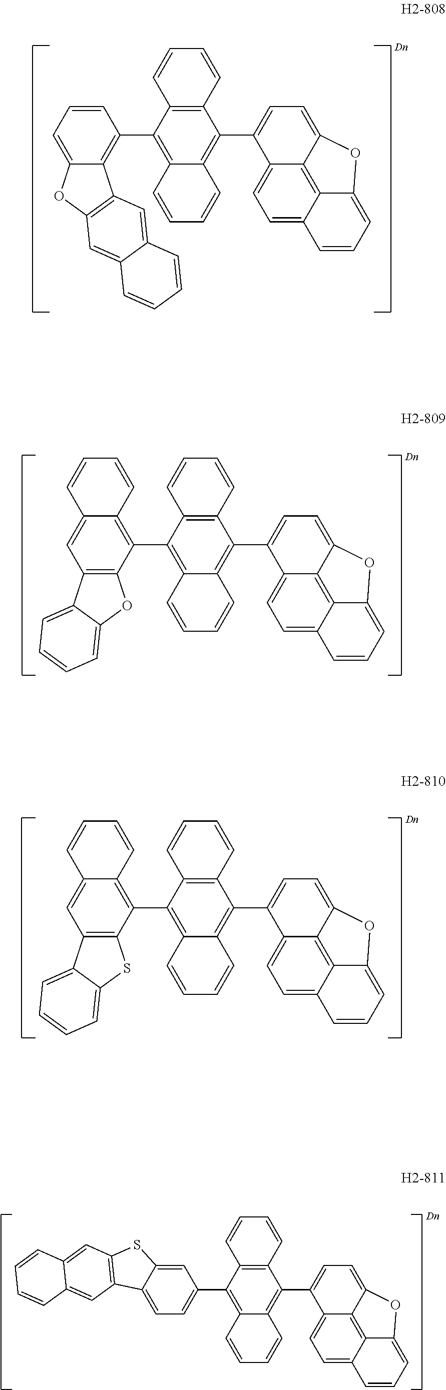

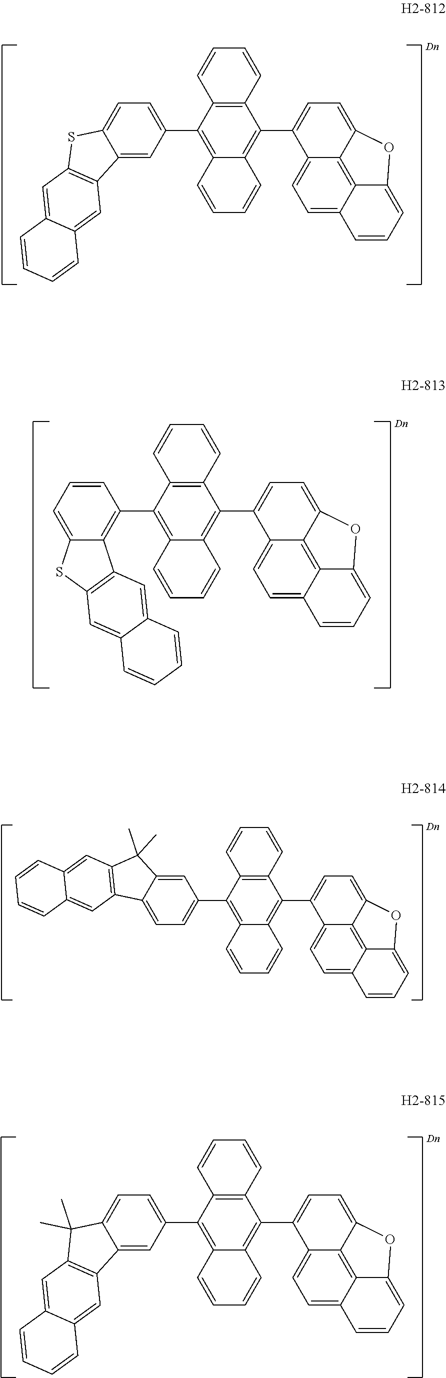

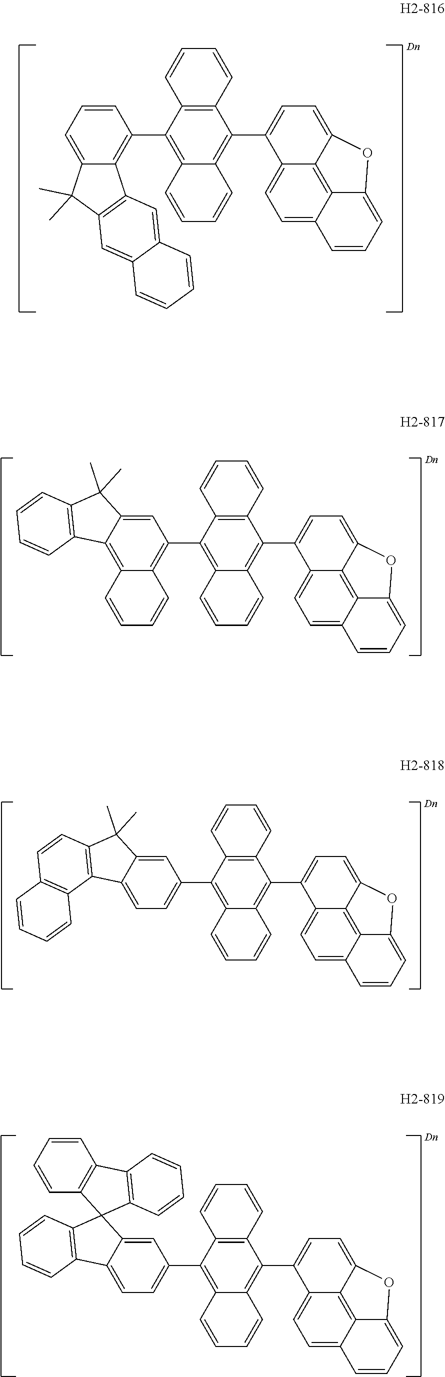

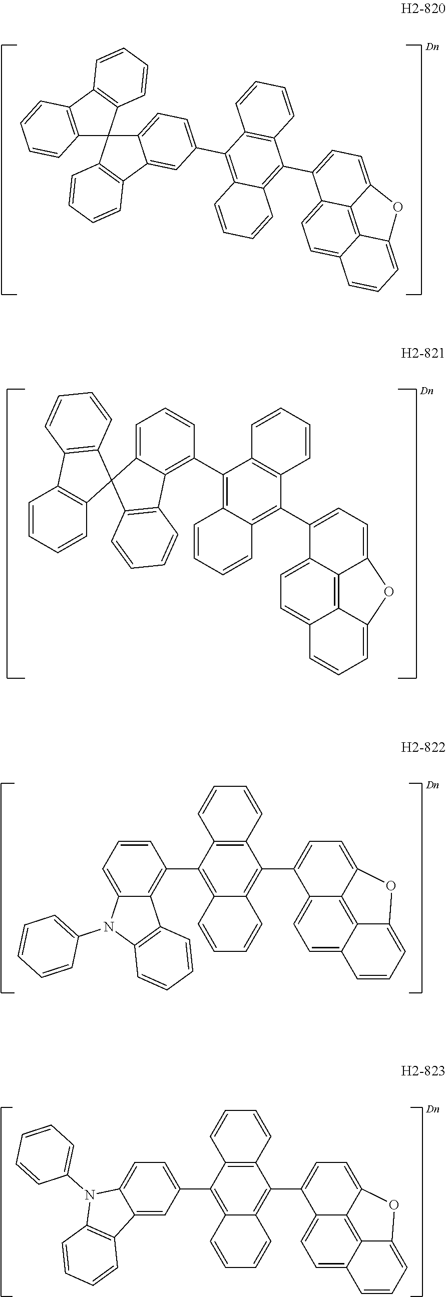

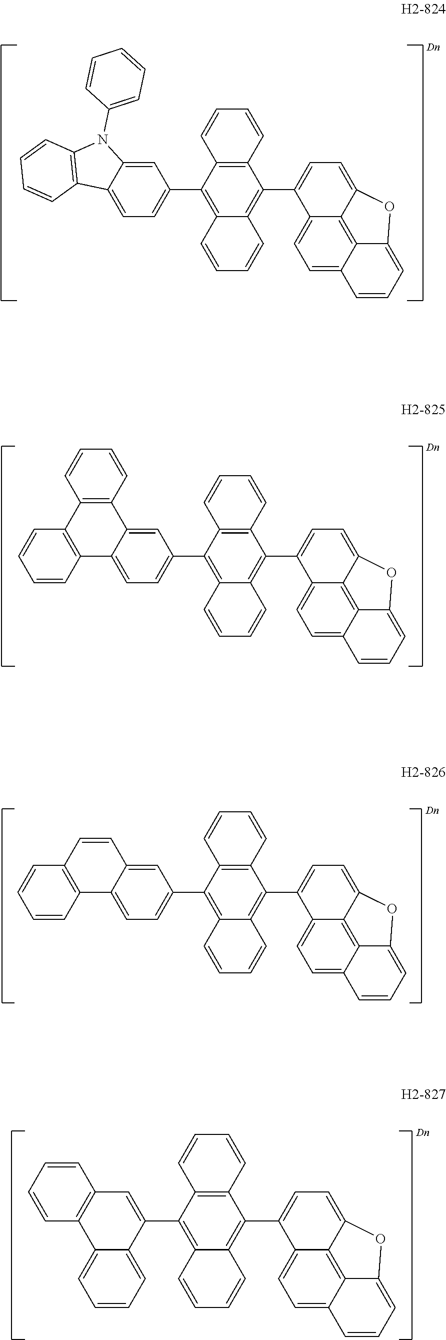

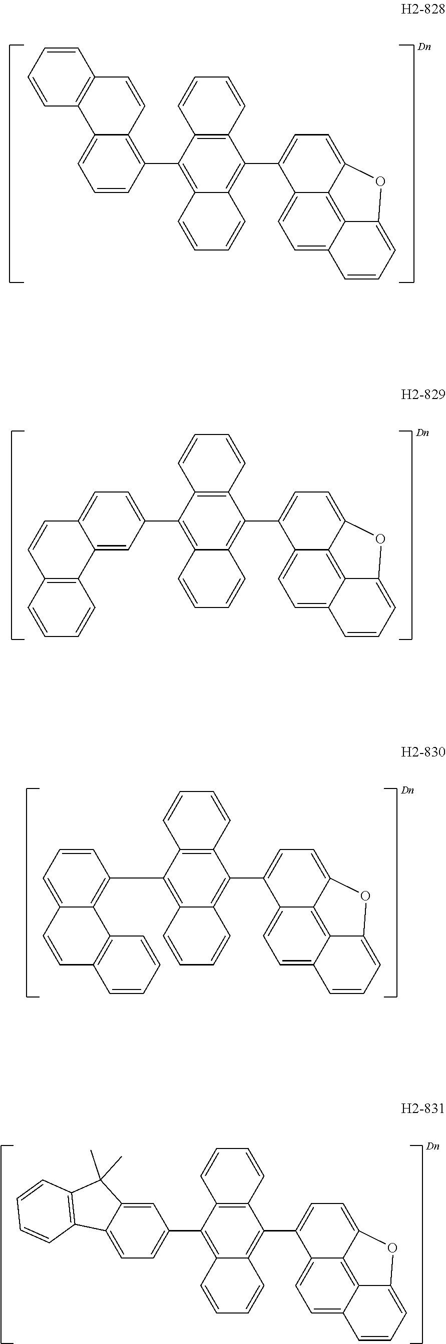

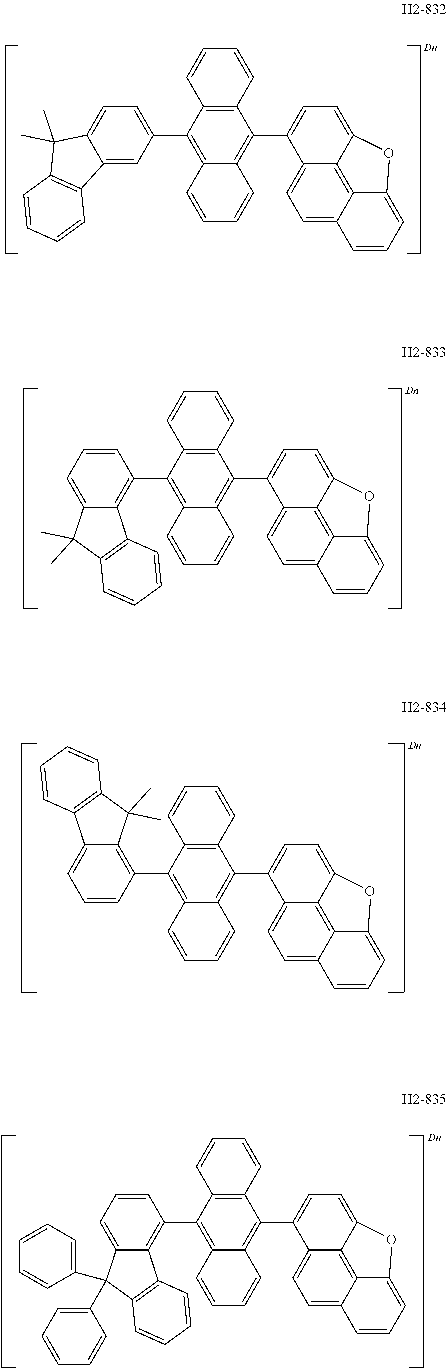

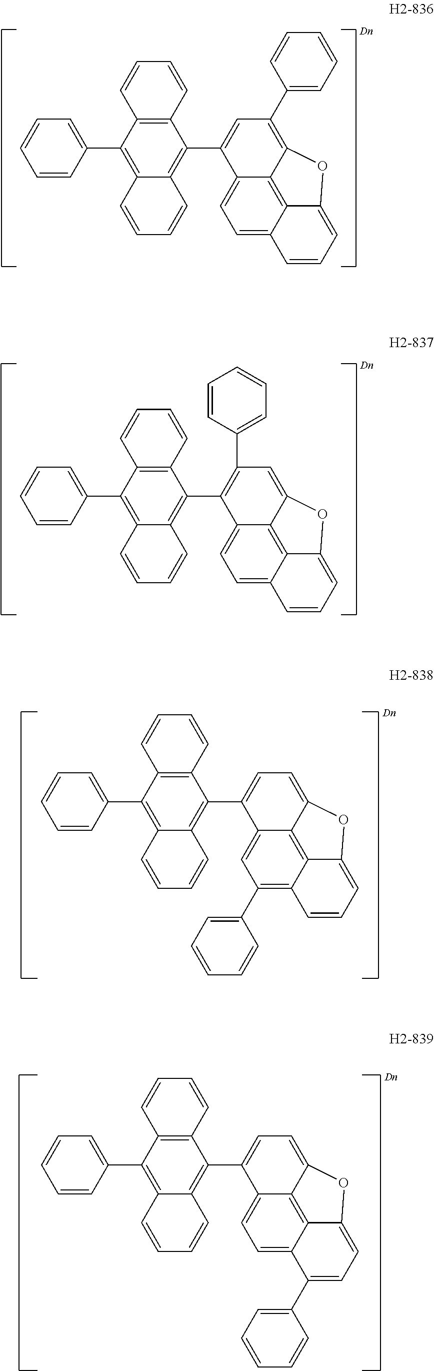

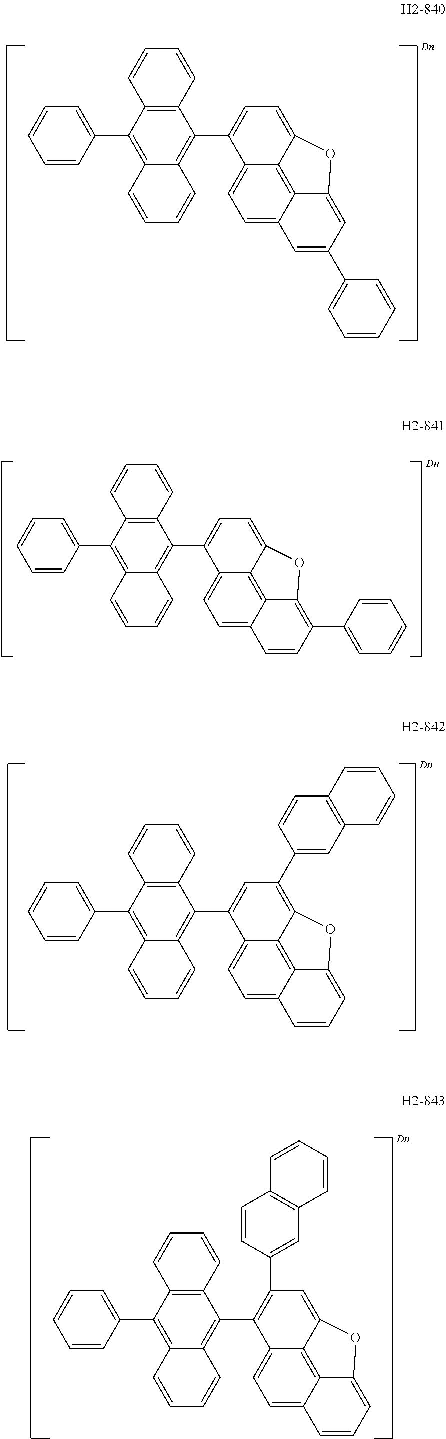

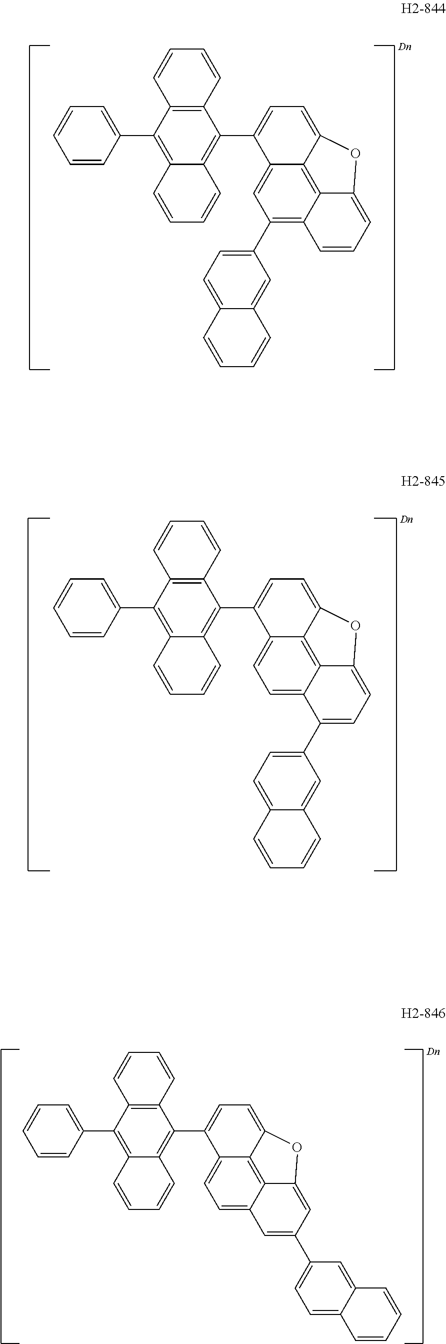

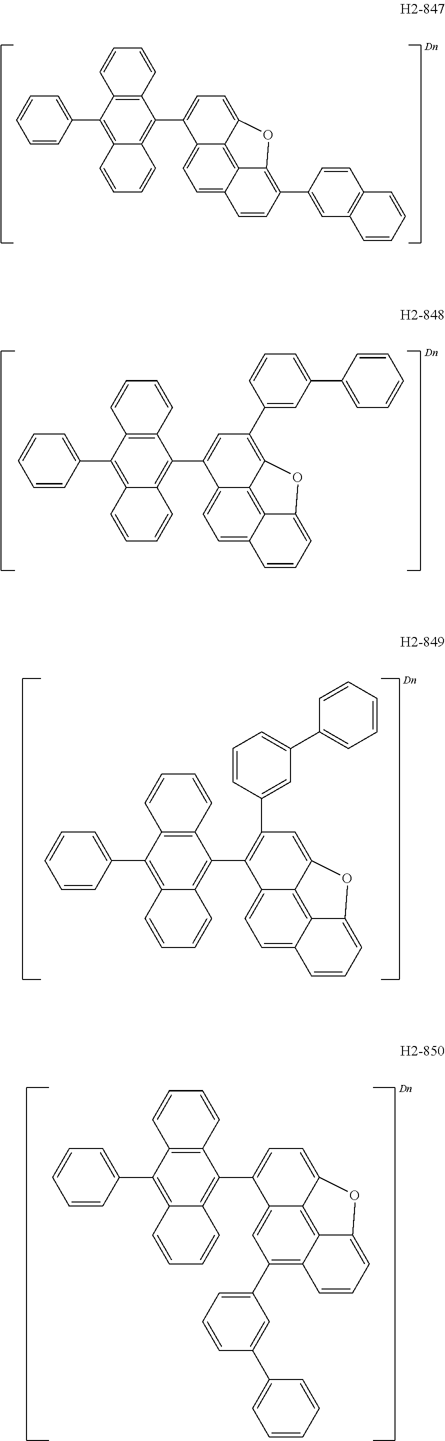

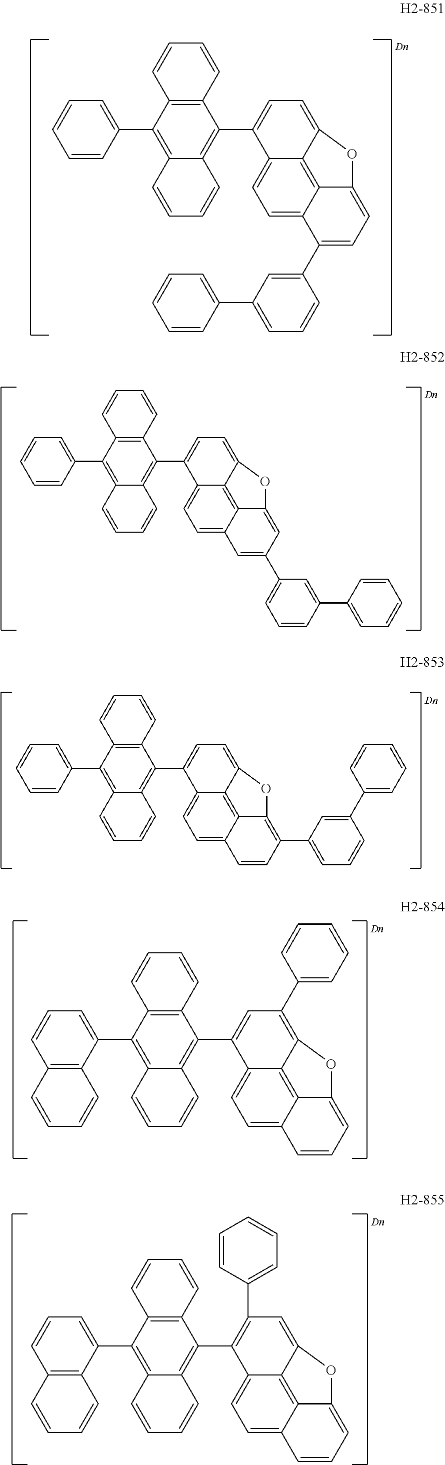

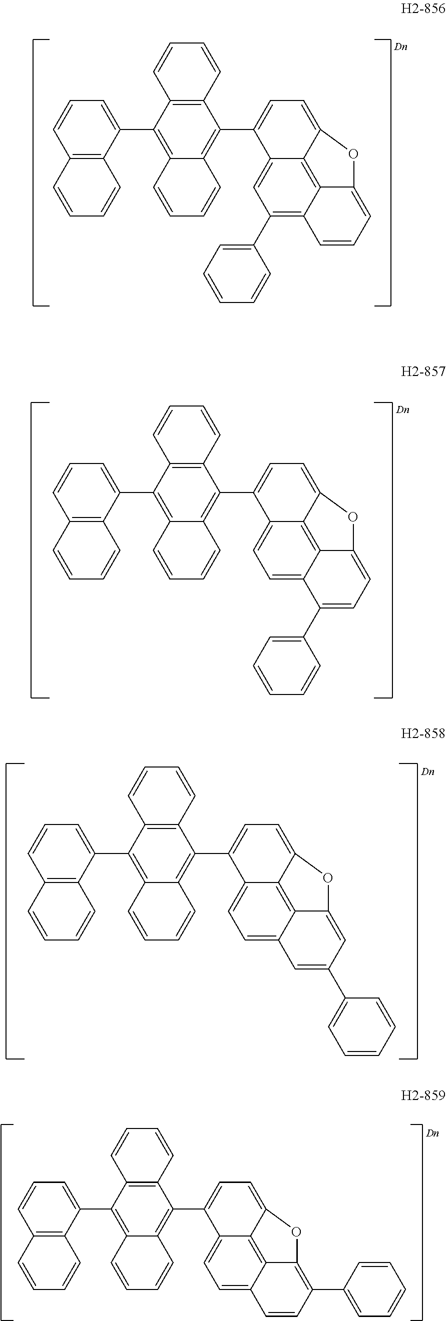

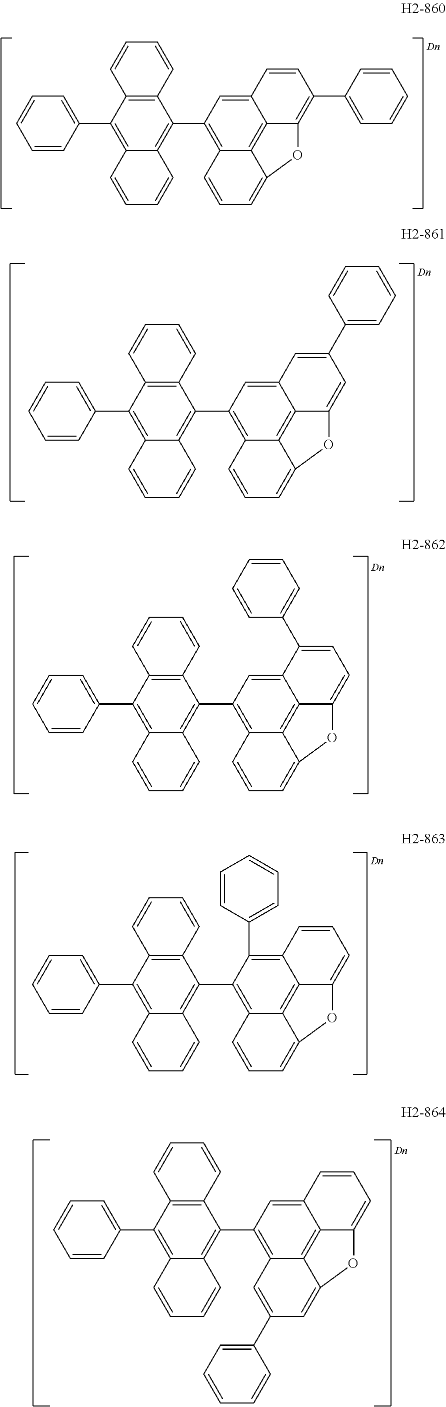

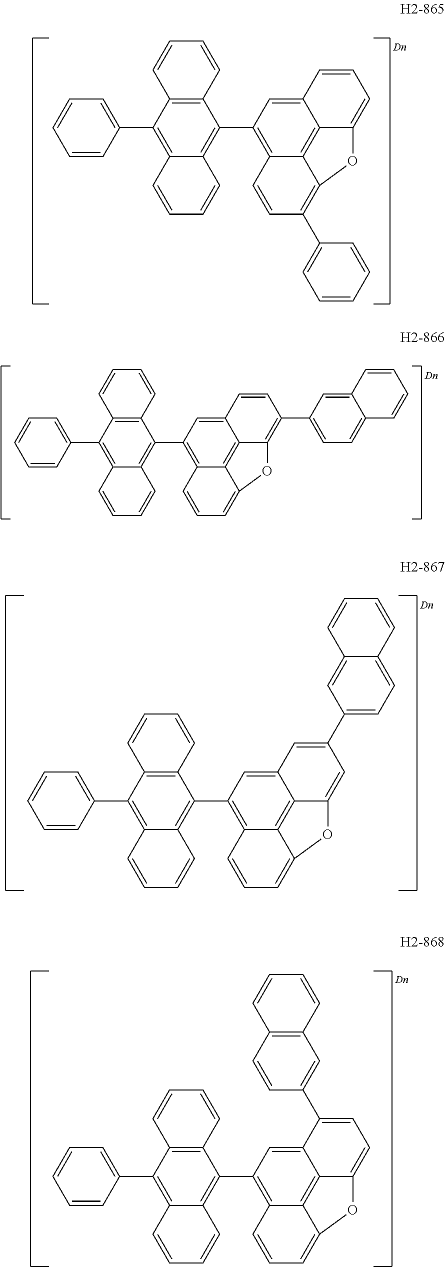

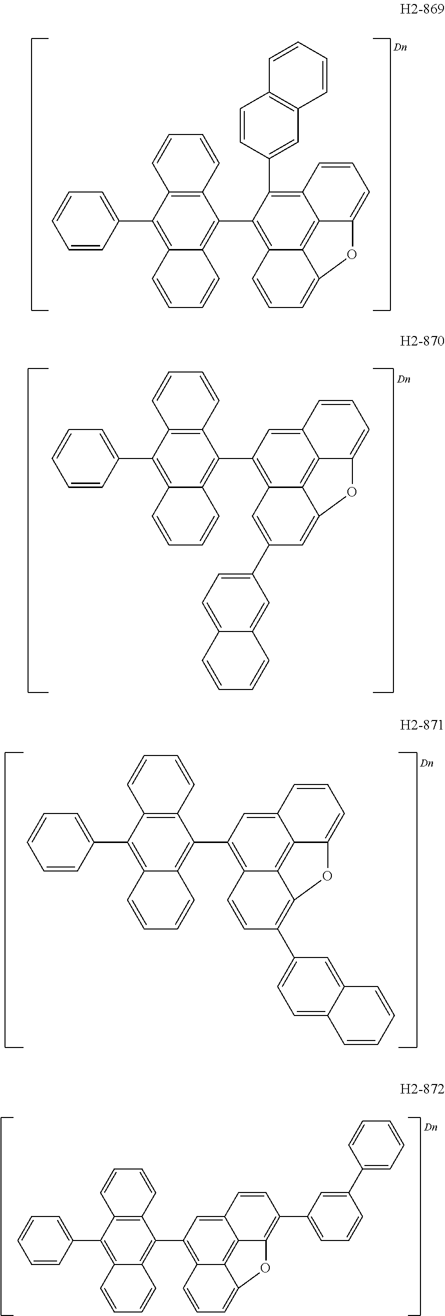

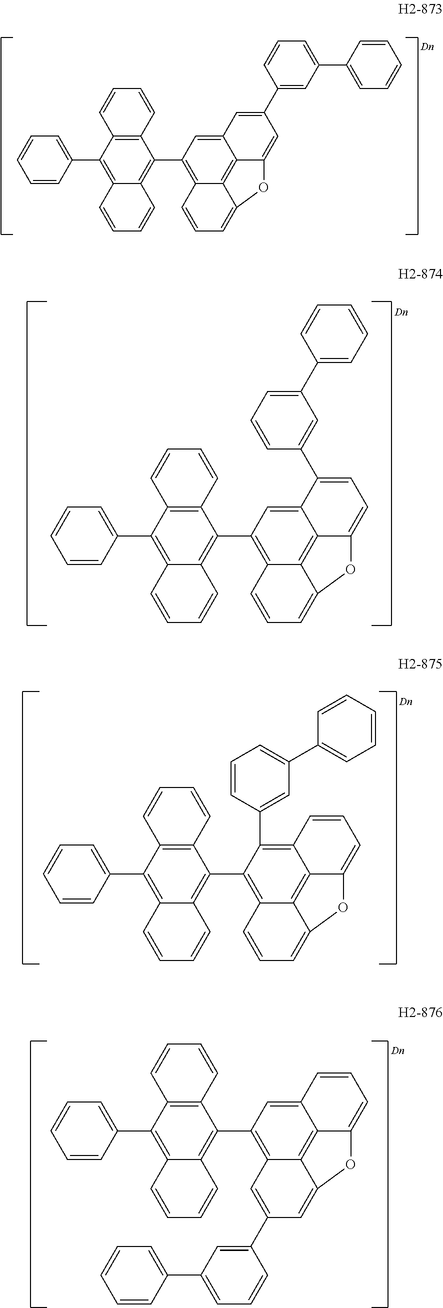

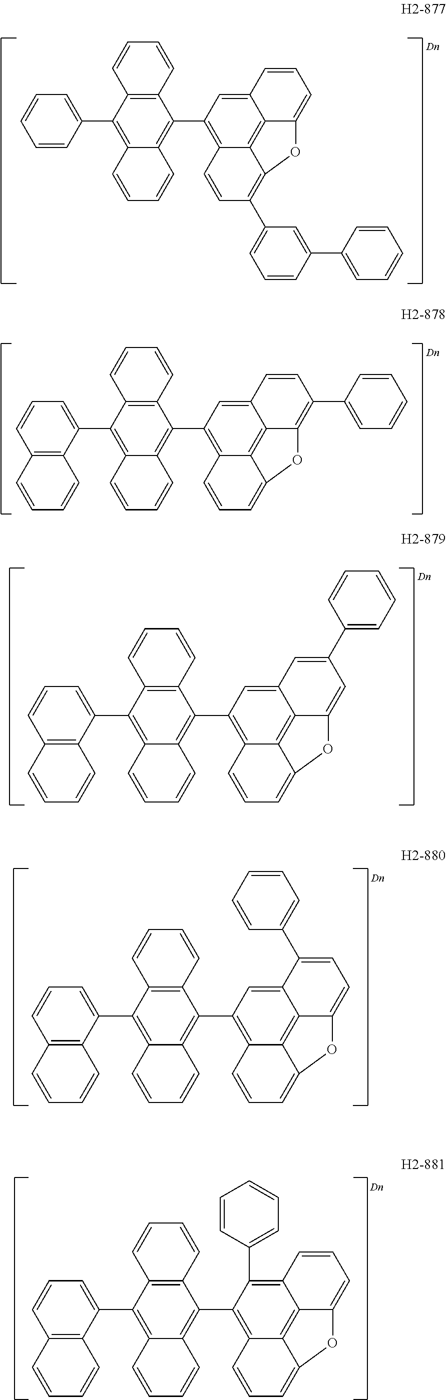

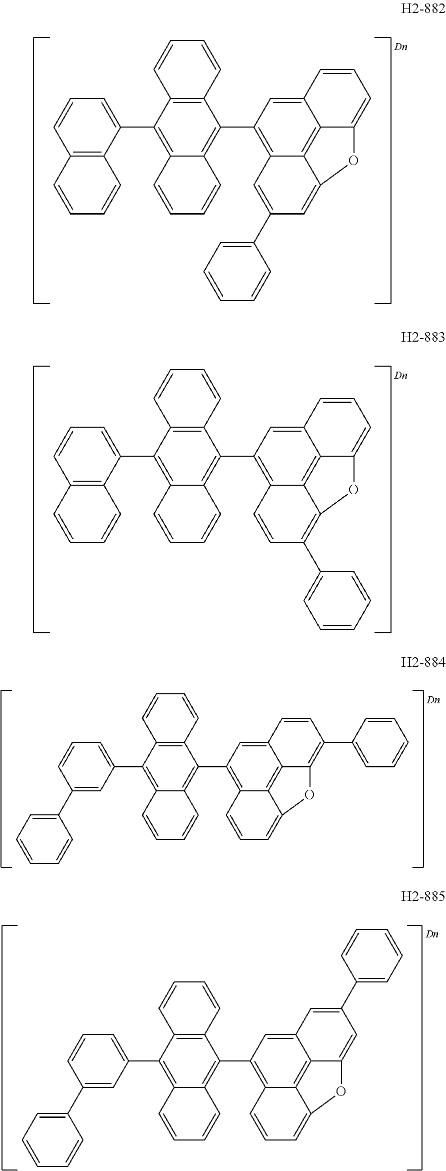

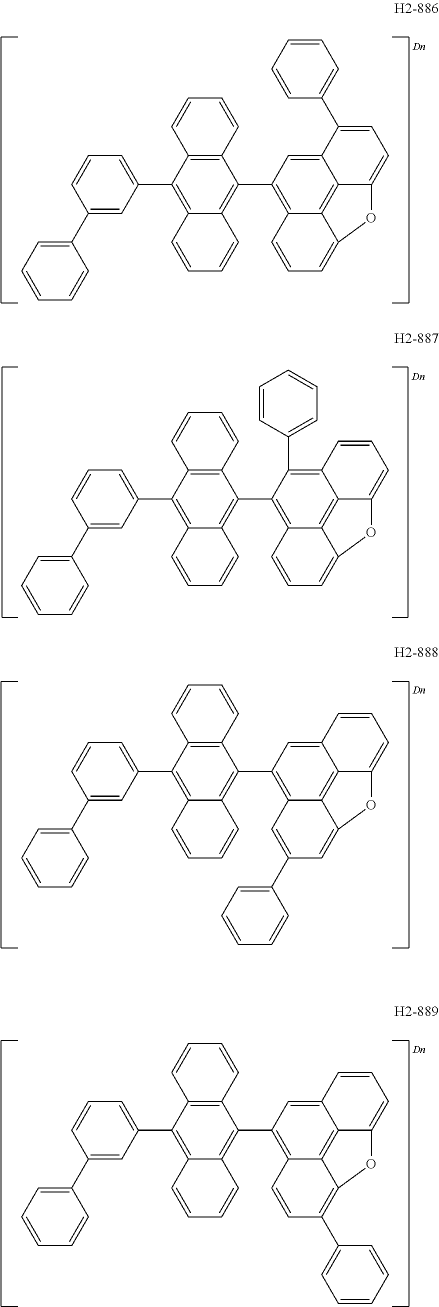

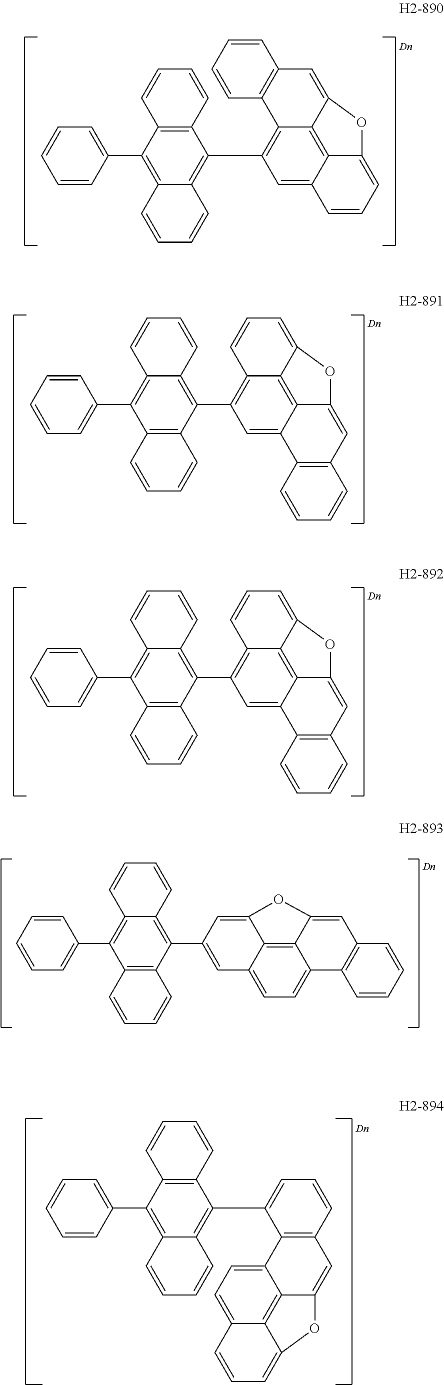

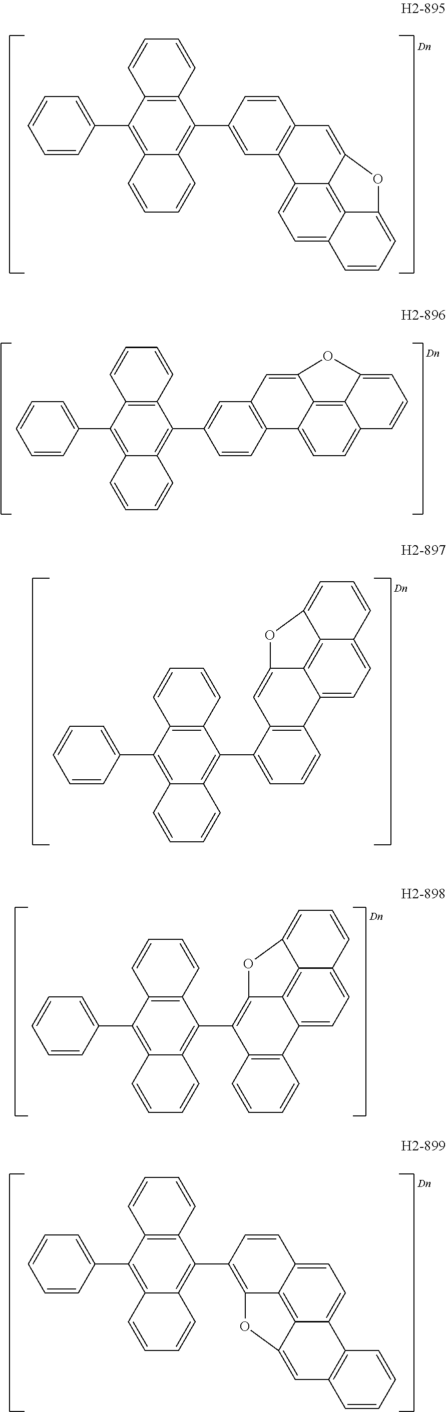

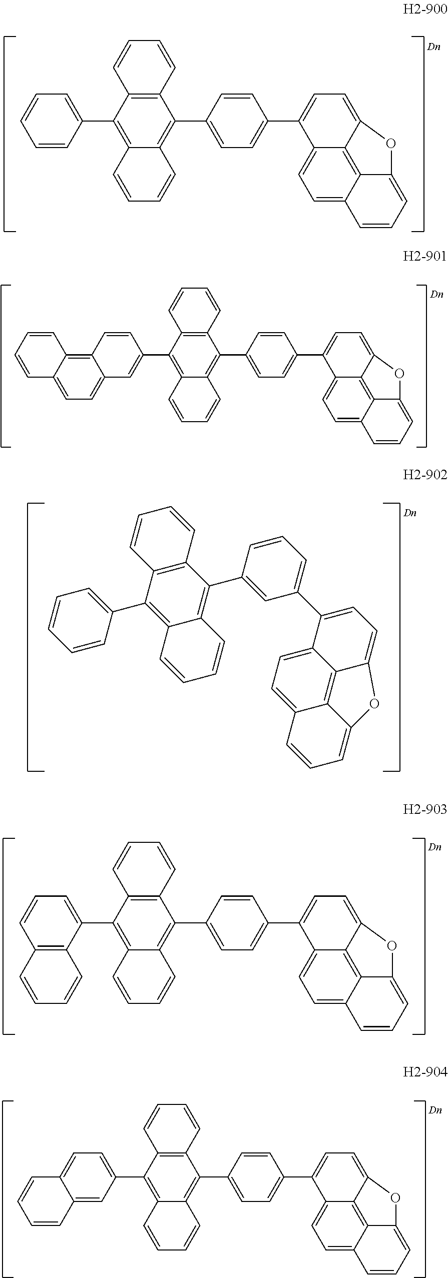

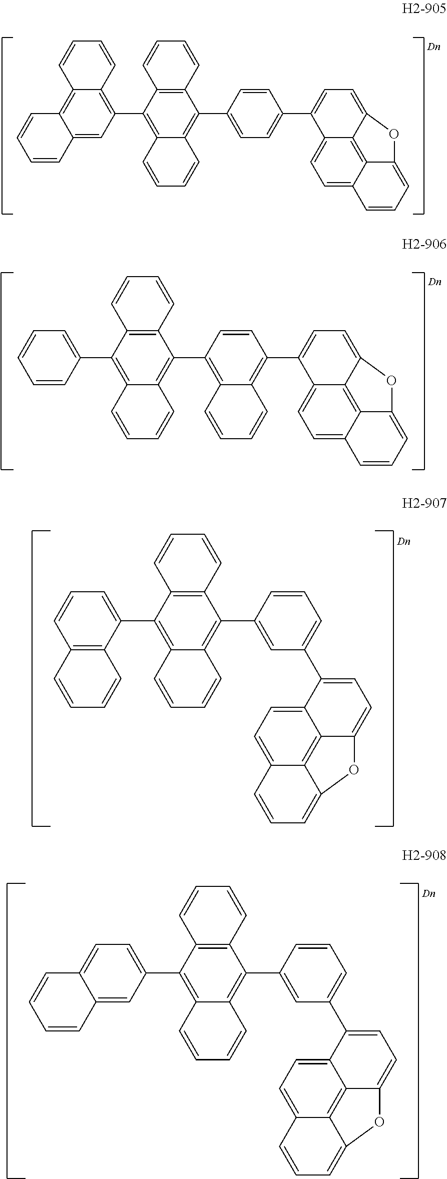

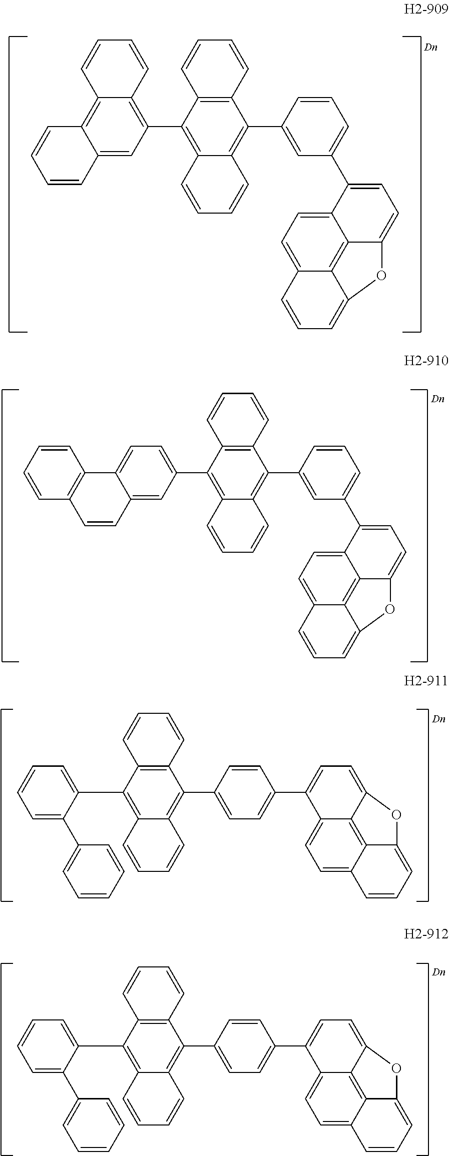

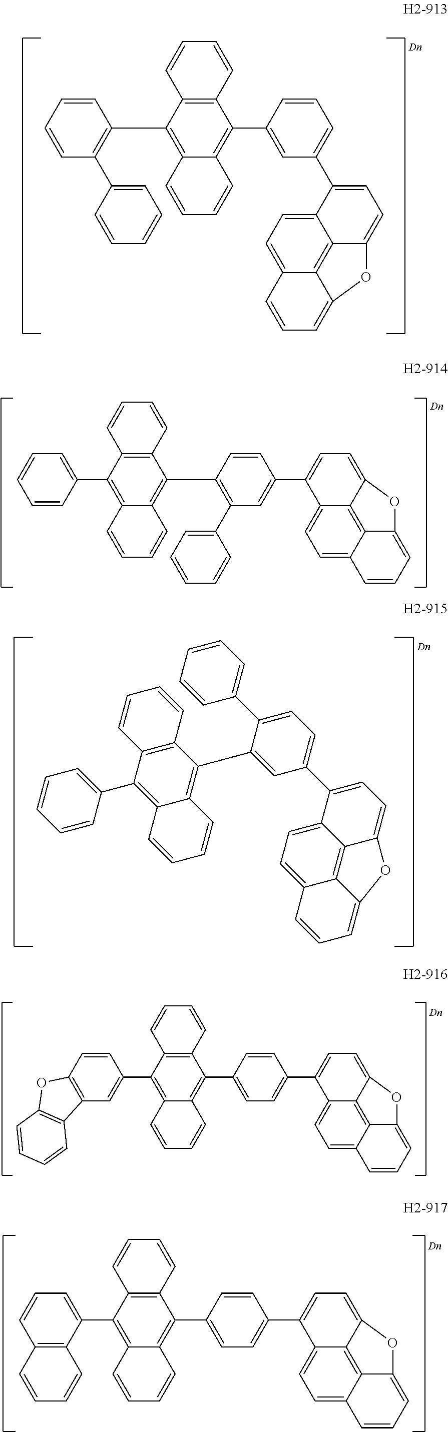

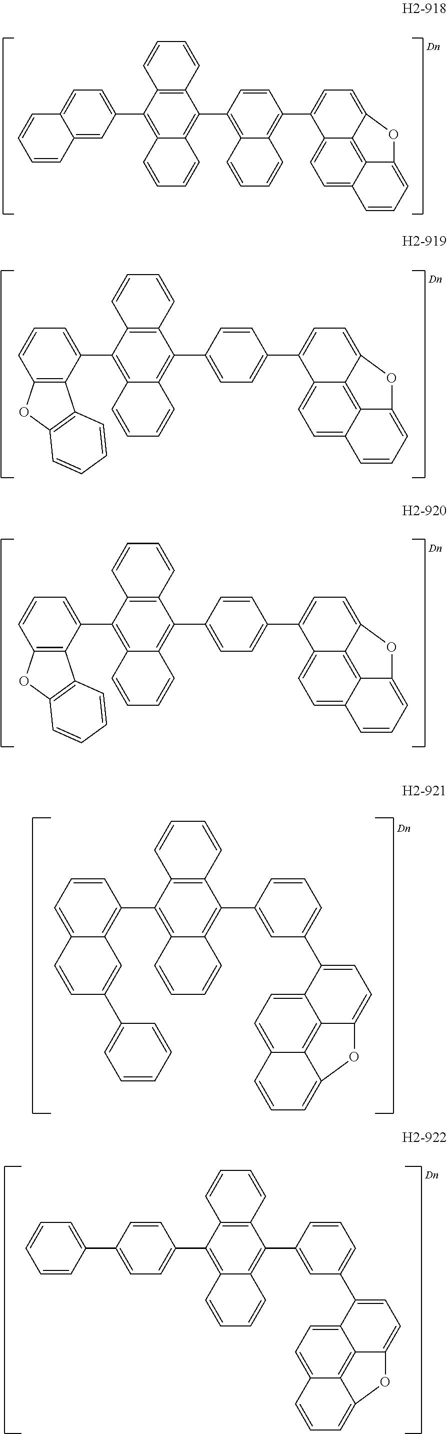

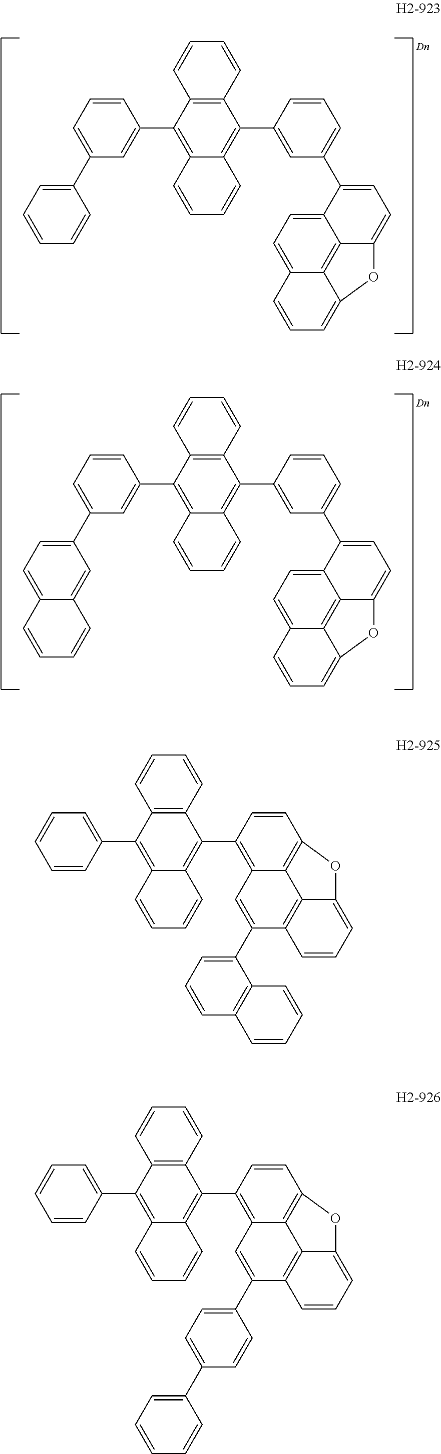

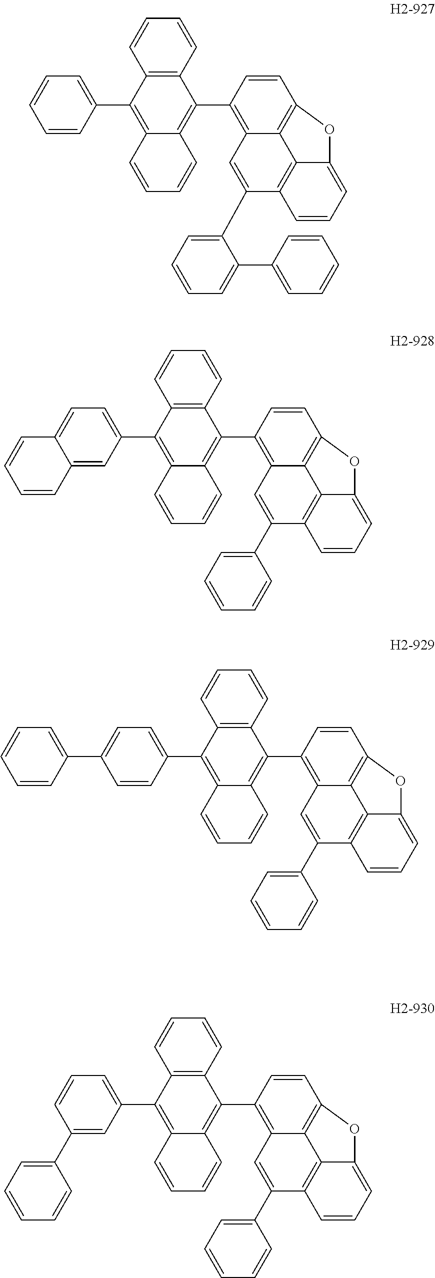 [SMILES: c1ccc(-c2ccccc2-c2cc3c(-c4c5ccccc5c(-c5ccccc5)c5ccccc45)ccc4oc5cccc2c5c43)cc1 ; c1ccc(-c2cc3c(-c4c5ccccc5c(-c5ccc6ccccc6c5)c5ccccc45)ccc4oc5cccc2c5c43)cc1 ; c1ccc(-c2ccc(-c3c4ccccc4c(-c4ccc5oc6cccc7c(-c8ccccc8)cc4c5c67)c4ccccc34)cc2)cc1 ; c1ccc(-c2cccc(-c3c4ccccc4c(-c4ccc5oc6cccc7c(-c8ccccc8)cc4c5c67)c4ccccc34)c2)cc1]

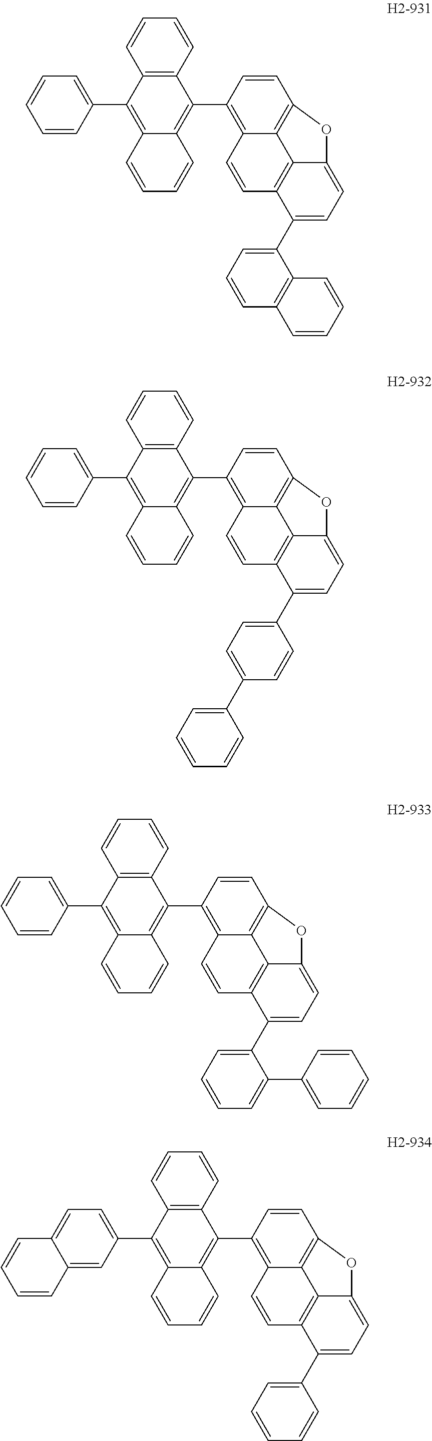 [SMILES: c1ccc(-c2c3ccccc3c(-c3ccc4oc5ccc(-c6cccc7ccccc67)c6ccc3c4c56)c3ccccc23)cc1 ; c1ccc(-c2ccc(-c3ccc4oc5ccc(-c6c7ccccc7c(-c7ccccc7)c7ccccc67)c6ccc3c4c56)cc2)cc1 ; c1ccc(-c2ccccc2-c2ccc3oc4ccc(-c5c6ccccc6c(-c6ccccc6)c6ccccc56)c5ccc2c3c45)cc1 ; c1ccc(-c2ccc3oc4ccc(-c5c6ccccc6c(-c6ccc7ccccc7c6)c6ccccc56)c5ccc2c3c45)cc1]

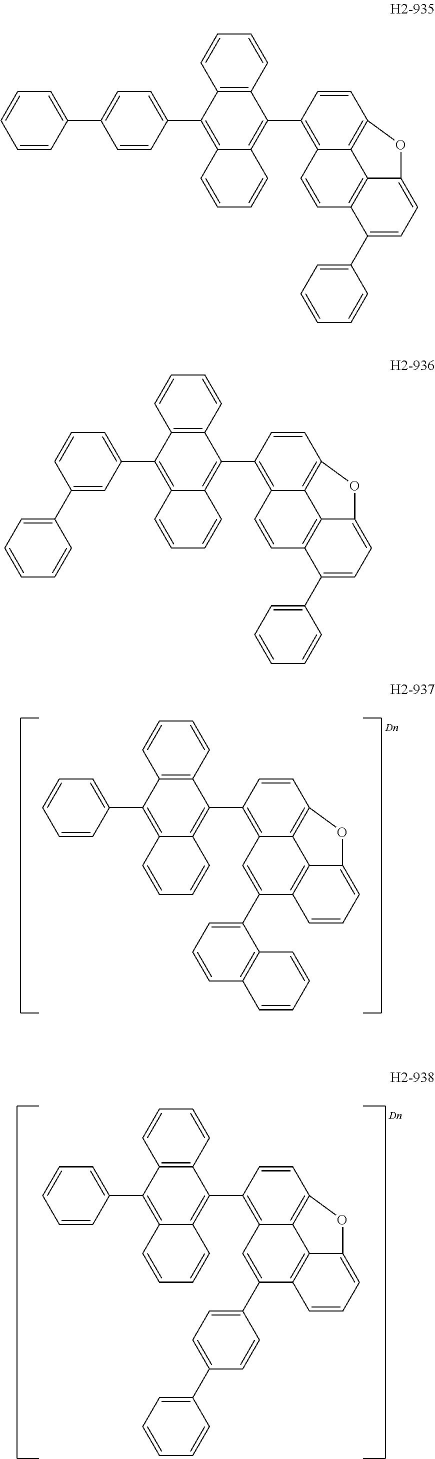

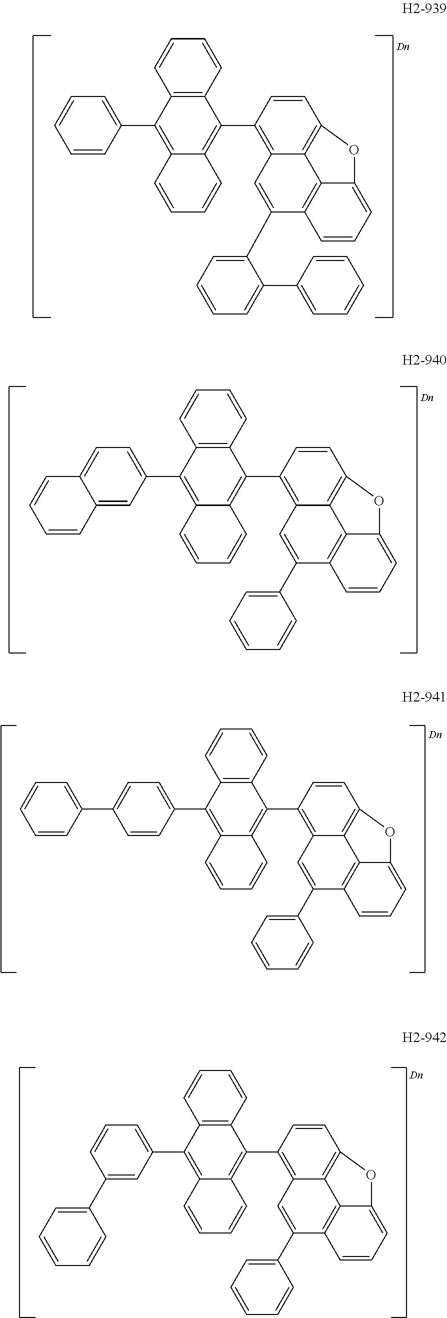

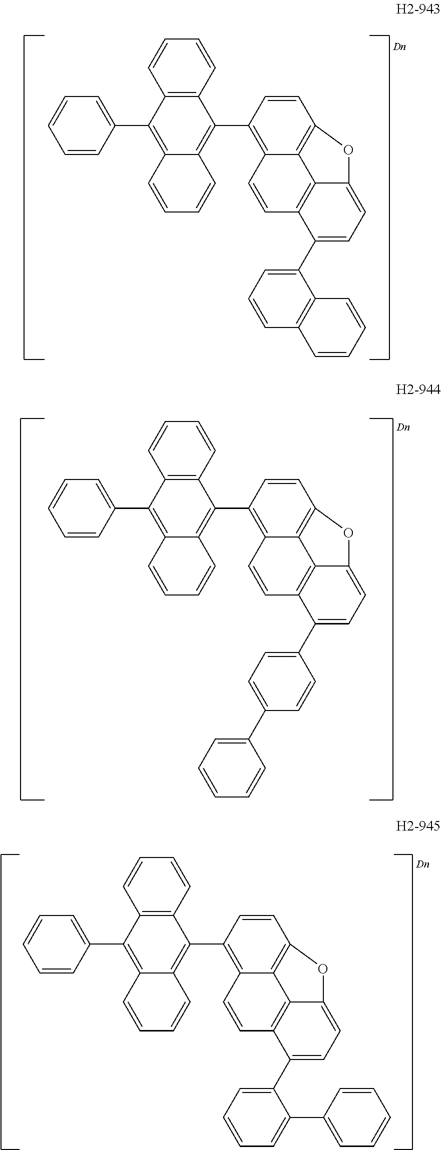

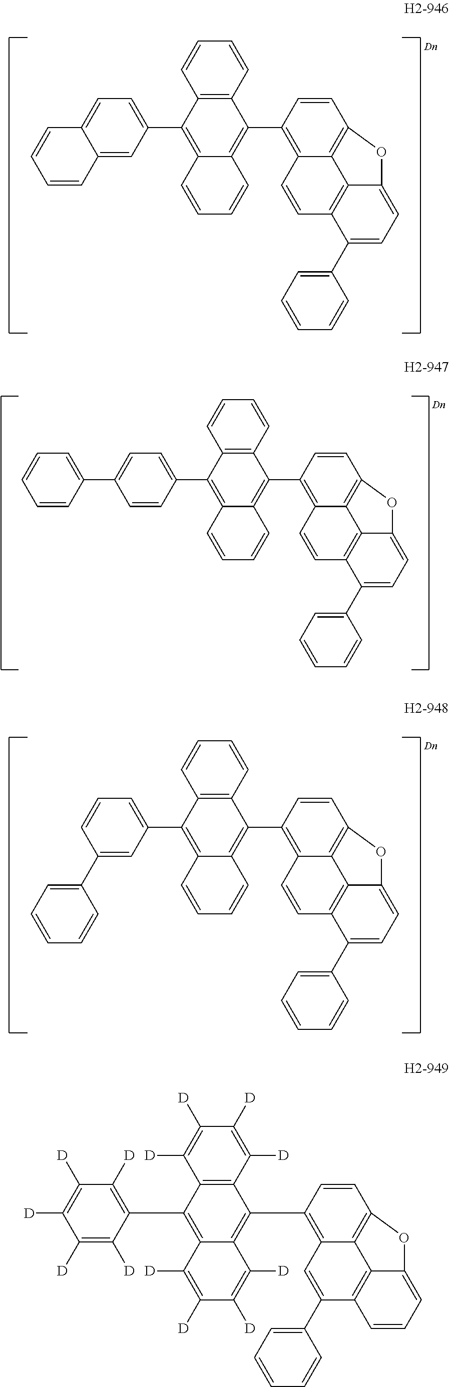

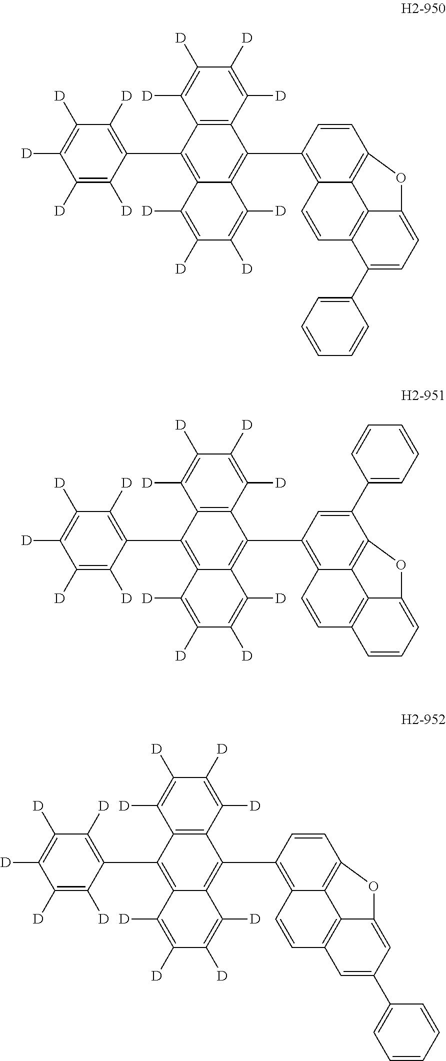 [SMILES: [2H]c1c([2H])c([2H])c(-c2c3c([2H])c([2H])c([2H])c([2H])c3c(-c3ccc4oc5ccc(-c6ccccc6)c6ccc3c4c56)c3c([2H])c([2H])c([2H])c([2H])c23)c([2H])c1[2H] ; [2H]c1c([2H])c([2H])c(-c2c3c([2H])c([2H])c([2H])c([2H])c3c(-c3cc(-c4ccccc4)c4oc5cccc6ccc3c4c65)c3c([2H])c([2H])c([2H])c([2H])c23)c([2H])c1[2H] ; [2H]c1c([2H])c([2H])c(-c2c3c([2H])c([2H])c([2H])c([2H])c3c(-c3ccc4oc5cc(-c6ccccc6)cc6ccc3c4c65)c3c([2H])c([2H])c([2H])c([2H])c23)c([2H])c1[2H]]

In the above compounds, Dₙ means that n hydrogens are substituted with deuterium, and n is an integer from 1 to the maximum number of hydrogens in the compound.

According to one embodiment of the present disclosure, in the plurality of host materials of the present disclosure, at least one of Formulas 1 and 2 comprises deuterium. Preferably, in the plurality of host materials of the present disclosure, Formula 1 may be substituted with deuterium. In addition, in the plurality of host materials of the present disclosure, the deuterium substitution rate of Formula 1 or 2 may be preferably 20% or more and 100% or less, more preferably 20% or more and less than 90%, and even more preferably 50% or more and less than 90%. In addition, preferably, the plurality of host materials of the present disclosure may comprise hydrogen in the formulas that are not substituted with deuterium.

The present disclosure provides the organic electroluminescent compound represented by Formula 1-a.

The organic electroluminescent compound represented by Formula 1-a is described in more detail as follows.

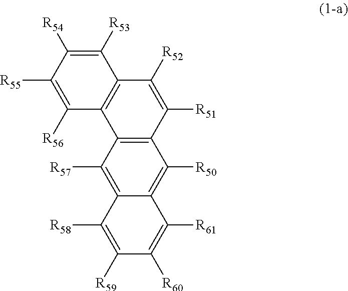

In Formula 1-a, at least one of R₅₀ to R₆₁ represents the following Formula 1-b or the following Formula 1-c, and the remainder are each independently hydrogen or deuterium. For example, R₅₇ may be Formula 1-b or Formula 1-c, and R₅₀ to R₅₆ and R₅₈ to R₆₁ may be hydrogen or deuterium; or R₅₀ may be Formula 1-b or Formula 1-c, and R₅₁ to R₆₁ may be hydrogen or deuterium.

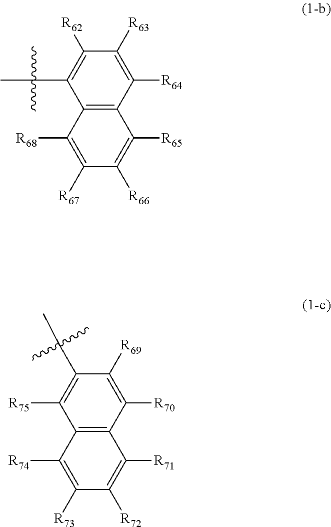

In Formulas 1-b and 1-c, R₆₂ to R₆₅ and R₆₈ each independently represent hydrogen, deuterium, a phenyl unsubstituted or substituted with deuterium, a biphenyl unsubstituted or substituted with deuterium, or a naphthyl unsubstituted or substituted with deuterium, and at least one of R₆₂ to R₆₅ and R₆₈ represents a phenyl unsubstituted or substituted with deuterium, a biphenyl unsubstituted or substituted with deuterium, or a naphthyl unsubstituted or substituted with deuterium.

In Formulas 1-b and 1-c, R₆₆ and R₆₇ each independently represent hydrogen or deuterium.

In Formulas 1-b and 1-c, R₆₉ to R₇₅ each independently represent hydrogen, deuterium, a phenyl unsubstituted or substituted with deuterium, a biphenyl unsubstituted or substituted with deuterium, or a naphthyl unsubstituted or substituted with deuterium, and at least one of R₆₉ to R₇₅ represents a phenyl unsubstituted or substituted with deuterium, a biphenyl unsubstituted or substituted with deuterium, or a naphthyl unsubstituted or substituted with deuterium.

The compound represented by Formula 1-a may be selected from the following compounds, but is not limited thereto.

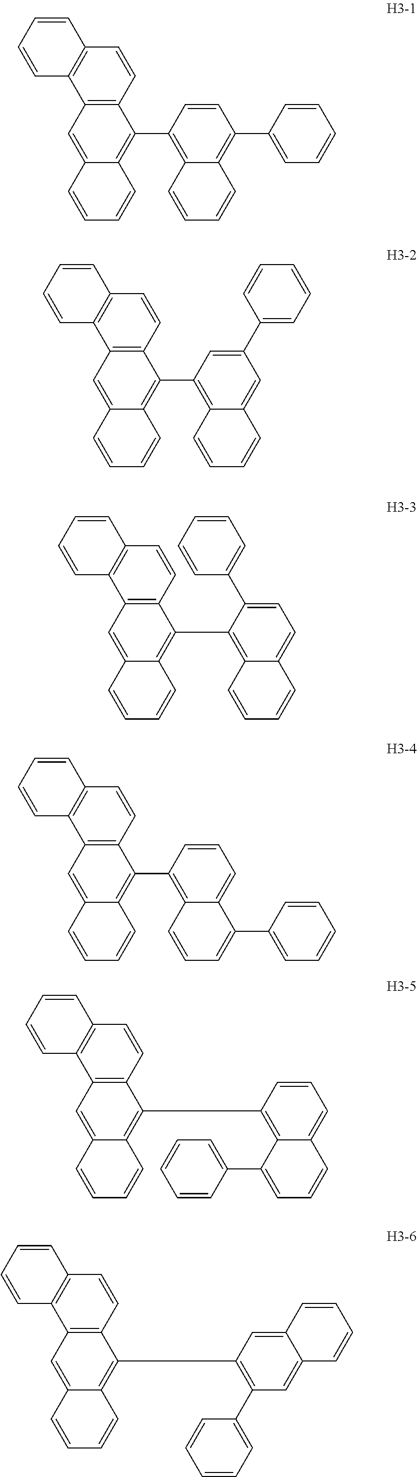

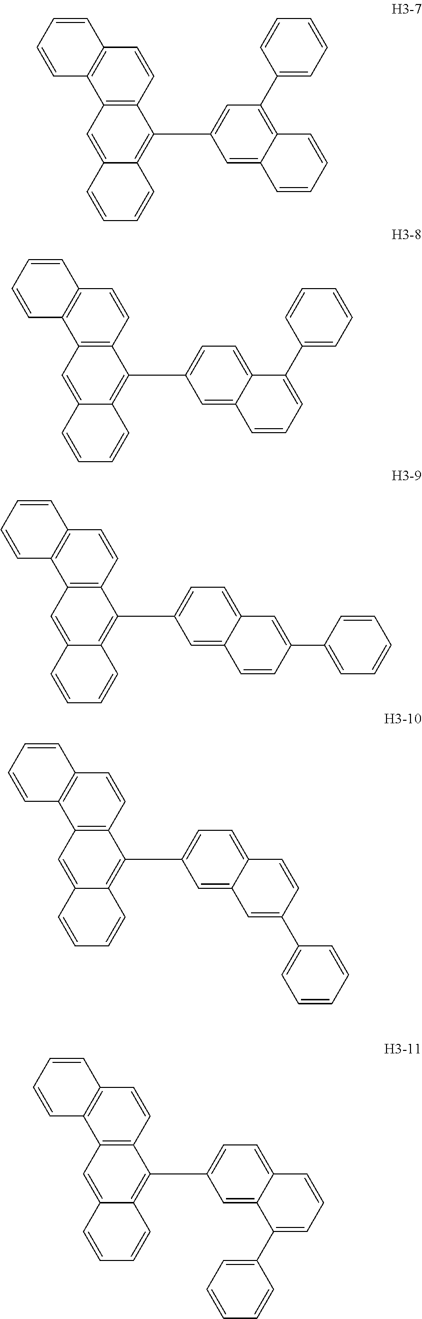

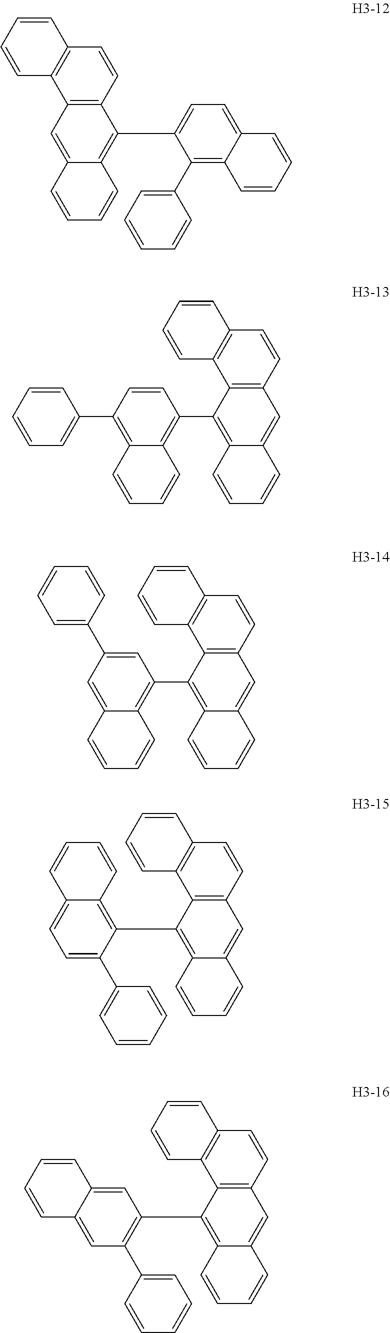

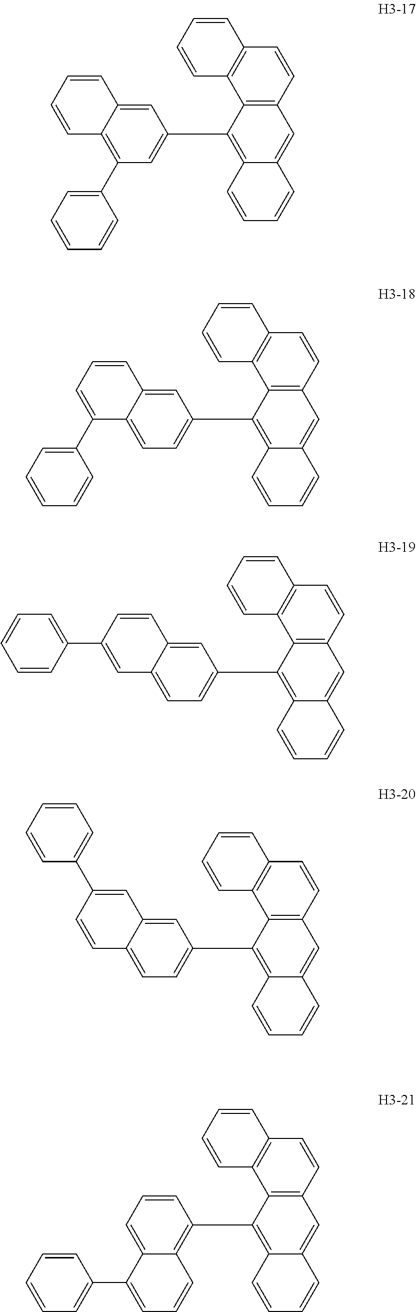

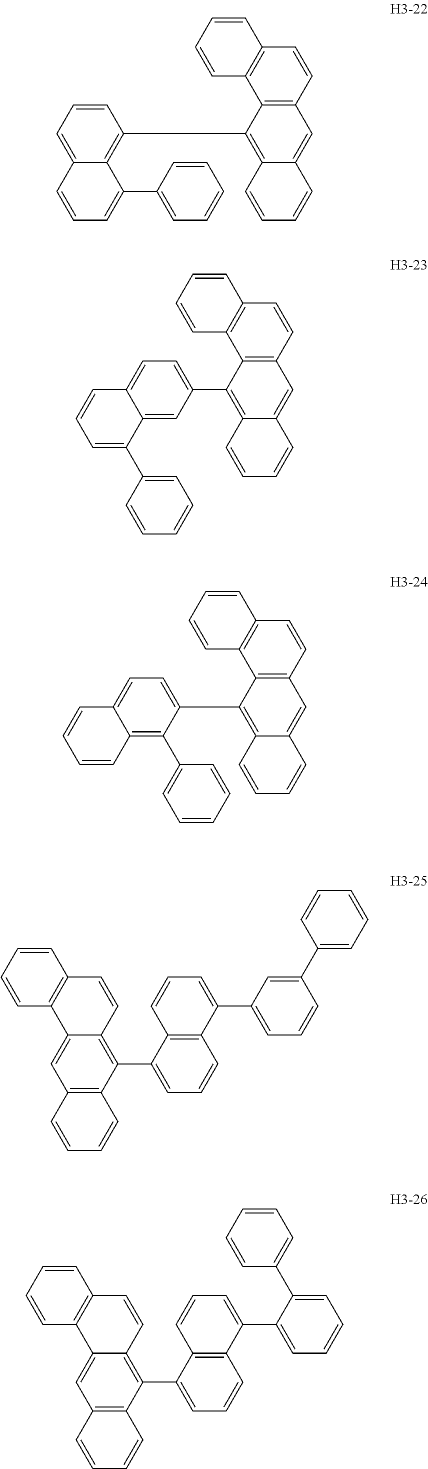

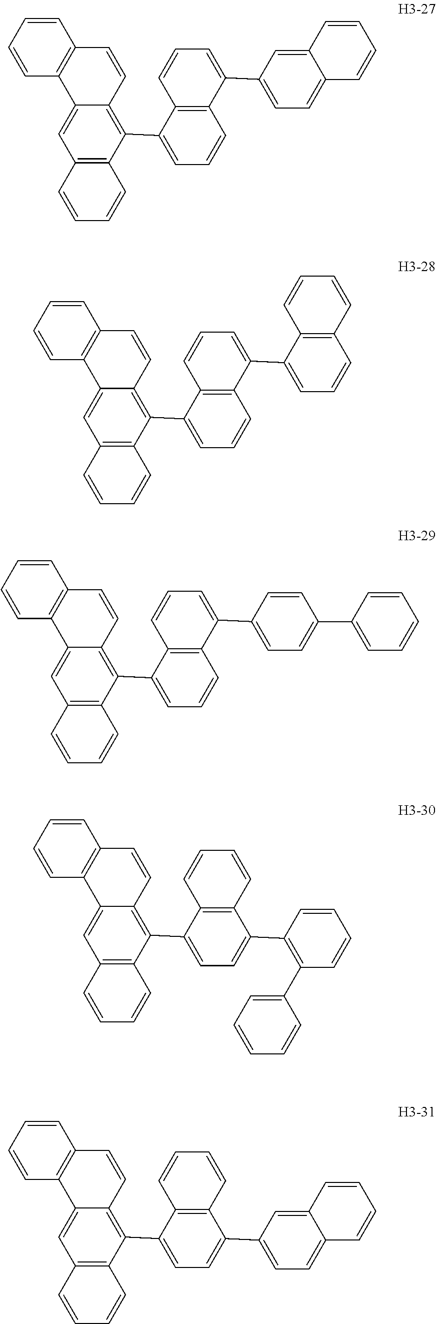

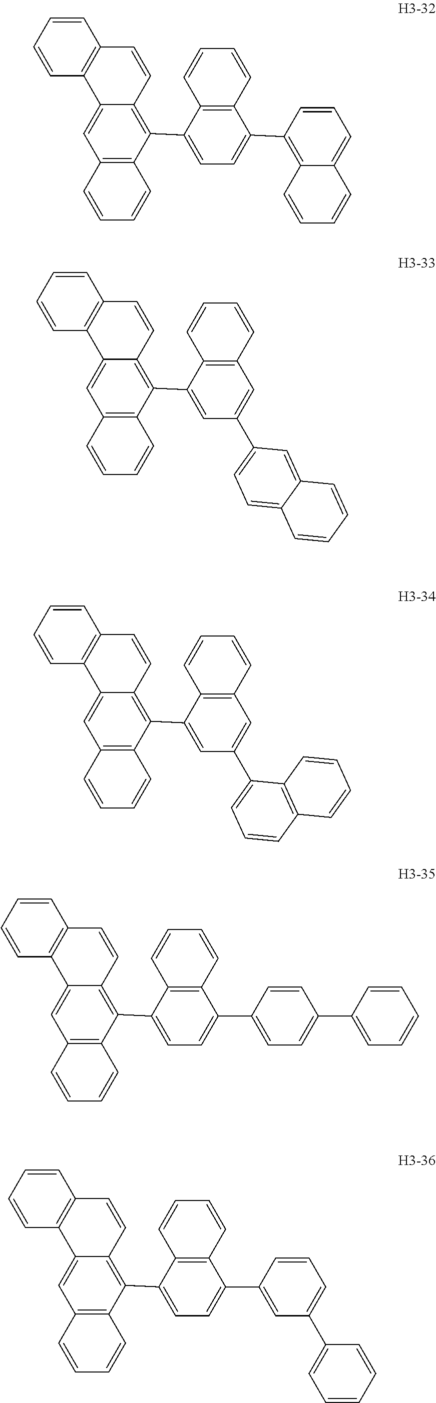

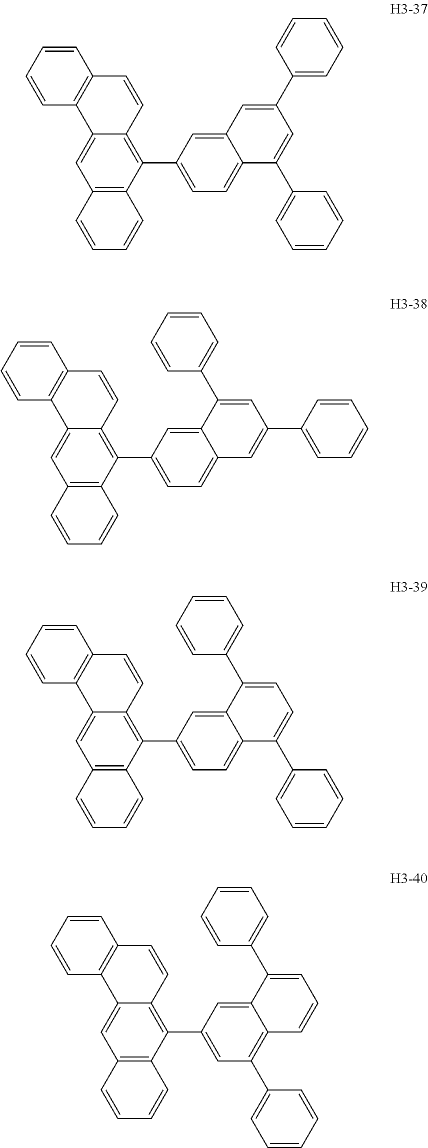

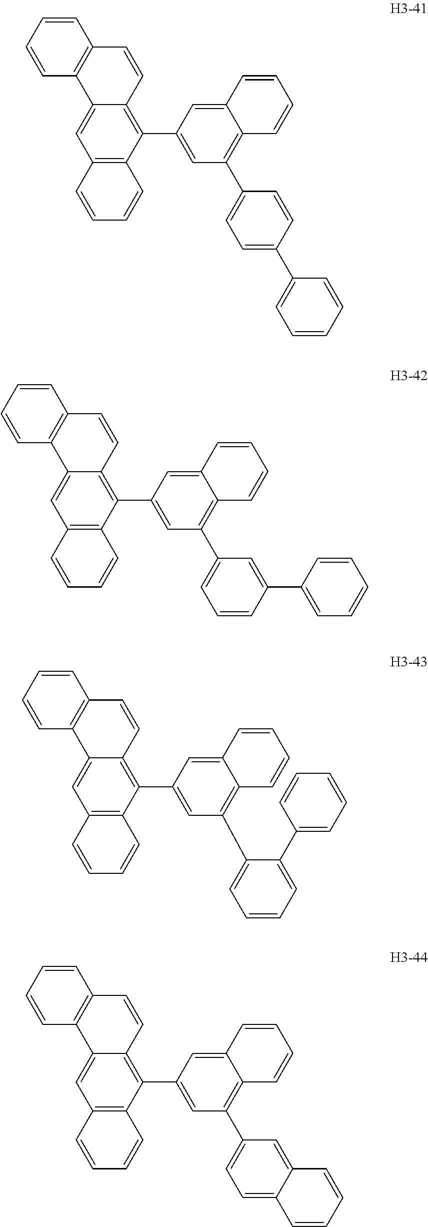

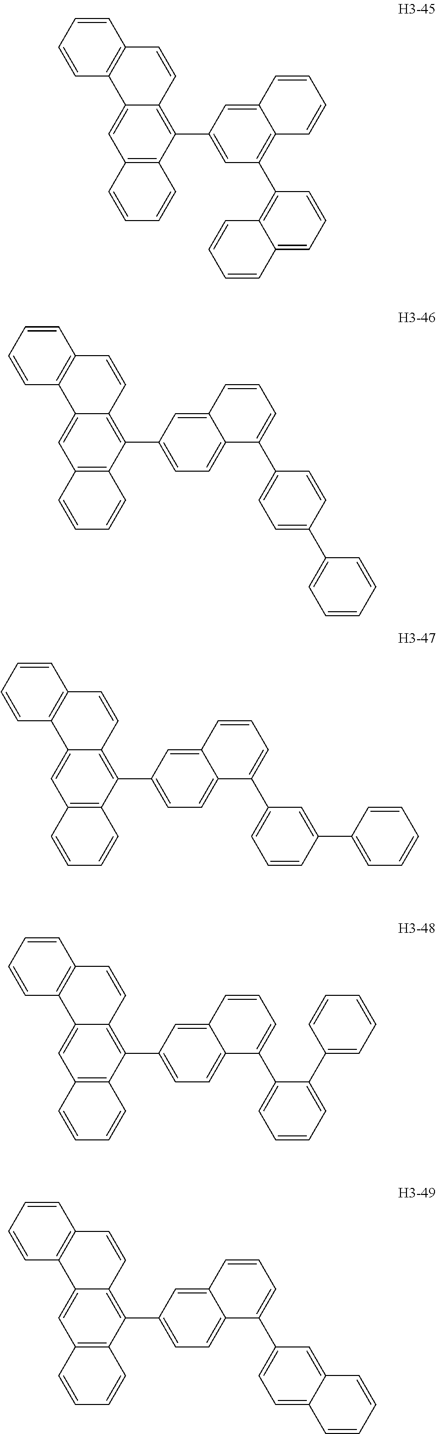

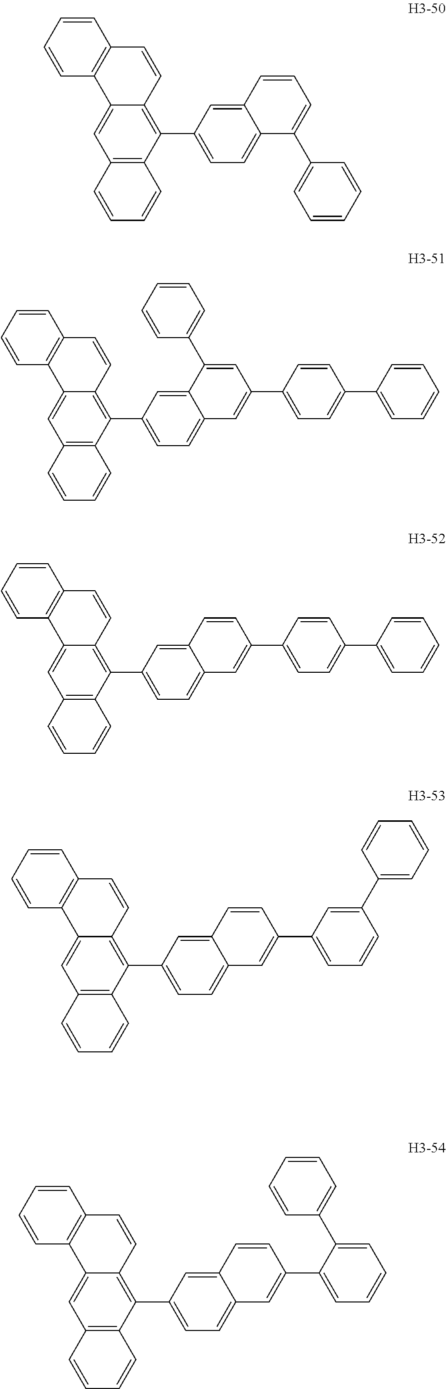

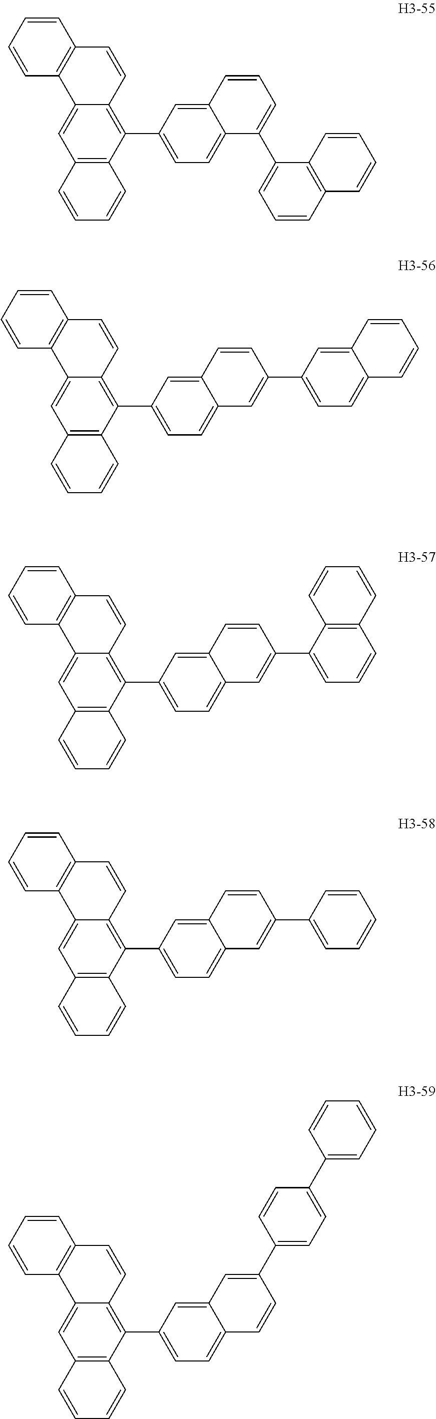

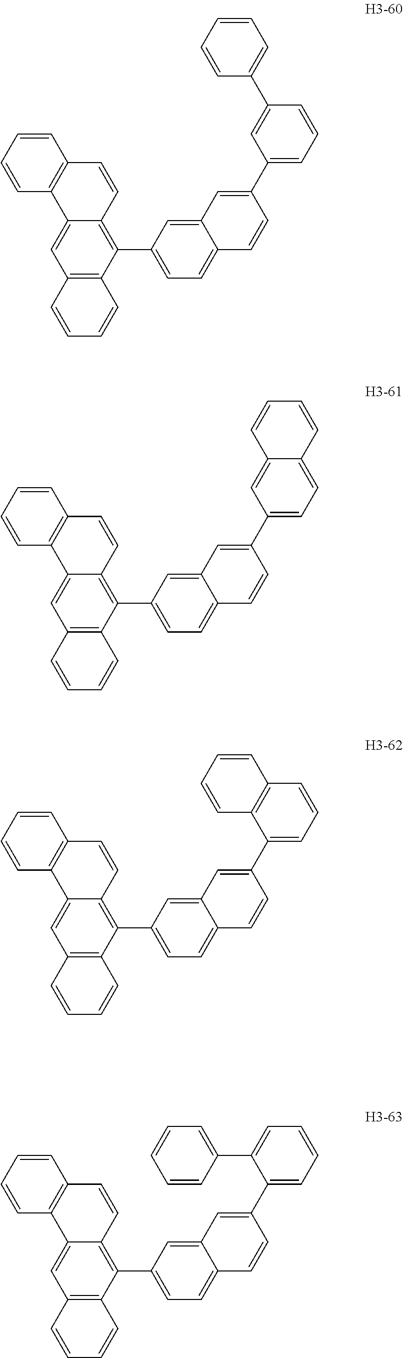

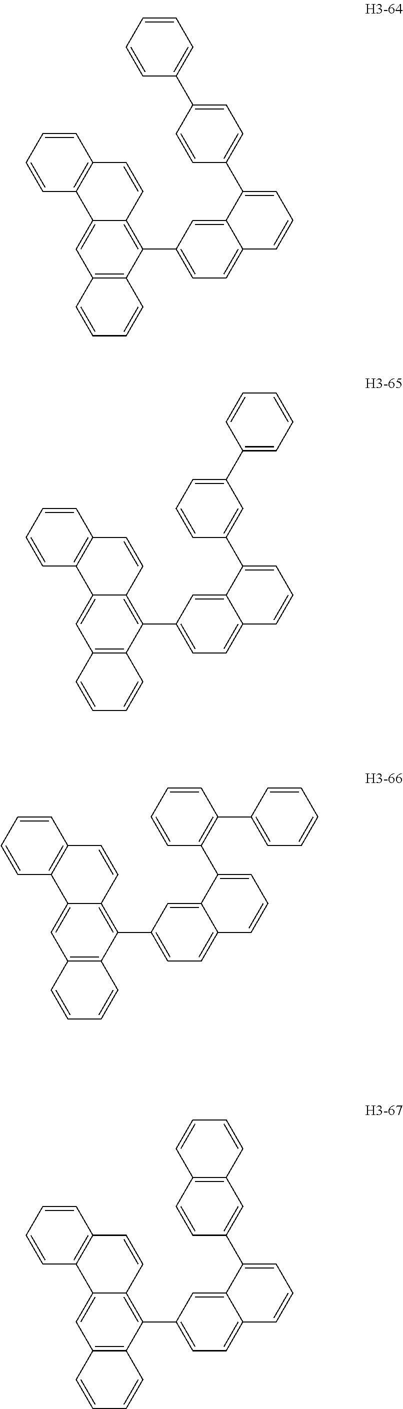

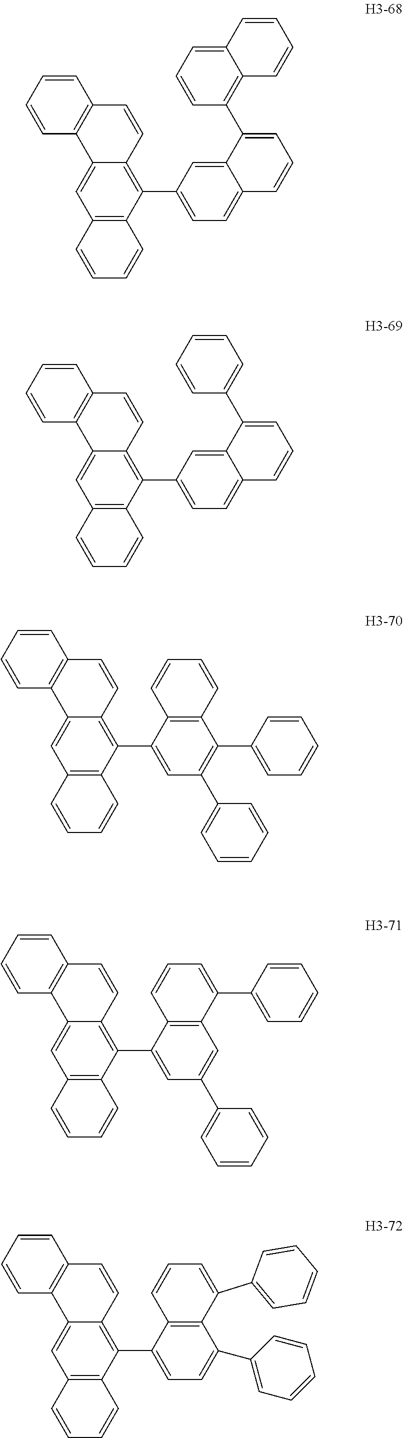

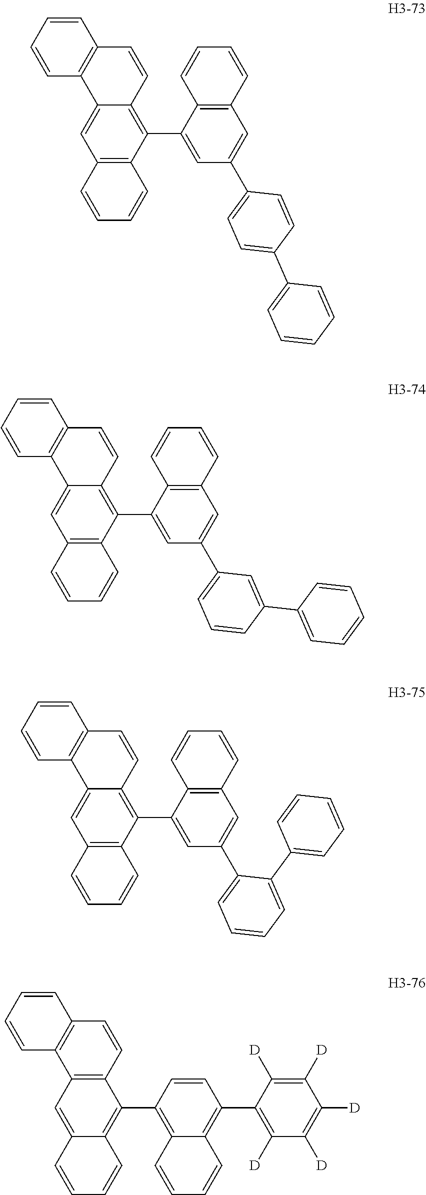

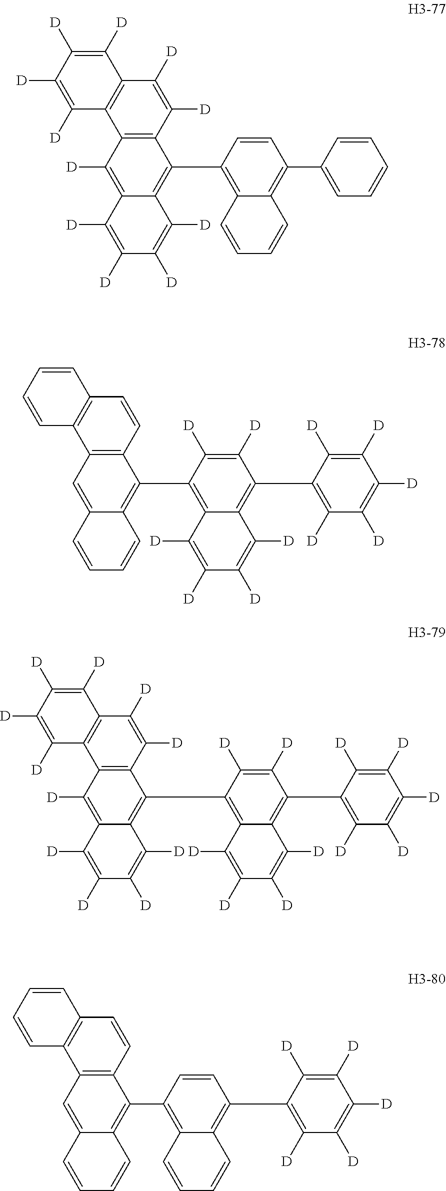

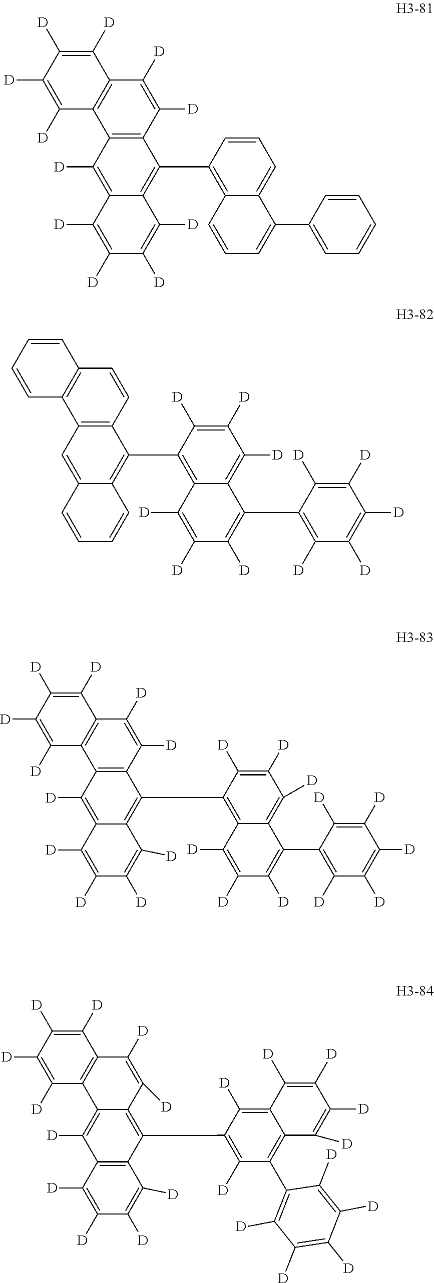

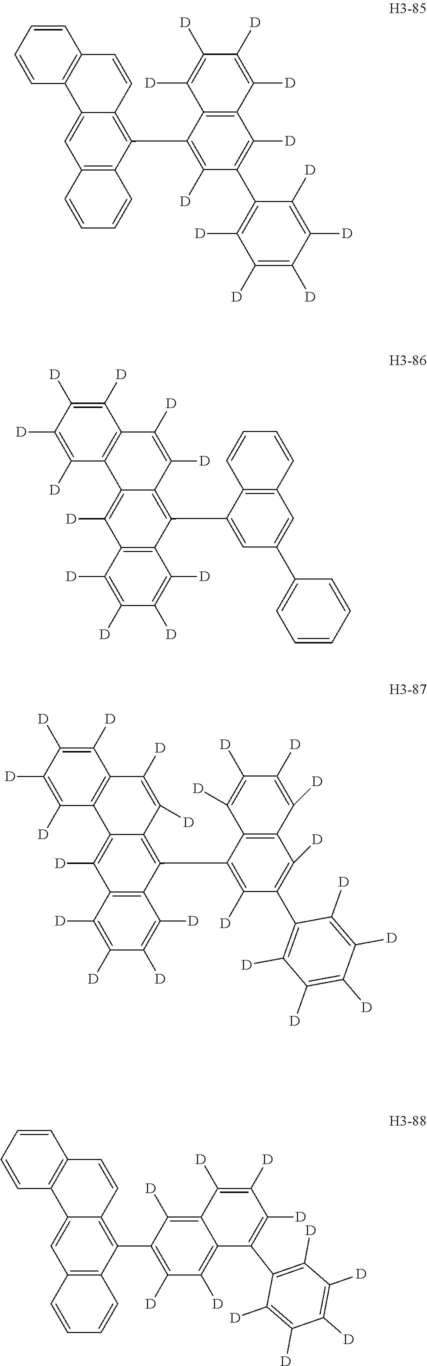

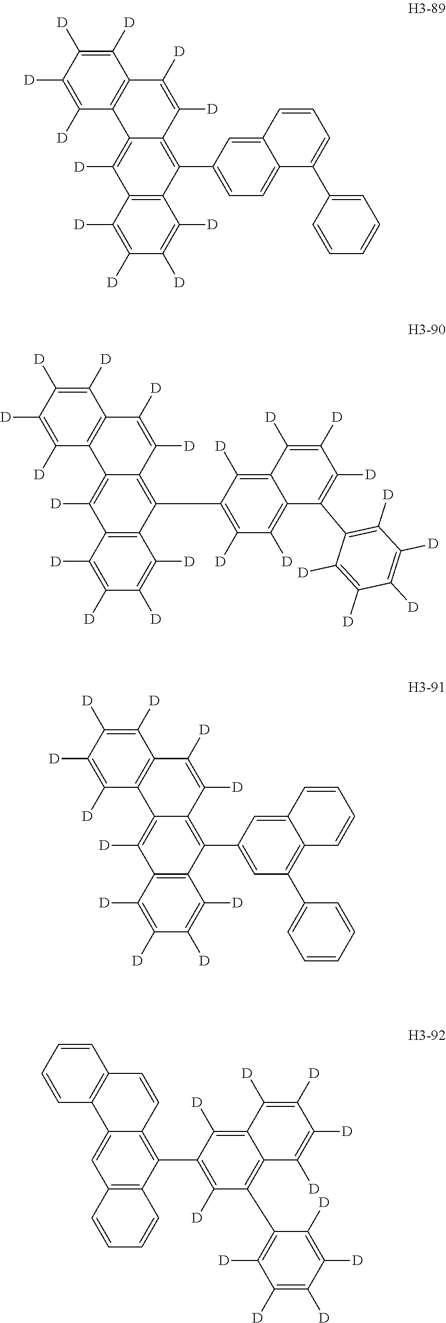

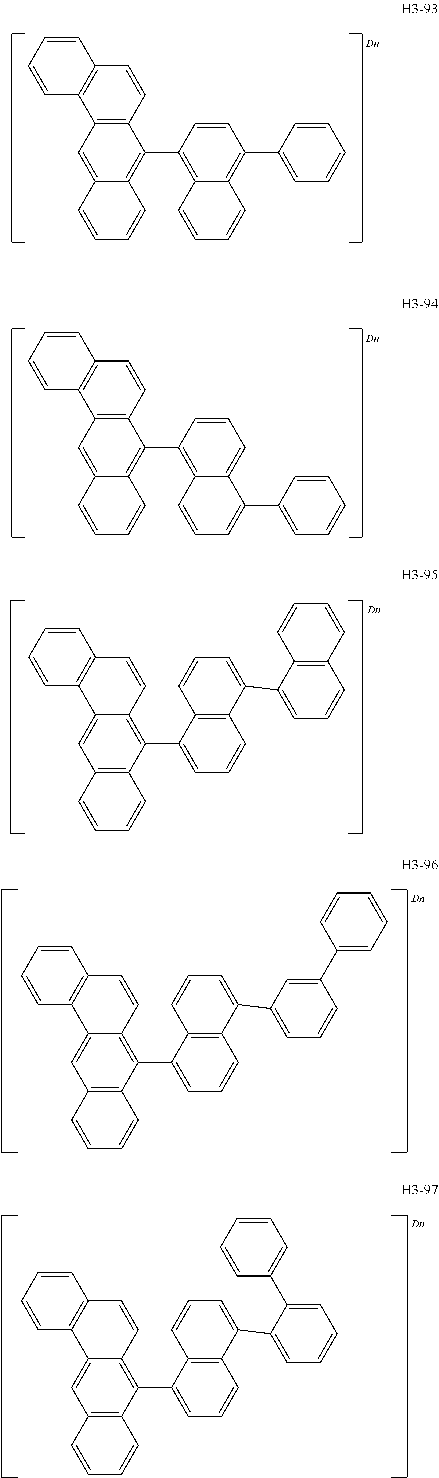

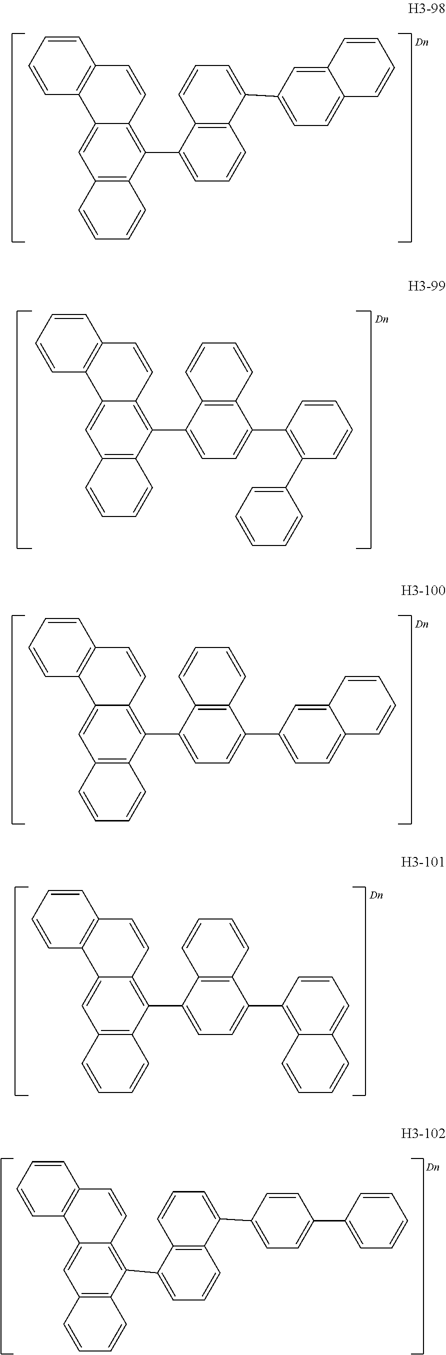

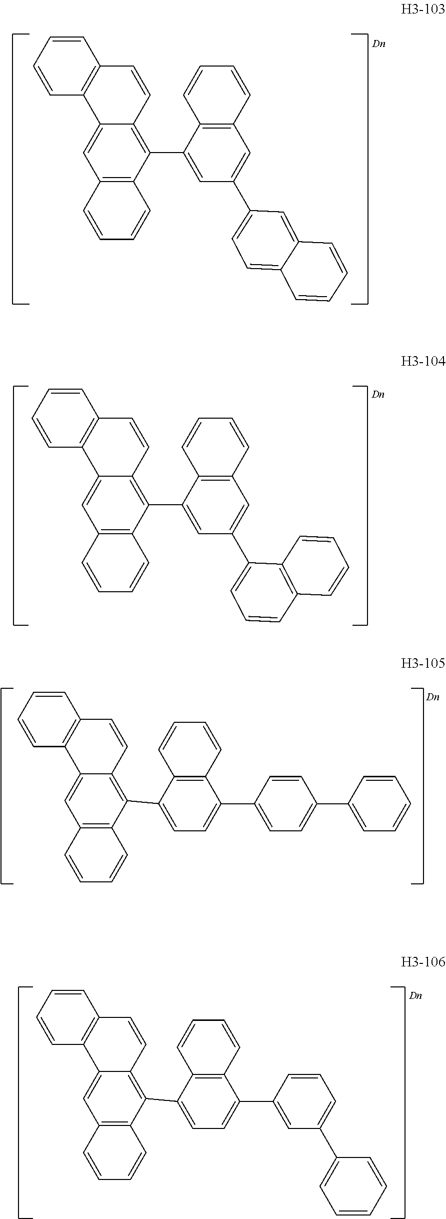

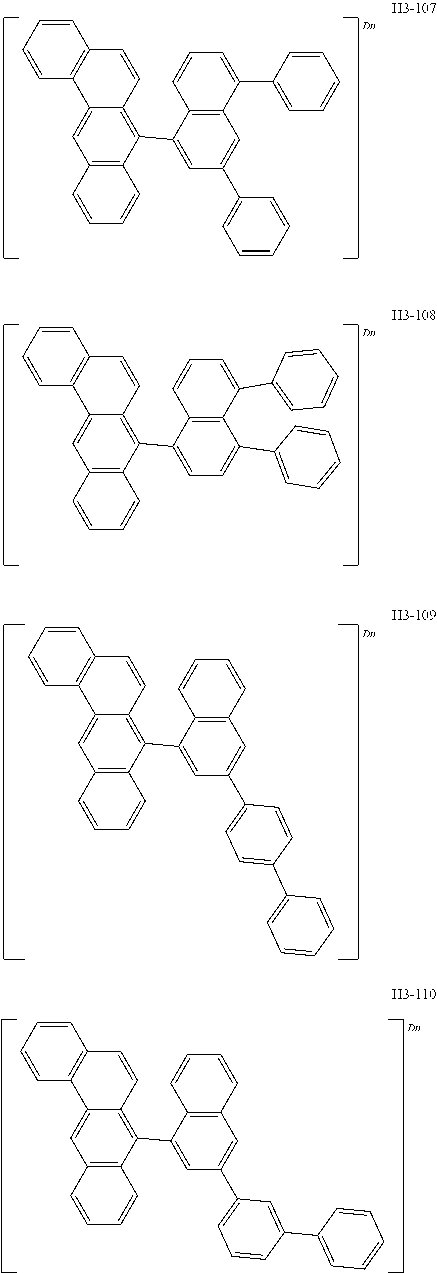

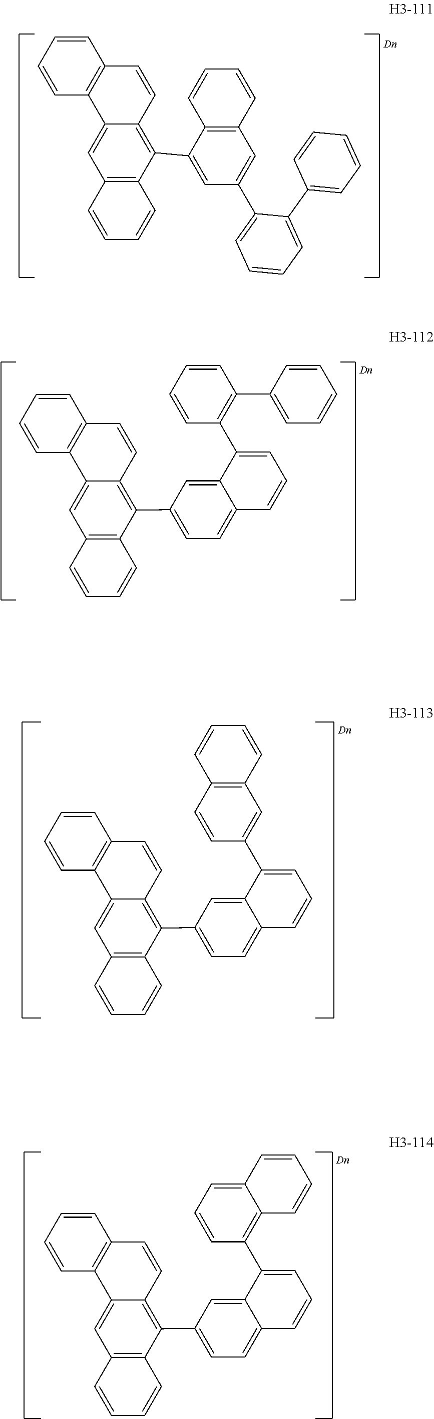

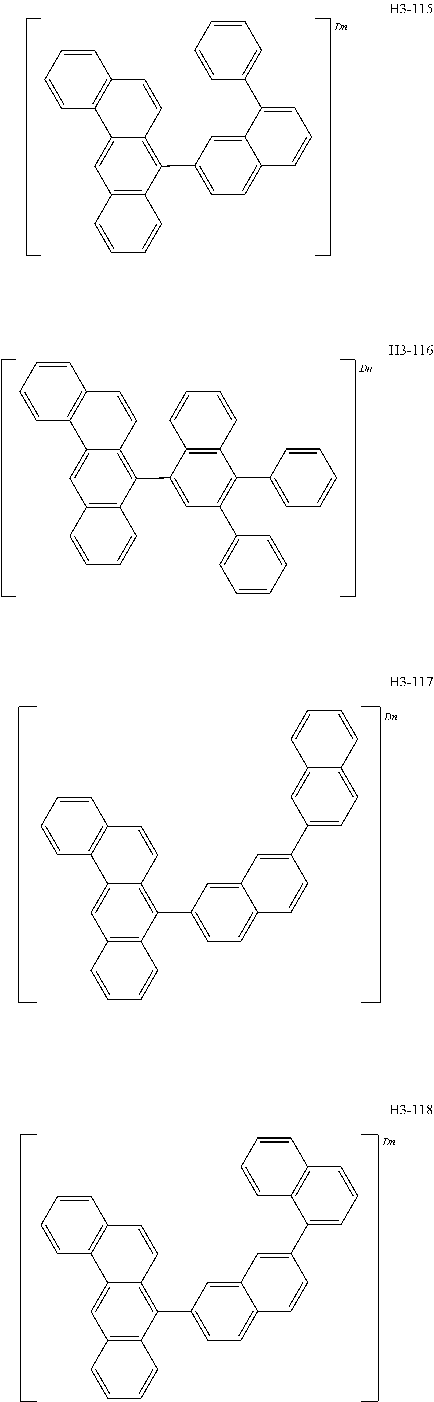

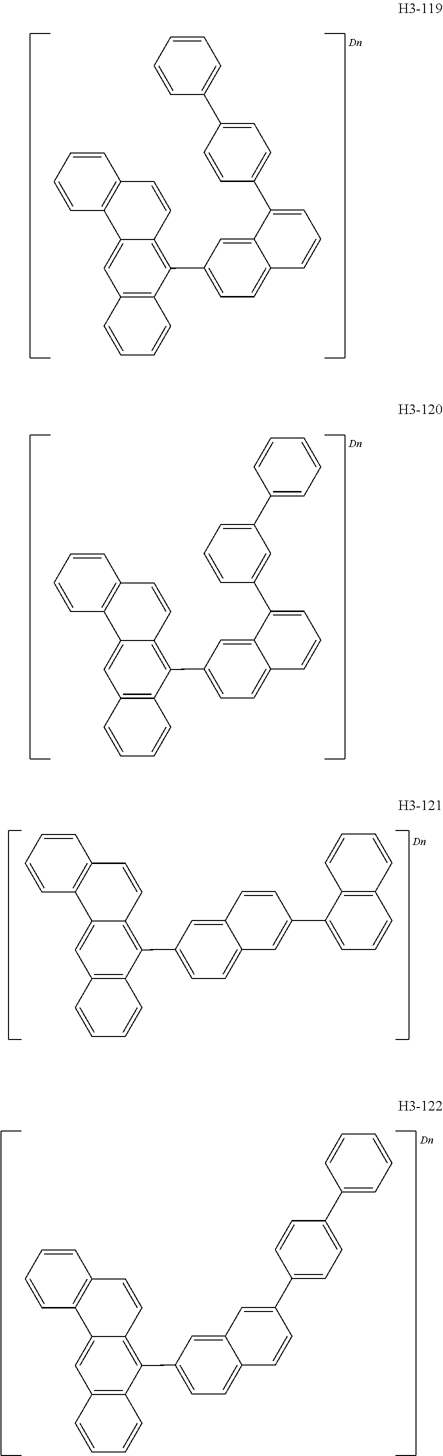

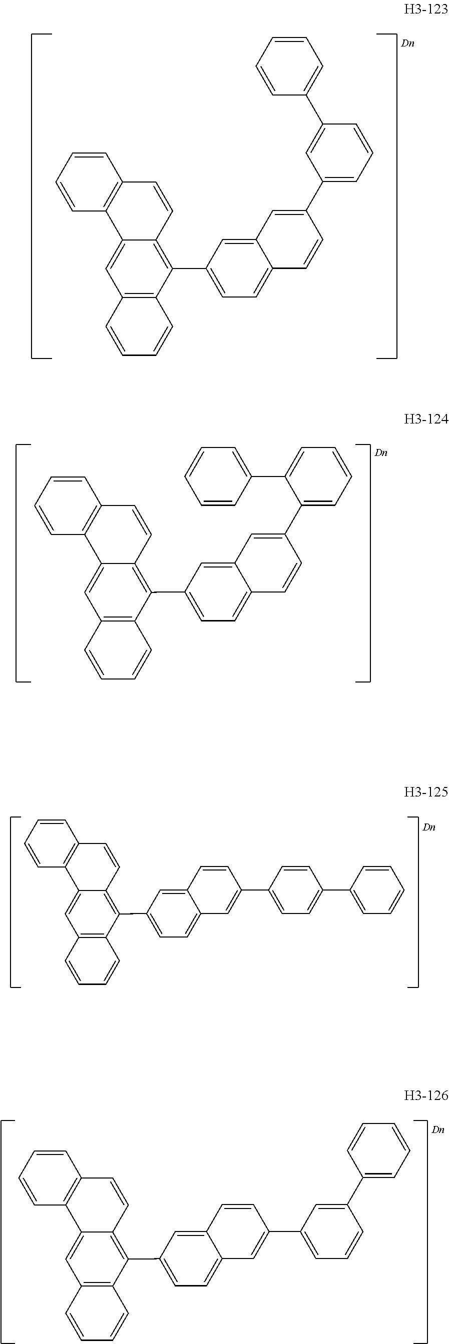

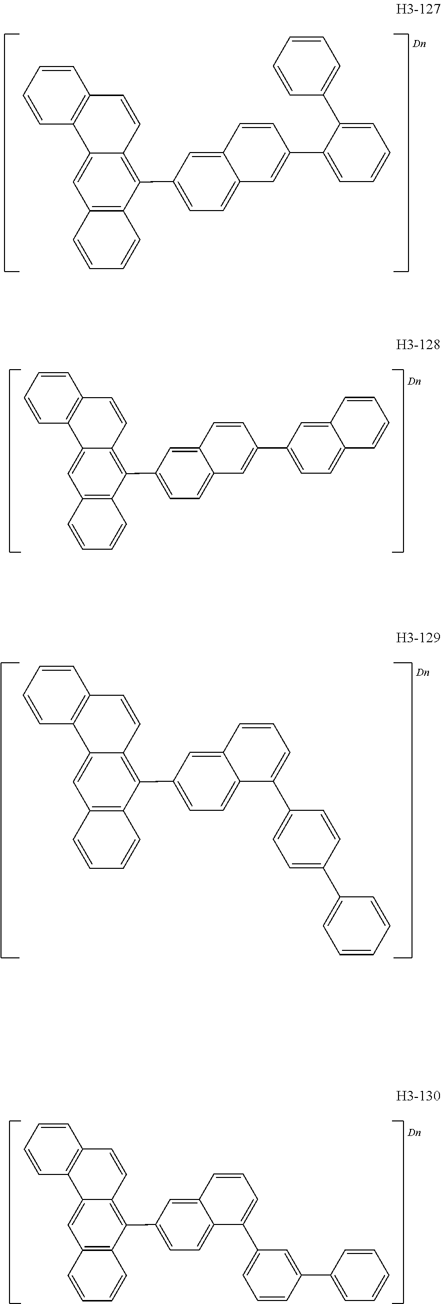

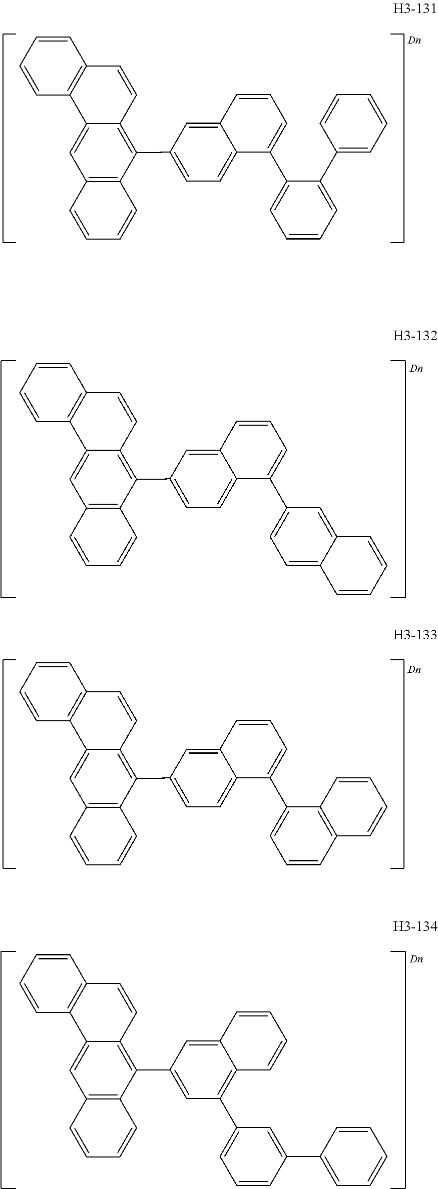

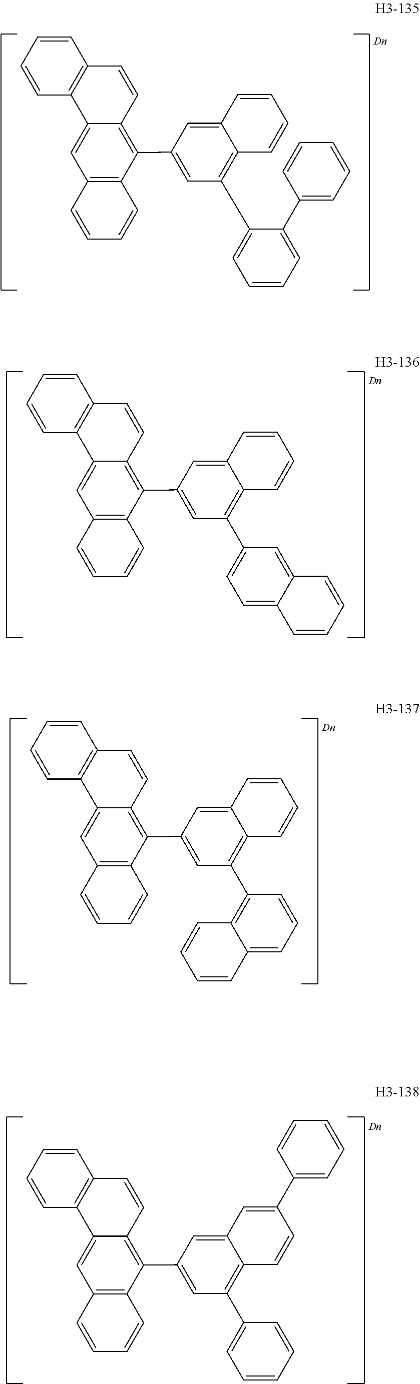

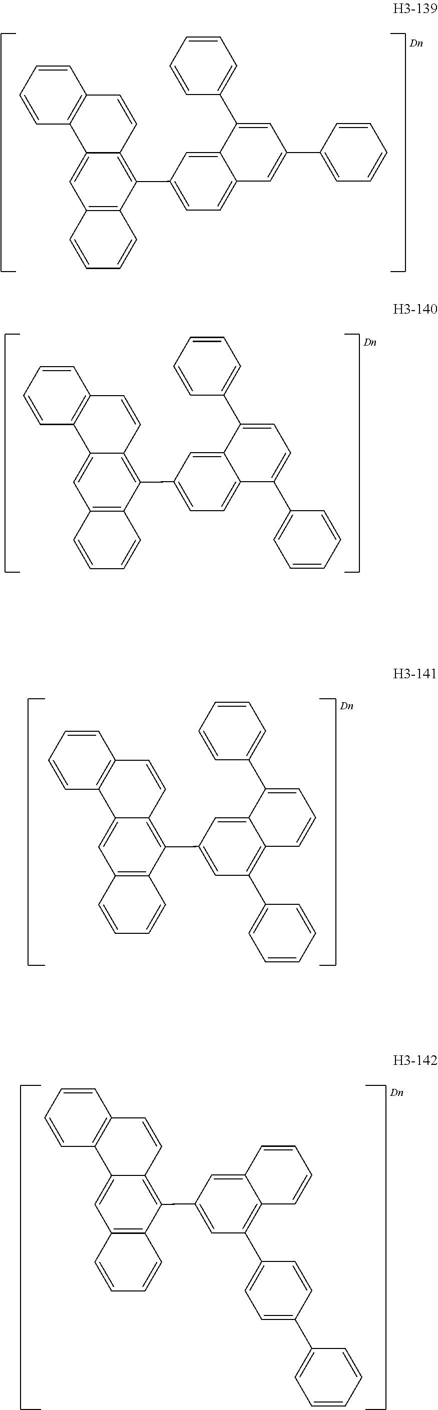

In the above compounds, Dₙ means that n hydrogens are substituted with deuterium, and n is an integer from 1 to the maximum number of hydrogens in the compound.

The present disclosure provides the organic electroluminescent compound represented by Formula 2-a.

The organic electroluminescent compound represented by Formula 2-a is described in more detail as follows.

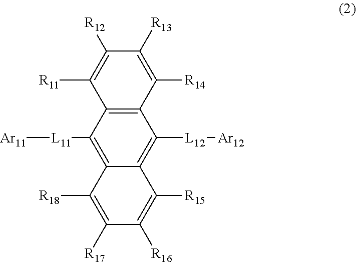

In Formula 2-a, Ar₁₁ represents a substituted or unsubstituted (C₆-C₃₀)aryl or a substituted or unsubstituted (3- to 30-membered)heteroaryl. According to one embodiment of the present disclosure, Ar₁₁ may be a substituted or unsubstituted (C₆-C₂₅)aryl or a substituted or unsubstituted (5- to 25-membered)heteroaryl. According to another embodiment of the present disclosure, Ar₁₁ may be a substituted or unsubstituted (C₆-C₁₈)aryl or a substituted or unsubstituted (5- to 18-membered)heteroaryl. For example, Ar₁₁ may be a phenyl, a biphenyl, or a naphthyl, etc., which may be further substituted with deuterium.

11 represents a single bond, a substituted or unsubstituted (C₆-C₃₀)arylene, or a substituted or unsubstituted (3- to 30-membered)heteroarylene. According to one embodiment of the present disclosure, 11 may be a single bond or a substituted or unsubstituted (C₆-C₂₅)arylene. According to another embodiment of the present disclosure, L₁₁ may be a single bond or a substituted or unsubstituted (C₆-C₁₈)arylene. For example, L₁₁ may be a single bond or a phenylene, etc., which may be further substituted with deuterium.

R₁ to R₁₈ each independently represent hydrogen or deuterium.

L₁₂ represents a single bond or a substituted or unsubstituted (C₆-C₃₀)arylene. According to one embodiment of the present disclosure, L₁₂ may be a single bond.

Ar₁₂ is represented by the following Formula 2-b.

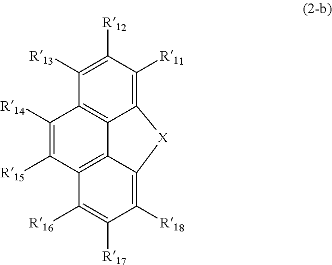

In Formula 2-b, X is O or S.

In Formula 2-b, R′₁₁ to R′₁₈ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a combination thereof; or may be linked to the adjacent substituents to form a ring(s). According to one embodiment of the present disclosure, R′₁₁ to R′₁₈ may each independently be hydrogen, deuterium, or a substituted or unsubstituted (C₆-C₃₀)aryl; or may be linked to the adjacent substituents to form a ring(s). For example, R′₁₁ to R′₁₈ may each independently be hydrogen, deuterium, a phenyl, a biphenyl, or a naphthyl, etc., which may be further substituted with deuterium.

One of R′₁₁ to R′₁₃ is linked to L₁₂, and at least one of the remainder of R′₁₁ to R′₁₃ not linked to L₁₂ and R′₁₄ to R′₁₈ is a substituted or unsubstituted (C₁-C₃₀)alkyl, a substituted or unsubstituted (C₃-C₃₀)cycloalkyl, a substituted or unsubstituted (C₆-C₃₀)aryl, a substituted or unsubstituted (C₆-C₃₀)heteroaryl, or a combination thereof. According to one embodiment of the present disclosure, at least one of the remainder of R′₁₁ to R′₁₃ not linked to L₁₂ and R′₁₄ to R′₁₈ may be a substituted or unsubstituted (C₆-C₃₀)aryl, preferably a (C₆-C₂₅)aryl unsubstituted or substituted with deuterium, and more preferably a (C₆-C₁₈)aryl unsubstituted or substituted with deuterium. For example, R′₁₃ is linked to L₁₂, and at least one of R′₁₁, R′₁₂, and R′₁₄ to R′₁₈ may be a phenyl unsubstituted or substituted with deuterium, a biphenyl unsubstituted or substituted with deuterium, or a naphthyl unsubstituted or substituted with deuterium.

According to one embodiment of the present disclosure, the compound represented by Formula 2-a may be selected from the following compounds, but is not limited thereto.

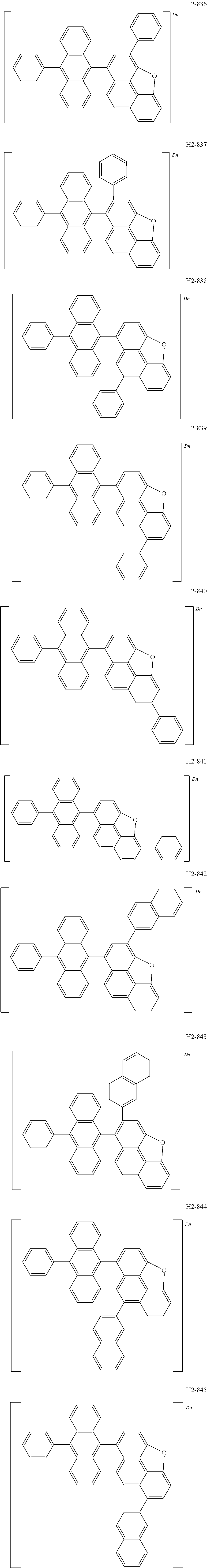

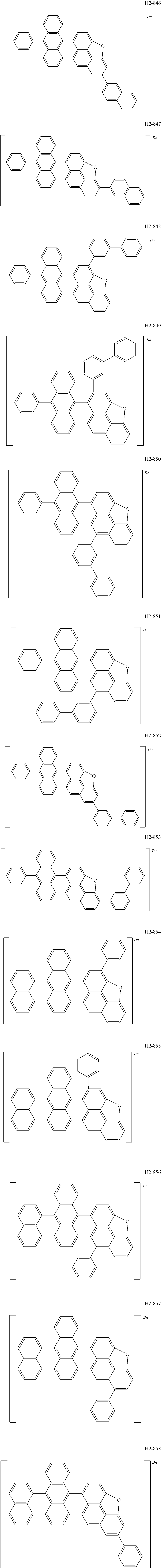

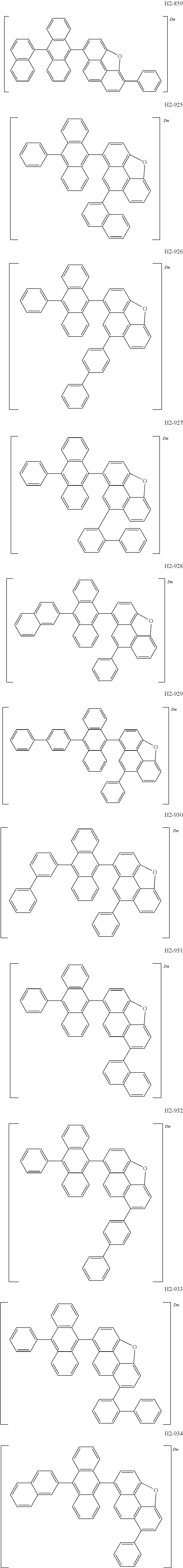

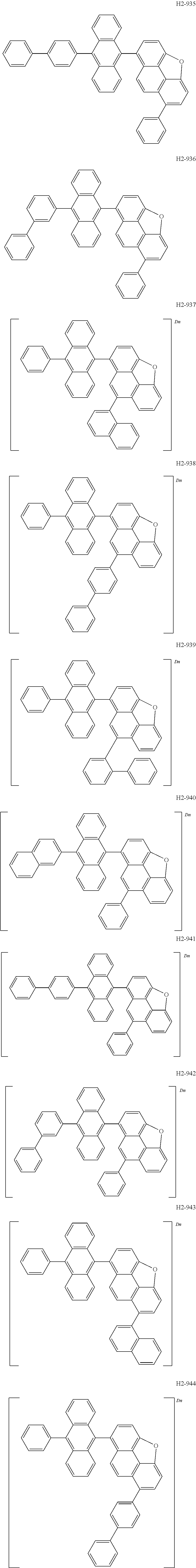

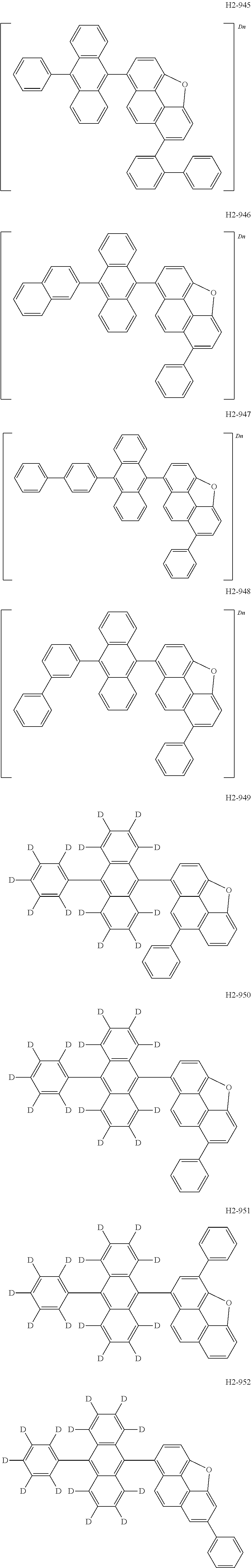

In the above compounds, in Dₙ, n means the number of substituted deuterium, and n means a number from 0 to the maximum number of deuterium in the compound, where n being 0 means a hydrogen compound.

According to one embodiment of the present disclosure, the organic electroluminescent compound represented by Formula 2-a of the present disclosure may comprise deuterium. Preferably, the organic electroluminescent compound represented by Formula 2-a may be partially substituted with deuterium, or entirely substituted with deuterium. For example, it is not necessary for all hydrogens in the organic electroluminescent compound represented by Formula 2-a to be substituted with deuterium. According to another embodiment of the present disclosure, in the organic electroluminescent compound represented by Formula 2-a of the present disclosure, only anthracene may be fully substituted with deuterium, or only -L₁₁-Ar₁₁ may be substituted with deuterium. According to yet another embodiment of the present disclosure, in the organic electroluminescent compound represented by Formula 2-a of the present disclosure, both anthracene and -11-Ar₁₁ may be fully substituted with deuterium.

In the organic electroluminescent compound represented by Formula 2-a, the deuterium substitution rate is preferably 40% or more, more preferably 50% or more, and even more preferably 60% or more of the total number of hydrogens, and for example, the deuterium substitution rate in the formula does not need to be 100%.

The compounds represented by Formulas 1, 1-a, 2, and 2-a according to the present disclosure may be synthesized by referring to synthetic methods known to one skilled in the art. For example, the compound represented by Formula 1 according to the present disclosure may be synthesized by referring to synthetic methods disclosed in Korean Patent Application Laid-Open No. 2021-0046437, etc., but is not limited thereto. The compound represented by Formula 2 according to the present disclosure may be synthesized by referring to synthetic methods disclosed in Korean Patent Application Laid-Open No. 2022-0012180, etc., but is not limited thereto. Among the compounds represented by Formulas 1 and 2 of the present disclosure, compounds substituted with deuterium may be produced by referring to the above literature as well as Korean Patent Application Laid-Open No. 2012-0101029, but are not limited thereto.

In the case where the hydrogen atoms present in the compound according to the present disclosure are substituted with deuterium, hydrogens at various positions within the compound may be replaced by deuterium. The compound substituted with deuterium may be a composition comprising two or more isotopes having different molecular weights depending on the number of substituted deuterium, and an isotope having the highest isotope content for each mass number may be present within the composition. Preferably, the isotope with the highest isotope content for each mass number appears at 50% or more of the number of hydrogens present in the compound, and the substitution distribution of deuterium may vary within the numerical range. The substitution distribution of deuterium can be obtained through mass chromatogram analysis.

The organic electroluminescent device according to the present disclosure comprises an anode, a cathode, and at least one light-emitting layer between the anode and the cathode. At least one layer of the light-emitting layers comprises a plurality of host materials comprising at least one first host compound and at least one second host compound, wherein the first host compound is represented by Formula 1, the second host compound is represented by Formula 2, and the first host compound and the second host compound comprise different kinds of host materials. Here, the weight ratio of the first host compound to the second host compound in the light-emitting layer may be in a range of about 1:99 to about 99:1, preferably about 10:90 to about 90:10, more preferably about 30:70 to about 70:30, more preferably about 40:60 to about 60:40, and even more preferably about 50:50. For example, the plurality of host materials of the present disclosure may include at least one compound among the first host compounds H1-1 to H1-370 and at least one compound among the second host compounds H2-1 to H2-952, and this plurality of host materials may be included in the same organic layer, for example, the light-emitting layer, or may each be included in different light-emitting layers.

The organic electroluminescent compound represented by Formula 1-a or Formula 2-a of the present disclosure may be included in at least one layer of a light-emitting layer, a hole injection layer, a hole transport layer, a hole auxiliary layer, a light-emitting auxiliary layer, an electron transport layer, an electron buffer layer, an electron injection layer, an interlayer, a hole-blocking layer, and an electron-blocking layer; and, in some cases, preferably may be included in at least one layer of a light-emitting layer, a hole transport layer, a hole auxiliary layer, a light-emitting auxiliary layer, an electron transport layer, an electron buffer layer, a hole-blocking layer, and an electron-blocking layer. When included in a light-emitting layer, the organic electroluminescent compound represented by Formula 1-a or Formula 2-a of the present disclosure may be included as a host material.

According to one embodiment of the present disclosure, the organic electroluminescent device of the present disclosure comprises an anode, a cathode, a first light-emitting layer disposed between the anode and the cathode, and a second light-emitting layer disposed between the first light-emitting layer and the cathode. The first light-emitting layer comprises the organic electroluminescent compound represented by Formula 1-a, the second light-emitting layer comprises at least two different compounds comprising an anthracene skeleton, and the first light-emitting layer and the second light-emitting layer are in direct contact. According to another embodiment of the present disclosure, the organic electroluminescent device of the present disclosure comprises an anode, a cathode, a first light-emitting layer disposed between the anode and the cathode, and a second light-emitting layer disposed between the first light-emitting layer and the cathode. The second light-emitting layer comprises the organic electroluminescent compound represented by Formula 2-a, and the first light-emitting layer and the second light-emitting layer are in direct contact. Preferably, the second light-emitting layer may further comprise an additional anthracene-based compound in addition to the organic electroluminescent compound of Formula 2-a of the present disclosure.

In addition to the hole transport layer and the light-emitting layer, the organic layer may further include at least one layer selected from a hole injection layer, a hole auxiliary layer, a light-emitting auxiliary layer, an electron transport layer, an electron injection layer, an interlayer, a hole-blocking layer, an electron-blocking layer, and an electron buffer layer. The organic layer may further comprise an amine-based compound and/or an azine-based compound in addition to the light-emitting material of the present disclosure. Specifically, the hole injection layer, the hole transport layer, the hole auxiliary layer, the light-emitting layer, the light-emitting auxiliary layer, or the electron-blocking layer may comprise an amine-based compound, for example, an arylamine-based compound, a styrylarylamine-based compound, etc., as a hole injection material, a hole transport material, a hole auxiliary material, a light-emitting material, a light-emitting auxiliary material, or an electron-blocking material. In addition, the electron transport layer, the electron injection layer, the electron buffer layer, or the hole-blocking layer may comprise an azine-based compound as an electron transport material, an electron injection material, an electron buffer material, or a hole-blocking material. In addition, the organic layer may further comprise at least one metal selected from the group consisting of metals of Group 1, metals of Group 2, transition metals of the 4^{th} period, transition metals of the 5^{th} period, lanthanides, and organometallic of the d-transition elements of the Periodic Table, or at least one complex compound comprising the metal thereof.

The anode and the cathode may each be formed of a transparent conductive material or a semi-transparent or reflective conductive material. Depending on the type of material forming the first electrode and second electrode, the organic electroluminescent device may be a top emission type, a bottom emission type, or a dual-side emission type.

A hole injection layer, a hole transport layer, an electron-blocking layer, or a combination thereof may be used between the anode and the light-emitting layer. The hole injection layer may be multiple layers in order to lower the hole injection barrier (or hole injection voltage) from the anode to the hole transport layer or electron-blocking layer, wherein each layer may use two compounds simultaneously. In addition, the hole injection layer may be doped with a p-type dopant. The electron-blocking layer may be placed between the hole transport layer (or hole injection layer) and the light-emitting layer, and can prevent light leakage by blocking the overflow of electrons from the light-emitting layer and confining excitons within the light-emitting layer. The hole transport layer or the electron-blocking layer may be a multi-layer, wherein a plurality of compounds may be used in each layer.

An electron buffer layer, a hole-blocking layer, an electron transport layer, an electron injection layer, or a combination thereof may be used between the light-emitting layer and the cathode. The electron buffer layer may be a multi-layer in order to control the injection of the electron and improve the interfacial properties between the light-emitting layer and the electron injection layer, wherein each layer may use two compounds simultaneously. The hole-blocking layer is placed between the electron transport layer (or electron injection layer) and the light-emitting layer, and is a layer that blocks holes from reaching the cathode, thereby improving the probability of recombination of electrons and holes in the light-emitting layer. The hole-blocking layer or electron transport layer may also be multiple layers, wherein a plurality of compounds may be used in each layer. In addition, the electron injection layer may be doped with an n-type dopant.

The light-emitting auxiliary layer, the hole auxiliary layer, or the electron-blocking layer may have an effect of improving the efficiency and/or lifespan of the organic electroluminescent device.

The light-emitting auxiliary layer may be a layer placed between an anode and a light-emitting layer, or between a cathode and a light-emitting layer. When the light-emitting auxiliary layer is placed between the anode and the light-emitting layer, the light-emitting auxiliary layer may be used for promoting the injection and/or transport of hole, or for preventing the overflow of electrons. When the light-emitting auxiliary layer is placed between the cathode and the light-emitting layer, the light-emitting auxiliary layer may be used for promoting the injection and/or transport of electron, or for preventing the overflow of holes.

In addition, the hole auxiliary layer may be placed between the hole transport layer (or hole injection layer) and the light-emitting layer, and may exhibit an effect of promoting or blocking the hole transport rate (or hole injection rate), thereby enabling the charge balance to be controlled. When an organic electroluminescent device includes two or more hole transport layers, the hole transport layer, which is further included, may be used as a hole auxiliary layer or an electron-blocking layer.

In the organic electroluminescent device of the present disclosure, it is preferable to place at least one layer selected from a chalcogenide layer, a metal halide layer, and a metal oxide layer (hereinafter referred to as a “surface layer”) on at least one inner surface of a pair of electrodes. Specifically, a chalcogenide (comprising oxide) layer of silicon and aluminum is preferably placed on an anode surface of an electroluminescent medium layer side, and a metal halide layer or a metal oxide layer is preferably placed on a cathode surface of an electroluminescent medium layer side. Such a surface layer provides operation stability for the organic electroluminescent device. For example, the chalcogenide includes SiO_{X} (1≤X≤2), AlO_{X} (1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

In addition, in an organic electroluminescent device of the present disclosure, a mixed region of an electron transport compound and a reductive dopant, or a mixed region of a hole transport compound and an oxidative dopant, may be placed on at least one surface of a pair of electrodes. In this way, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferred oxidative dopants include various Lewis acids and acceptor compounds, and preferred reductive dopants include alkali metals, alkali metal compounds, alkaline earth metals, rare earth metals, and mixtures thereof. In addition, an organic electroluminescent device having at least two light-emitting layers and emitting white light may be produced by using the reductive dopant layer as a charge-generating layer.

The manufacturing method of the organic electroluminescent device of the present disclosure is not limited, and the manufacturing method of the Device Example described below is only an example and is not limited thereto. A person skilled in the art can reasonably conceive the manufacturing method of the Device Examples as described below based on prior technologies. For example, there is no particular limitation on the mixing ratio of the first compound and the second compound, and thus a person skilled in the art can reasonably select it within a certain range based on prior technologies. For example, based on the total weight of the light-emitting layer material, the total weight of the first compound and the second compound accounts for 80.0% to 99.5% of the total weight of the light-emitting layer, the weight ratio of the first compound and the second compound may be between 1:99 and 99:1, the weight ratio of the first compound and the second compound may be between 20:80 and 99:1, or the weight ratio of the first compound and the second compound may be between 50:50 and 90:10. In the manufacture of devices, when forming a light-emitting layer by co-depositing two or more host materials and a light-emitting material, the two or more host materials and the light-emitting material may each be placed in different evaporation sources and co-deposited to form a light-emitting layer, or a pre-mixed mixture of two or more host materials may be placed in the same evaporation source and then co-deposited with a light-emitting material placed on another evaporation source to form a light-emitting layer. This pre-mixing method can further save evaporation sources. According to one embodiment, the first compound, the second compound, and the light-emitting material of the present disclosure may each be placed in different evaporation sources and co-deposited to form a light-emitting layer, or a pre-mixed mixture of the first compound and the second compound may be placed in the same evaporation source and then co-deposited with a light-emitting material placed in another evaporation source to form a light-emitting layer. The light-emitting layer of the organic electroluminescent device according to one embodiment may be a single layer in which light is emitted, or may be a plurality of layers in which two or more layers are laminated. The light-emitting layer may further include one or more dopants, and the doping concentration of the dopant compound with respect to the host compound of the light-emitting layer may be less than 20 wt %, and preferably less than 10 wt %.

The organic electroluminescent device according to one embodiment of the present disclosure may be an organic electroluminescent device having a tandem structure. In the case of a tandem organic electroluminescent device according to one embodiment, a single light-emitting unit(light-emitting unit) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer that is stacked between the first and second electrodes and emits light in a specific wavelength range. The organic electroluminescent device may include a plurality of light-emitting units, and each of the light-emitting units may include a hole transport band, a light-emitting layer, and an electron transport band, and the hole transport band may include a hole injection layer and a hole transport layer, and the electron transport band may include an electron transport layer and an electron injection layer. According to one embodiment, three or more light-emitting layers may be included in the light-emitting unit. A plurality of light-emitting units may emit the same color or different colors. Additionally, one light-emitting unit may include one or more light-emitting layers, and the plurality of light-emitting layers may be light-emitting layers of the same or different colors. This may include one or more charge generation layers located between each light-emitting unit. The charge generation layer refers to the layer in which holes and electrons are generated when voltage is applied. When there are three or more light-emitting units, a charge generation layer may be located between each light-emitting unit. Here, the plurality of charge generation layers may be the same as or different from one another. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of an anode and a cathode without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

An organic electroluminescent device according to one embodiment may further comprise at least one dopant in the light-emitting layer.

The dopant included in the organic electroluminescent device of the present disclosure may be at least one phosphorescent or fluorescent dopant, preferably a fluorescent dopant. For example, the phosphorescent dopant material applied to the organic electroluminescent device of the present disclosure is not particularly limited, but may be a complex compound(s) of a metal atom(s) selected from iridium (Ir), osmium (Os), copper (Cu), and platinum (Pt), preferably an ortho-metallated complex compound(s) of a metal atom(s) selected from iridium (Ir), osmium (Os), copper (Cu), and platinum (Pt), and more preferably an ortho-metallated iridium complex compound(s).

The dopant included in the organic electroluminescent device of the present disclosure may use the compound represented by the following Formula D, but is not limited thereto.

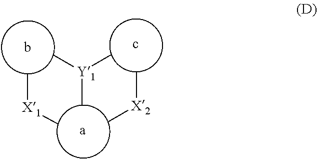

In Formula D,
Y′₁ represents B or N;
X′₁ and X′₂ each independently represent O, S, NR′, or BR′;
ring a, ring b, and ring c each independently represent a substituted or unsubstituted aromatic hydrocarbon ring having 5 to 50 carbon atoms, a substituted or unsubstituted aromatic hetero ring having 2 to 50 carbon atoms, a substituted or unsubstituted aliphatic ring having 3 to 30 carbon atoms, or a substituted or unsubstituted aliphatic aromatic mixed ring having 3 to 30 carbon atoms;
each R′ independently represents hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or L′₄-N-(Ar′₄)(Ar′₅); or may be linked to the adjacent substituents to form a ring(s);
each L′₄ independently represents a single bond, a substituted or unsubstituted (C₆-C₃₀)arylene, or a substituted or unsubstituted (3- to 30-membered)heteroarylene; and
Ar′₄ and Ar′₅ each independently represent hydrogen, a substituted or unsubstituted (C₁-C₃₀)alkyl, a substituted or unsubstituted (C₂-C₃₀)alkenyl, a substituted or unsubstituted fused ring group of a (C₃-C₃₀)aliphatic ring(s) and a (C₆-C₃₀)aromatic ring(s), a substituted or unsubstituted (C₆-C₃₀)aryl, or a substituted or unsubstituted (3- to 30-membered)heteroaryl.

According to one embodiment of the present disclosure, Formula D may be represented by the following Formula D-1.

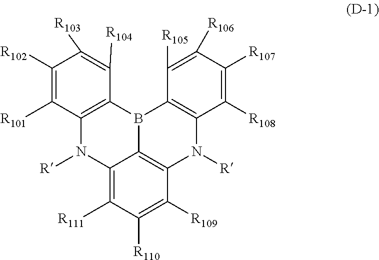

In Formula D-1,
R₁₀₁ to R₁₁₁ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N-(Ar′₄)(Ar′₅); or may be linked to the adjacent substituents to form a ring(s);
each R′ independently represents hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or L′₄-N-(Ar′₄)(Ar′₅); or may be linked to at least one of R₁₁₀₁, R₁₀₈, R₁₀₉, and R₁₁₁ to form a ring(s);
each L′₄ independently represents a single bond, a substituted or unsubstituted (C₆-C₃₀)arylene, or a substituted or unsubstituted (3- to 30-membered)heteroarylene; and
Ar′₄ and Ar′₅ each independently represent hydrogen, a substituted or unsubstituted (C₁-C₃₀)alkyl, a substituted or unsubstituted (C₂-C₃₀)alkenyl, a substituted or unsubstituted fused ring group of a (C₃-C₃₀)aliphatic ring(s) and a (C₆-C₃₀)aromatic ring(s), a substituted or unsubstituted (C₆-C₃₀)aryl, or a substituted or unsubstituted (3- to 30-membered)heteroaryl.

Preferably, R₁₁₀₁ to R₁₁₁ each independently represent hydrogen, deuterium, a substituted or unsubstituted (C₁-C₂₀)alkyl, a substituted or unsubstituted (C₆-C₂₅)aryl, a substituted or unsubstituted (5- to 20-membered)heteroaryl, or -L′₄-N-(Ar′₄)(Ar′₅); or may be linked to the adjacent substituents to form a ring(s).

More preferably, R₁₁ to R₁₁₁ each independently represent hydrogen; deuterium; an unsubstituted (C₁-C₁₀)alkyl; a (C₆-C₁₈)aryl unsubstituted or substituted with at least one of (C₁-C₁₀)alkyl, (13- to 18-membered)heteroaryl, and di(C₆-C₁₈)arylamino; a (5- to 18-membered)heteroaryl unsubstituted or substituted with at least one (C₁-C₁₀)alkyl; or -L′₄-N-(Ar′₄)(Ar′₅); or may be linked to the adjacent substituents to form a ring(s). For example, R₁₁ to R₁₁₁ may each independently be hydrogen, methyl, tert-butyl, a substituted or unsubstituted phenyl, a biphenyl, a terphenyl, a triphenylenyl, a carbazolyl, a phenoxazinyl, a phenothiazinyl, a dimethylacridinyl, a dimethylxanthenyl, a diphenylamino unsubstituted or substituted with at least one of methyl and diphenylamino, a phenylnaphthylamino, a dibiphenylamino, a phenylamino substituted with phenylcarbazolyl or dibenzofuranyl, or a (17- to 21-membered)heteroaryl substituted with at least one of methyl and phenyl; or may be linked to the adjacent substituents to form a benzene ring, an indole ring substituted with at least one of phenyl and diphenylamino, a benzofuran ring, a benzothiophene ring, or a 19-membered hetero ring substituted with at least one methyl. The substituted phenyl may be substituted at least one of methyl, carbazolyl, dibenzofuranyl, diphenylamino, phenoxazinyl, phenothiazinyl, and dimethylacridinyl.

According to one embodiment of the present disclosure, Formula D may be represented by the following Formula D-2 or D-3.

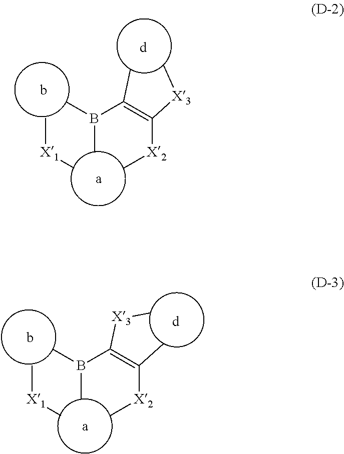

In Formulas D-2 and D-3,
X′₁ and X′₂ each independently represent 0, S, NR′, or BR′;
ring a, ring b, and ring d each independently represent a substituted or unsubstituted aromatic hydrocarbon ring having 5 to 50 carbon atoms, a substituted or unsubstituted aromatic hetero ring having 2 to 50 carbon atoms, a substituted or unsubstituted aliphatic ring having 3 to 30 carbon atoms, or a substituted or unsubstituted aliphatic aromatic mixed ring having 3 to 30 carbon atoms; and
R′, L′₄, Ar′₄, and Ar′₅ are as defined in Formula D.

According to one embodiment of the present disclosure, the specific examples of Formula D are as follows, but are not limited thereto.

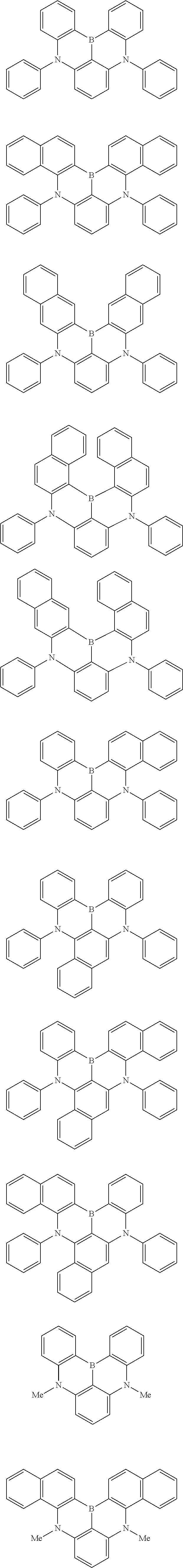

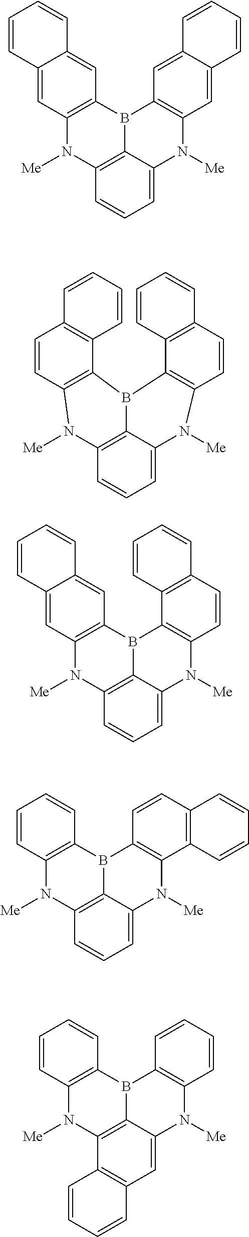

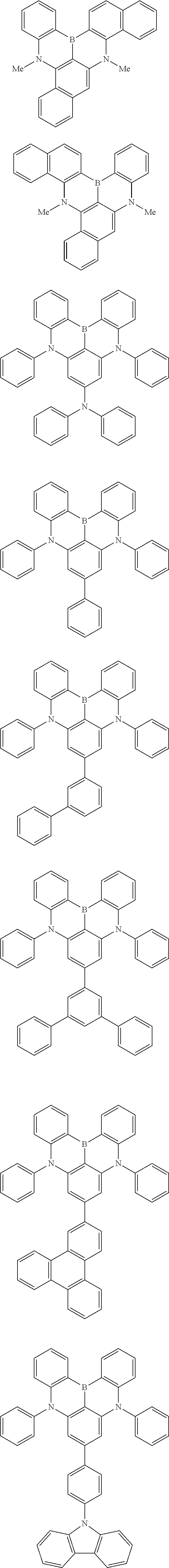

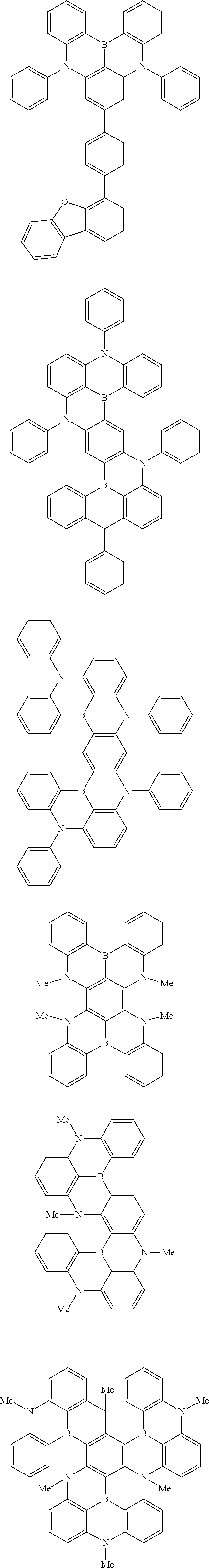

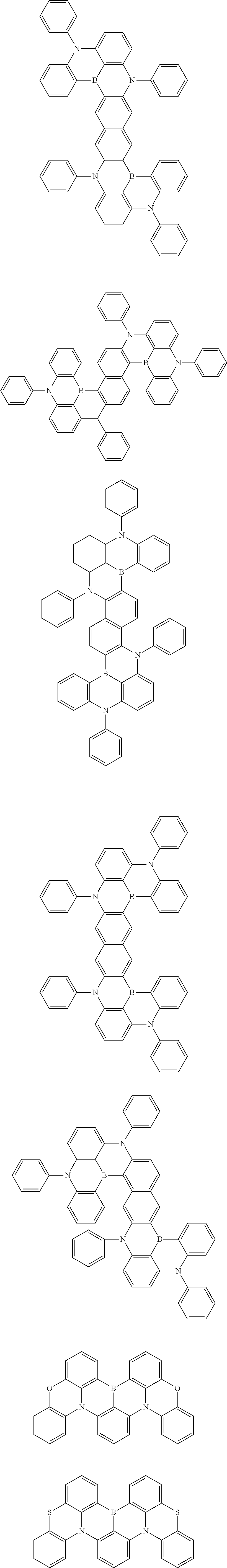

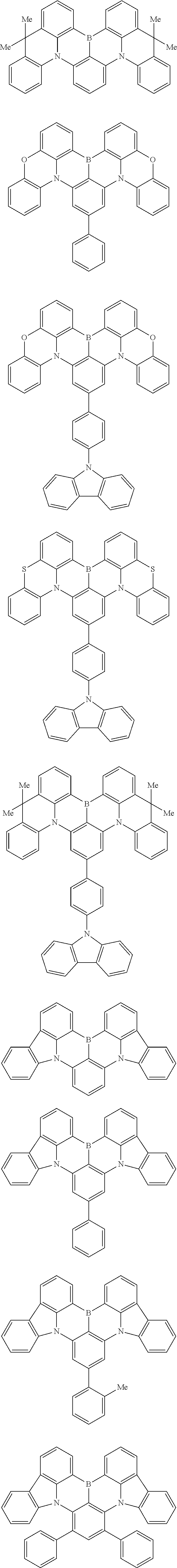

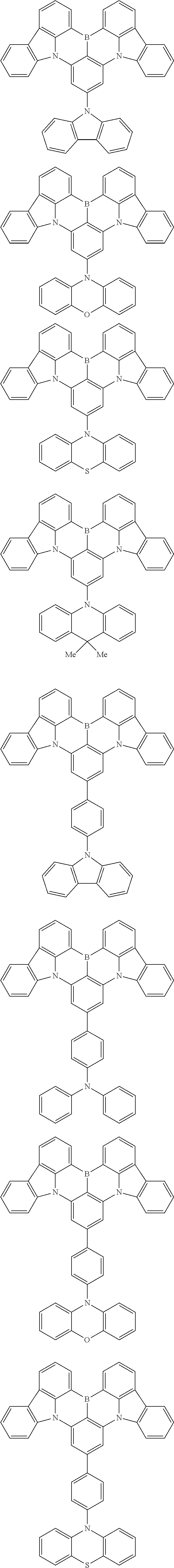

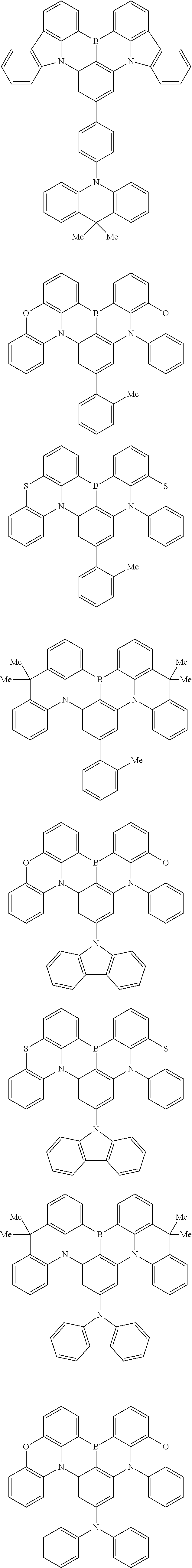

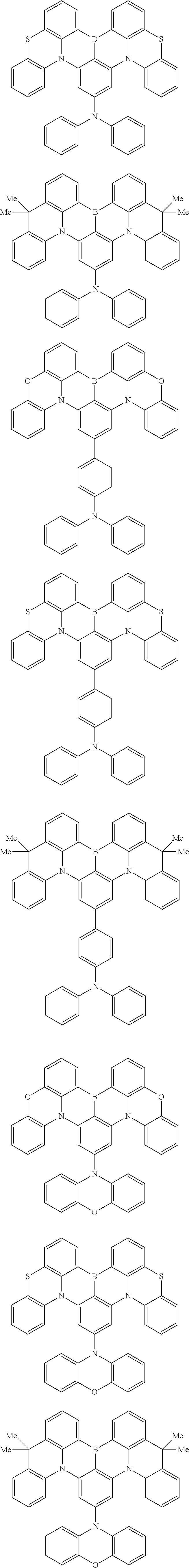

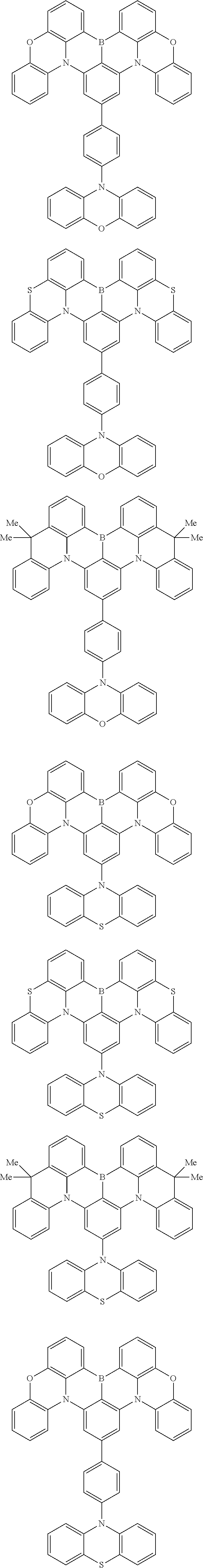

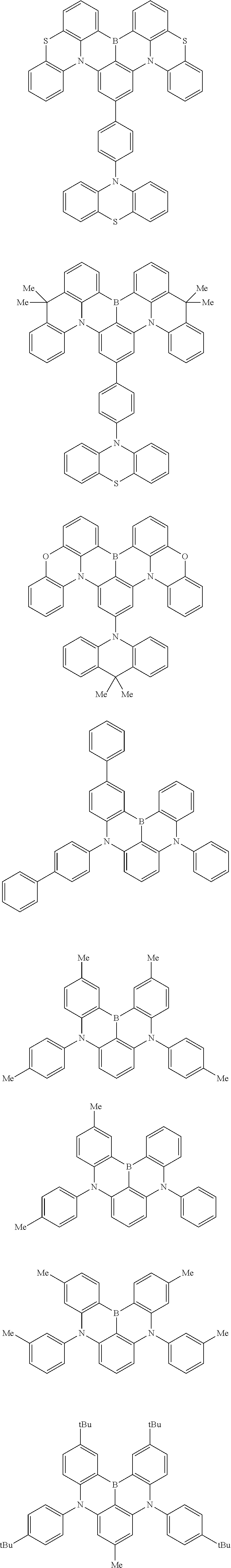

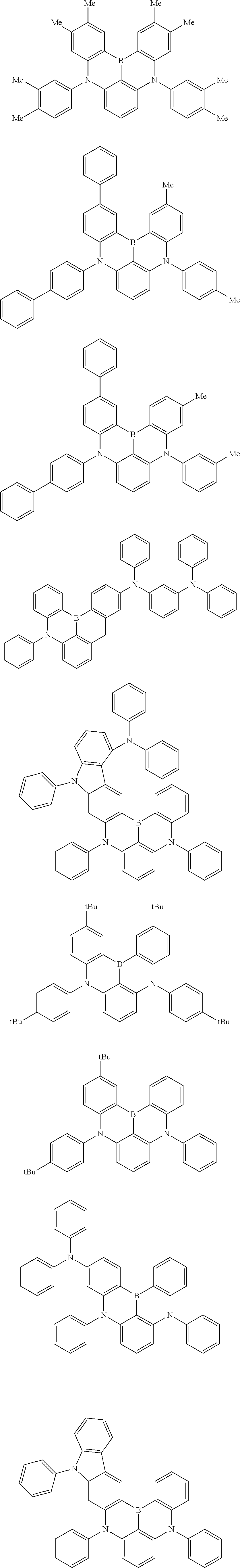

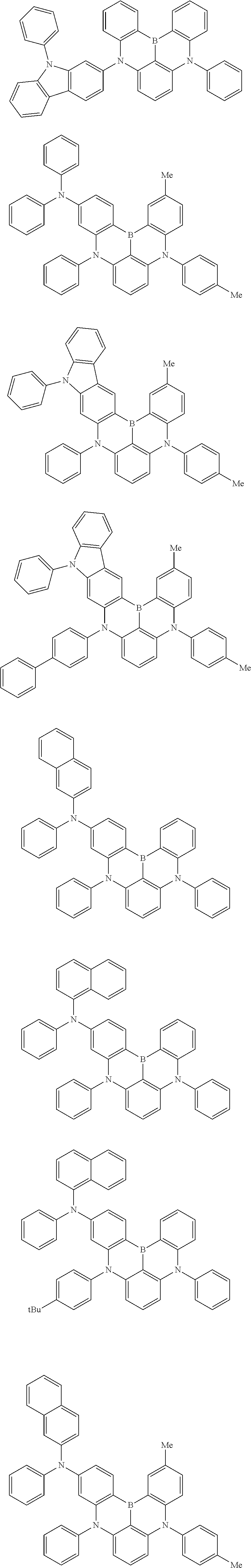

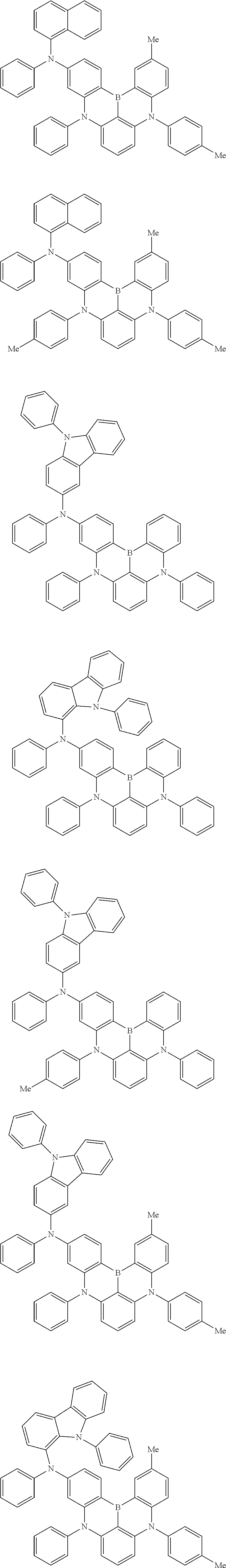

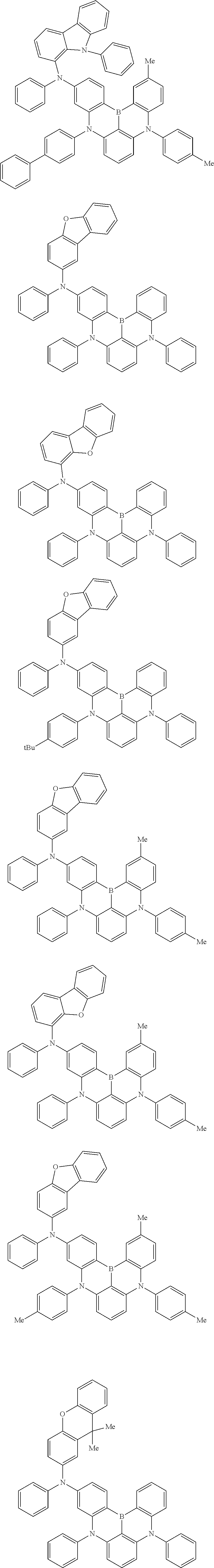

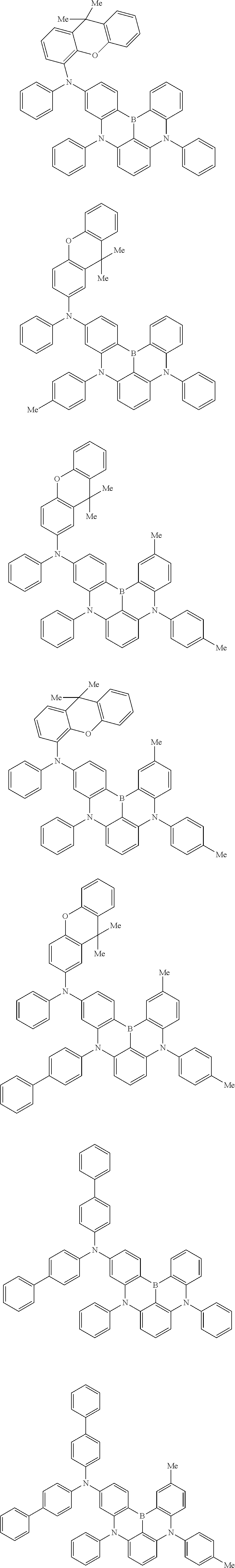

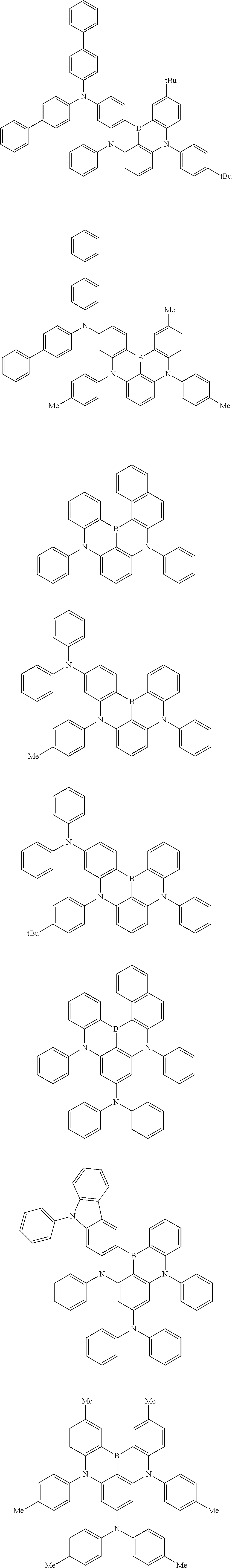

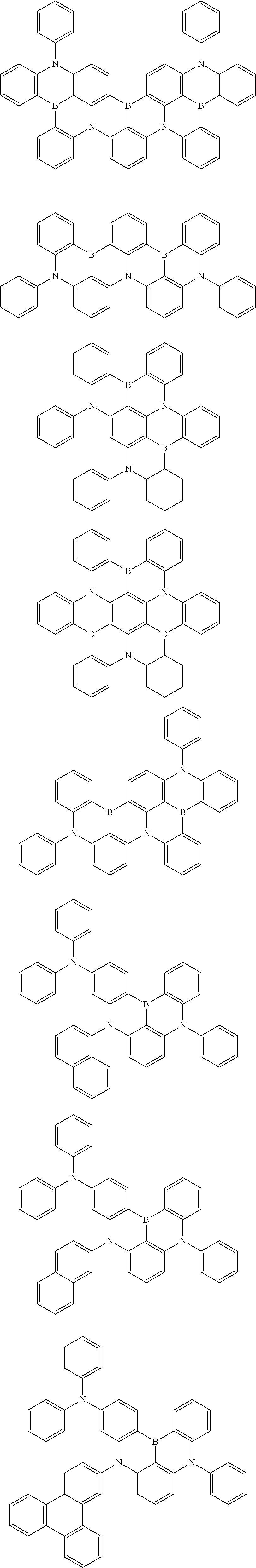

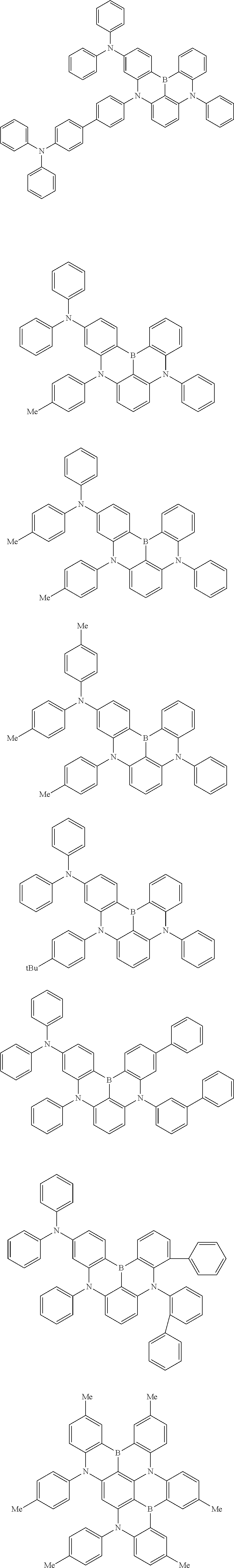

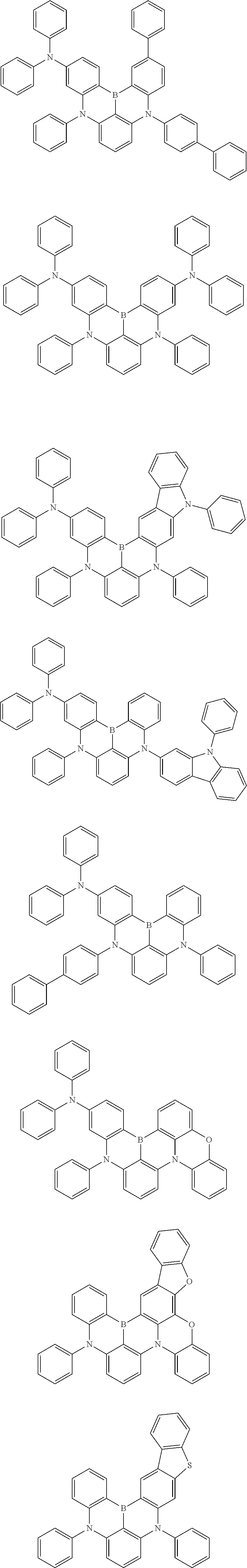

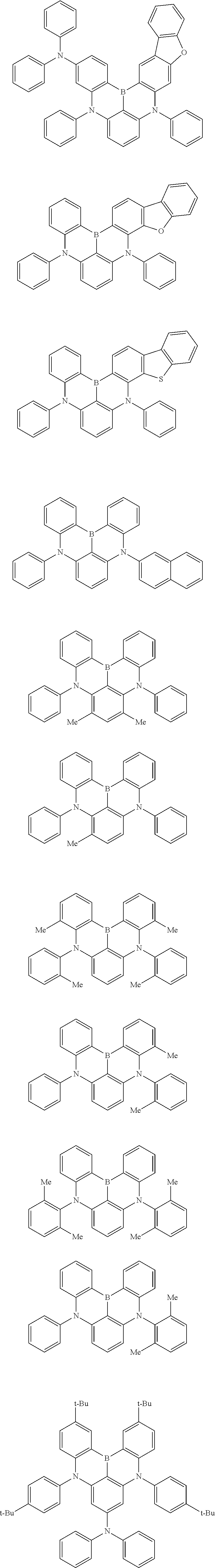

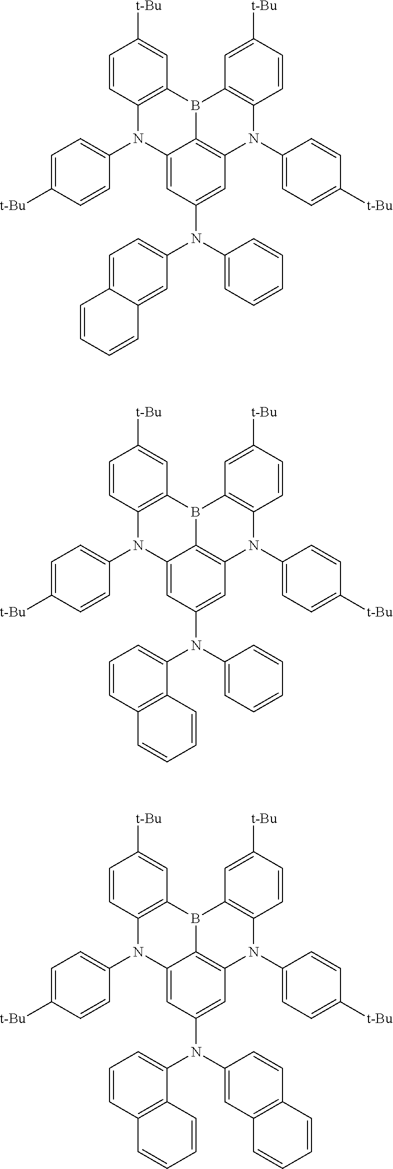 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccc6ccccc6c5)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5cccc6ccccc56)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5cccc6ccccc56)cc2c43)cc1]

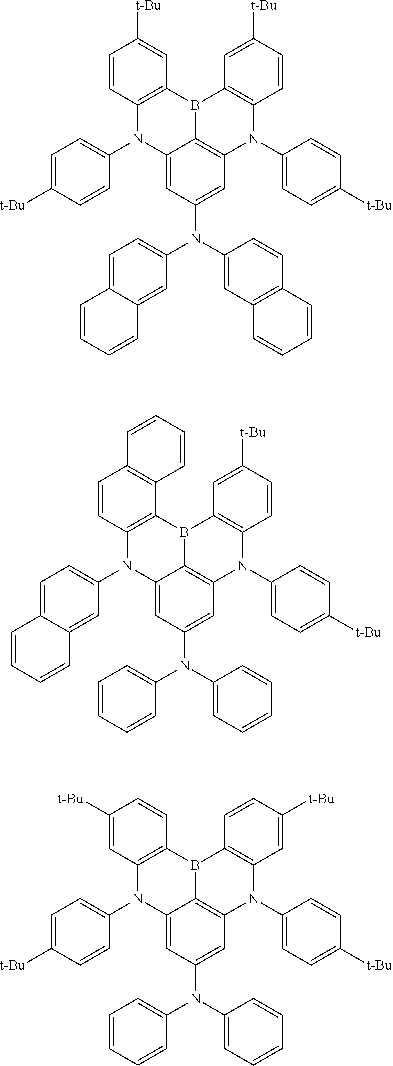 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccc6ccccc6c5)c5ccc6ccccc6c5)cc2c43)cc1 ; CC(C)(C)c1ccc(N2c3ccc(C(C)(C)C)cc3B3c4c2cc(N(c2ccccc2)c2ccccc2)cc4N(c2ccc4ccccc4c2)c2ccc4ccccc4c23)cc1 ; CC(C)(C)c1ccc(N2c3cc(C(C)(C)C)ccc3B3c4ccc(C(C)(C)C)cc4N(c4ccc(C(C)(C)C)cc4)c4cc(N(c5ccccc5)c5ccccc5)cc2c43)cc1]

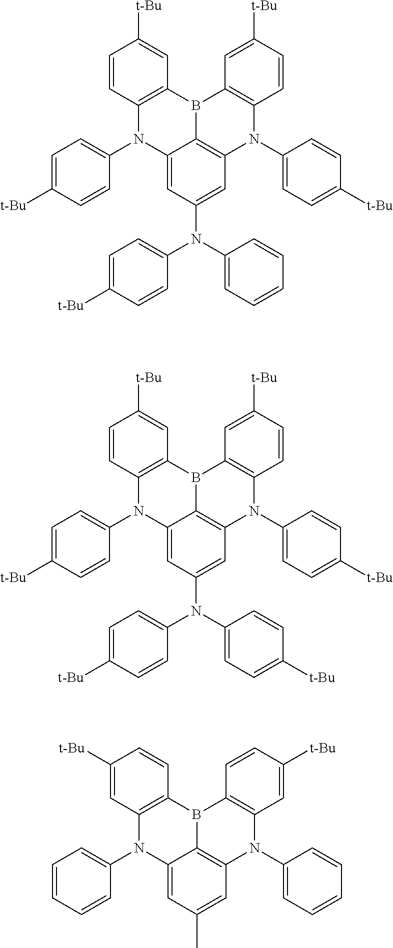 [SMILES: CC(C)(C)c1ccc(N(c2ccccc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; CC(C)(C)c1ccc(N(c2ccc(C(C)(C)C)cc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; Cc1cc2c3c(c1)N(c1ccccc1)c1cc(C(C)(C)C)ccc1B3c1ccc(C(C)(C)C)cc1N2c1ccccc1]

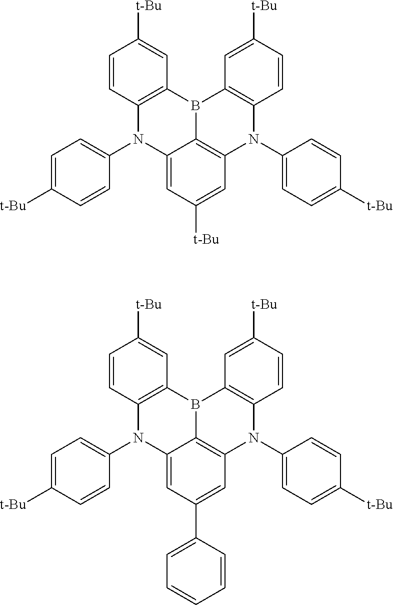 [SMILES: CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1]

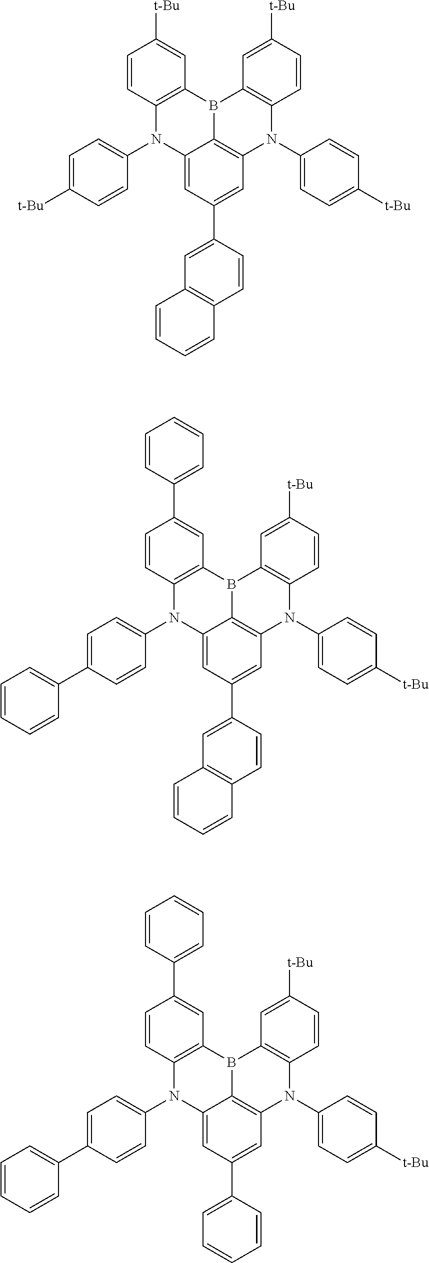 [SMILES: CC(C)(C)c1ccc(N2c3ccc(C(C)(C)C)cc3B3c4cc(C(C)(C)C)ccc4N(c4ccc(C(C)(C)C)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccccc5)cc2c43)cc1]

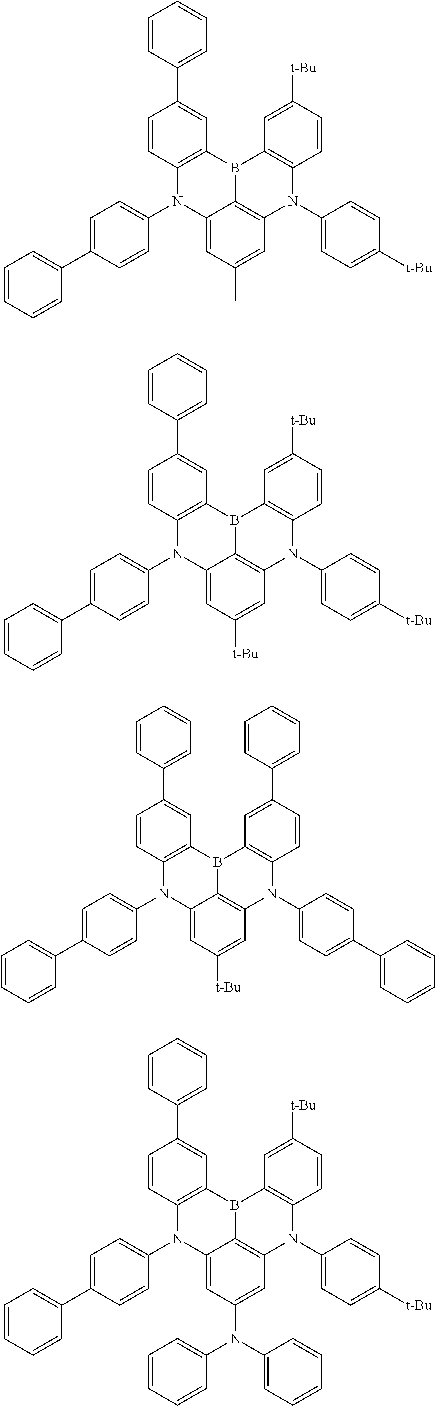 [SMILES: Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(N(c5ccccc5)c5ccccc5)cc2c43)cc1]

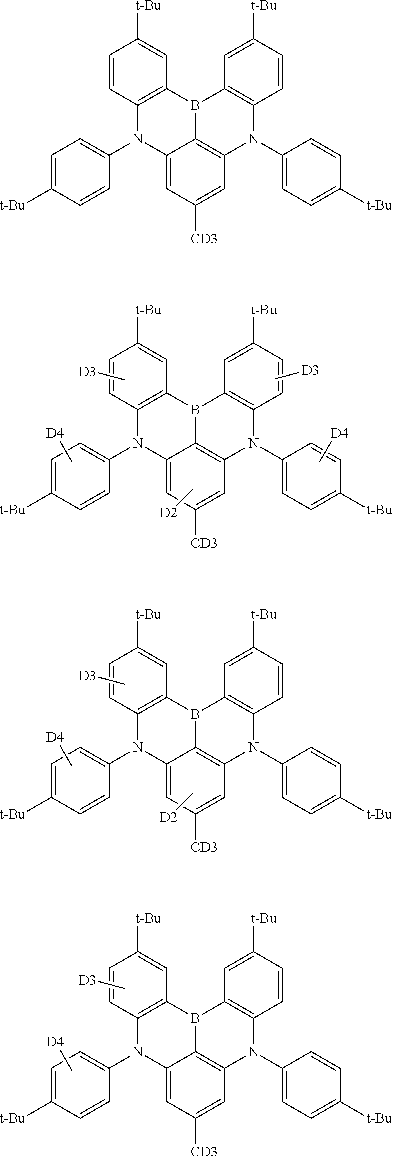

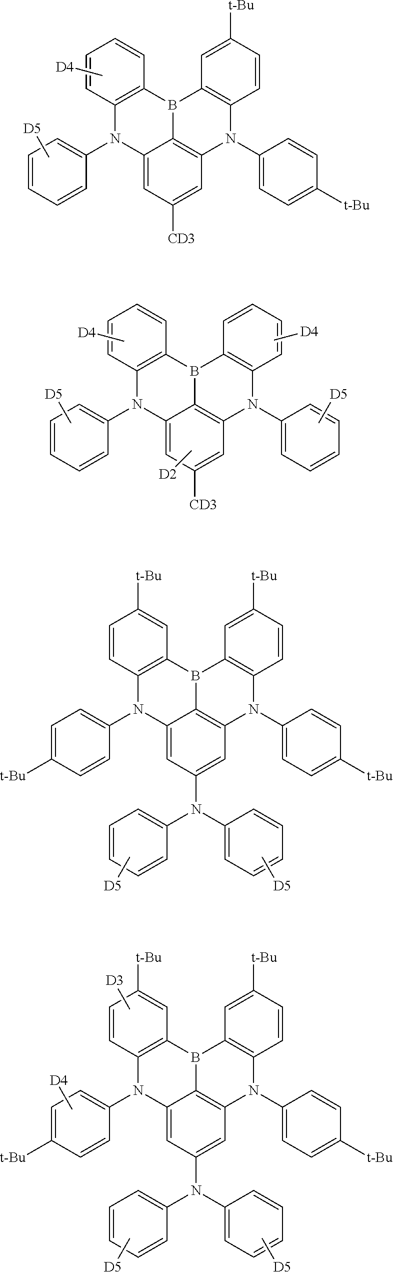

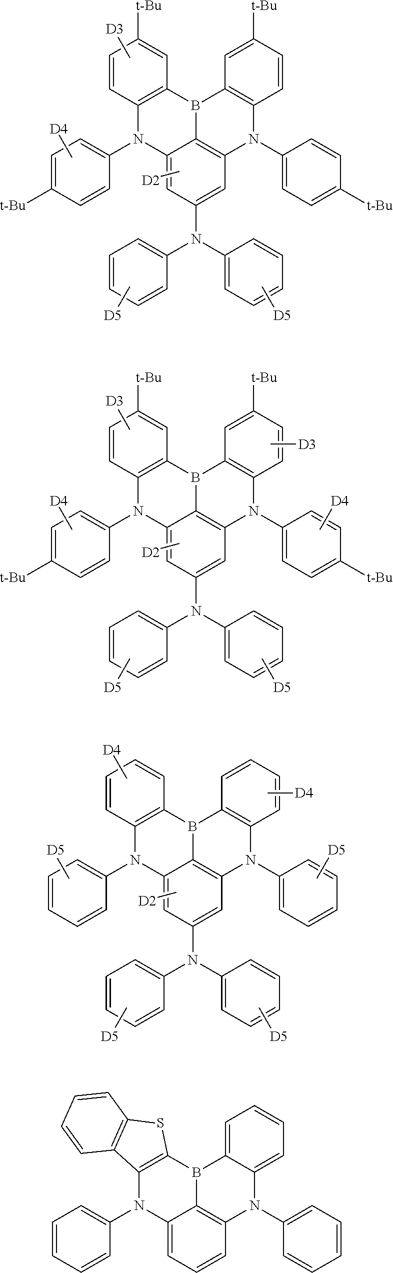

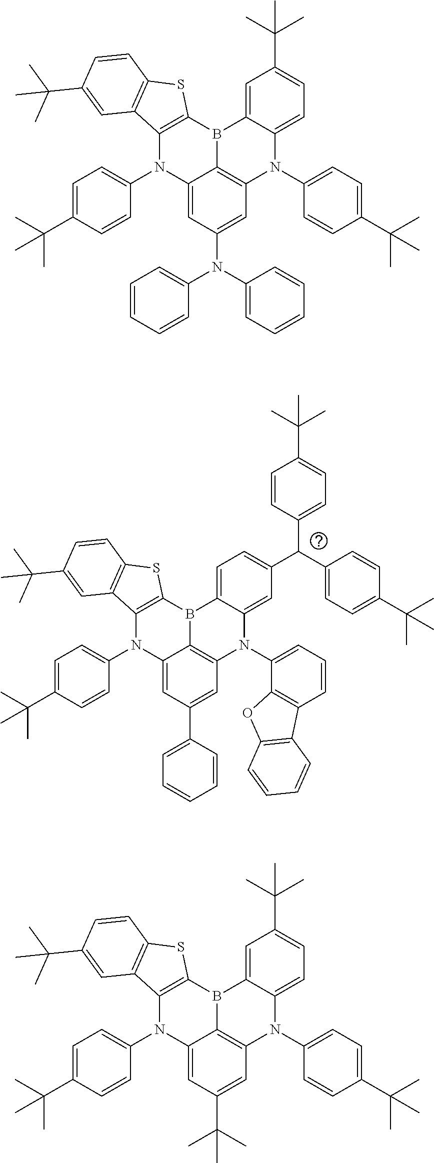

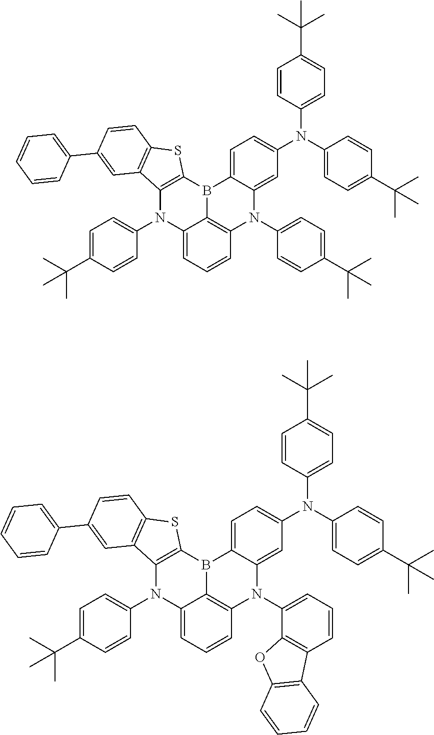 [SMILES: CC(C)(C)c1ccc(N(c2ccc(C(C)(C)C)cc2)c2ccc3c(c2)N(c2ccc(C(C)(C)C)cc2)c2cccc4c2B3c2sc3ccc(-c5ccccc5)cc3c2N4c2ccc(C(C)(C)C)cc2)cc1 ; CC(C)(C)c1ccc(N(c2ccc(C(C)(C)C)cc2)c2ccc3c(c2)N(c2cccc4c2oc2ccccc24)c2cccc4c2B3c2sc3ccc(-c5ccccc5)cc3c2N4c2ccc(C(C)(C)C)cc2)cc1]

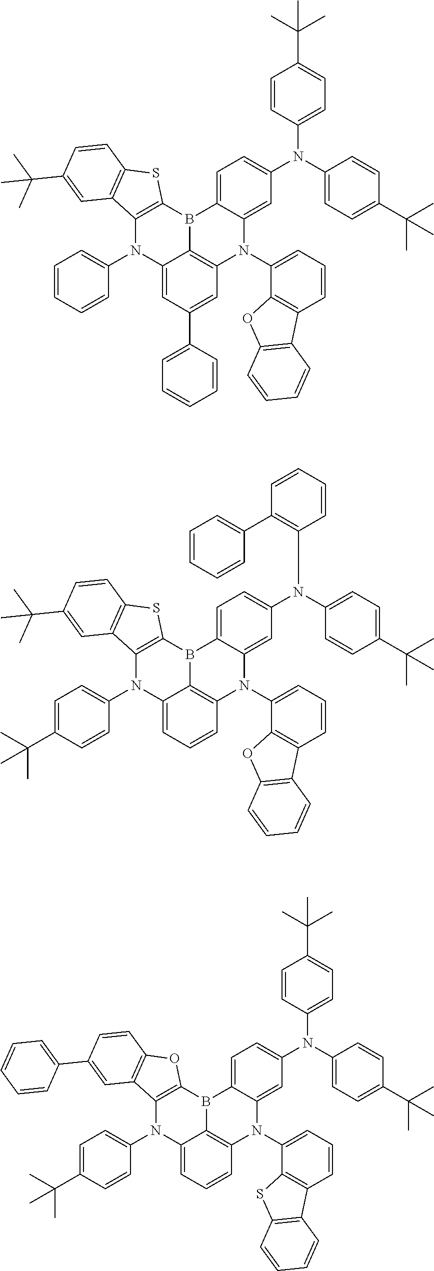

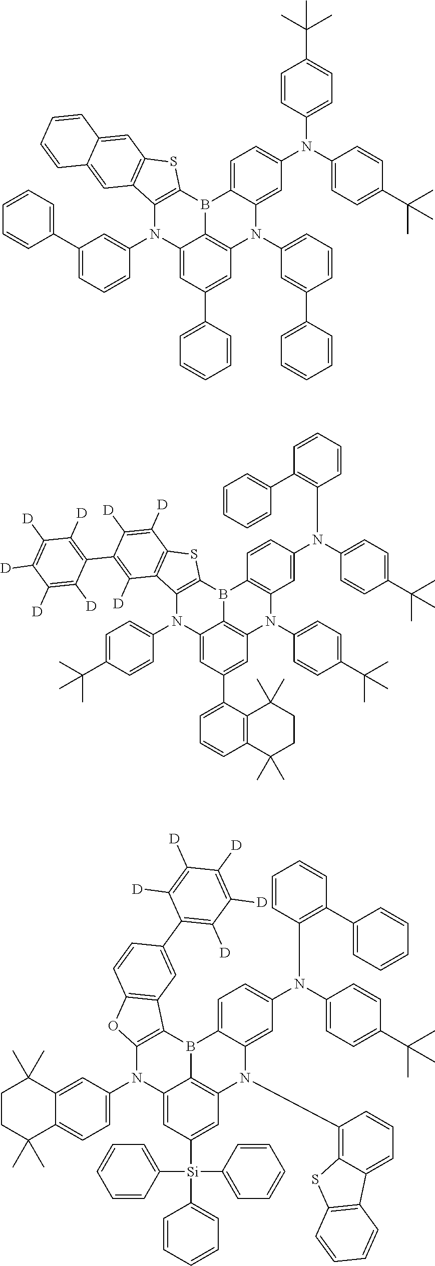

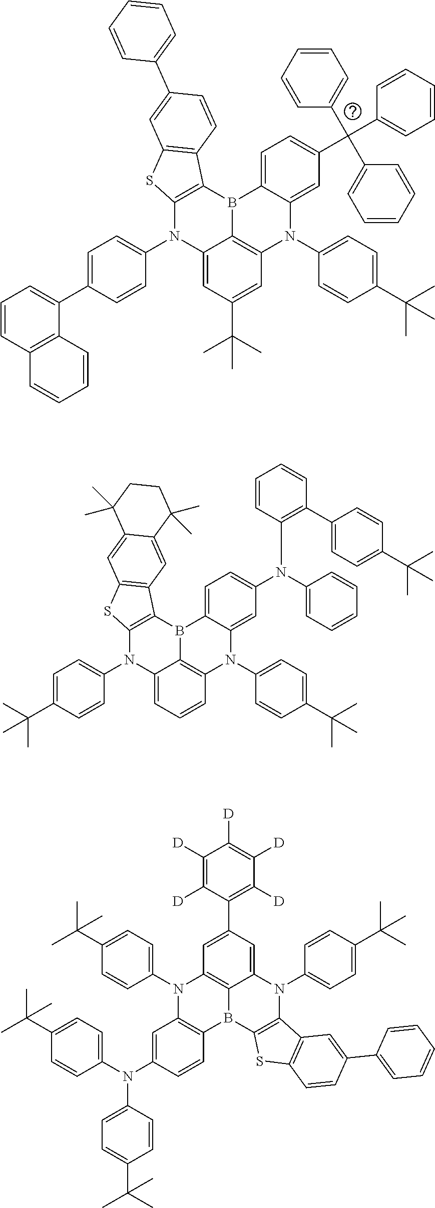

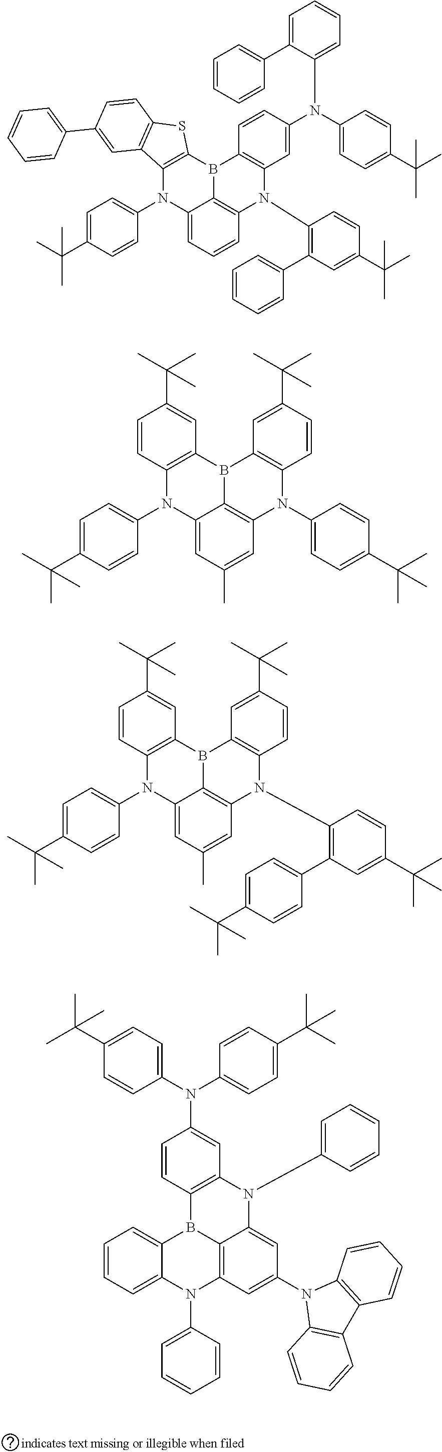

In the above compounds, D2 to D5 each mean that 2 to 5 hydrogens are replaced with deuterium.

The formation of each layer of the organic electroluminescent device of the present disclosure can be accomplished by applying any one of dry film-forming methods such as vacuum deposition, sputtering, plasma, ion plating methods, etc., or wet film-forming methods such as spin coating, dip coating, flow coating methods, etc. When using a wet film-forming method, a thin film can be formed by dissolving or dispersing materials forming each layer into any suitable solvent such as ethanol, chloroform, tetra hydrofuran, dioxane, etc. The solvent can be any one in which the materials forming each layer can be dissolved or dispersed, provided that there is no problem in film formability.

According to one embodiment of the present disclosure, when forming a layer of the first host compound and the second host compound of the present disclosure, the layer can be formed by the methods listed above, and often can be formed by a co-deposition or mixed deposition process. The co-deposition is a method for mixing two or more materials into each individual crucible source and applying current to both cells simultaneously to evaporate the materials. The mixed deposition is a method for mixing two or more materials in one crucible source before deposition and then applying current to one cell to evaporate the materials.

According to one embodiment of the present disclosure, when the first host compound and the second host compound are present in the same layer or different layers in the organic electroluminescent device, the two host compounds may be individually formed into films. For example, the second host material may be deposited after depositing the first host material.

The plurality of host materials according to one embodiment of the present disclosure may be used as light-emitting materials for a white organic light-emitting device. The white organic light-emitting device has various suggested structures such as a side-by-side arrangement method, a stacking arrangement method, or a CCM (color conversion material) method, etc. depending on the arrangement of R (red), G (green), YG (yellowish green), or B (blue) light-emitting units. In addition, the plurality of host materials according to another embodiment of the present disclosure may also be applied to the organic electroluminescent device comprising a QD (quantum dot). In addition, the plurality of host materials according to the present disclosure can be used for the manufacture of display devices such as smartphones, tablets, notebooks, PCs, TVs, or display devices for vehicles, or lighting devices such as outdoor or indoor lighting.

Hereinafter, the preparation methods of the compounds according to the present disclosure and the properties thereof, and the light-emitting properties of an organic electroluminescent device (OLED) comprising an organic electroluminescent compound or a plurality of host materials according to the present disclosure will be explained in detail with reference to the representative compounds of the present disclosure. However, the following examples only describe the properties of OLED comprising the organic electroluminescent compound or the plurality of host materials according to the present disclosure for a detailed understanding of the present disclosure, and the present disclosure is not limited to the following examples.

### Example 1: Synthesis of Compound H1-206-D17

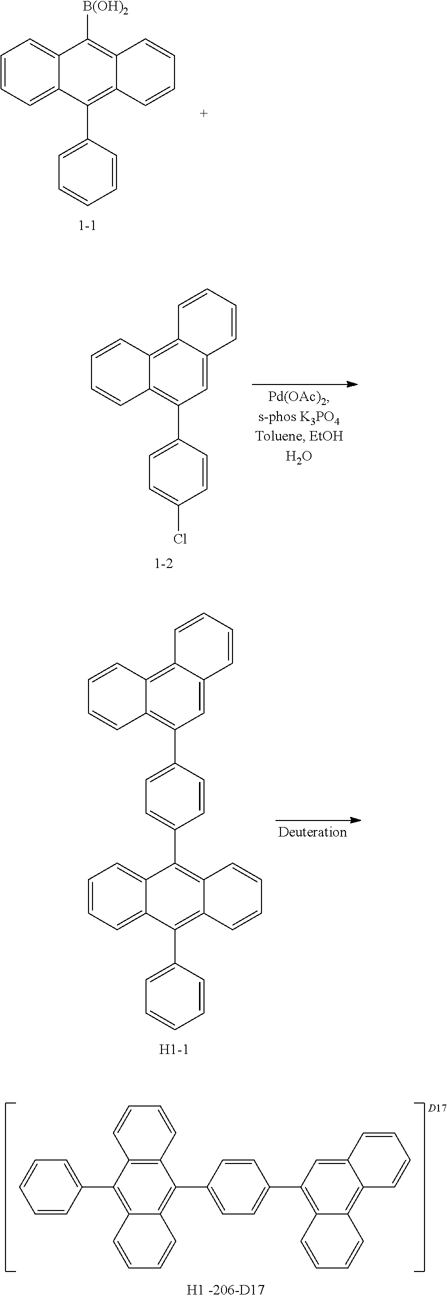

### 1) Synthesis of Compound H1-1

In a flask, Compound 1-1 (24.8 g, 86 mmol), Compound 1-2 (33.3 g, 112 mmol), Pd(OAc)₂ (1.35 g, 6 mmol), SPhos (5.3 g, 13 mmol), K₃PO₄ (55 g, 258 mmol), toluene (500 mL), ethanol (125 mL), and distilled water (125 mL) were added, and the mixture was stirred under reflux at 120° C. for 3 hours. After the reaction was completed, the mixture was cooled to room temperature, and an organic layer was extracted with ethyl acetate (EA). The residual moisture was removed by drying over magnesium sulfate, and the organic layer was separated by column chromatography to obtain Compound H1-1 (20.2 g, yield: 46%).

| | MW | M.P. |
|---|---|---|
| H1-1 | 506.65 | 293° C. |

### 2) Synthesis of Compound H1-206-D17

Compound H1-1 was synthesized by selecting from among the deuteration methods disclosed in Korean Patent Nos. 10⁻²²⁸³⁸⁴⁹ and 10⁻¹⁴²⁷⁴⁵⁷, etc. to obtain Compound H1-206-D17 (13 g, yield: 99%, MS: [M+H]⁺=523.2).

### Example 2: Synthesis of Compound H3-1

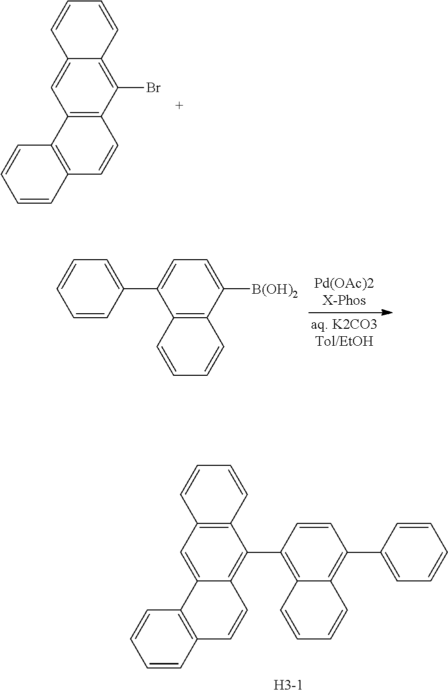

7-Bromotetraphene (15 g, 48.88 mmol), (4-phenylnaphthalen-1-yl)boronic acid (14.53 g, 58.59 mmol), Pd(OAc)₂ (0.54 g, 2.44 mmol), XPhos (2.79 g, 5.85 mmol), K₂CO₃ (20.24 g, 146.48 mmol), toluene (400 mL), distilled water (100 mL), and ethanol (70 mL) were mixed, and the mixture was stirred under reflux at 120° C. After 3 hours, the mixture was cooled to room temperature, distilled water was added thereto, and the organic layer was then extracted with EA. The organic layer was dried over magnesium sulfate and filtered under reduced pressure. The organic layer was distilled under reduced pressure and separated by column chromatography to obtain Compound H3-1 (17.4 g, yield: 82.67%).

| | MW | M.P. |
|---|---|---|
| H3-1 | 430.55 | 225.6° C. |

### Example 3: Synthesis of Compound H3-93-D18

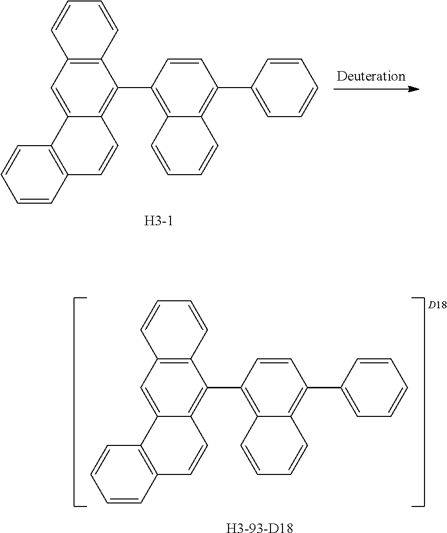

Compound H3-1 (38.5 g) was synthesized by selecting from among the deuteration methods disclosed in Korean Patent Nos. 10⁻²²⁸³⁸⁴⁹ and 10⁻¹⁴²⁷⁴⁵⁷, etc. to obtain Compound H3-93-D18 (27.0 g yield: 67.52% MS: [M+H]⁺=448.1).

| | MW | M.P. |
|---|---|---|
| H3-93-D18 | 447.1 | 230.1° C. |

### Example 4: Synthesis of Compound H3-4

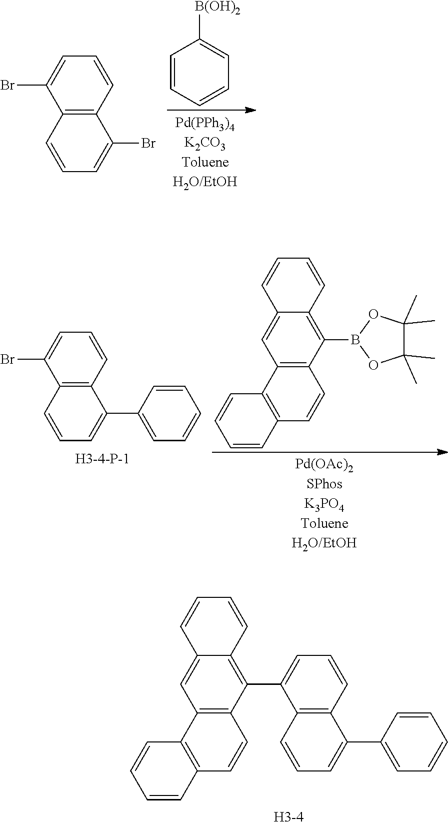

### 1) Synthesis of Compound H3-4-P-1

1,5-Dibromonaphthalene (60 g, 209.8 mmol), phenyl boronic acid (28.1 g, 230.8 mmol), Pd(PPh₃)₄ (12.1 g, 10.5 mmol), K₂CO₃ (72.5 g, 524.5 mmol), toluene (1 L), distilled water (250 mL), and ethanol (250 mL) were mixed, and the mixture was stirred under reflux at 120° C. After 3 hours, the mixture was cooled to room temperature, distilled water was added thereto, and the organic layer was then extracted with methylene chloride (MC). The organic layer was dried over magnesium sulfate and filtered under reduced pressure. The organic layer was distilled under reduced pressure, and then separated by column chromatography using silica to obtain Compound H3-4-P-1 (40.8 g, yield: 68.7%).

### 2) Synthesis of Compound H3-4

Compound H3-4-P-1 (40.8 g, 144.1 mmol), 4,4,5,5-tetramethyl-2-(tetraphen-7-yl)-1,3,2-dioxaborolane (56.1 g, 158.5 mmol), Pd(OAc)₂ (1.6 g, 7.2 mmol), SPhos (7.1 g, 17.3 mmol), K₃PO₄ (76.5 g, 360.2 mmol), toluene (900 mL), distilled water (120 mL), and ethanol (120 mL) were mixed, and the mixture was stirred under reflux at 120° C. After 4 hours, the mixture was cooled to room temperature, distilled water was added thereto, and the organic layer was then extracted with toluene. The organic layer was dried over magnesium sulfate and filtered under reduced pressure. The organic layer was distilled under reduced pressure, and then separated by column chromatography using silica to obtain Compound H3-4 (40.0 g, yield: 64.5%).

| | MW | M.P. |
|---|---|---|
| H3-4 | 430.55 | 271° C. |

### Example 5: Synthesis of Compound H3-94-D17

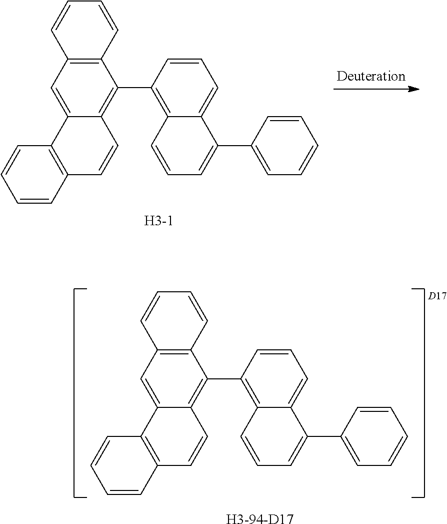

Compound H3-4 (40.0 g) was synthesized by selecting from among the deuteration methods disclosed in Korean Patent Nos. 10⁻²²⁸³⁸⁴⁹ and 10⁻¹⁴²⁷⁴⁵⁷, etc. to obtain Compound H3-94-D17 (27.5 g, yield: 66.3%, MS: [M+H]⁺=447.2).

| | MW | M.P. |
|---|---|---|
| H3-94-D17 | 446.65 | 271.9° C. |

Hereinafter, for a detailed understanding of the present disclosure, a preparation method of an organic electroluminescent device comprising a plurality of host materials, and/or organic electroluminescent compounds according to the present disclosure and its properties are described.

### Device Example 1: Producing an OLED by Depositing the Compound According to the Present Disclosure

An OLED according to the present disclosure was produced. A transparent electrode indium tin oxide (ITO) thin film on a glass substrate for an OLED (GEOMATEC CO., LTD.) was subjected to an ultrasonic washing with acetone and isopropyl alcohol, sequentially, and thereafter was stored in isopropyl alcohol and then used. The ITO substrate was then mounted on a substrate holder of a vacuum vapor deposition apparatus. Next, Compound HI-1 was introduced into a cell of the vacuum vapor deposition apparatus, and Compound HT-1 was introduced into another cell of the vacuum vapor deposition apparatus. The two materials were evaporated at different rates, and Compound HI-1 was deposited in a doping amount of 5 wt % based on the total amount of Compound HI-1 and Compound HT-1 to form a hole injection layer having a thickness of 10 nm. Compound HT-1 was then deposited on the hole injection layer to form a first hole transport layer having a thickness of 80 nm. Next, Compound HT-2 was introduced into another cell of the vacuum vapor deposition apparatus and was evaporated by applying an electric current to the cell to thereby form a second hole transport layer having a thickness of 15 nm on the first hole transport layer. After formation of the hole injection layer and the hole transport layers, a first light-emitting layer was formed thereon as follows. Compound Host 1 was introduced into a cell of the vacuum vapor deposition apparatus as a host, and Compound D-1 was introduced into another cell as a dopant. The two materials were evaporated at different rates, and the dopant was deposited in a doping amount of 2 wt % based on the total amount of the host and dopant to form a first light-emitting layer with a thickness of 5 nm on the second hole transport layer. A second light-emitting layer was then formed on the first light-emitting layer as follows. The two compounds shown in Table 1 below were introduced into a cell of the vacuum vapor deposition apparatus as hosts, and Compound D-1 was introduced into another cell as a dopant. Next, the two host materials were deposited at a weight ratio of 1:1, and the dopant was deposited in a doping amount of 2 wt % to form a second light-emitting layer with a thickness of 13 nm. After deposition of the light-emitting layers, Compound ET-1 was deposited as a hole-blocking layer material to a thickness of 5 nm. Thereafter, Compound ET-2 and Compound EI-1 were respectively introduced into two cells of the vacuum vapor deposition apparatus as electron transport layer materials, and the two materials were deposited at a weight ratio of 2:1 to form an electron transport layer with a thickness of 25 nm. After deposition of Yb (ytterbium) and LiF (lithium fluoride) at a weight ratio of 2:1 (Yb:LiF) on the electron transport layer to form an electron injection layer with a thickness of 1 nm, an Al cathode having a thickness of 80 nm was deposited on the electron injection layer by another vacuum vapor deposition apparatus. Thus, an OLED was produced. Each compound used for all of the materials was purified by vacuum sublimation at 10⁻⁶ Torr.

### Comparative Example 1: Producing an OLED Comprising a Conventional Compound as a Host Material

An OLED was produced in the same manner as in Device Example 1, except that the compounds shown in Table 1 below were used as host materials of the second light-emitting layer.

The driving voltage, current efficiency, and the time taken for luminance to decrease from 100% to 95% under 2× acceleration (lifespan; T₉₅) at a luminance of 1,000 nit of the OLEDs produced in Device Example 1 and Comparative Example 1 are provided in Table 1 below.

TABLE 1

| | | Driving | Current | |
|---|---|---|---|---|
| | Second Light- | Voltage | Efficiency | Lifespan |
| | Emitting Layer | [V] | [cd/A] | T₉₅ [hr] |
| Device | H1-206-D17:H2-666- | 3.5 | 6.2 | 122 |
| Example 1 | D13 | | | |
| Comparative | H-ref1-D19:H2-666- | 3.6 | 6.3 | 104 |
| Example 1 | D13 | | | |

From Table 1 above, it can be confirmed that the OLED (Device Example 1) comprising the specific combination of compounds according to the present disclosure as host materials exhibits longer lifespan characteristics compared to the OLED (Comparative Example 1) comprising the conventional compound.

### Device Examples 2 and 3: Producing OLEDs by Depositing the Compounds According to the Present Disclosure

OLEDs were produced in the same manner as in Device Example 1, except that the compounds shown in Table 2 below were used instead of Compound Host 1 in the first light-emitting layer.

The driving voltage and the time taken for luminance to decrease from 100% to 90% under 2× acceleration (lifespan; T₉₀) at a luminance of 1,000 nit of the OLEDs produced in Device Examples 2 and 3 are provided in Table 2 below.

TABLE 2

| | | | Driving | |
|---|---|---|---|---|
| | First Light- | Second Light- | Voltage | Lifespan |
| | Emitting Layer | Emitting Layer | [V] | T₉₀ [hr] |
| Device | H3-4 | H1-206-D17:H2-666- | 3.5 | 309 |
| Example 2 | | D13 | | |
| Device | H3-1 | H1-206-D17:H2-666- | 3.5 | 303 |
| Example 3 | | D13 | | |

From Table 2 above, it can be confirmed that the OLEDs (Device Examples 2 and 3) comprising the specific combination of compounds according to the present disclosure as host materials maintain a low driving voltage and exhibit longer lifespan characteristics.

The compounds used in Device Examples 1 to 3 and Comparative Example 1 above are specifically shown in the following Table 3.

TABLE 3

| | |
|---|---|
| Hole Injection Layer/Hole Transport Layer | 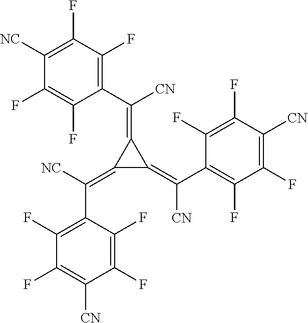 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 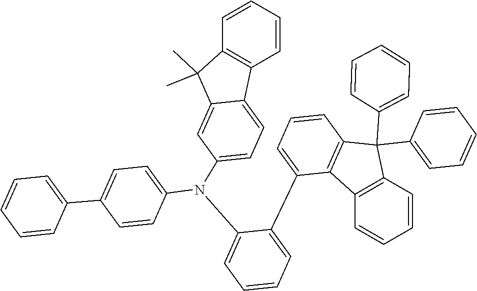 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-1 |
| | 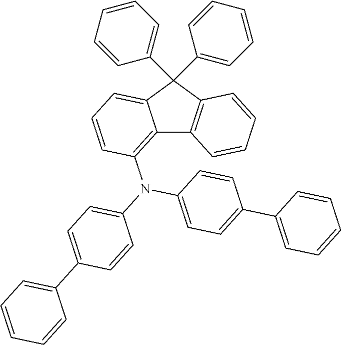 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT-2 |
| Light-Emitting Layer | 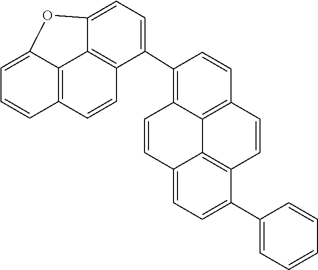 [SMILES: c1ccc(-c2ccc3ccc4c(-c5ccc6oc7cccc8ccc5c6c87)ccc5ccc2c3c54)cc1] |
| | Host 1 |
| | 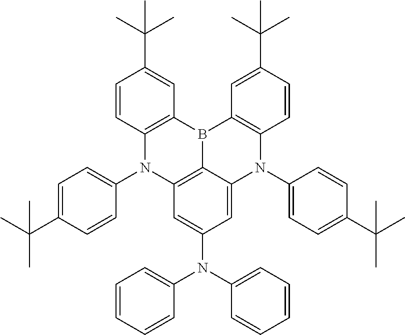 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1] |
| | D-1 |
| | 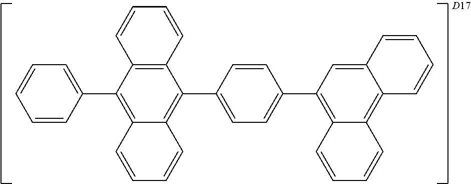 |
| | H1-206-D17 |
| | 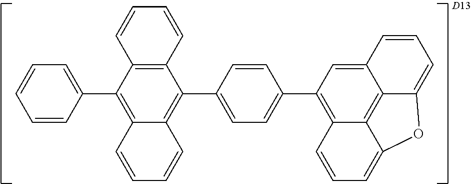 |
| | H2-666-D13 |
| | 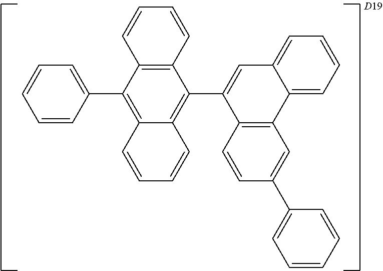 |
| | H-ref1-D19 |
| | 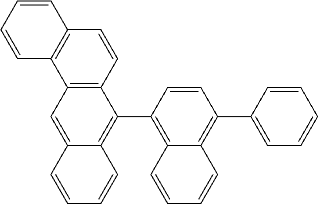 [SMILES: c1ccc(-c2ccc(-c3c4ccccc4cc4c3ccc3ccccc34)c3ccccc23)cc1] |
| | H3-1 |
| | 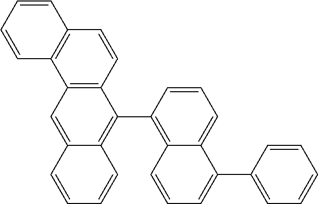 [SMILES: c1ccc(-c2cccc3c(-c4c5ccccc5cc5c4ccc4ccccc45)cccc23)cc1] |
| | H3-4 |
| Electron Transport Layer/Hole- Blocking Layer/ Electron Injection Layer | 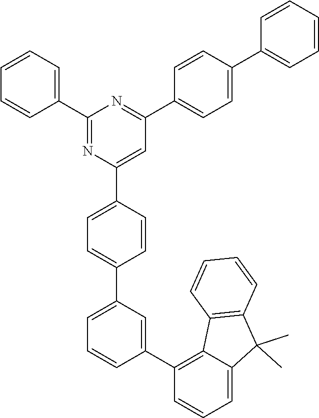 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 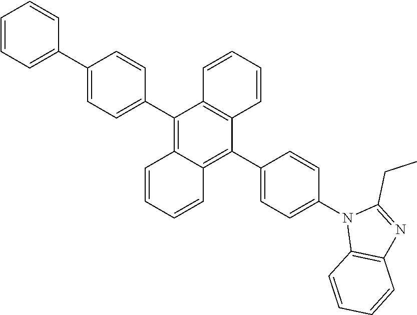 [SMILES: CCc1nc2ccccc2n1-c1ccc(-c2c3ccccc3c(-c3ccc(-c4ccccc4)cc3)c3ccccc23)cc1] |
| | ET-2 |
| | 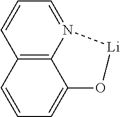 |
| | EI-1 |

### Device Examples 4 to 7: Producing OLEDs by Depositing the Compounds According to the Present Disclosure

OLEDs were produced in the same manner as in Device Example 1, except that the compounds shown in Table 4 below were used as host materials of the first light-emitting layer and the second light-emitting layer.

### Comparative Example 2: Producing an OLED Comprising a Single Light-Emitting Laver

An OLED was produced in the same manner as in Device Example 4, except that only the second light-emitting layer was deposited to a thickness of 18 nm without the first light-emitting layer.

The current efficiency, and the time taken for luminance to decrease from 100% to 95% under 2× acceleration (lifespan; T₉₅) at a luminance of 1,000 nit of the OLEDs produced in Device Examples 4 to 7 and Comparative Example 2 are

TABLE 4

| | | | Current | |
|---|---|---|---|---|
| | First Light- | Second Light- | Efficiency | Lifespan |
| | Emitting Layer | Emitting Layer | [cd/A] | T₉₅ [hr] |
| Device | H3-93-D18 | BH-1-D24:H2- | 6.0 | 102 |
| Example 4 | | 666-D13 | | |
| Device | H3-2 | BH-1-D24:H2- | 6.2 | 64 |
| Example 5 | | 666-D13 | | |
| Device | H3-11 | BH-1-D24:H2- | 5.8 | 78 |
| Example 6 | | 666-D13 | | |
| Device | H3-10 | BH-1-D24:H2- | 6.2 | 67 |
| Example 7 | | 666-D13 | | |
| Comparative | | BH-1-D24:H2- | 5.6 | 62 |
| Example 2 | | 666-D13 | | |

From Table 4 above, it can be confirmed that the OLEDs (Device Examples 4 to 7), comprising the specific combination of compounds according to the present disclosure as host materials, maintain high current efficiency and exhibit longer lifespan characteristics compared to the OLED (Comparative Example 2) comprising a single light-emitting layer.

The compounds used in Device Examples 4 to 7 and Comparative Example 2 above are specifically shown in the following Table 5.

TABLE 5

| | |
|---|---|
| Hole Injection Layer/Hole Transport Layer | 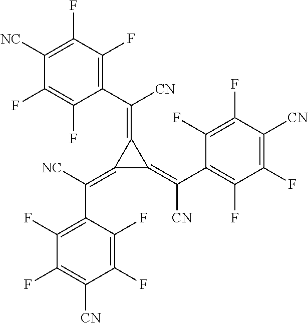 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 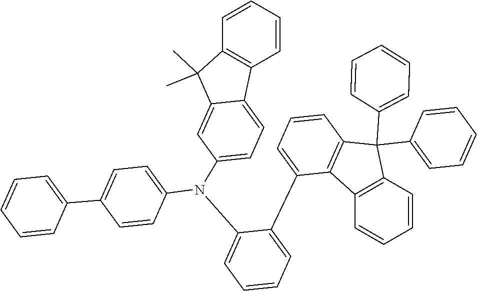 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-1 |
| | 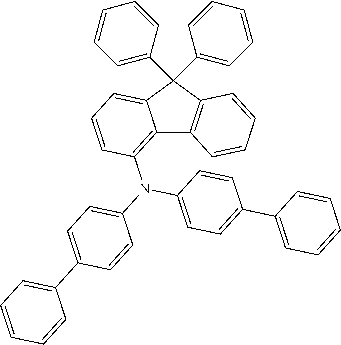 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT-2 |
| Light-Emitting Layer | 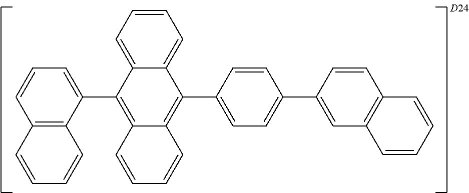 |
| | BH-1-D24 |
| | 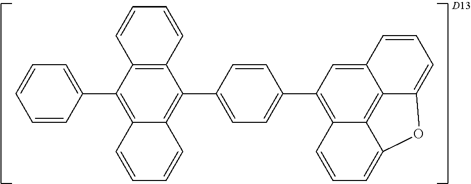 |
| | H2-666-D13 |
| | 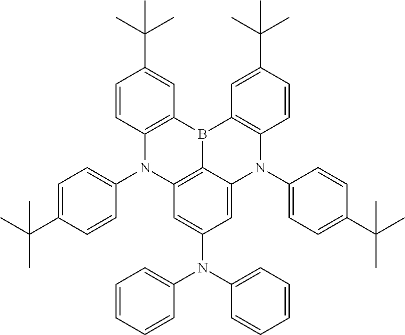 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1] |
| | D-1 |
| | 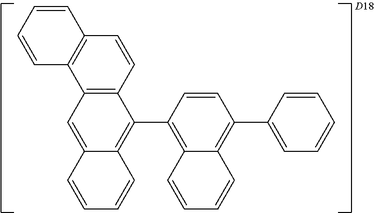 |
| | H3-93-D18 |
| | 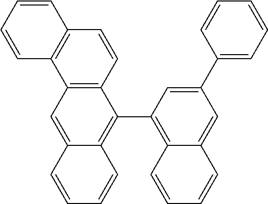 [SMILES: c1ccc(-c2cc(-c3c4ccccc4cc4c3ccc3ccccc34)c3ccccc3c2)cc1] |
| | H3-2 |
| | 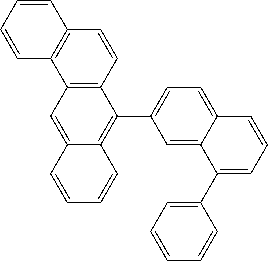 [SMILES: c1ccc(-c2cccc3ccc(-c4c5ccccc5cc5c4ccc4ccccc45)cc23)cc1] |
| | H3-11 |
| | 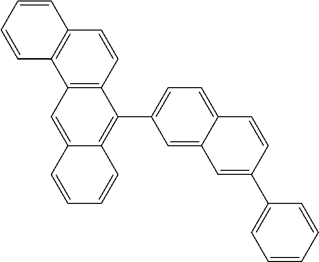 [SMILES: c1ccc(-c2ccc3ccc(-c4c5ccccc5cc5c4ccc4ccccc45)cc3c2)cc1] |
| | H3-10 |
| Electron Transport Layer/Hole- Blocking Layer/ Electron Injection Layer | 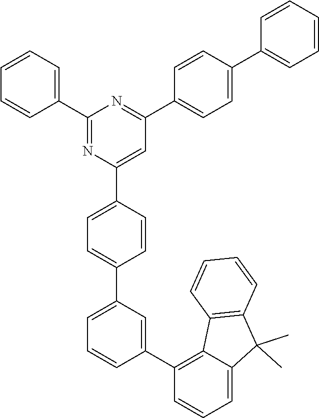 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 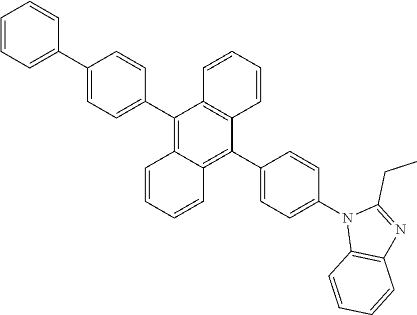 [SMILES: CCc1nc2ccccc2n1-c1ccc(-c2c3ccccc3c(-c3ccc(-c4ccccc4)cc3)c3ccccc23)cc1] |
| | ET-2 |
| | 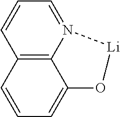 |
| | EI-1 |

### Device Example 8: Producing an OLED by Depositing the Compound According to the Present Disclosure

An OLED according to the present disclosure was produced. A transparent electrode indium tin oxide (ITO) thin film on a glass substrate for an OLED (GEOMATEC CO., LTD.) was subjected to an ultrasonic washing sequentially with acetone and isopropyl alcohol, and was then stored in isopropyl alcohol. The ITO substrate was then mounted on a substrate holder of a vacuum vapor deposition apparatus. Next, Compound HI-1 was introduced into a cell of the vacuum vapor deposition apparatus, and Compound HT-3 was introduced into another cell of the vacuum vapor deposition apparatus. The two materials were evaporated at different rates, and Compound HI-1 was deposited in a doping amount of 5 wt % based on the total amount of Compound HI-1 and Compound HT-3 to form a hole injection layer having a thickness of 10 nm. Compound HT-3 was then deposited on the hole injection layer to form a first hole transport layer having a thickness of 80 nm. Compound HT-4 was then introduced into another cell of the vacuum vapor deposition apparatus and was evaporated by applying an electric current to the cell to thereby form a second hole transport layer having a thickness of 15 nm on the first hole transport layer. After formation of the hole injection layer and the hole transport layers, a light-emitting layer was formed thereon as follows. The compound shown in Table 6 below was introduced into a cell of the vacuum vapor deposition apparatus as a host, and Compound D-2 was introduced into another cell as a dopant. The two materials were evaporated at different rates, and the dopant was deposited in a doping amount of 3 wt % based on the total amount of the host and dopant to form a light-emitting layer with a thickness of 20 nm on the second hole transport layer. After deposition of the light-emitting layer, Compound ET-3 was deposited as a hole-blocking layer material to a thickness of 5 nm. Thereafter, Compound ET-4 and Compound EI-1 were respectively introduced into two cells of the vacuum vapor deposition apparatus as electron transport layer materials, and the two materials were deposited at a weight ratio of 2:1 to form an electron transport layer with a thickness of 25 nm. After deposition of Yb (ytterbium) on the electron transport layer to form an electron injection layer with a thickness of 2 nm, an Al cathode having a thickness of 80 nm was deposited on the electron injection layer by another vacuum vapor deposition apparatus. Thus, an OLED was produced. Each compound used for all of the materials was purified by vacuum sublimation at 10⁻⁶ Torr.

### Comparative Example 3: Producing an OLED Comprising a Comparative Compound

An OLED was produced in the same manner as in Device Example 8, except that the compound shown in Table 6 below was used as a host material.

The driving voltage, and the time taken for luminance to decrease from 100% to 95% under 2× acceleration (lifespan; T₉₅) at a luminance of 1,000 nit of the OLEDs produced in Device Example 8 and Comparative Example 3 are provided in Table 6 below.

TABLE 6

| | | Driving | |
|---|---|---|---|
| | | Voltage | Lifespan |
| | Light-Emitting Layer | [V] | T₉₅ [hr] |
| Device | H2-839-D12 | 3.1 | 90 |
| Example 8 | | | |
| Comparative | H2-491-D14 | 3.2 | 51 |
| Example 3 | | | |

From Table 6 above, it can be confirmed that the OLED (Device Example 8) comprising the compound according to the present disclosure as a host material F183914-US-NP maintains low driving voltage and exhibits longer lifespan characteristics compared to the OLED (Comparative Example 3) comprising the comparative compound.

The compounds used in Device Example 8 and Comparative Example 3 above are specifically shown in the following Table 7.

TABLE 7

| | |
|---|---|
| Hole Injection Layer/Hole Transport Layer | 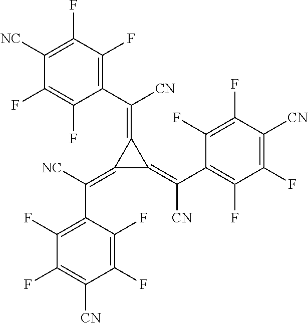 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 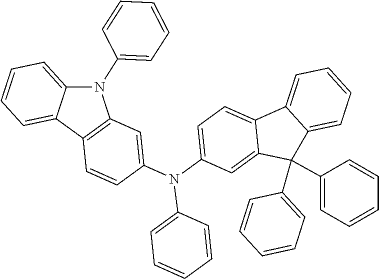 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-3 |
| | 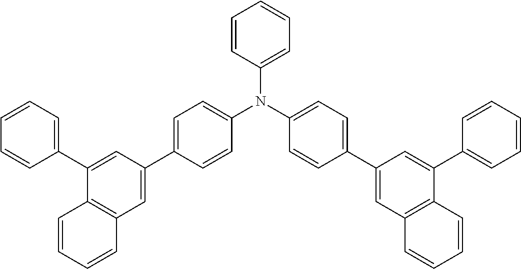 [SMILES: c1ccc(-c2cc(-c3ccc(N(c4ccccc4)c4ccc(-c5cc(-c6ccccc6)c6ccccc6c5)cc4)cc3)cc3ccccc23)cc1] |
| | HT-4 |
| Light-Emitting Layer | 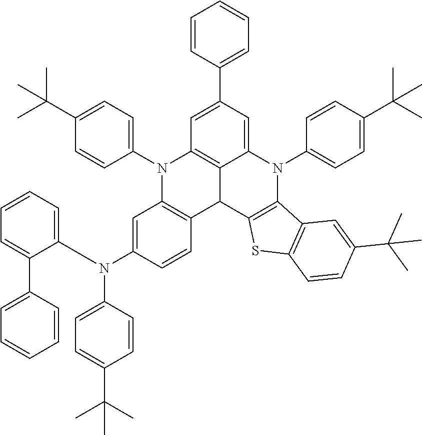 [SMILES: CC(C)(C)c1ccc(N(c2ccc3c(c2)N(c2ccc(C(C)(C)C)cc2)c2cc(-c4ccccc4)cc4c2C3c2sc3ccc(C(C)(C)C)cc3c2N4c2ccc(C(C)(C)C)cc2)c2ccccc2-c2ccccc2)cc1] |
| | D-2 |
| | 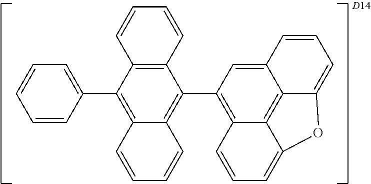 |
| | H2-491-D14 |
| | 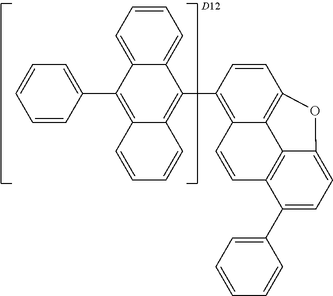 |
| | H2-839-D12 |
| Electron Transport Layer/Hole- Blocking Layer/ Electron Injection Layer | 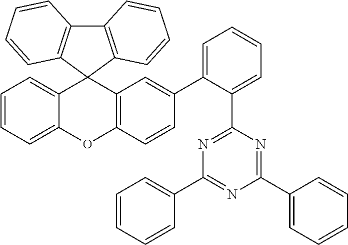 [SMILES: c1ccc(-c2nc(-c3ccccc3)nc(-c3ccccc3-c3ccc4c(c3)C3(c5ccccc5O4)c4ccccc4-c4ccccc43)n2)cc1] |
| | ET-3 |
| | 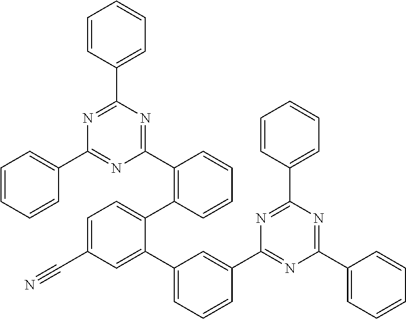 [SMILES: N#Cc1ccc(-c2ccccc2-c2nc(-c3ccccc3)nc(-c3ccccc3)n2)c(-c2cccc(-c3nc(-c4ccccc4)nc(-c4ccccc4)n3)c2)c1] |
| | ET-4 |
| | 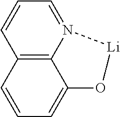 |
| | EI-1 |

1. A plurality of host materials comprising at least one first host compound and at least one second host compound, wherein the first host compound is represented by the following Formula 1, the second host compound is represented by the following Formula 2, and the first host compound and the second host compound are different from each other: 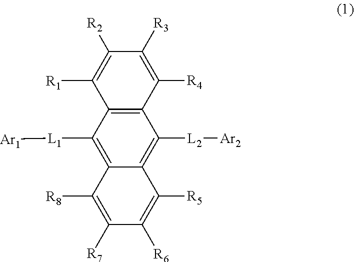
wherein,
Ar₁ represents a substituted or unsubstituted (C₆-C₃₀)aryl or a substituted or unsubstituted (3- to 30-membered)heteroaryl;
L₁ represents a single bond or a substituted or unsubstituted (C₆-C₃₀)arylene;
R₁ to R₈ each independently represent hydrogen or deuterium;
L₂ represents a substituted or unsubstituted (C₆-C₃₀)arylene; and
Ar₂ is represented by the following Formula 1-1: 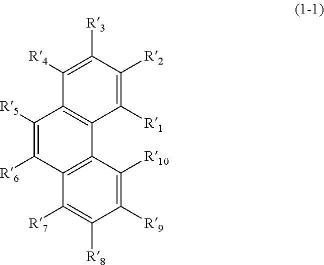
wherein,
R′₁ to R′₁₀ each independently represent hydrogen, deuterium, or a (C₆-C₃₀)aryl unsubstituted or substituted with deuterium, and one of R′₁ to R′₁₀ is linked to L₂; 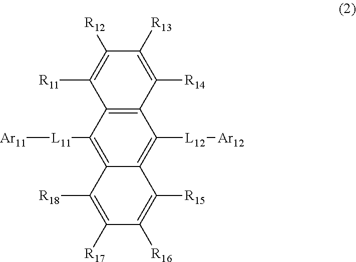
wherein
Ar₁₁ represents a substituted or unsubstituted (C₆-C₃₀)aryl or a substituted or unsubstituted (3- to 30-membered)heteroaryl;
L₁₁ represents a single bond, a substituted or unsubstituted (C₆-C₃₀)arylene, or a substituted or unsubstituted (3- to 30-membered)heteroarylene;
R₁₁ to R₁₈ each independently represent hydrogen or deuterium;
L₁₂ represents a single bond or a substituted or unsubstituted (C₆-C₃₀)arylene; and
Ar₁₂ is represented by the following Formula 2-1: 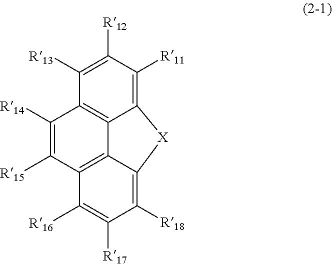
wherein,
X is O or S;
R′₁₁ to R′₁₈ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a combination thereof; or may be linked to the adjacent substituents to form a ring(s); and
one of R′₁₁ to R′₁₈ is linked to L₁₂.

2. The plurality of host materials according to claim 1, wherein Ar₁ represents a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof;
L₁ and L₂ each independently represent a single bond, a phenylene unsubstituted or substituted with deuterium, a biphenylene unsubstituted or substituted with deuterium, a terphenylene unsubstituted or substituted with deuterium, or a naphthylene unsubstituted or substituted with deuterium; and
R′₁ to R′₁₀ each independently represent hydrogen, deuterium, a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof.

3. The plurality of host materials according to claim 1, wherein Ar₁₁ represents a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, a dimethylfluorenyl unsubstituted or substituted with deuterium, a diphenylfluorenyl unsubstituted or substituted with deuterium, a dimethylbenzofluorenyl unsubstituted or substituted with deuterium, a diphenylbenzofluorenyl unsubstituted or substituted with deuterium, a spirobifluorenyl unsubstituted or substituted with deuterium, a triphenylenyl unsubstituted or substituted with deuterium, a carbazolyl unsubstituted or substituted with deuterium, a benzocarbazolyl unsubstituted or substituted with deuterium, a dibenzofuranyl unsubstituted or substituted with deuterium, a benzonaphthofuranyl unsubstituted or substituted with deuterium, a dibenzothiophenyl unsubstituted or substituted with deuterium, a benzonaphthothiophenyl unsubstituted or substituted with deuterium, or a combination thereof;
L₁₁ and L₁₂ each independently represent a single bond, a phenylene unsubstituted or substituted with deuterium, a biphenylene unsubstituted or substituted with deuterium, a terphenylene unsubstituted or substituted with deuterium, a naphthylene unsubstituted or substituted with deuterium, or a carbazolylene unsubstituted or substituted with deuterium; and
R′₁₁ to R′₁₈ each independently represent hydrogen, deuterium, a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof; or may be linked to the adjacent substituents to form a benzene ring(s) unsubstituted or substituted with deuterium.

4. The plurality of host materials according to claim 1, wherein at least one of Formulas 1 and 2 comprises deuterium.

5. The plurality of host materials according to claim 1, wherein the substituted alkyl, the substituted aryl, the substituted arylene, the substituted heteroaryl, the substituted heteroarylene, the substituted cycloalkyl, the substituted fused ring group of an aliphatic ring(s) and an aromatic ring(s), the substituted arene, and the substituted heteroarene are each independently substituted with at least one selected from the group consisting of deuterium, a halogen, a cyano, carboxyl, nitro, hydroxyl, phosphine oxide group, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (3- to 30-membered)heteroaryl, (C₆-C₃₀)aryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, amino, mono- or di(C₁-C₃₀)alkylamino, mono- or di(C₂-C₃₀)alkenylamino, mono- or di(C₆-C₃₀)arylamino, mono- or di(3- to 30-membered)heteroarylamino, (C₁-C₃₀)alkyl(C₂-C₃₀)alkenylamino, (C₁-C₃₀)alkyl(C₆-C₃₀)arylamino, (C₁-C₃₀)alkyl(3- to 30-membered)heteroarylamino, (C₂-C₃₀)alkenyl(C₆-C₃₀)arylamino, (C₂-C₃₀)alkenyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, di(C₆-C₃₀)arylboronyl, (C₆-C₃₀)arylphosphinyl, di(C₁-C₃₀)alkylboronyl, (C₁-C₃₀)alkyl(C₆-C₃₀)arylboronyl, (C₆-C₃₀)ar(C₁-C₃₀)alkyl, (C₁-C₃₀)alkyl(C₆-C₃₀)aryl, and a combination thereof, each of which may be further substituted with deuterium.

6. The plurality of host materials according to claim 1, wherein the compound represented by Formula 1 is at least one selected from the following compounds: 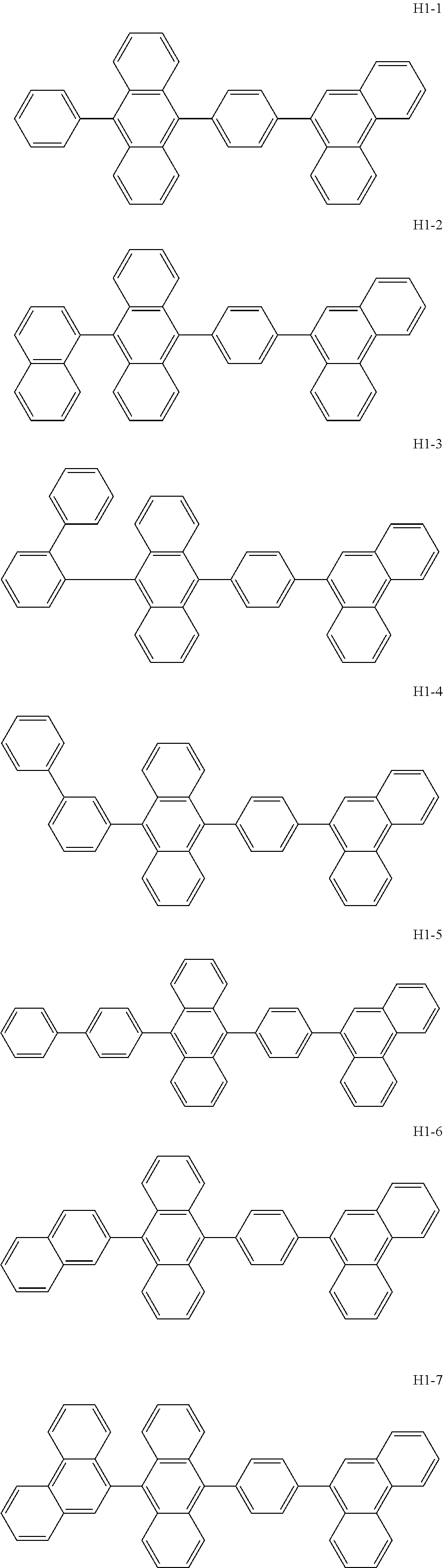 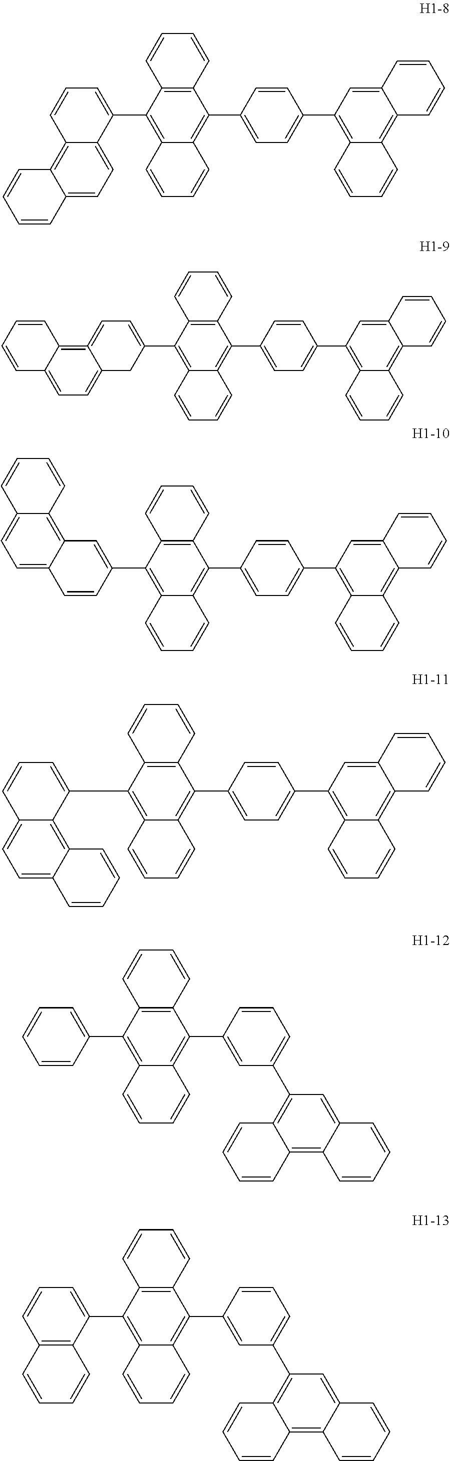 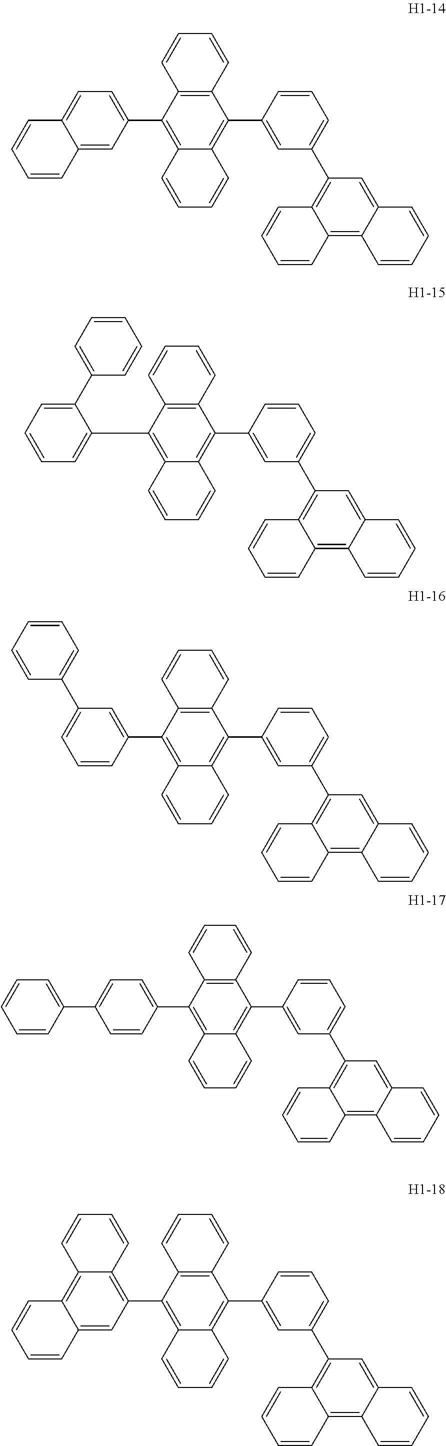 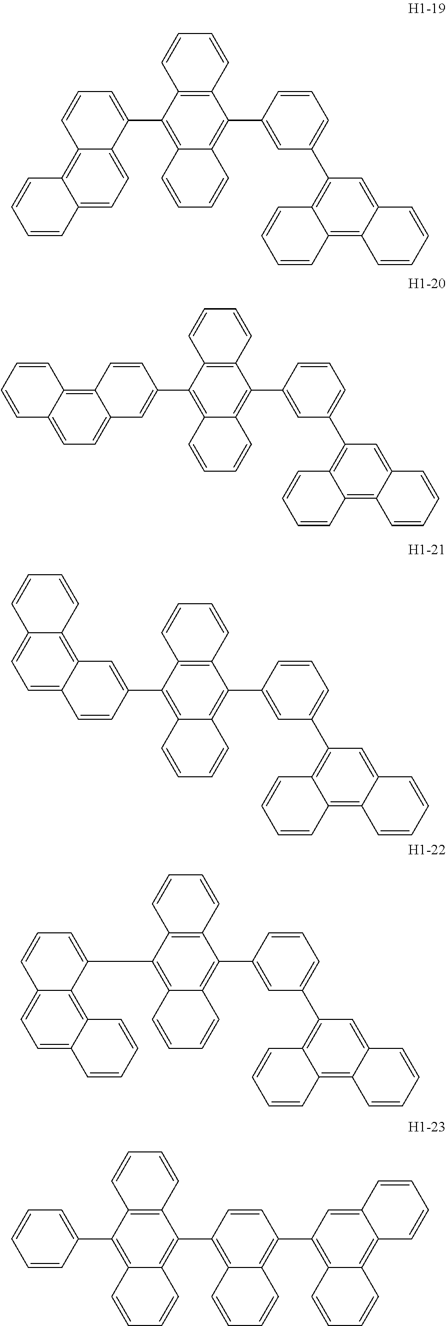 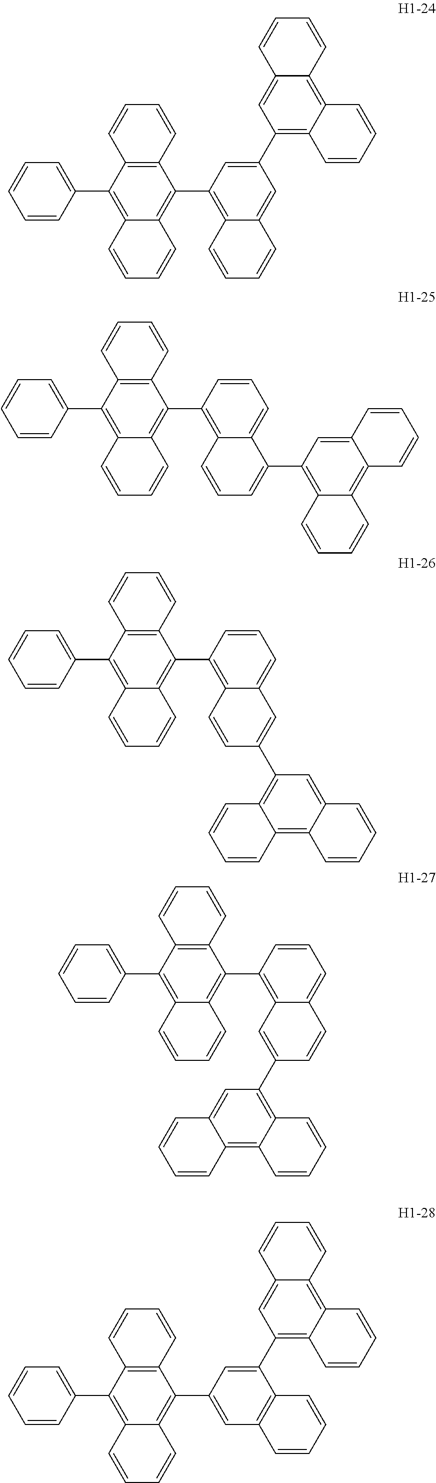 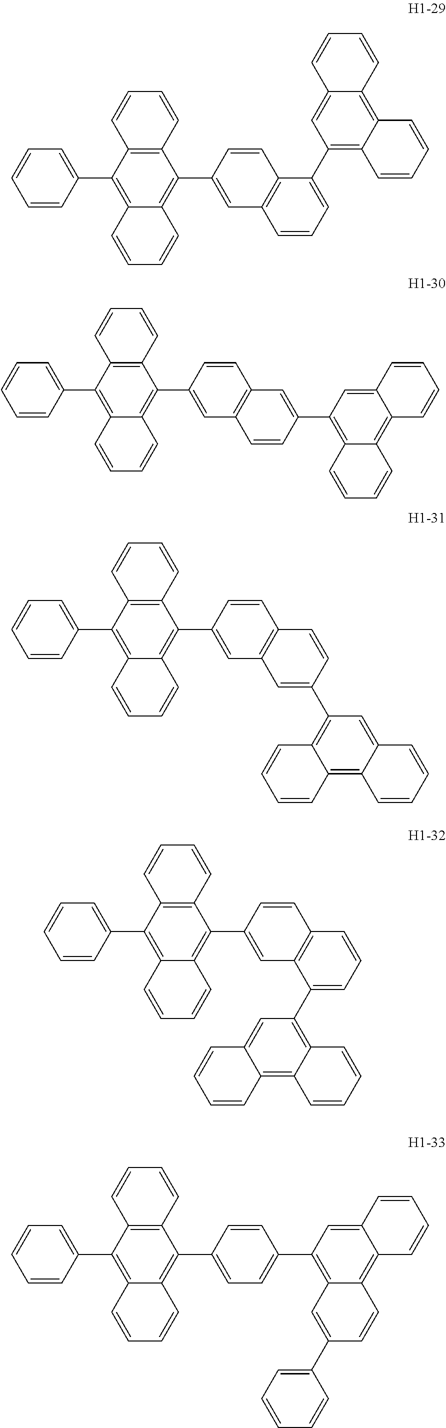 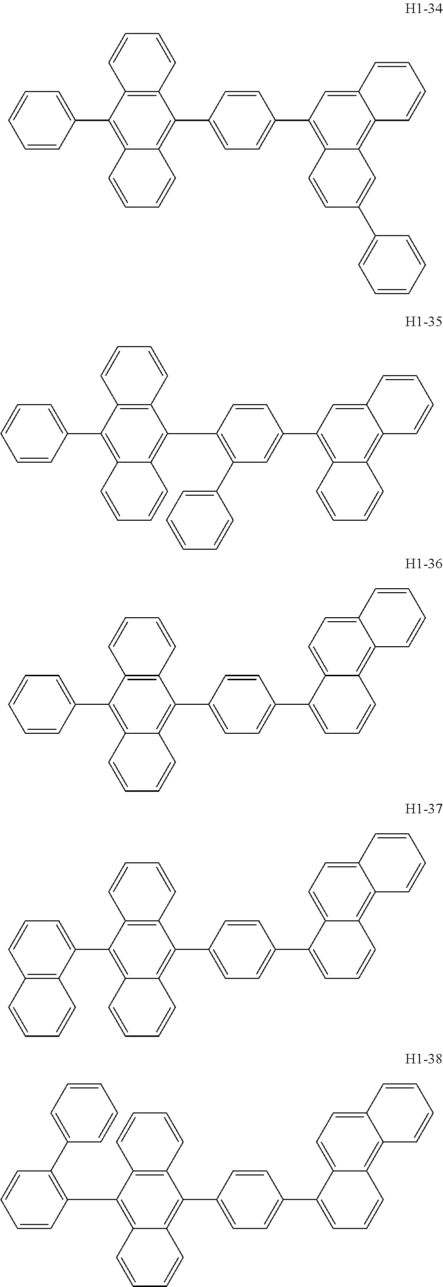 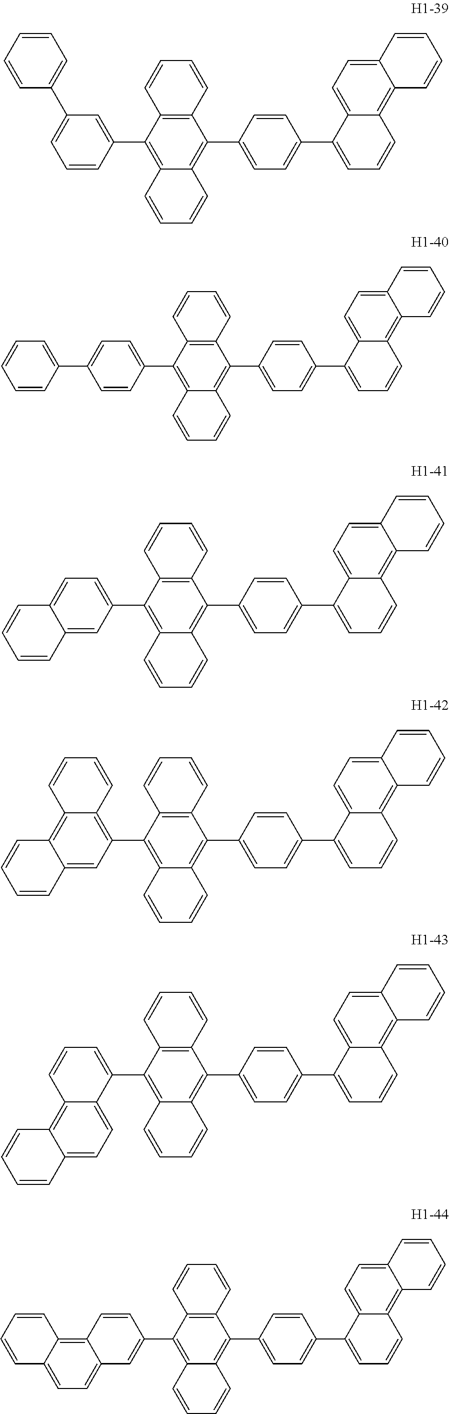 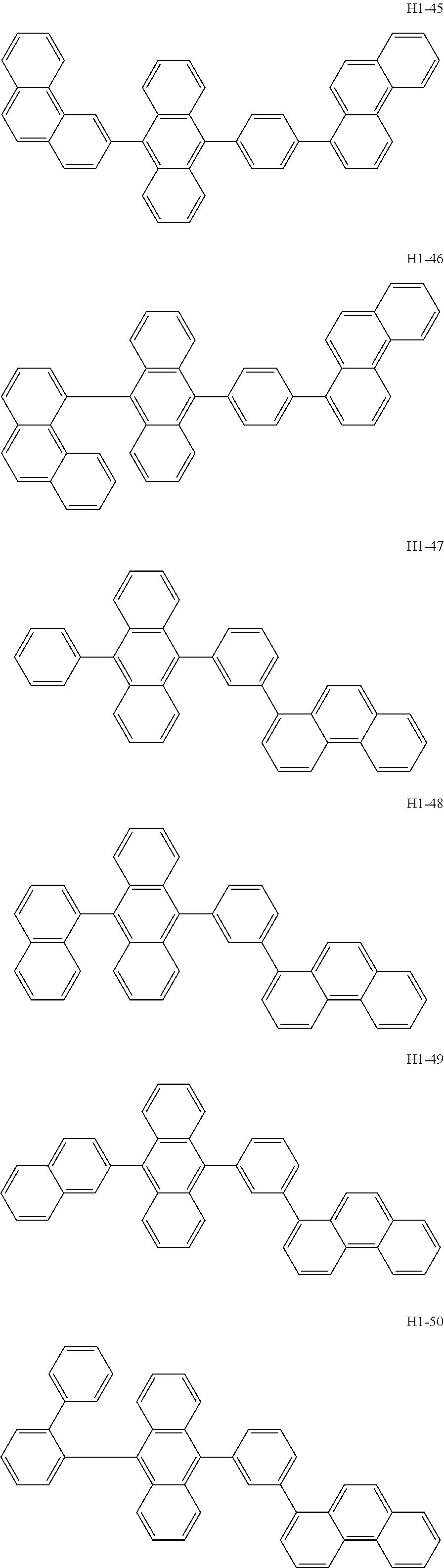 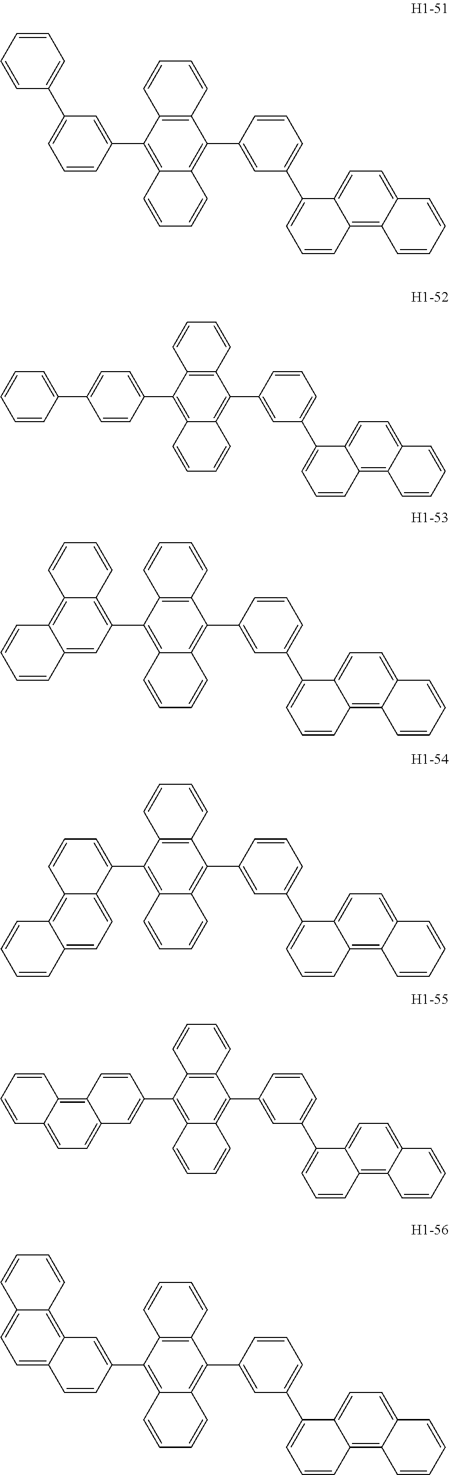 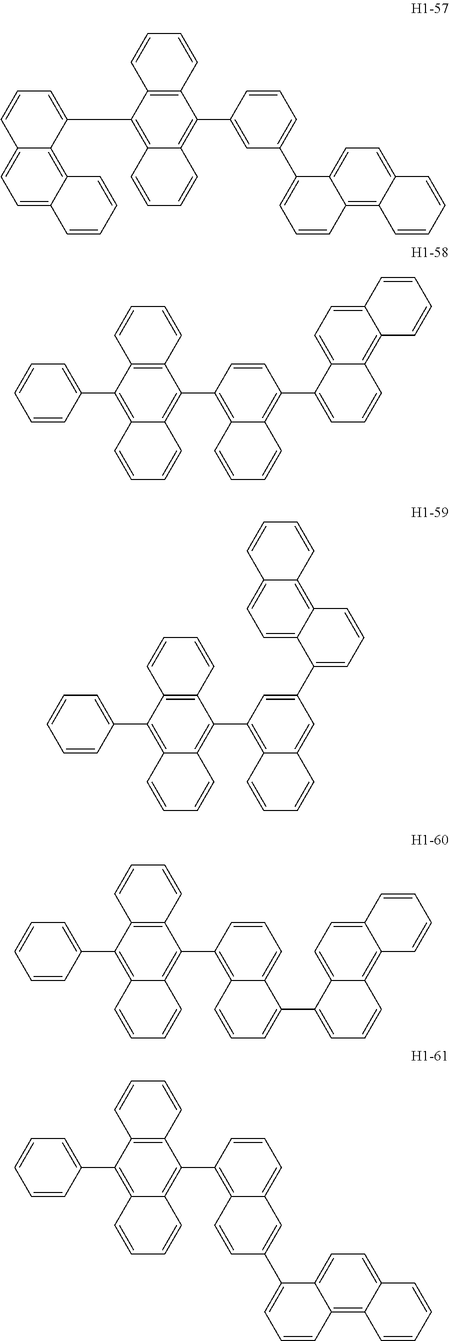 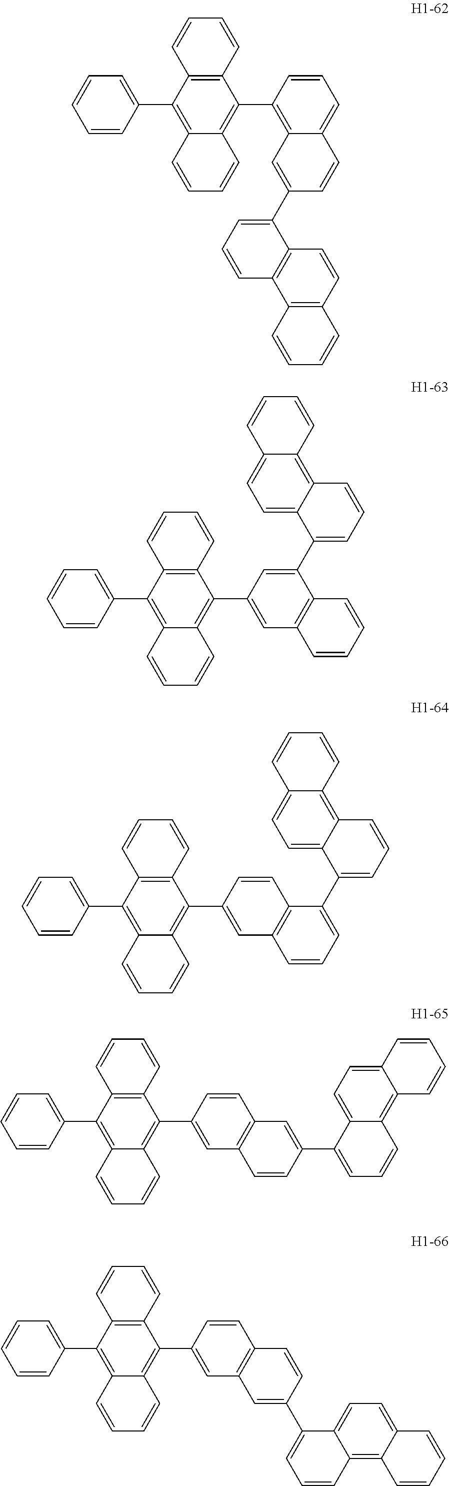 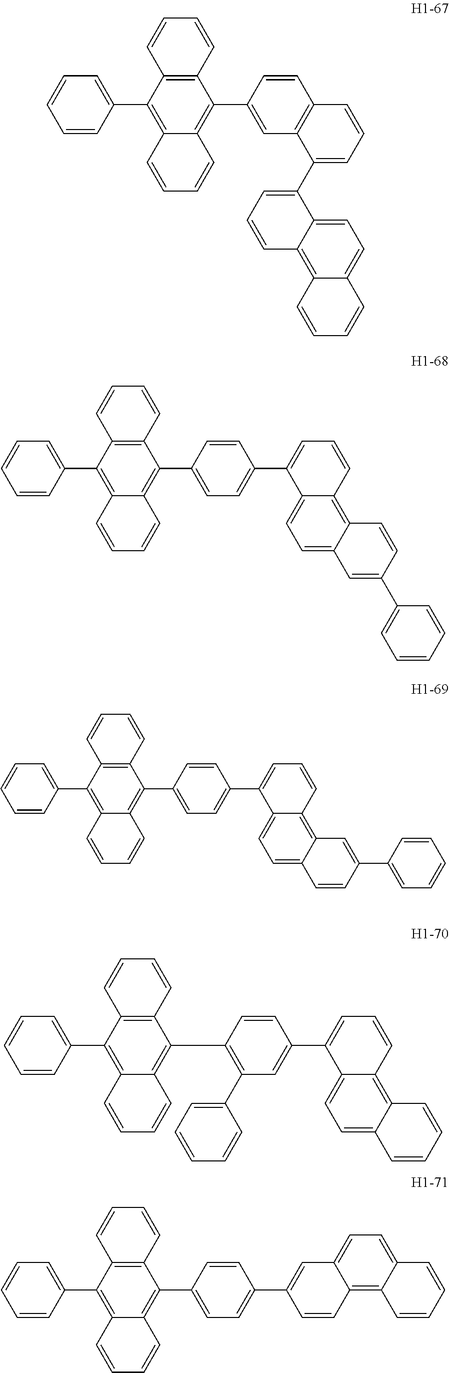 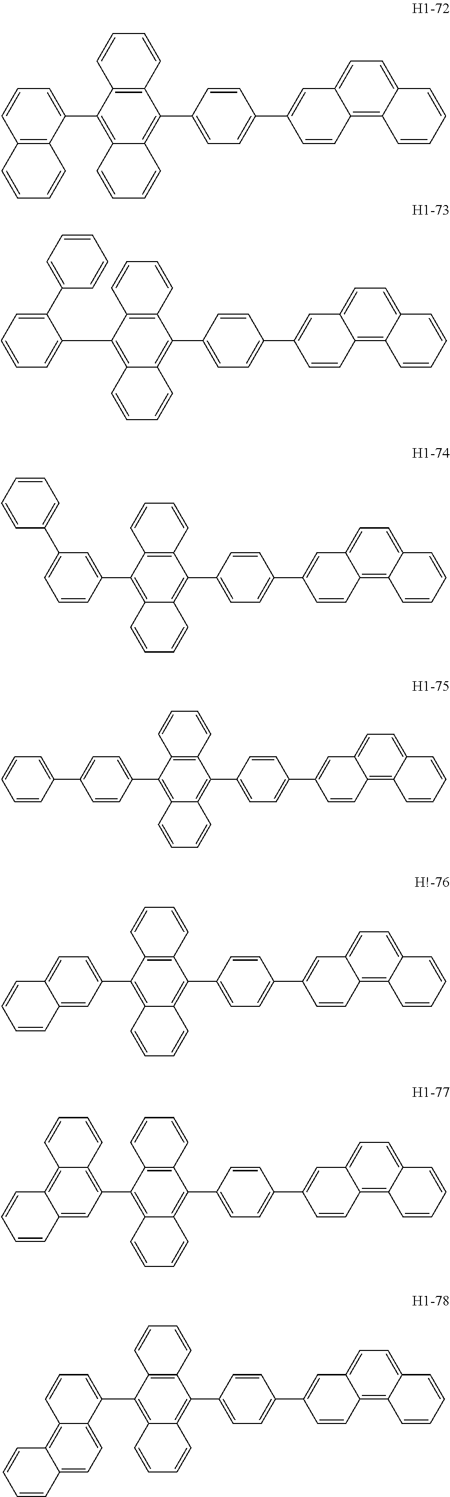 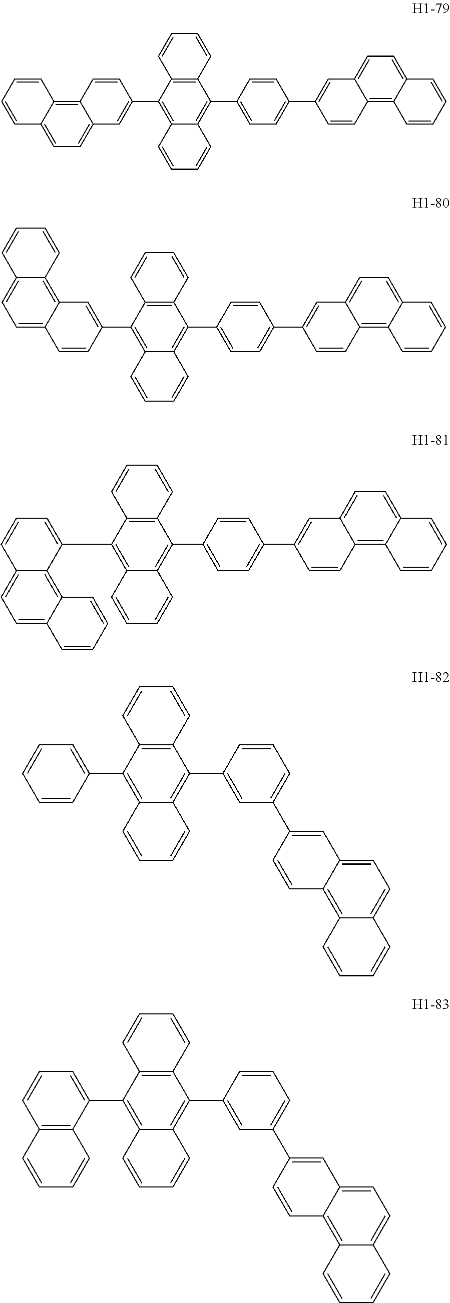 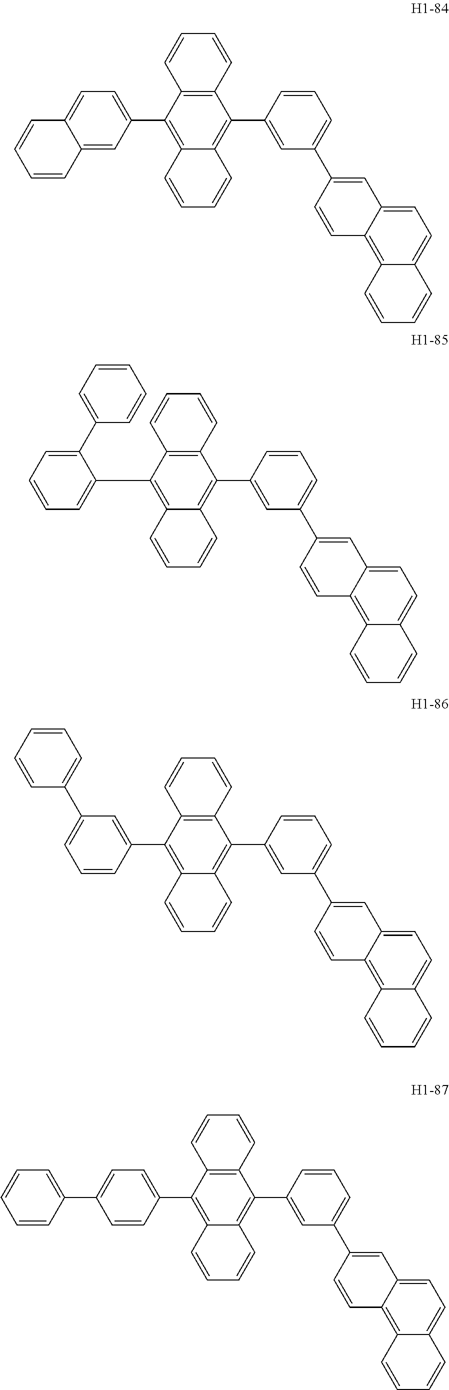 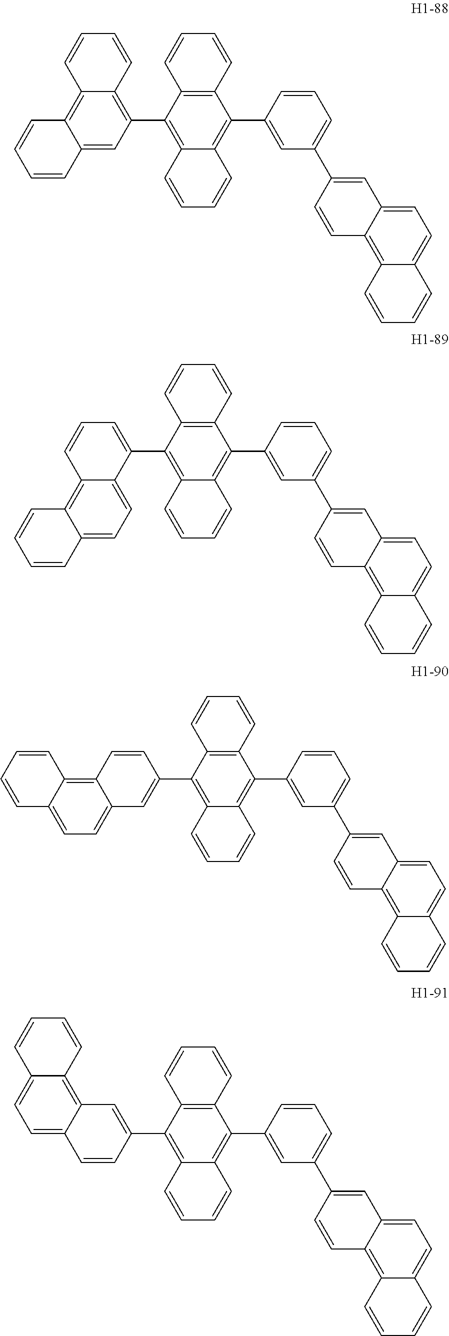 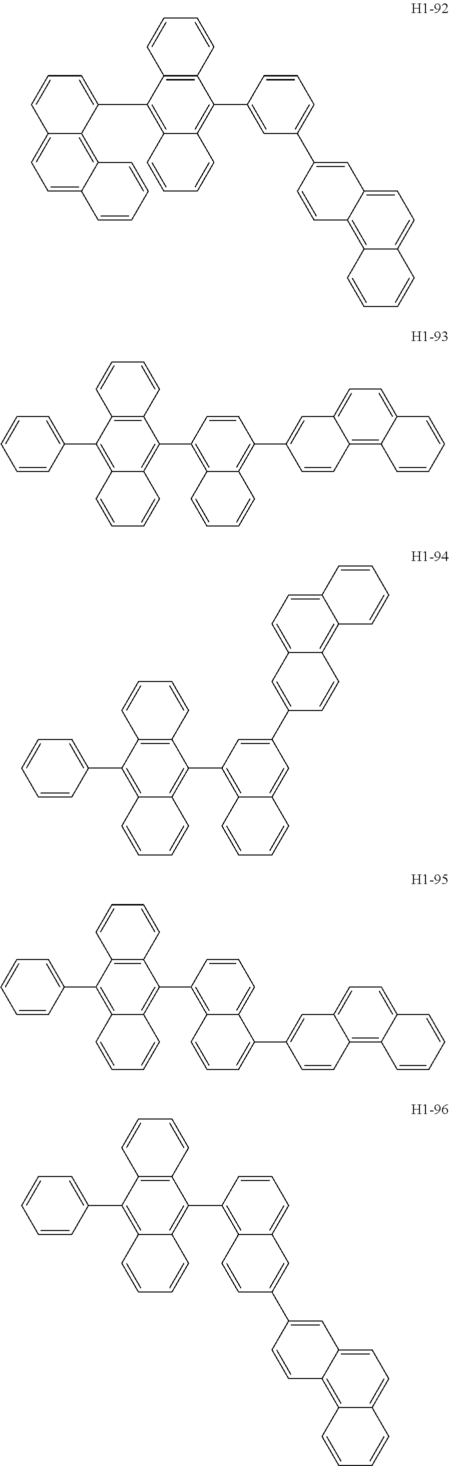 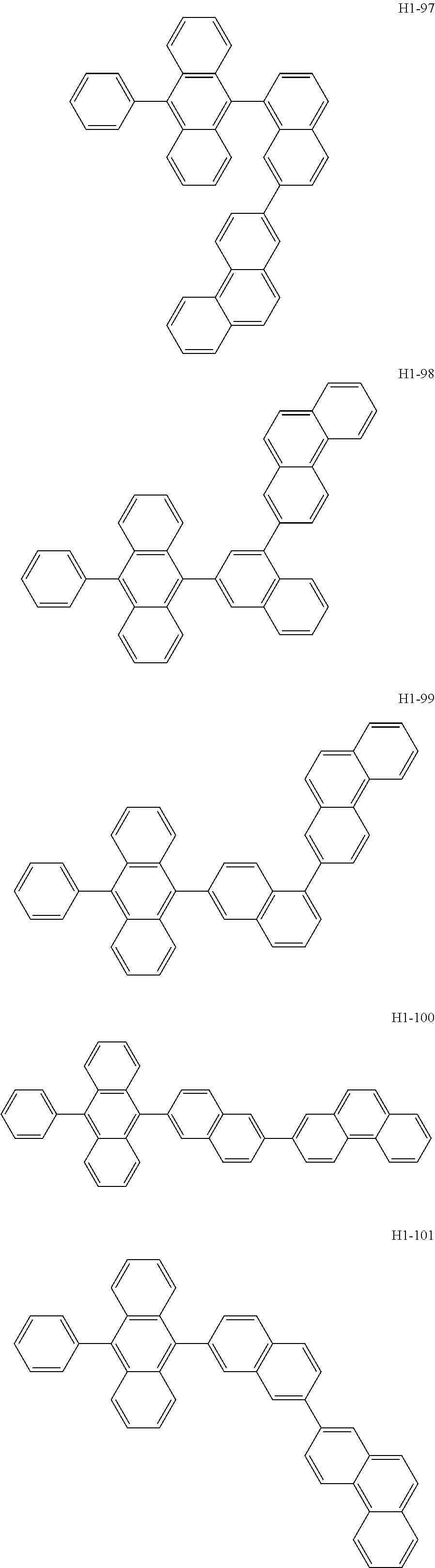 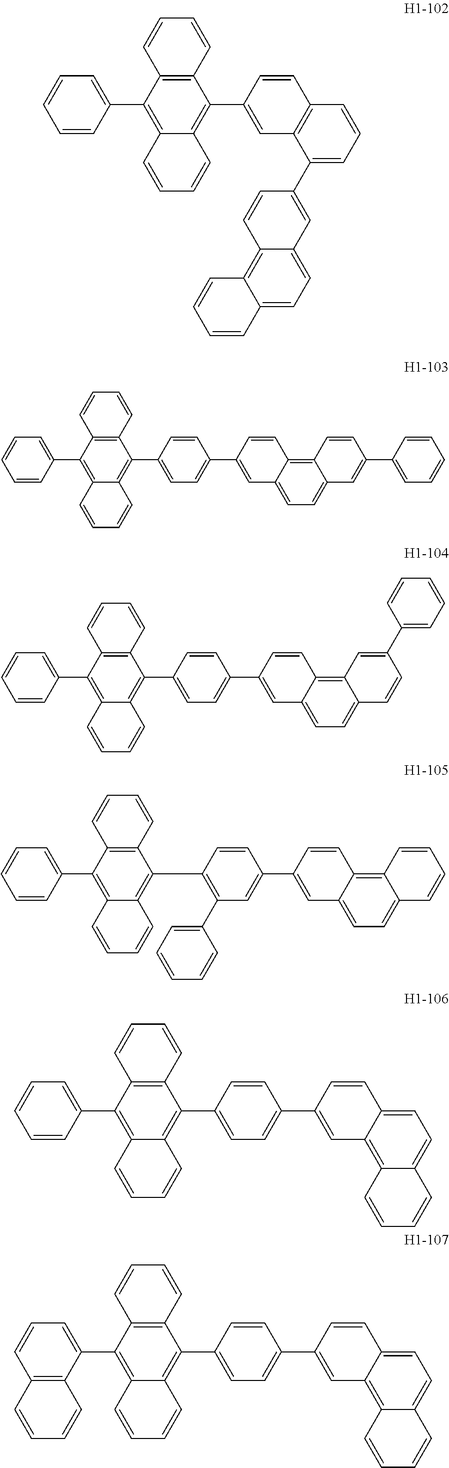 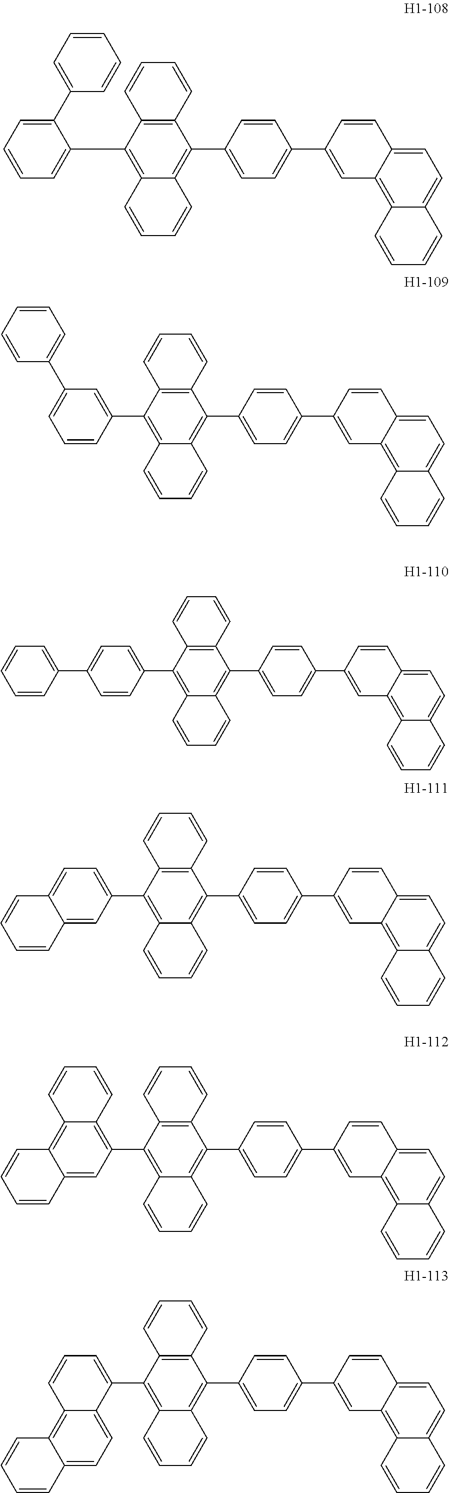 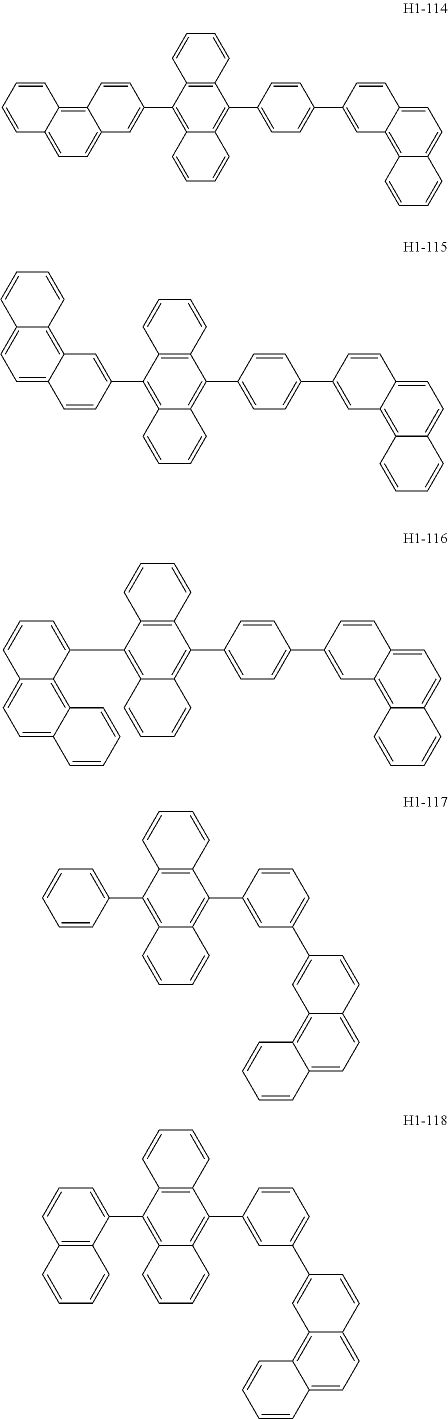 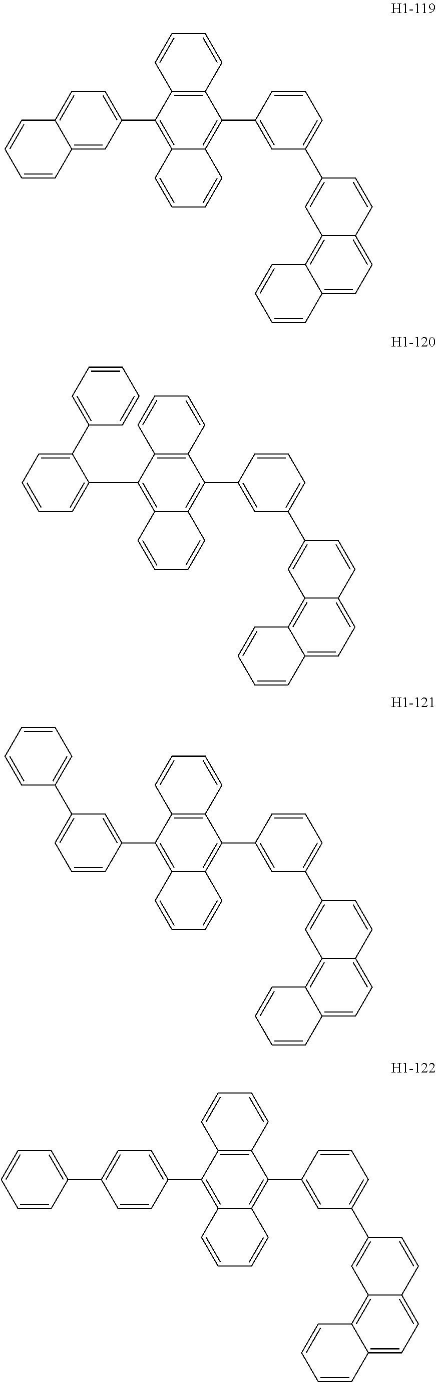 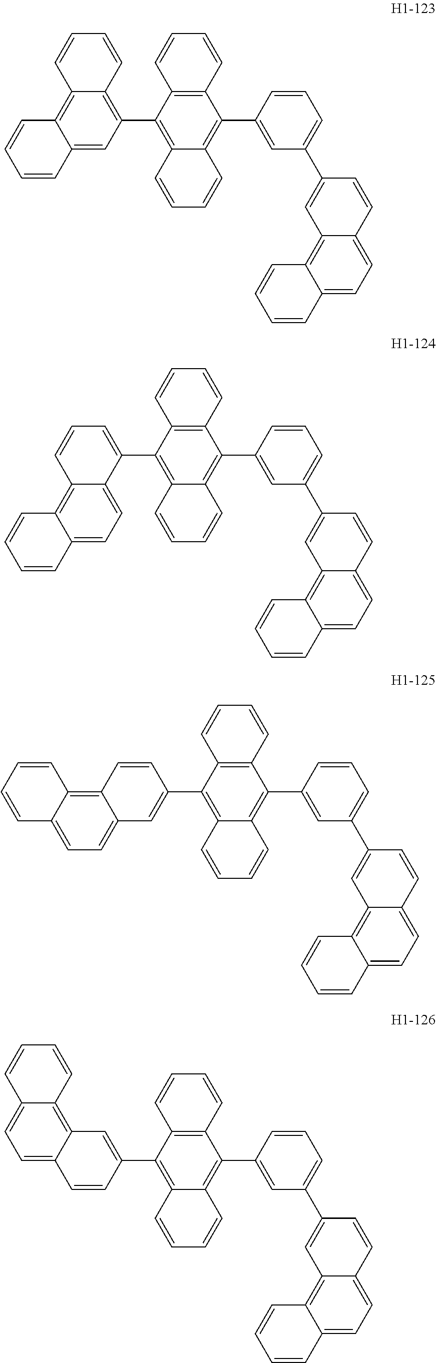 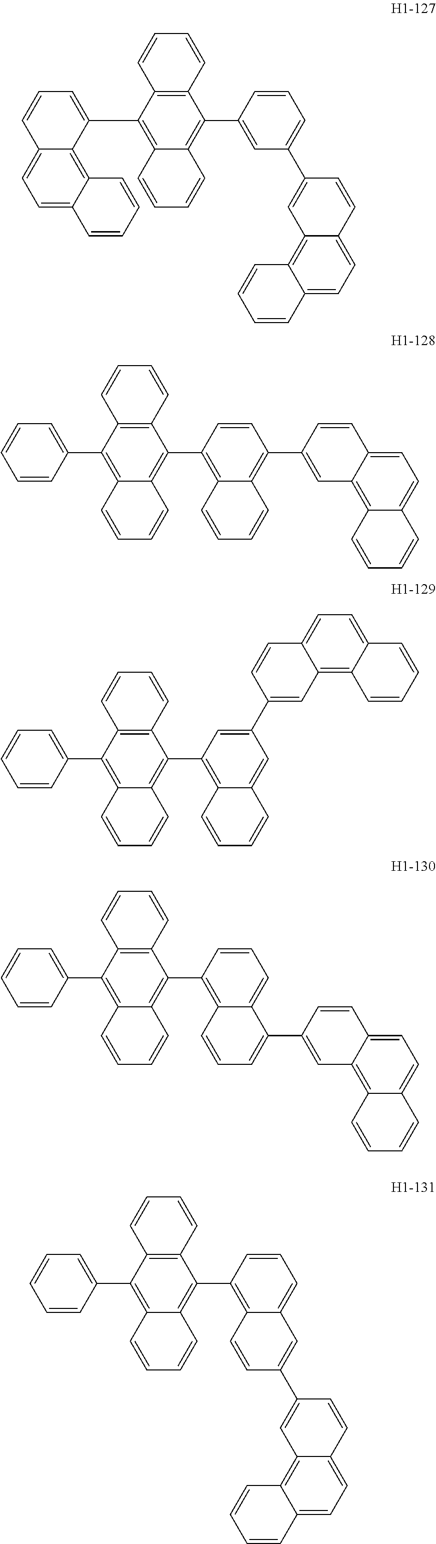 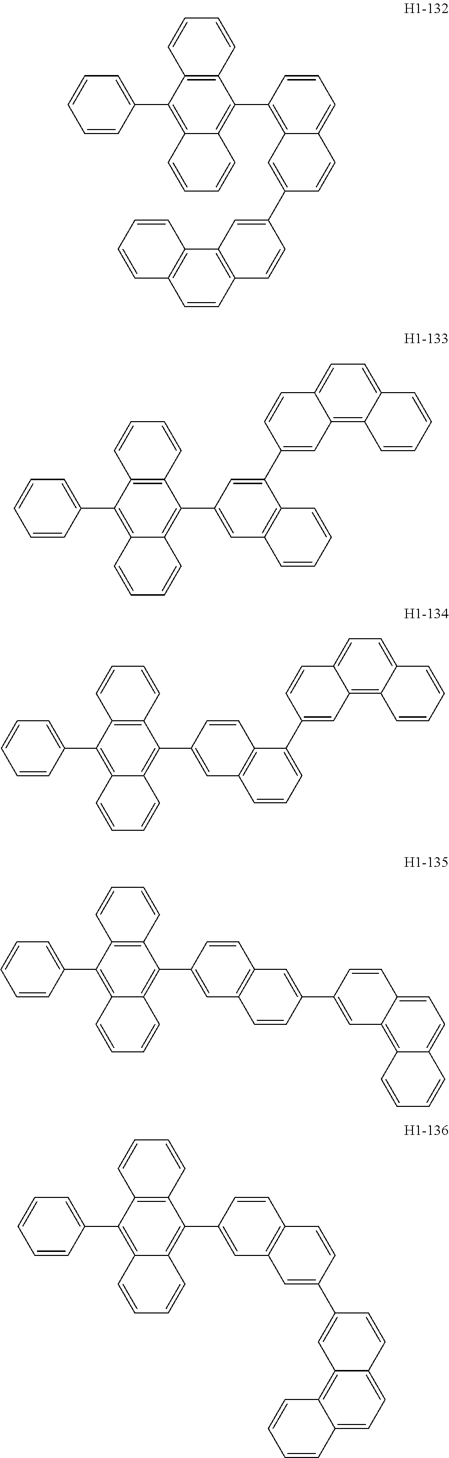 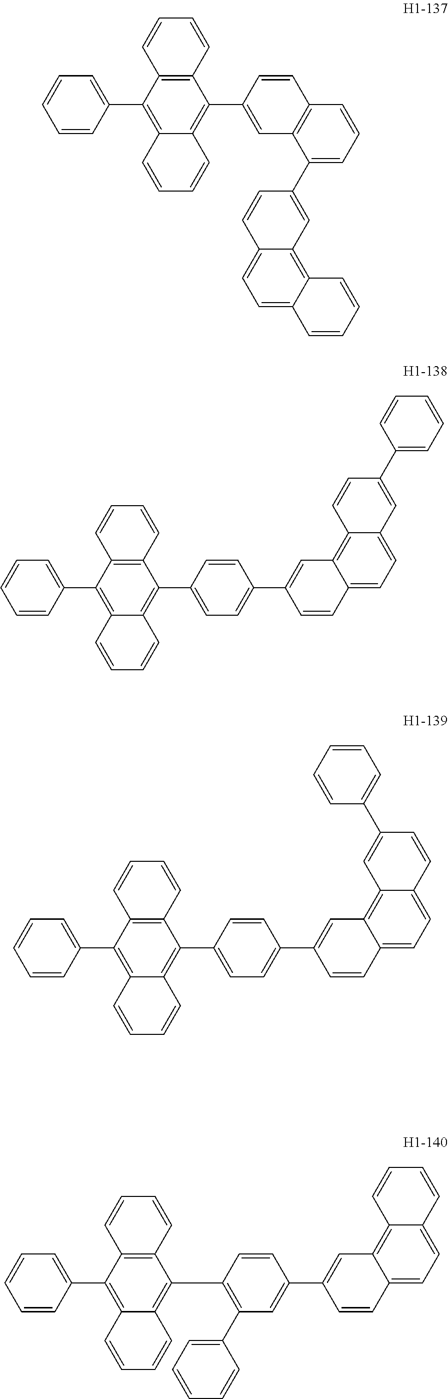 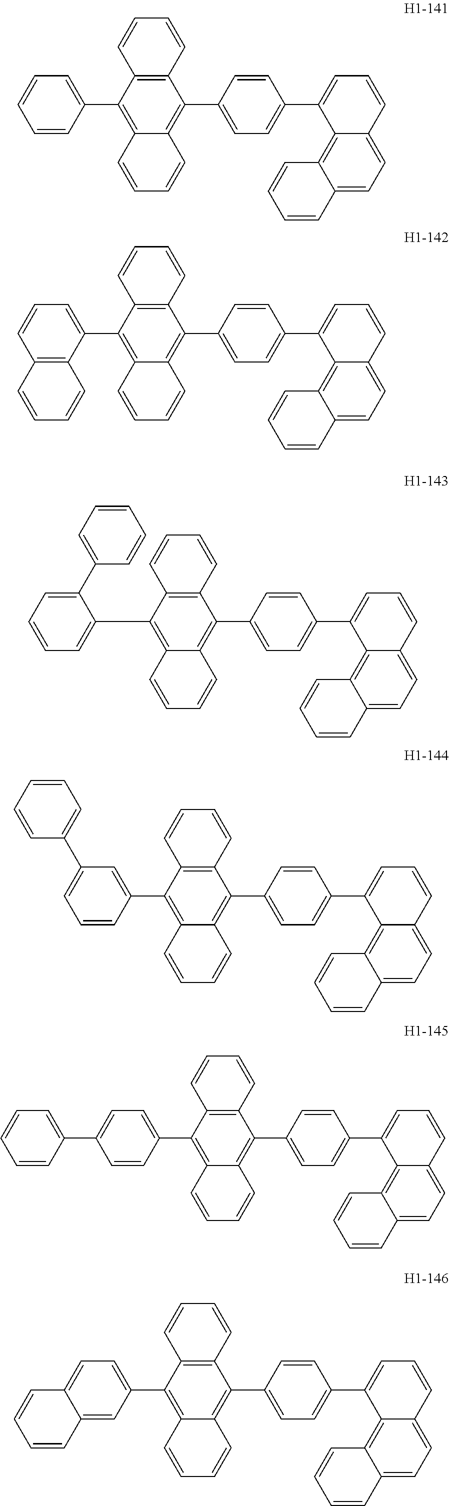 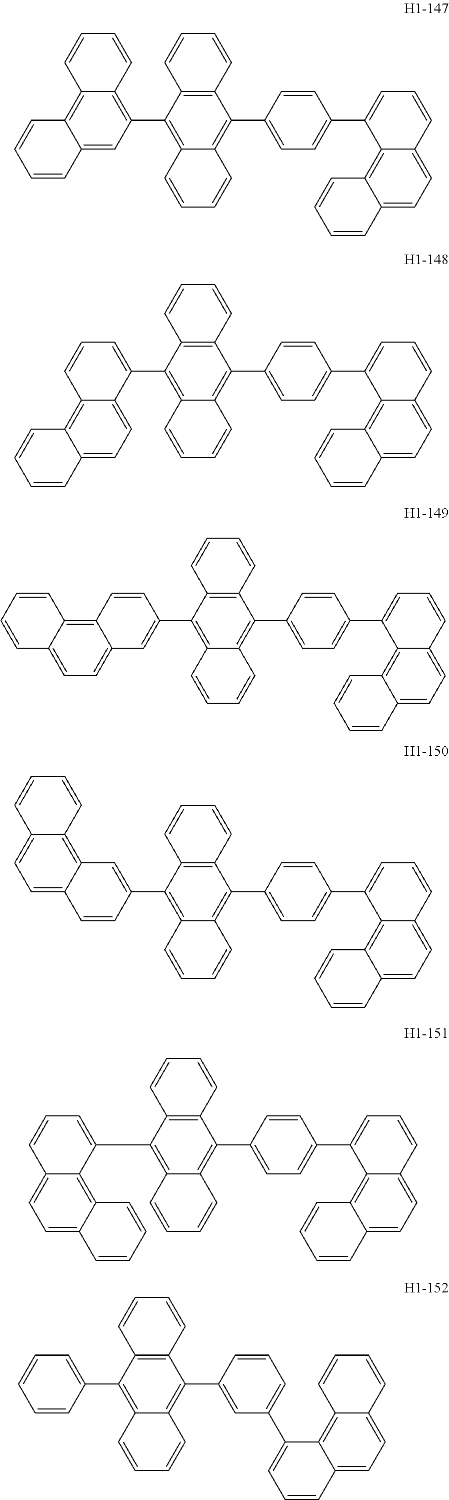 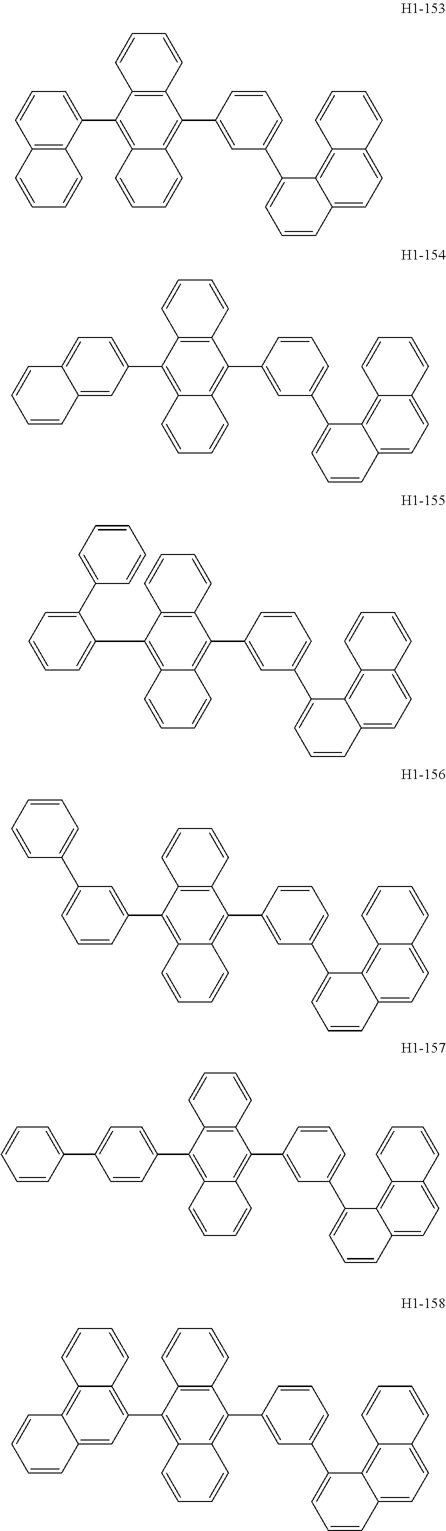 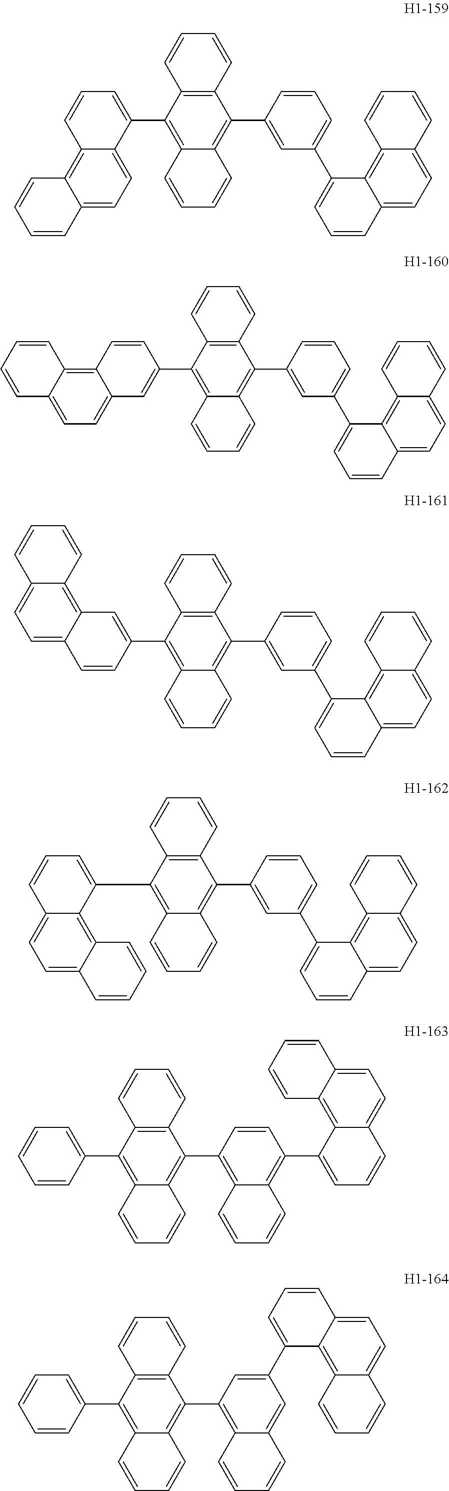 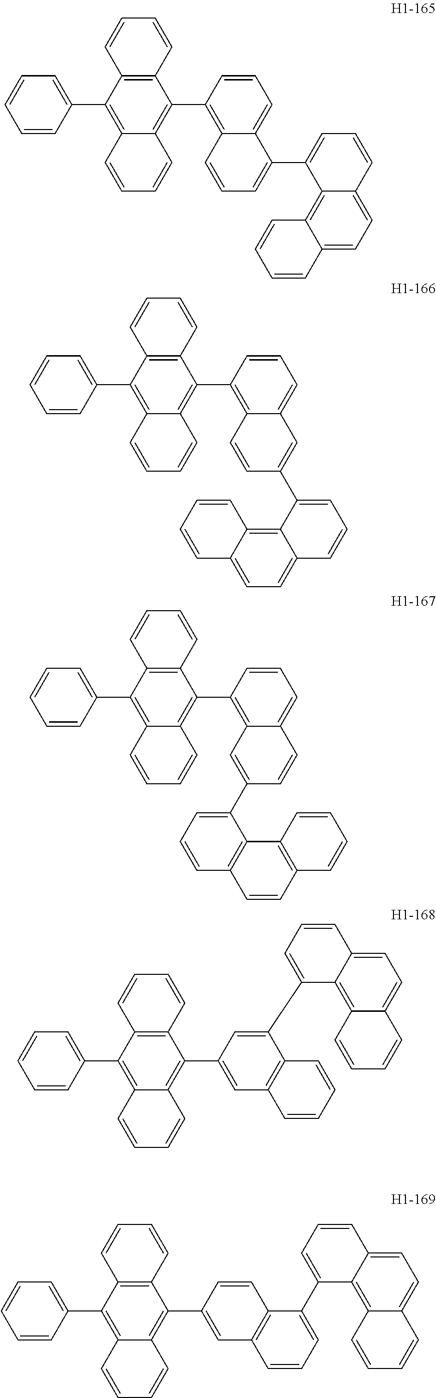 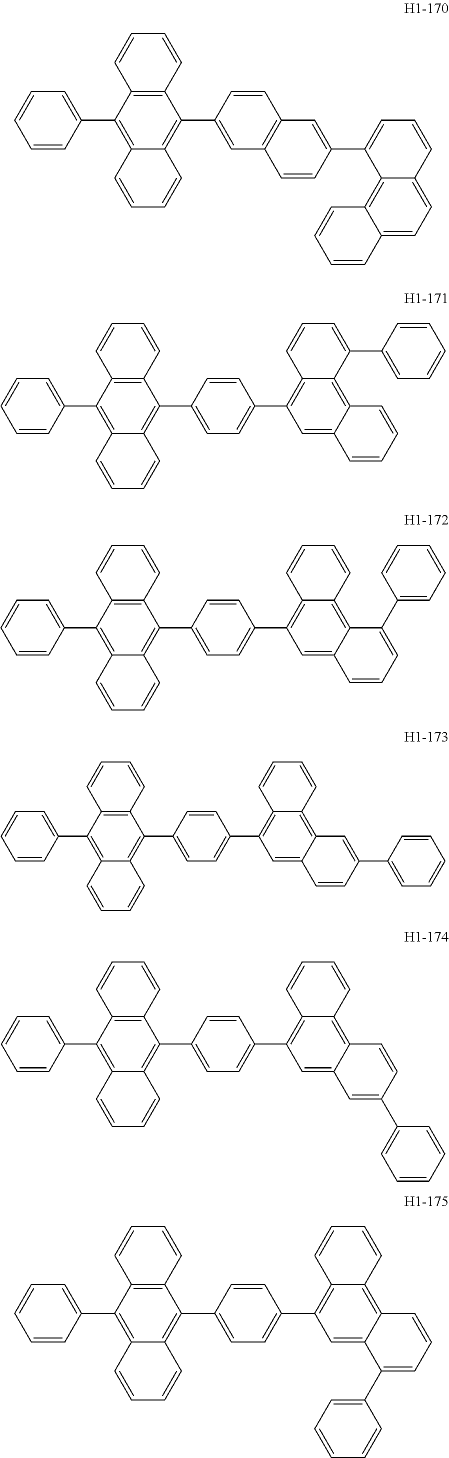 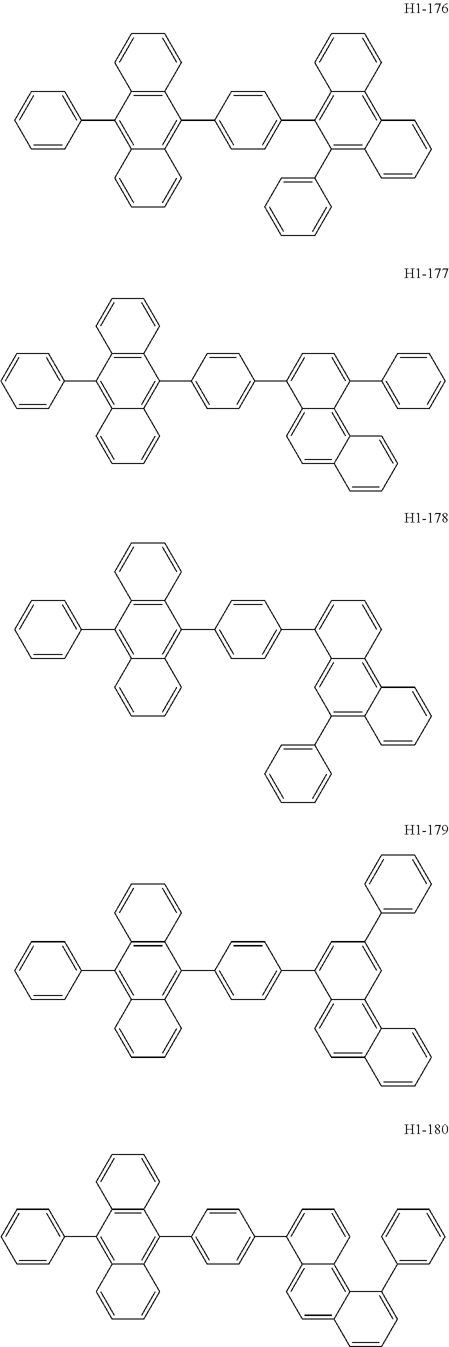 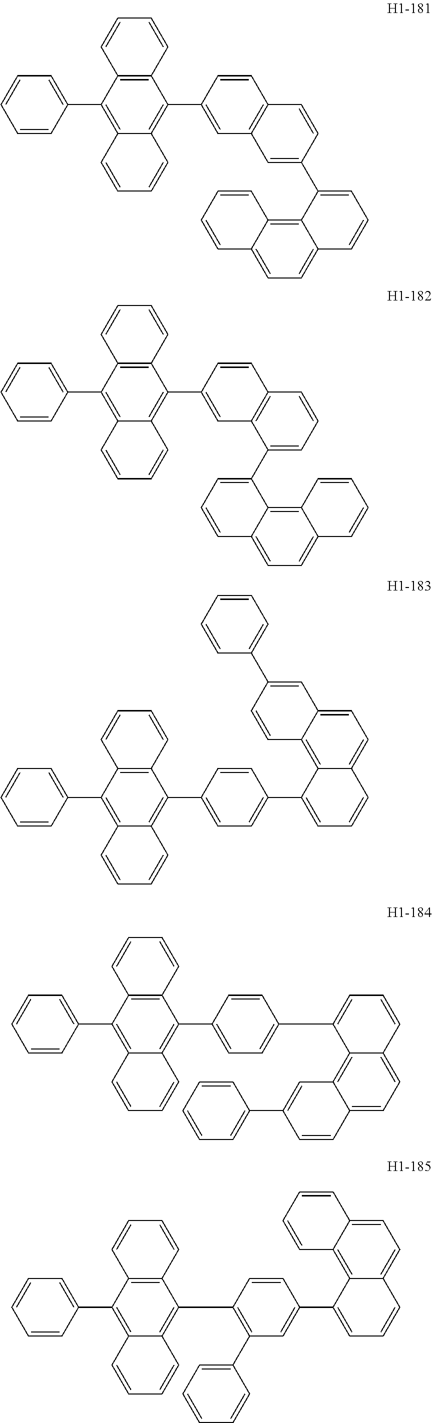 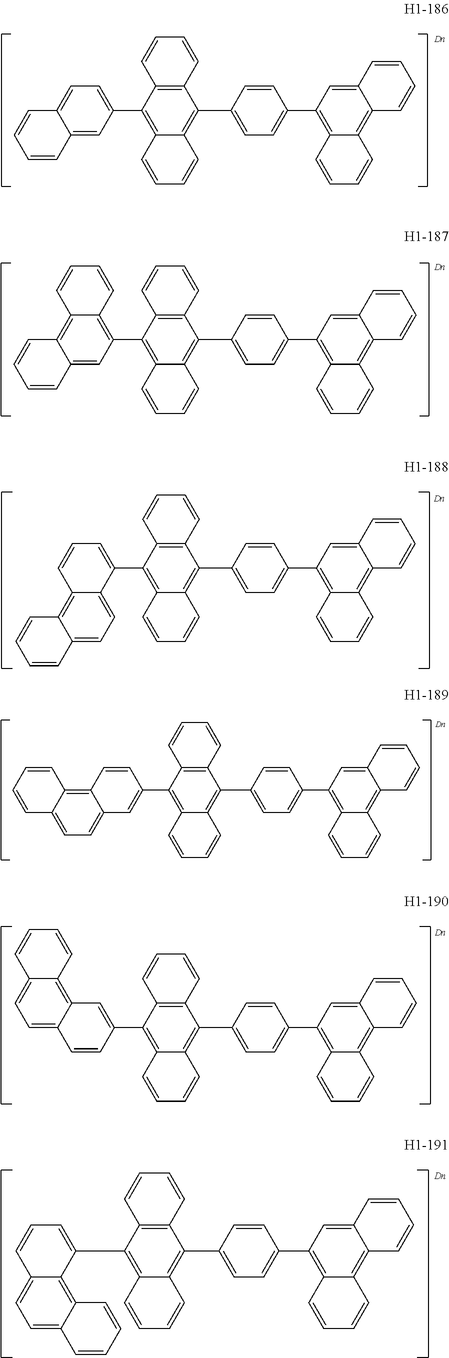 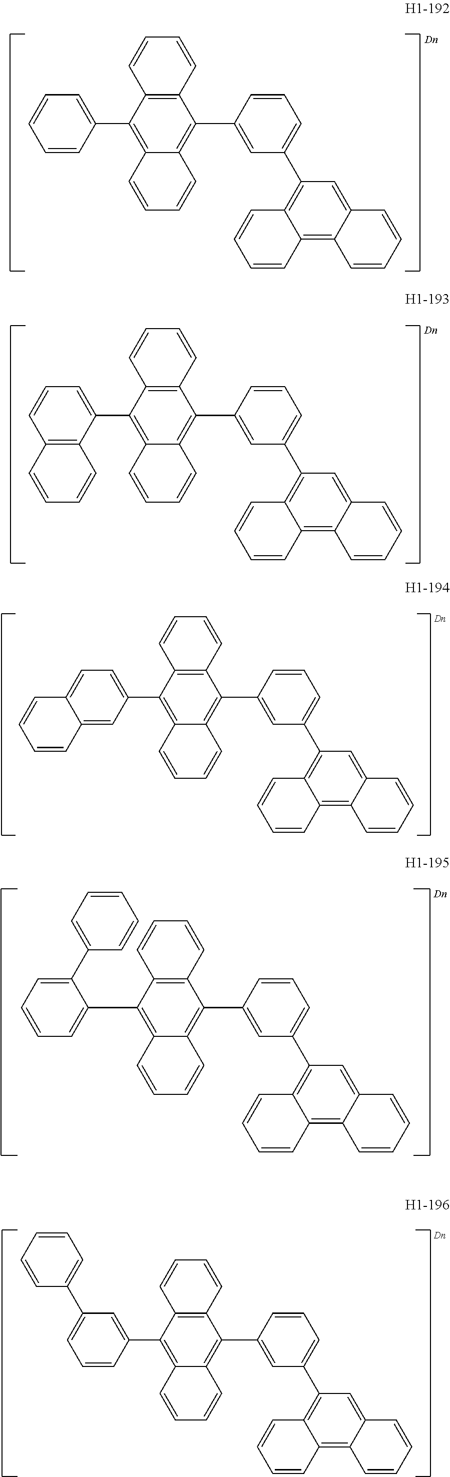 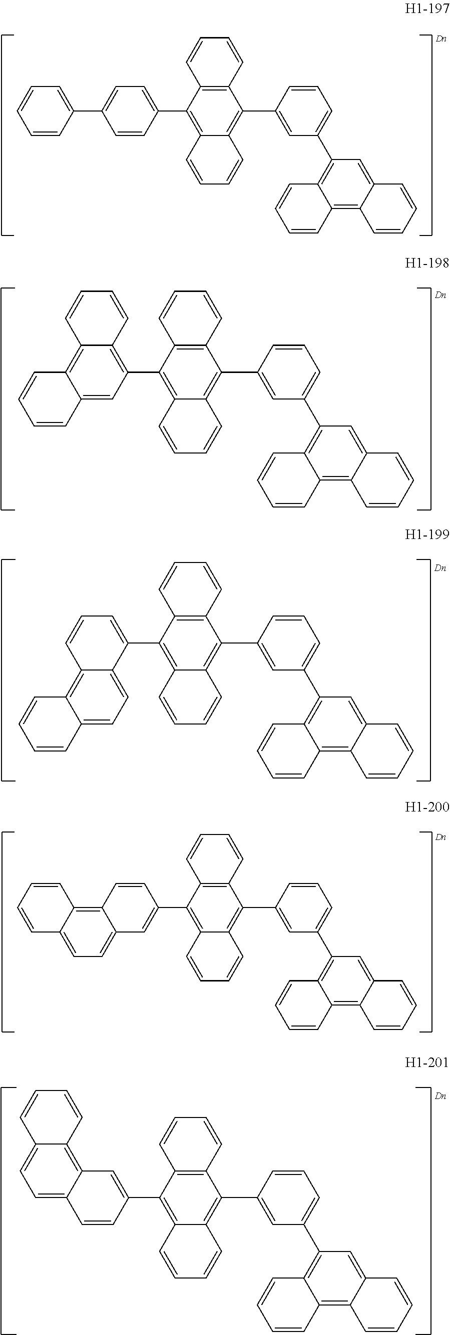 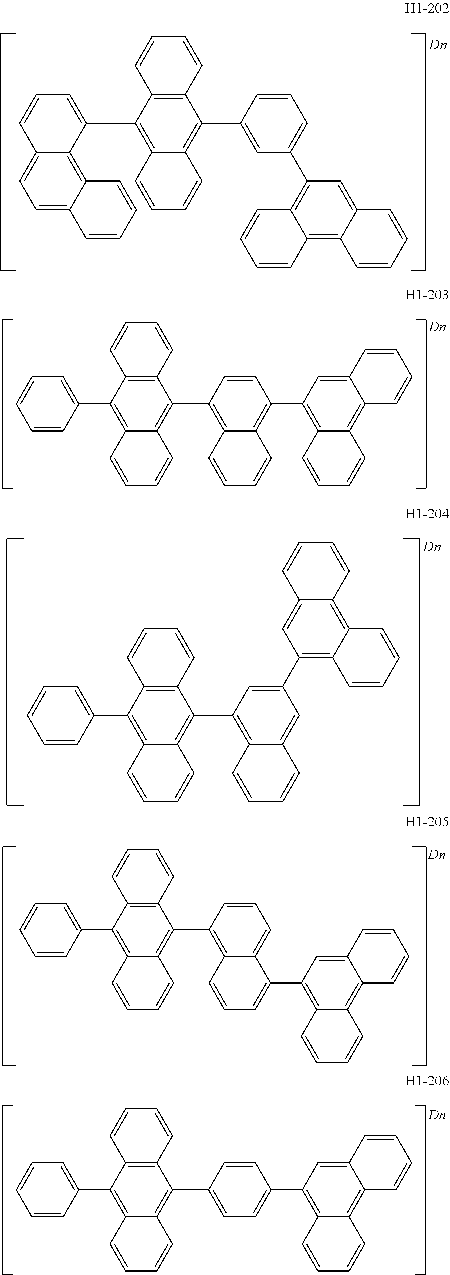 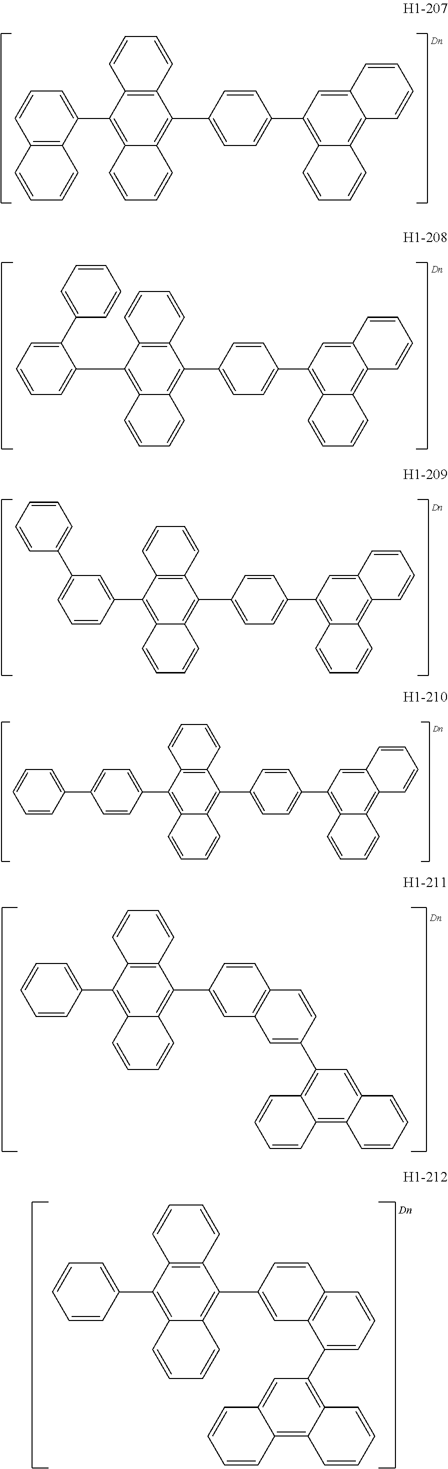 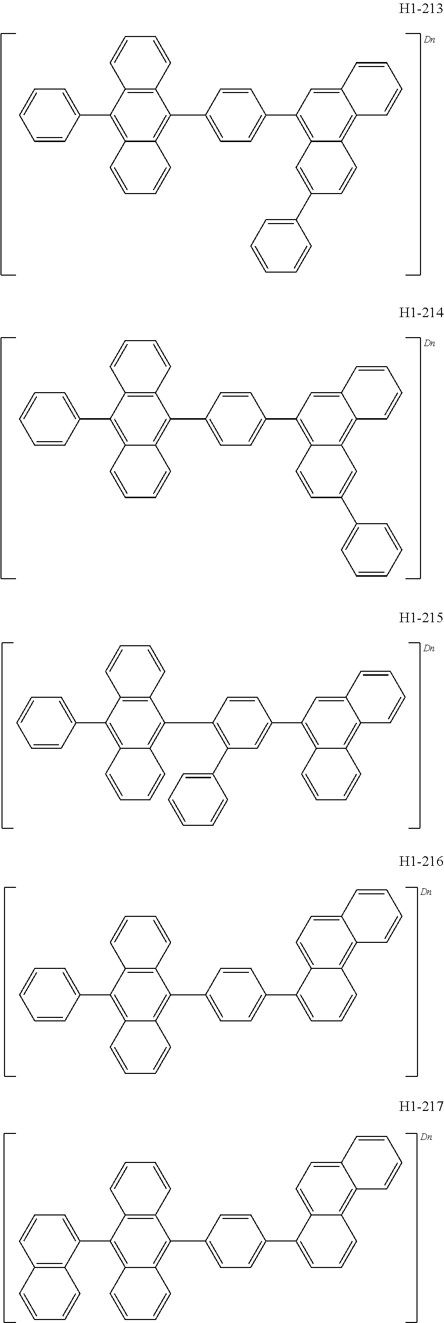 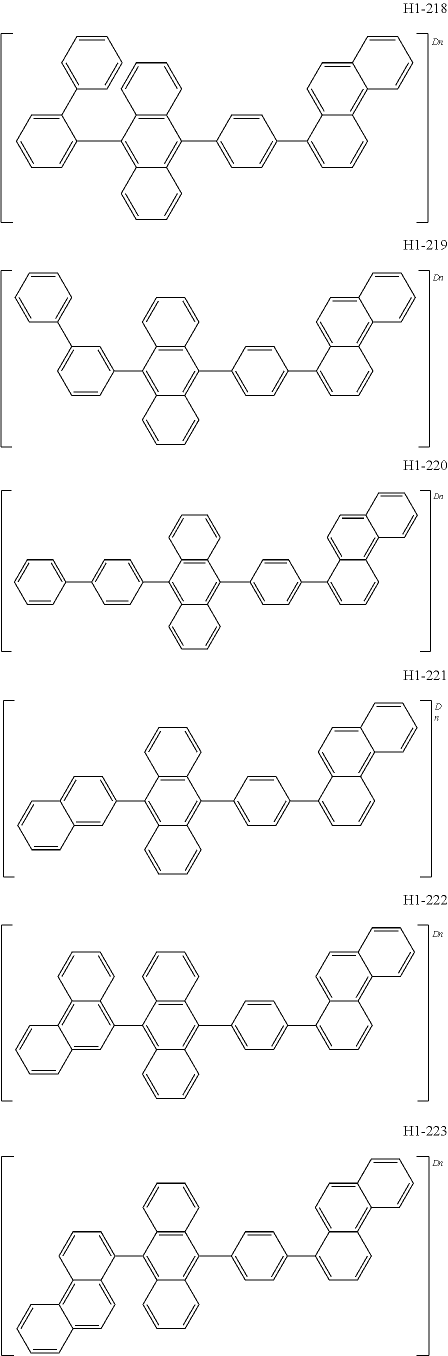 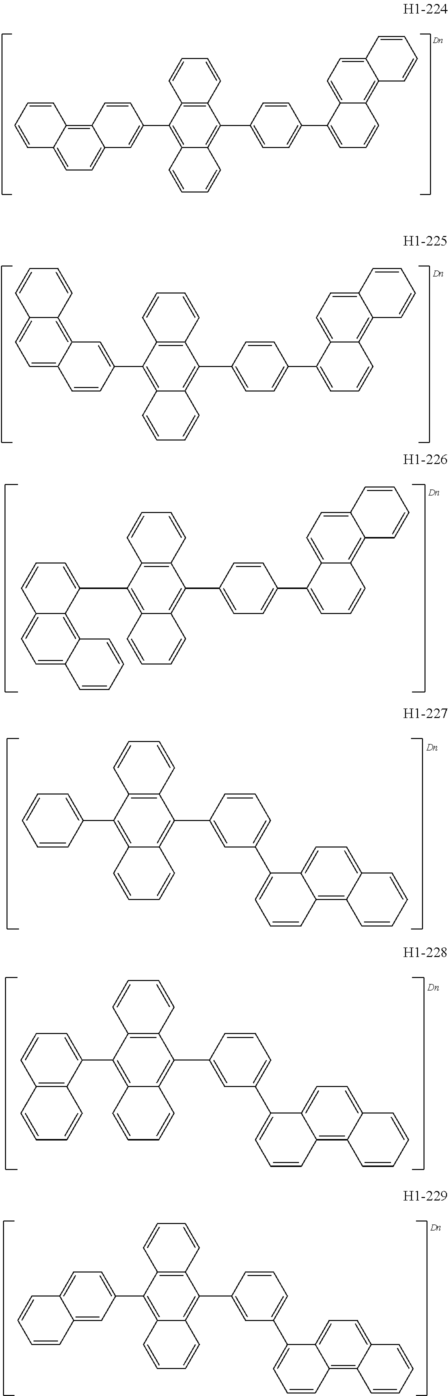 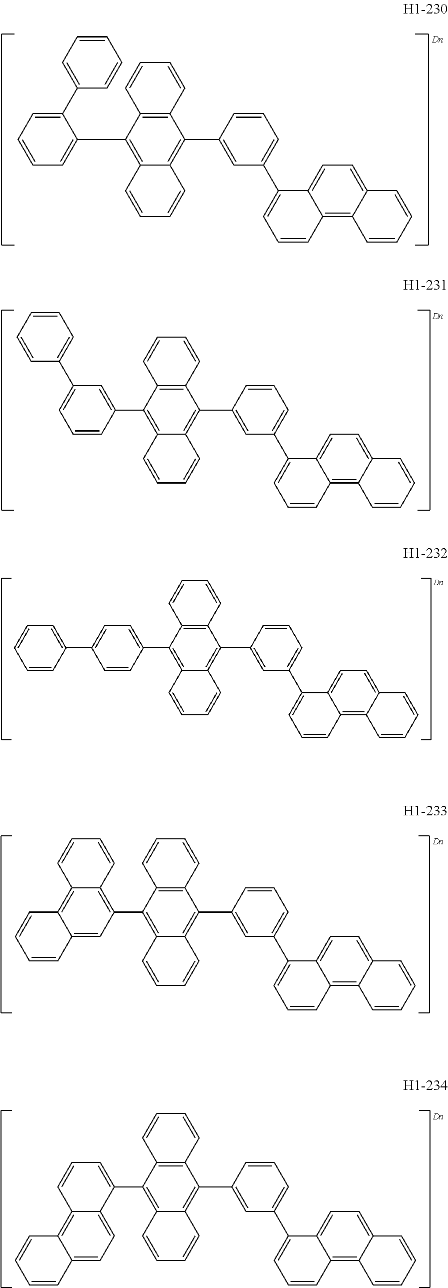 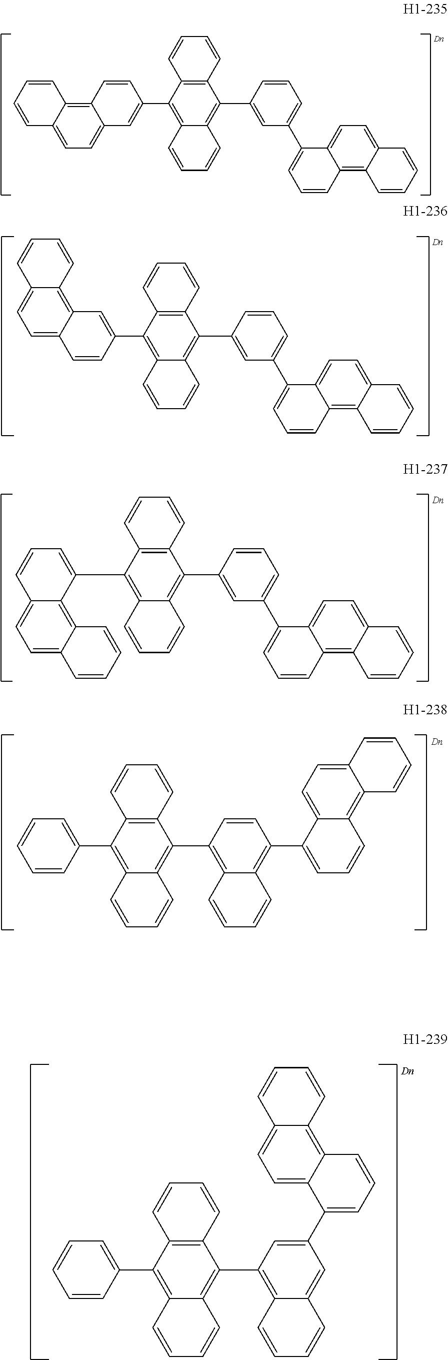 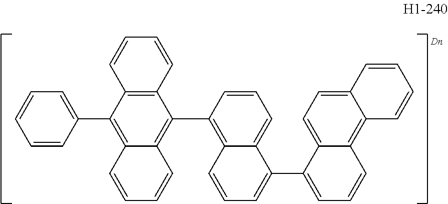 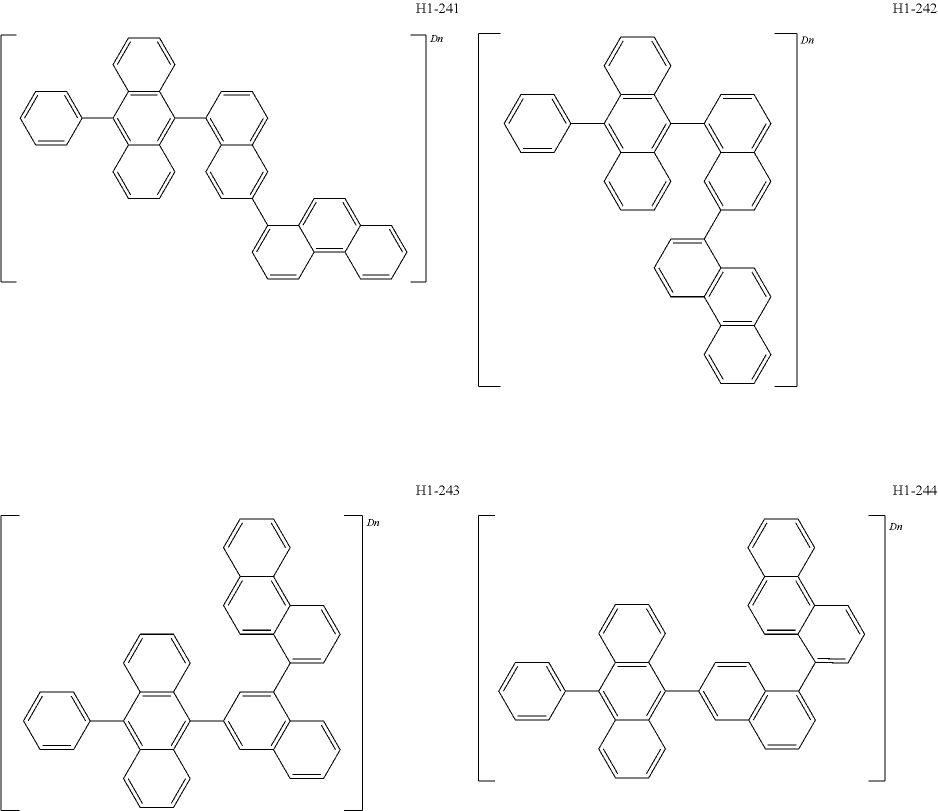 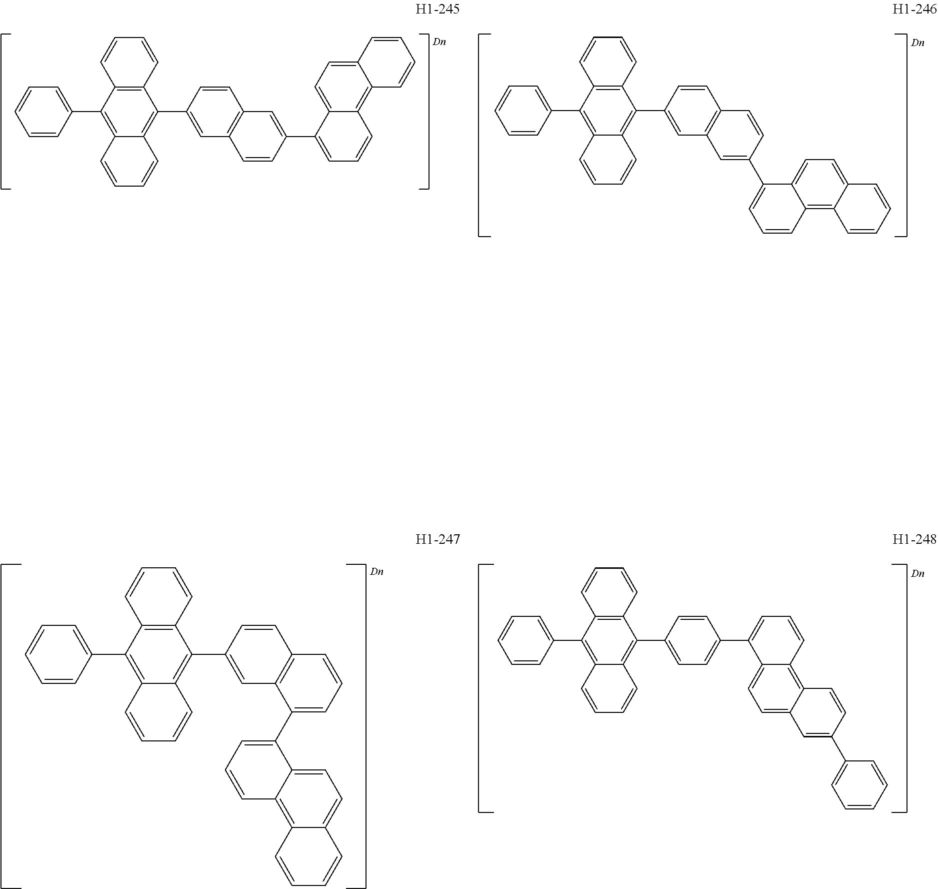 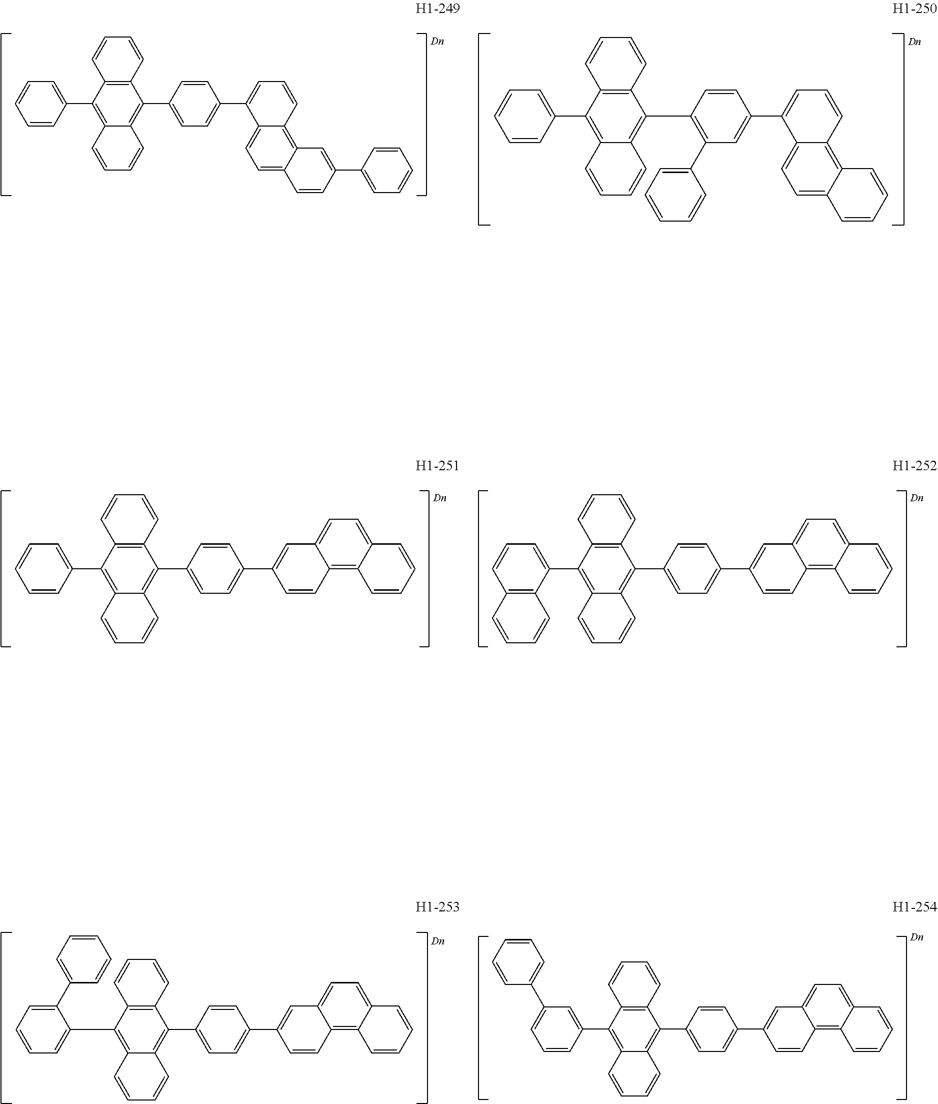 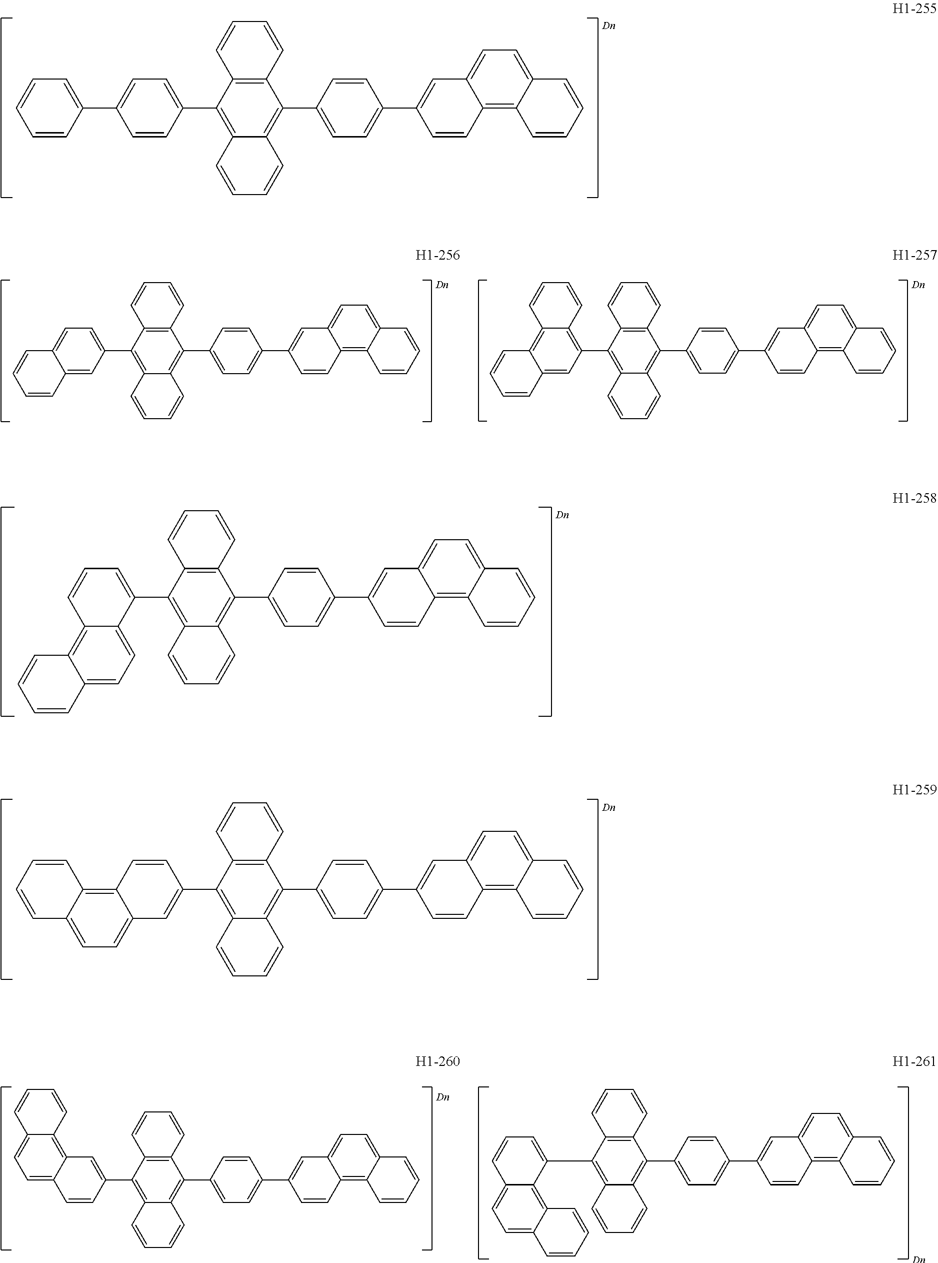 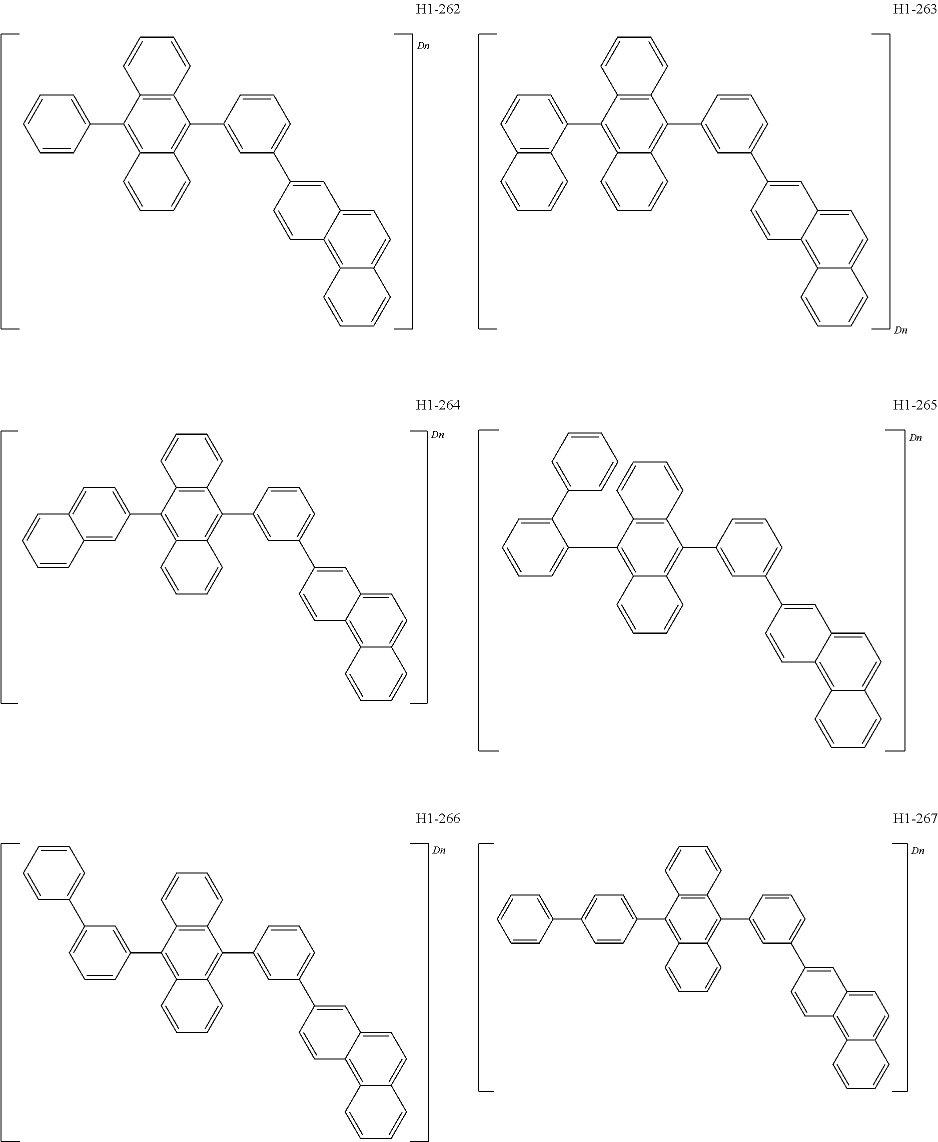 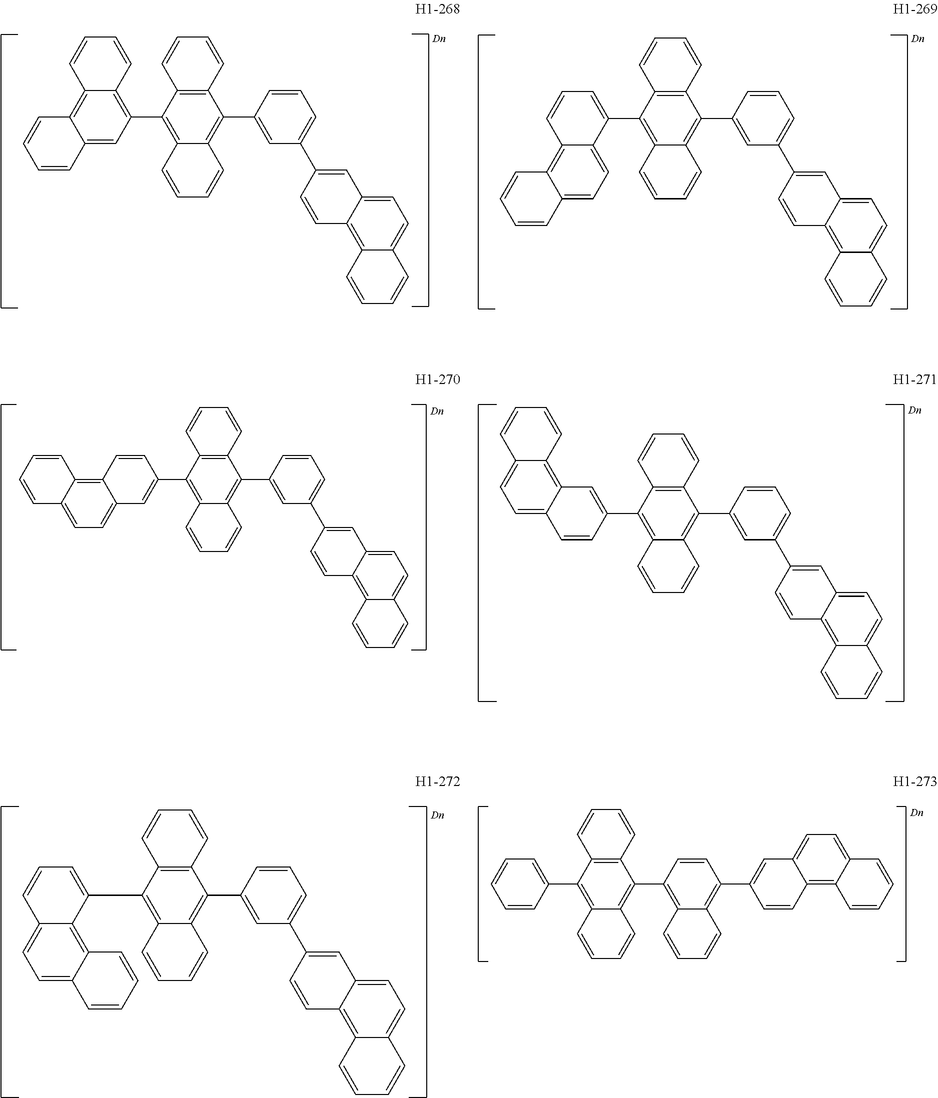 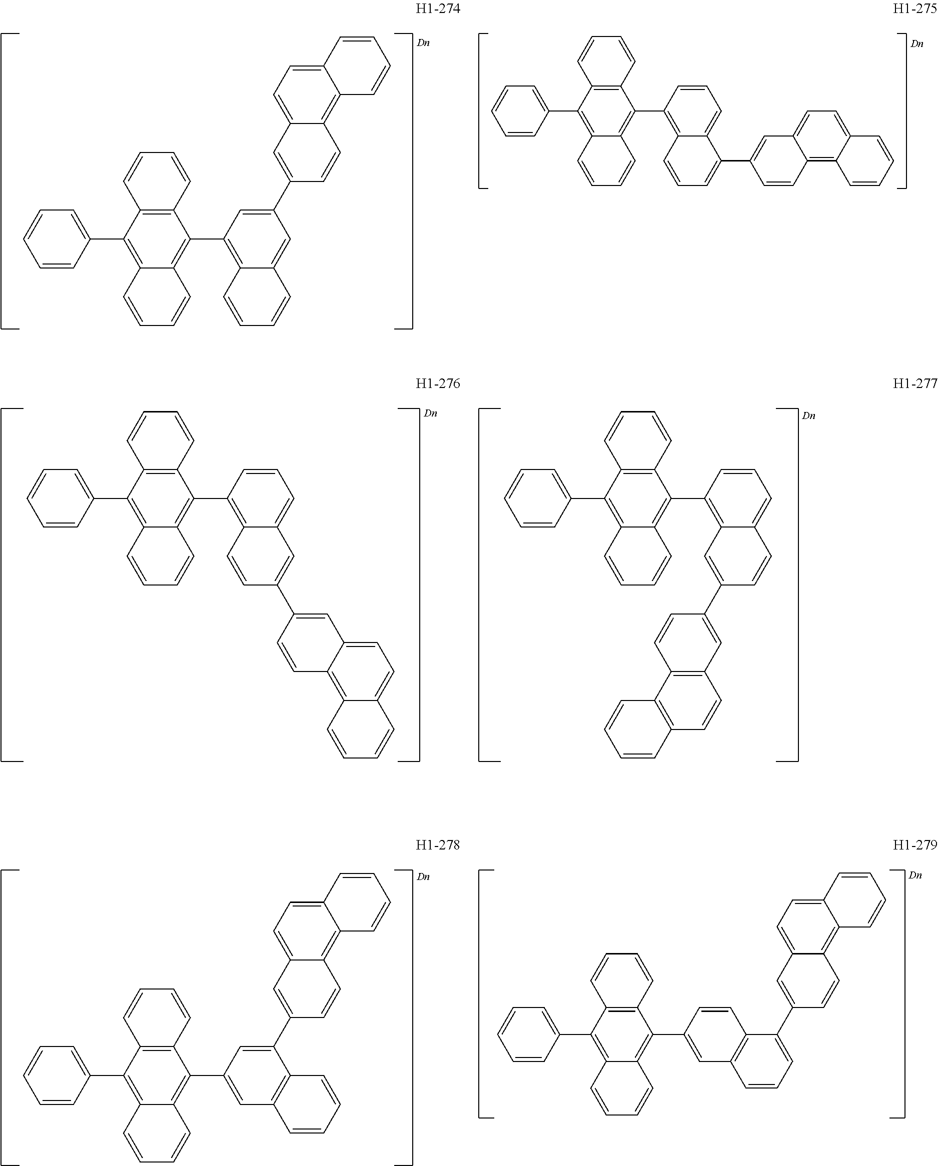 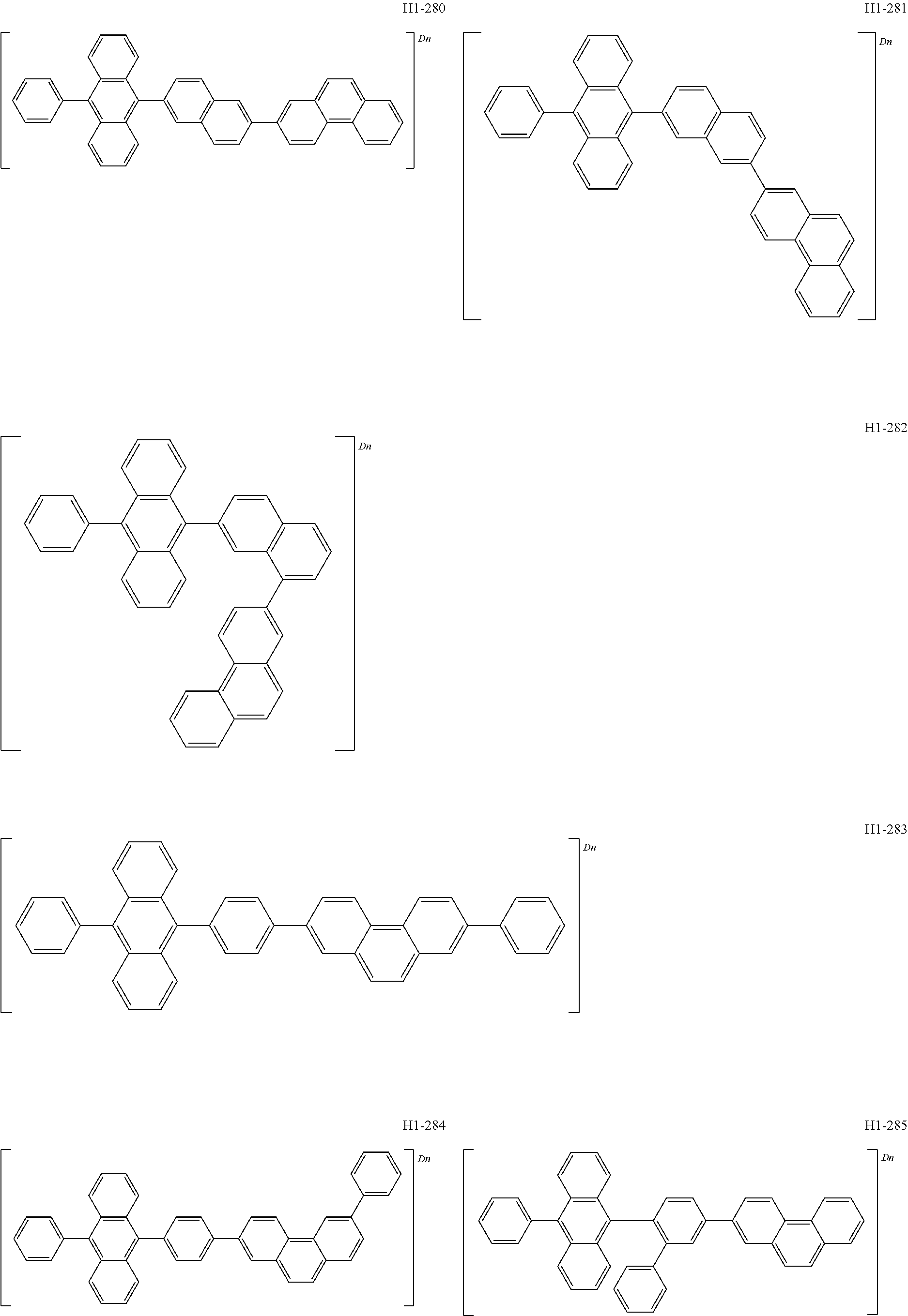 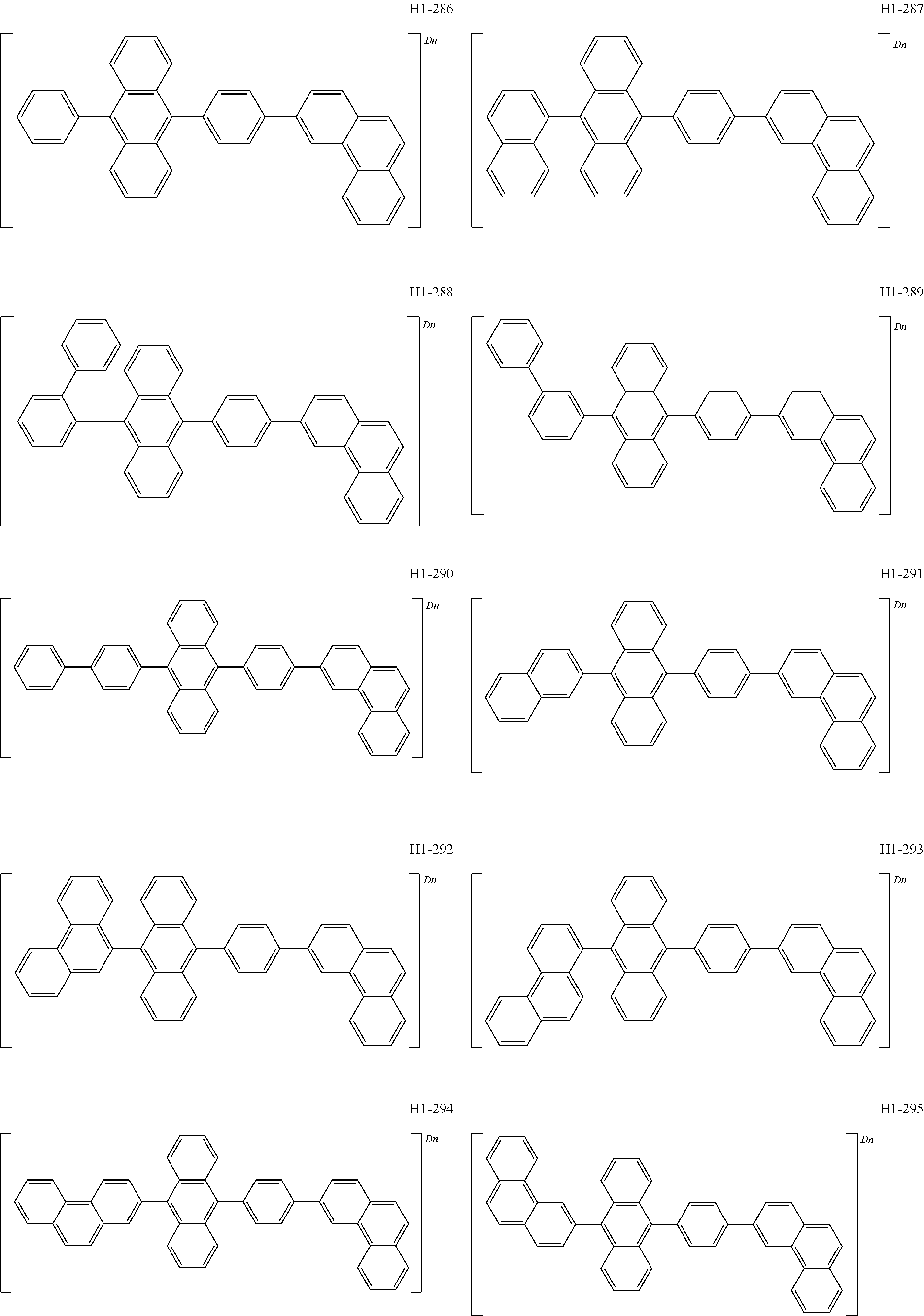 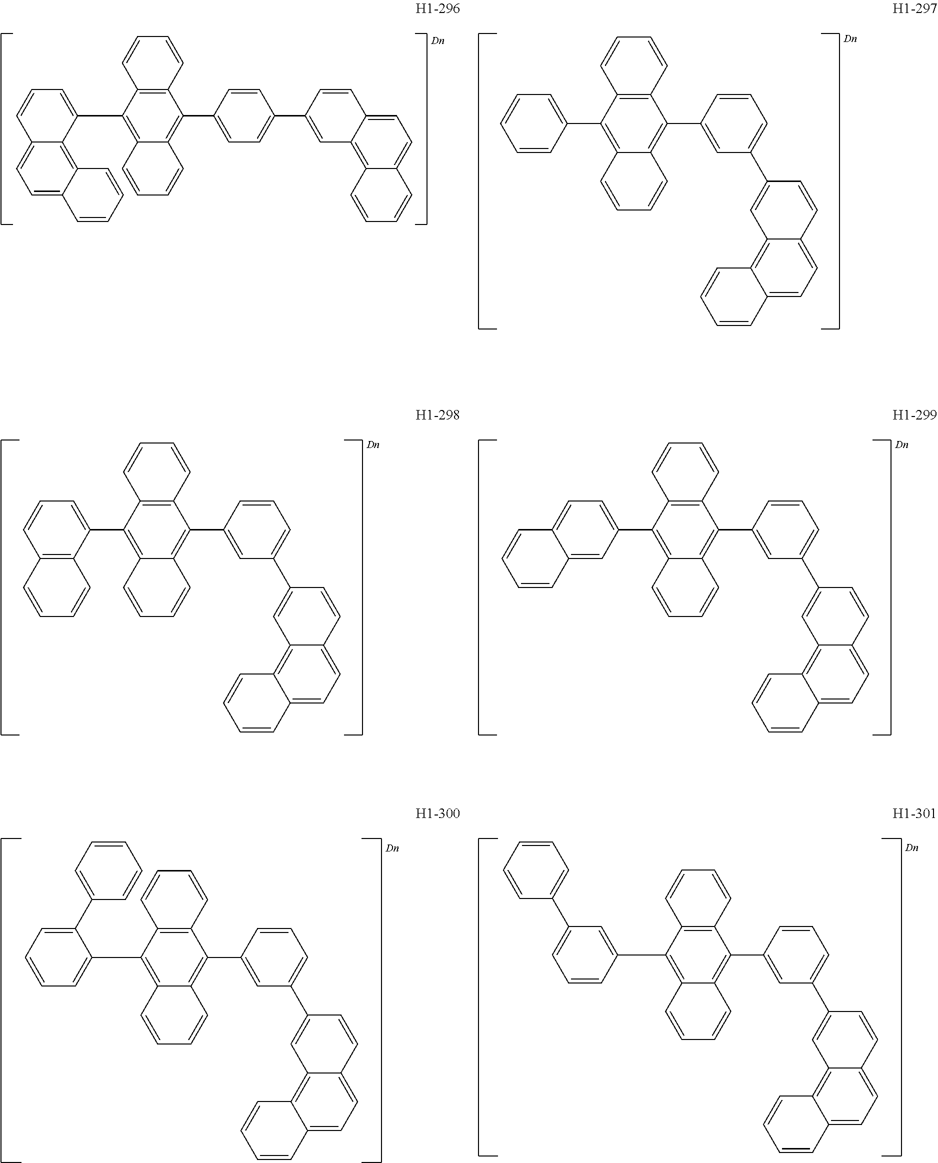 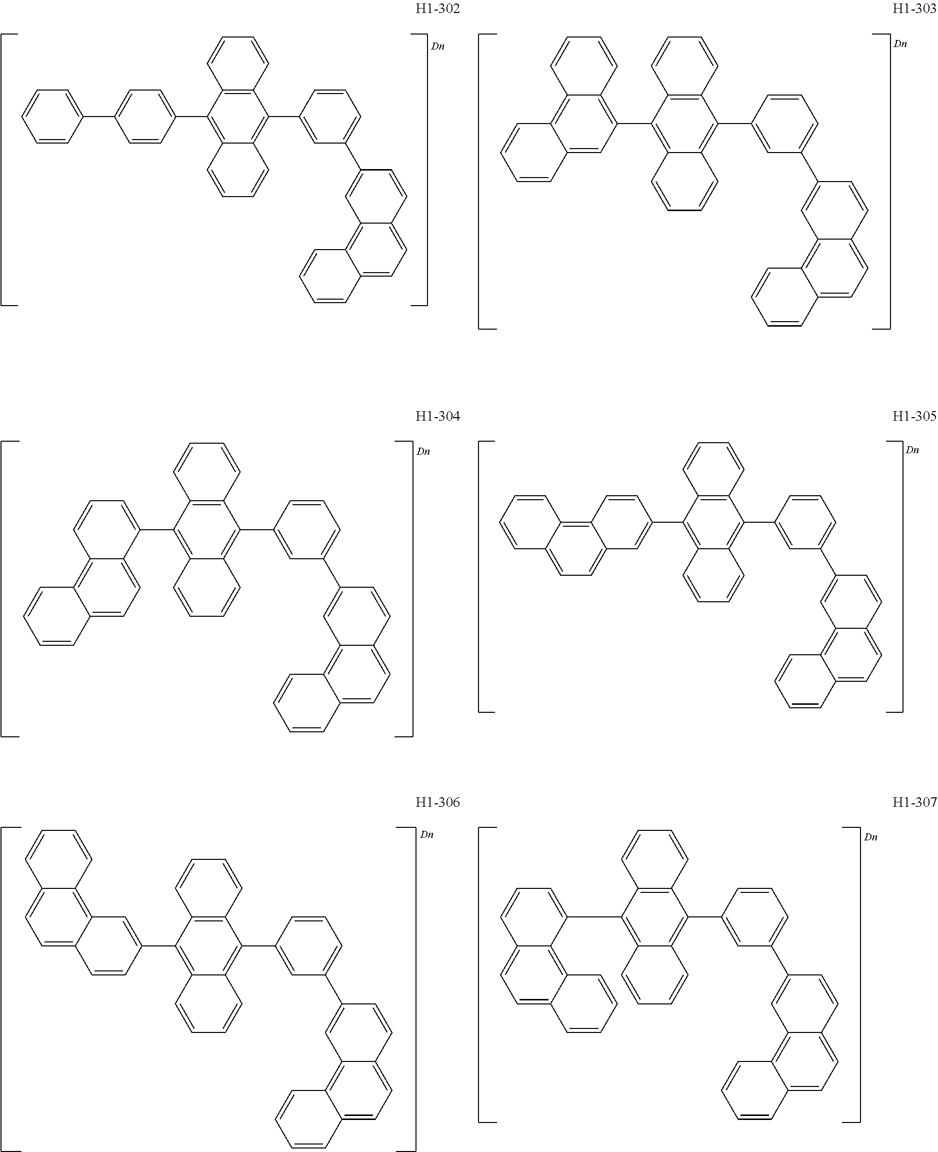 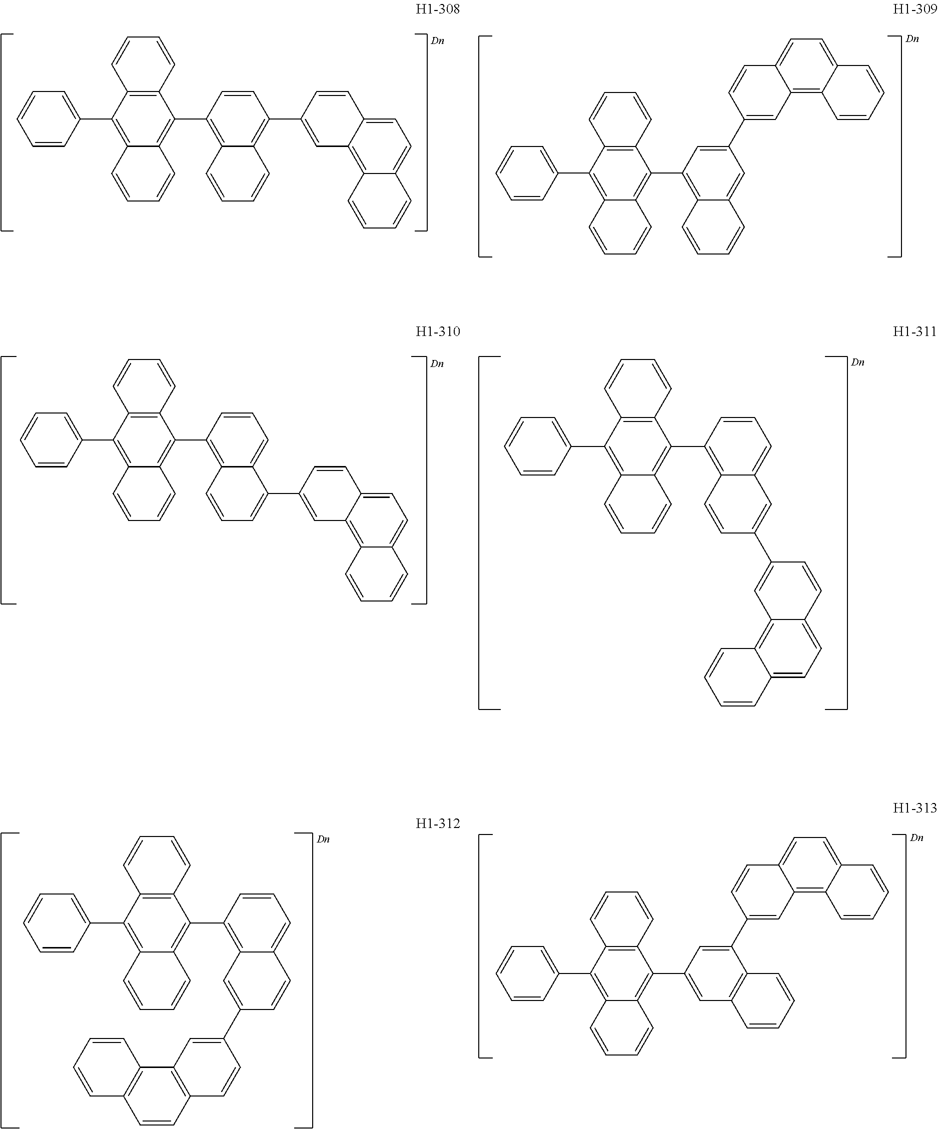 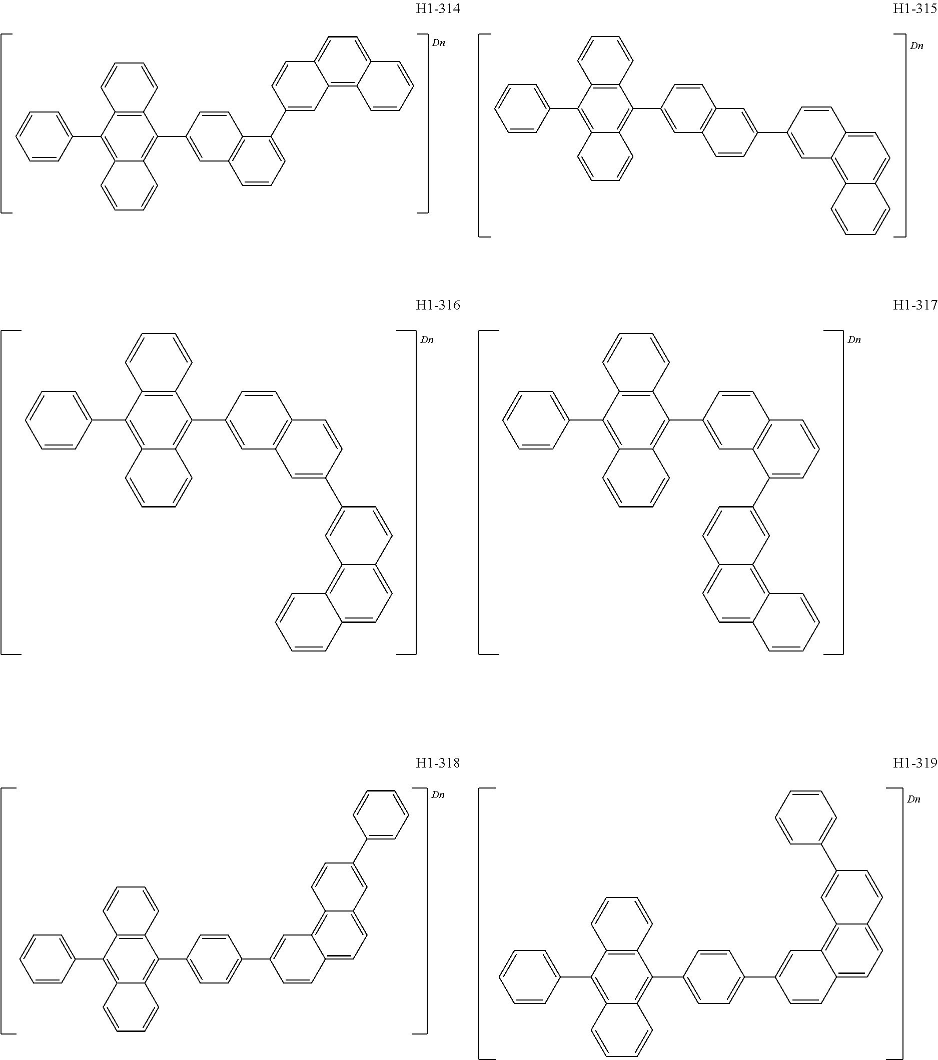 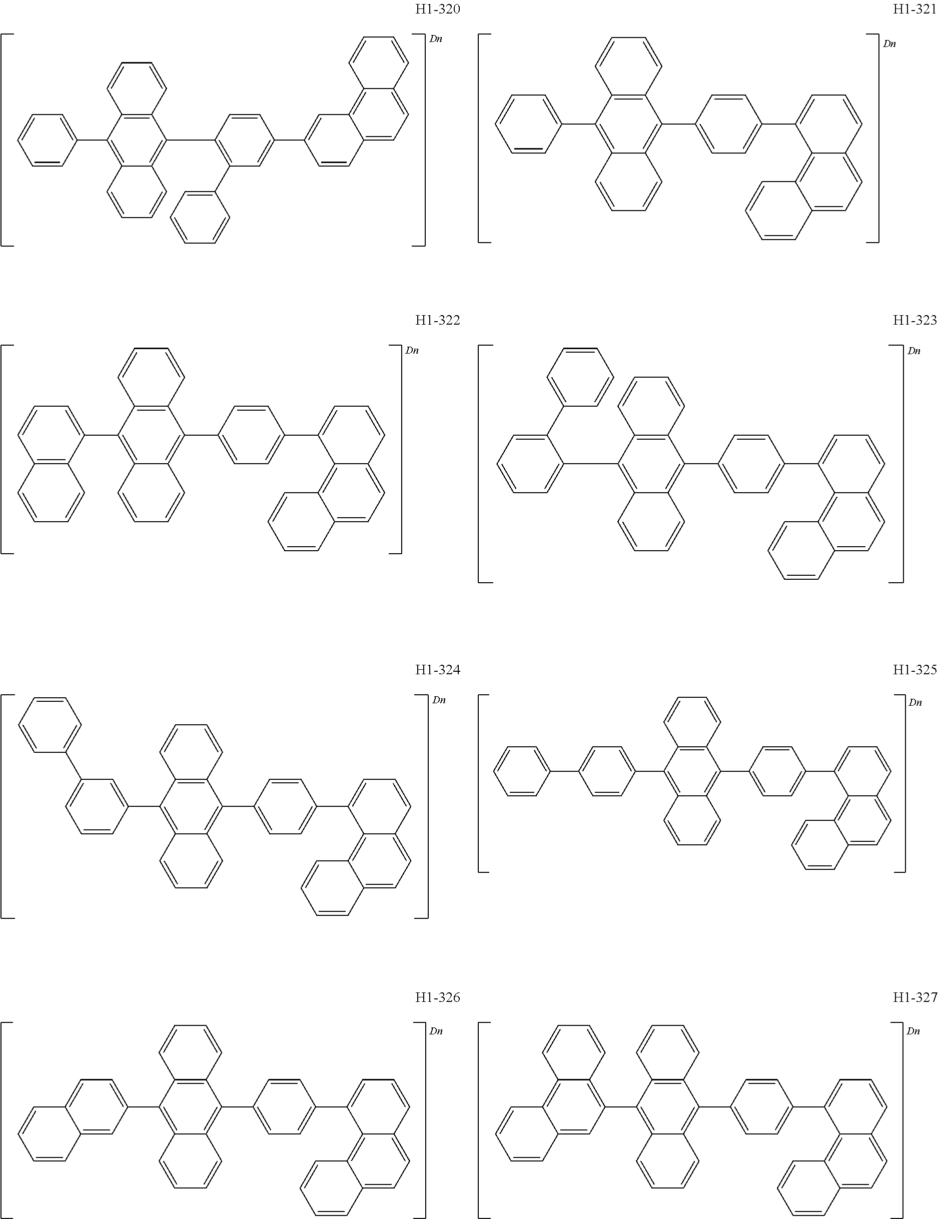 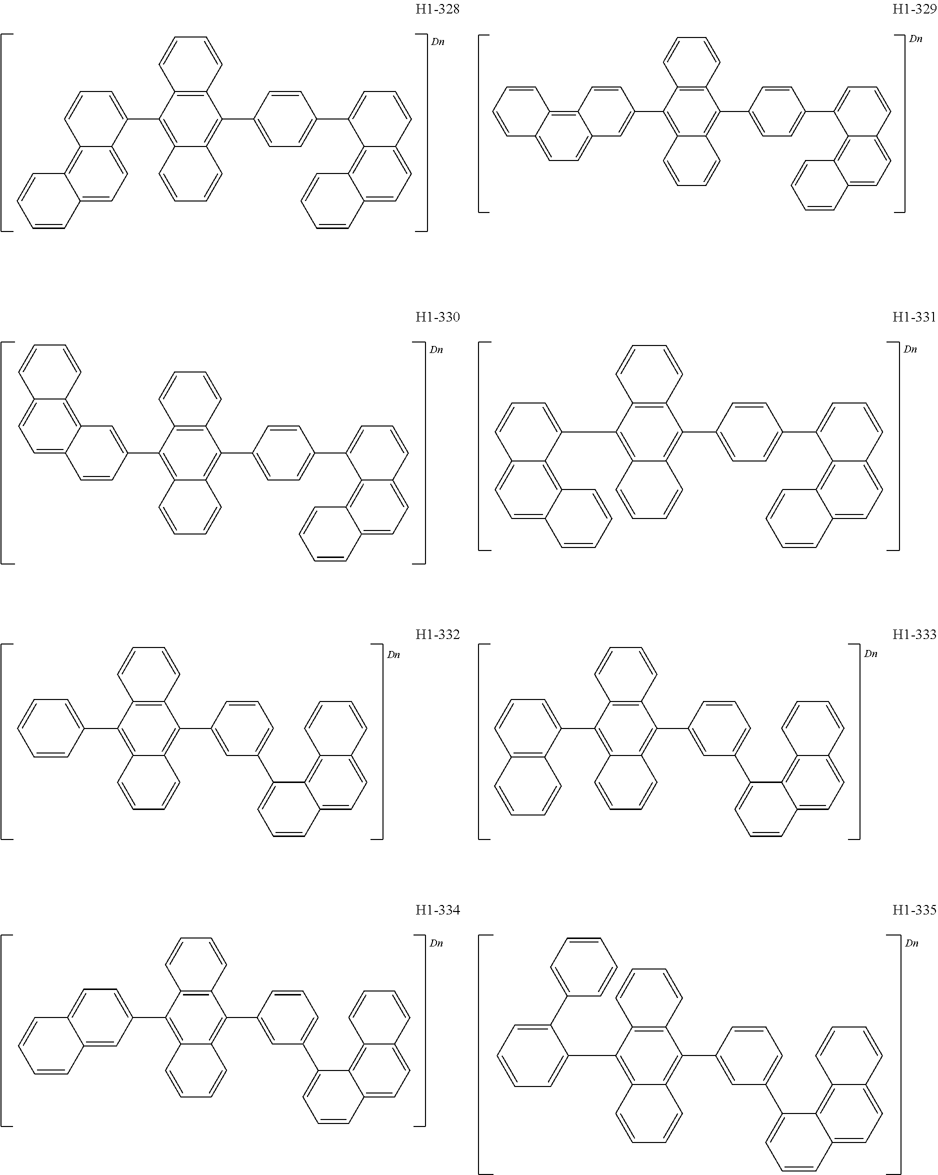 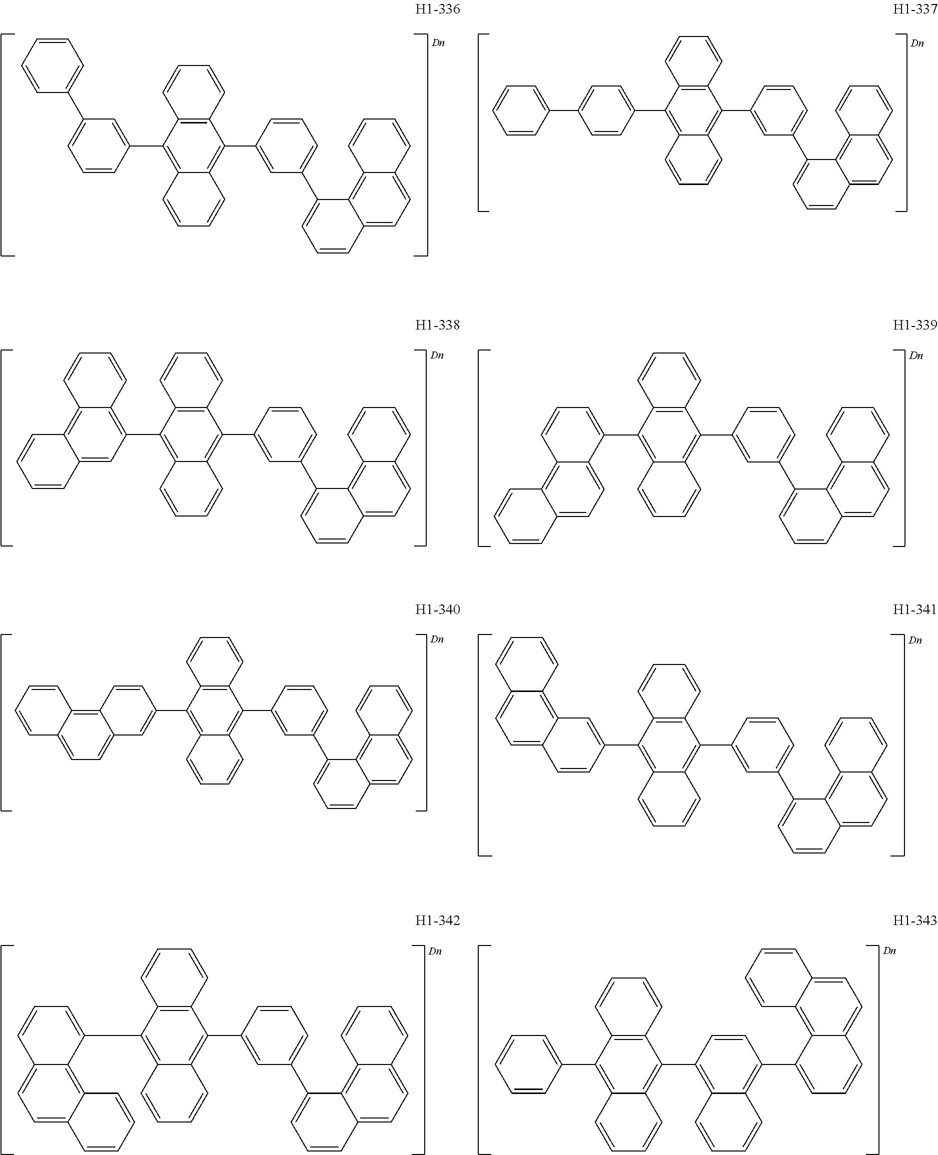 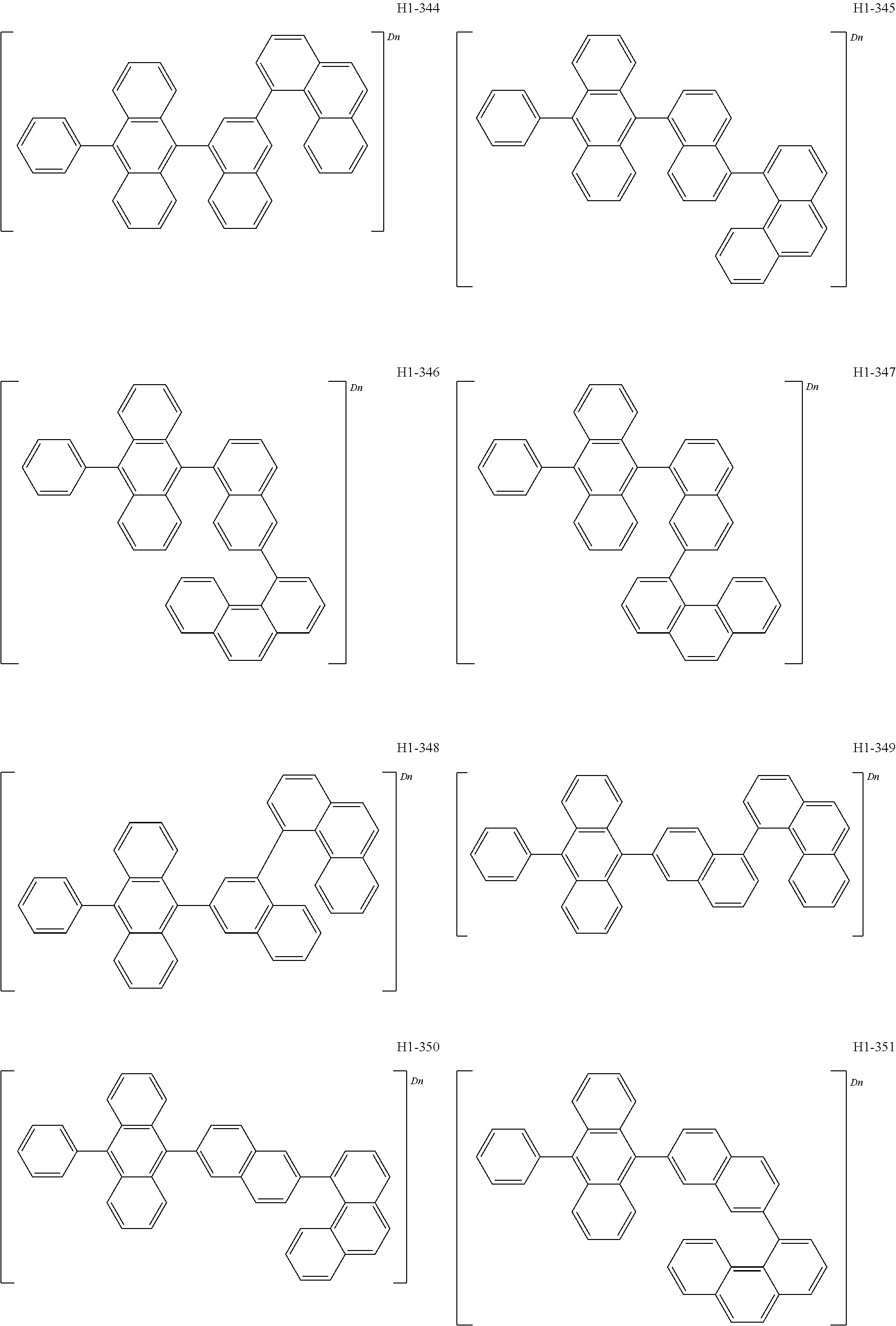 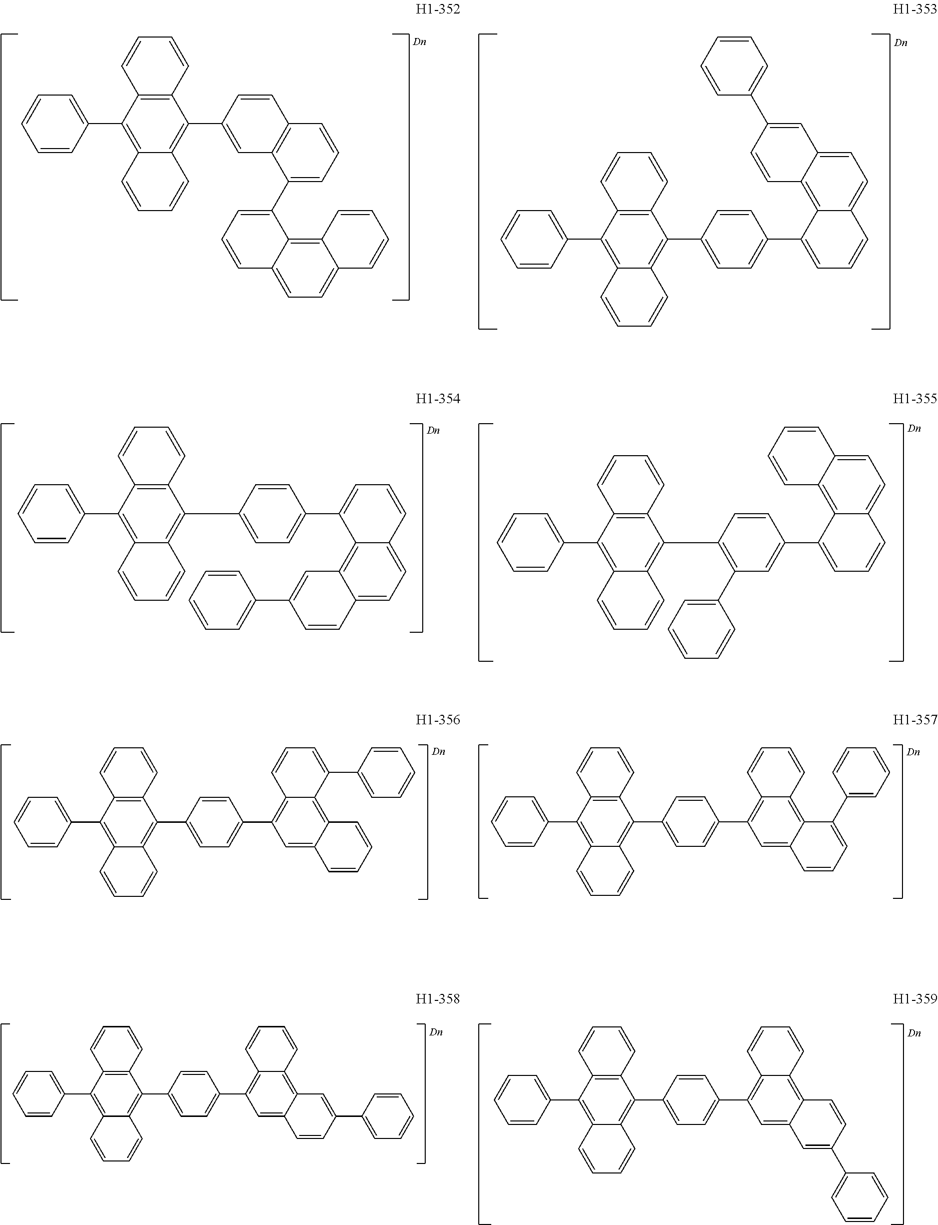 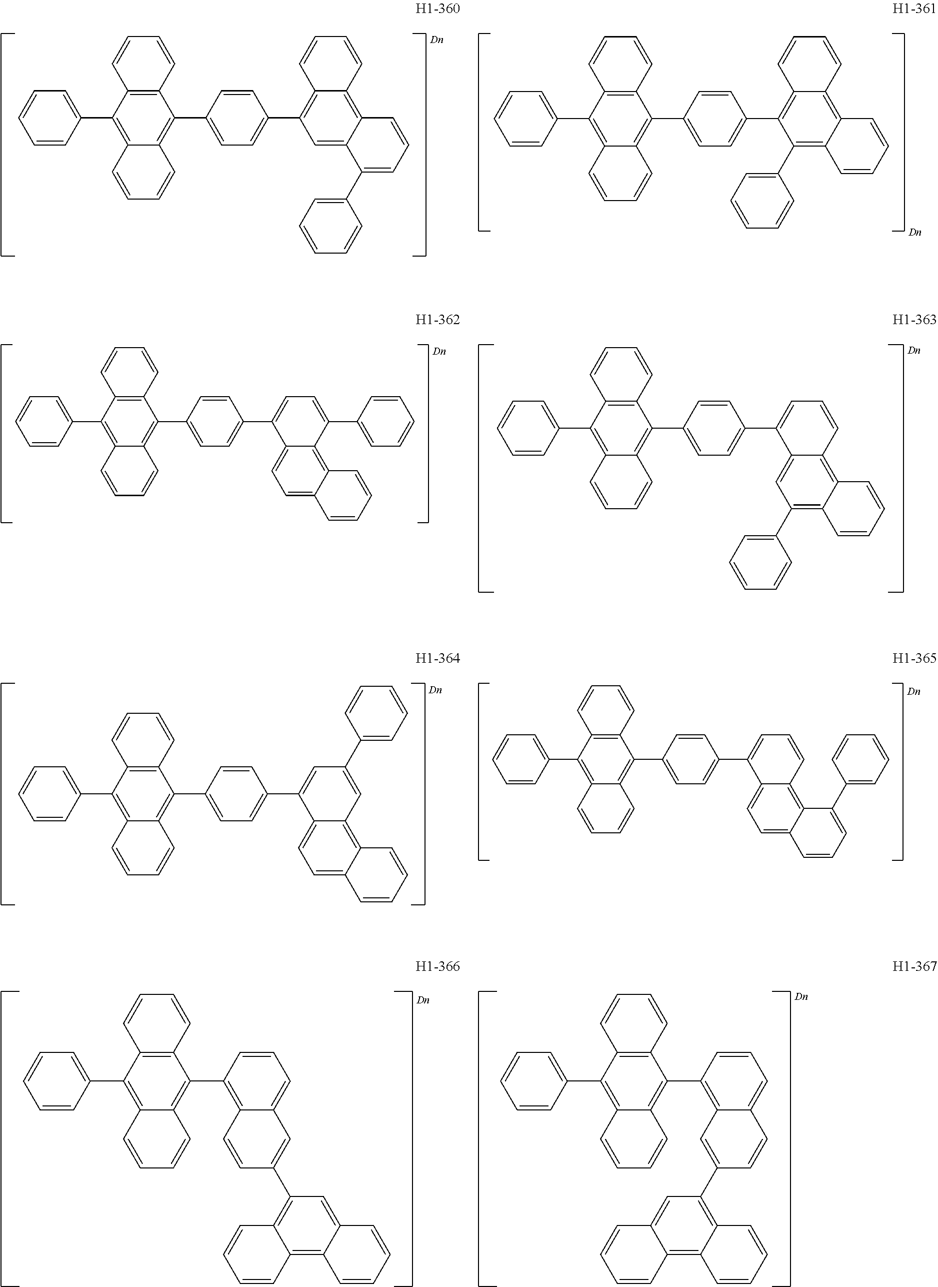 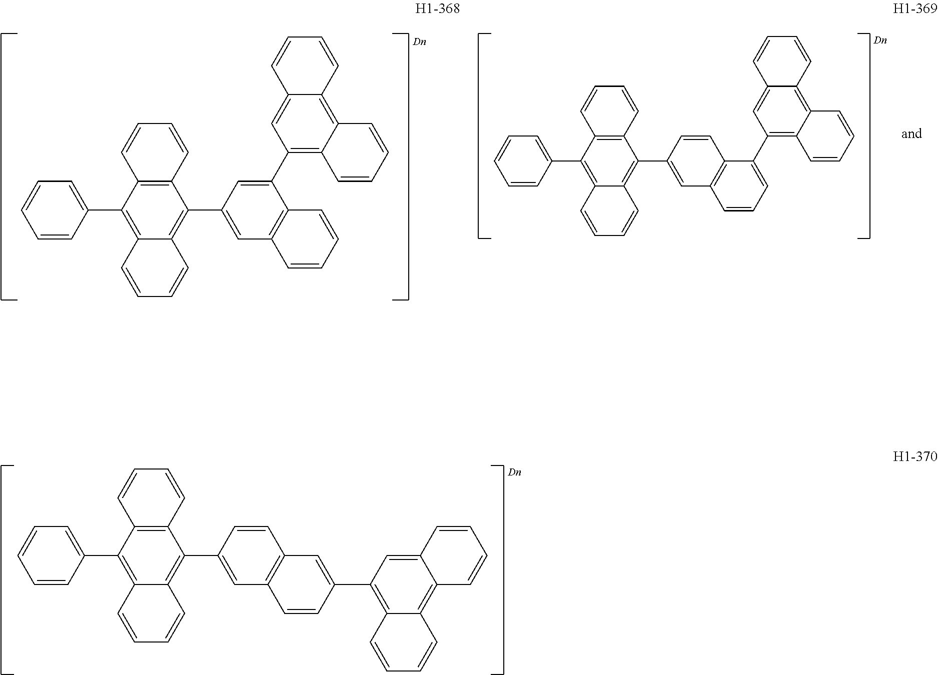
wherein Dₙ means that n hydrogens are substituted with deuterium, and n is an integer from 1 to the maximum number of hydrogens in the compound.

7. The plurality of host materials according to claim 1, wherein the compound represented by Formula 2 is at least one selected from the following compounds: 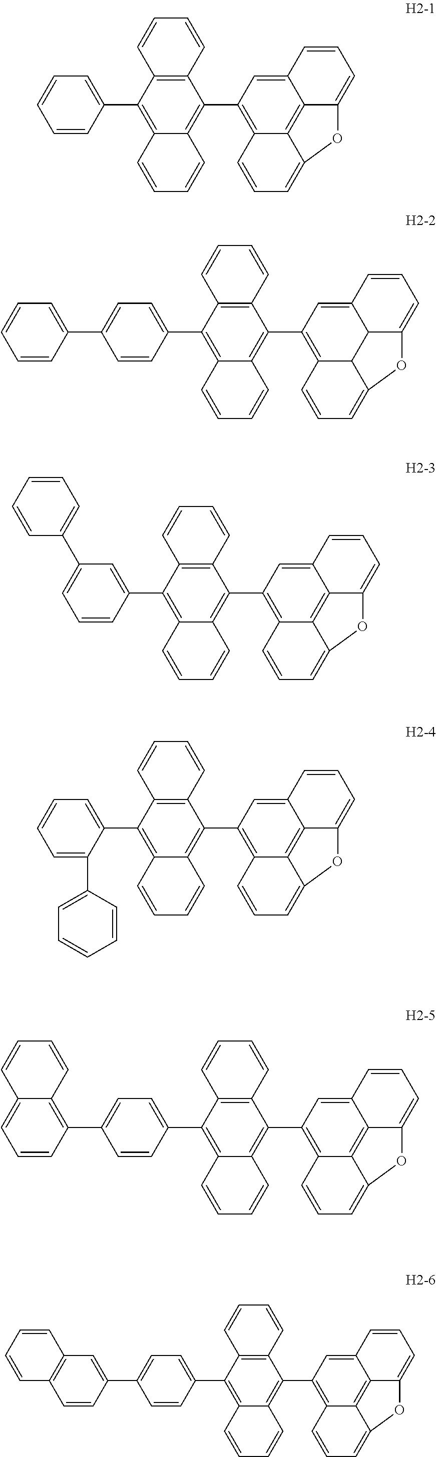 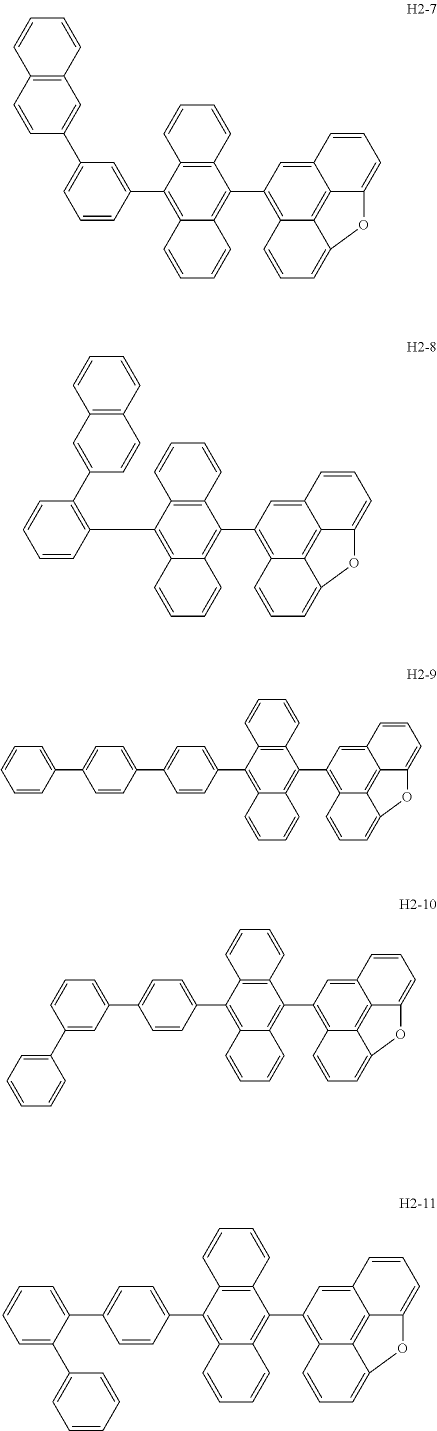 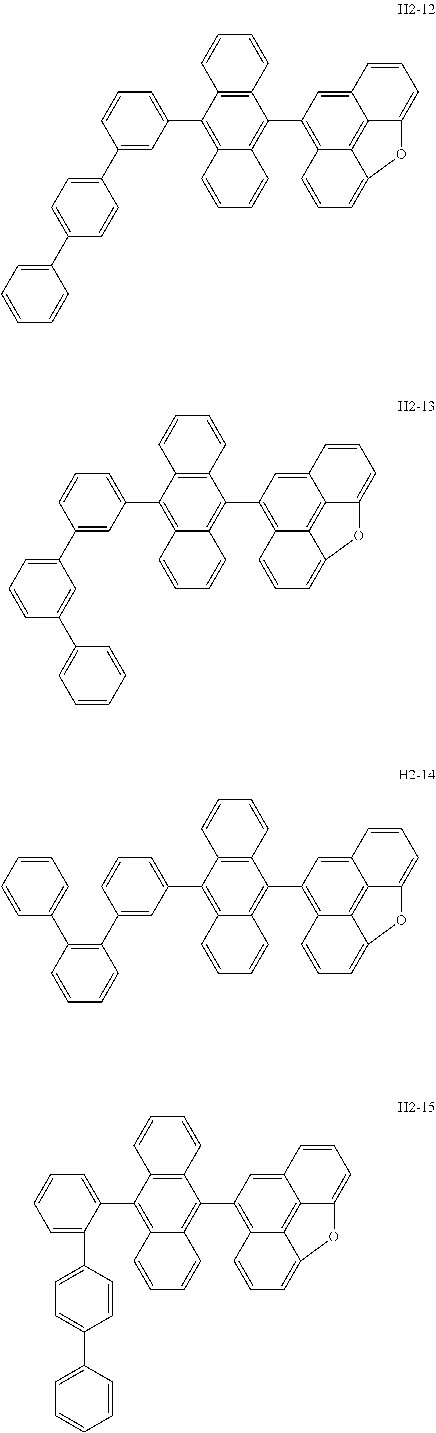 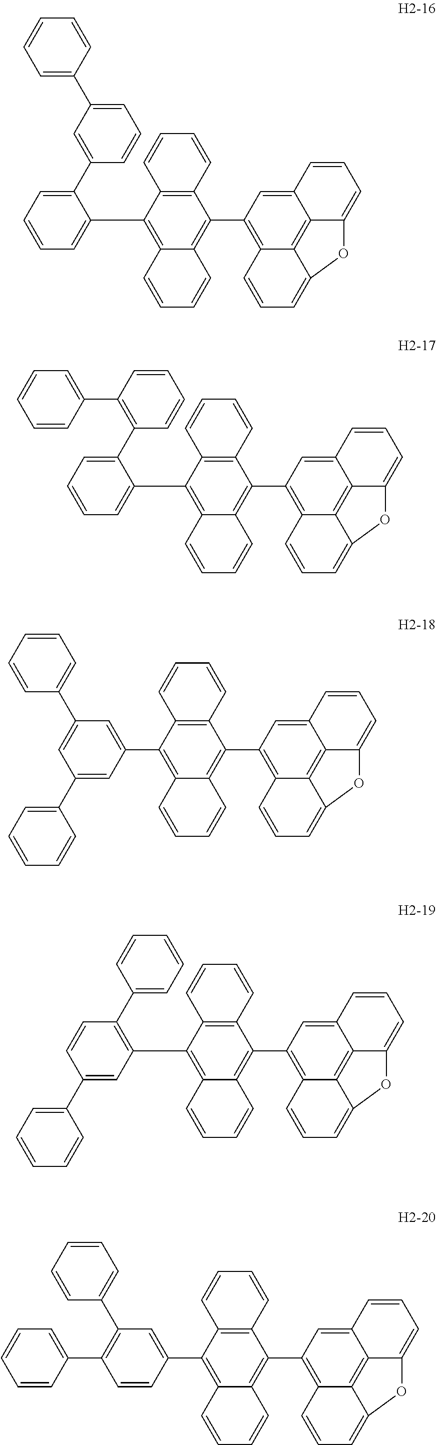 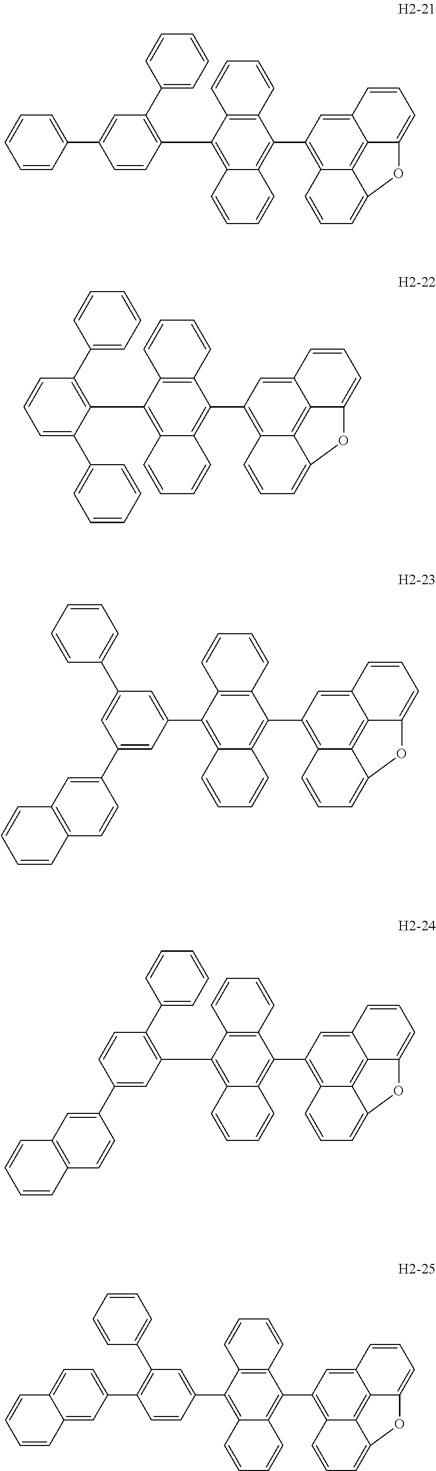 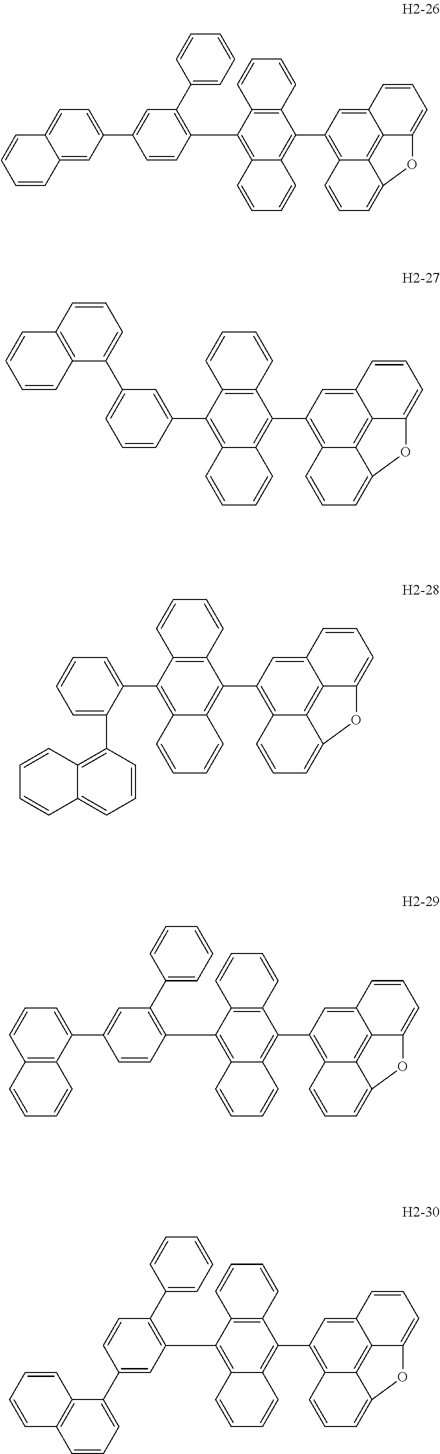 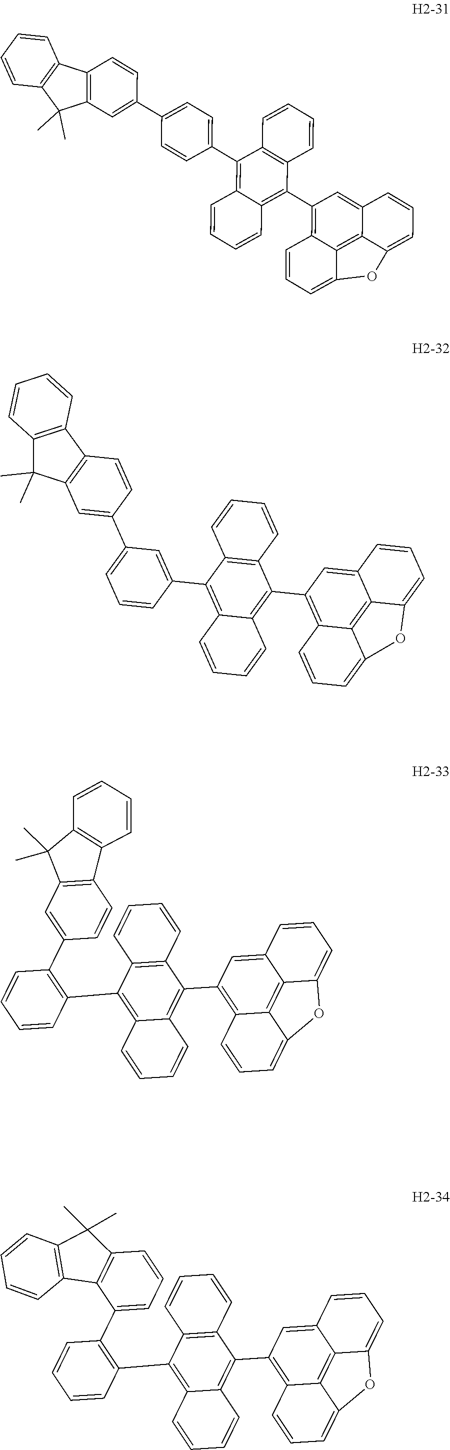 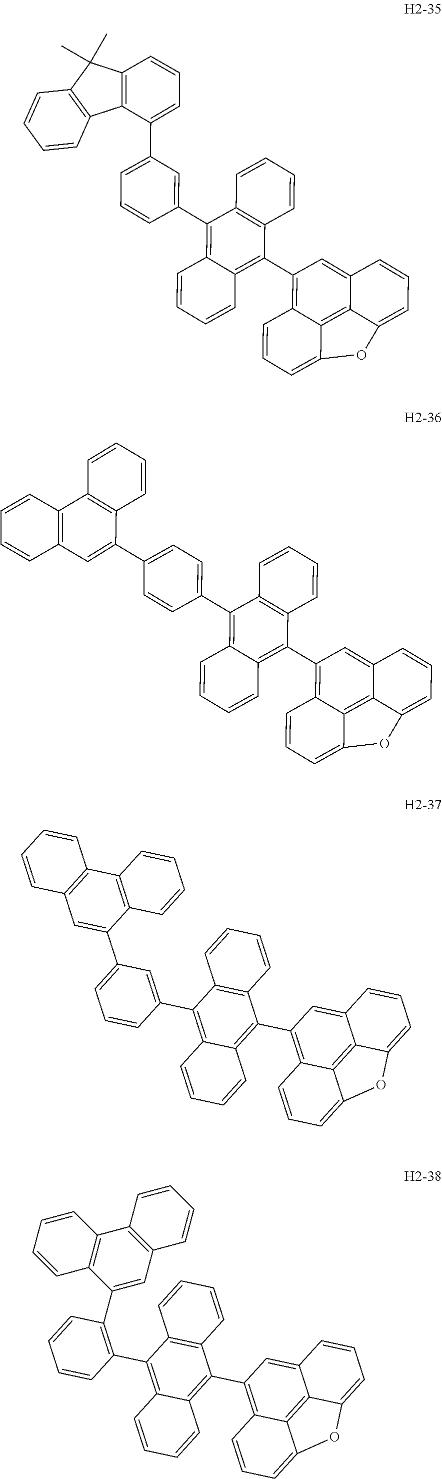 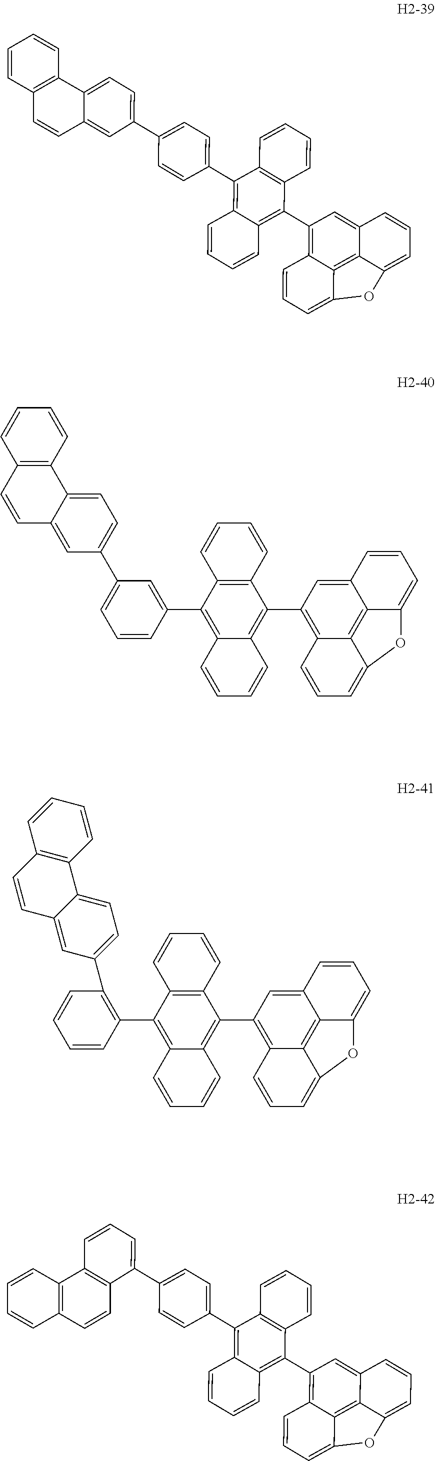 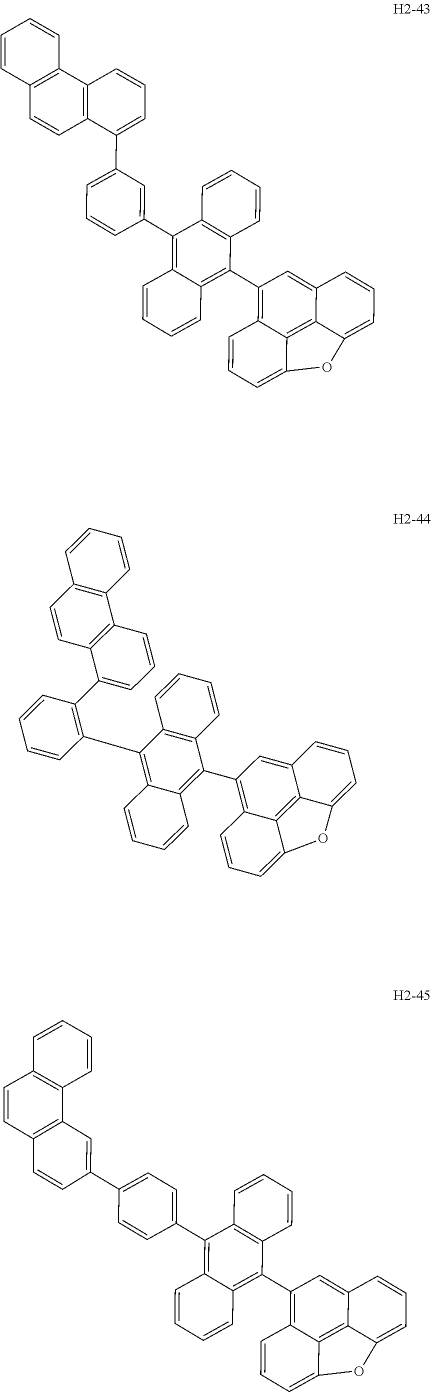 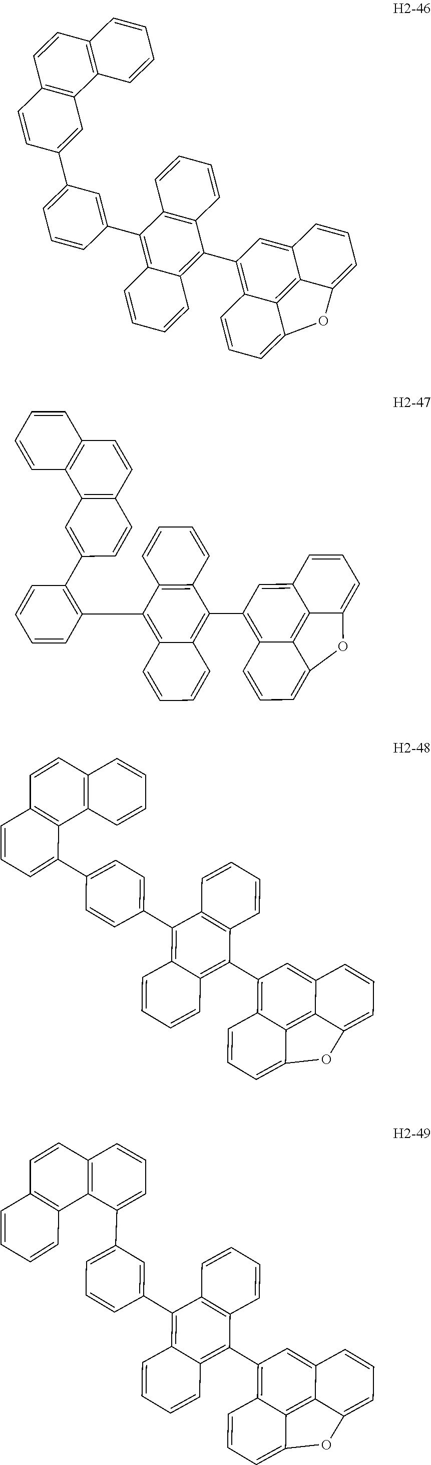 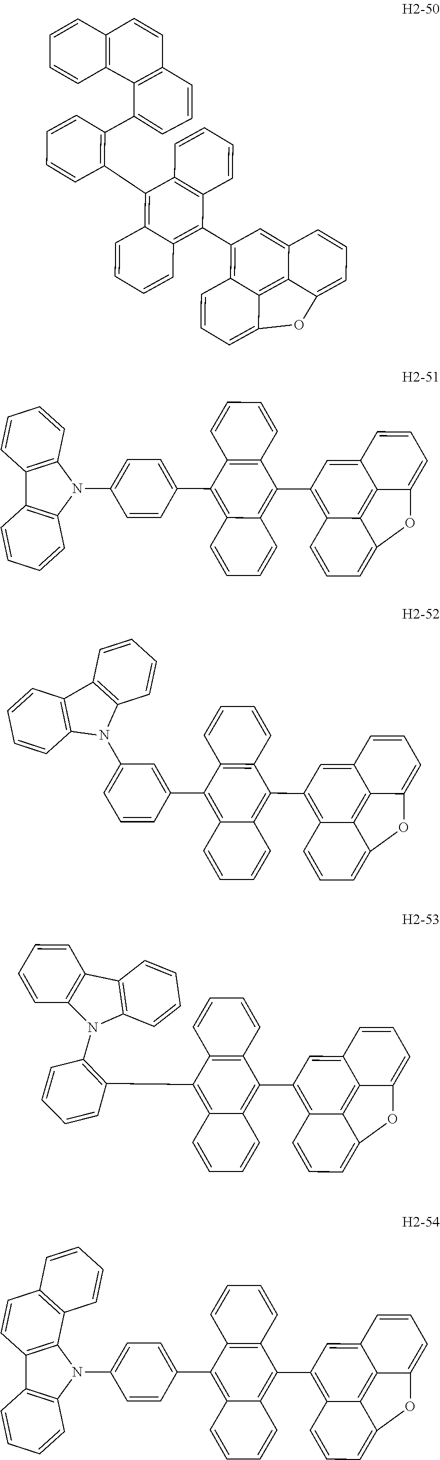 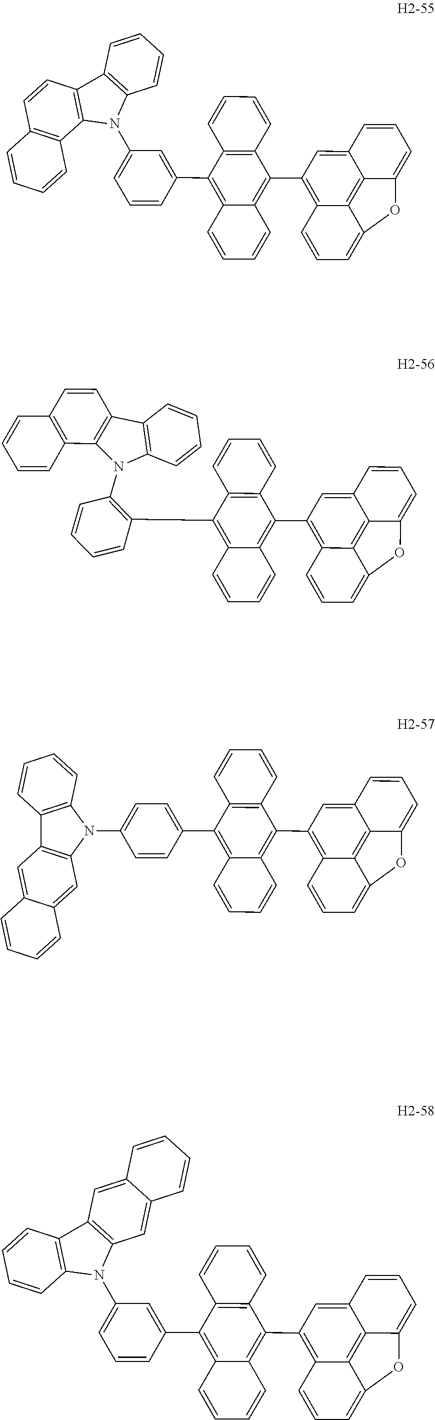 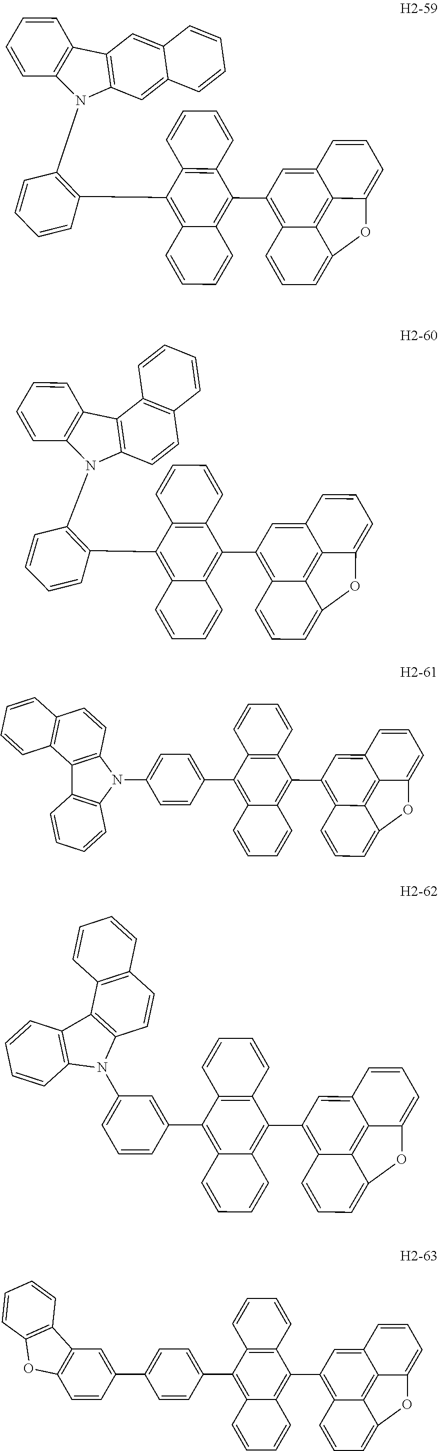 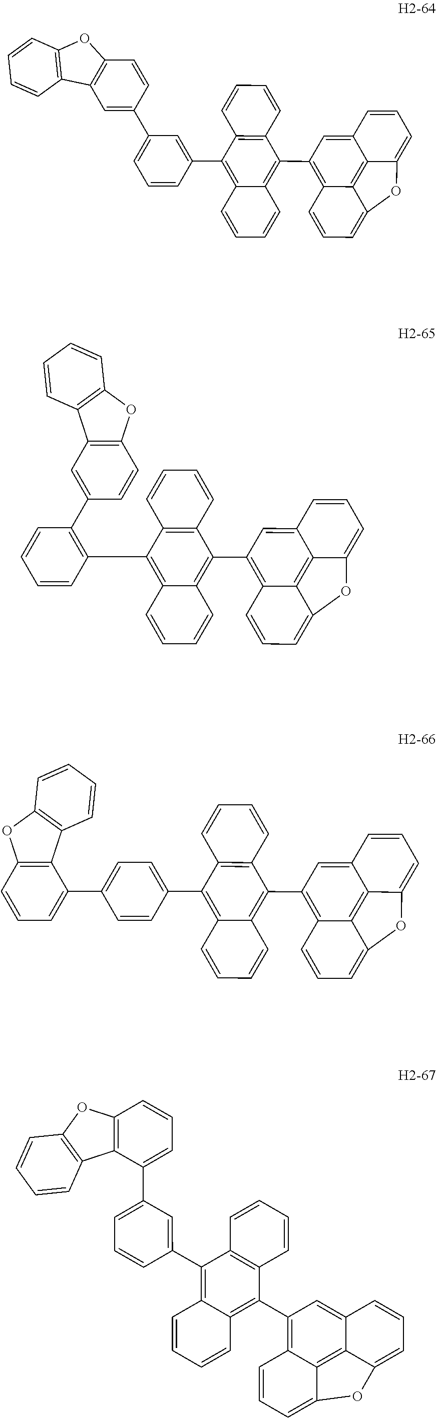 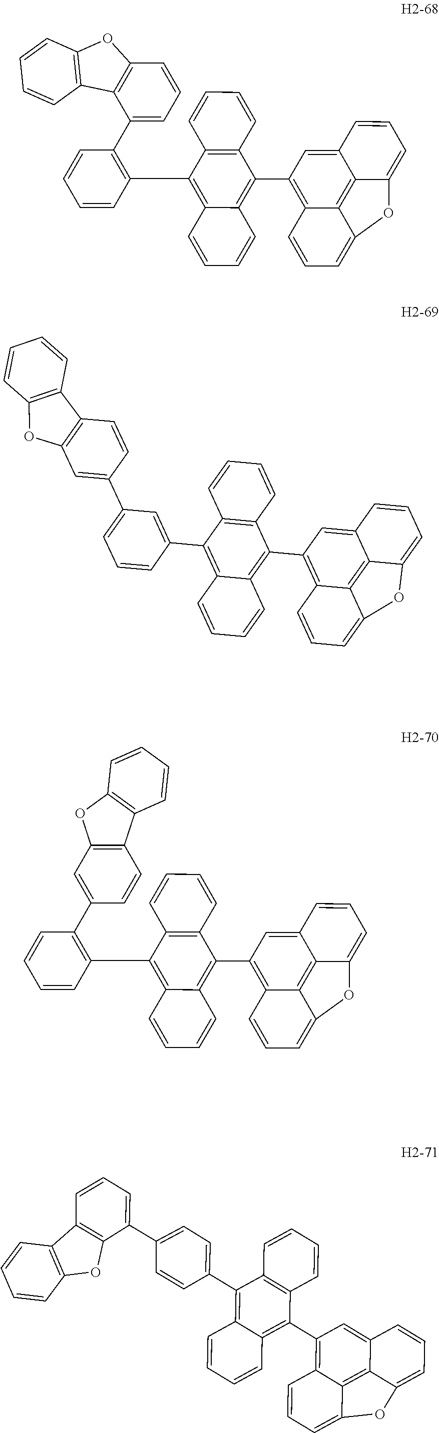 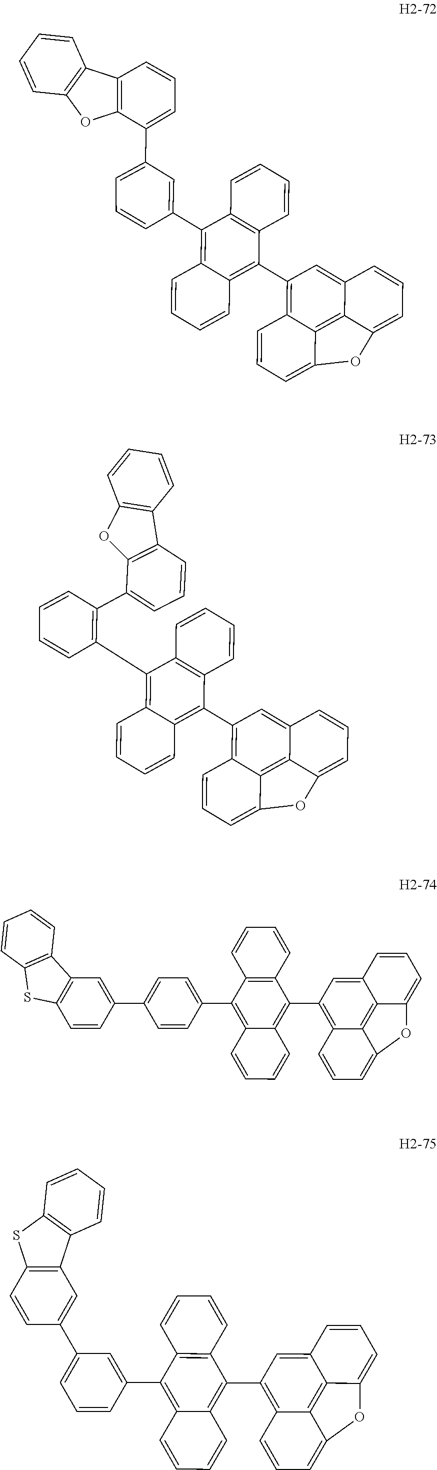 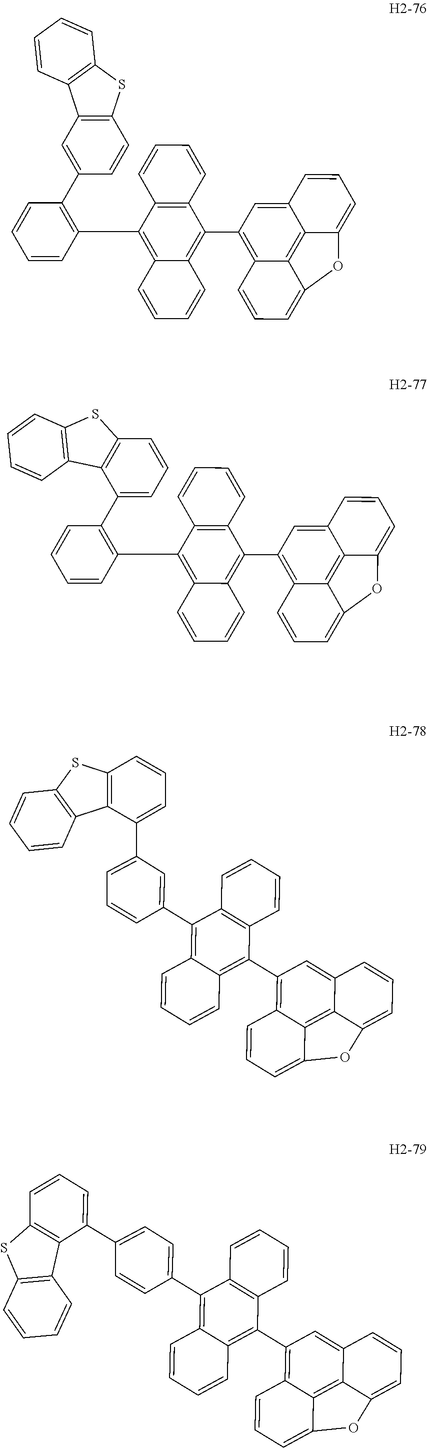 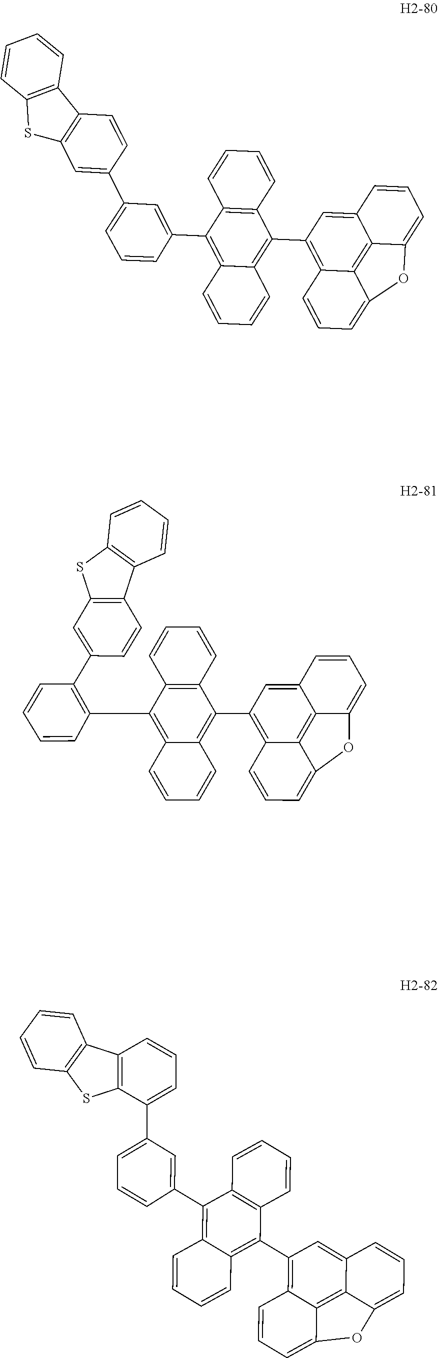 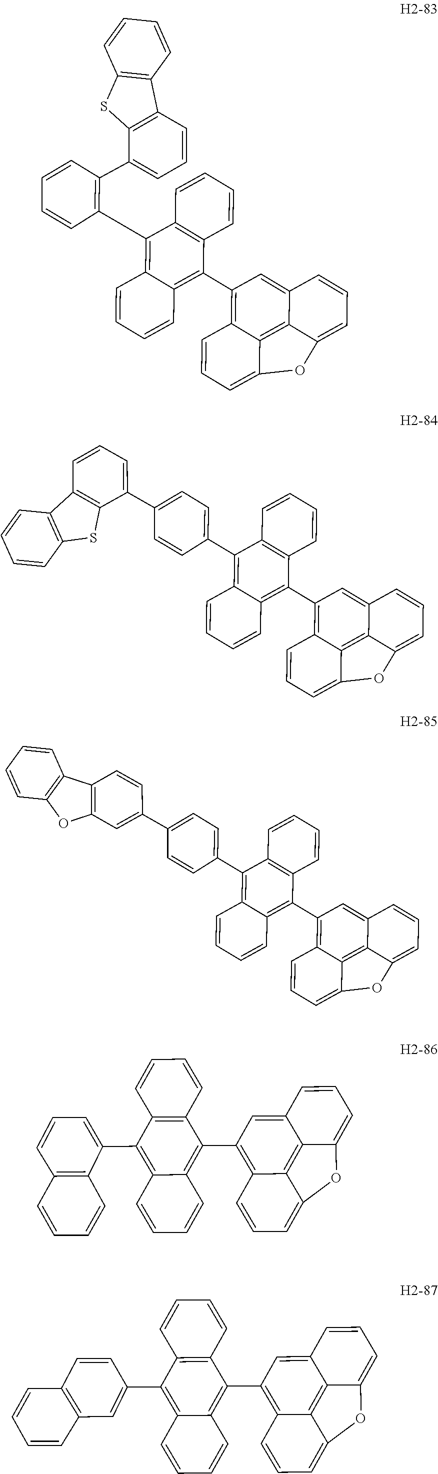 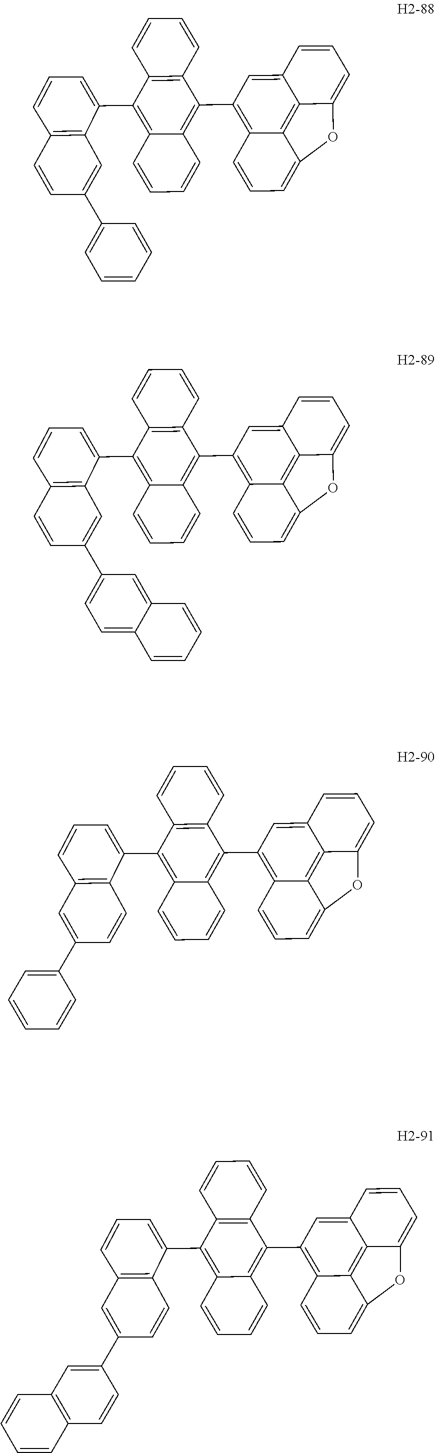 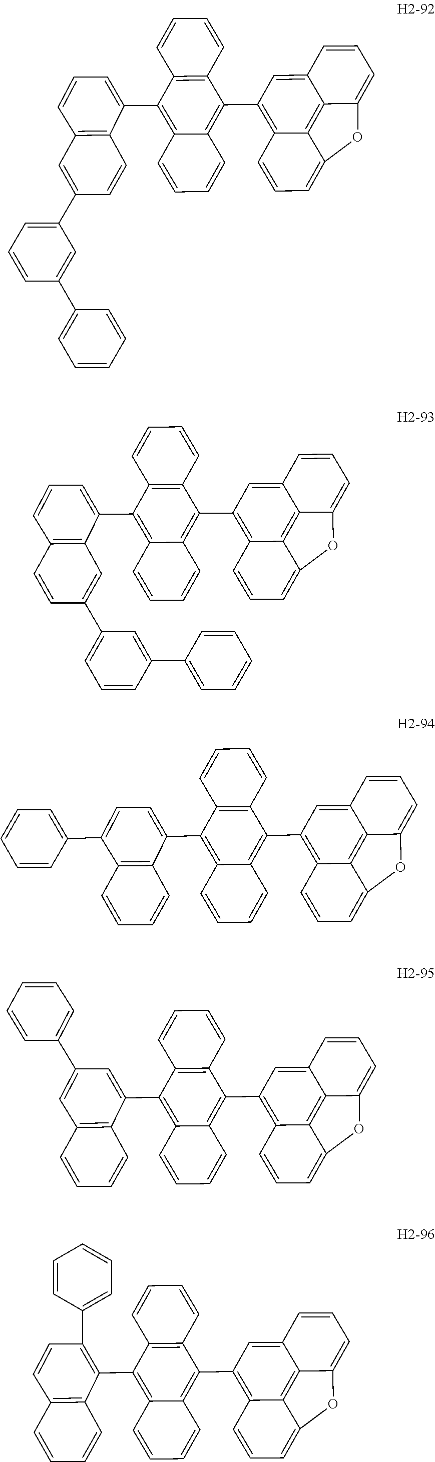 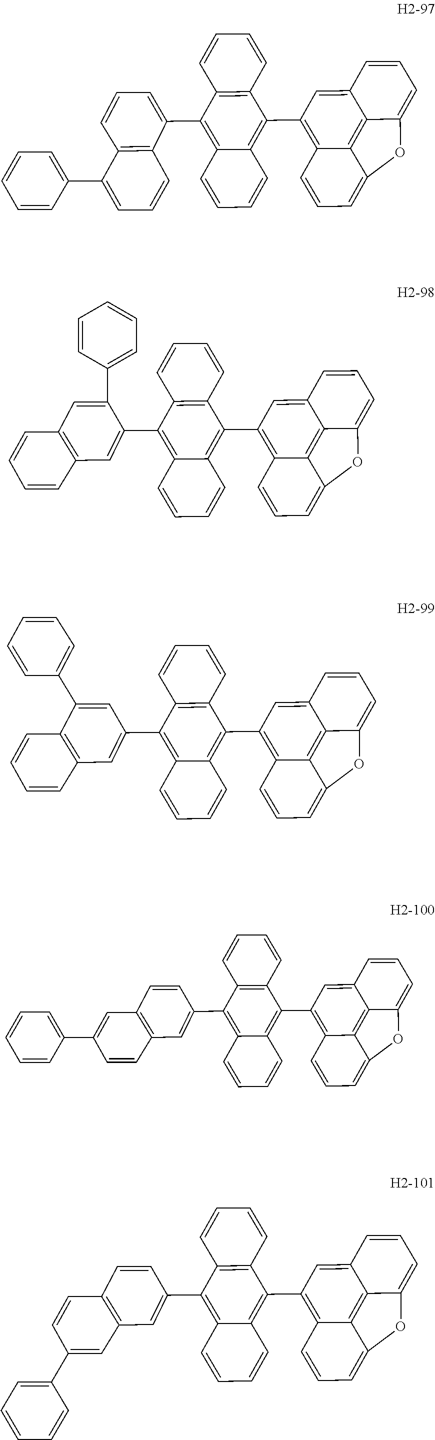 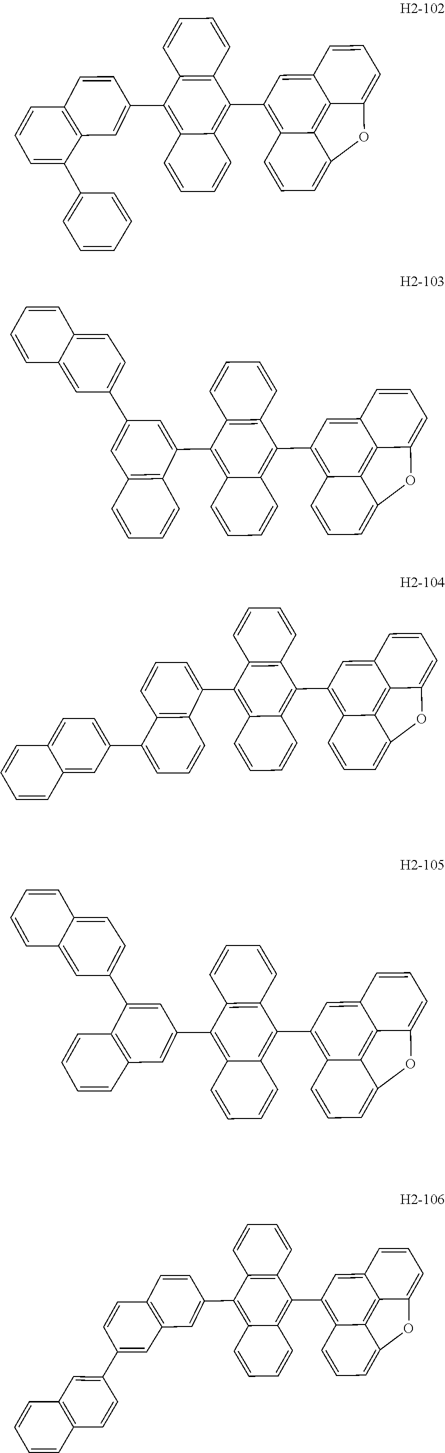 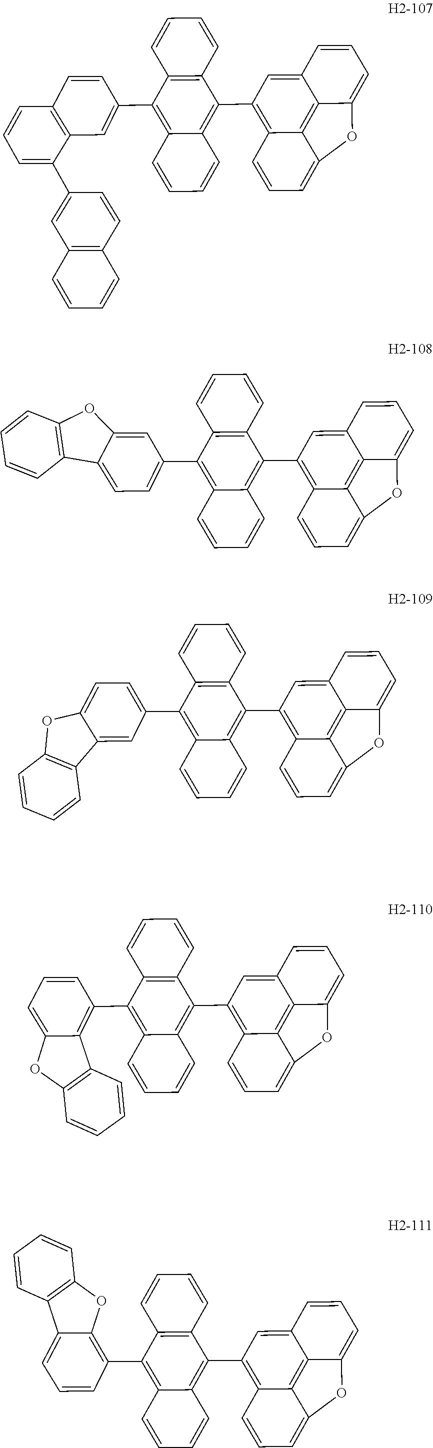 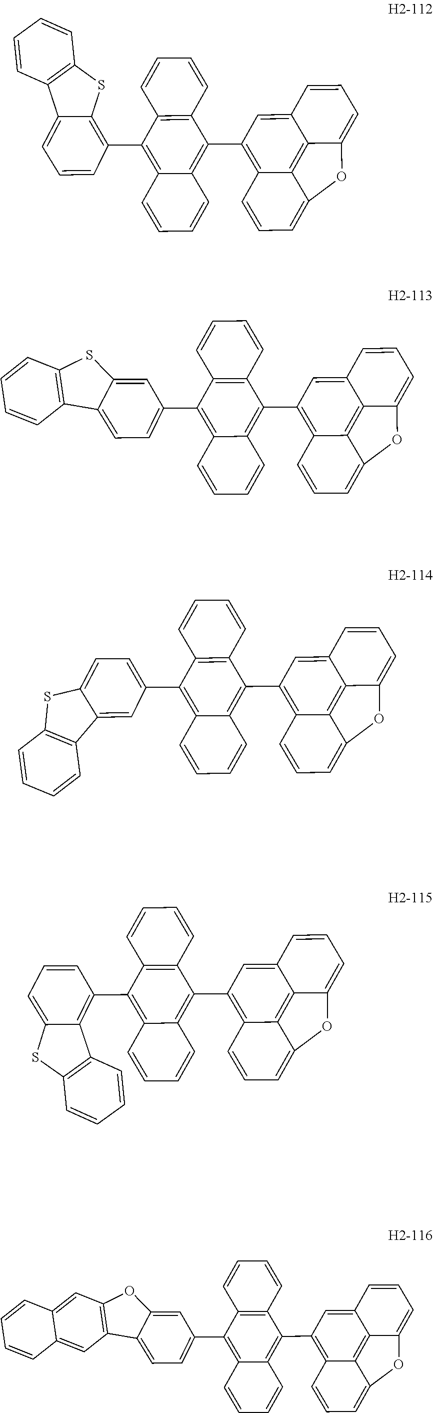 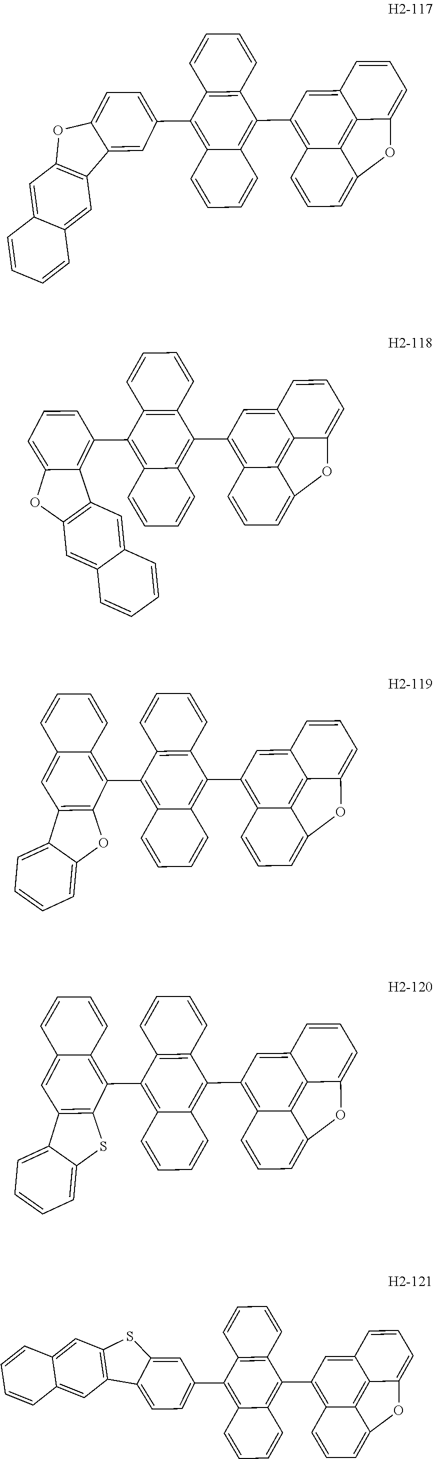 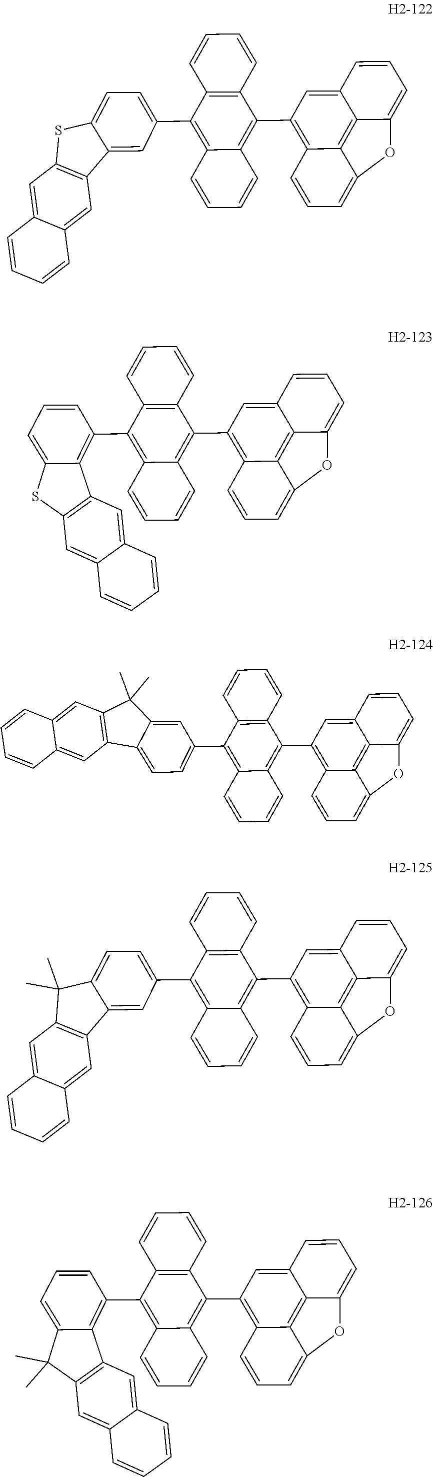 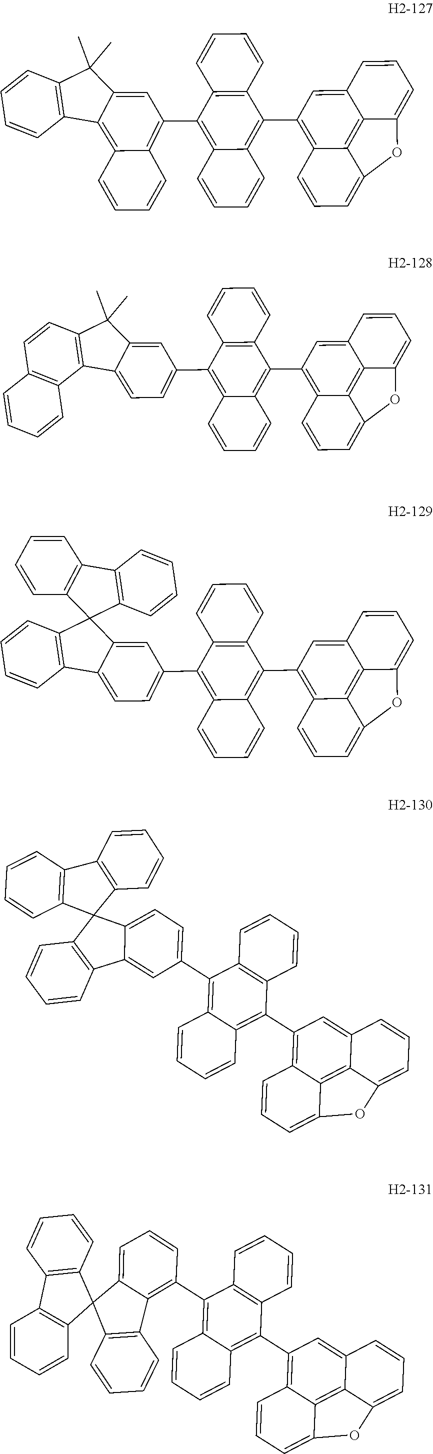 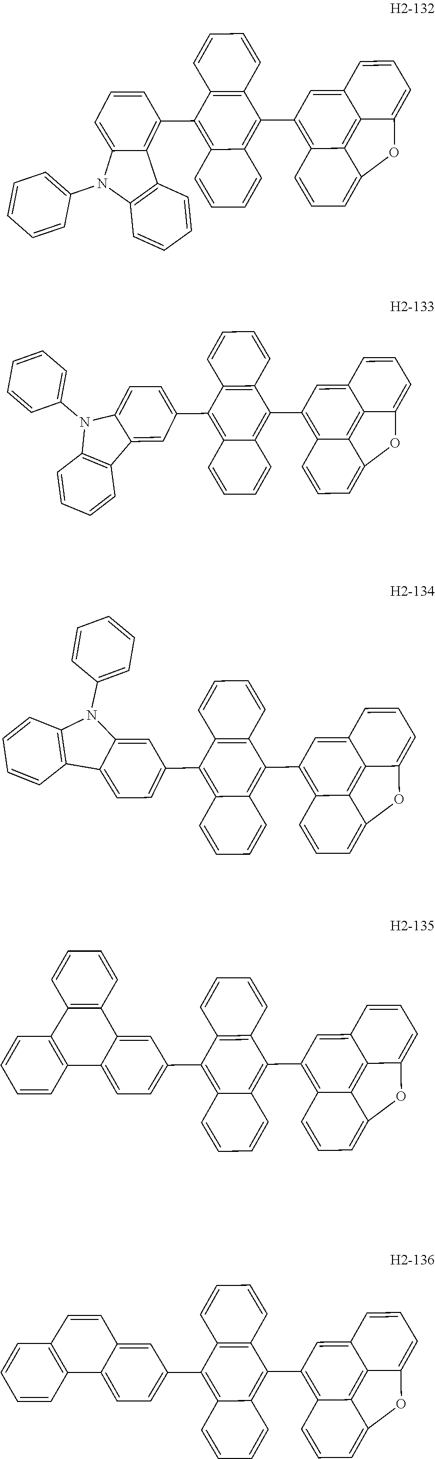 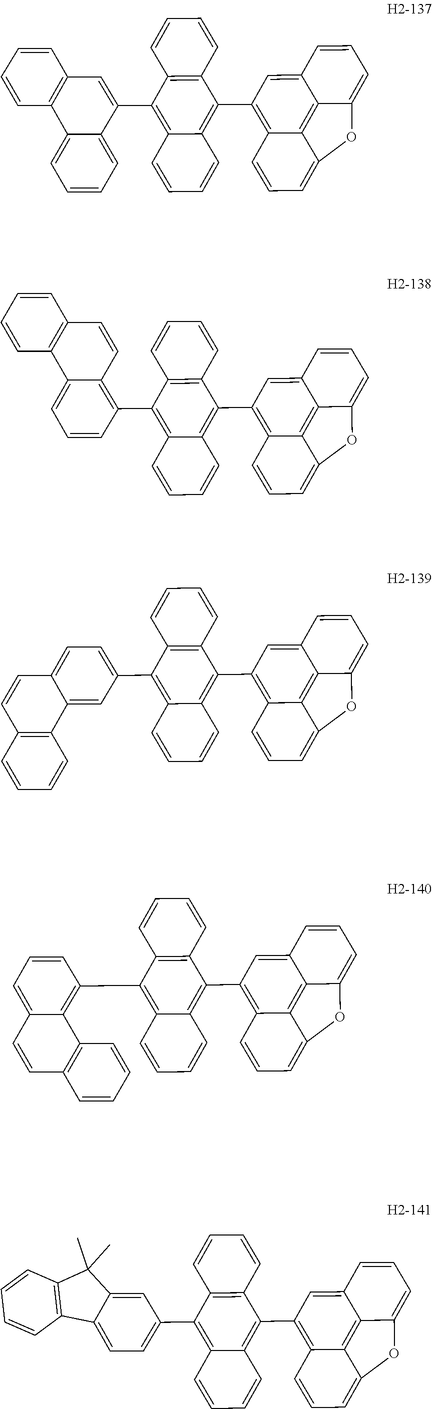 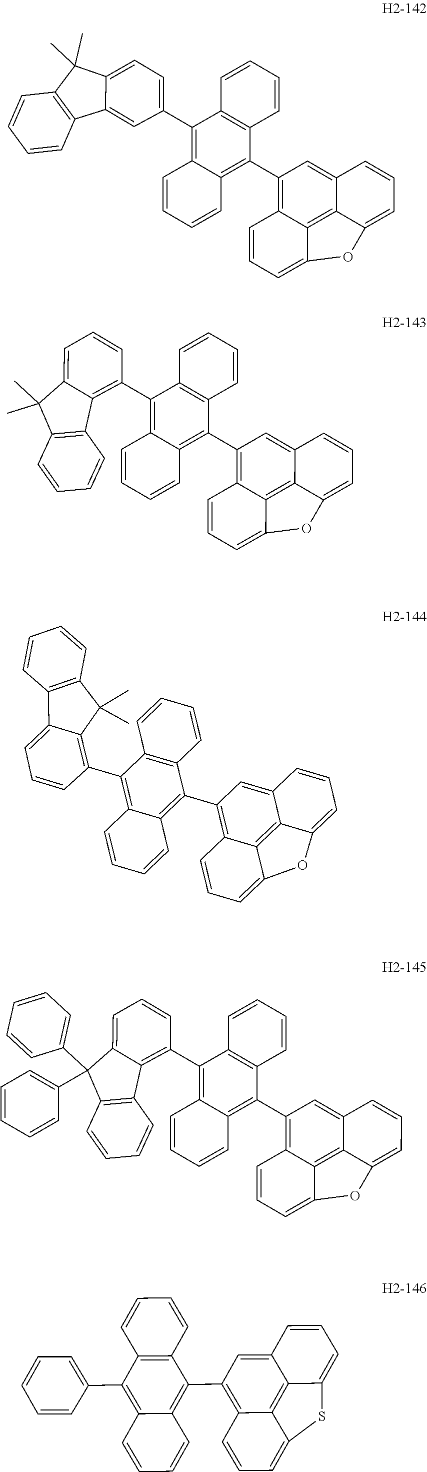 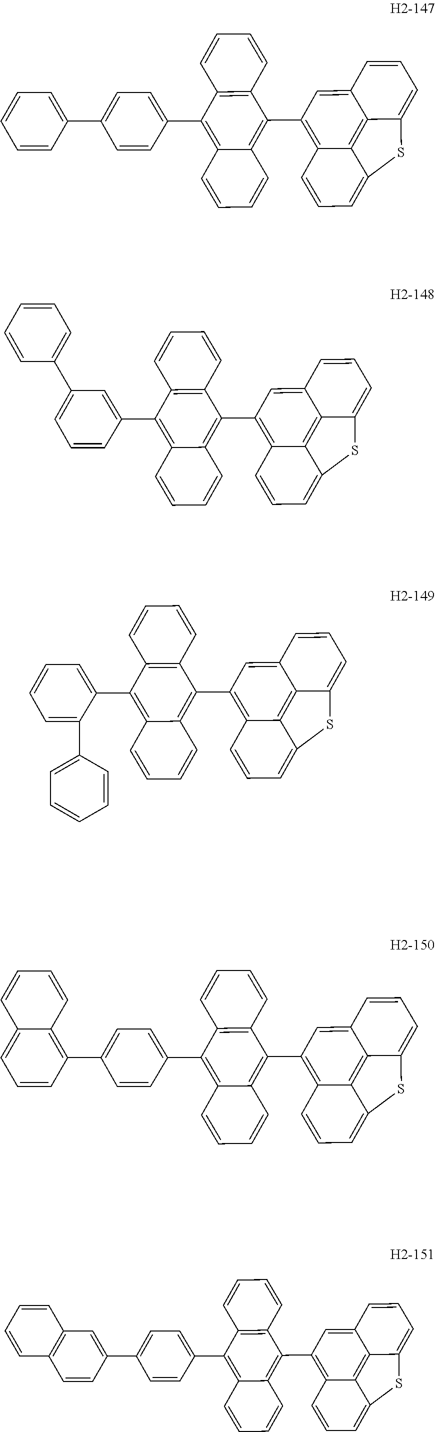 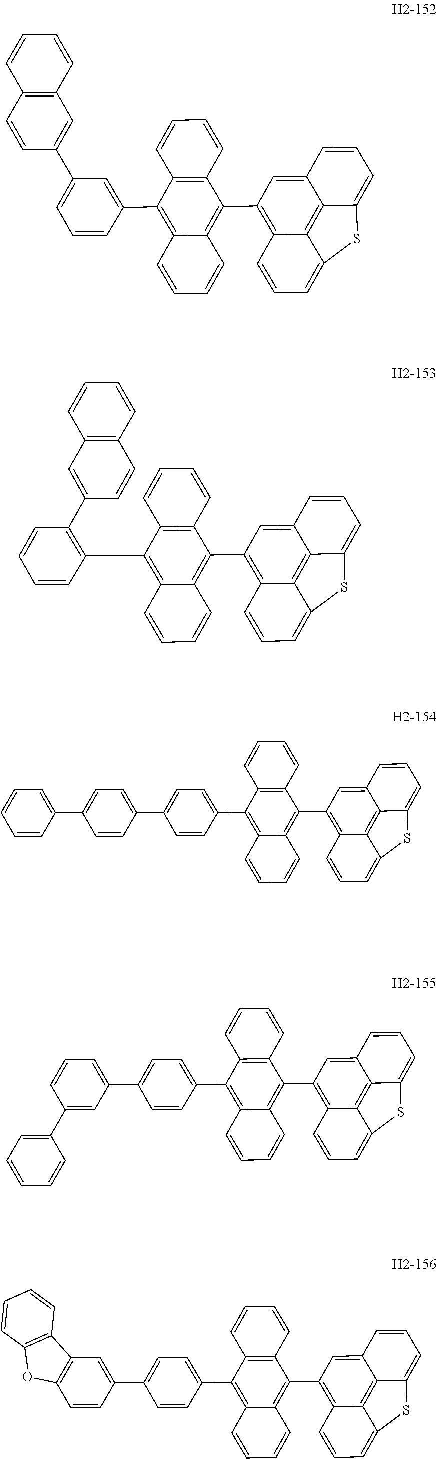 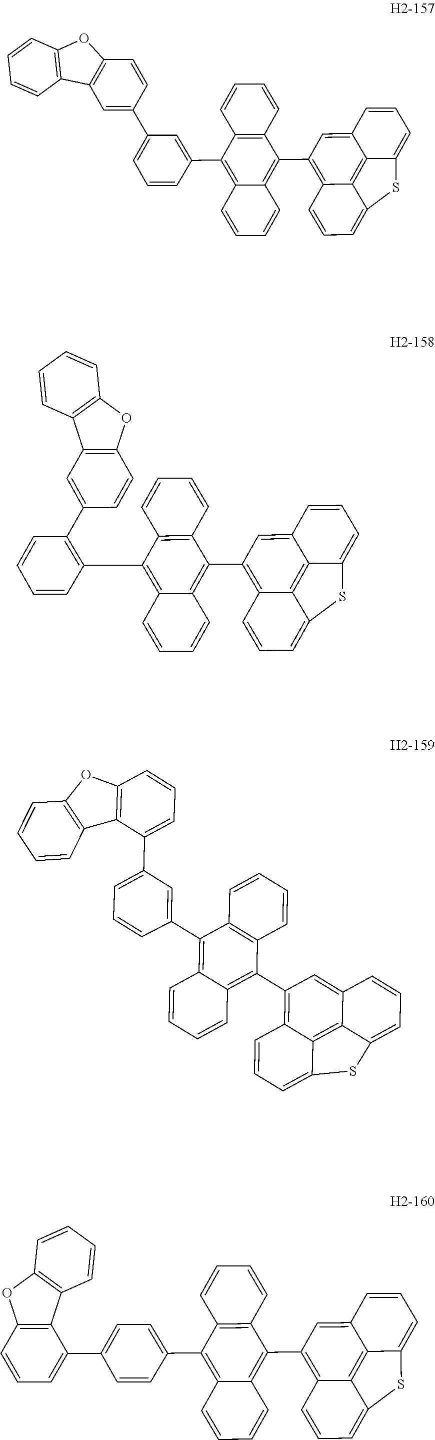 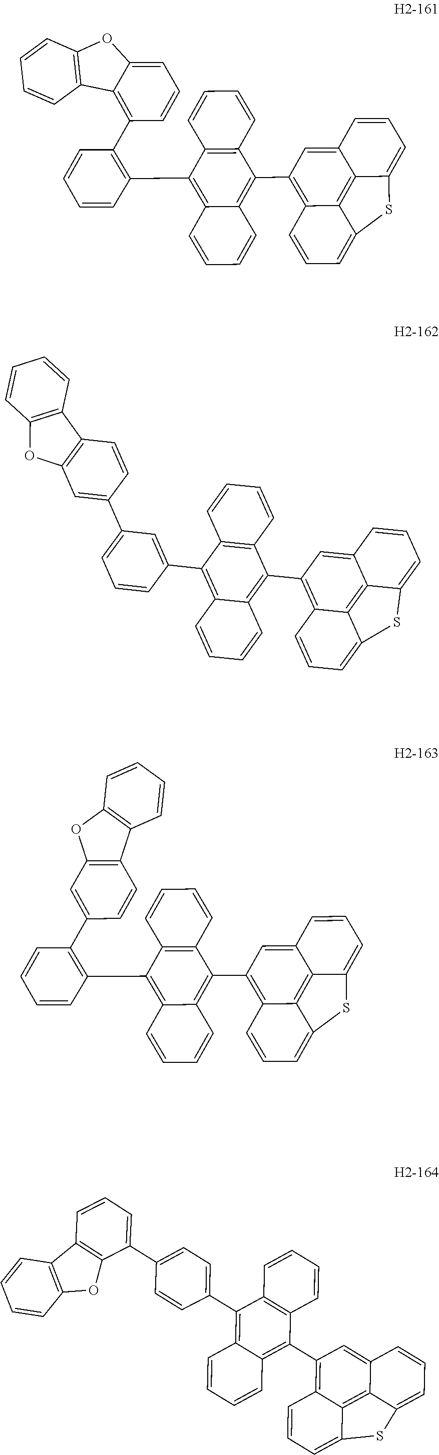 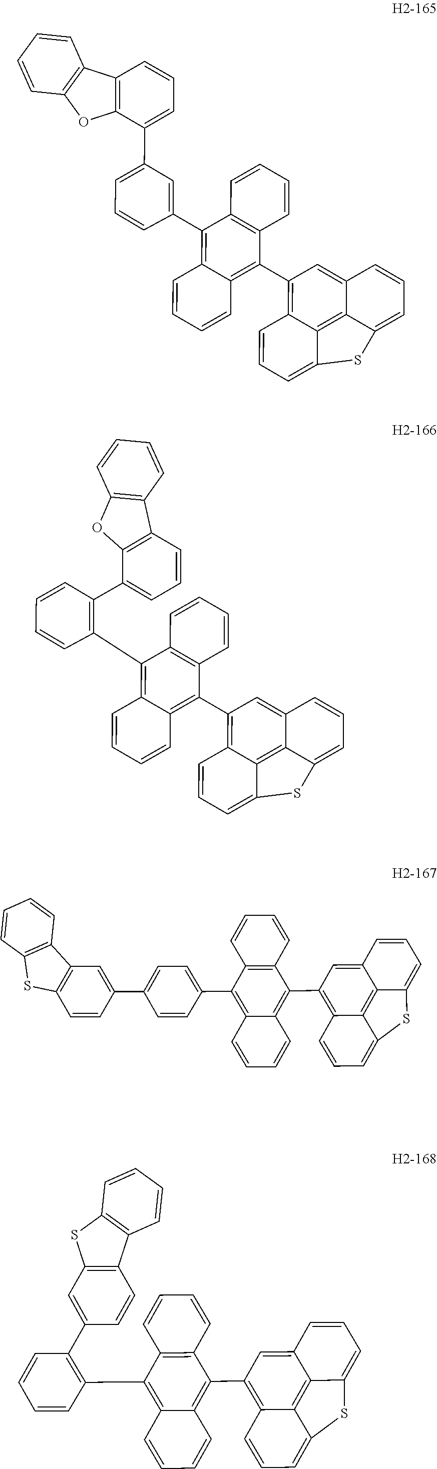 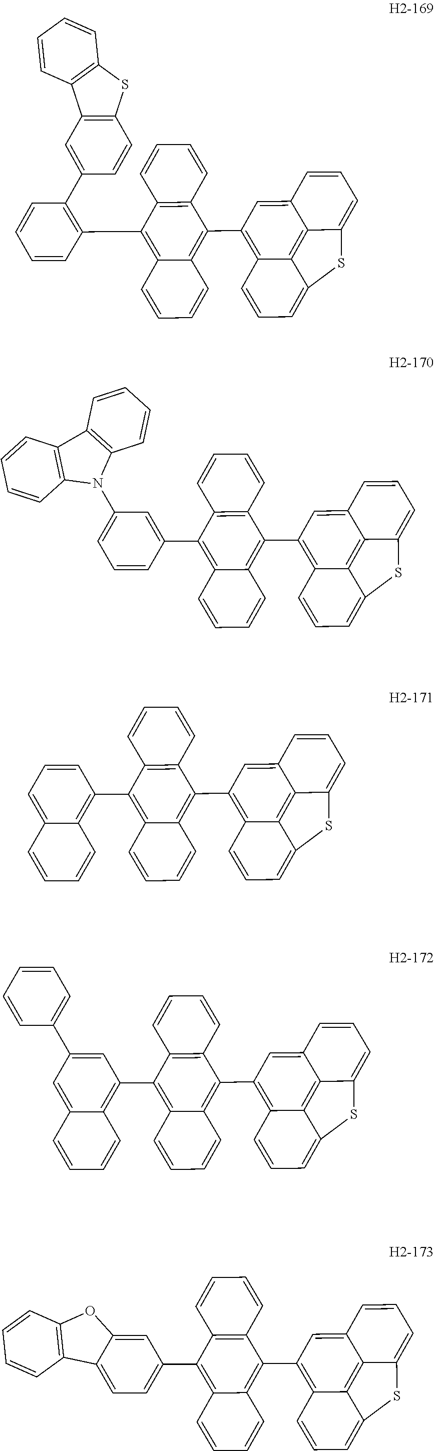 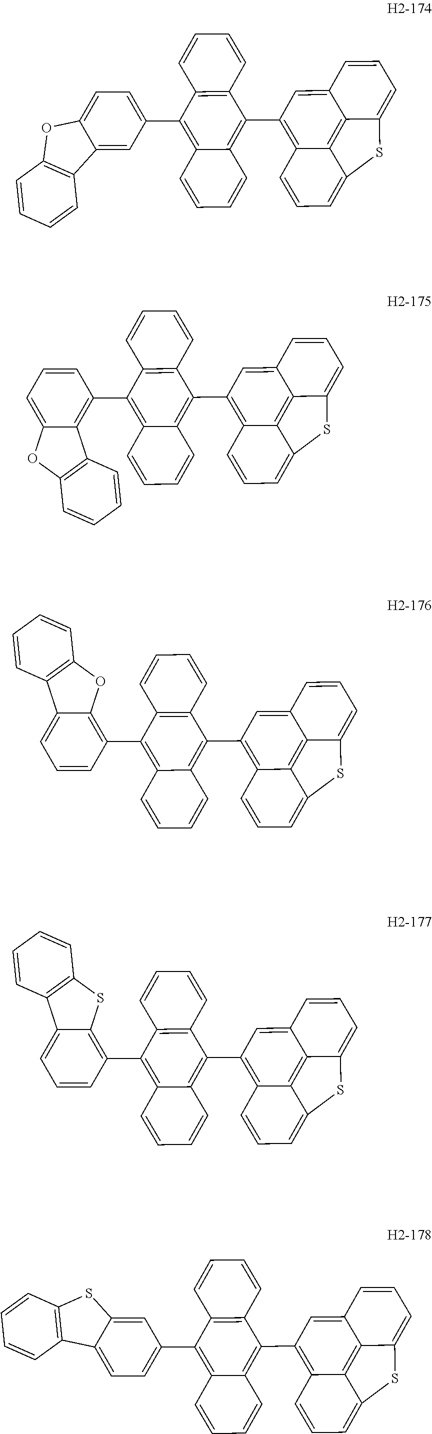 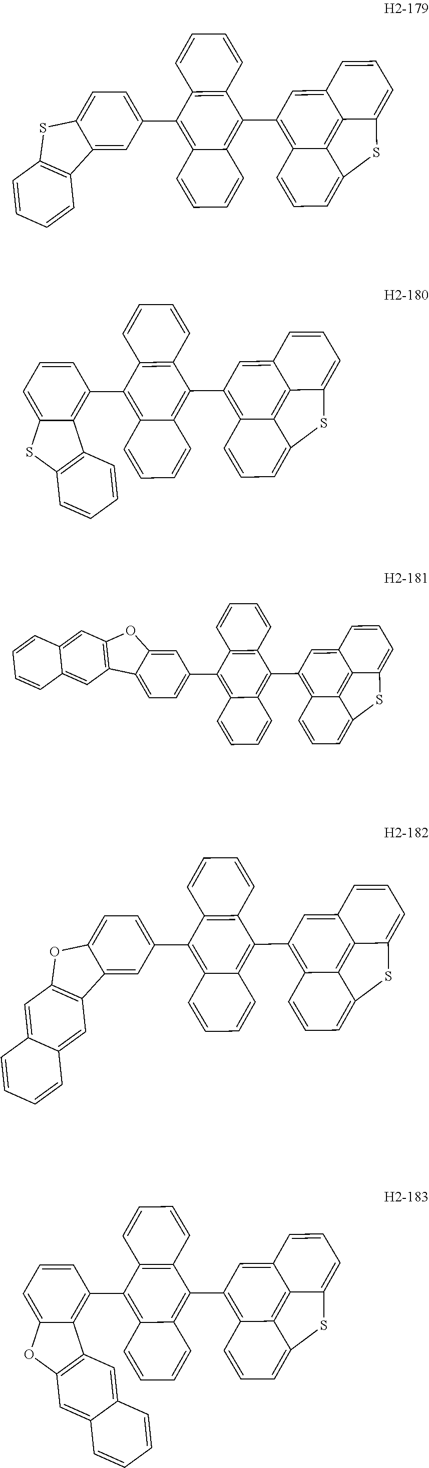 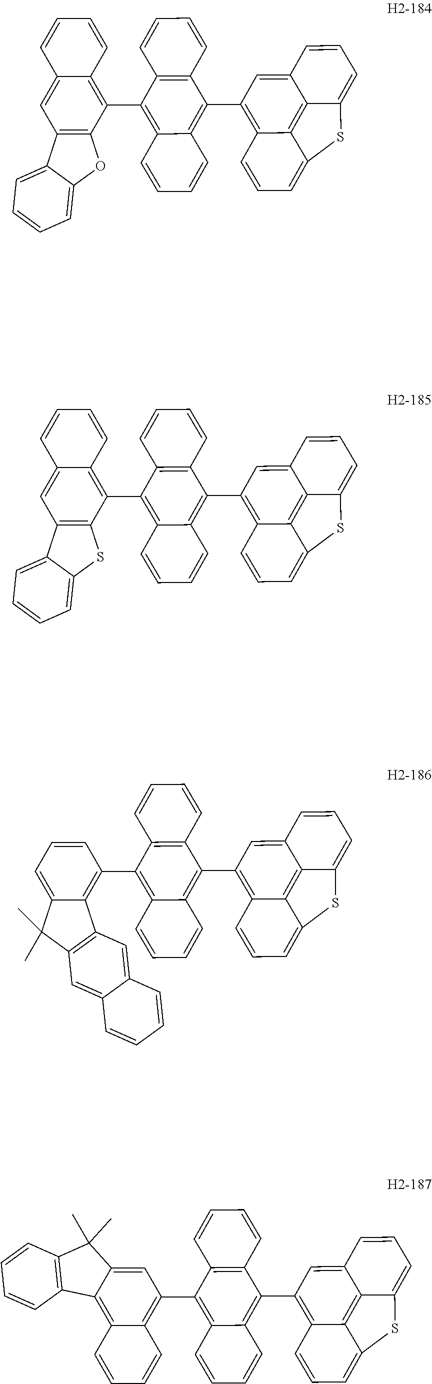 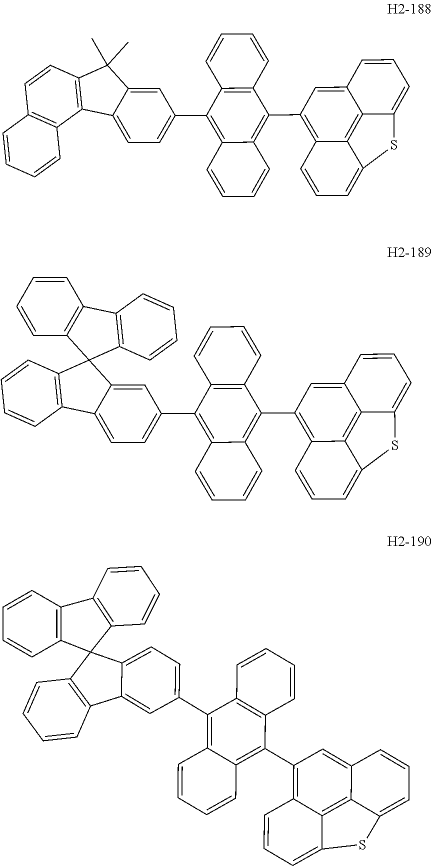 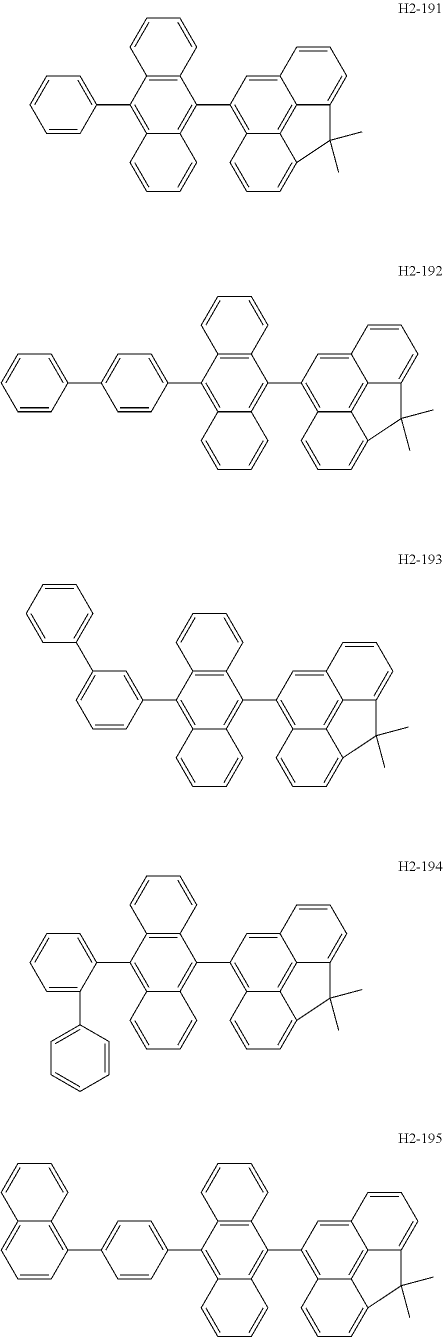 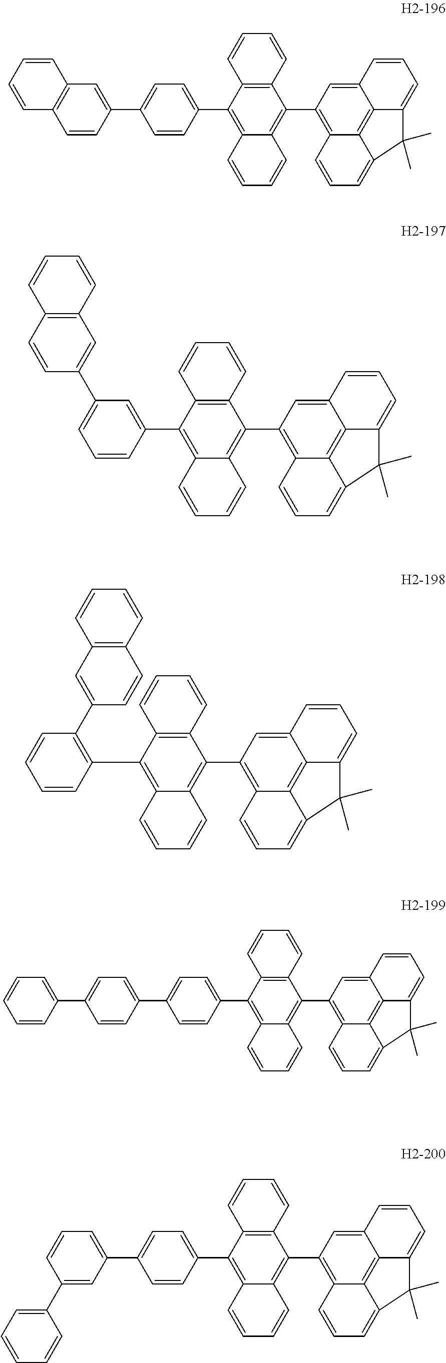 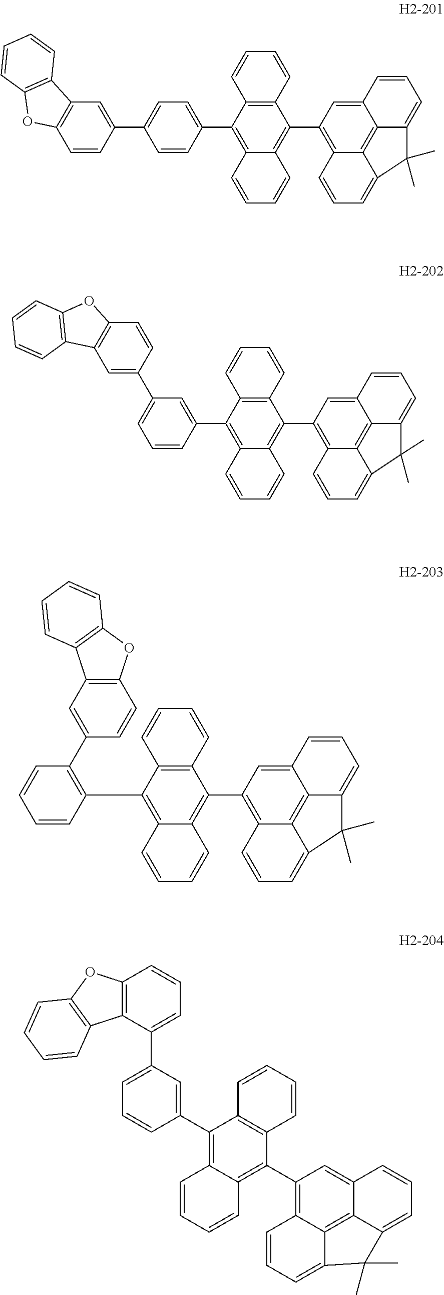 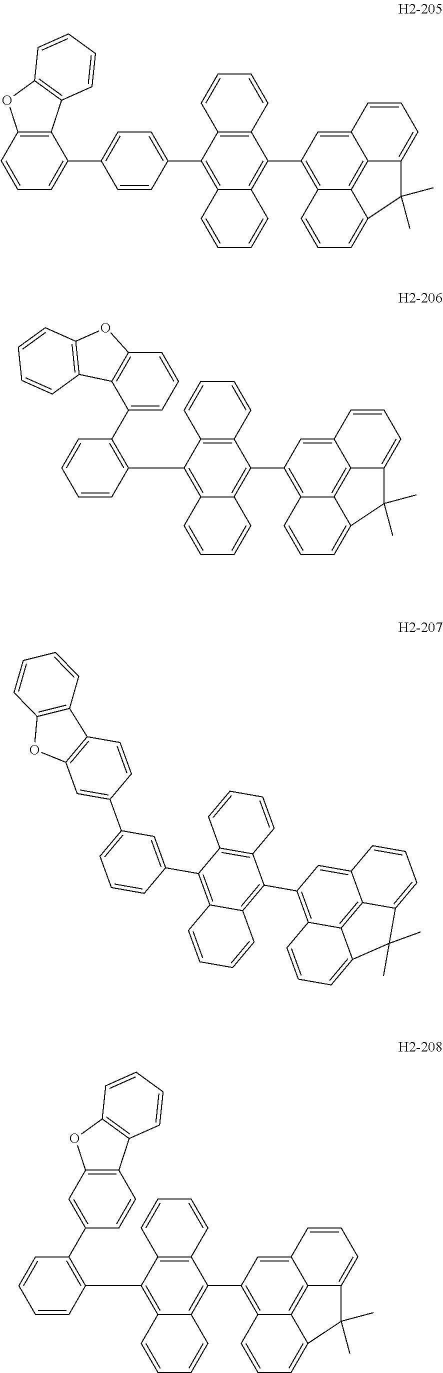 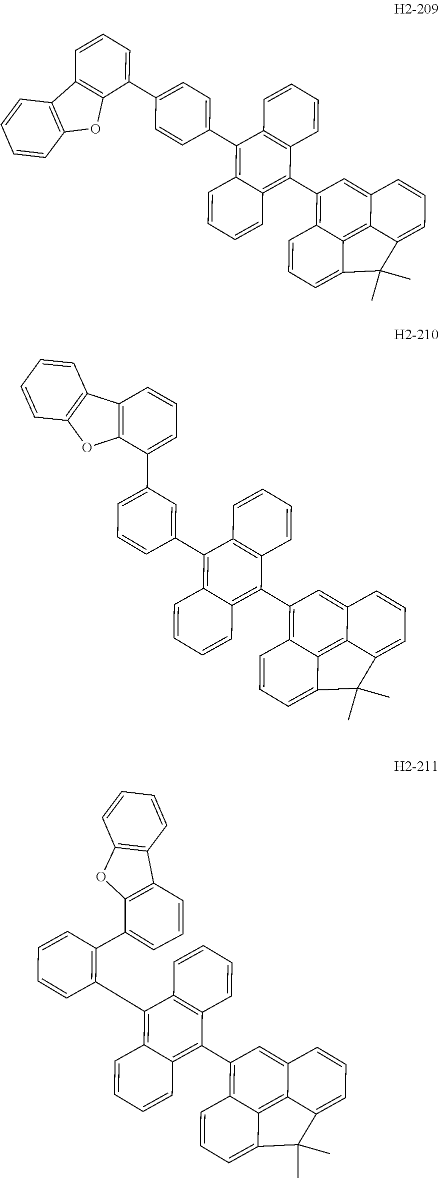 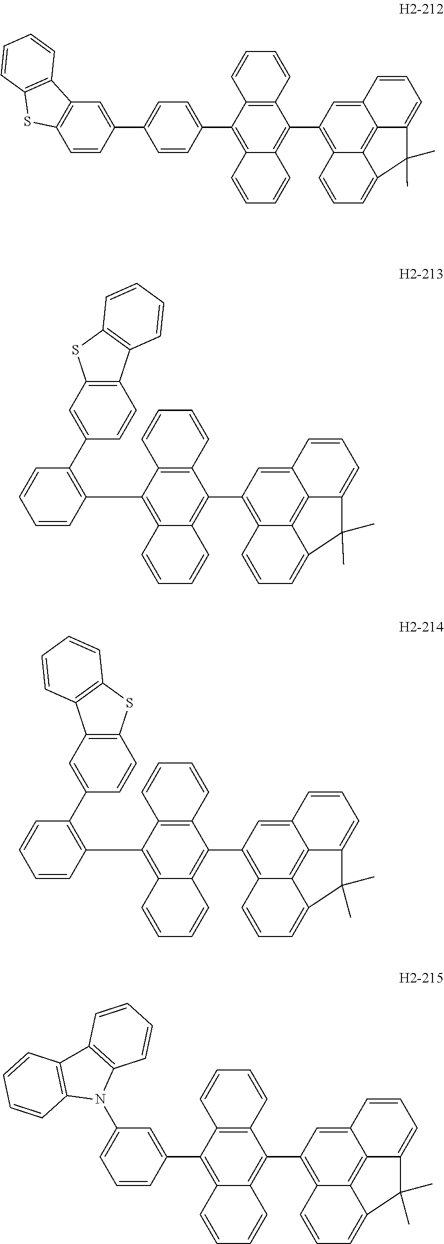 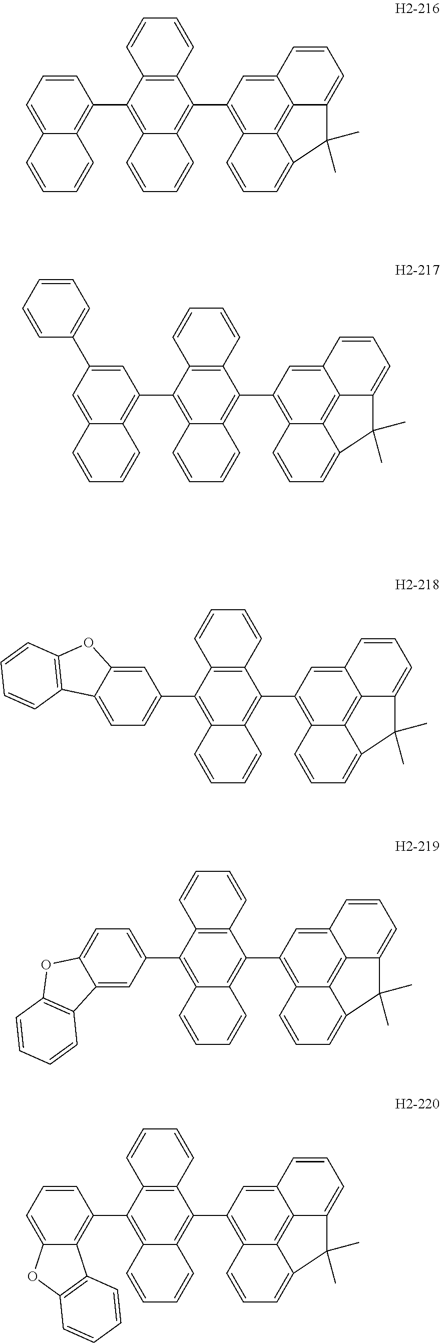 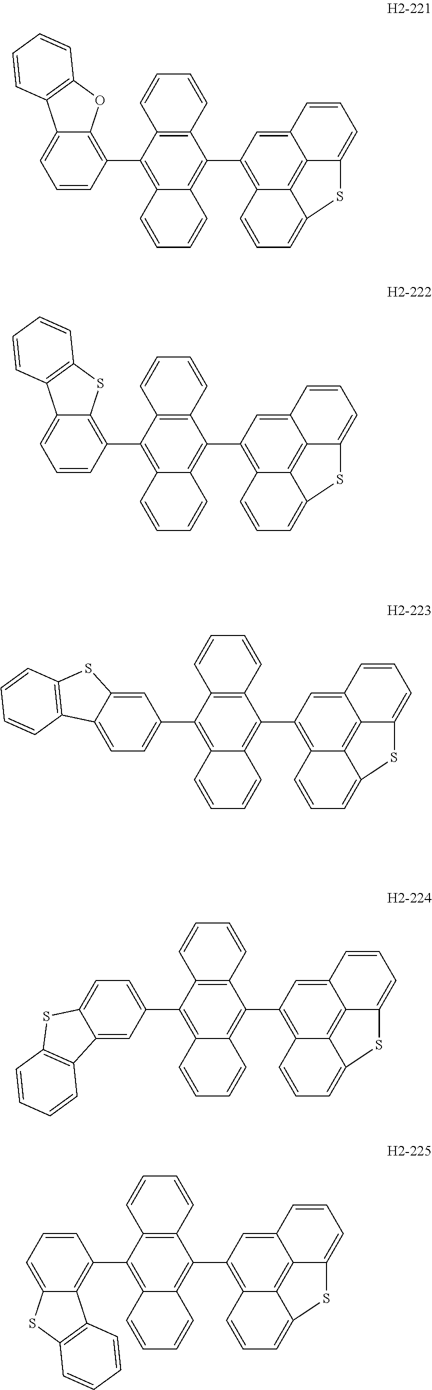 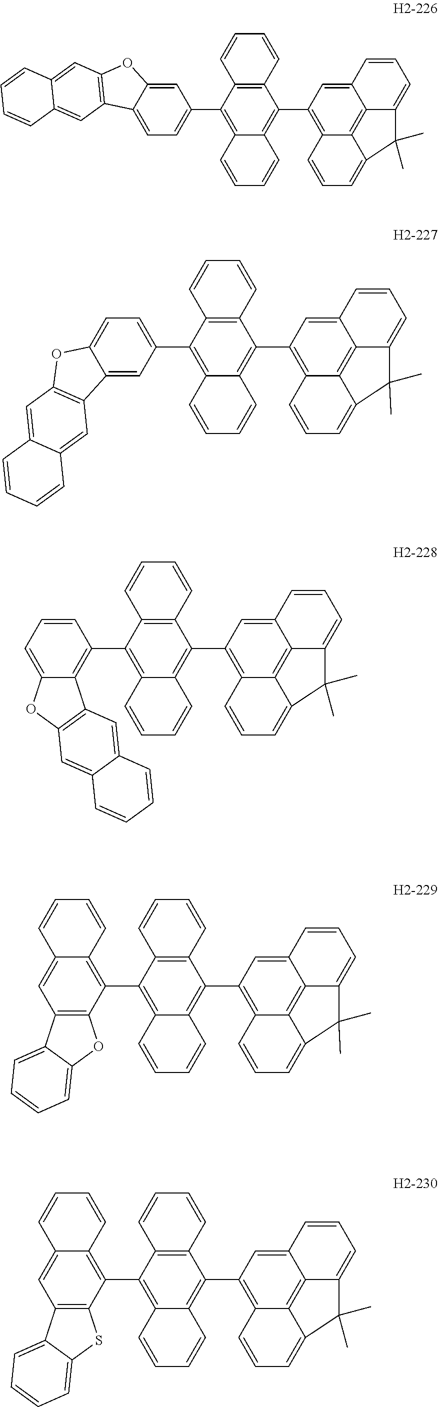 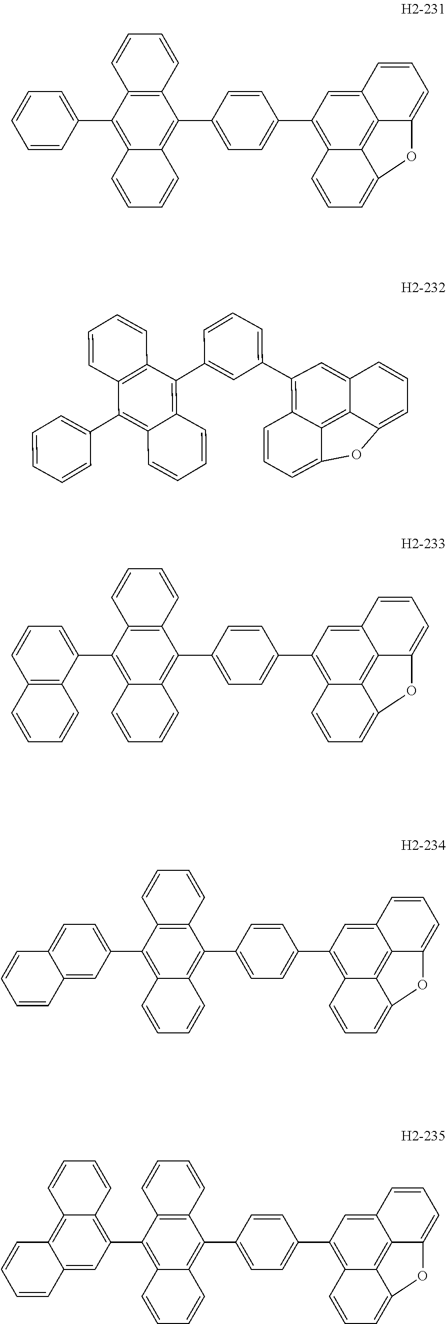 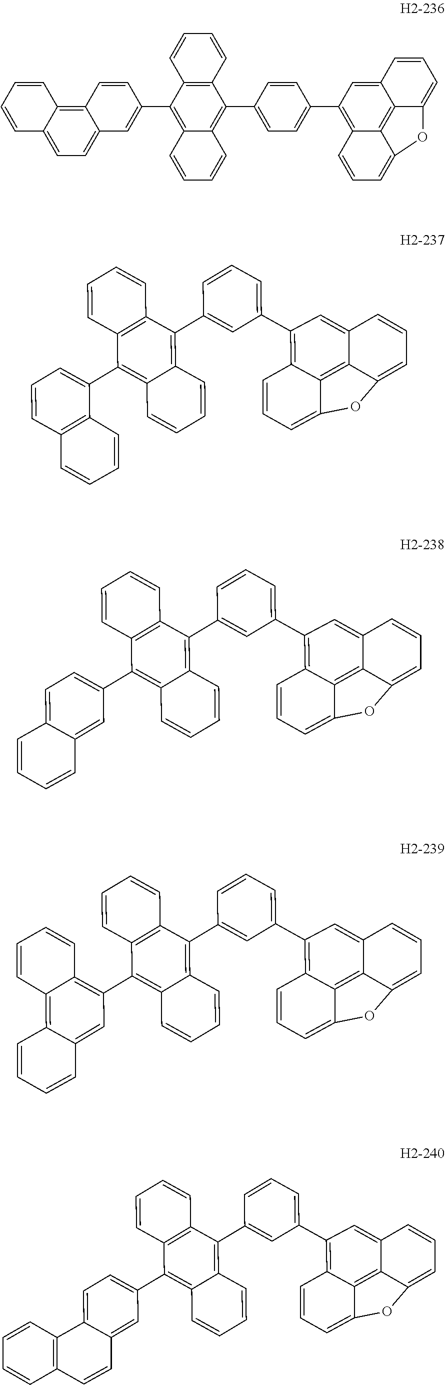 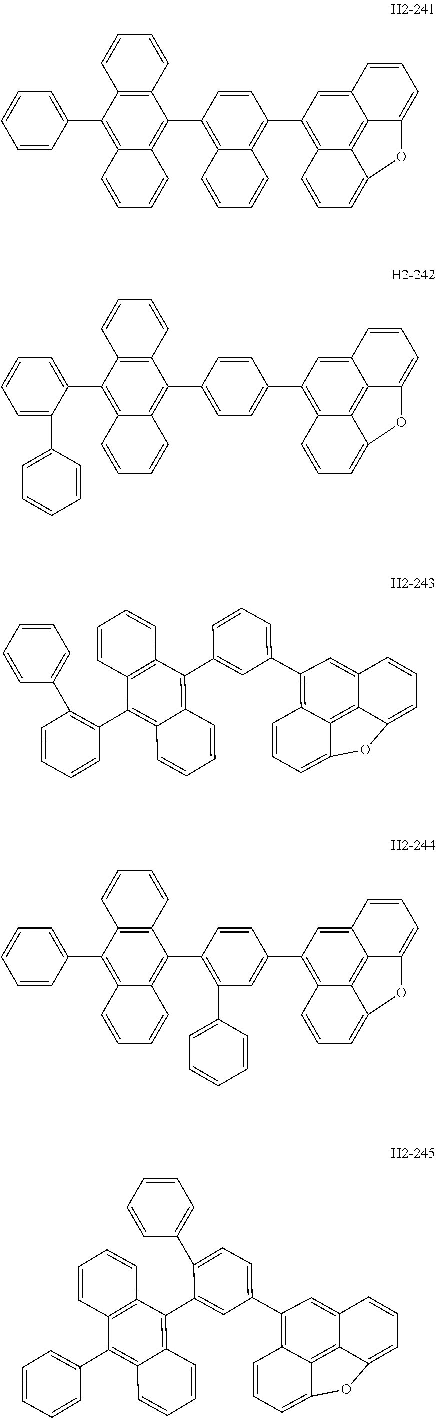 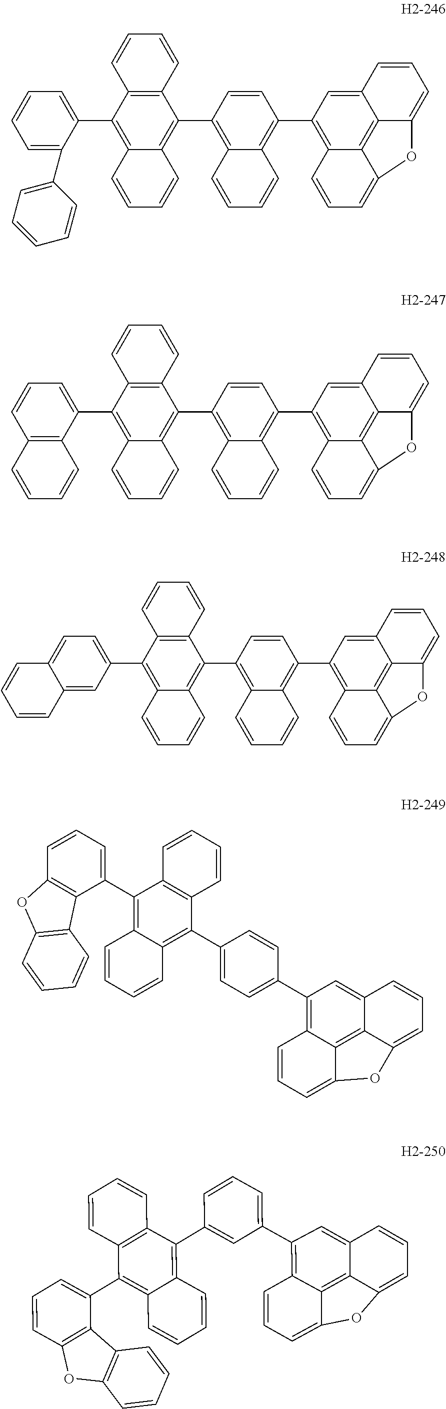 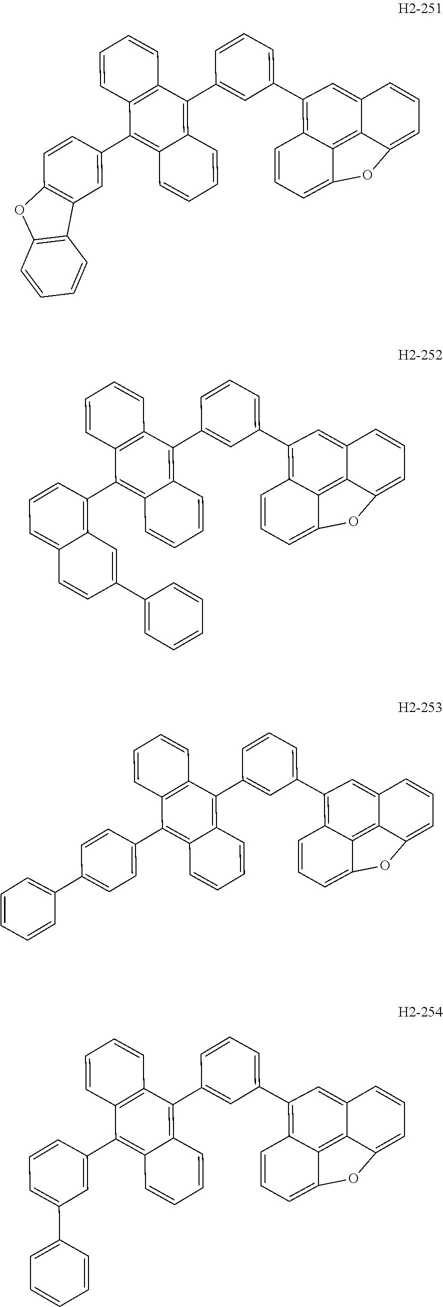 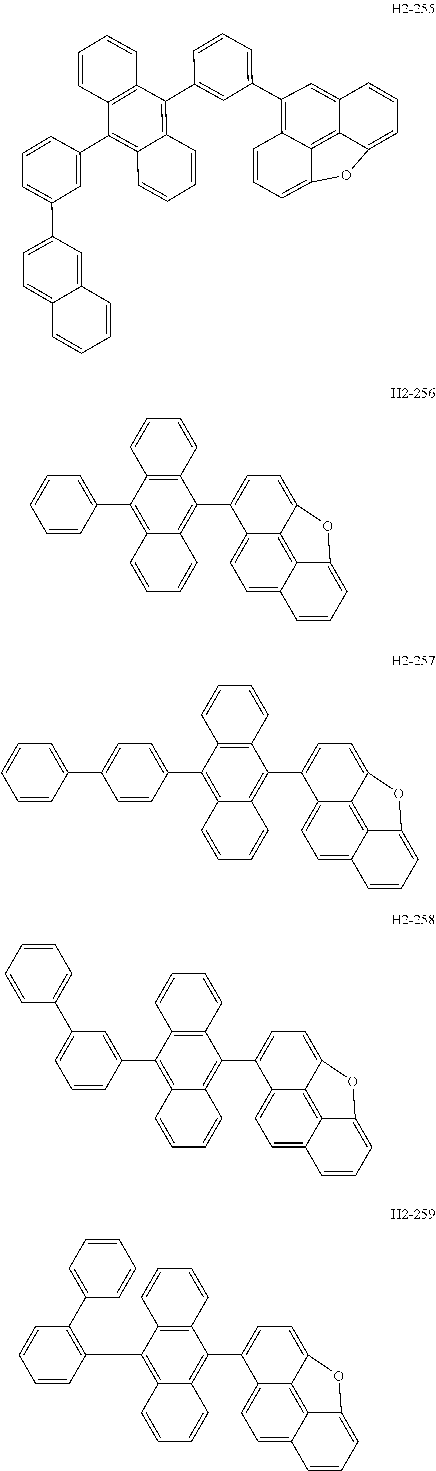 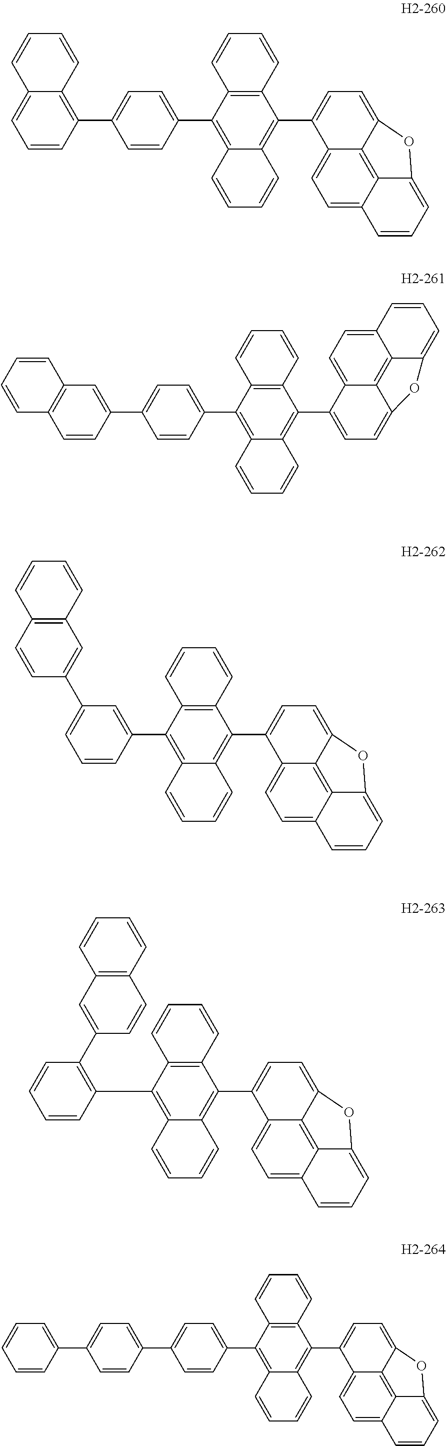 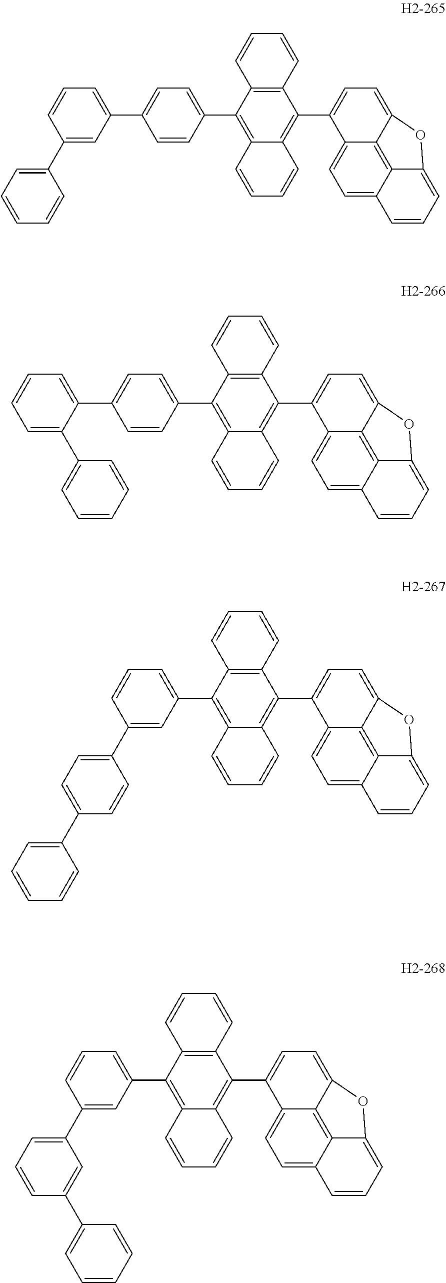 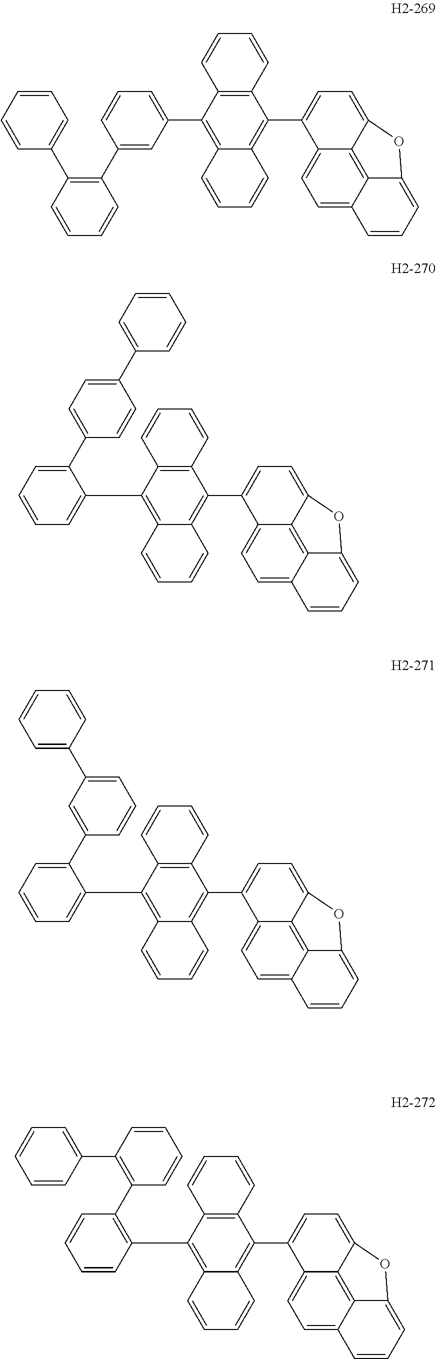 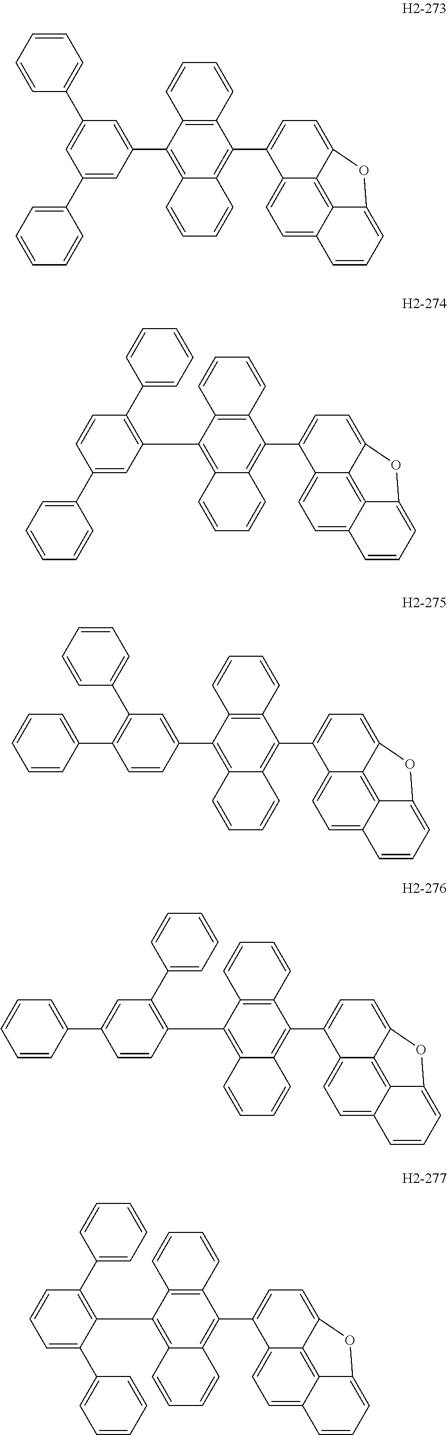 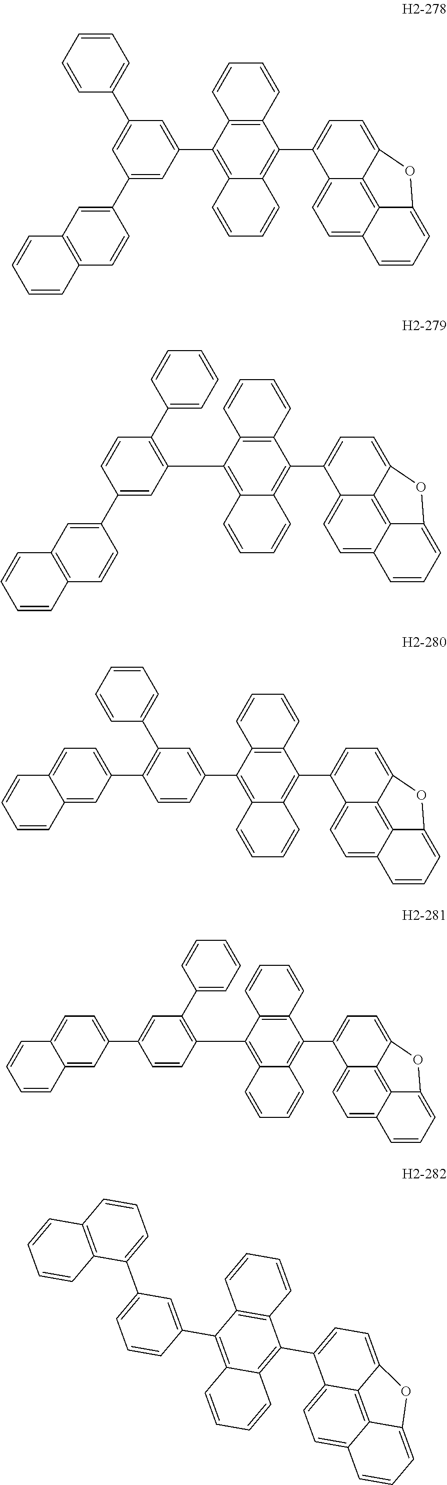 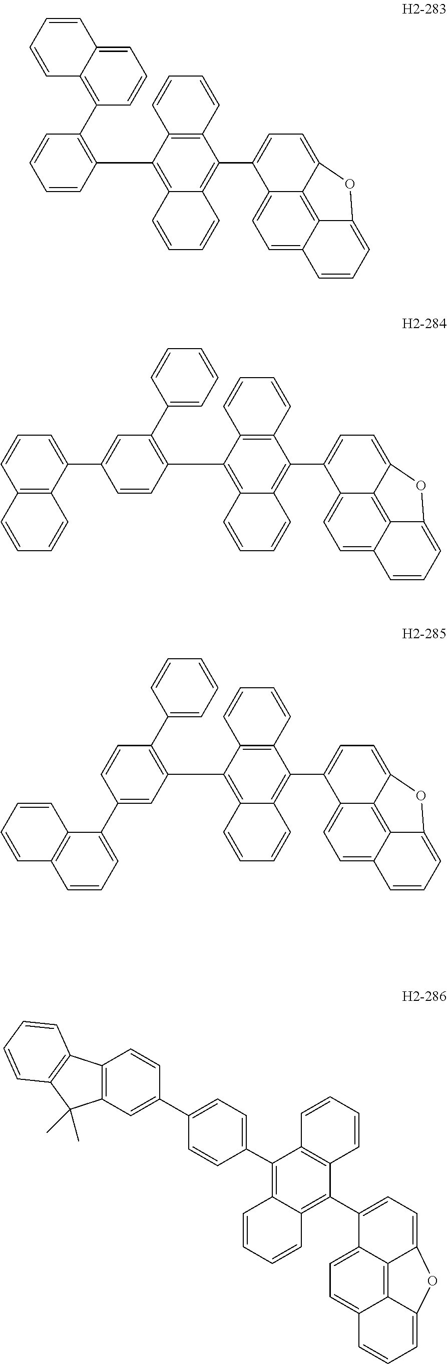 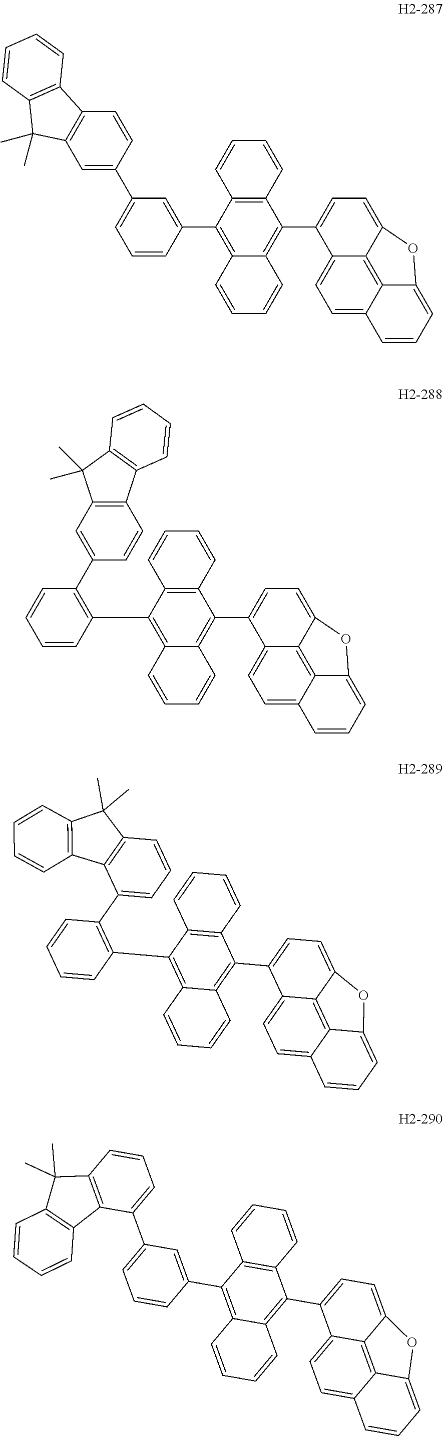 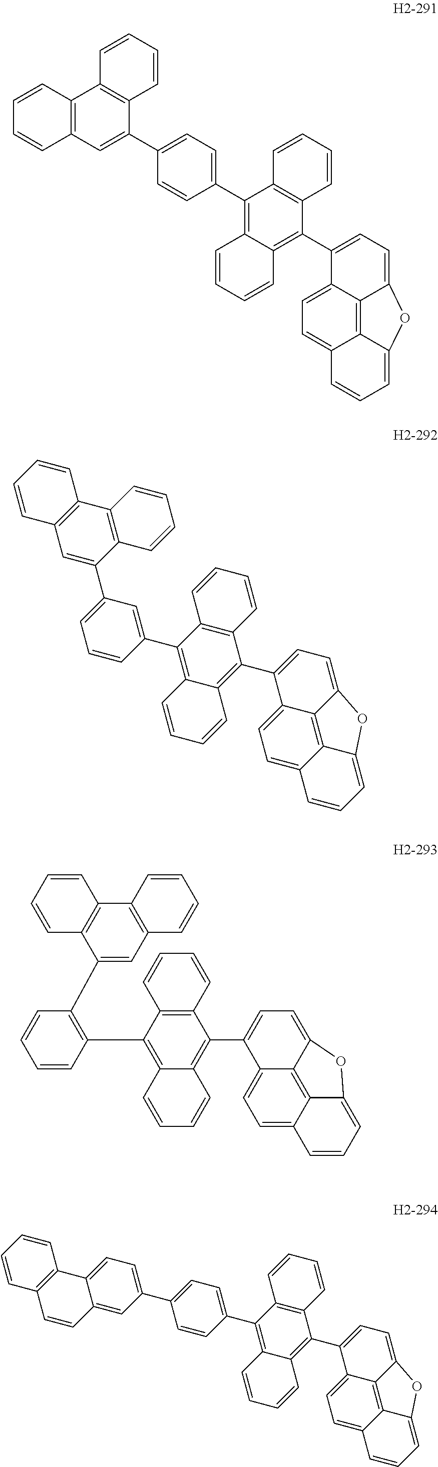 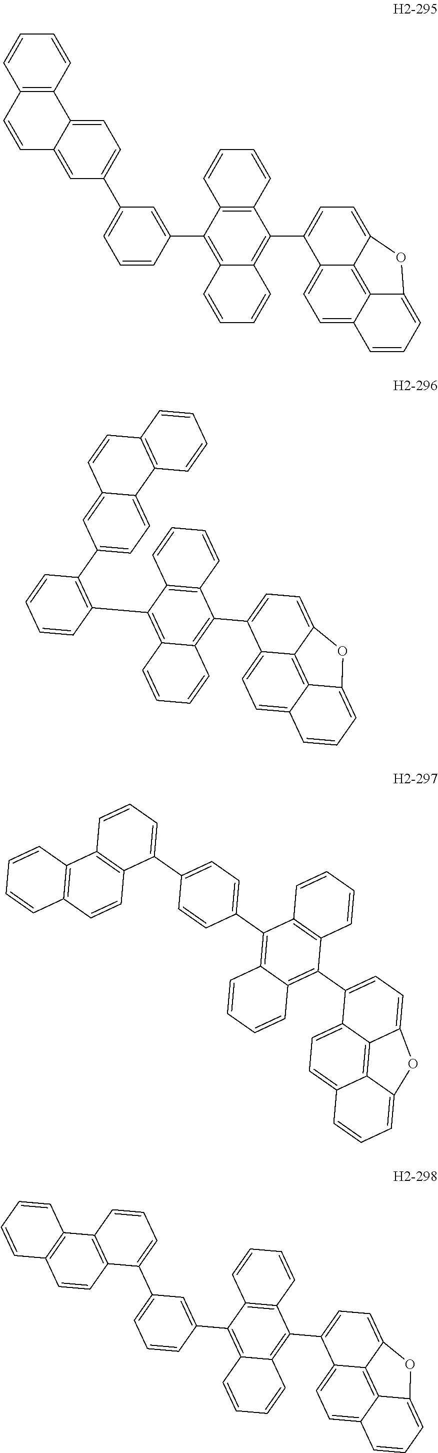 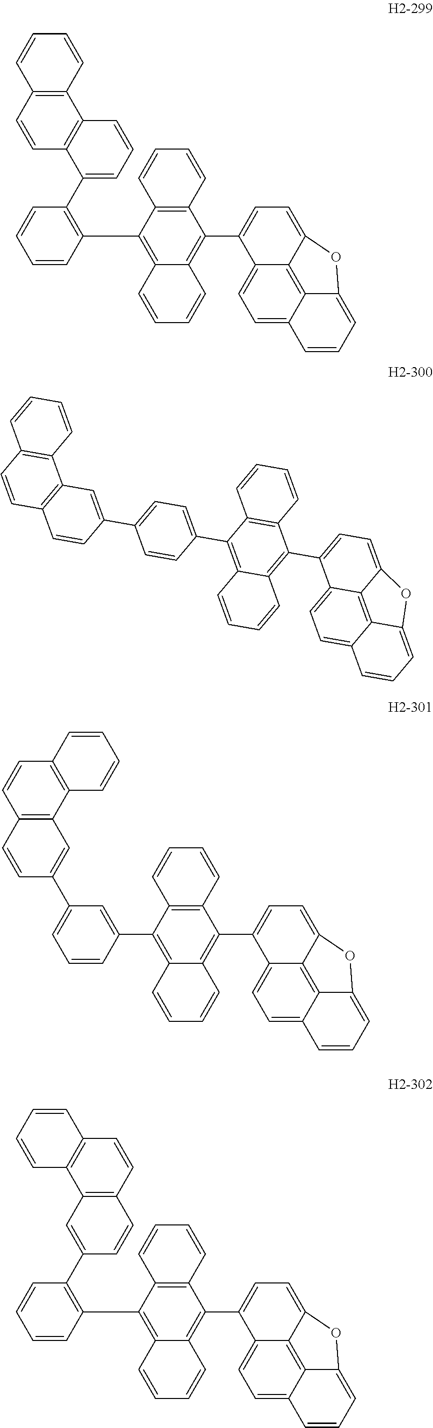 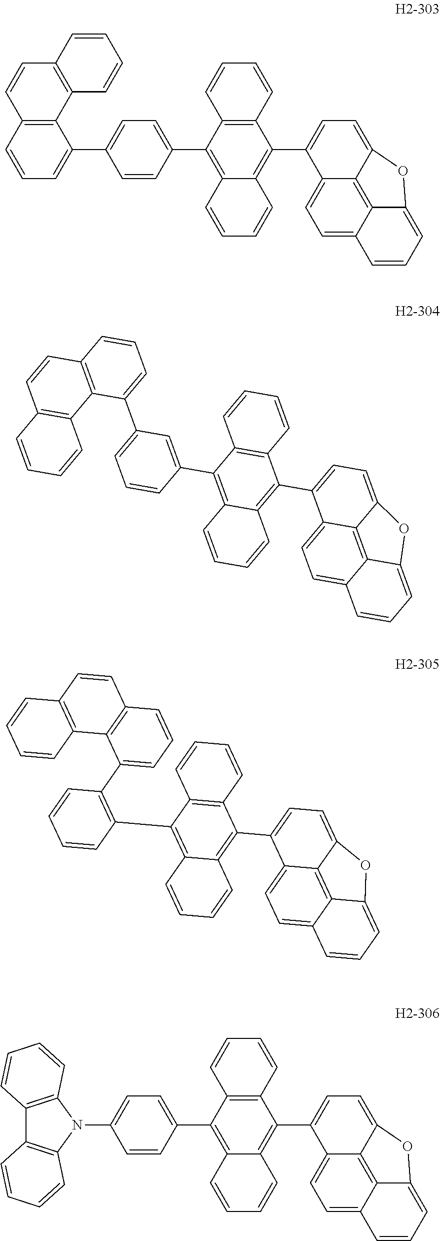 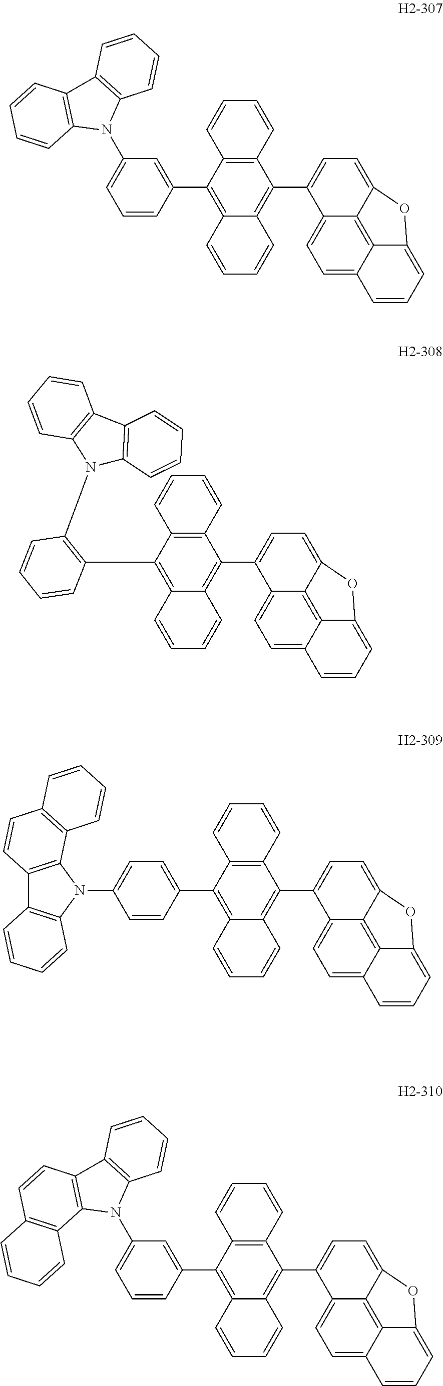 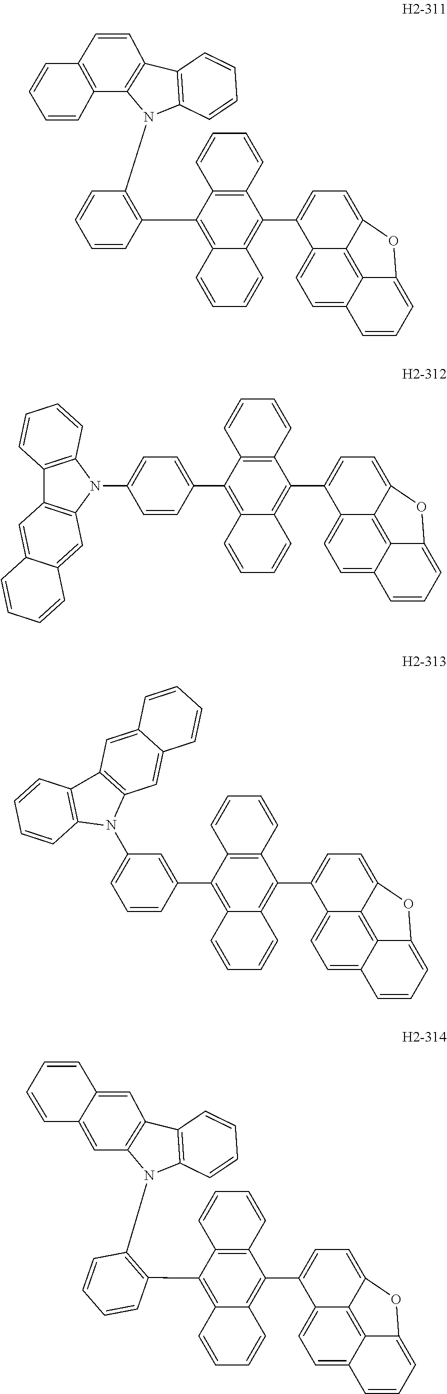 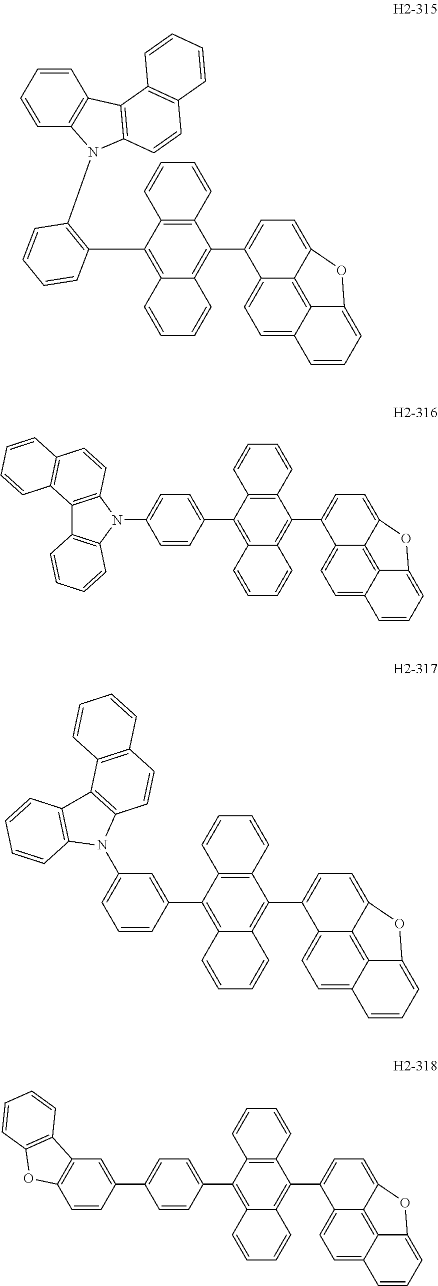 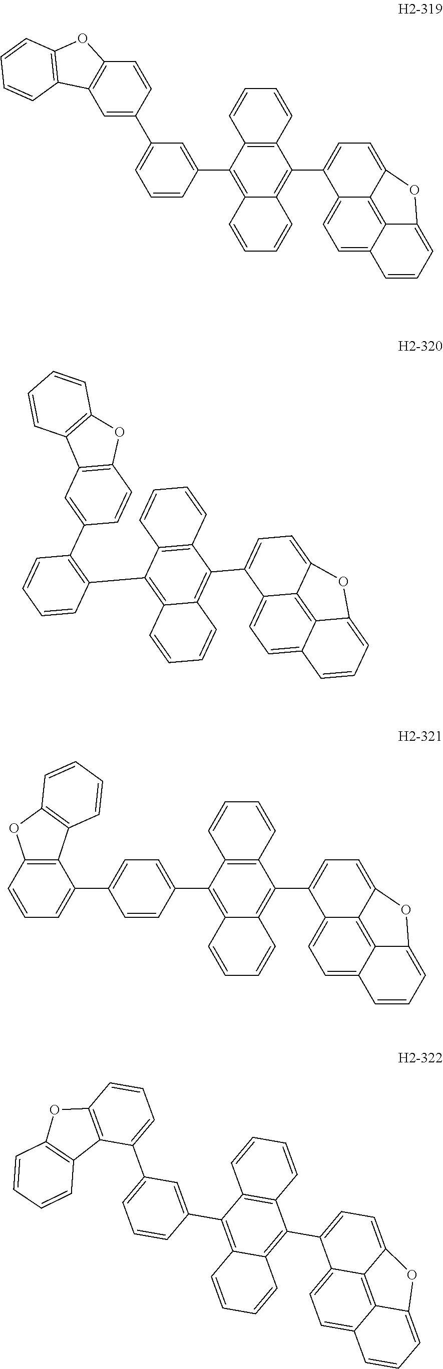 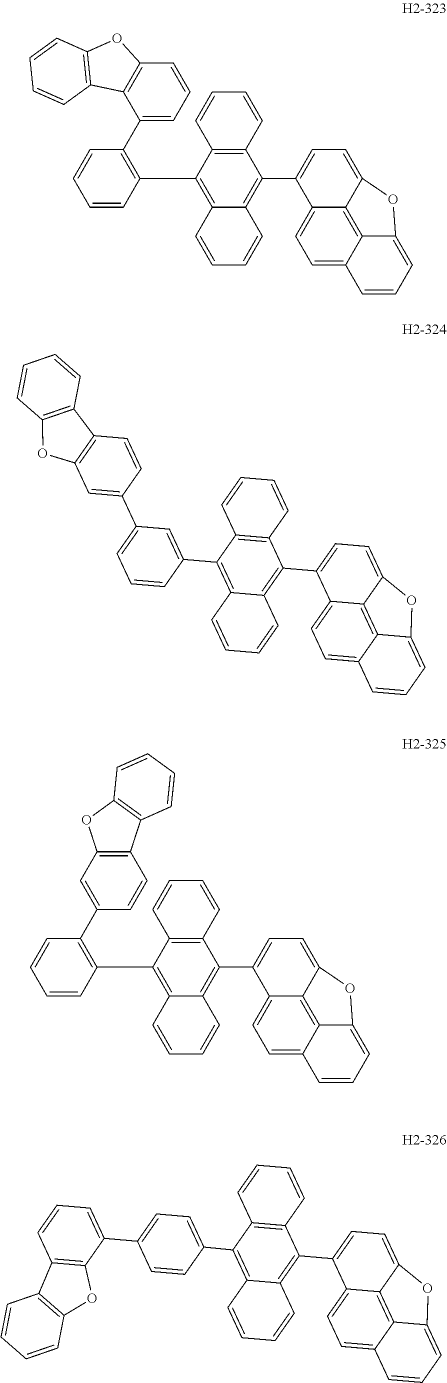 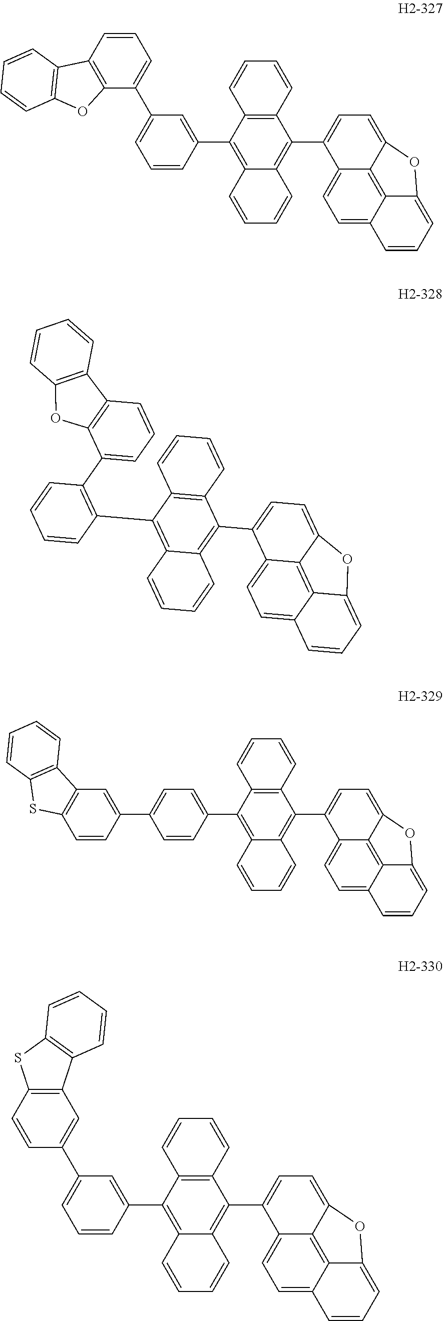 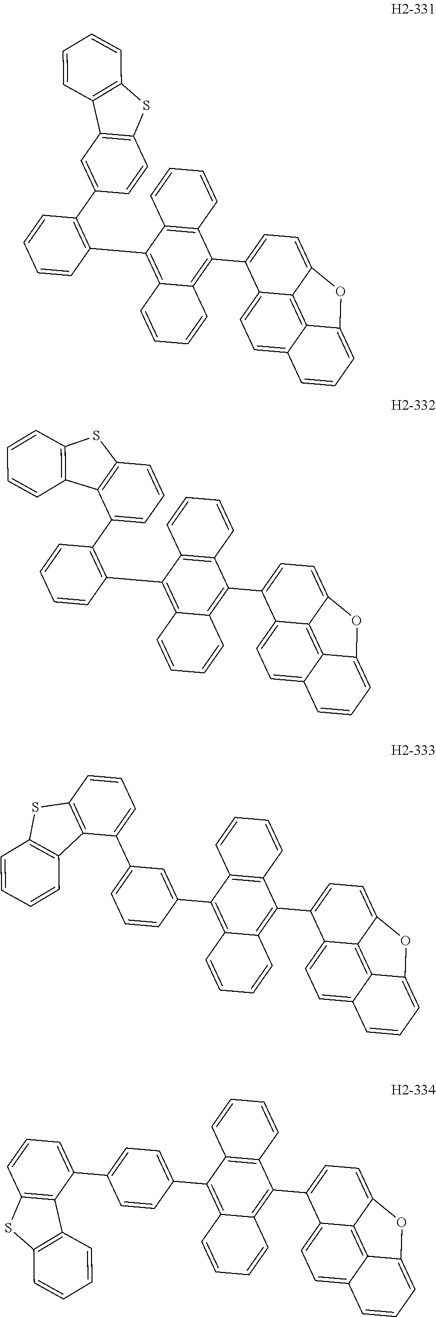 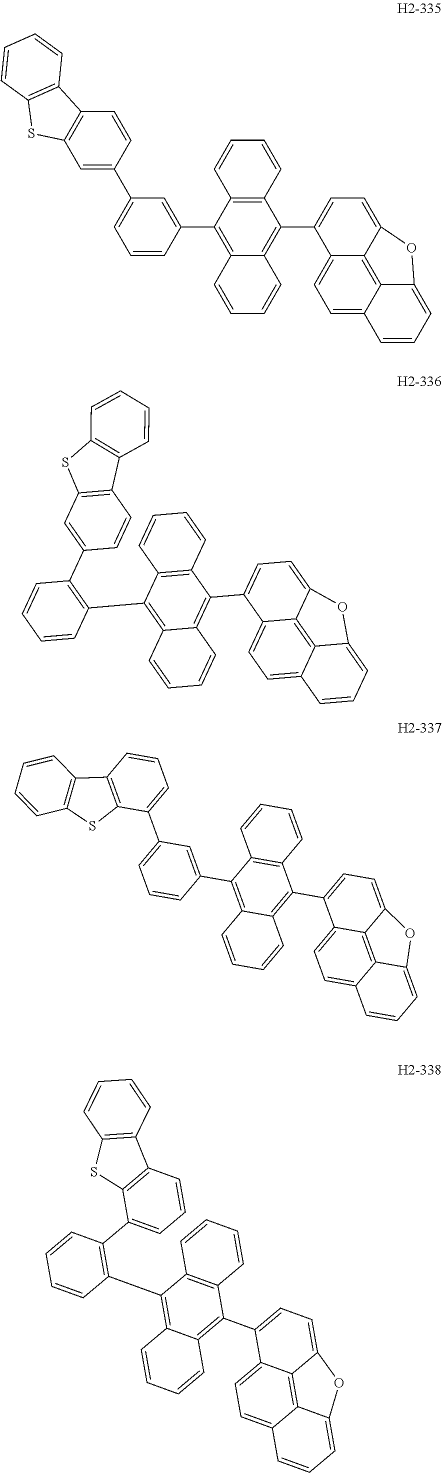 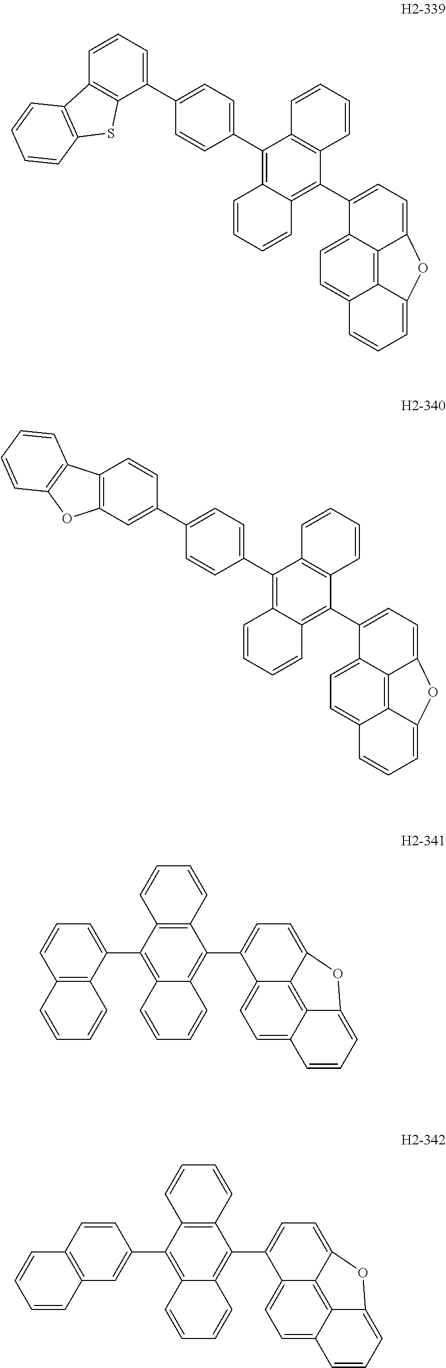 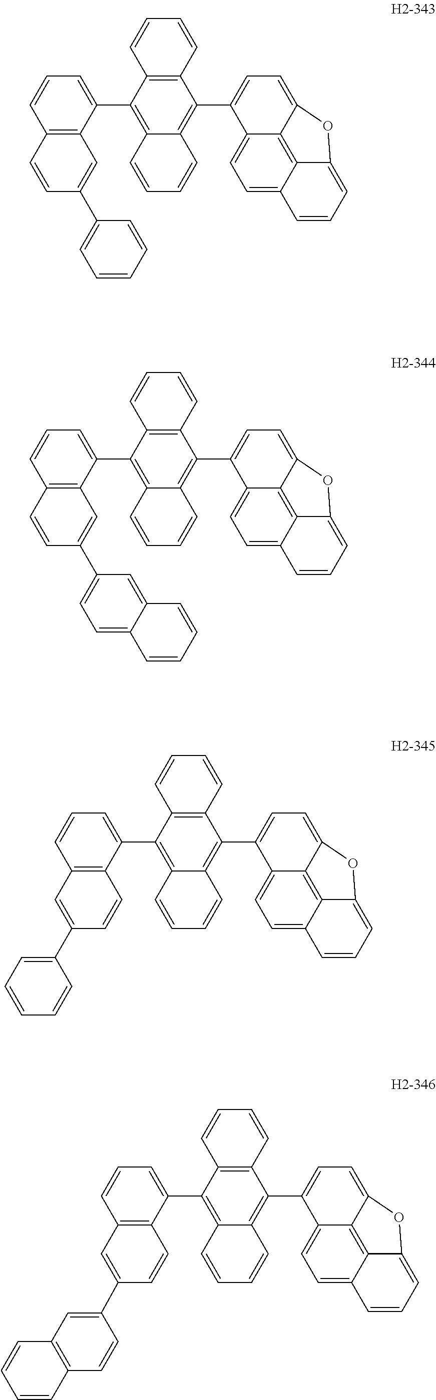 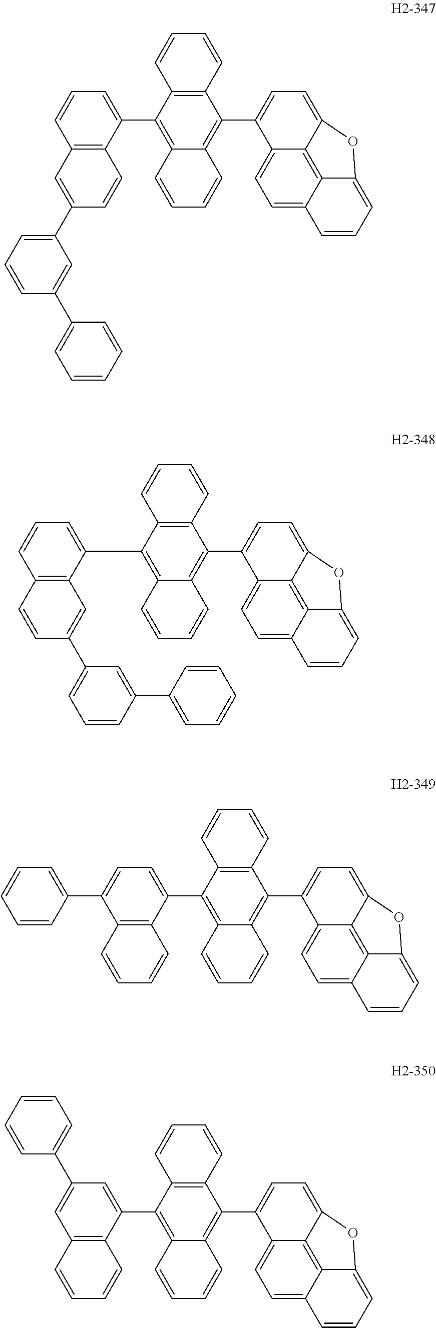 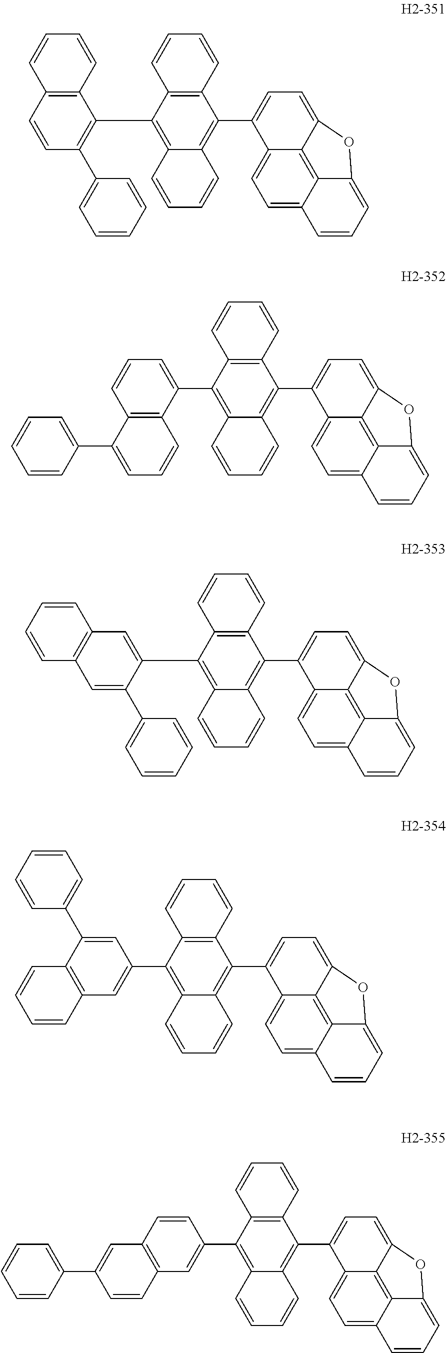 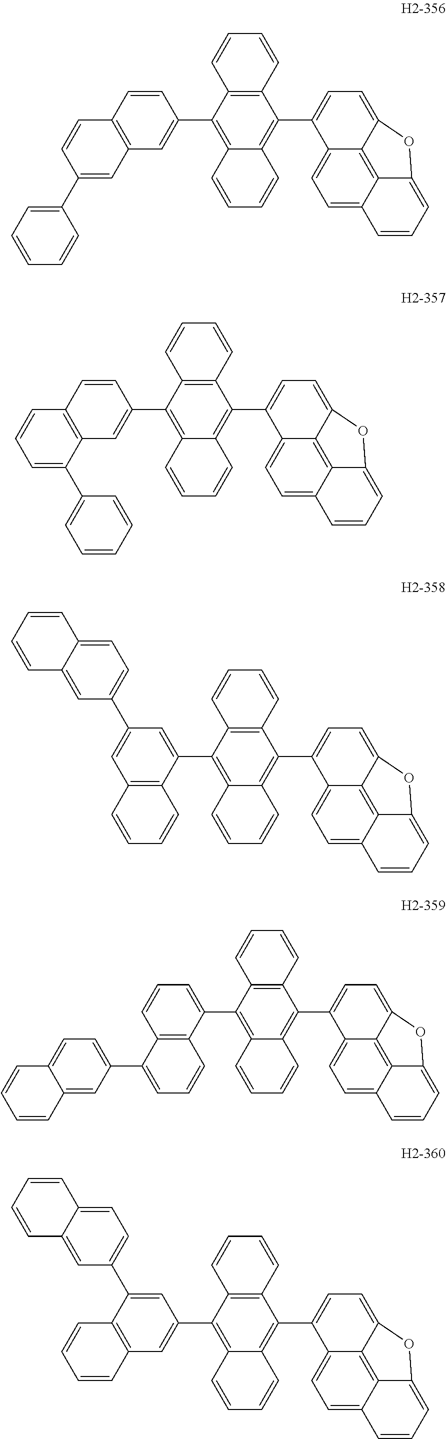 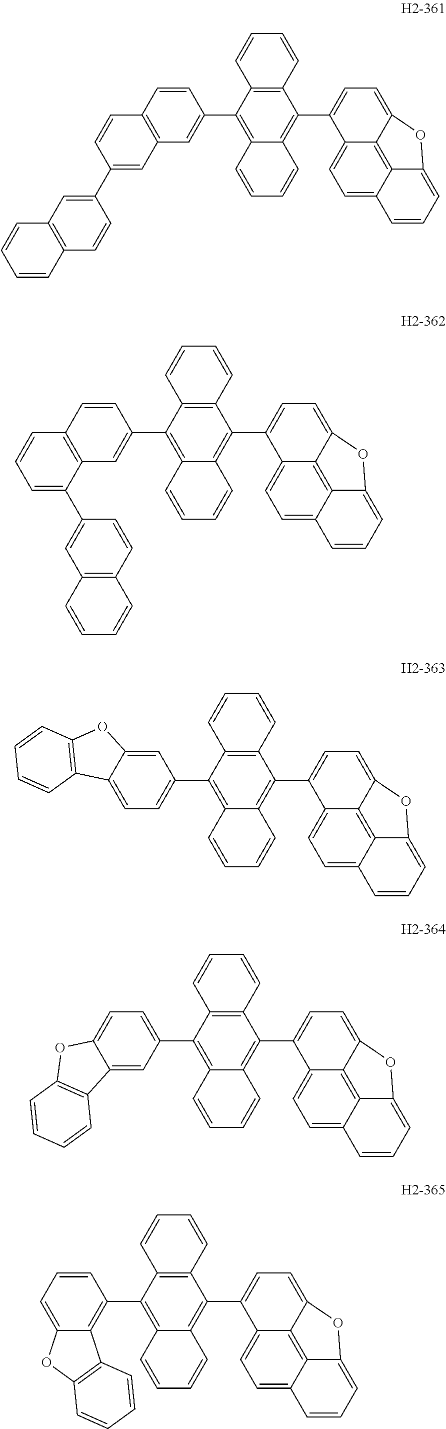 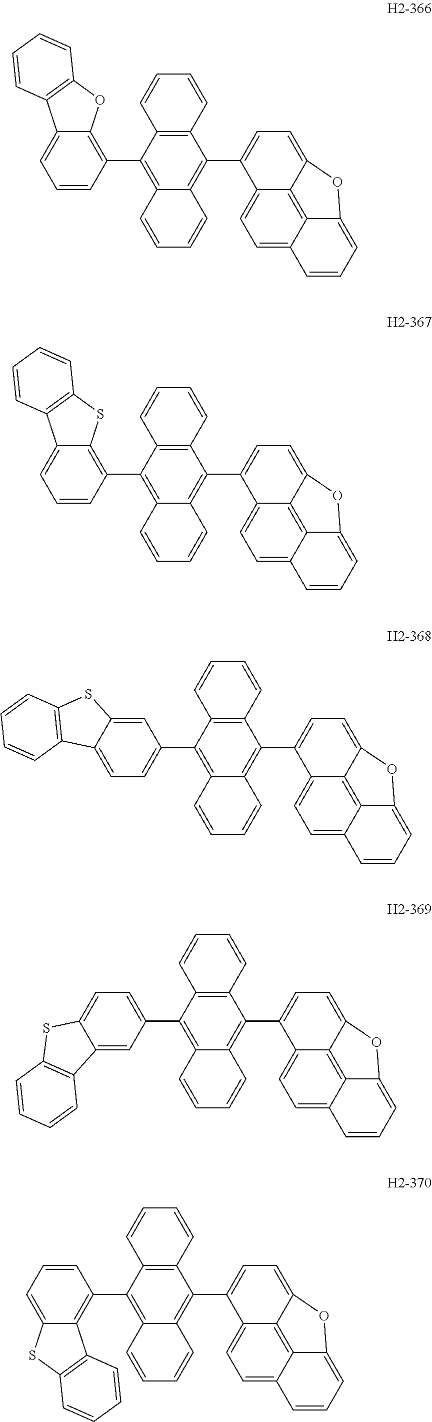 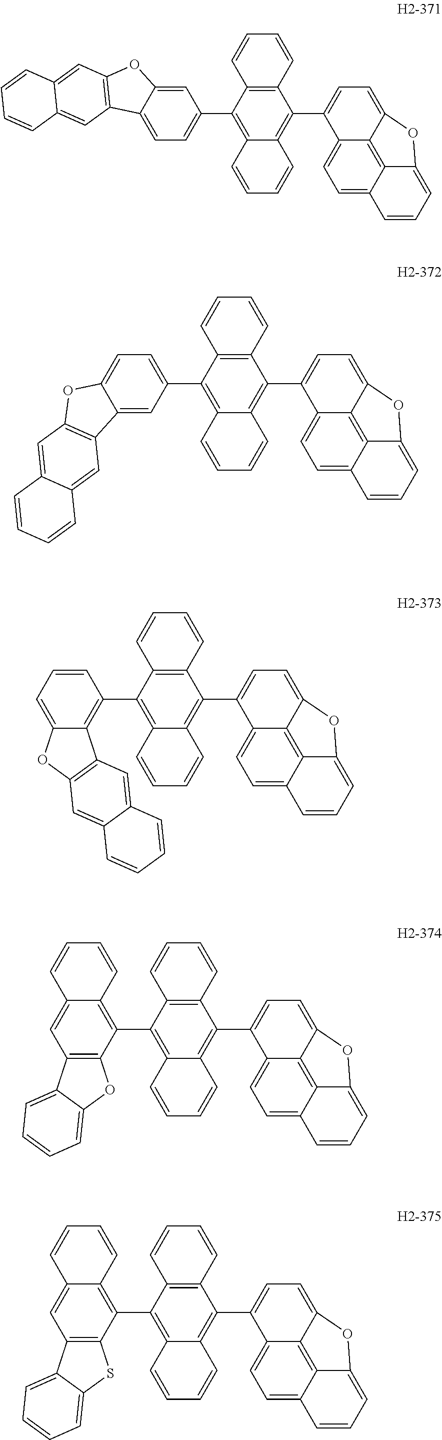 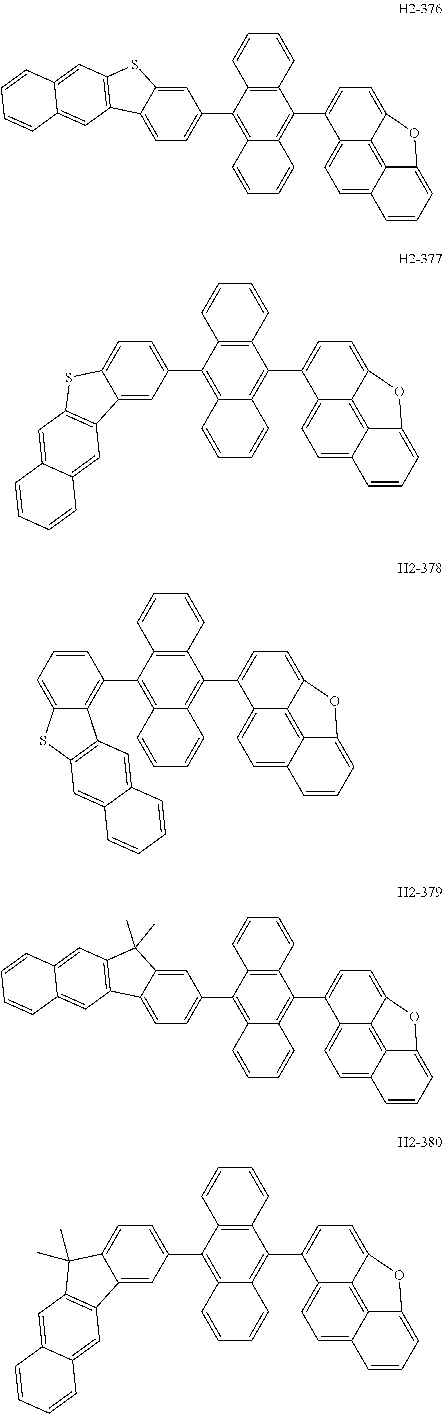 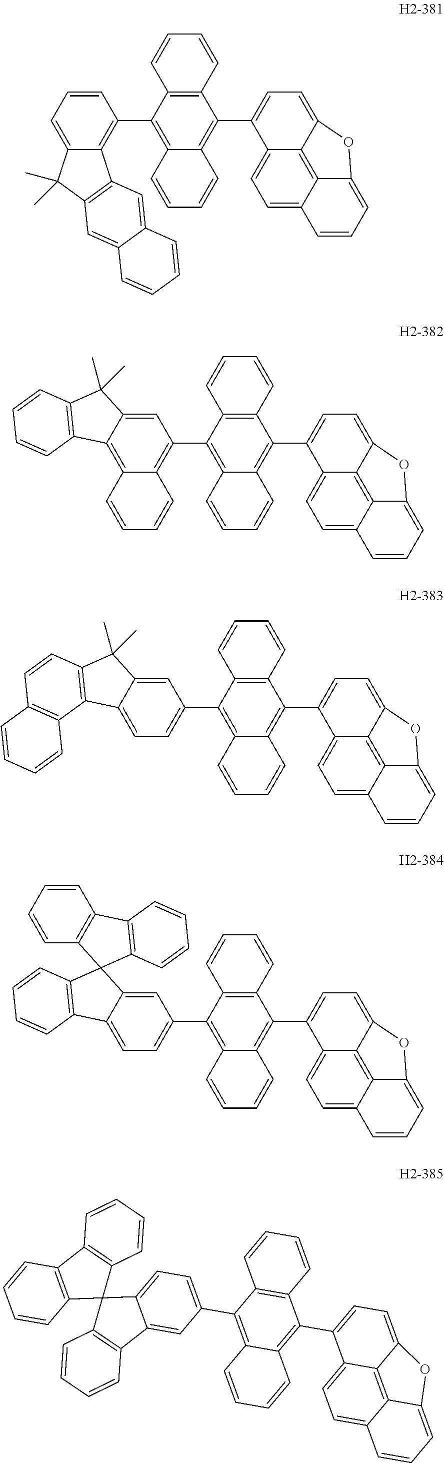 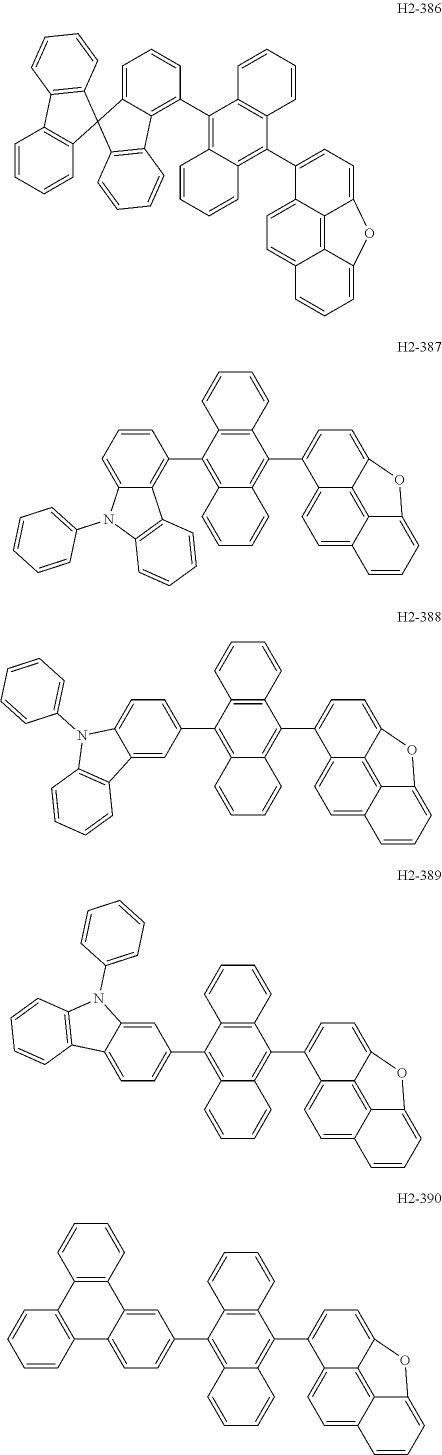 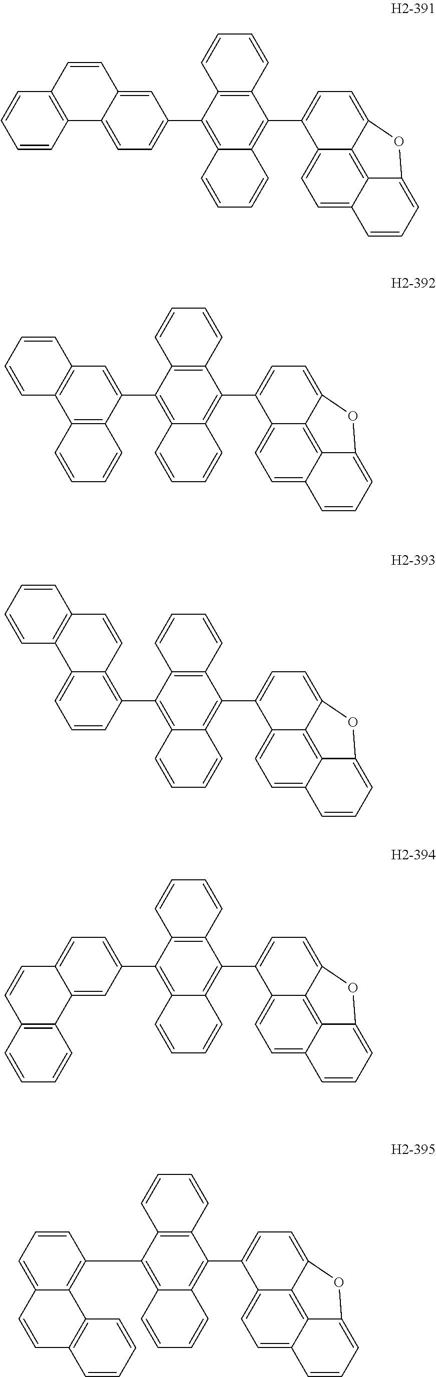 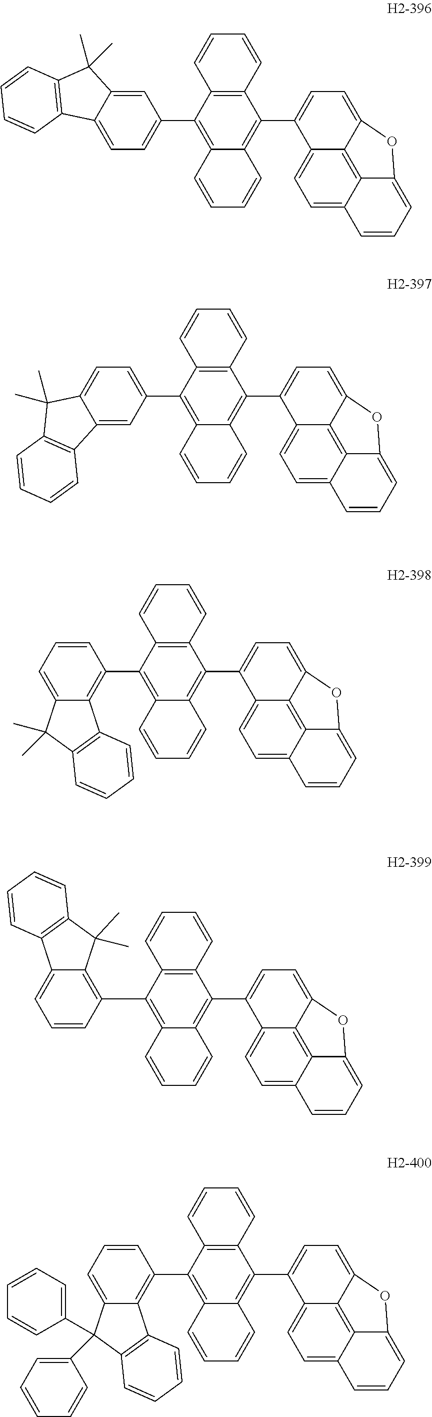 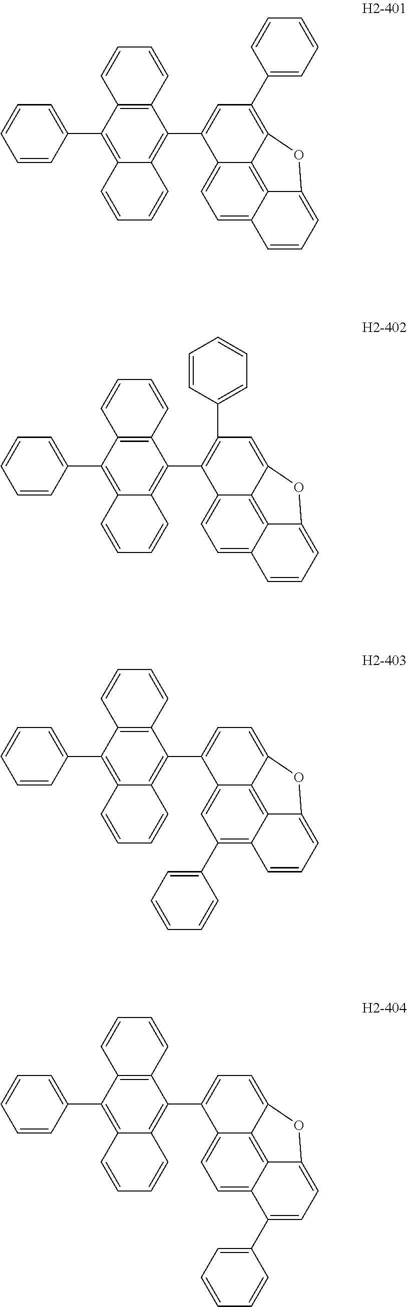 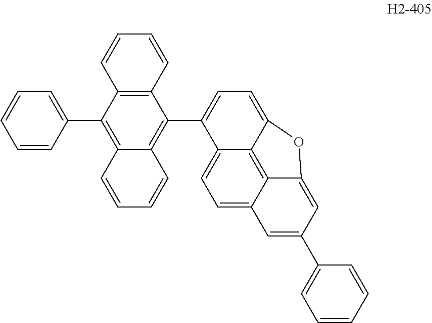 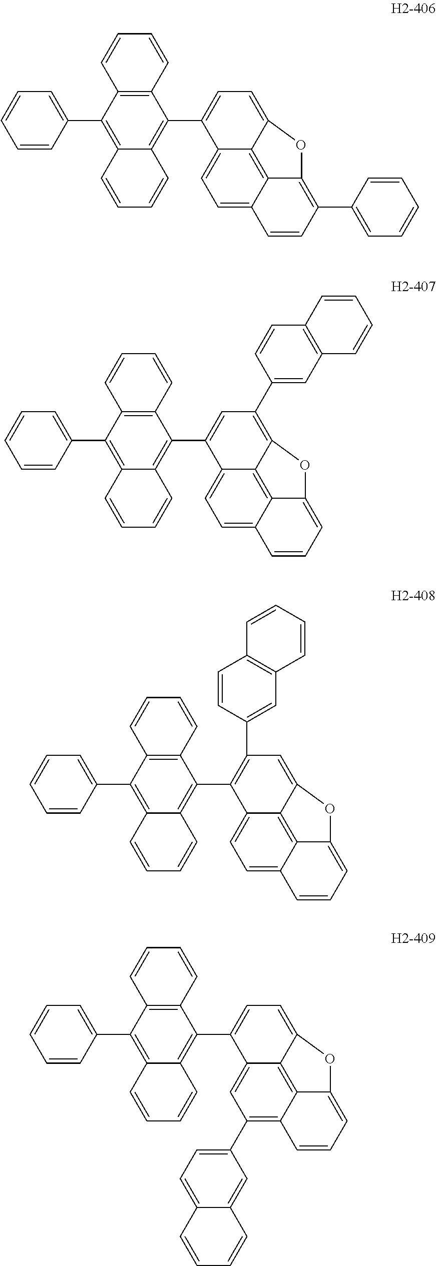 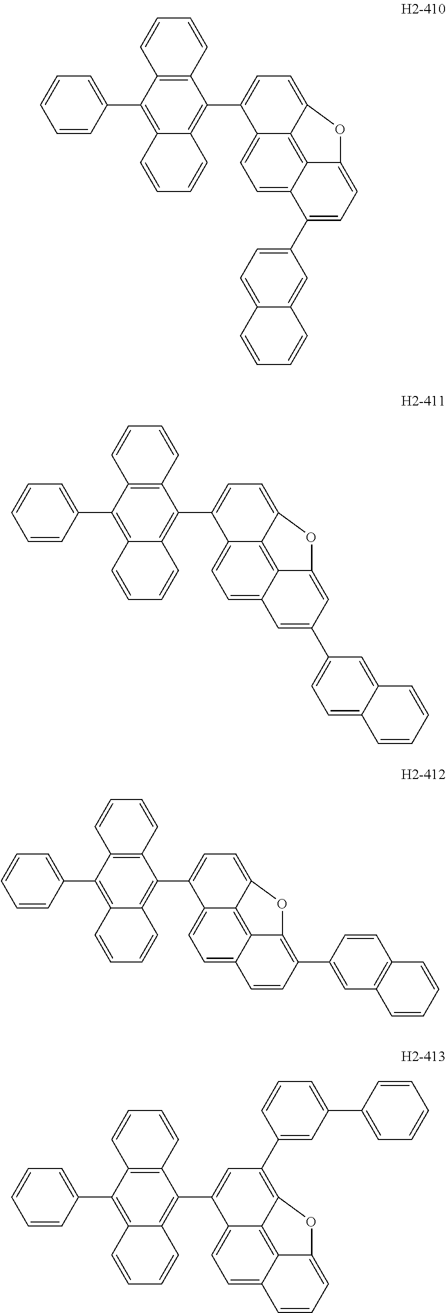 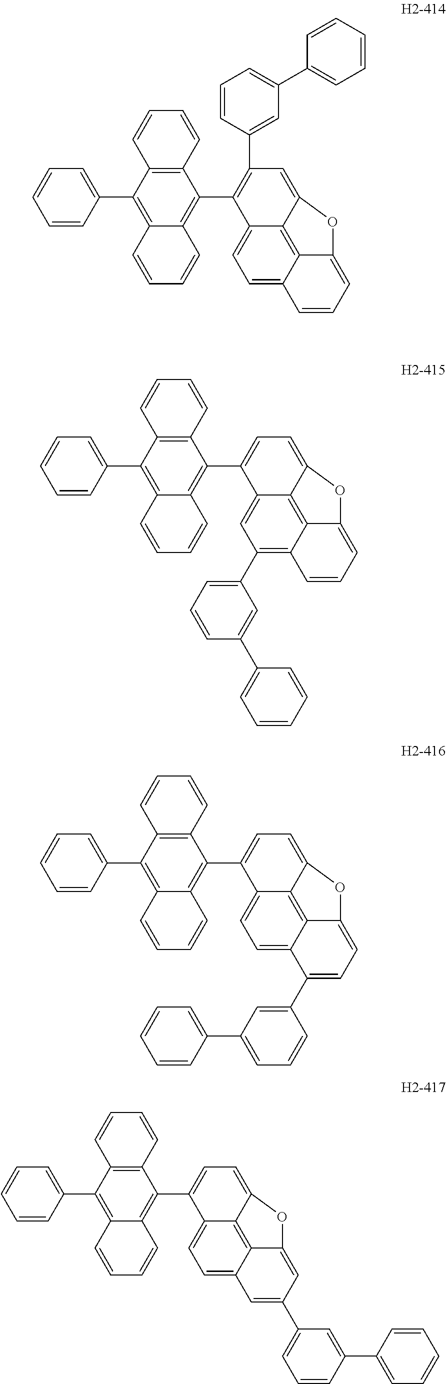 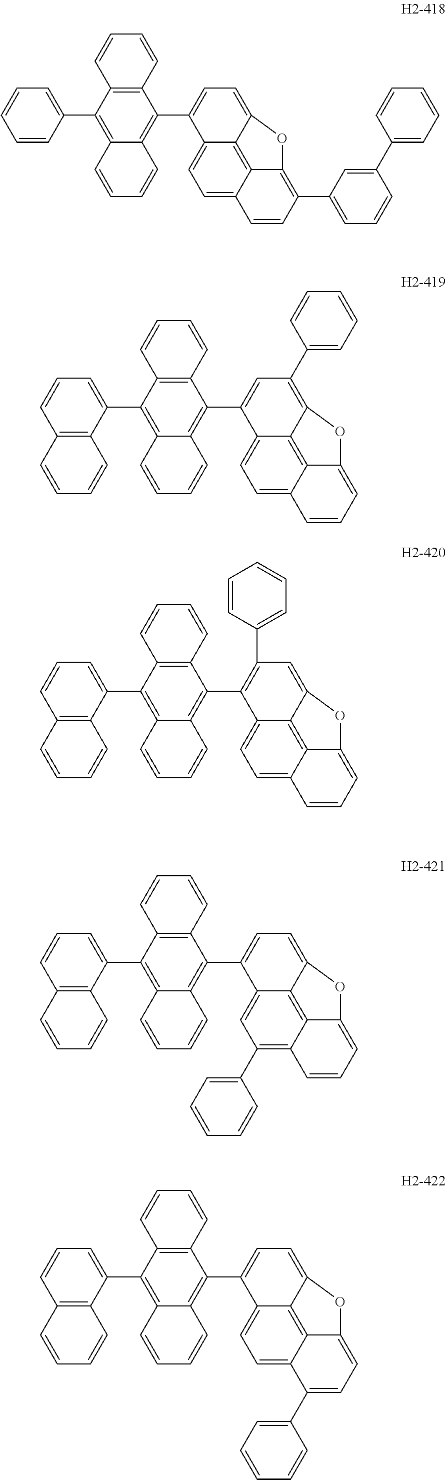 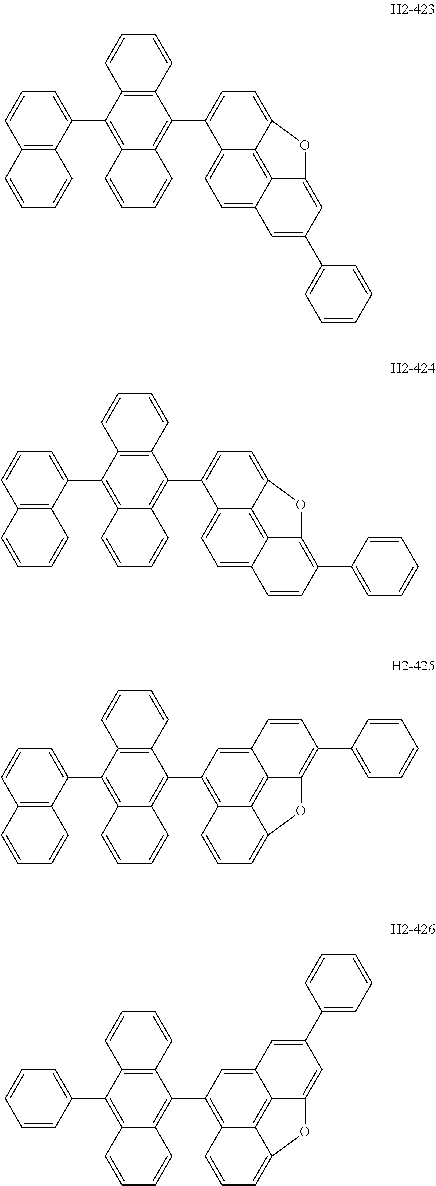 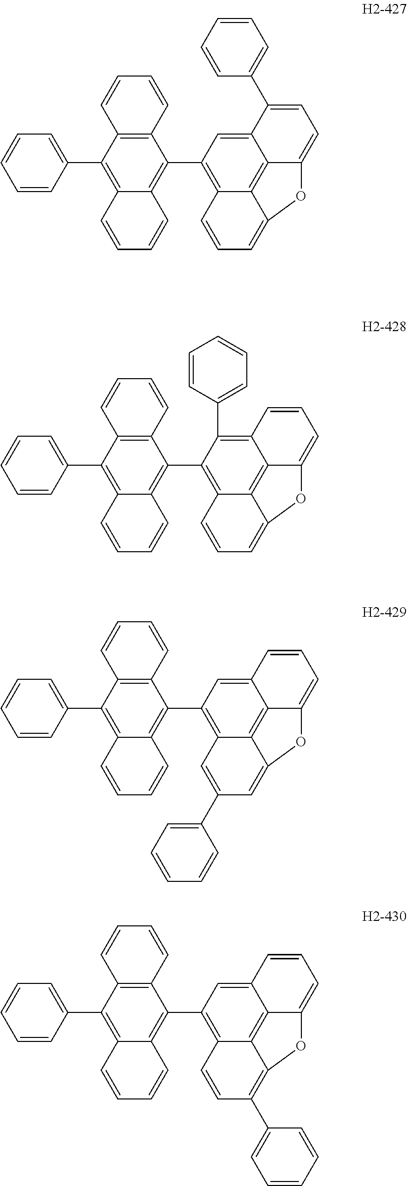 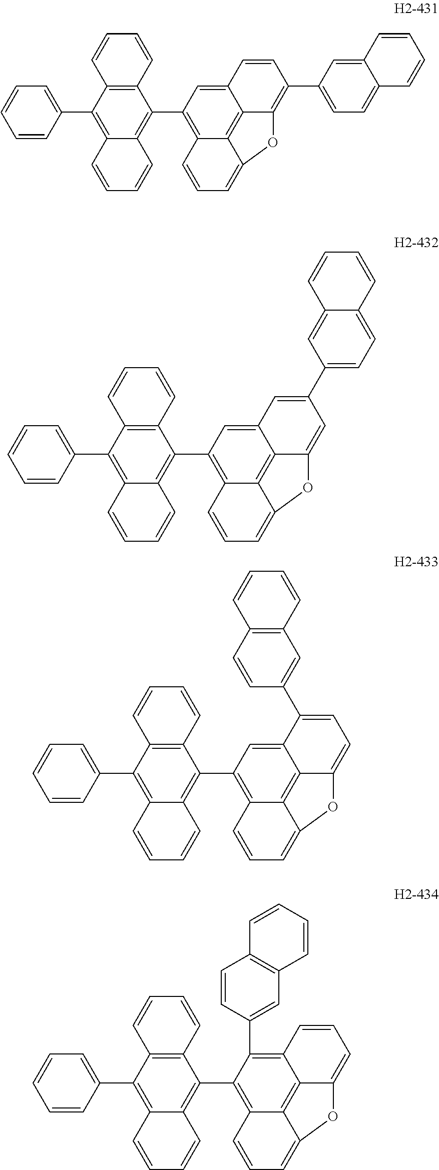 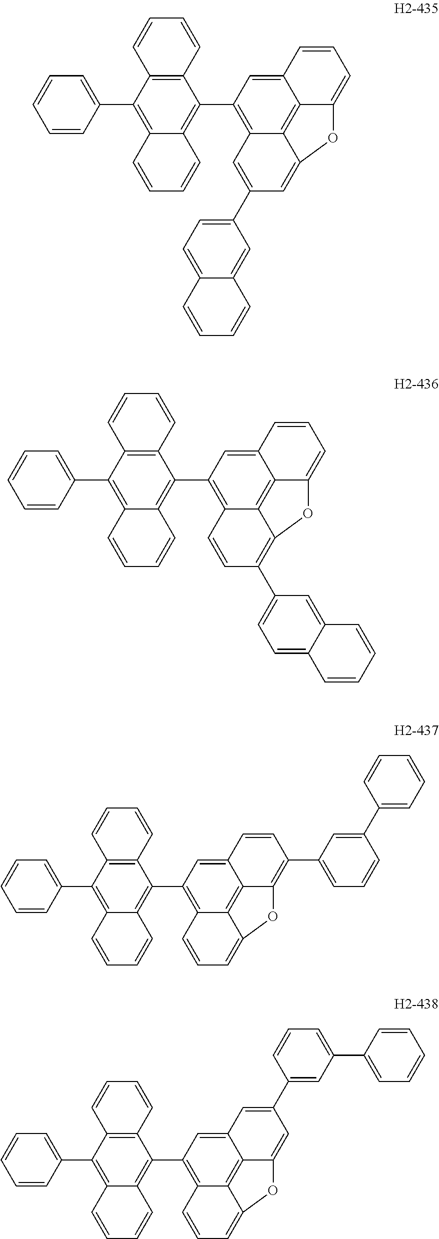 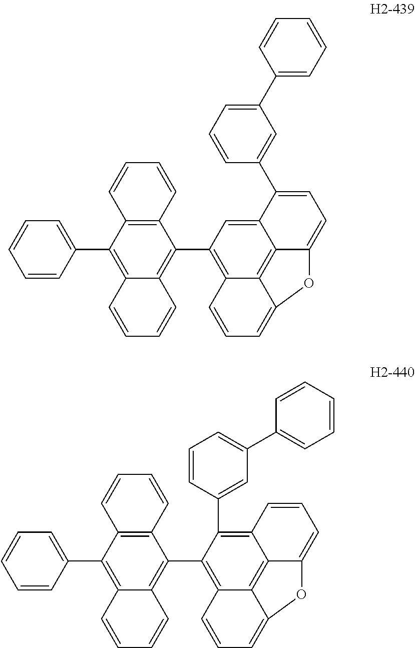 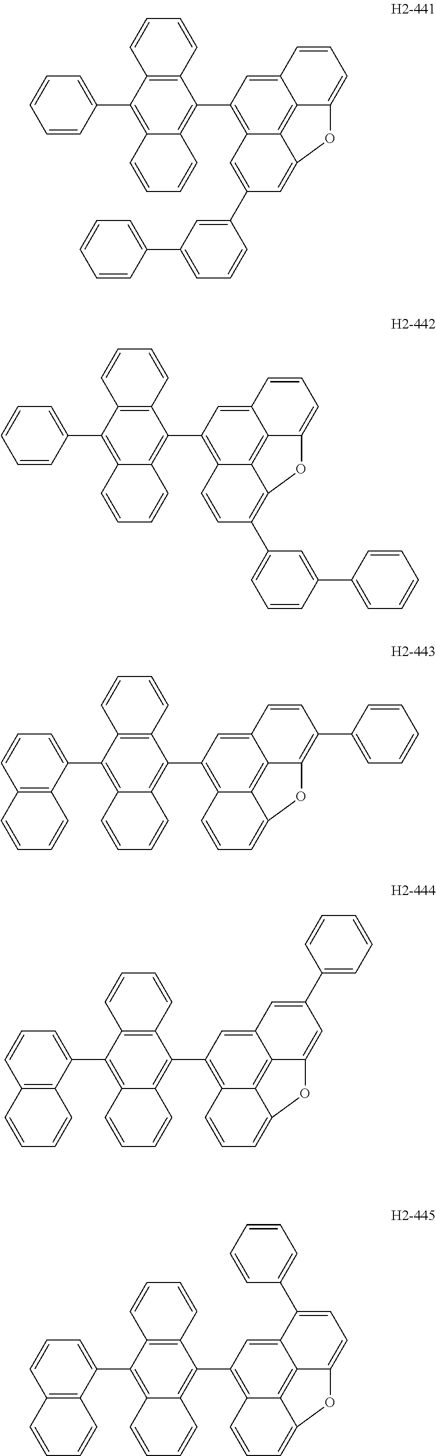 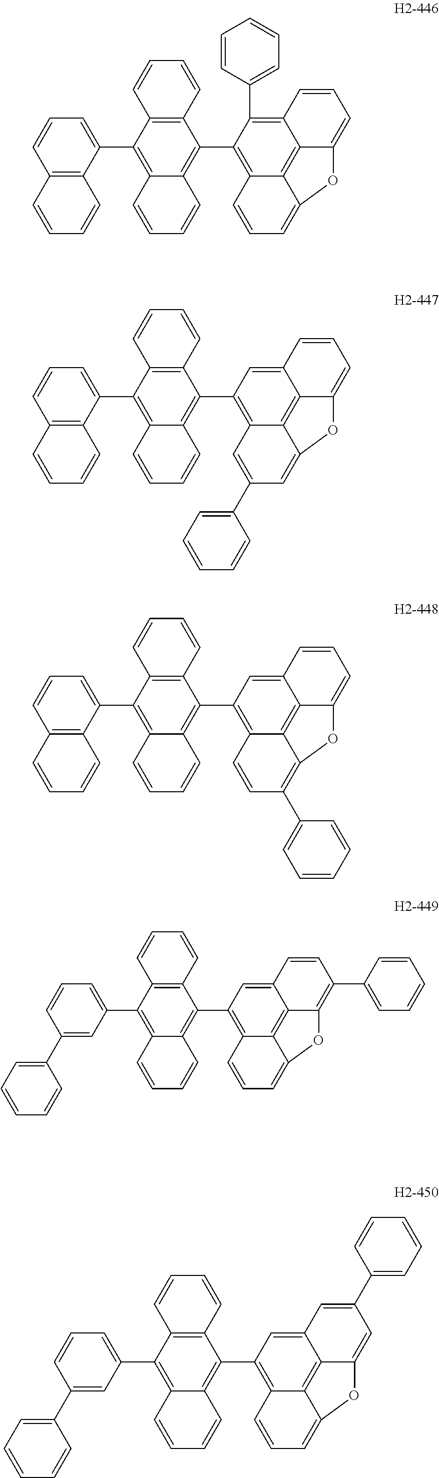 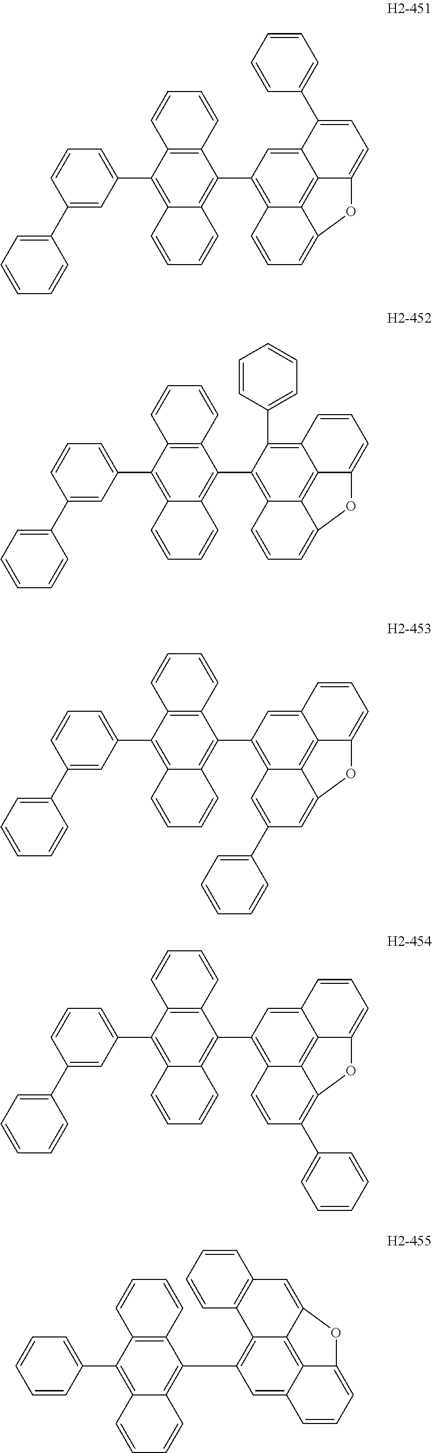 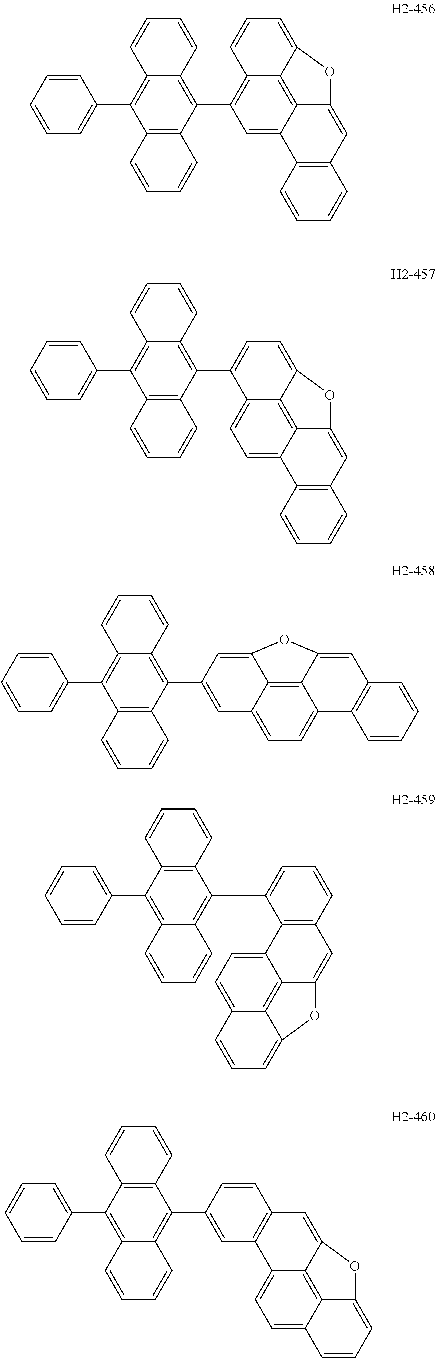 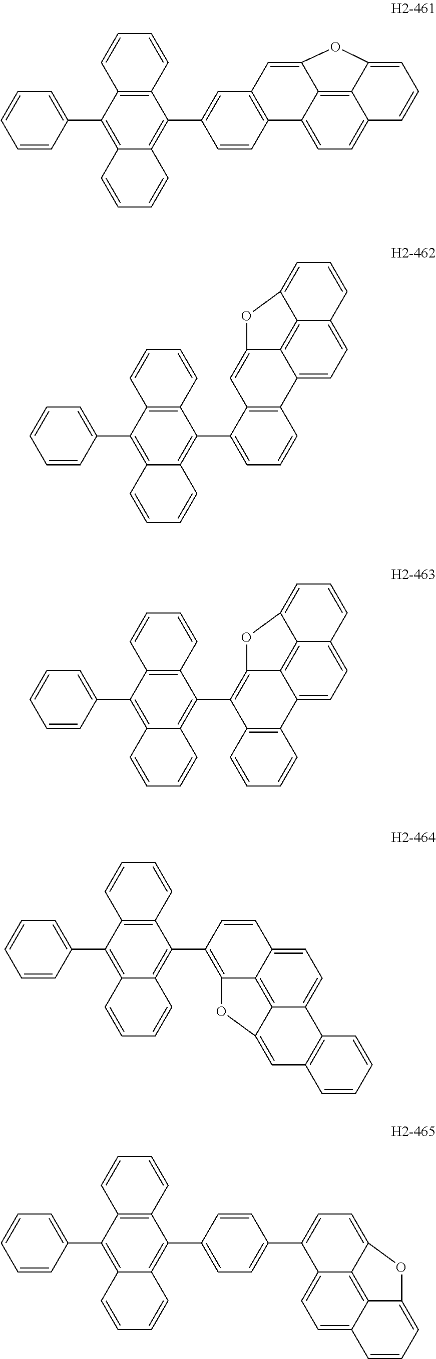 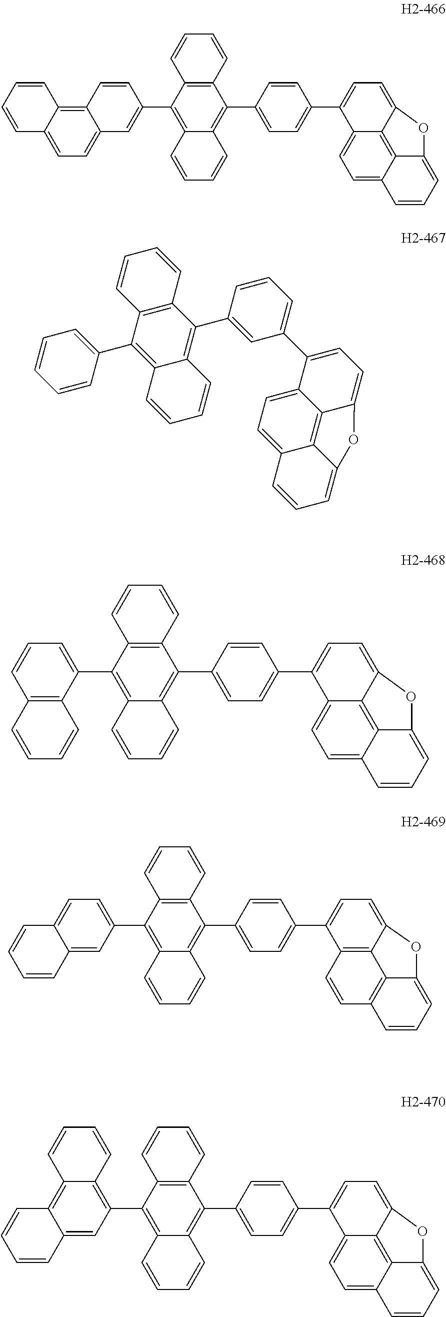 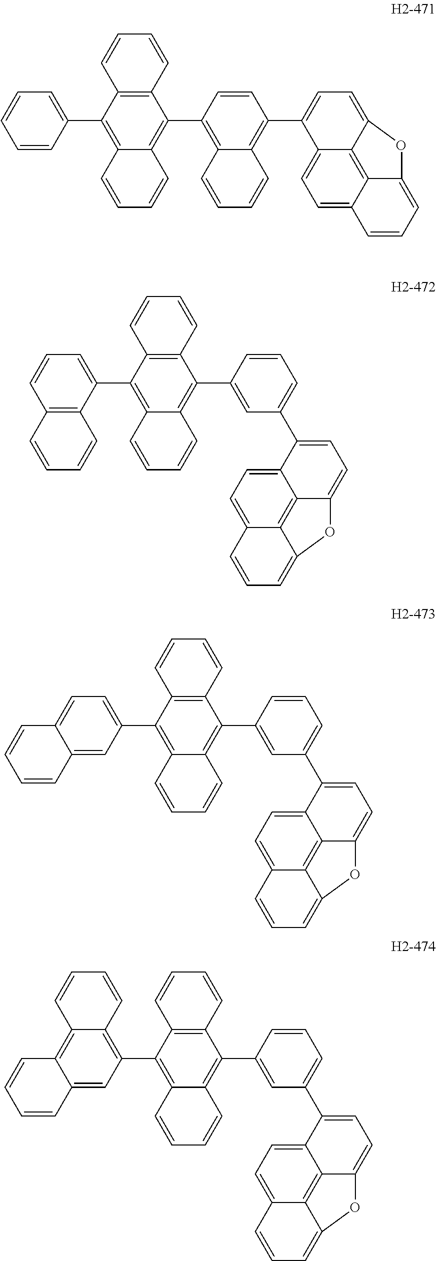 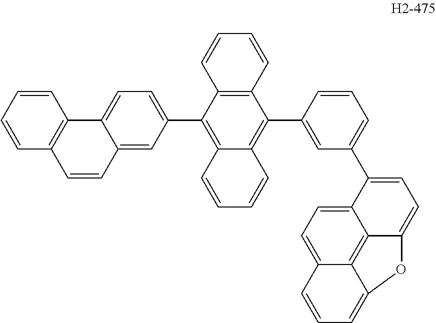 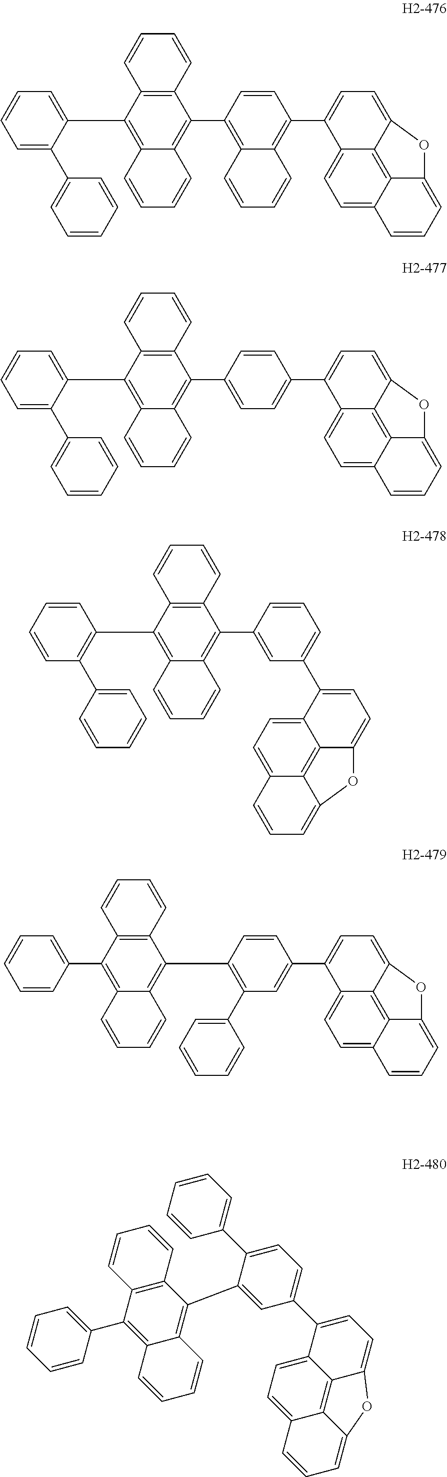 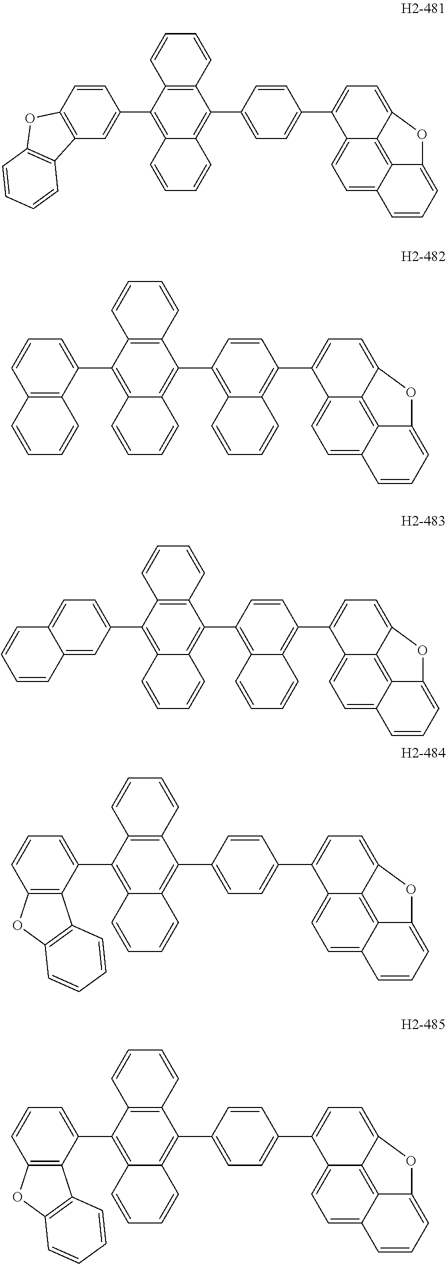 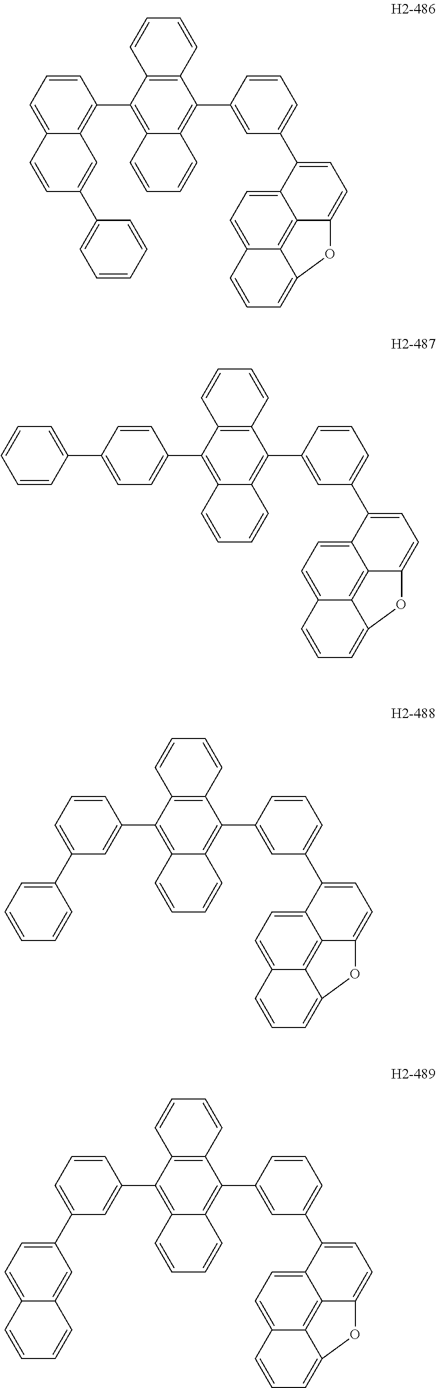 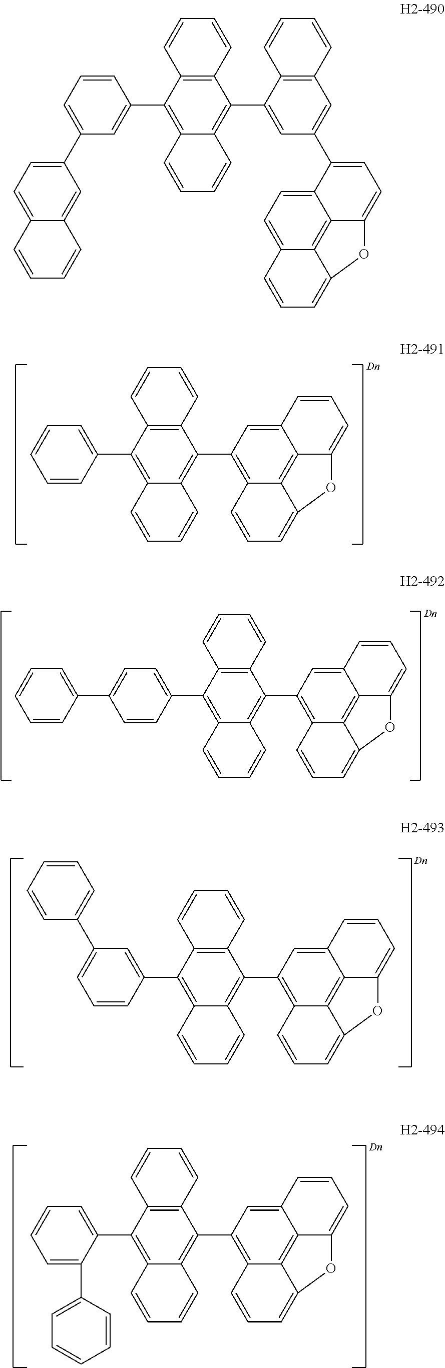 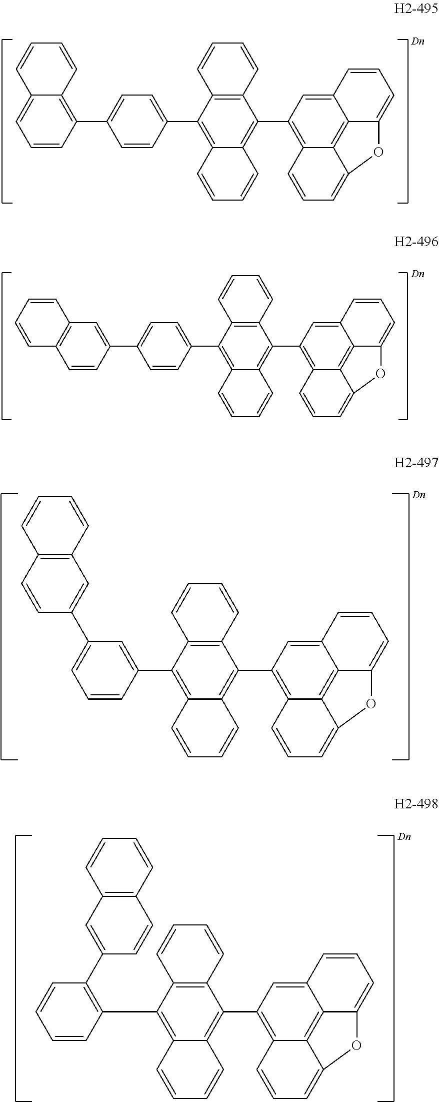 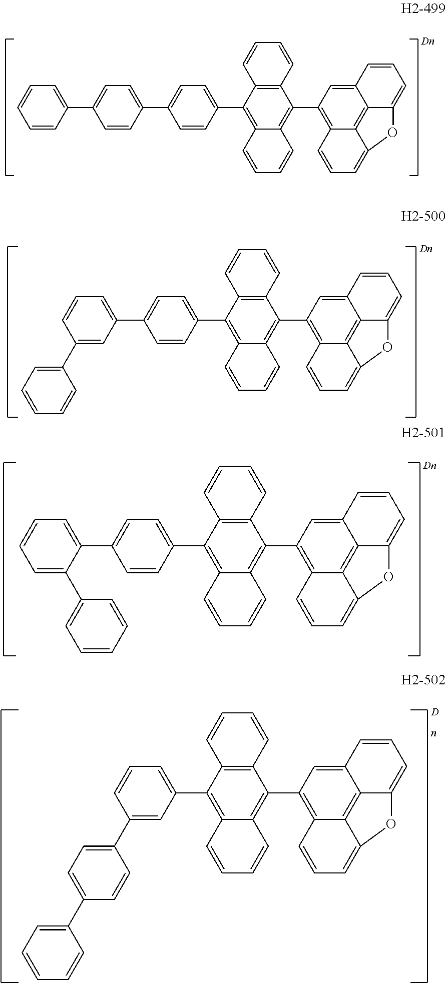 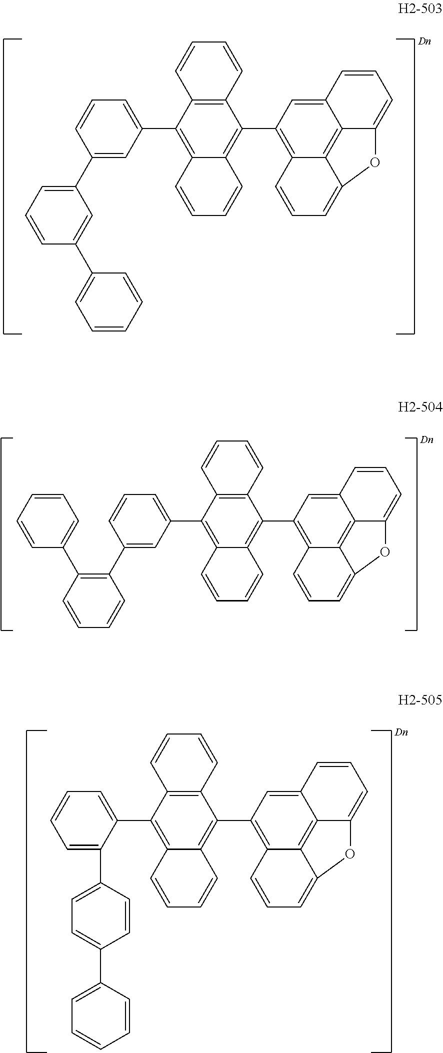 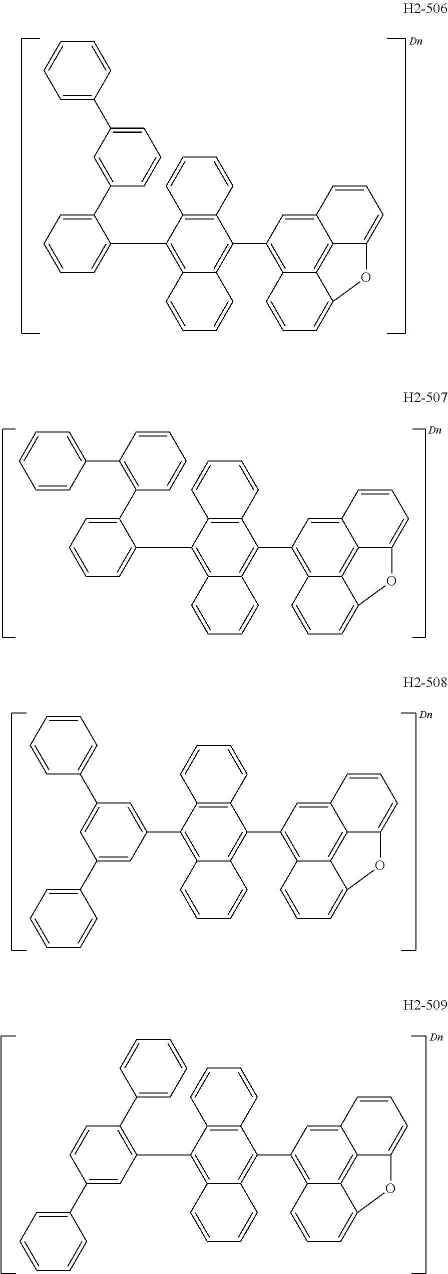 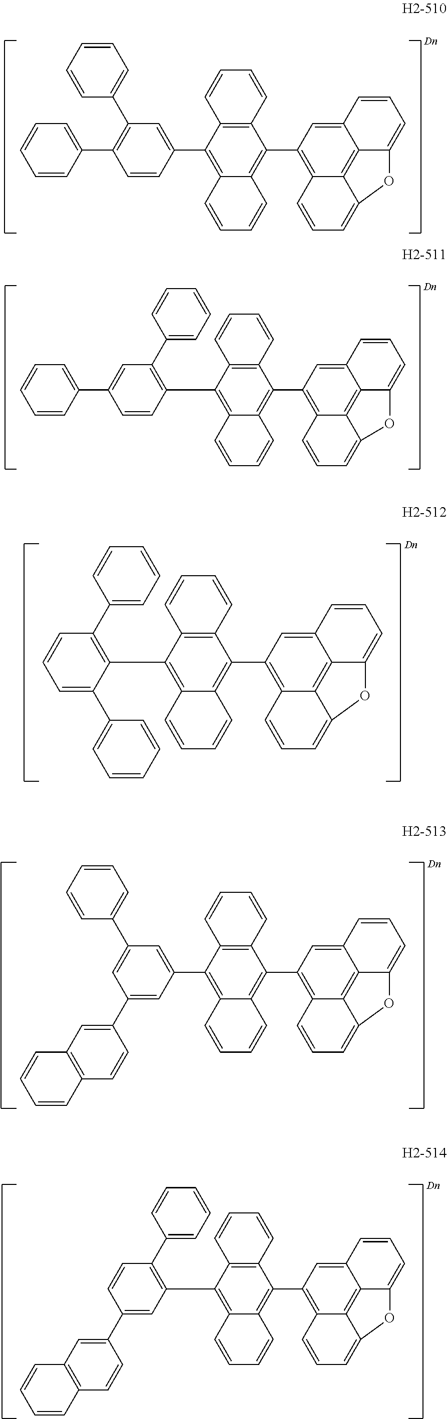 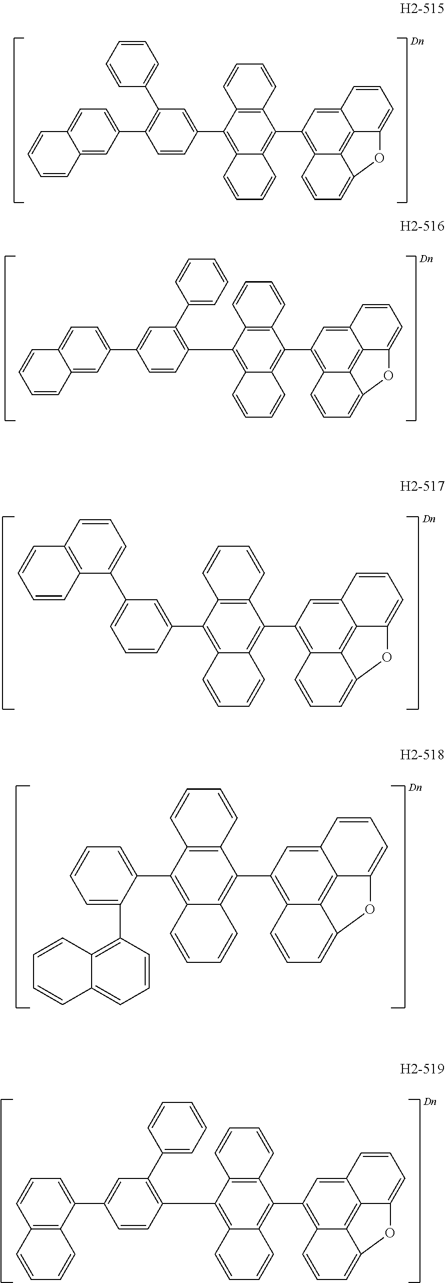 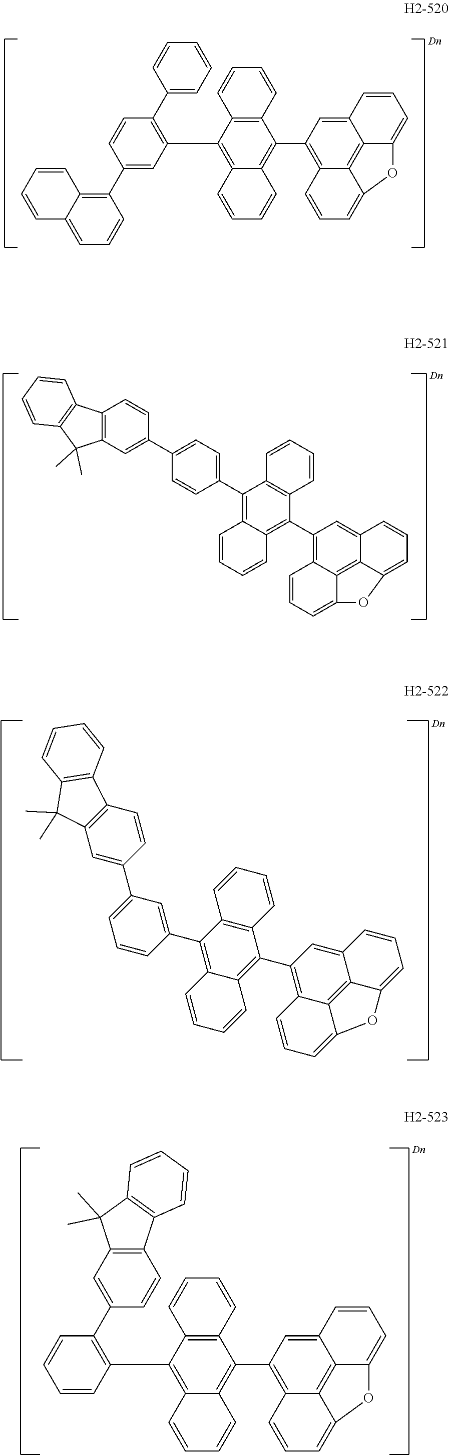 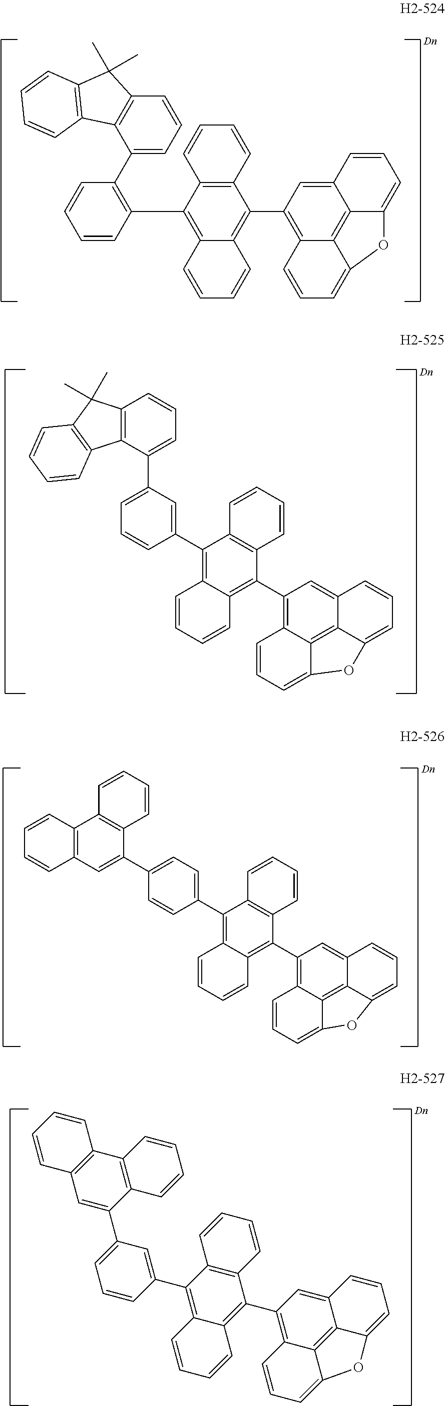 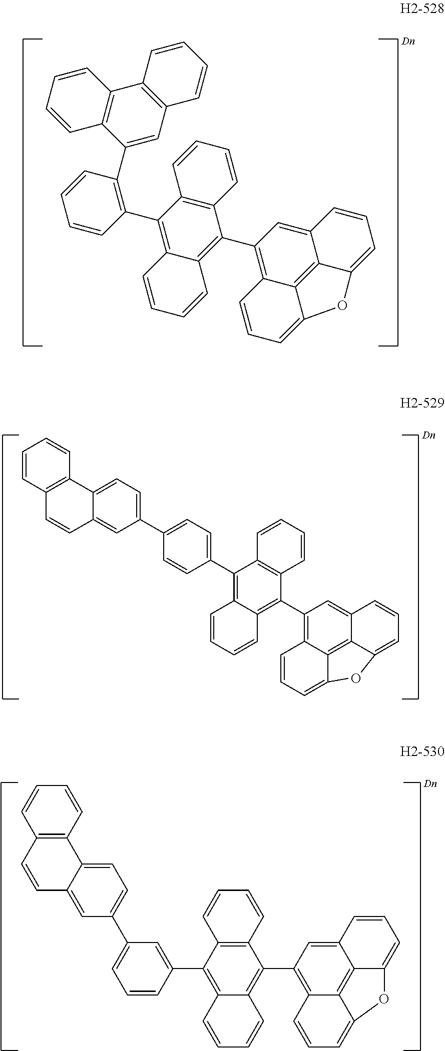 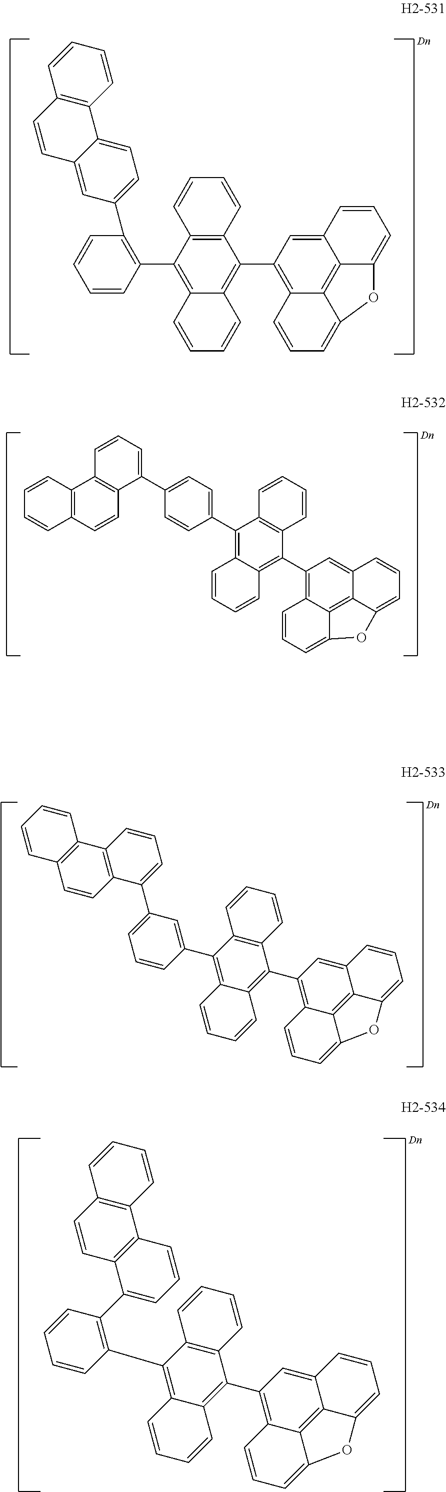 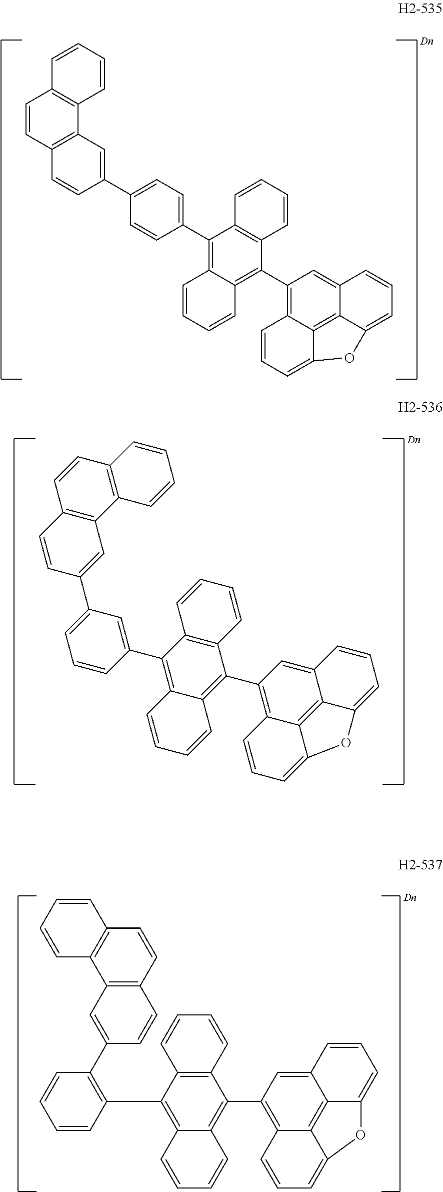 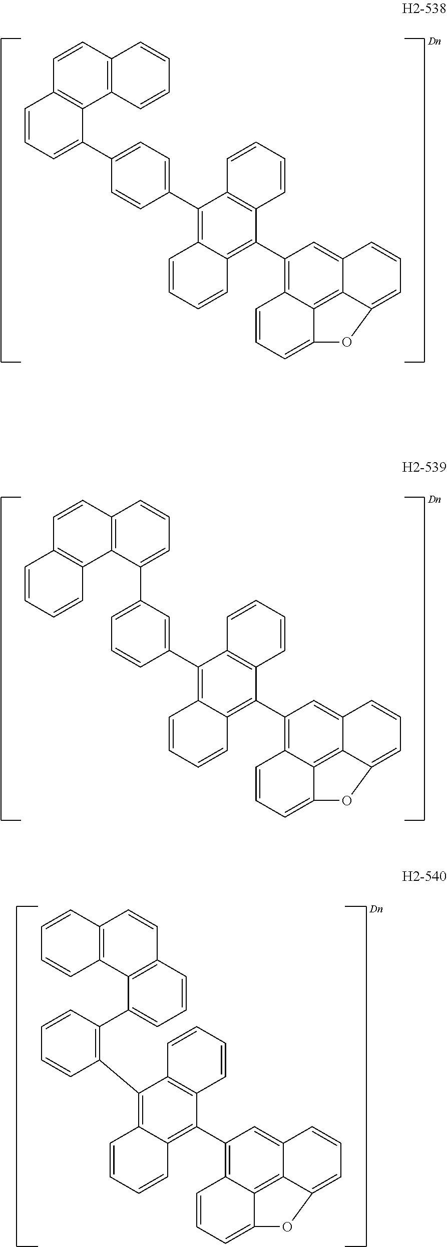 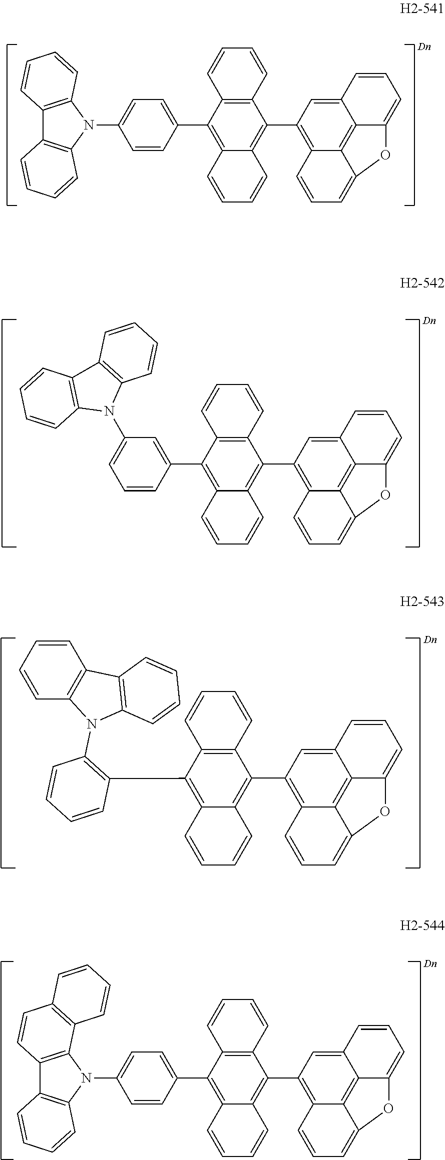 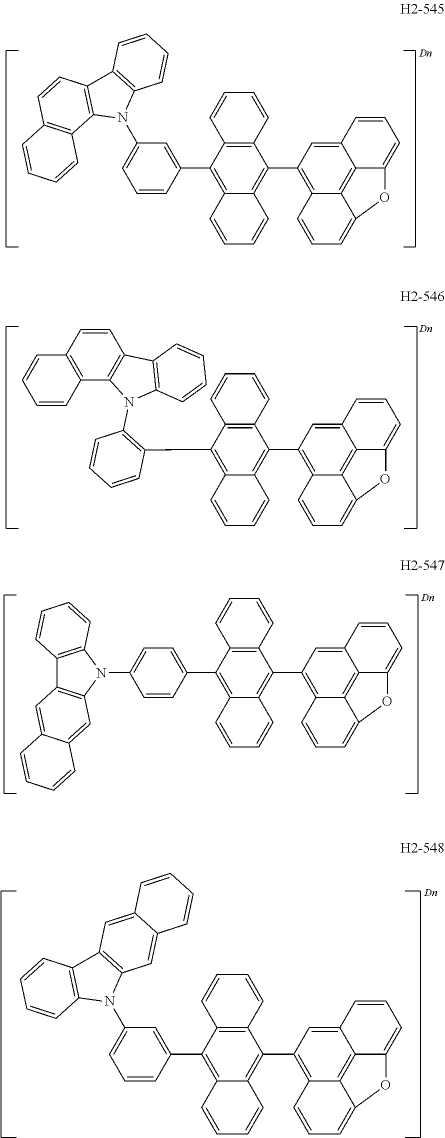 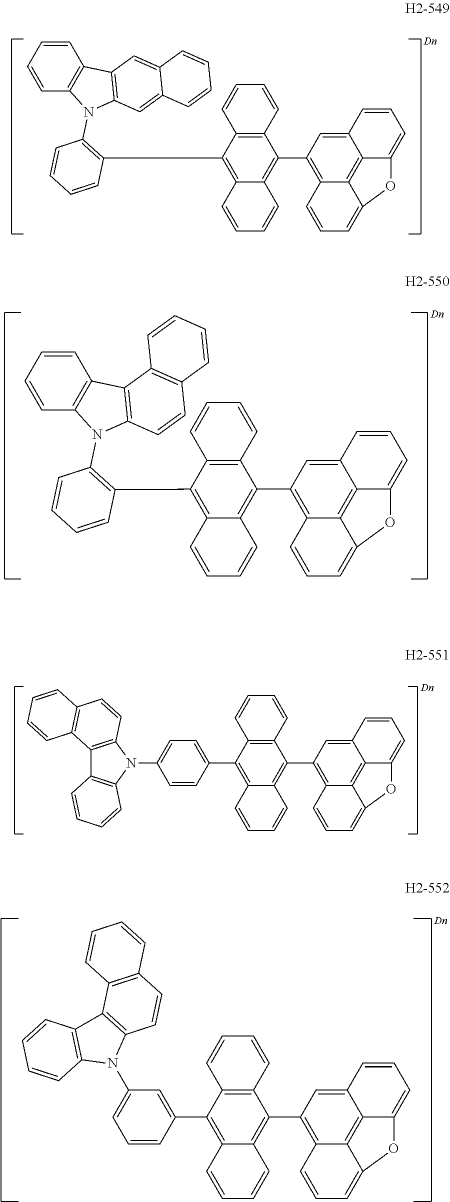 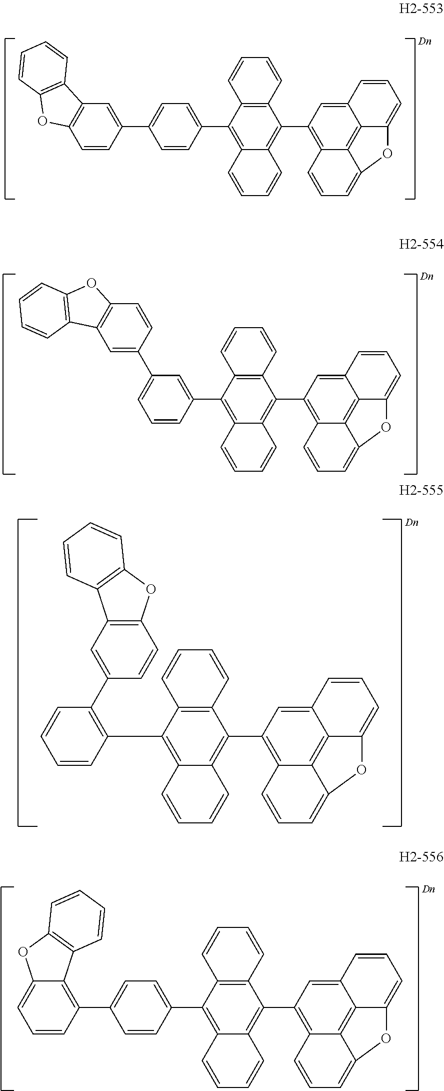 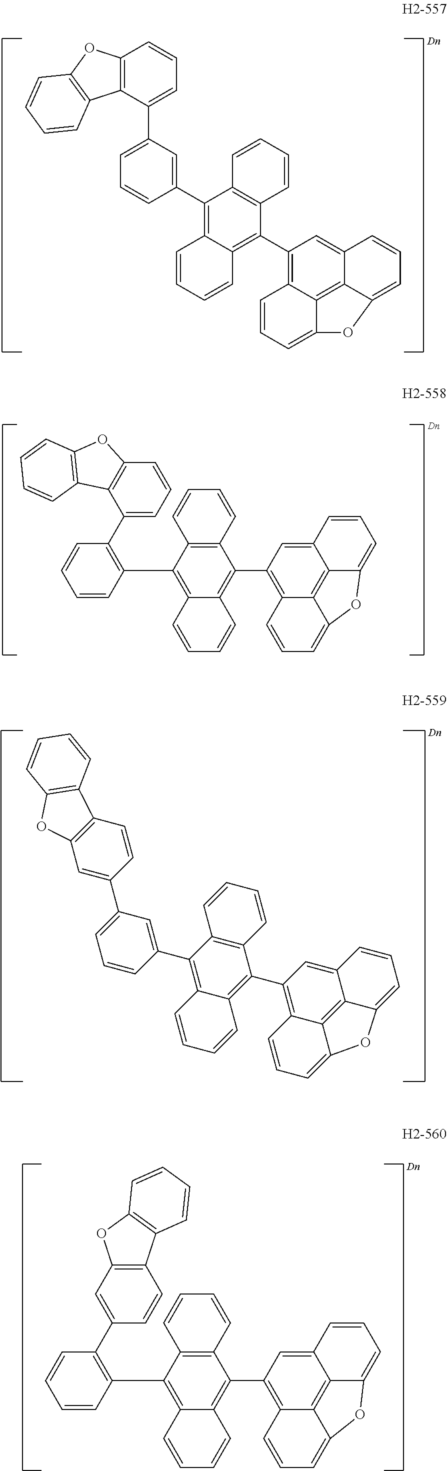 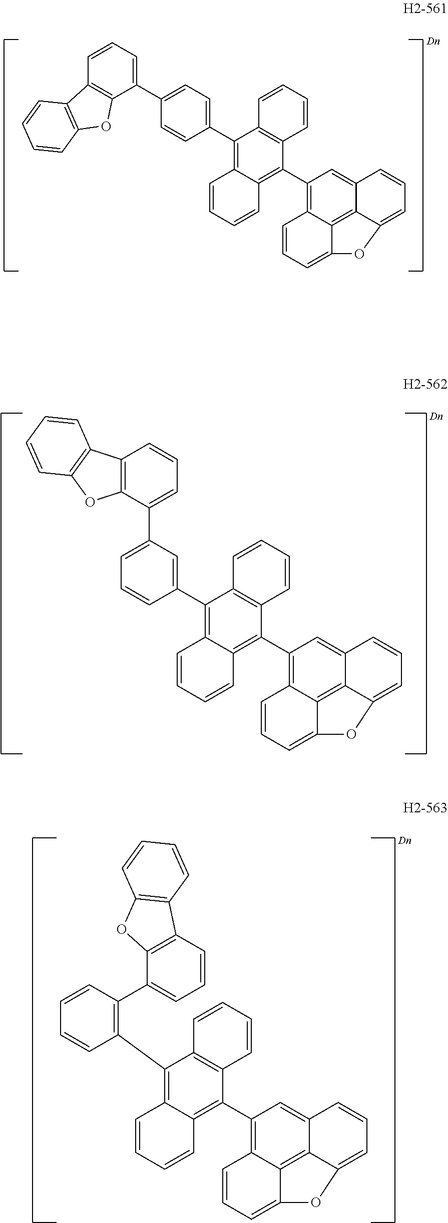 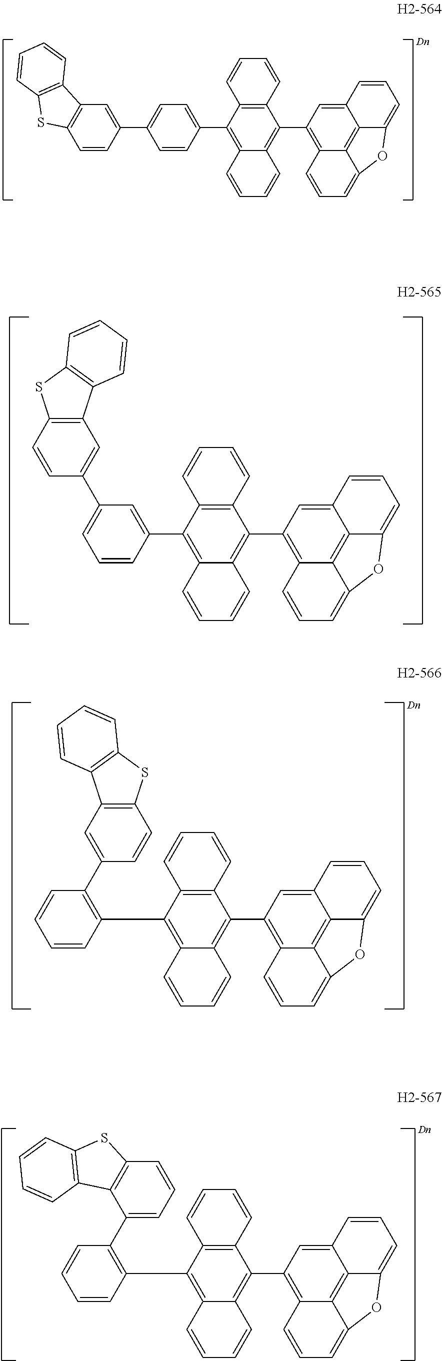 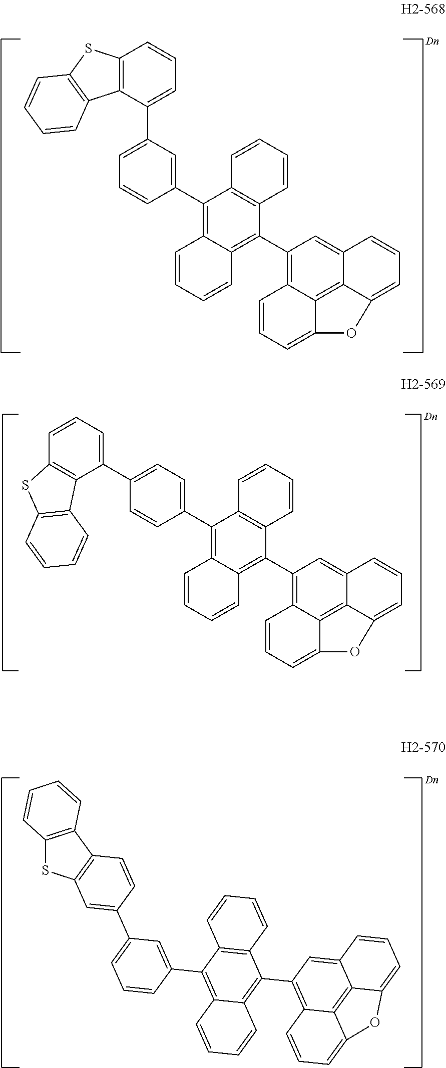 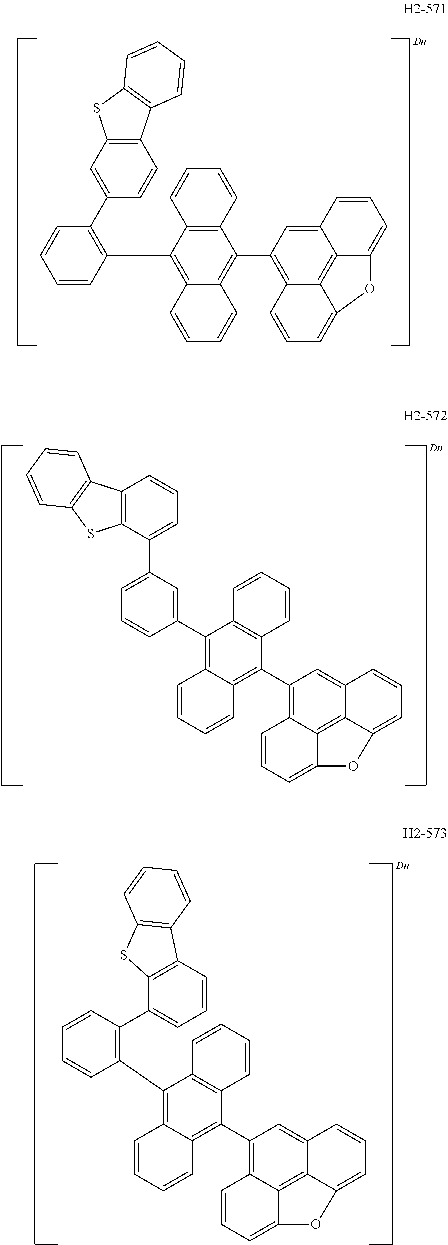 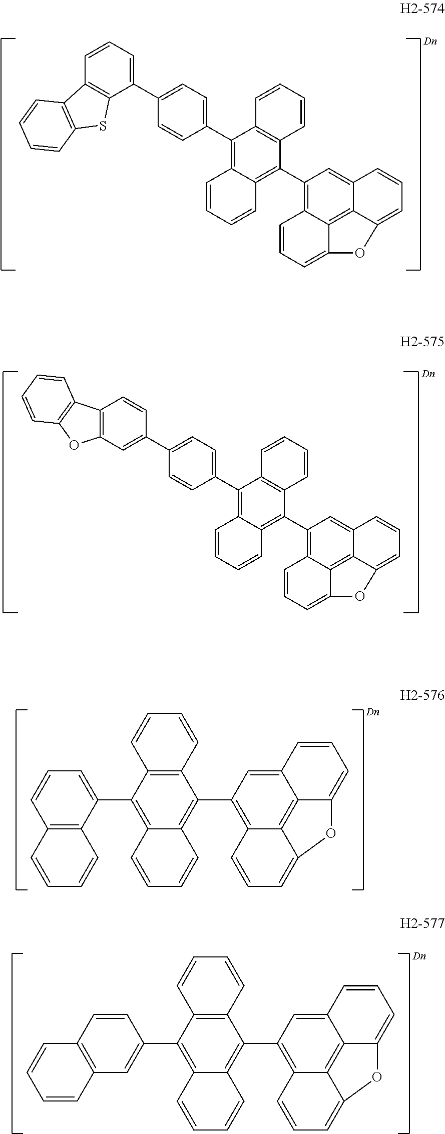 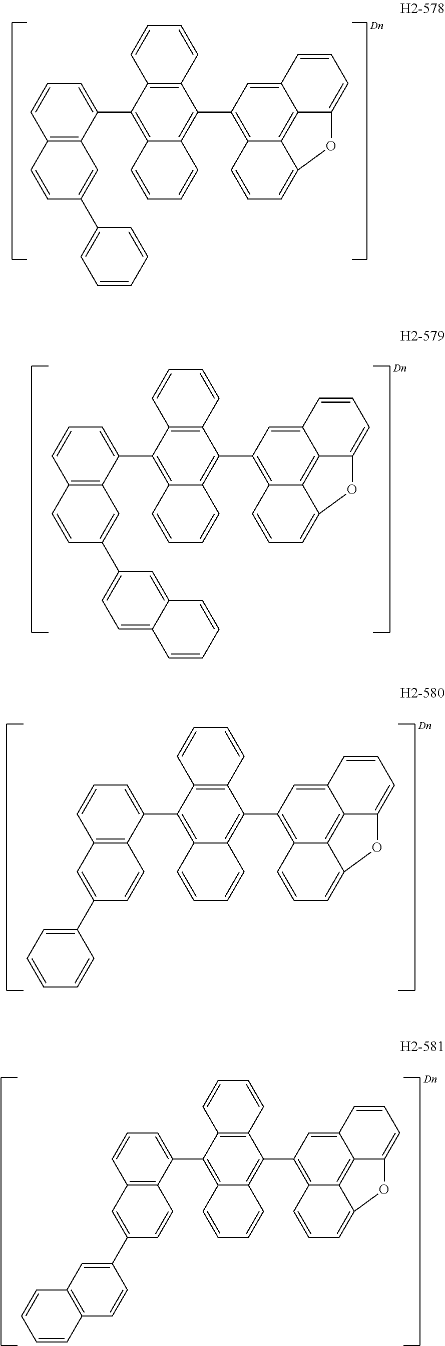 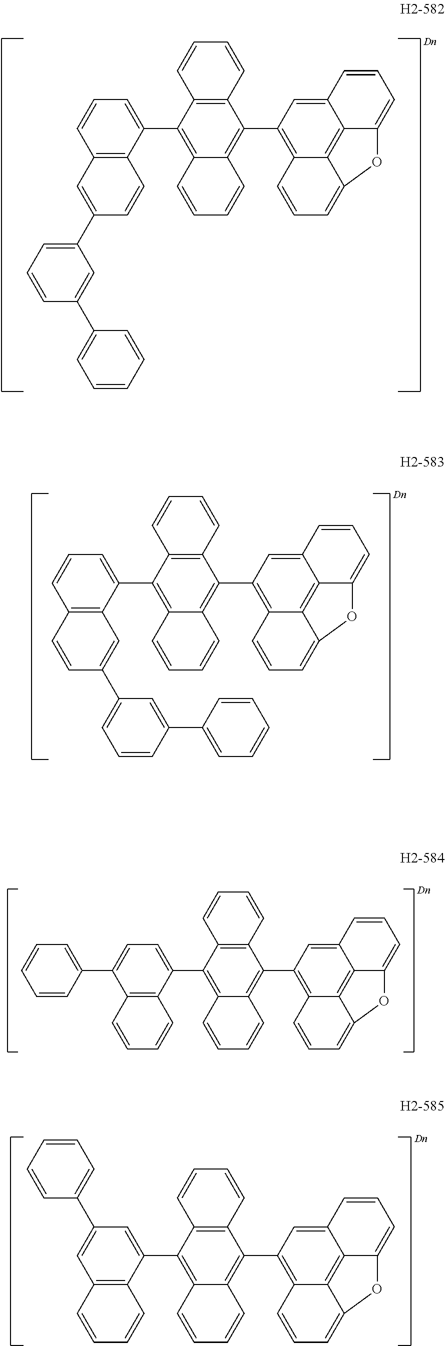 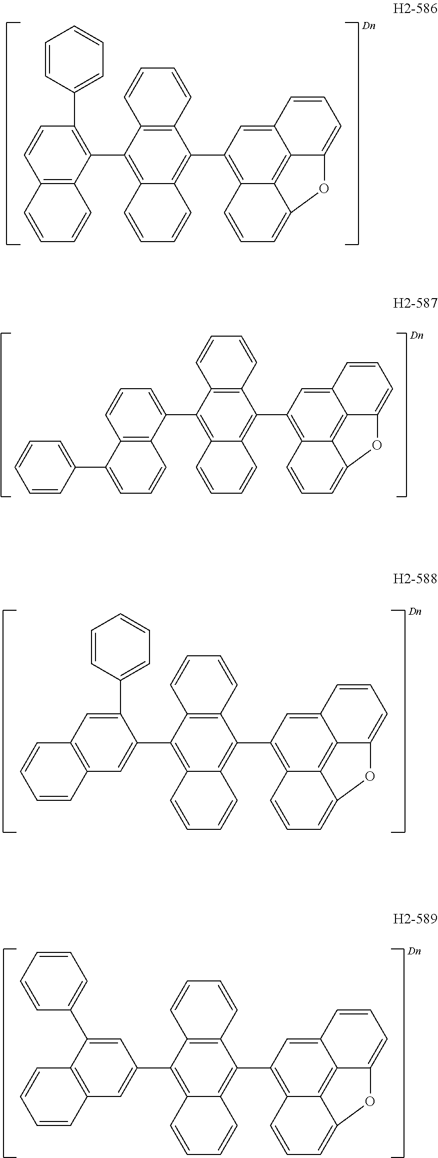 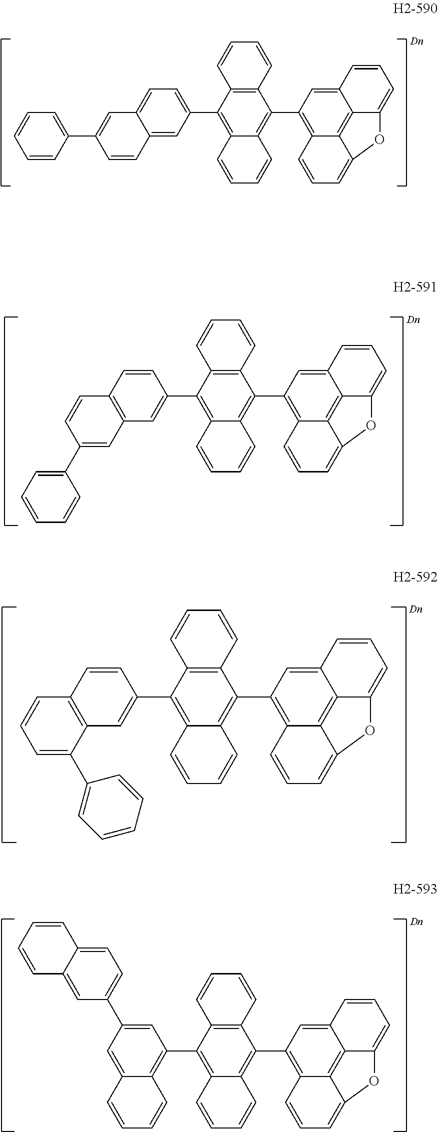 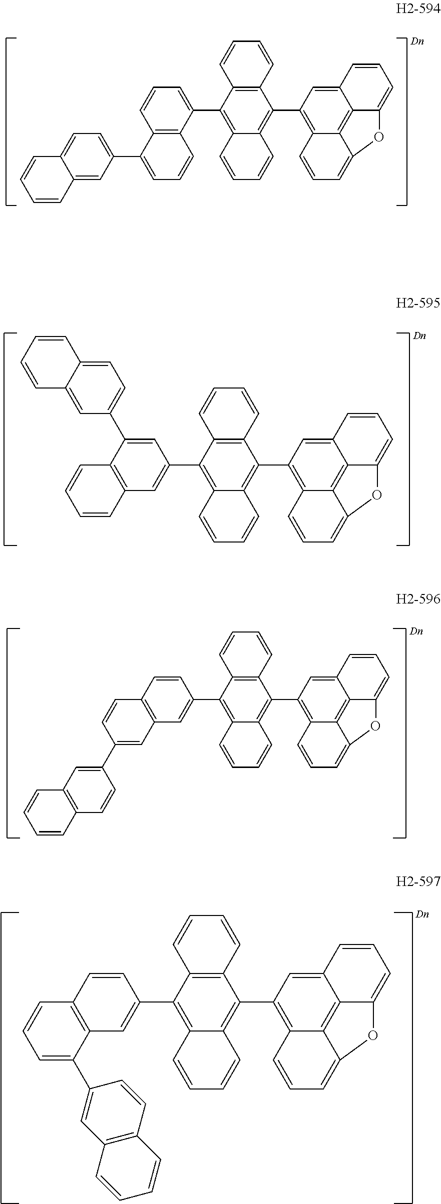 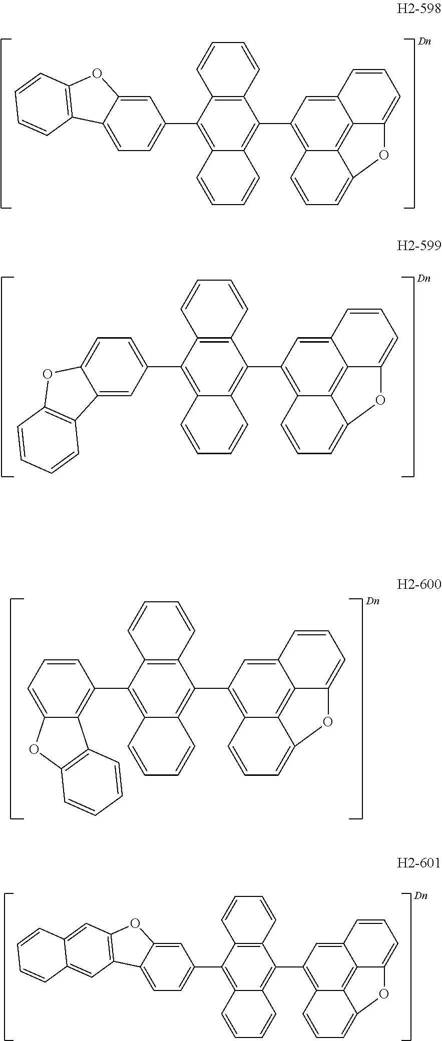 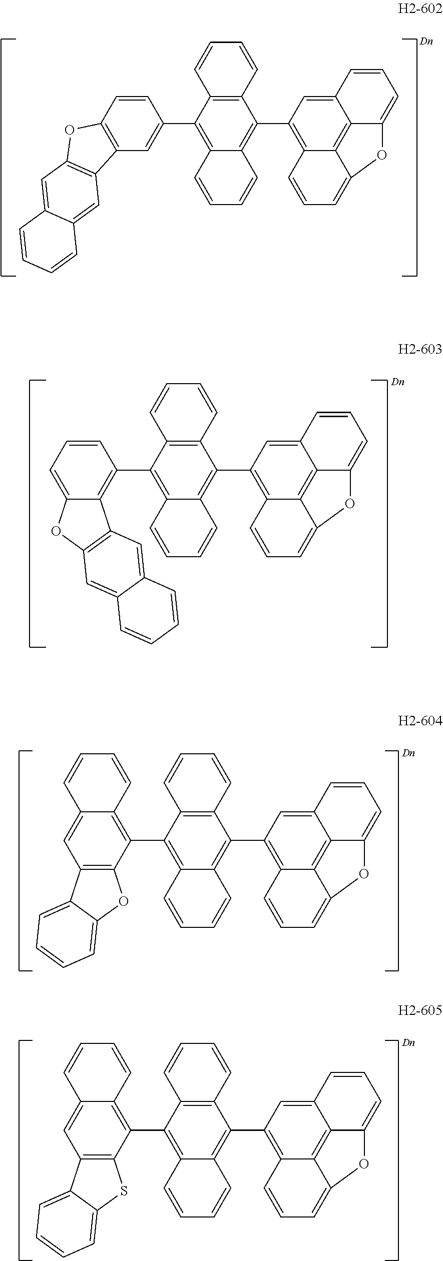 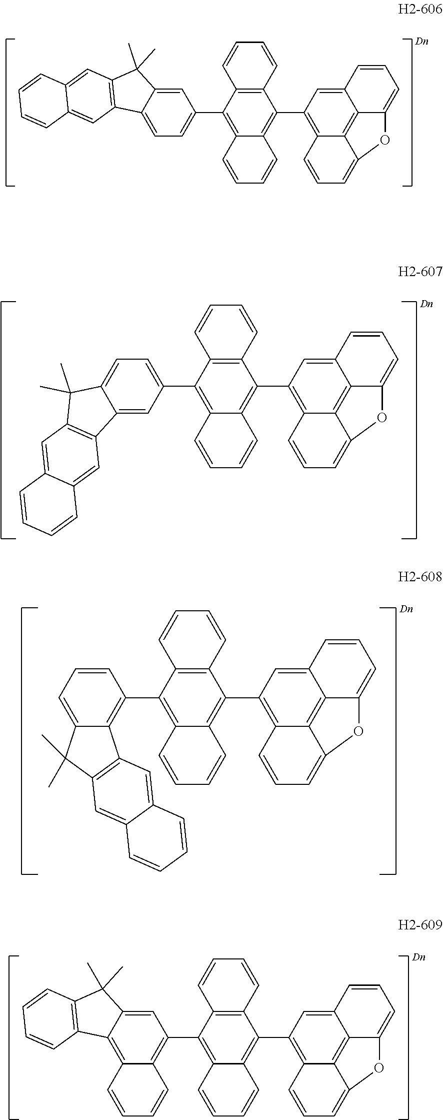 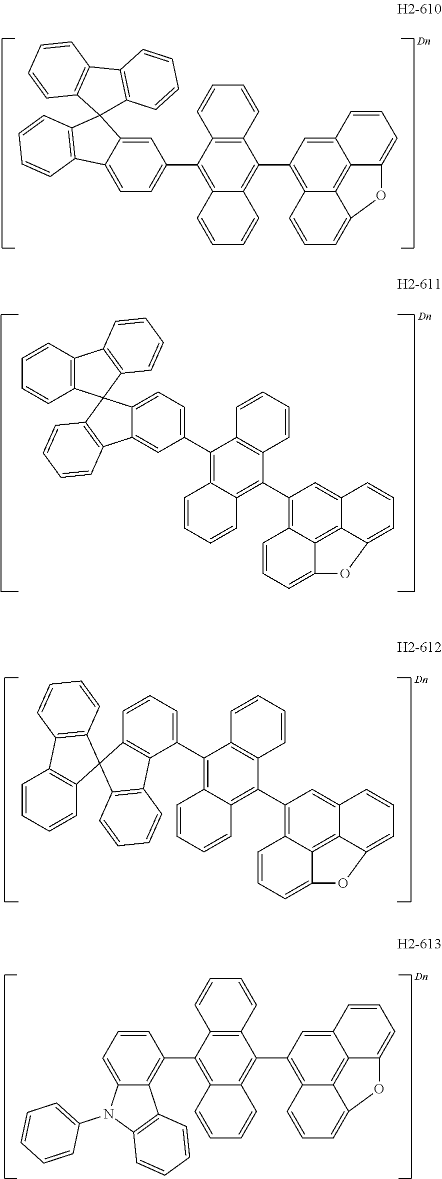 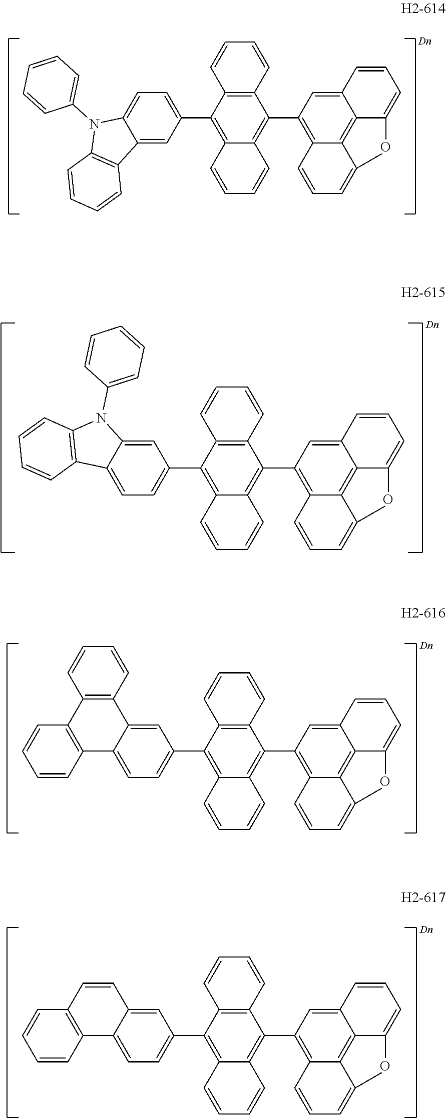 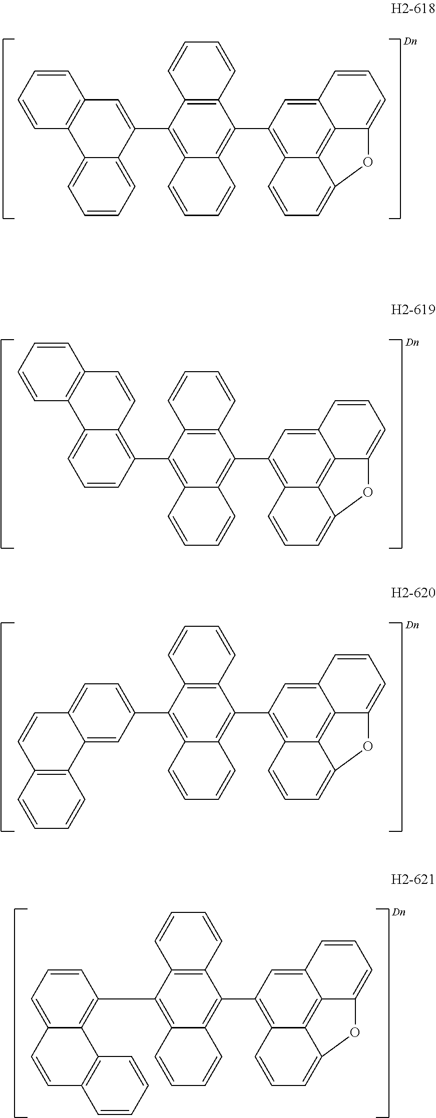 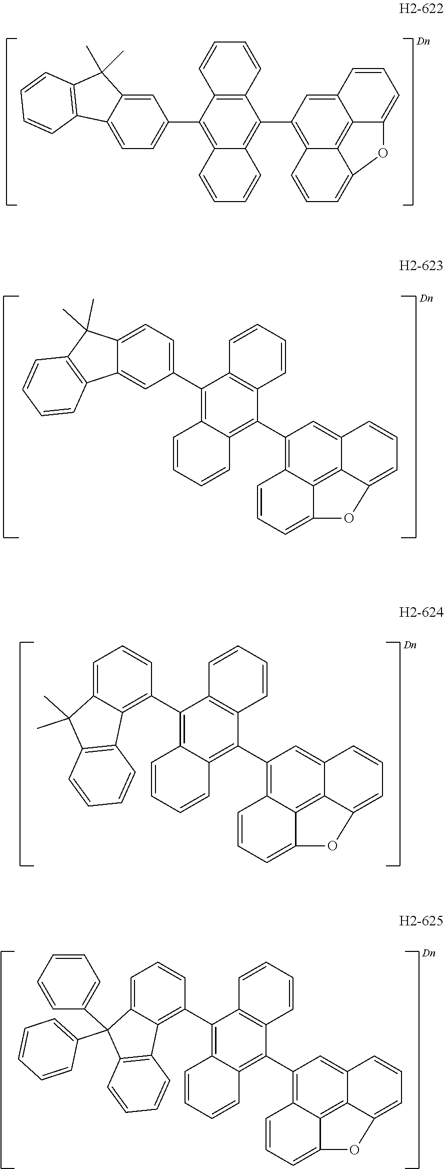 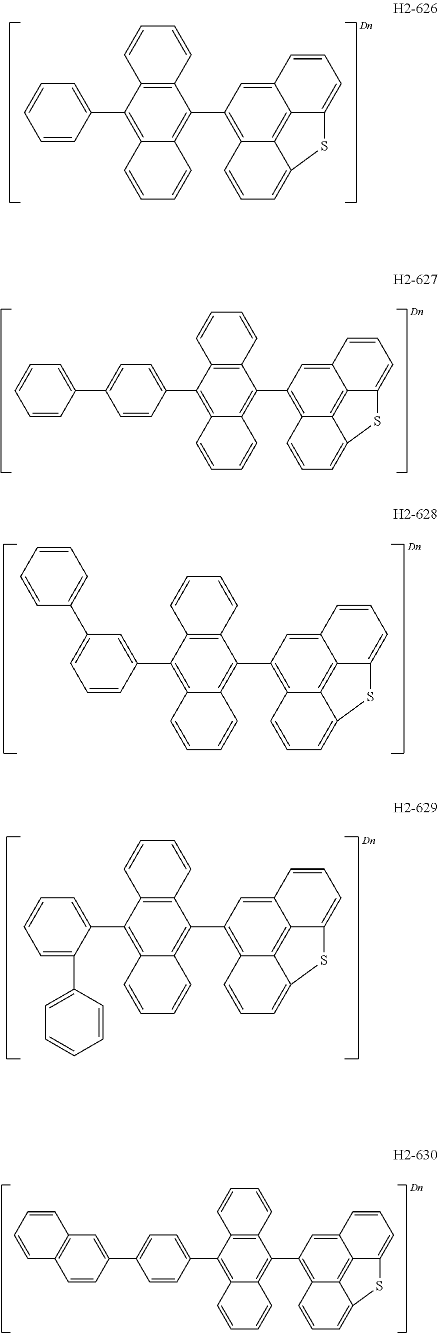 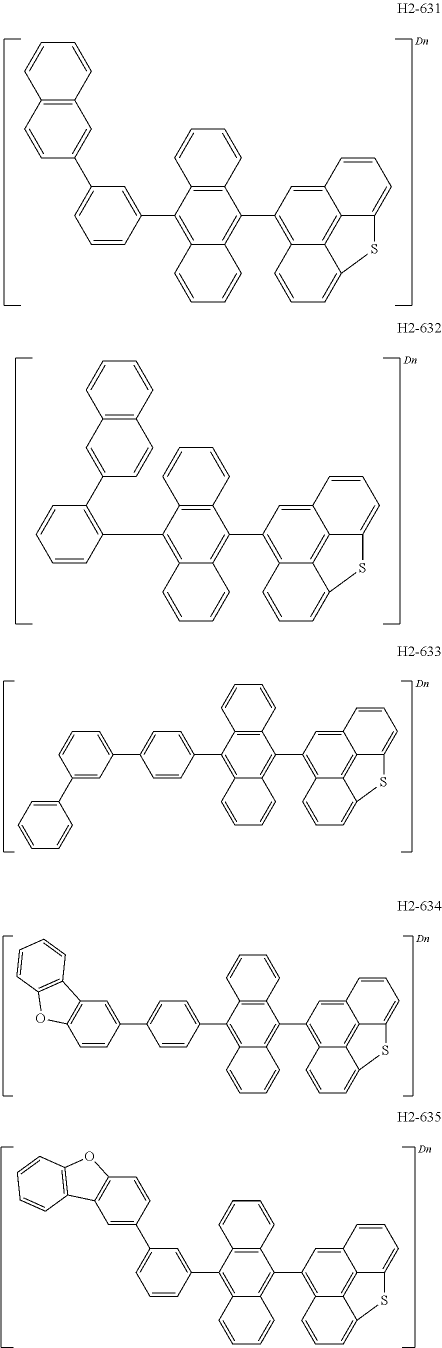 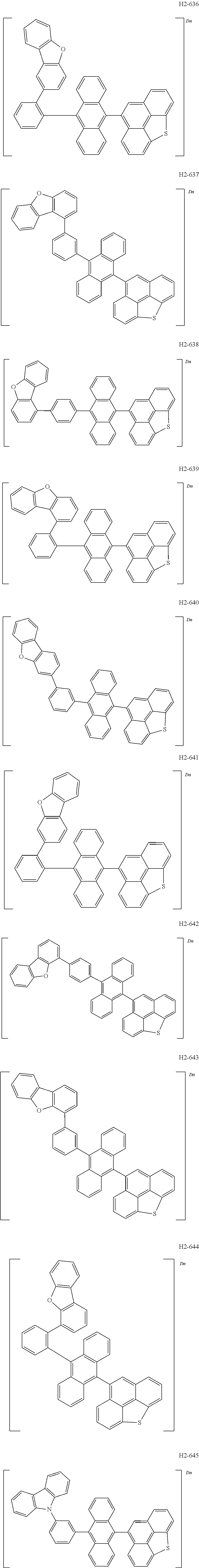 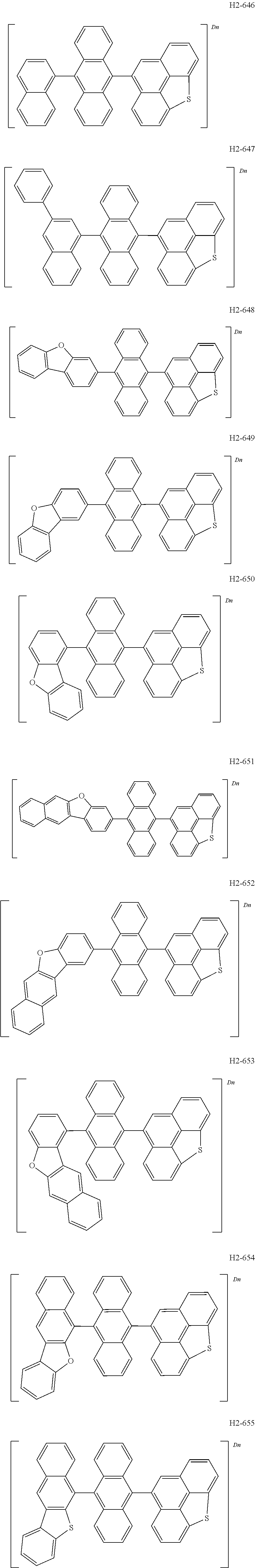 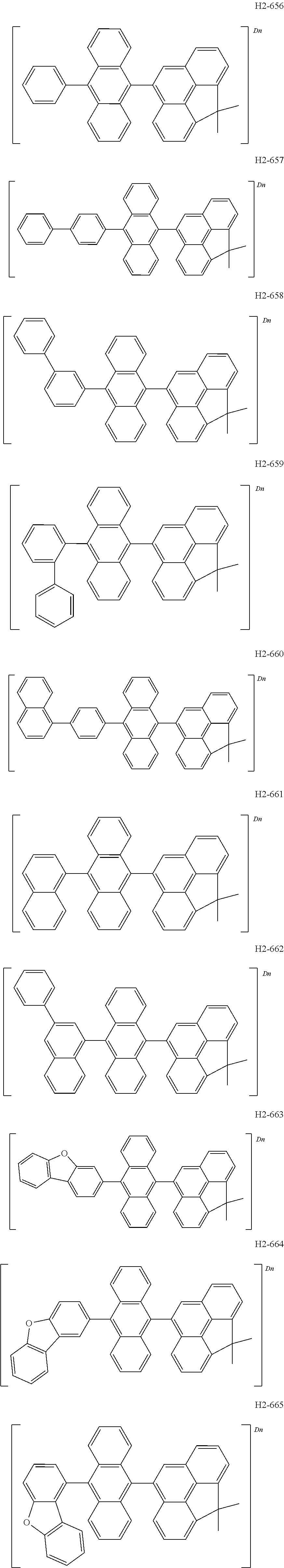 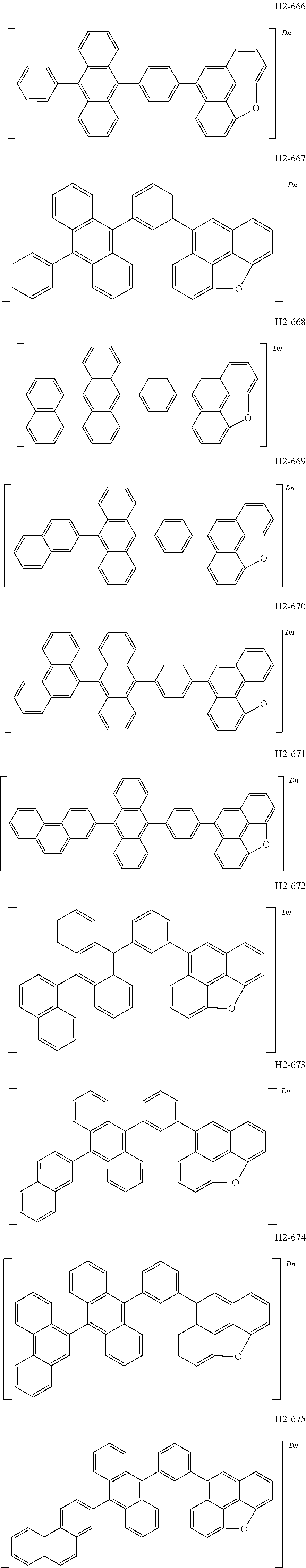 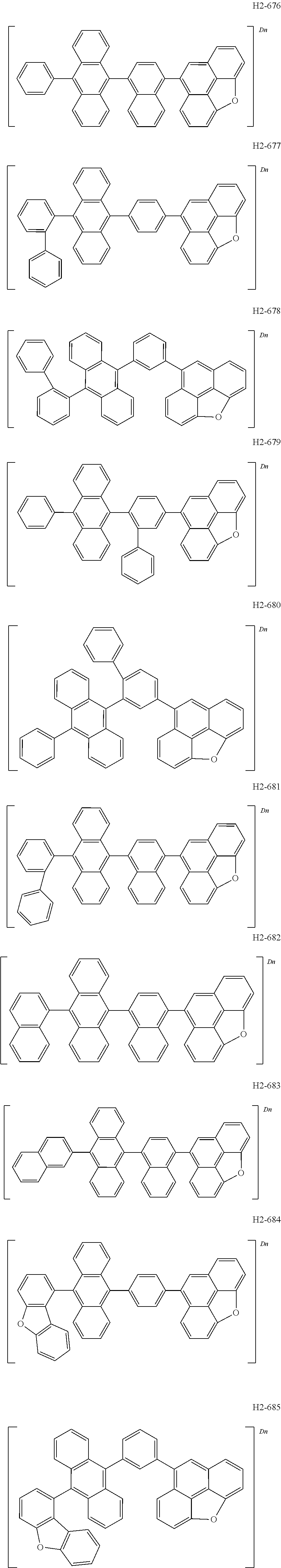 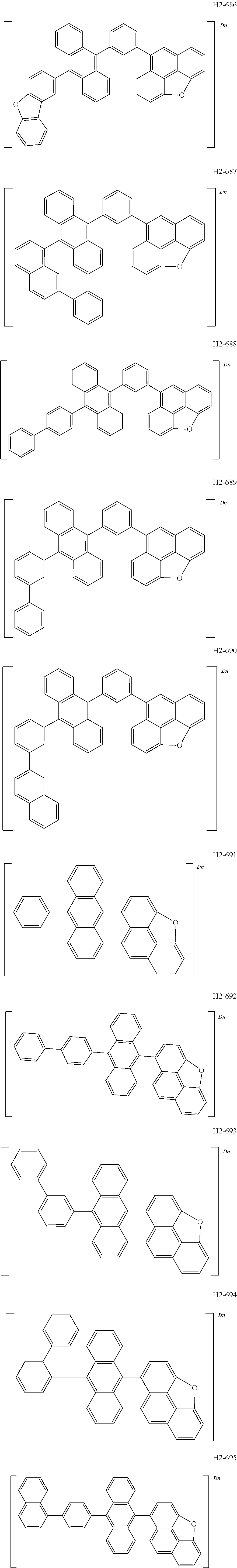 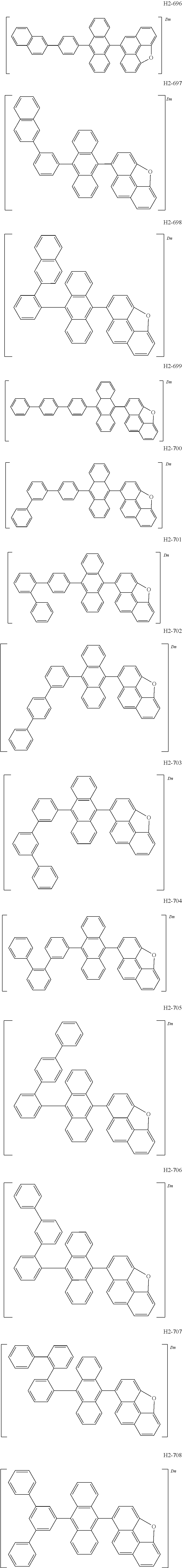 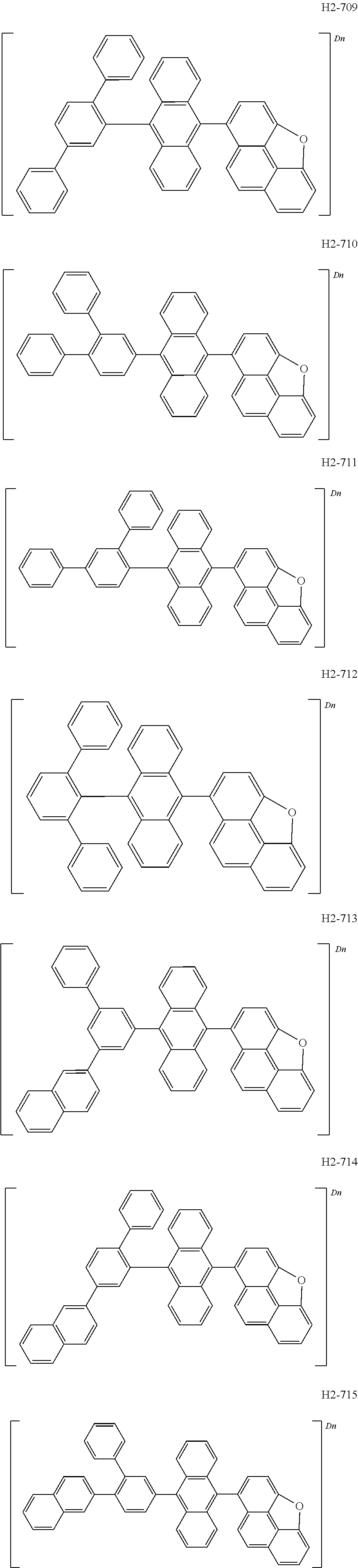 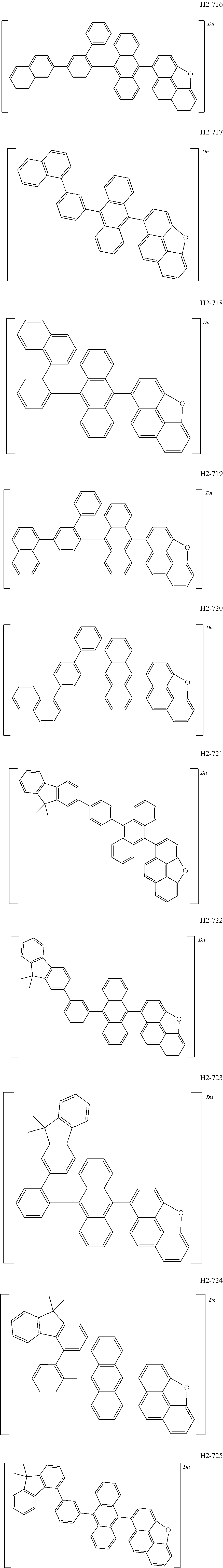 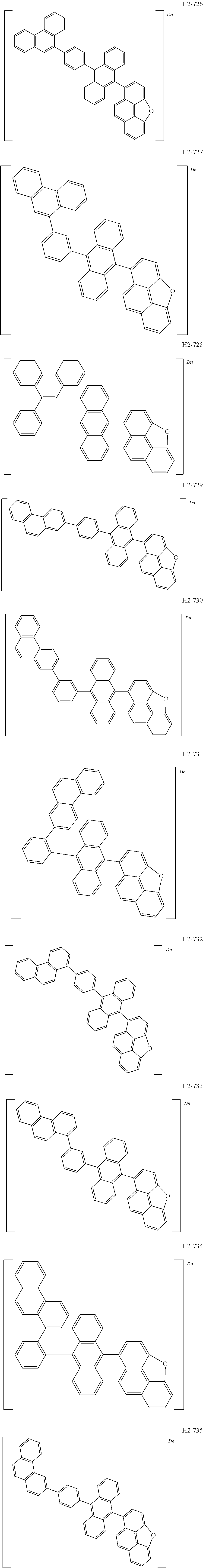 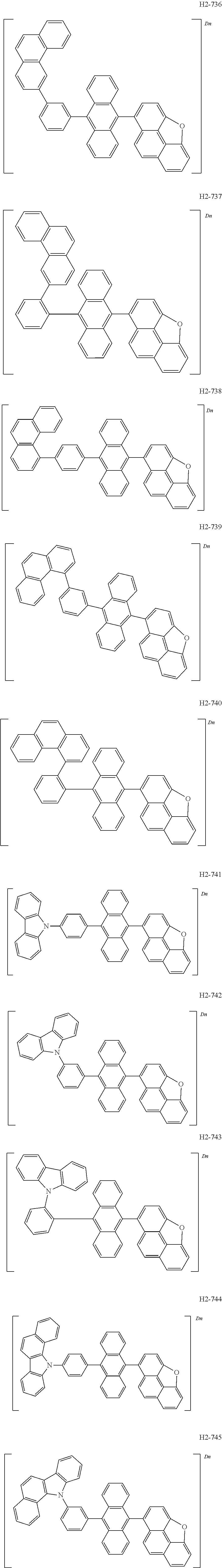 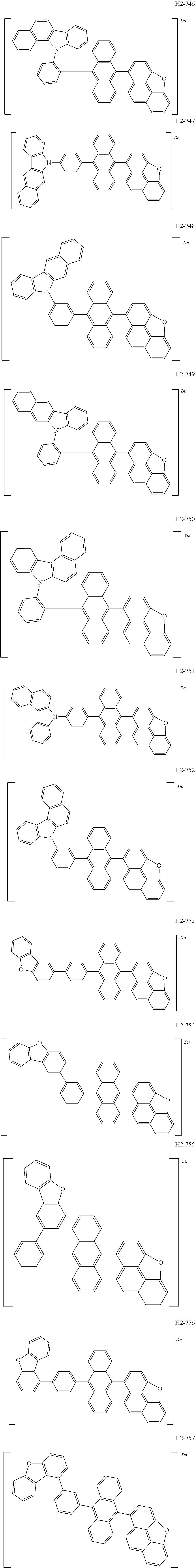 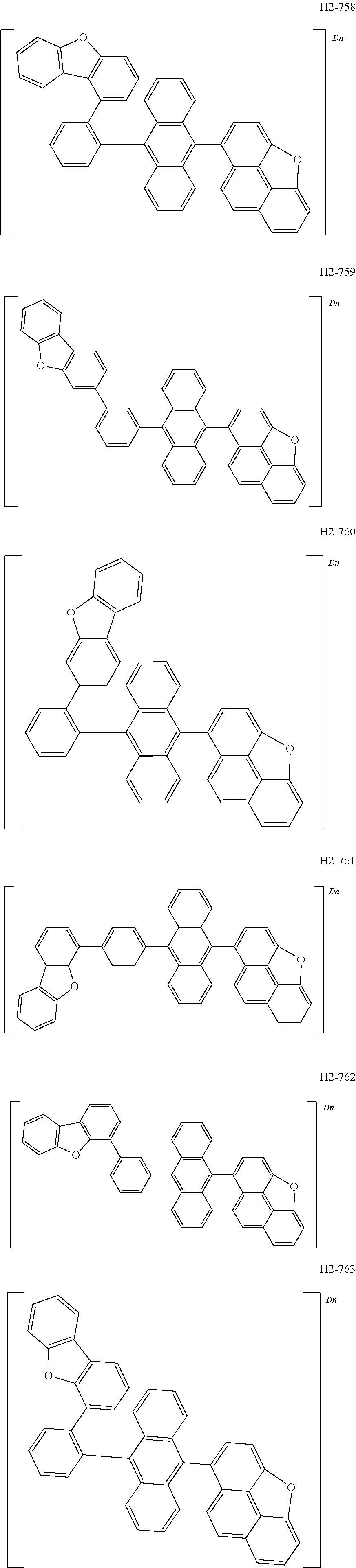 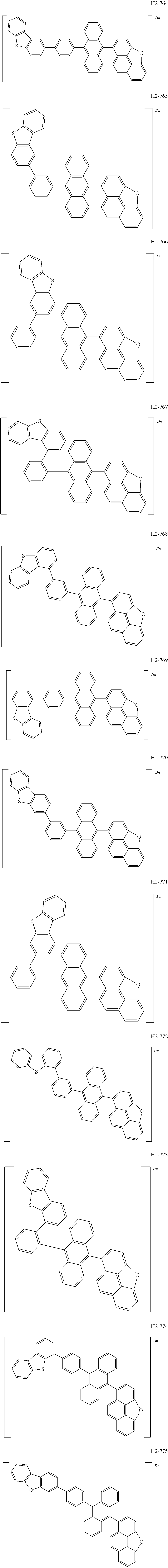 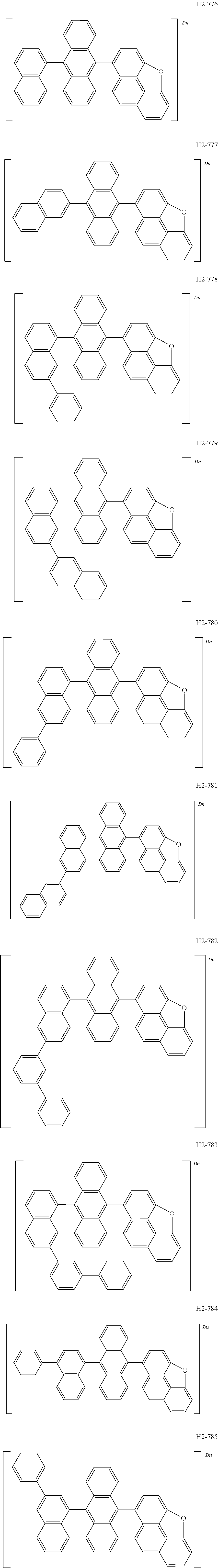 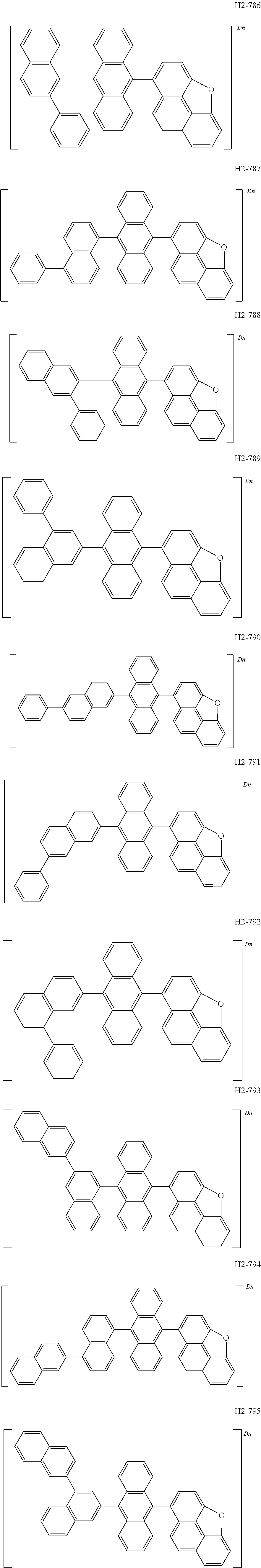 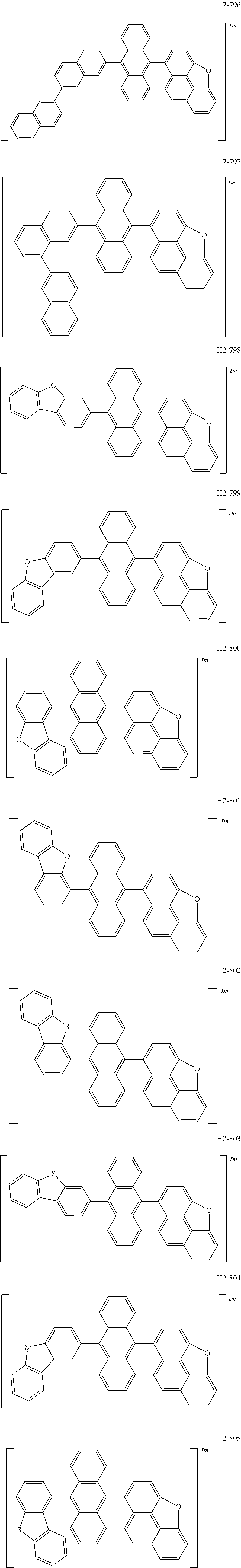 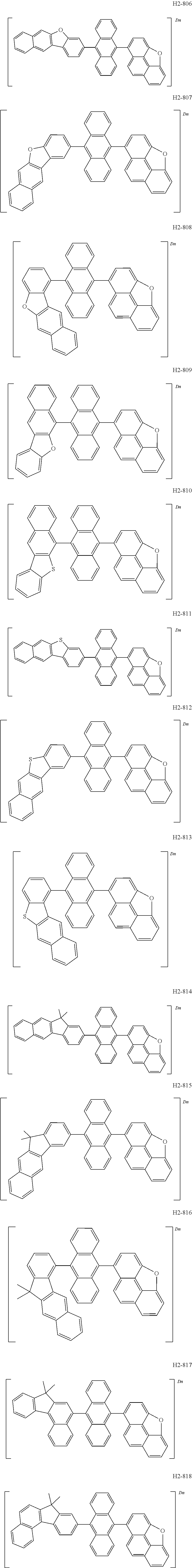 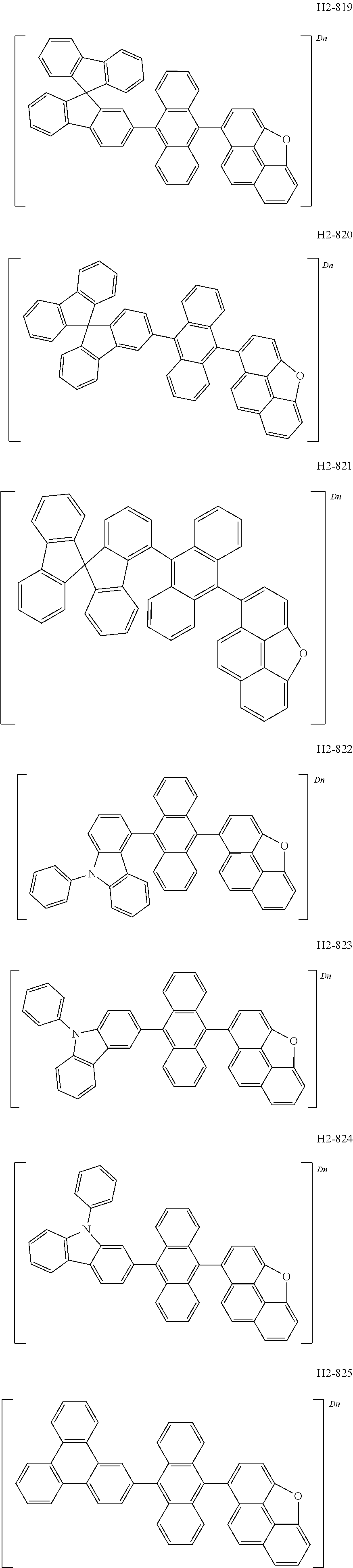 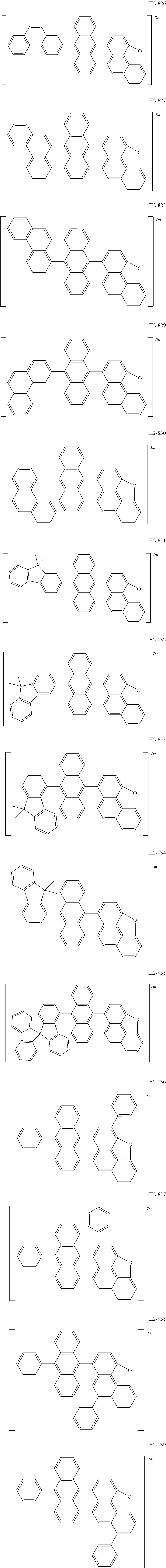 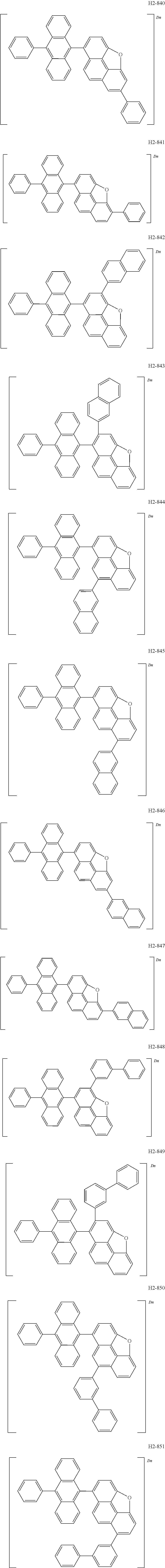 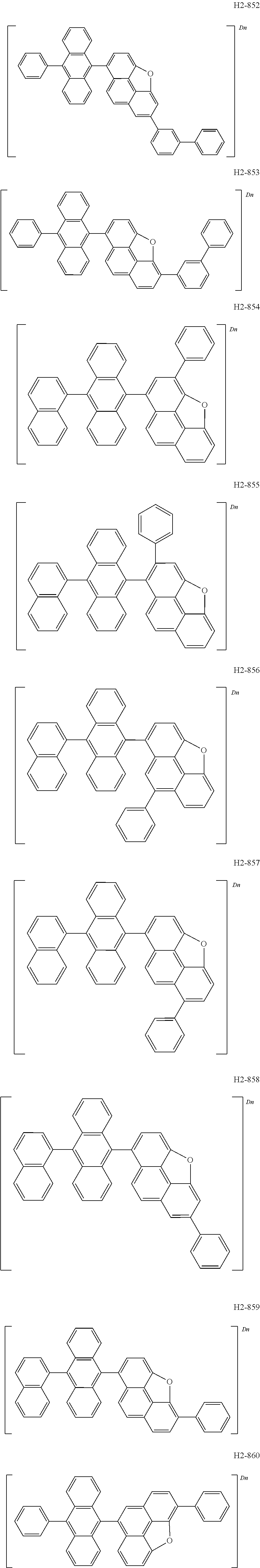 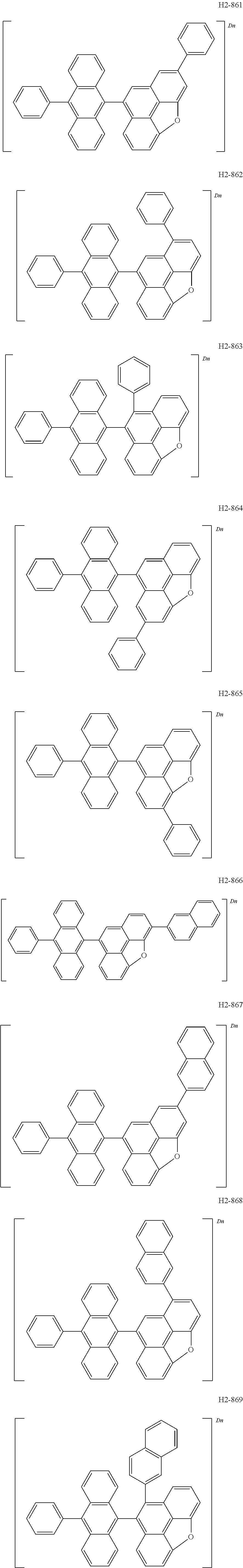 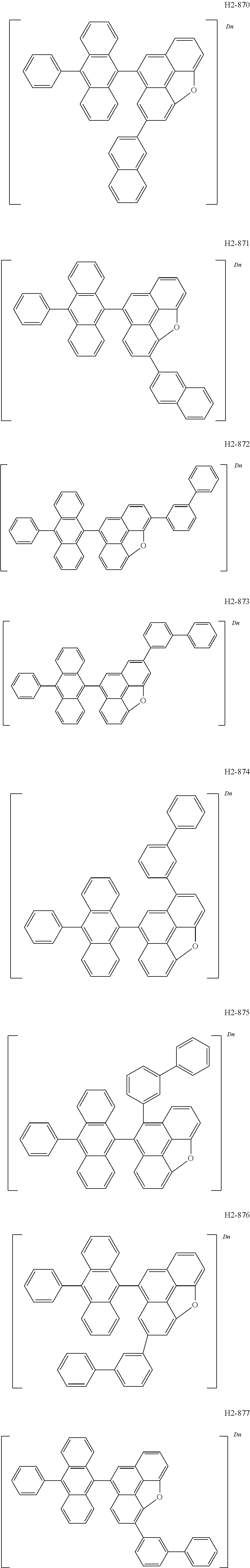 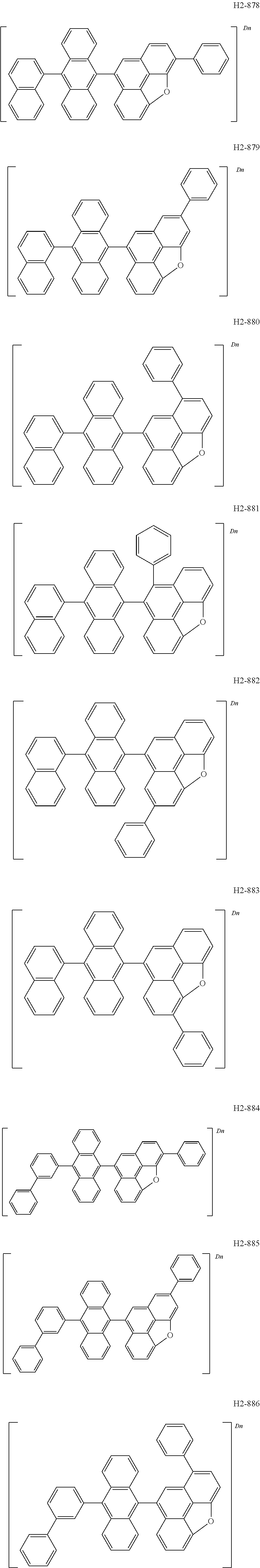 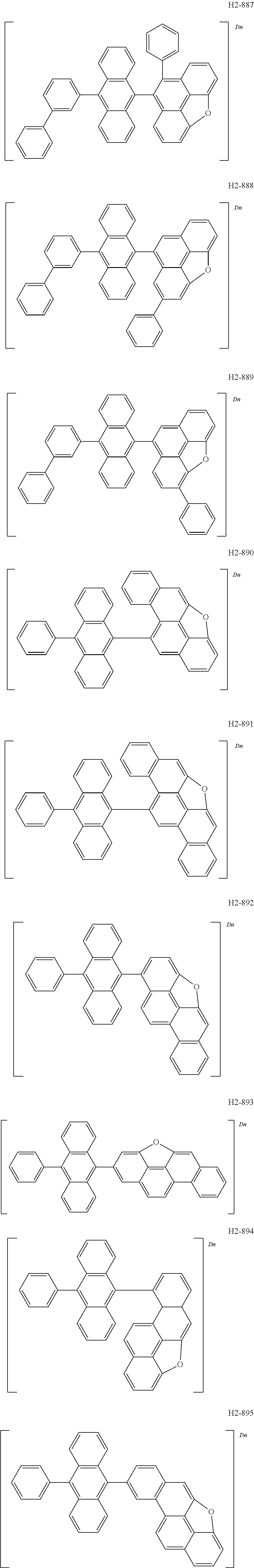 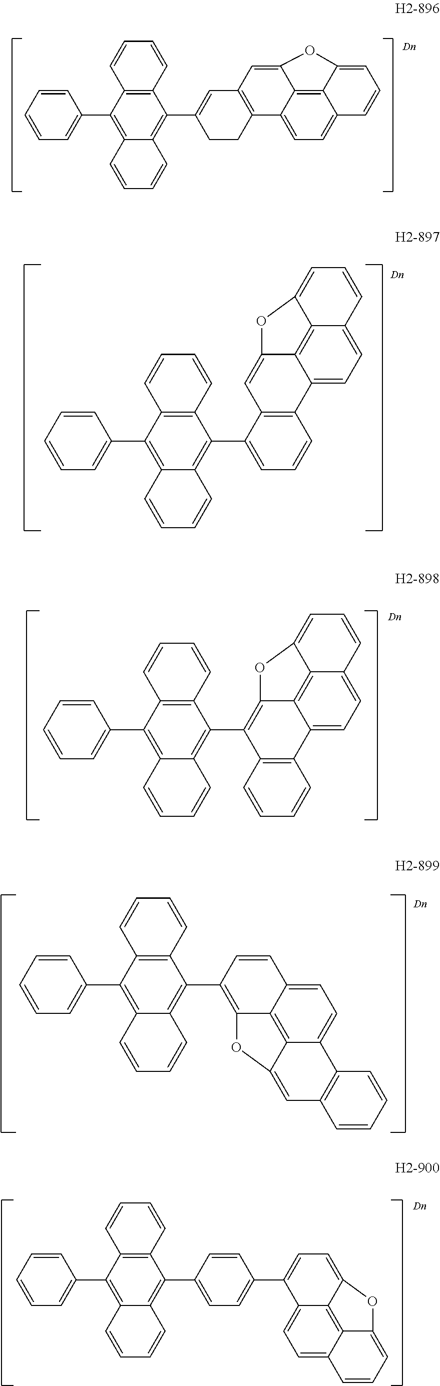 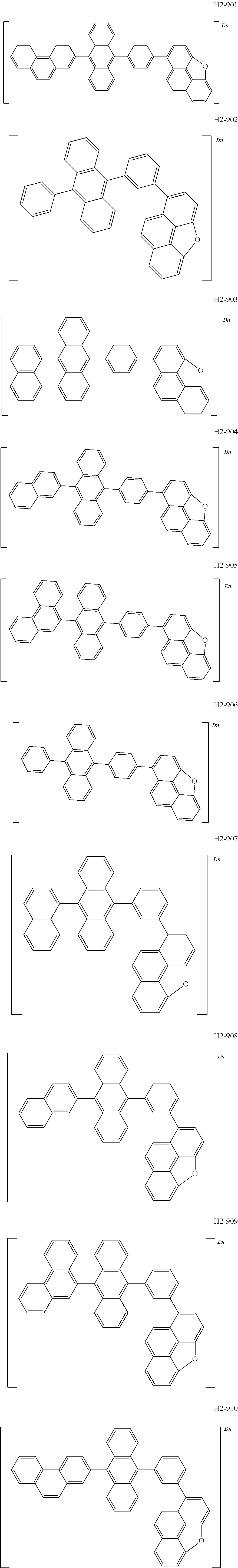 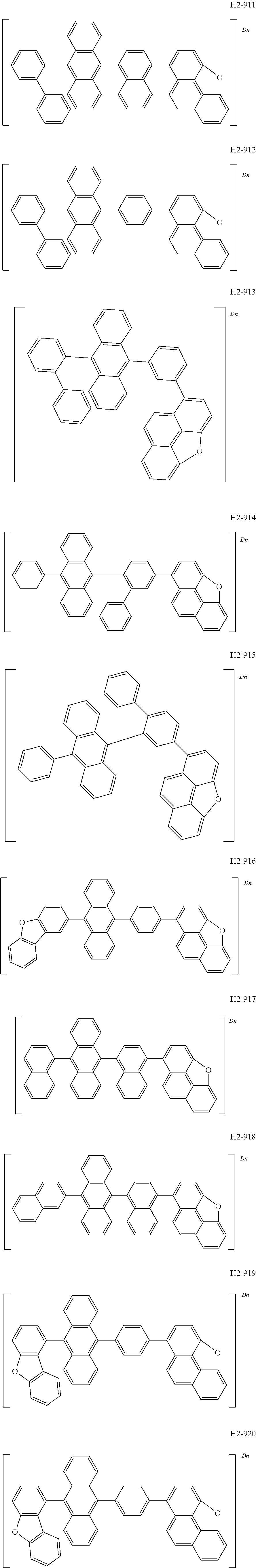 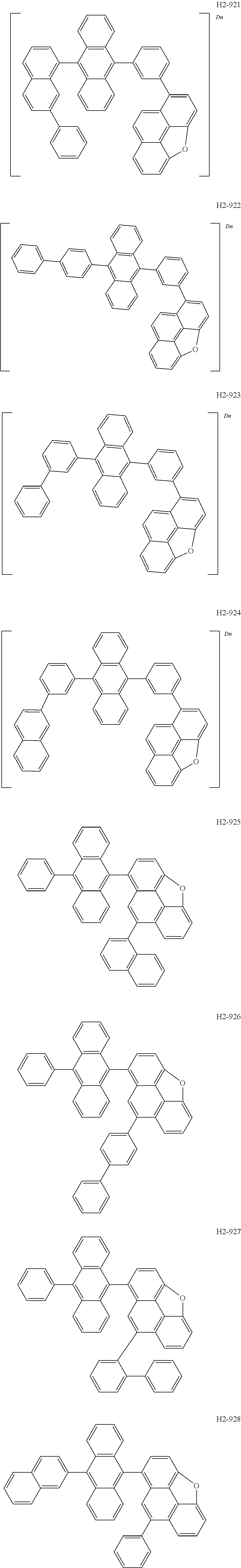 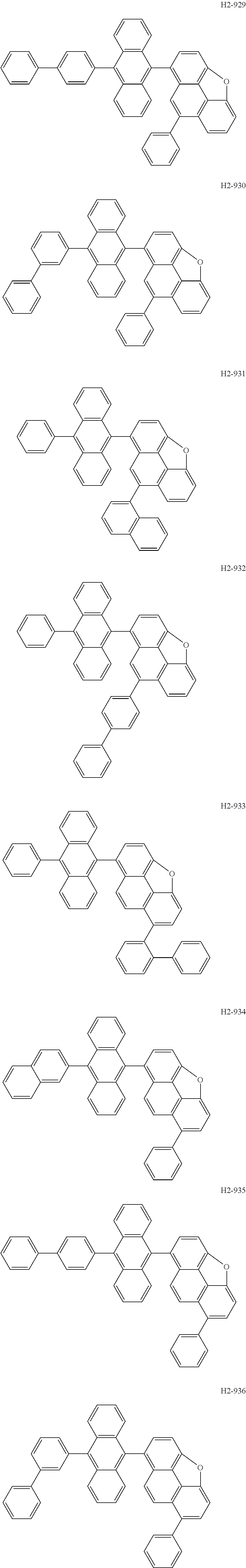 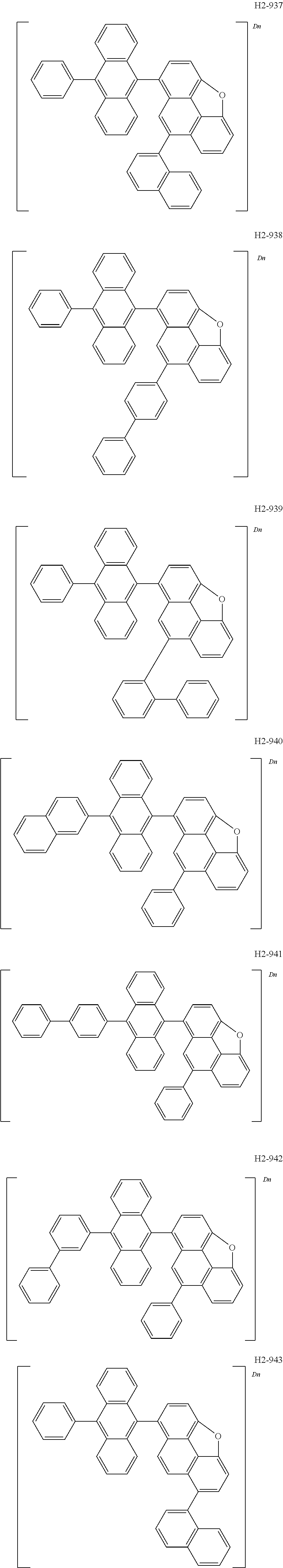 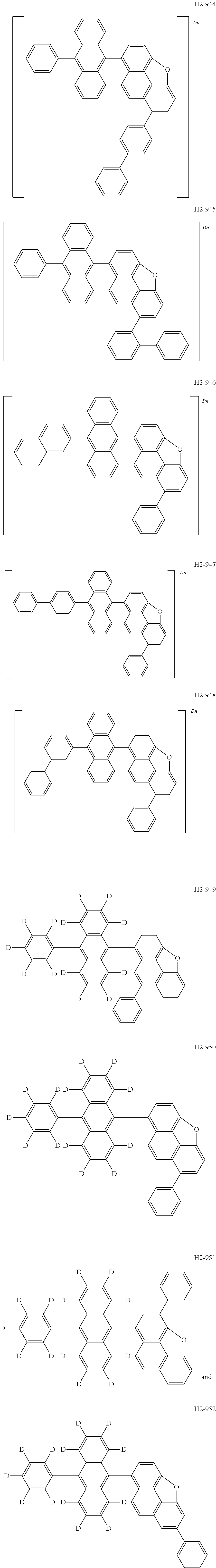
wherein Dₙ means that n hydrogens are substituted with deuterium, and n is an integer from 1 to the maximum number of hydrogens in the compound.

8. An organic electroluminescent device comprising an anode; a cathode; and at least one light-emitting layer between the anode and the cathode, wherein the at least one light-emitting layer comprises the plurality of host materials according to claim 1.

9. An organic electroluminescent compound represented by the following Formula 1-a: 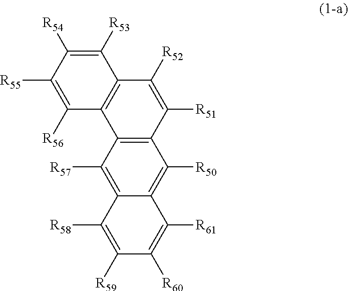
wherein,
R₅₀ to R₆₁ each independently represent hydrogen, deuterium, the following Formula 1-b, or the following Formula 1-c, provided that at least one of R₅₀ to R₆₁ represents the following Formula 1-b or Formula 1-c: 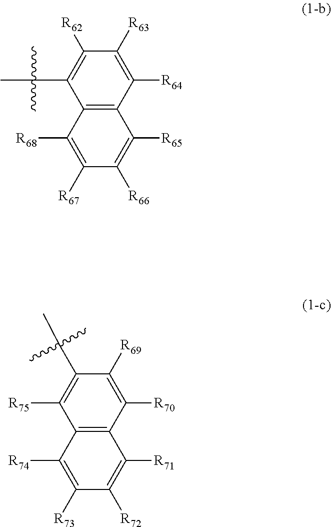
wherein,
R₆₂ to R₆₅ and R₆₈ each independently represent hydrogen, deuterium, a phenyl unsubstituted or substituted with deuterium, a biphenyl unsubstituted or substituted with deuterium, or a naphthyl unsubstituted or substituted with deuterium, and at least one of R₆₂ to R₆₅ and R₆₈ represents a phenyl unsubstituted or substituted with deuterium, a biphenyl unsubstituted or substituted with deuterium, or a naphthyl unsubstituted or substituted with deuterium;
R₆₆ and R₆₇ each independently represent hydrogen or deuterium; and
R₆₉ to R₇₅ each independently represent hydrogen, deuterium, a phenyl unsubstituted or substituted with deuterium, a biphenyl unsubstituted or substituted with deuterium, or a naphthyl unsubstituted or substituted with deuterium, and at least one of R₆₉ to R₇₅ represents a phenyl unsubstituted or substituted with deuterium, a biphenyl unsubstituted or substituted with deuterium, or a naphthyl unsubstituted or substituted with deuterium.

10. The organic electroluminescent compound according to claim 9, wherein the compound represented by Formula 1-a is selected from the following compounds: 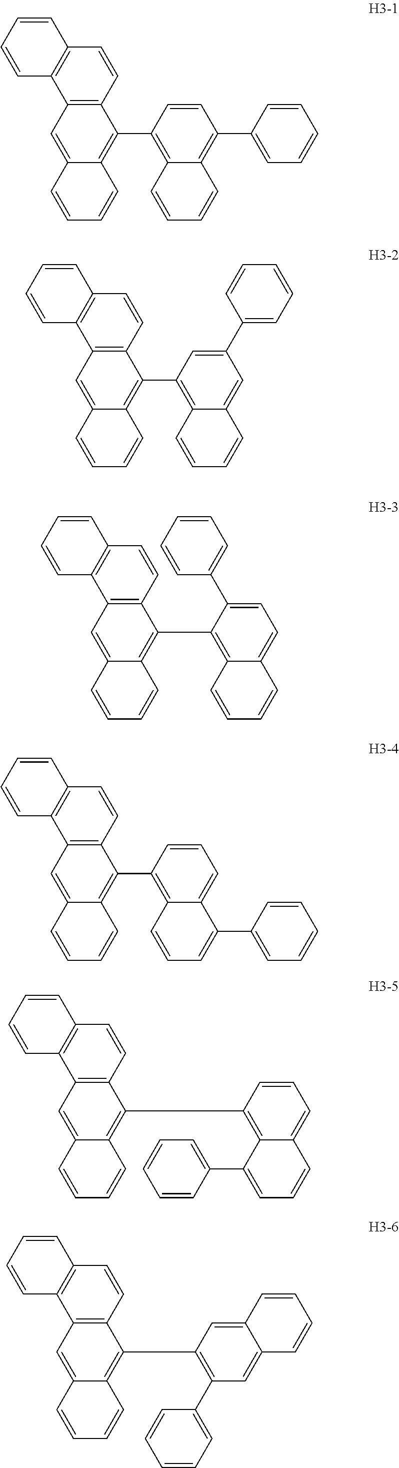 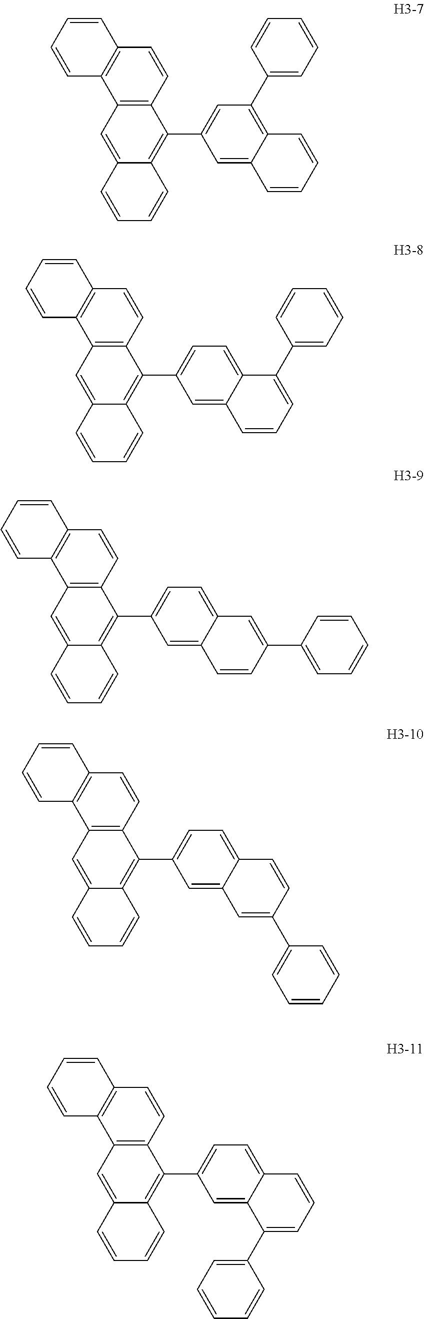 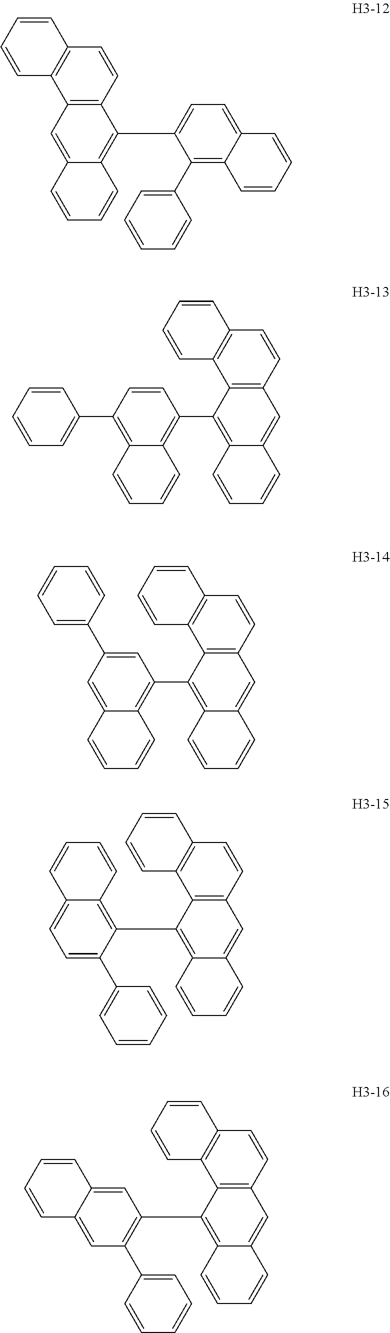 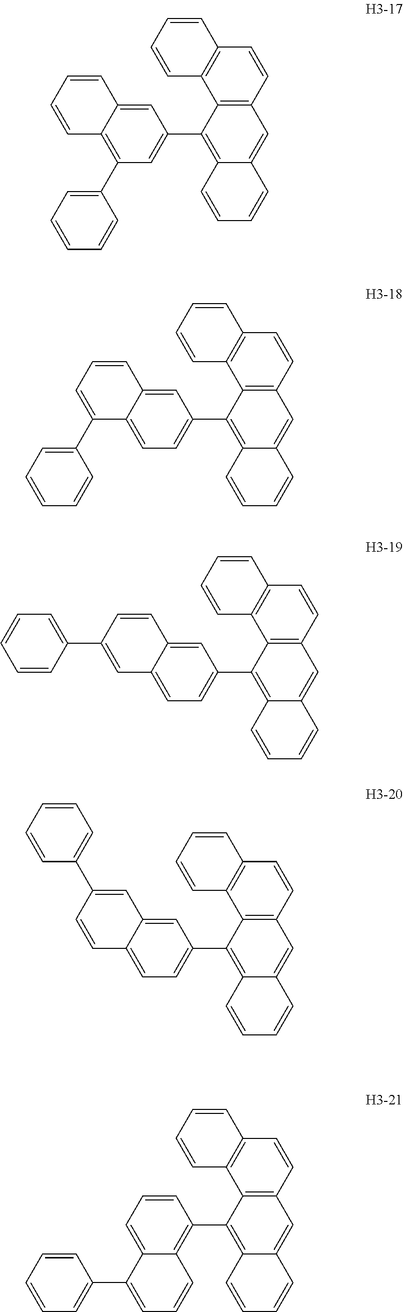 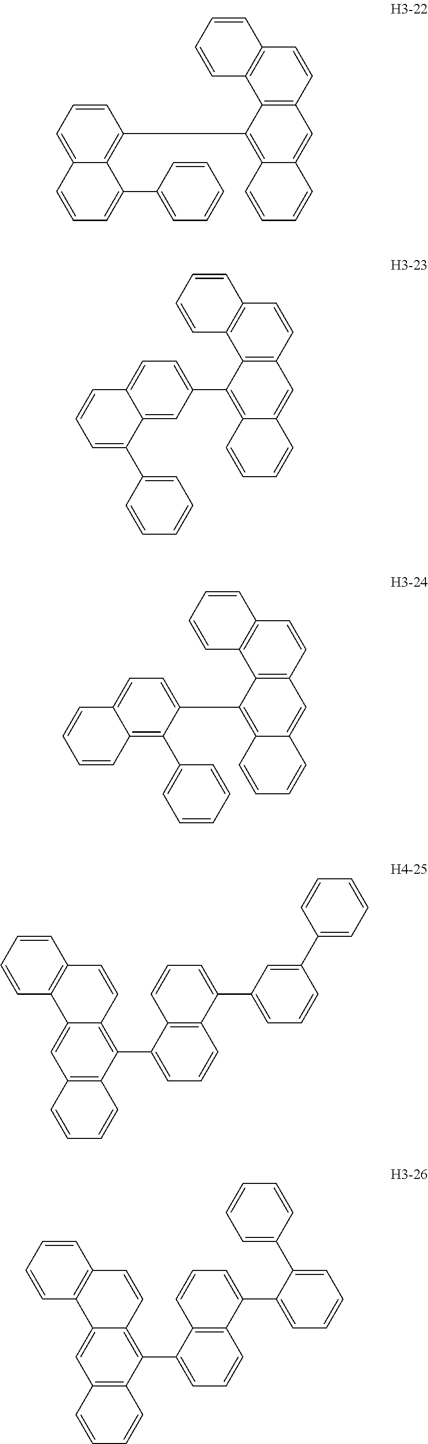 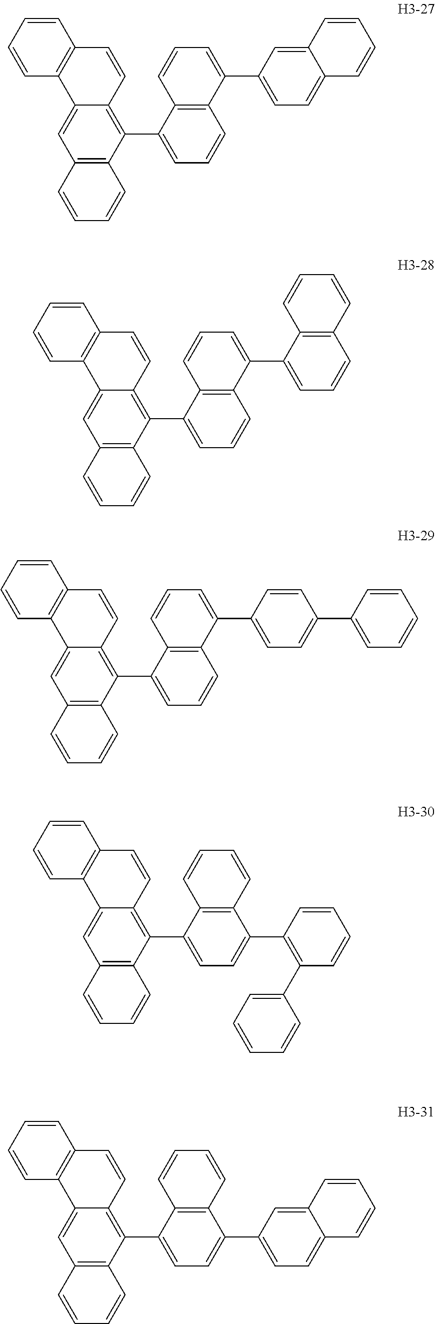 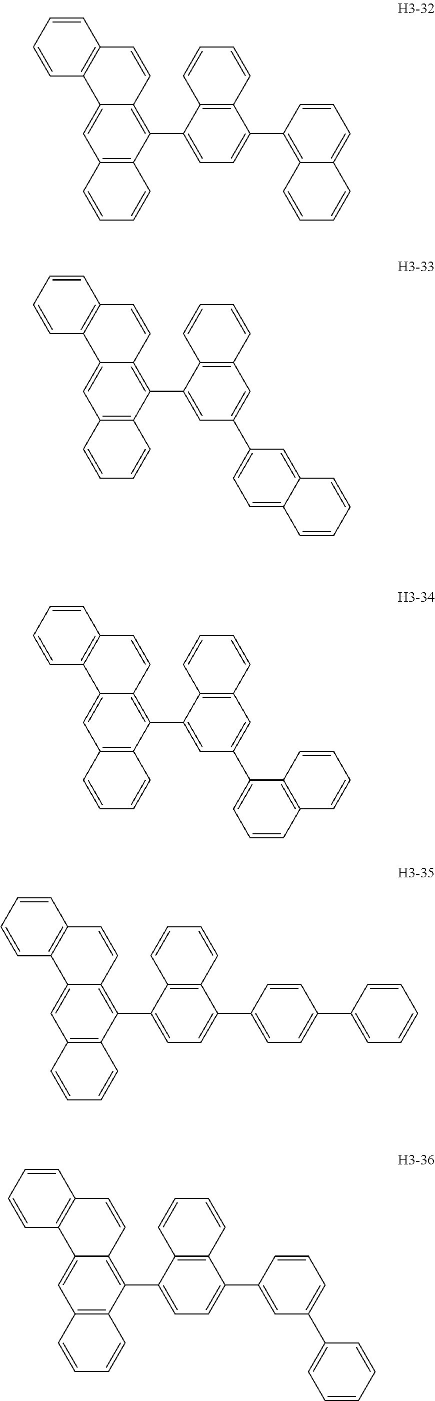 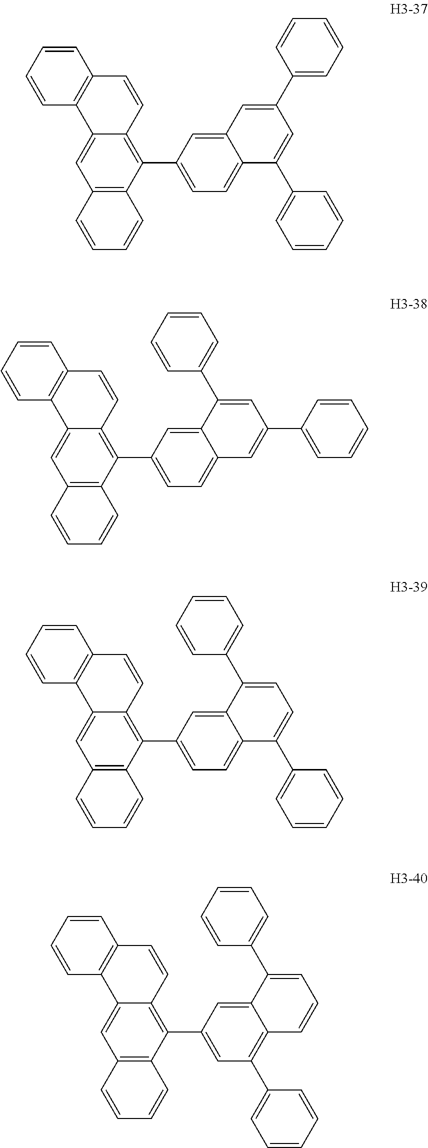 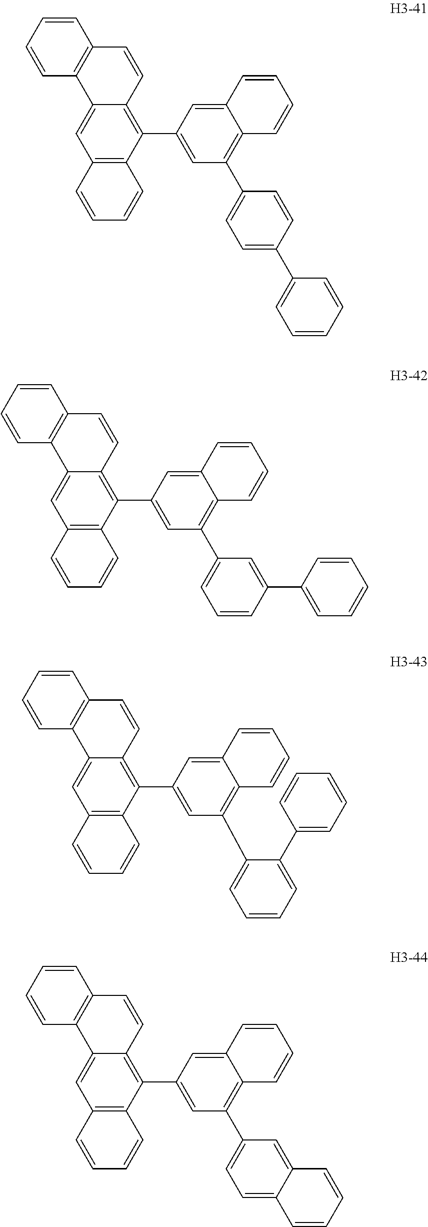 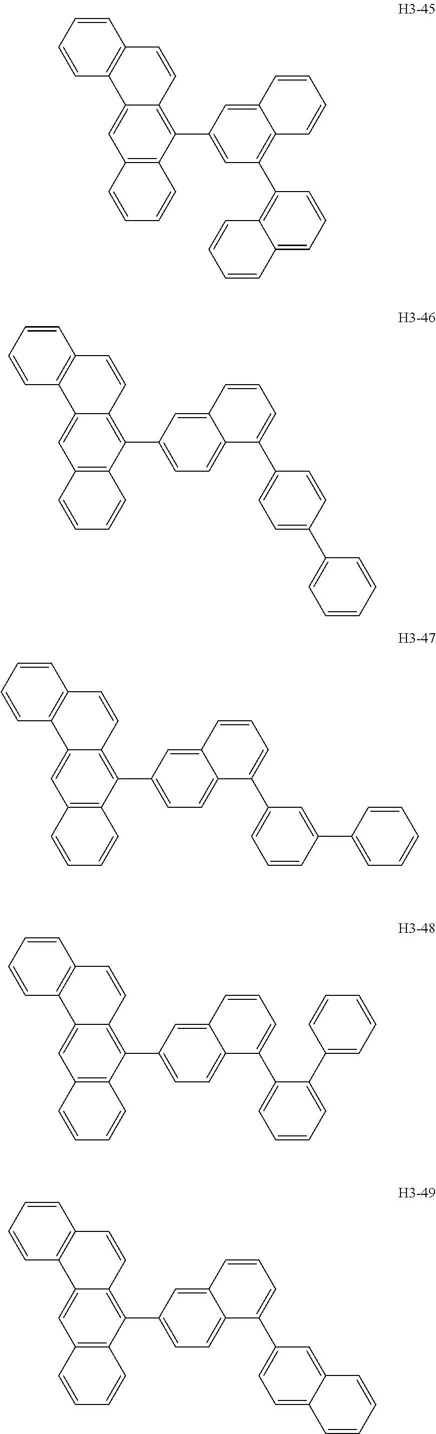 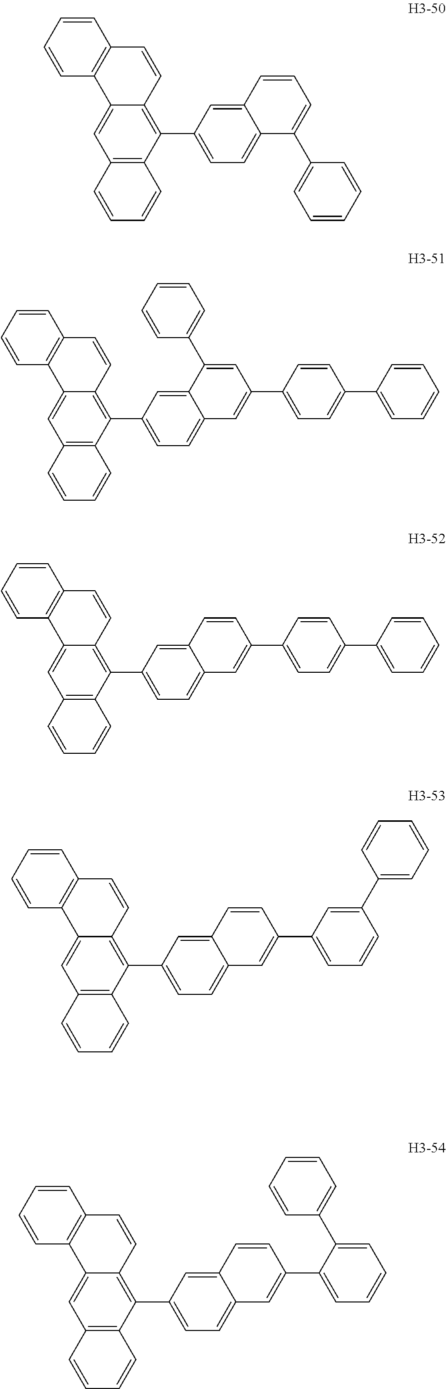 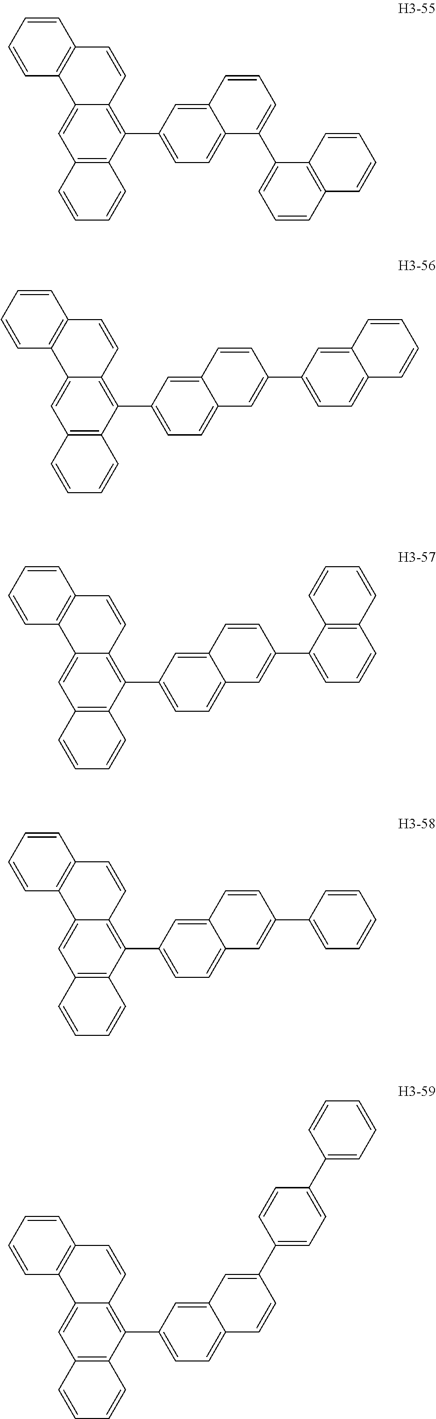 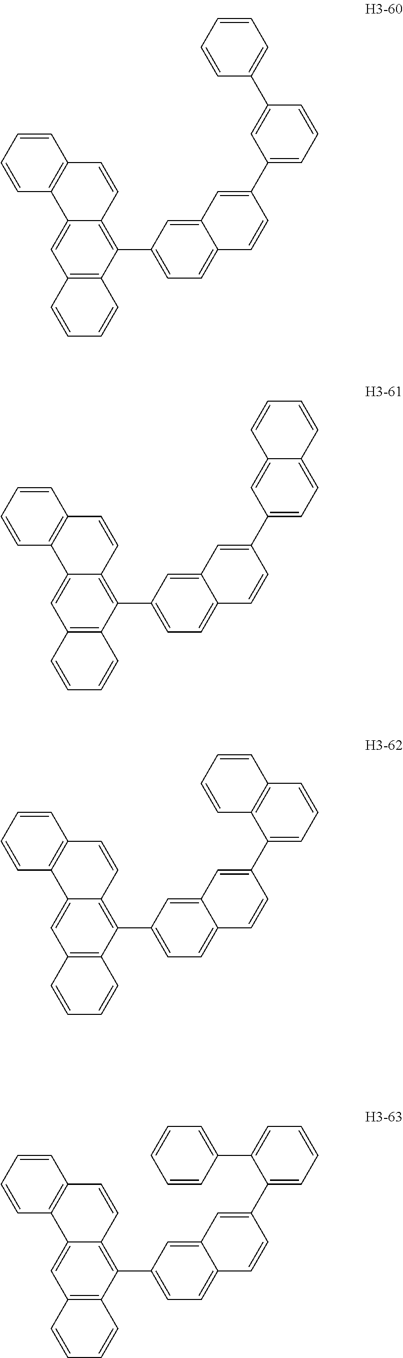 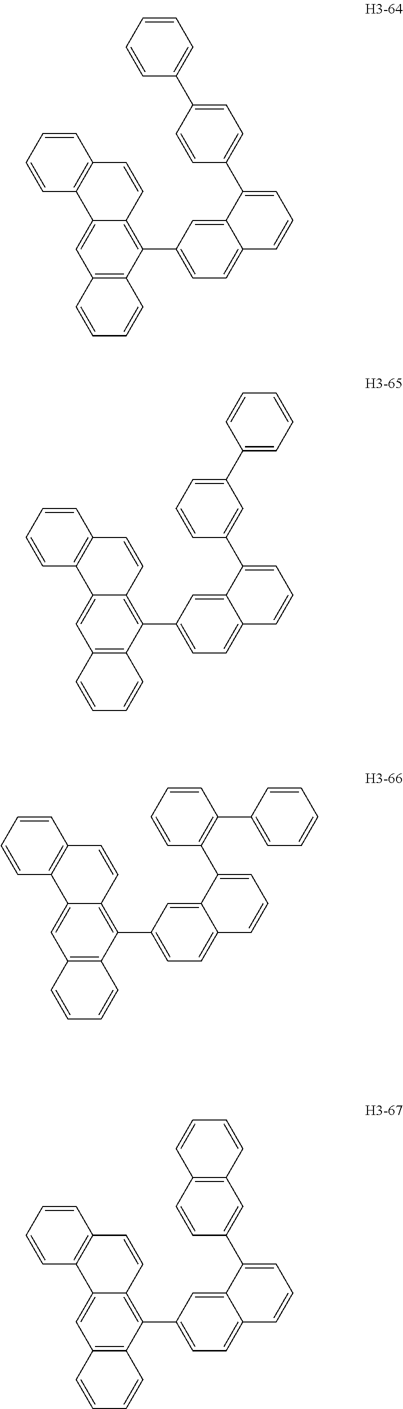 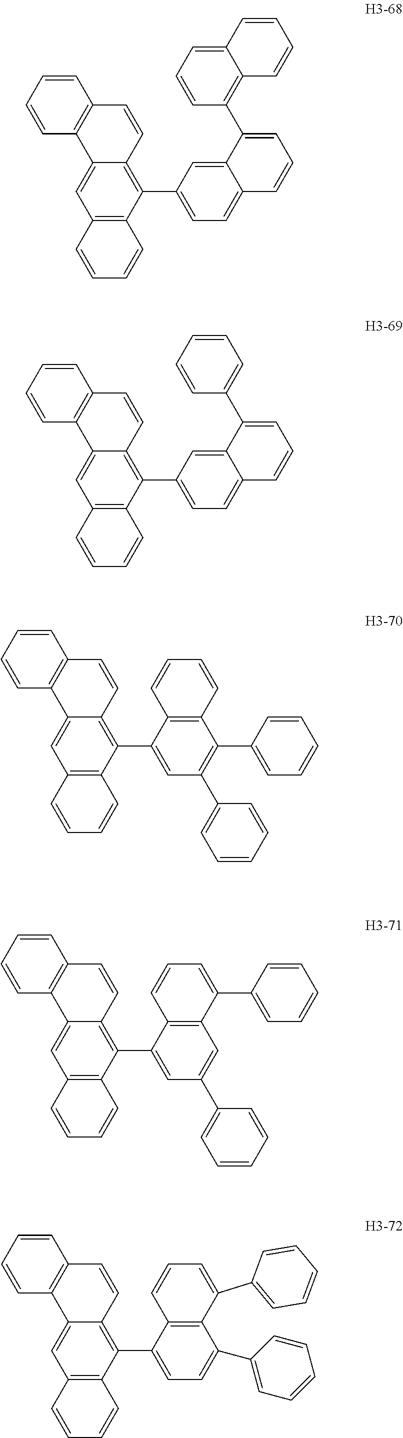 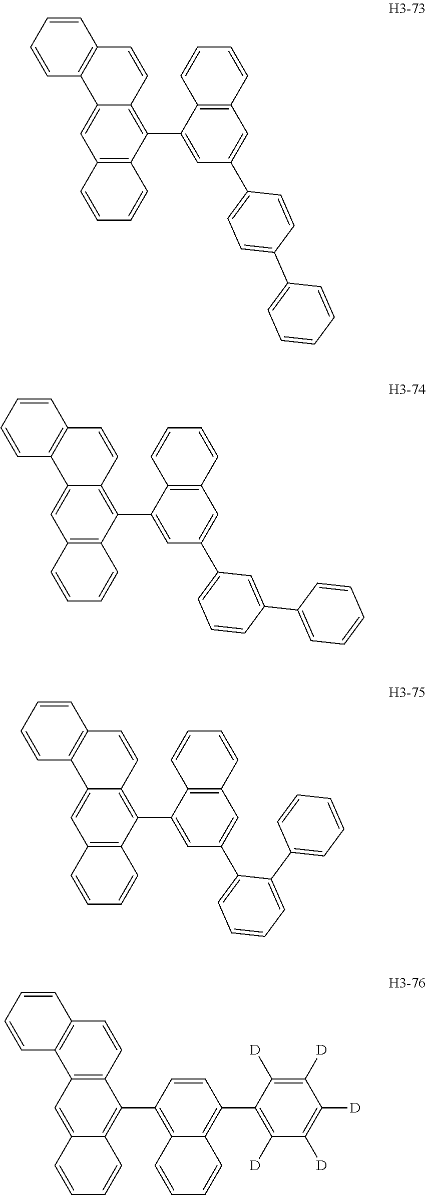 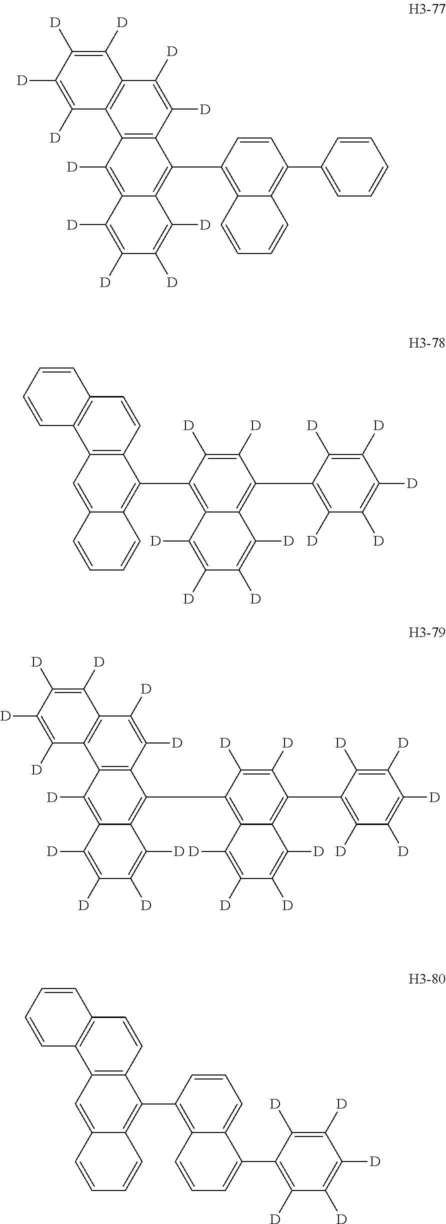 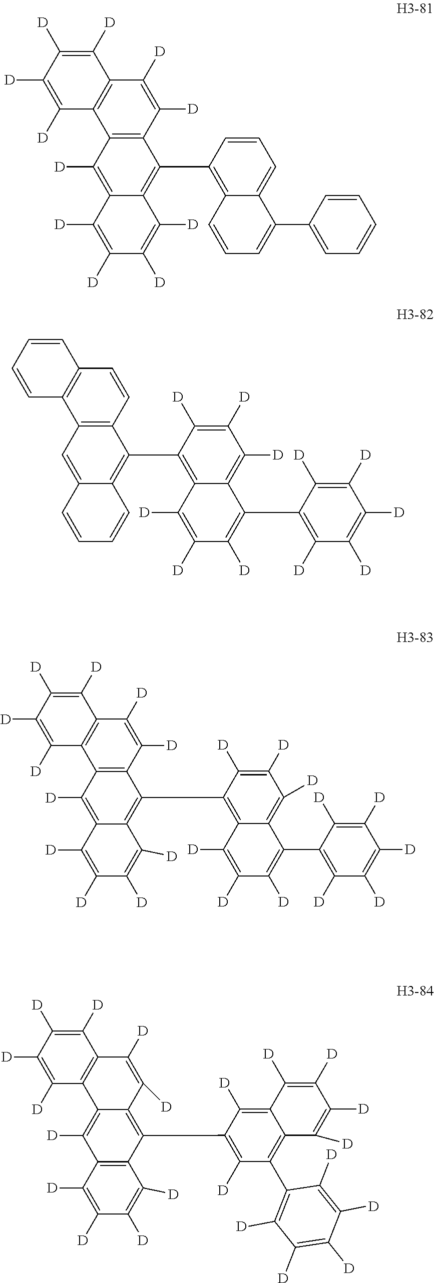 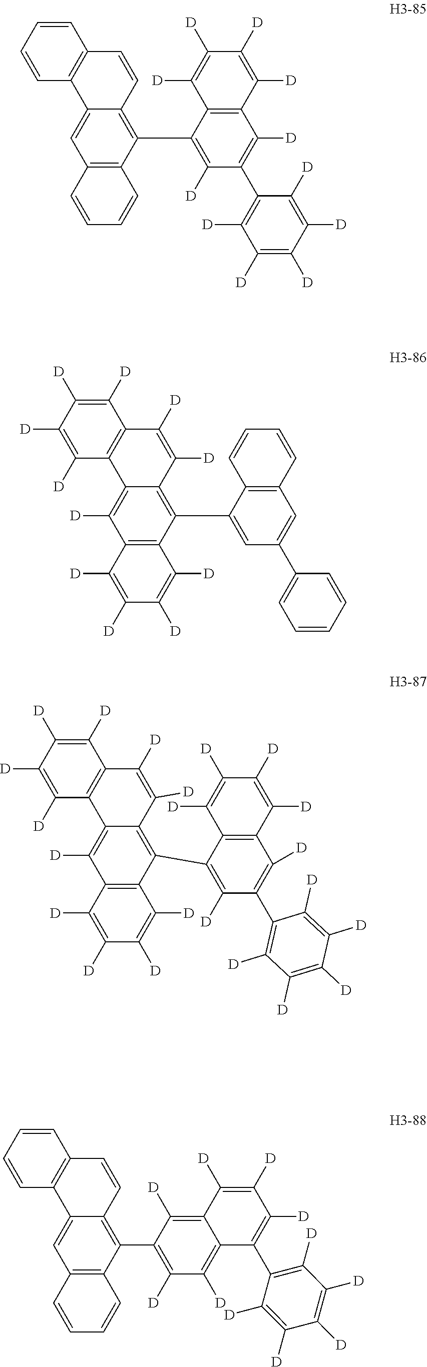 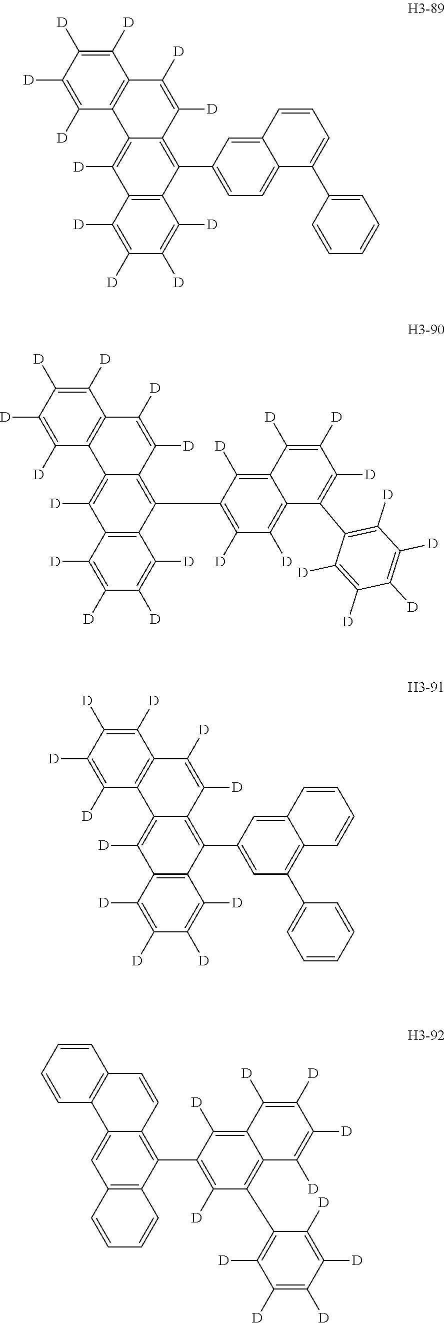 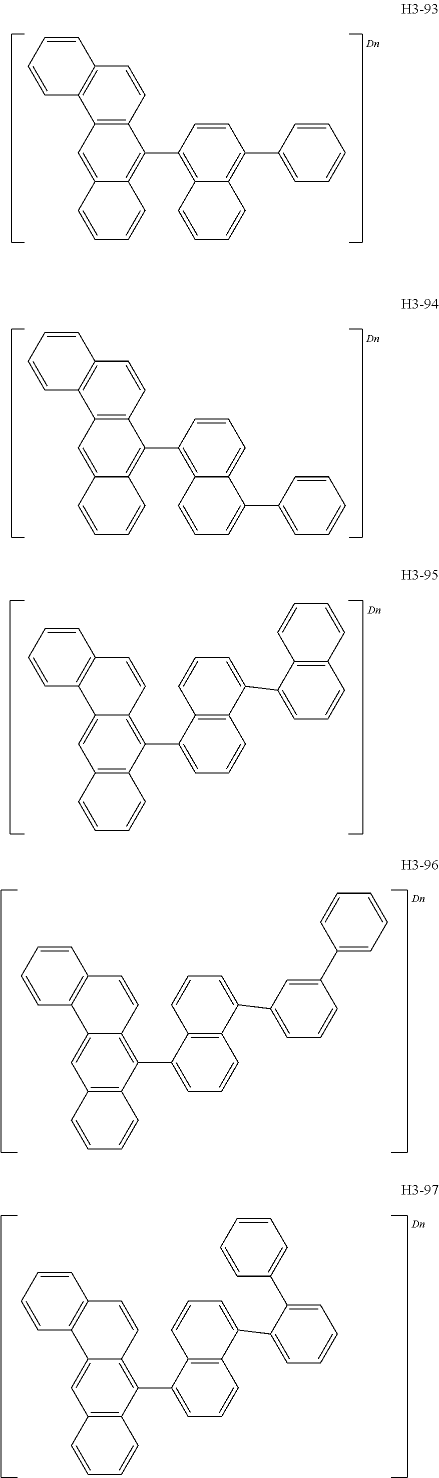 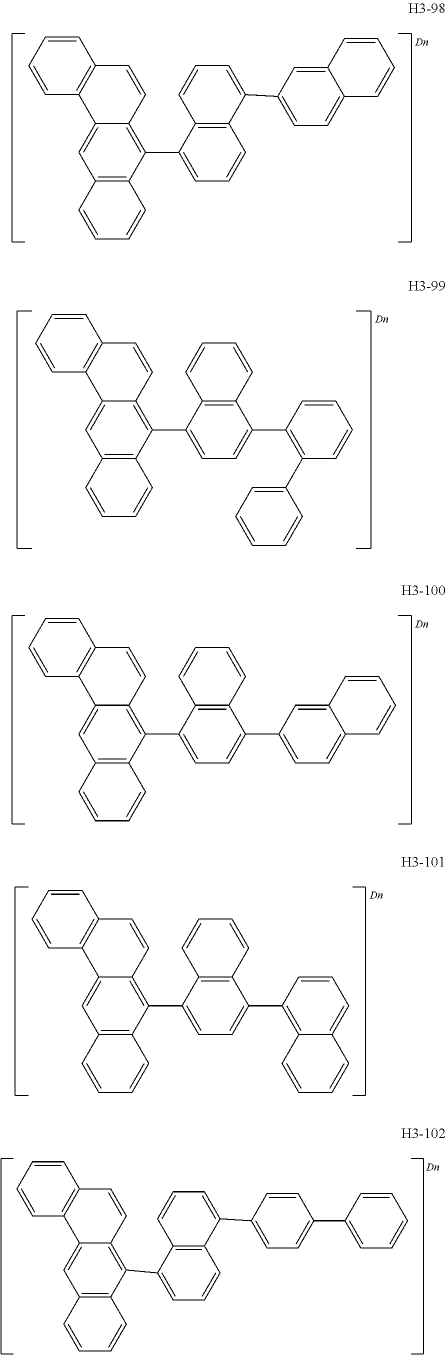 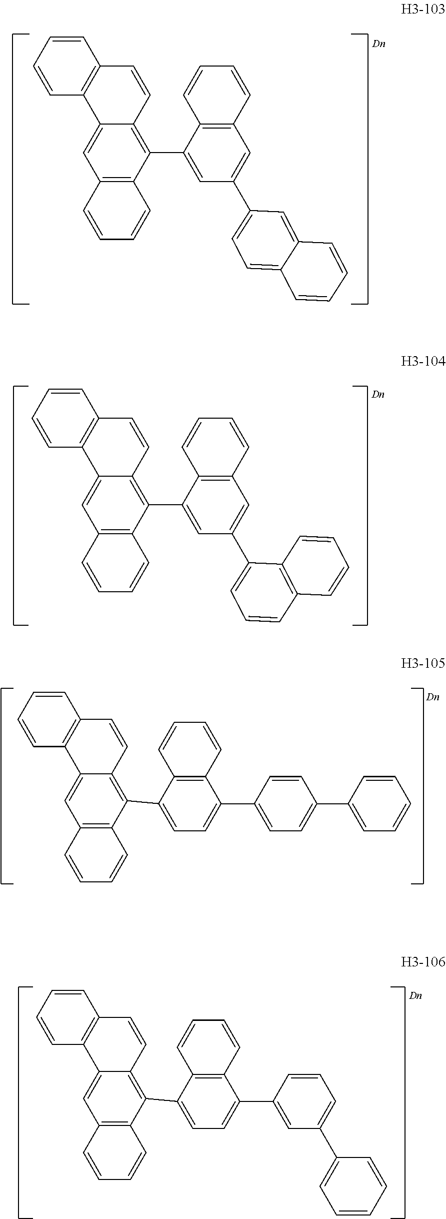 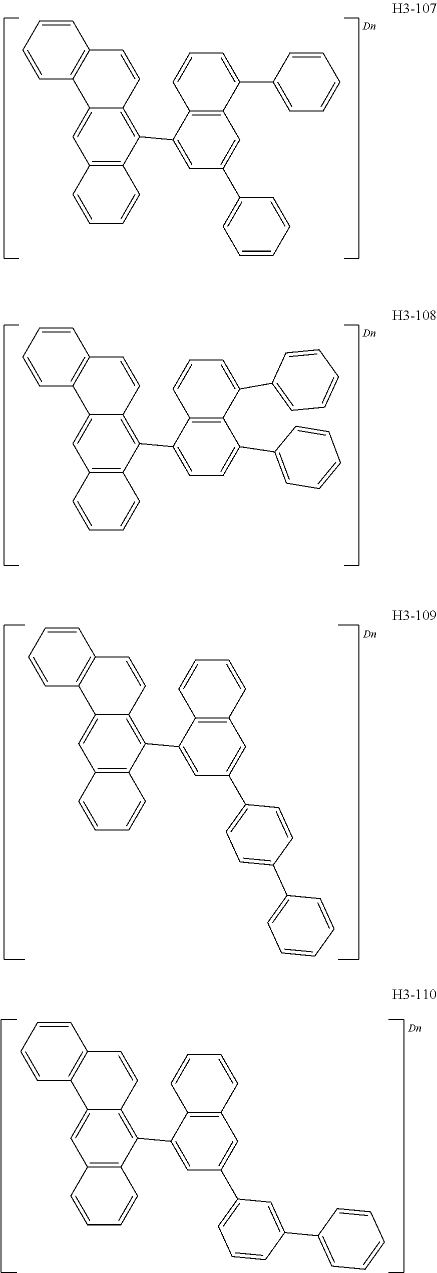 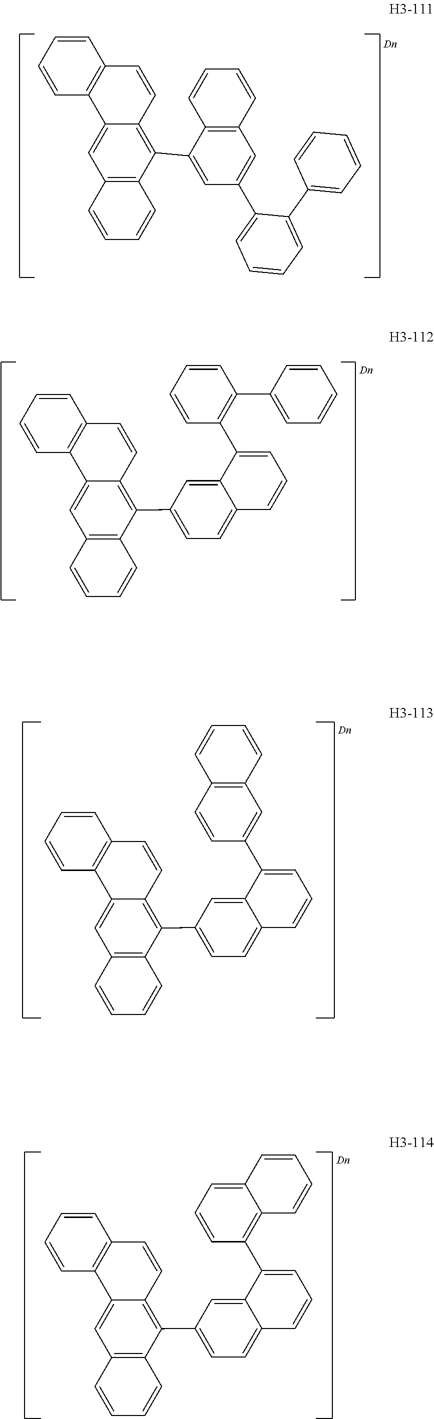 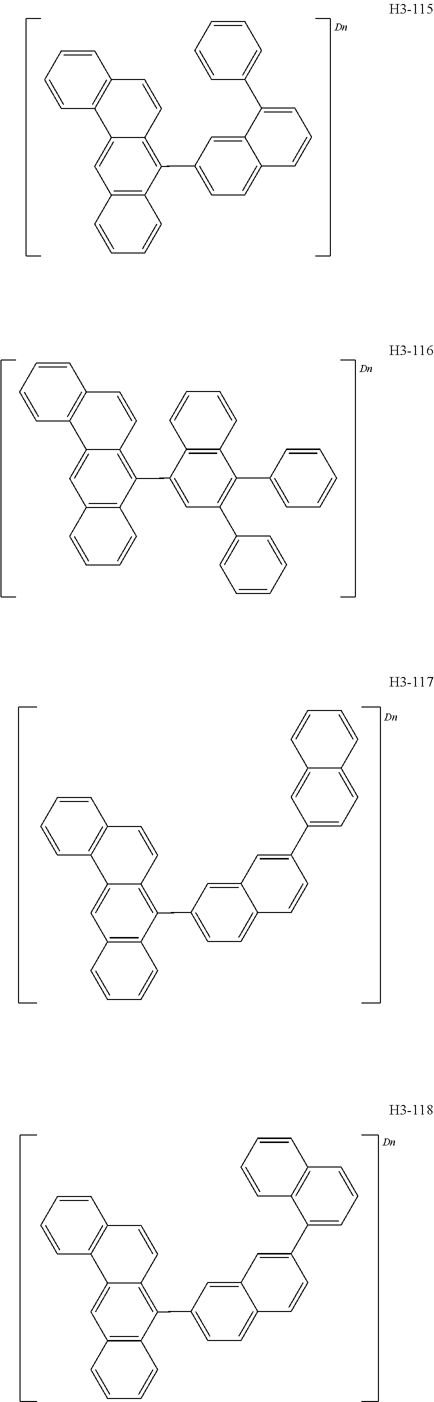 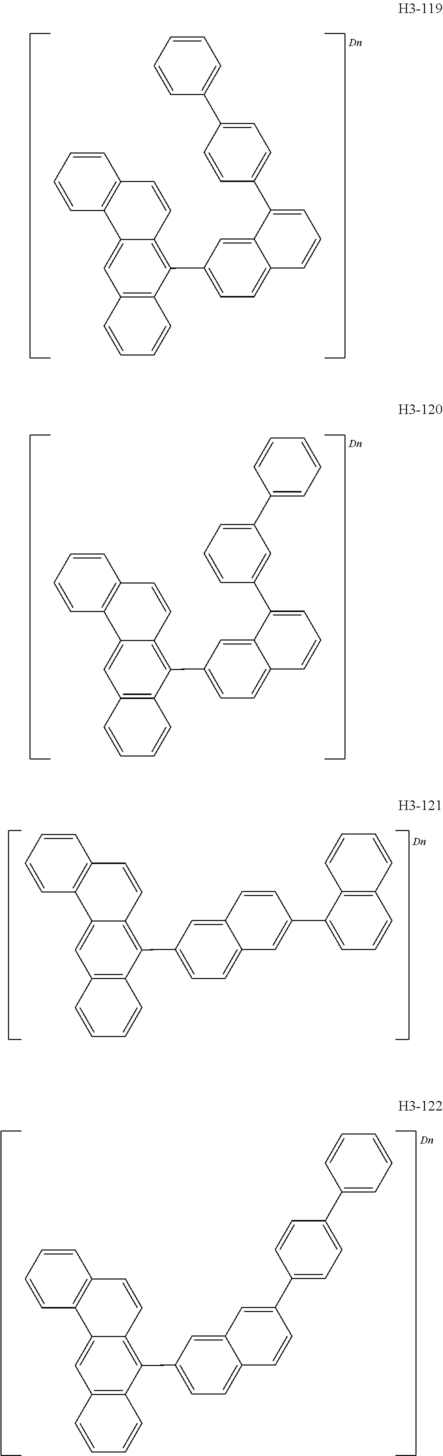 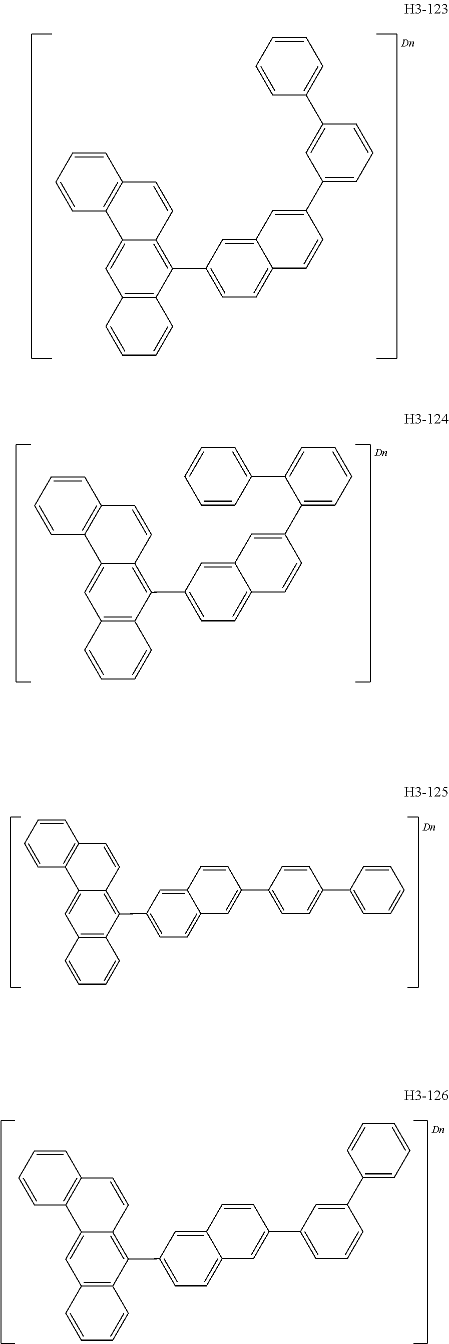 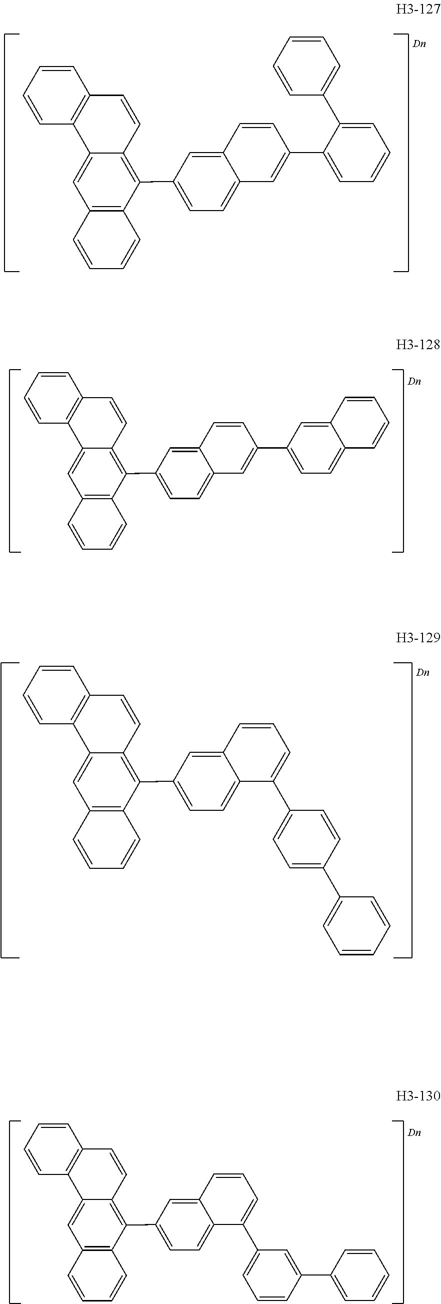 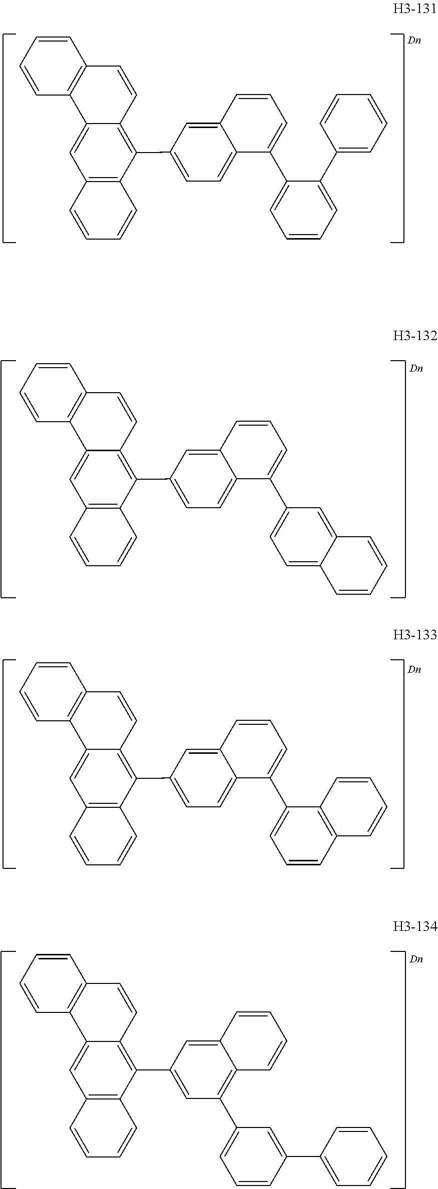 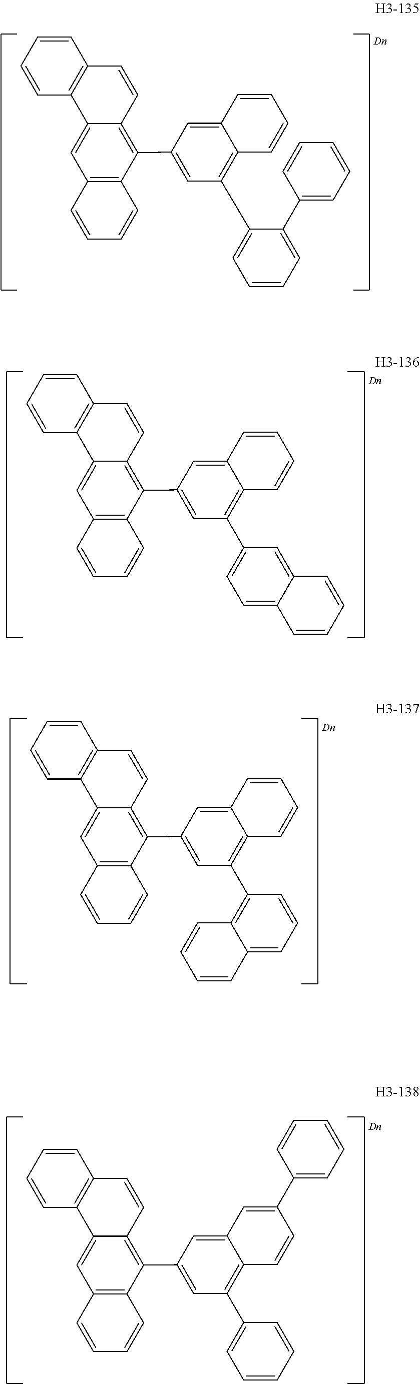 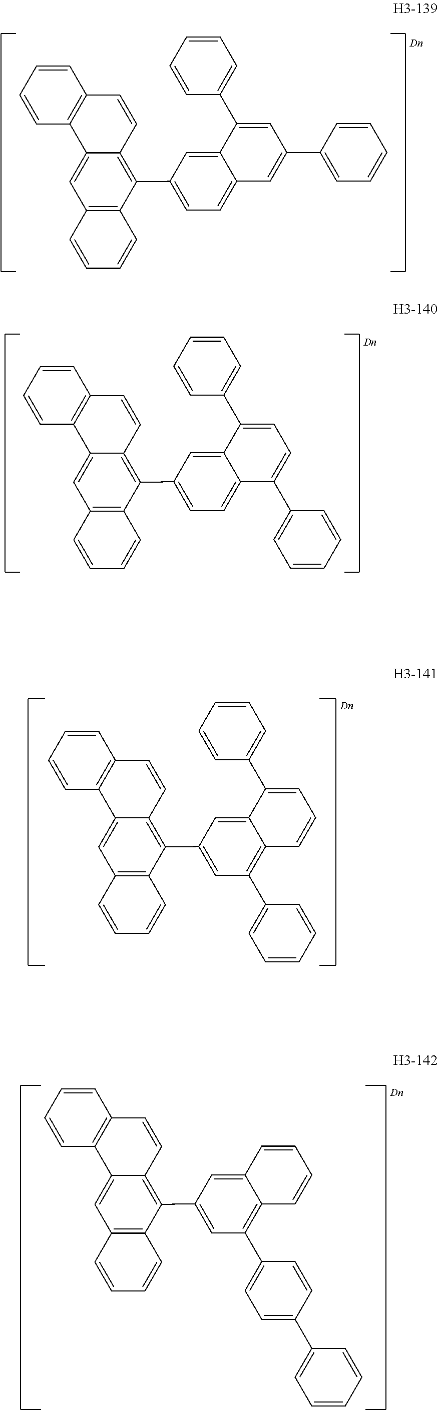
wherein Dₙ means that n hydrogens are substituted with deuterium, and n is an integer from 1 to the maximum number of hydrogens in the compound.

11. An organic electroluminescent device comprising an anode; a cathode; a first light-emitting layer disposed between the anode and the cathode; and a second light-emitting layer disposed between the first light-emitting layer and the cathode, wherein the first light-emitting layer comprises the organic electroluminescent compound according to claim 9, the second light-emitting layer comprises at least two different compounds comprising an anthracene skeleton, and the first light-emitting layer and the second light-emitting layer are in direct contact.

12. An organic electroluminescent compound represented by the following Formula 2-a: 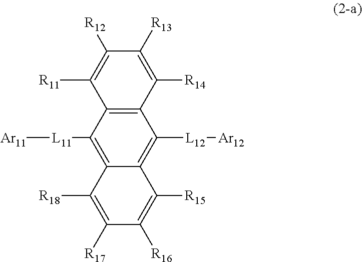
wherein,
Ar₁₁ represents a substituted or unsubstituted (C₆-C₃₀)aryl or a substituted or unsubstituted (3- to 30-membered)heteroaryl;
L₁₁ represents a single bond, a substituted or unsubstituted (C₆-C₃₀)arylene, or a substituted or unsubstituted (3- to 30-membered)heteroarylene;
R₁₁ to R₁₈ each independently represent hydrogen or deuterium;
L₁₂ represents a single bond or a substituted or unsubstituted (C₆-C₃₀)arylene; and
Ar₁₂ is represented by the following Formula 2-b: 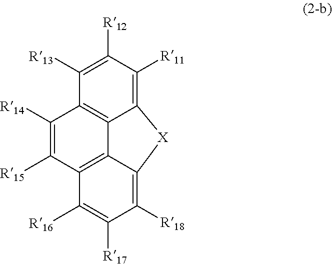
wherein,
X is O or S;
R′₁₁ to R′₁₈ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a combination thereof; or may be linked to the adjacent substituents to form a ring(s);
one of R′₁₁ to R′₁₃ is linked to L₁₂; and
at least one of the remainder of R′₁₁ to R′₁₃ not linked to L₁₂ and R′₁₄ to R′₁₈ is a substituted or unsubstituted (C₁-C₃₀)alkyl, a substituted or unsubstituted (C₃-C₃₀)cycloalkyl, a substituted or unsubstituted (C₆-C₃₀)aryl, a substituted or unsubstituted (C₆-C₃₀)heteroaryl, or a combination thereof.

13. The organic electroluminescent compound according to claim 12, wherein the compound represented by Formula 2-a is selected from the following compounds: 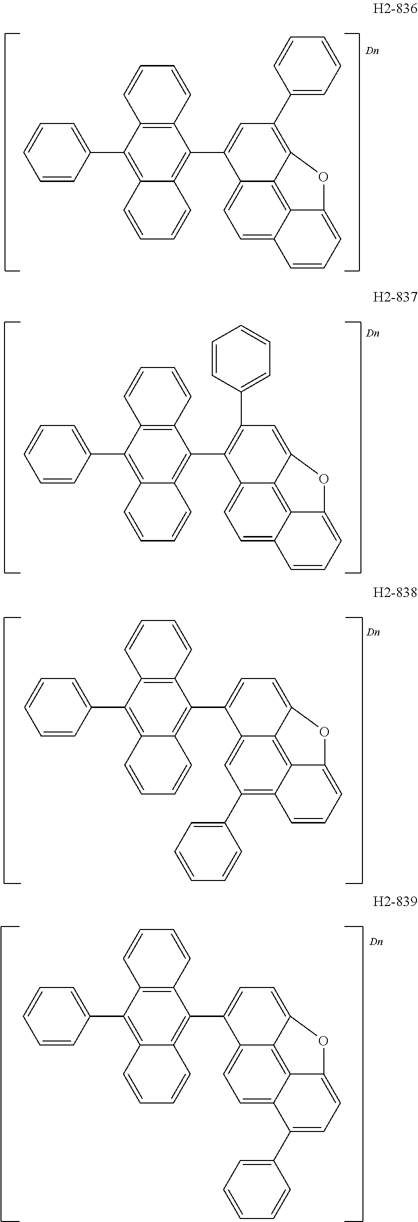 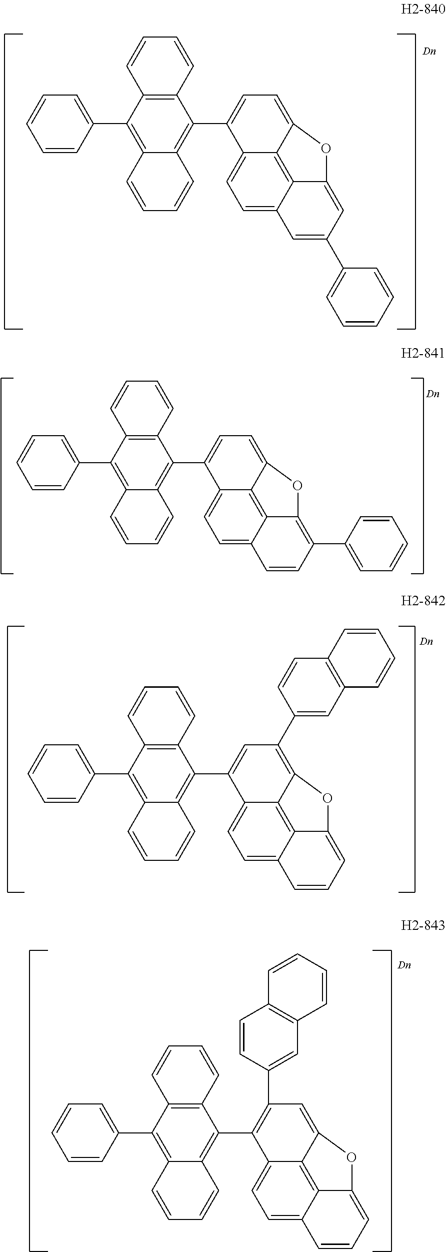 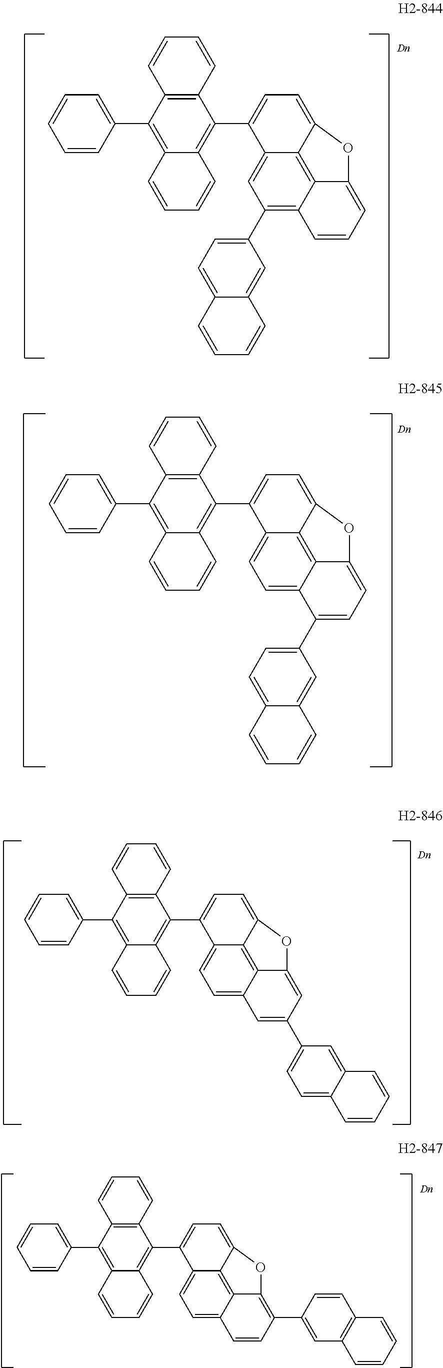 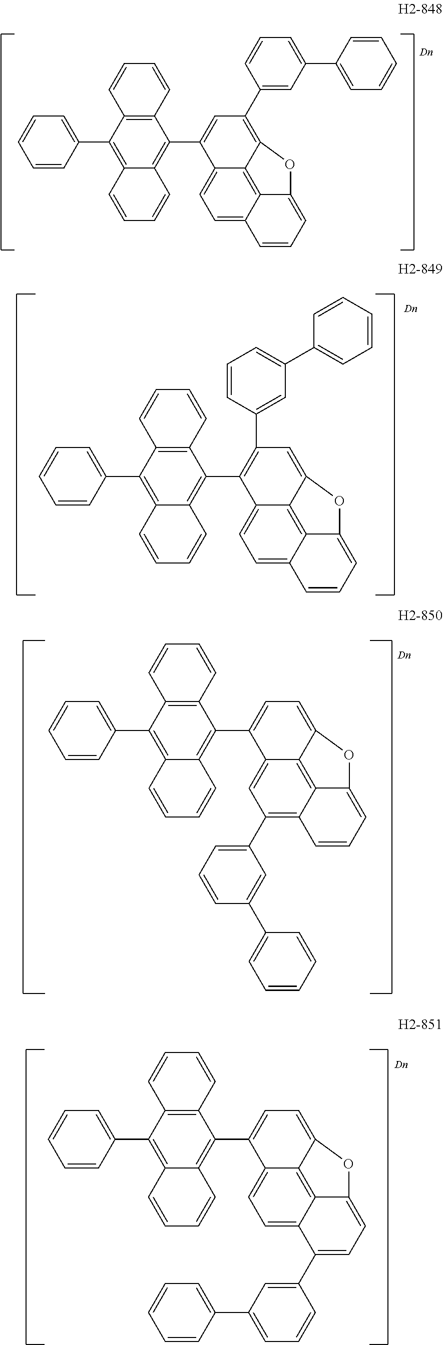 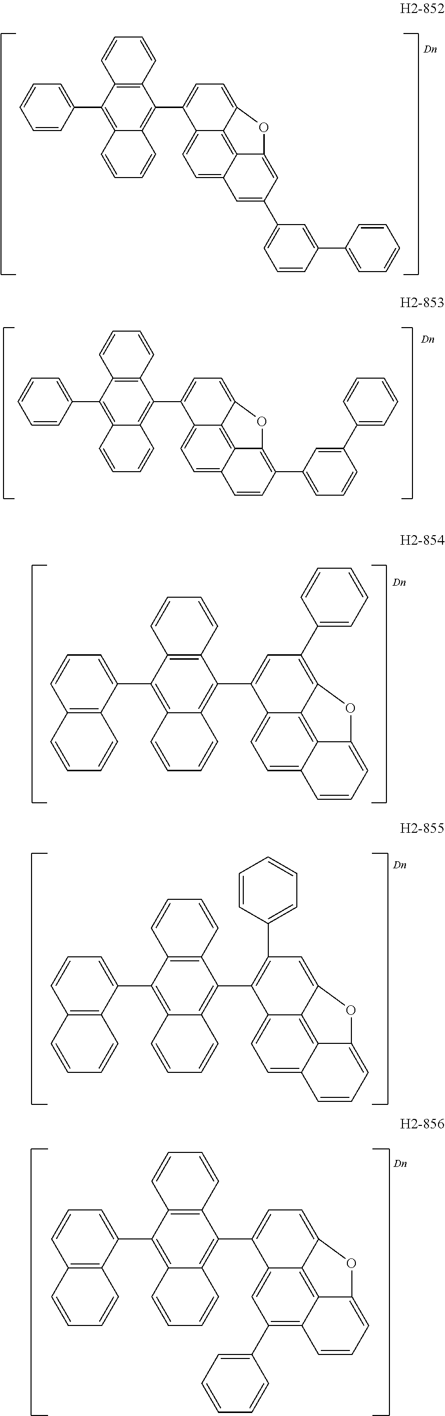 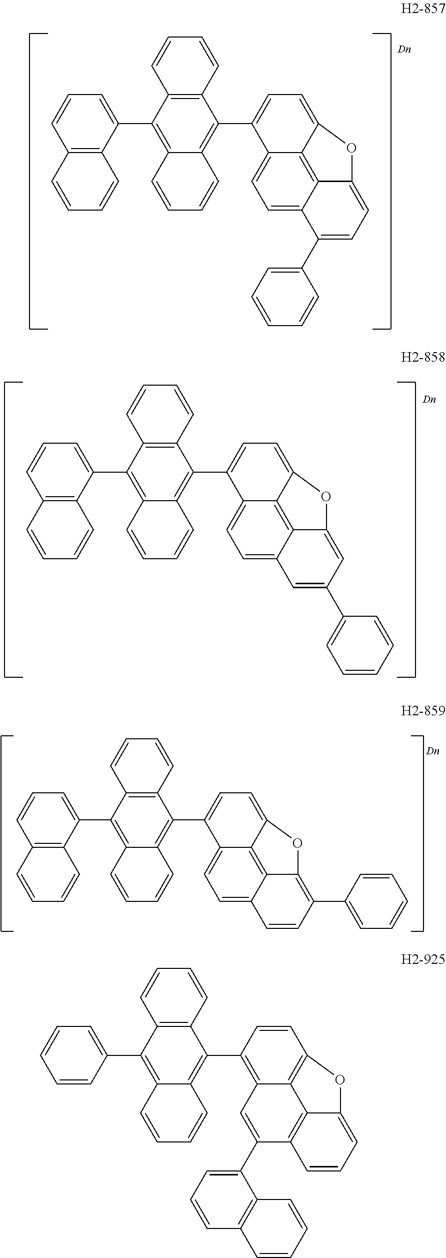 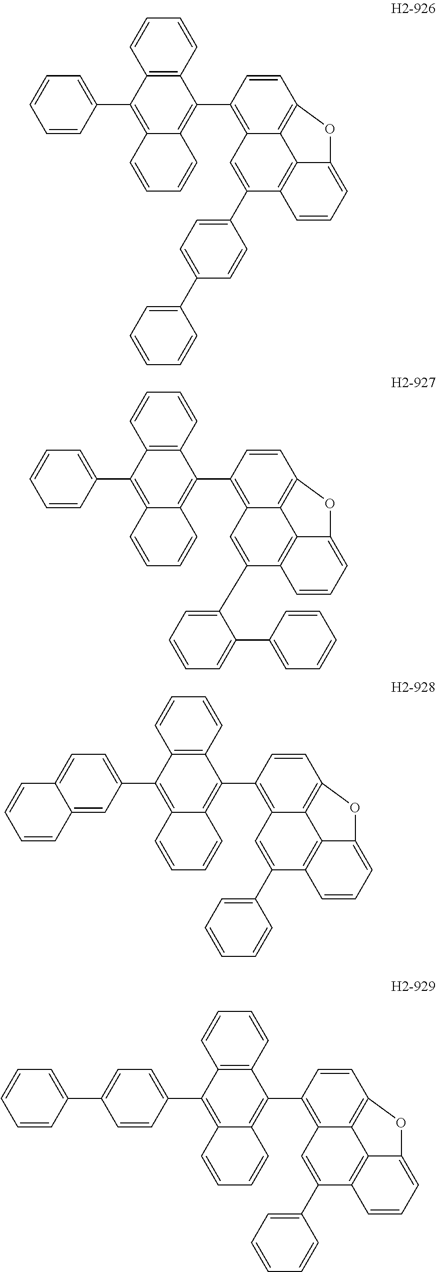 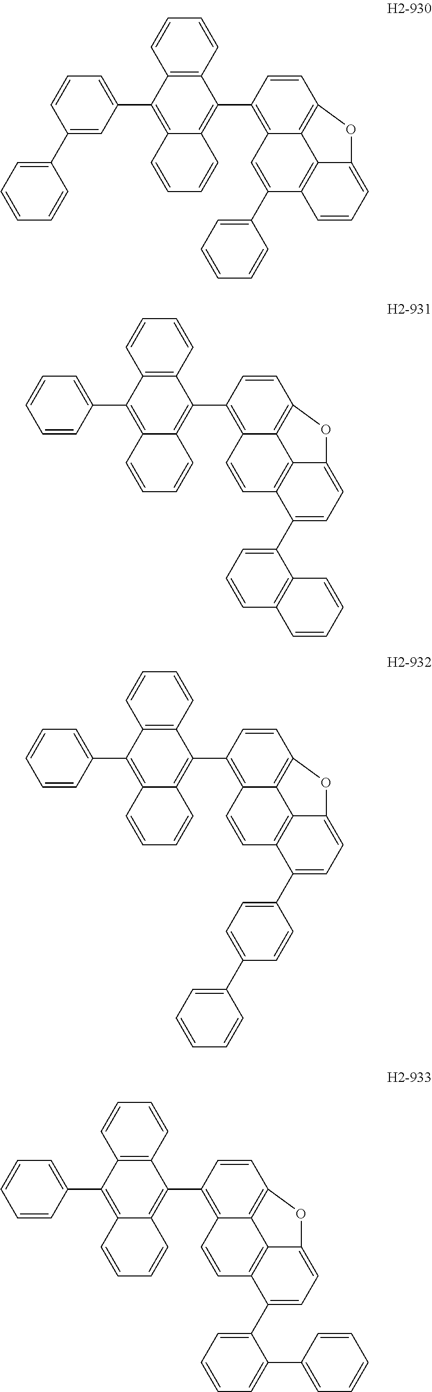 [SMILES: c1ccc(-c2cccc(-c3c4ccccc4c(-c4ccc5oc6cccc7c(-c8ccccc8)cc4c5c67)c4ccccc34)c2)cc1 ; c1ccc(-c2c3ccccc3c(-c3ccc4oc5ccc(-c6cccc7ccccc67)c6ccc3c4c56)c3ccccc23)cc1 ; c1ccc(-c2ccc(-c3ccc4oc5ccc(-c6c7ccccc7c(-c7ccccc7)c7ccccc67)c6ccc3c4c56)cc2)cc1 ; c1ccc(-c2ccccc2-c2ccc3oc4ccc(-c5c6ccccc6c(-c6ccccc6)c6ccccc56)c5ccc2c3c45)cc1] 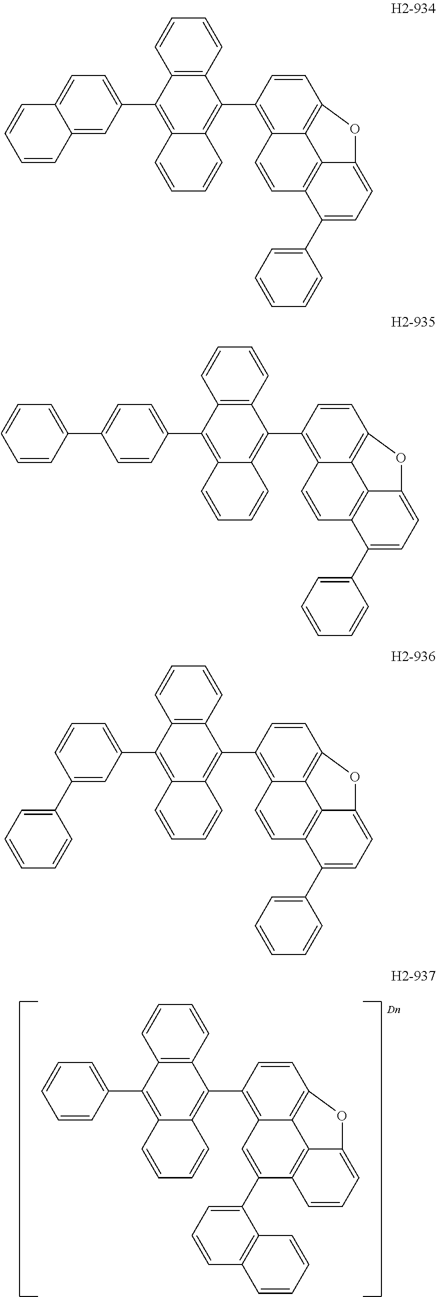 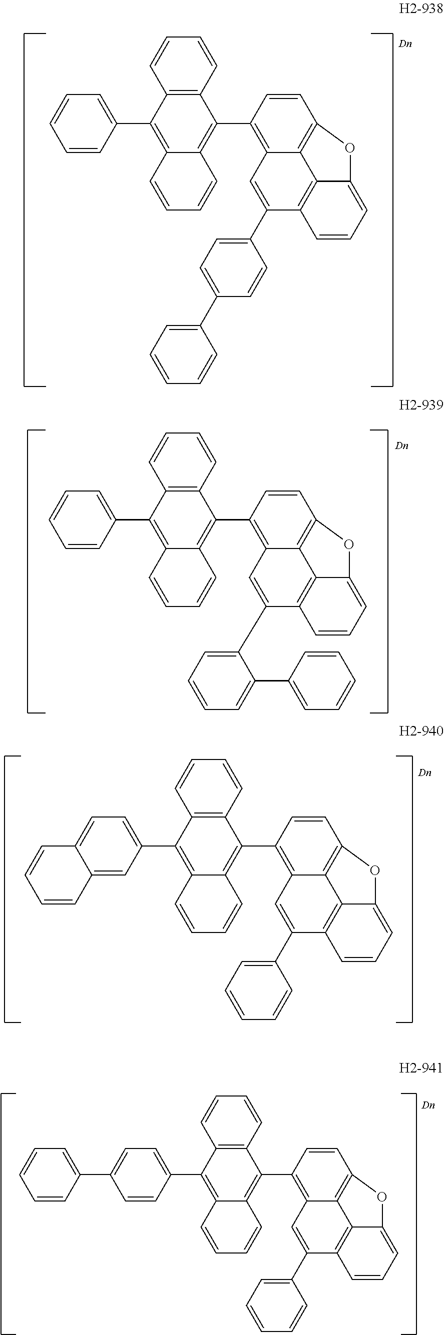 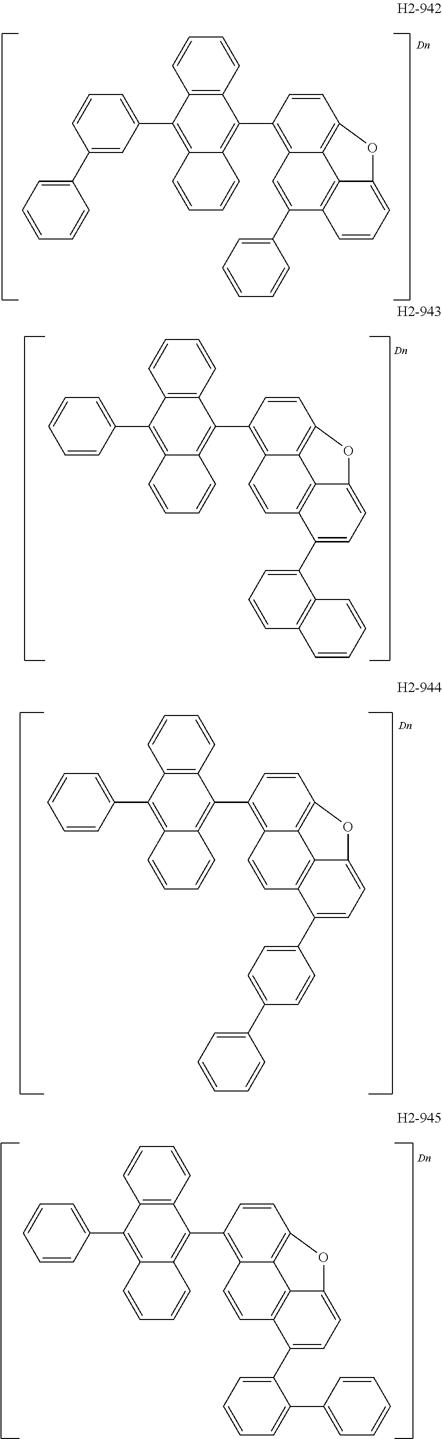 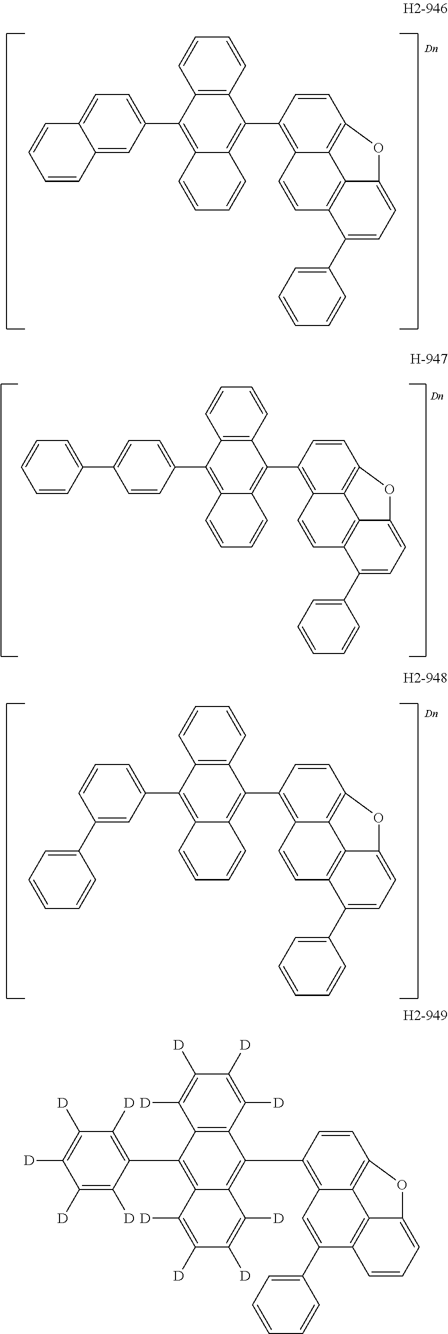 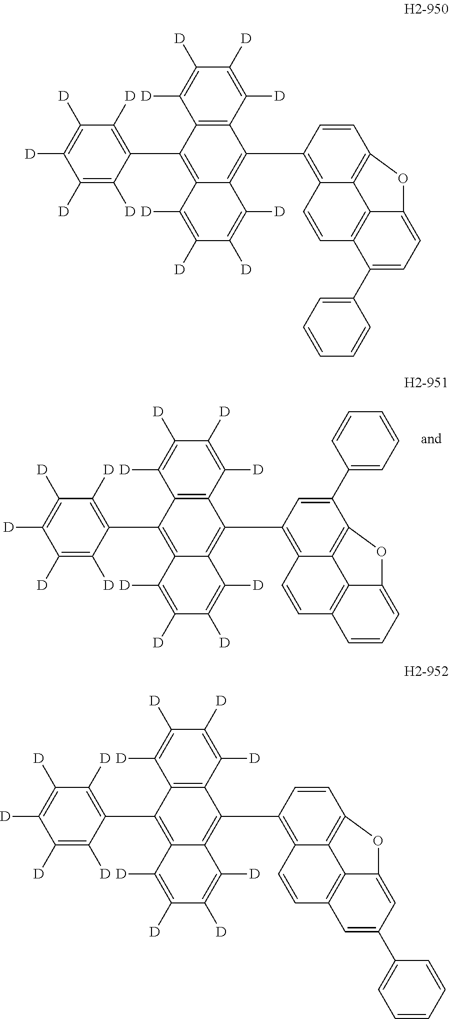 [SMILES: [2H]c1c([2H])c([2H])c(-c2c3c([2H])c([2H])c([2H])c([2H])c3c(-c3ccc4oc5ccc(-c6ccccc6)c6ccc3c4c56)c3c([2H])c([2H])c([2H])c([2H])c23)c([2H])c1[2H] ; [2H]c1c([2H])c([2H])c(-c2c3c([2H])c([2H])c([2H])c([2H])c3c(-c3cc(-c4ccccc4)c4oc5cccc6ccc3c4c65)c3c([2H])c([2H])c([2H])c([2H])c23)c([2H])c1[2H] ; [2H]c1c([2H])c([2H])c(-c2c3c([2H])c([2H])c([2H])c([2H])c3c(-c3ccc4oc5cc(-c6ccccc6)cc6ccc3c4c65)c3c([2H])c([2H])c([2H])c([2H])c23)c([2H])c1[2H]]
wherein in Dₙ, n means the number of substituted deuterium, and n means a number from 0 to the maximum number of deuterium in the compound, where n being 0 means a hydrogen compound.

14. An organic electroluminescent device comprising an anode; a cathode; a first light-emitting layer disposed between the anode and the cathode; and a second light-emitting layer disposed between the first light-emitting layer and the cathode, wherein the second light-emitting layer comprises the organic electroluminescent compound according to claim 12, and the first light-emitting layer and the second light-emitting layer are in direct contact.
